(19) United States
(12) Patent Application Publication
(10) Pub. No.: US 2025/0026814 A1
(43) Pub. Date: Jan. 23, 2025

(54) POLYPEPTIDES FOR DETECTION AND TREATMENT OF CORONAVIRUS INFECTION

(71) Applicant: THE UNIVERSITY OF CHICAGO, Chicago, IL (US)

(72) Inventors: Patrick WILSON, Chicago, IL (US); Siriruk CHANGROB, Chicago, IL (US); Haley DUGAN, Chicago, IL (US); Christopher STAMPER, Chicago, IL (US)

(73) Assignee: THE UNIVERSITY OF CHICAGO, Chicago, IL (US)

(21) Appl. No.: 18/710,900

(22) PCT Filed: Nov. 16, 2022
(86) PCT No.: PCT/US2022/079916

§ 371 Date: May 16, 2024

Related U.S. Application Data
(60) Provisional application No. 63/264,173, filed on Nov. 16, 2021.

(51) Int. Cl. C07K 16/10; A61K 31/573; A61K 31/675; A61K 39/00; A61P 31/14; G01N 33/569

(52) U.S. Cl. CPC C07K 16/1003; A61K 31/573; A61K 31/675; A61P 31/14; G01N 33/56983; A61K 2039/505; C07K 2317/14; C07K 2317/24; C07K 2317/31; C07K 2317/35; C07K 2317/565; C07K 2317/567; C07K 2317/76; C07K 2317/92; G01N 2333/165

(57) ABSTRACT

Here, the inventors report that natural WT SARS-CoV-2 infection induces memory B cells expressing potently neutralizing antibodies against VOCs. Moreover, natural WT infection largely induced antibodies against spike epitopes outside of the RBD, most of which were non-neutralizing against WT and VOCs. Additionally. RBD-binding antibodies could be categorized into 3 distinct classes based on their binding profiles against RBD mutant constructs. The inventors identified VOC-neutralizing antibodies against three distinct regions of the spike protein, including the two epitopes on the RBD and one epitope in the NTD. Together, this study identifies that natural WT infection induces memory B cells that can produce neutralizing antibodies against recent SARS-CoV-2 VOCs and have the potential to be recalled by vaccination.

### CROSS-REFERENCE TO RELATED APPLICATIONS

This application claims the benefit of priority of U.S. Provisional Patent Application No. 63/264,173 filed Nov. 16, 2021, which is hereby incorporated by reference in its entirety.

### STATEMENT OF GOVERNMENT SUPPORT

This invention was made with government support under Grant Numbers 75N93019C00062 and 75N93019C00051, awarded by the National Institutes of Health. The government has certain rights in the invention.

### SEQUENCE LISTING

The application contains a Sequence Listing prepared in compliance with ST.26 format and is hereby incorporated by reference in its entirety. Said Sequence Listing, created on Nov. 11, 2022 is named ARCDP0724WO.xml and is 1,777,457 bytes in size.

### BACKGROUND

### I. Field of the Invention

Aspects of the invention relate to at least the fields of virology and molecular biology.

### II. Background

The emergence of novel circulating SARS-CoV-2 variants of concern (VOCs) have recently proven to undermine the protective effects of infection- and vaccination-induced humoral immunity¹⁻⁴. All approved vaccines against SARS-CoV-2 drive a neutralizing antibody response against the spike protein, the major target of neutralizing antibodies elicited by natural infection^{3, 5}. However, protective humoral immunity against the spike protein induced by vaccination or infection with the original wildtype (WT) virus may be attenuated due to the widespread circulation of variants². The first reported mutation of the SARS-CoV-2 spike protein, D614G, arose in the C-terminal domain (CTD) and evolved due to increased stability of the spike rather than a mutation to escape host immunity⁶. More recently, mutations have arisen within the receptor-binding domain (RBD), N-terminal domain (NTD) of S1, and S2 that have resulted in emergence of several circulating viral variants that are rapidly becoming the dominant strains around the globe². The B.1.1.7 lineage or alpha VOC, first found in the United Kingdom, has been reported to have a >50% increased transmissibility among humans⁷⁻¹⁰. Of greatest concern is the substitution at position 484 in the RBD, which is exclusively shared by the VOCs, variants of interest (VOIs) and variants under monitoring (VUMs) originally identified in South Africa (B.1.351; beta), Brazil (P.1; gamma), Texas (R.1), Columbia (B.1.621; mu), New York (B.1.526; iota) and India (B.1.617.1; kappa)^{2, 3, 11-15}. VOCs possessing a mutation at E484, either E484K and E484Q, can partially evade neutralizing humoral immunity induced by either natural infection or vaccination and, in rare cases, lead to reinfection and infection, respectively^{11-13, 16-18}. Other emerging variants have acquired a mutation at L452R within the RBD, which is found in B.1.1.298, a variant capable of interspecies transmission between humans and minks, and B.1.427/B.1.429 (epsilon) isolated in southern California¹⁹. Moreover, the B.1.617.1 (kappa) found in India possesses both L452R and E484Q mutations within the RBD^{15, 20}. The most recent VOC, B.1.617.2 (delta), is responsible for a surge in both cases and fatalities in several countries, especially where vaccination rates are low^{4, 21-23}. Intriguingly, the B. 1.617 lineages contain P681R, a mutation that enhances and accelerates viral fusion²⁴ and which is also present in the dominant variant in Uganda, A.23.1²⁵. Thus, understanding the impact of these various mutations on the neutralization capacity of antibodies elicited by current vaccine formulations or natural exposure to wildtype (WT) SARS-CoV-2 is urgently needed to develop critical next-generation vaccine strategies against SARS-CoV-2 variants.

### SUMMARY

Here, the inventors report that natural WT SARS-CoV-2 infection induces memory B cells expressing potently neutralizing antibodies against VOCs. Moreover, natural WT infection largely induced antibodies against spike epitopes outside of the RBD, most of which were non-neutralizing against WT and VOCs. Additionally, RBD-binding antibodies could be categorized into 3 distinct classes based on their binding profiles against RBD mutant constructs. The inventors identified VOC-neutralizing antibodies against three distinct regions of the spike protein, including the two epitopes on the RBD and one epitope in the NTD. Together, this study identifies that natural WT infection induces memory B cells that can produce neutralizing antibodies against recent SARS-CoV-2 VOCs and have the potential to be recalled by vaccination.

The disclosure describes novel antibody and antigen binding fragments. Also described are polypeptides comprising the antigen binding fragment(s) of the disclosure, and compositions comprising the polypeptides, antibodies, and/or antigen binding fragments of the disclosure. Also described are nucleic acids encoding an antibody or antigen binding fragment of the disclosure. The disclosure also relates to nucleic acids encoding an antibody heavy chain, wherein the nucleic acid has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of the nucleic acid sequences of a heavy chain of Table 2. Also described are nucleic acids encoding an antibody light chain of the disclosure, wherein the nucleic acid has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of the nucleic acid sequences of a light chain of Table 2. Also provided are vectors or expression vectors comprising nucleic acids of the disclosure and host cells comprising polypeptides, nucleic acids, vectors, antibodies, or antigen binding fragments of the disclosure. The nucleic acids of the disclosure may be DNA or RNA.

Also described is a method of a making a cell comprising transferring one or more nucleic acid(s) of the disclosure into a cell. The method may further comprise culturing the cell under conditions that allow for expression of a polypeptide from the nucleic acid. The method may further comprise isolating the expressed polypeptide. Also described is a method for producing a polypeptide comprising transferring one or more nucleic acid(s) or vector(s) of the disclosure into a cell and isolating polypeptides expressed from the nucleic acid. Methods also include a method for producing a polypeptide comprising culturing cells comprising nucleic acid(s) or vectors of the disclosure and isolating polypeptides expressed from the nucleic acid. The cell may be further defined as a human cell, B cell, T cell, Chinese hamster ovary, NS0 murine myeloma cell, PER.C6 cell, or a cell described herein.

Methods include a method for treating, preventing, vaccinating against, and/or inducing an immune response against a coronavirus infection in a subject, the method comprising administering to the subject an antibody, antigen binding fragment, polypeptide, nucleic acid, or host cell of the disclosure. Also provided is a method for evaluating a sample from a subject, the method comprising contacting a biological sample from the subject, or extract thereof, with at least one antibody, antigen binding fragment, or polypeptide of the disclosure. Also disclosed is a method for diagnosing a SARS-CoV-2 infection in a subject, the method comprising contacting a biological sample from the subject, or extract thereof, with at least one antibody, antigen binding fragment, or polypeptide of any one of the disclosure. The compositions of the disclosure may be formulated as a vaccine for the treatment or prevention of a coronavirus infection. The antibodies, antigen binding fragments, or compositions of the disclosure may be used in a vaccine for preventing coronaviral infections in a subject that does not have a coronaviral infection. The antibodies, antigen binding fragments, or compositions of the disclosure may be used to treat a subject having a coronaviral infection.

The disclosure describes an antibody or antigen binding fragment comprising a heavy chain variable region and a light chain variable region: (i) wherein the heavy chain variable region comprises a HCDR1, HCDR2, and HCDR3 having at least 80% sequence identity to the HCDR1, HCR2, HCR3 of SEQ ID NOs: 1565, 1566, and 1567 and wherein the light chain variable region comprises a LCDR1, LCDR2, and LCDR3 having at least 80% sequence identity to the LCDR1, LCDR2, and LCDR3 of SEQ ID NOs: 1572, 1573, and 1574; (ii) wherein the heavy chain variable region comprises a HCDR1, HCDR2, and HCDR3 having at least 80% sequence identity to the HCDR1, HCR2, HCR3 of SEQ ID NOs: 1457, 1458, and 1459 and wherein the light chain variable region comprises a LCDR1, LCDR2, and LCDR3 having at least 80% sequence identity to the LCDR1, LCDR2, and LCDR3 of SEQ ID NOs: 1464, 1465, and 1466; or (iii) wherein the heavy chain variable region comprises a HCDR1, HCDR2, and HCDR3 having at least 80% sequence identity to the HCDR1, HCR2, HCR3 of SEQ ID NOs: 1492, 243, and 1493 and wherein the light chain variable region comprises a LCDR1, LCDR2, and LCDR3 having at least 80% sequence identity to the LCDR1, LCDR2, and LCDR3 of SEQ ID NOs: 1497, 1498, and 1499.

The disclosure describes an antibody or antigen binding fragment comprising a heavy chain variable region and a light chain variable region: (i) wherein the heavy chain variable region comprises a HCDR1, HCDR2, and HCDR3 having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the HCDR1, HCR2, HCR3 of SEQ ID NOs: 1565, 1566, and 1567 and wherein the light chain variable region comprises a LCDR1, LCDR2, and LCDR3 having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LCDR1, LCDR2, and LCDR3 of SEQ ID NOs: 1572, 1573, and 1574; (ii) wherein the heavy chain variable region comprises a HCDR1, HCDR2, and HCDR3 having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the HCDR1, HCR2, HCR3 of SEQ ID NOs: 1457, 1458, and 1459 and wherein the light chain variable region comprises a LCDR1, LCDR2, and LCDR3 having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LCDR1, LCDR2, and LCDR3 of SEQ ID NOs: 1464, 1465, and 1466; or (iii) wherein the heavy chain variable region comprises a HCDR1, HCDR2, and HCDR3 having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the HCDR1, HCR2, HCR3 of SEQ ID NOs: 1492, 243, and 1493 and wherein the light chain variable region comprises a LCDR1, LCDR2, and LCDR3 having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LCDR1, LCDR2, and LCDR3 of SEQ ID NOs: 1497, 1498, and 1499.

The antibody or antigen binding fragment may have a heavy chain variable region comprising a HCDR1, HCDR2, and HCDR3 having the amino acid sequence of SEQ ID NOs: 1565, 1566, and 1567 and a light chain variable region comprising a LCDR1, LCDR2, and LCDR3 having the amino acid sequence of SEQ ID NOs: 1572, 1573, and 1574. The antibody or antigen binding fragment may have a heavy chain variable region comprising a HCDR1, HCDR2, and HCDR3 having the amino acid sequence of SEQ ID NOs: 1457, 1458, and 1459 and a light chain variable region comprising a LCDR1, LCDR2, and LCDR3 comprising the amino acid sequence of SEQ ID NOs: 1464, 1465, and 1466. The antibody or antigen binding fragment may have a heavy chain variable region comprising a HCDR1, HCDR2, and HCDR3 having the amino acid sequence of SEQ ID NOs: 1492, 243, and 1493 and a light chain variable region having the amino acid sequence of SEQ ID NOs: 1497, 1498, and 1499.

The heavy chain may comprise an amino acid sequence with at least 80% sequence identity to SEQ ID NO: 1563 or 1564 and/or the light chain may comprise an amino acid sequence with at least 80% sequence identity to SEQ ID NO: 1570 or 1571. The heavy chain may comprise an amino acid sequence with at least 80% sequence identity to SEQ ID NO: 1455 or 1456 and/or the light chain may comprise an amino acid sequence with at least 80% sequence identity to SEQ ID NO: 1462 or 1463. The heavy chain may comprise an amino acid sequence with at least 80% sequence identity to SEQ ID NO: 1490 or 1491 and/or the light chain may comprise an amino acid sequence with at least 80% sequence identity to SEQ ID NO:1495 or 1496. The heavy chain may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 1563 or 1564 and/or the light chain may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:1570 or 1571. The heavy chain may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:1455 or 1456 and/or the light chain may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:1462 or 1463. The heavy chain may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 1490 or 1491 and/or the light chain may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:1495 or 1496.

The antibody or antigen binding fragment may comprise a heavy chain comprising the amino acid sequence of SEQ ID NO: 1563 or 1564 and/or a light chain comprising the amino acid sequence of SEQ ID NO:1570 or 1571. The antibody or antigen binding fragment may comprise a heavy chain comprising the amino acid sequence of SEQ ID NO: 1455 or 1456 and/or a light chain comprising the amino acid sequence of SEQ ID NO: 1462 or 1463. The antibody or antigen binding fragment may comprise a heavy chain comprising the amino acid sequence of SEQ ID NO: 1490 or 1491 and/or a light chain comprising the amino acid sequence of SEQ ID NO:1495 or 1496.

The antibody or antigen binding fragment may comprise a heavy chain framework region (HFR) 1, HFR2, HFR3, and HFR4 and light chain framework region (LFR) 1, LFR2, LFR3, and LFR4 and wherein the HFR1, HFR2, HFR3, and HFR4 comprises an amino acid sequence with at least 80% sequence identity to SEQ ID NOs: 1568, 130, 1569, and 60, respectively, and the LFR1, LFR2, LFR3, and LFR4 comprises an amino acid sequence with at least 80% sequence identity to SEQ ID NOs: 1575, 950, 1576, and 69. The antibody or antigen binding fragment may comprise a heavy chain framework region HFR1, HFR2, HFR3, and HFR4 and light chain framework region LFR1, LFR2, LFR3, and LFR4, and wherein the HFR1, HFR2, HFR3, and HFR4 comprises an amino acid sequence with at least 80% sequence identity to SEQ ID NOs: 1460, 1461, 146, and 60, respectively, and the LFR1, LFR2, LFR3, and LFR4 comprises an amino acid sequence with at least 80% sequence identity to SEQ ID NOs: 1467, 1468, 1469, and 53. The antibody or antigen binding fragment may comprise a heavy chain framework region HFR1, HFR2, HFR3, and HFR4 and light chain framework region LFR1, LFR2, LFR3, and LFR4, and wherein the HFR1, HFR2, HFR3, and HFR4 comprises an amino acid sequence with at least 80% sequence identity to SEQ ID NOs: 245, 7, 1494, and 44, respectively, and the LFR1, LFR2, LFR3, and LFR4 comprises an amino acid sequence with at least 80% sequence identity to SEQ ID NOs: 1500, 1501, 1502, and 18.

The antibody or antigen binding fragment may comprise a heavy chain framework region (HFR) 1, HFR2, HFR3, and HFR4 and light chain framework region (LFR) 1, LFR2, LFR3, and LFR4 and wherein the HFR1, HFR2, HFR3, and HFR4 comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs: 1568, 130, 1569, and 60, respectively, and the LFR1, LFR2, LFR3, and LFR4 comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs: 1575, 950, 1576, and 69. The antibody or antigen binding fragment may comprise a heavy chain framework region HFR1, HFR2, HFR3, and HFR4 and light chain framework region LFR1, LFR2, LFR3, and LFR4, and wherein the HFR1, HFR2, HFR3, and HFR4 comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs: 1460, 1461, 146, and 60, respectively, and the LFR1, LFR2, LFR3, and LFR4 comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs: 1467, 1468, 1469, and 53. The antibody or antigen binding fragment may comprise a heavy chain framework region HFR1, HFR2, HFR3, and HFR4 and light chain framework region LFR1, LFR2, LFR3, and LFR4, and wherein the HFR1, HFR2, HFR3, and HFR4 comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs: 245, 7, 1494, and 44, respectively, and the LFR1, LFR2, LFR3, and LFR4 comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs: 1500, 1501, 1502, and 18.

The antibody or antigen binding fragment may comprise a HFR1, HFR2, HFR3, and HFR4 comprising the amino acid sequence of SEQ ID NOs: 1568, 130, 1569, and 60, and a LFR1, LFR2, LFR3, and LFR4 comprising the amino acid sequence of SEQ ID NOs: 1575, 950, 1576, and 69. The antibody or antigen binding fragment may comprise a HFR1, HFR2, HFR3, and HFR4 comprising the amino acid sequence of SEQ ID NOs: 1460, 1461, 146, and 60, and a LFR1, LFR2, LFR3, and LFR4 comprising the amino acid sequence of SEQ ID NOs: 1467, 1468, 1469, and 53. The antibody or antigen binding fragment may comprise a HFR1, HFR2, HFR3, and HFR4 comprising the amino acid sequence of SEQ ID NOs: 245, 7, 1494, and 44, and a LFR1, LFR2, LFR3, and LFR4 comprising the amino acid sequence of SEQ ID NOs: 1500, 1501, 1502, and 18.

The disclosure describes an antibody or antigen binding fragment comprising a heavy chain variable region and a light chain variable region, wherein the heavy chain variable region comprises a HCDR1, HCDR2, and HCDR3 from a heavy chain variable region of an antibody clone of Table 1 and wherein the light chain variable region comprises a LCDR1, LCDR2, and LCDR3 from the light chain variable region of the same clone of Table 1. Also described is an antibody or antigen binding fragment comprising a heavy chain variable region and a light chain variable region, wherein the heavy chain variable region comprises a HCDR1, HCDR2, and HCDR3 having or having at least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a HCDR1, HCDR2, and HCDR3 from a heavy chain variable region of an antibody clone of Table 1 and wherein the light chain variable region comprises a LCDR1, LCDR2, and LCDR3 having or having at least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a LCDR1, LCDR2, and LCDR3 from the light chain variable region of the same clone of Table 1. The HCDR1, HCDR2, HCDR3, LCDR1, LCDR2, and/or LCDR3 may be determined from the variable region sequences by methods known in the art. The CDR may be a HCDR1, HCDR2, HCDR3, LCDR1, LCDR2, and/or LCDR3 determined by the Chothia method. The CDR may be a HCDR1, HCDR2, HCDR3, LCDR1, LCDR2, and/or LCDR3 determined by the Kabat method. The CDR may be a HCDR1, HCDR2, HCDR3, LCDR1, LCDR2, and/or LCDR3 determined by the IMGT method.

Also described is an antibody or antigen binding fragment in which the HCDR1, HCDR2, HCDR2, LCDR1, LCDR2, and LCDR3 each comprise an amino acid sequence that has at least 80% sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone. The HCDR1, HCDR2, HCDR2, LCDR1, LCDR2, and LCDR3 may each comprise an amino acid sequence that has or has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone. The HCDR1, HCDR2, HCDR2, LCDR1, LCDR2, and LCDR3 may each comprise the amino acid sequence of an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone.

Also described is an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the HCDR1, HCR2, HCR3 from a heavy chain variable region of a antibody clone of Table 1 and wherein the light chain variable region comprises a LCDR1, LCDR2, and LCDR3 having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LCDR1, LCDR2, and LCDR3 from the light chain variable region of the same antibody clone of Table 1. The antibody or antigen binding fragment may comprise a heavy chain variable region and a light chain variable region, wherein the heavy chain variable region comprises a HCDR1, HCDR2, and HCDR3 having or having at least 80% sequence identity to the HCDR1, HCR2, HCR3 from a heavy chain variable region of a antibody clone of Table 1 and wherein the light chain variable region comprises a LCDR1, LCDR2, and LCDR3 having at least 80% sequence identity to the LCDR1, LCDR2, and LCDR3 from the light chain variable region of the same antibody clone of Table 1. In some aspects, the heavy chain variable region comprises a HCDR1, HCDR2, and HCDR3 having the amino acid sequence of an of a HCDR1, HCDR2, and HCDR3 of a clone of Table 1 and the light chain variable region comprises a LCDR1, LCDR2, and LCDR3 comprising the amino acid sequence of the LCDR1, LCDR2, and LCDR3 from the light chain variable region of the same clone of Table 1.

The polypeptides of the disclosure may comprise at least two antigen binding fragments or antibodies, wherein each antigen binding fragment or antibody is independently selected from an antigen binding fragment or antibody of the disclosure, such as those disclosed in Table 1. The polypeptide may be multivalent. The polypeptide may be multispecific. The polypeptide may be bispecific. The polypeptide may comprise, comprise at least, or comprise at most 1, 2, 3, 4, 5, 6, 7, 8, 9, or 10 antigen binding regions or antibodies. Each antigen binding region or antibody may be independently selected from an antigen binding region or antibody of the disclosure, such as those in Table 1. The polypeptide may have repeated units of the same antigen binding region, such as at least, at most, or exactly 1, 2, 3, 4, 5, 6, 7, 8, 9, or 10 repeated units.

The heavy chain variable region may comprise an amino acid sequence with at least 80% sequence identity to a heavy chain variable region of an antibody clone of Table 1 and/or the light chain variable region may comprise an amino acid sequence with at least 80% sequence identity to the light chain variable region of the same antibody clone of Table 1. The heavy chain variable region may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain variable region of an antibody clone of Table 1 and/or the light chain variable region may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain variable region of the same antibody clone of Table 1. The heavy chain variable region may comprise the amino acid sequence of a heavy chain variable region of an antibody clone of Table 1 and/or the light chain variable region may comprise the amino acid sequence of the same antibody clone of Table 1. The antibody or antigen binding fragment may comprise a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence with at least 80% sequence identity to an HFR1, HFR2, HFR3, and HFR4, respectively, of an antibody clone of Table 1, and the LFR1, LFR2, LFR3, and LFR4 may comprise an amino acid sequence with at least 80% sequence identity to the LFR1, LFR2, LFR3, and LFR4, respectively, of the same antibody clone of Table 1. The antibody or antigen binding fragment may comprise a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to an HFR1, HFR2, HFR3, and HFR4, respectively, of an antibody clone of Table 1, and the LFR1, LFR2, LFR3, and LFR4 may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the LFR1, LFR2, LFR3, and LFR4, respectively, of the same antibody clone of Table 1. The HFR1, HFR2, HFR3, and HFR4 may comprise the amino acid sequence of an HFR1, HFR2, HFR3, and HFR4, respectively, of an antibody clone of Table 1, and the LFR1, LFR2, LFR3, and LFR4 may comprise the amino acid sequence of the LFR1, LFR2, LFR3, and LFR4, respectively, of the same antibody clone of Table 1. The antibody or antigen binding fragment may comprise a heavy chain and a light chain and wherein the heavy chain may comprise an amino acid sequence with at least 70% sequence identity to a heavy chain of an antibody clone of Table 1 and the light chain may comprise an amino acid sequence with at least 70% sequence identity to the light chain of the same antibody clone of Table 1. The antibody or antigen binding fragment may comprise a heavy chain and a light chain and wherein the heavy chain may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain of an antibody clone of Table 1 and the light chain may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain of the same antibody clone of Table 1. The antibody or antigen binding fragment may comprise a heavy chain and a light chain and wherein the heavy chain may comprise the amino acid sequence of an antibody clone of Table 1 and the light chain may comprise the amino acid sequence of the same antibody clone of Table 1.

The antibody or antigen binding fragment of the disclosure may be human, chimeric, or humanized. The antibody, or antigen binding fragment may bind a SARS-CoV-2 Spike, NP protein, or ORF8 with a K_{D} of about 10⁻⁶ nM to about 10⁻¹² pM. The antibody, or antigen binding fragment may bind a SARS-CoV-2 Spike, NP protein, or ORF8 with a K_{D} of about, a K_{D} of at least, or a K_{D} of at most 10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, or 10⁻¹⁸ (or any derivable range therein) μM, nM, or pM. The antibody or antigen binding fragment may specifically bind to a receptor-binding domain (RBD) of the SARS-CoV-2 spike protein. The antibody may be further defined as a neutralizing antibody. The antibody or antigen binding fragment may be further defined as a human antibody or antigen binding fragment, humanized antibody or antigen binding fragment, recombinant antibody or antigen binding fragment, chimeric antibody or antigen binding fragment, an antibody or antigen binding fragment derivative, a veneered antibody or antigen binding fragment, a diabody, a monoclonal antibody or antigen binding fragment, a single domain antibody, or a single chain antibody. The antigen binding fragment may be further defined as a single chain variable fragment (scFv), F(ab′)₂, Fab′, Fab, Fv, or rIgG. The antibody, antigen binding fragment, or polypeptide may be operatively linked to a detectable label. Detectable labels are described herein.

Also provided are multi-specific and/or multivalent antibodies and polypeptides. The disclosure provides for bivalent or bispecific antibodies that comprise two antigen binding fragments, wherein the antigen binding fragment is two of the same antigen binding fragments or two different antigen binding fragments described herein. The disclosure also provides for multi-specific polypeptides. The polypeptides may comprise at least 2, 3, 4, 5, or 6 antigen binding fragments.

The antigen binding fragment may be at least 2, 3, 4, 5, or 6 scFv, F(ab′)2, Fab′, Fab, Fv, or rIgG, or combinations thereof. The polypeptide and/or antigen binding fragments of the disclosure may comprise a linker between a heavy chain and light chain variable region or between antigen binding fragments. The linker may be a flexible linker. Exemplary flexible linkers include glycine polymers (G) n, glycine-serine polymers (including, for example, (GS)n, (GSGGS-SEQ ID NO: 1875)n, (G₄S)n and (GGGS-SEQ ID NO:1876) n, where n is an integer of at least one. n may be at least, at most, or exactly 1, 2, 3, 4, 5, 6, 7, 8, 9, or 10 (or any derivable range therein). Glycine-alanine polymers, alanine-serine polymers, and other flexible linkers known in the art and may be used as a linker in the polypeptides of the disclosure. Exemplary linkers can comprise or consist of GGSG (SEQ ID NO:1877), GGSGG (SEQ ID NO:1878), GSGSG (SEQ ID NO:1879), GSGGG (SEQ ID NO:1880), GGGSG (SEQ ID NO:1881), GSSSG (SEQ ID NO:1882), and the like.

The coronavirus infection may be a SARS-CoV-2 infection. The coronavirus infection may be a SARS-CoV infection. The coronavirus infection may be a MERS-CoV infection. The coronavirus infection may be a HCoV-OC43, HCoV-HKU1, HCOV-229E, or HCoV-NL63 infection.

Compositions of the disclosure, such as pharmaceutical compositions may comprise a pharmaceutical excipient, carrier, or molecule described herein. The composition may further comprises an adjuvant or an immunostimulator. Such adjuvants or immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments. Compositions may comprise more than one antibody and/or antigen binding fragment of the disclosure. Accordingly, compositions of the disclosure may comprise, may comprise at least, or may comprise at most 1, 2, 3, 4, 5, 6, 7, 8, 9, or 10 antibodies and/or antigen binding fragments of the disclosure, wherein each antibody or antigen binding fragment is independently selected from an antibody or antigen binding fragment of the disclosure, such as those shown in Table 1. The compositions of the disclosure may be formulated for a route of administration described herein. The composition, antibody, antigen binding fragment, or polypeptide may be formulated for parenteral, intravenous, subcutaneous, intramuscular, or intranasal administration. The compositions may be formulated for intranasal administration.

The polypeptides, compositions, antibodies, antigen binding fragments, nucleic acids, or host cells, when administered to a subject, may be provided or may be provided at least, or may be provided at most 1, 2, 3, 4, 5, 6, 7, 8, 9, 10, 11, 12, 13, 14, 15, 16, 17, 18, 19, or 20 times (or any derivable range therein) over the course of, over the course of at least, or over the course of at most 1, 2, 3, 4, 5, 6, 7, 8, 9, 10, 11, 12, 13, or 14 days or 1, 2, 3, 4, 5, 6, 7, 8 9, 10, 11, 12, 13, 14, 15, 16, 17, 18, 19, or 20 weeks, or 1, 2, 3, 4, 5, 6, 7, 8, 9, 10, 11, or 12 months, or 1, 2, 3, 4, 5, 6, 7, 8, 9, or 10 years (or any range derivable therein).

The host cell may be a human cell, B cell, T cell, Chinese hamster ovary, NS0 murine myeloma cell, or PER.C6 cell. The host cell may be a cell type or cell population described herein.

The subject or patient may be a human subject or a human patient. The subject or patient may be a non-human animal. The non-human animal may be a bat, monkey, camel, rat, mouse, rabbit, goat, chicken, bird, cat, or dog. The subject may further be defined as an at-risk subject. At-risk subjects include health care workers, immunocompromised subjects, people over the age of 65, or those with at least one or at least two underlying conditions. Example of underlying conditions include obesity, high blood pressure, autoimmunity, cancer, and asthma. The subject may be one that has one or more symptoms of a coronavirus infection. Symptoms of a coronavirus infection include, but are not limited to elevated temperature or a fever of 100.0° F. or more, loss of taste or smell, cough, difficulty breathing, shortness of breath, fatigue, headache, chills, sore throat, congestion or runny nose, shaking or exaggerated shivering, significant muscle pain or ache, diarrhea, and/or nausea or vomiting. The subject may be one that does not have any symptoms of a coronavirus infection. The subject may be one that has been diagnosed with a coronavirus infection. The subject may be one that has not been diagnosed with a coronavirus infection. The subject may be one that has been previously treated for a coronavirus infection. The subject may be one that has been previously vaccinated for coronavirus. The subject may be one that has not been previously vaccinated for coronavirus. The previous treatment may comprise a pain reliever, such as acetaminophen or ibuprofen, a steroid such as dexamethasone, prednisolone, beclomethasone, fluticasone, or methylprednisone or an antiviral such as remdesivir. The subject may be administered an additional therapeutic. The additional therapeutic may comprise one or more of a pain reliever, such as acetaminophen or ibuprofen, a steroid such as dexamethasone, prednisolone, beclomethasone, fluticasone, or methylprednisone or an antiviral such as remdesivir. The additional therapeutic may comprise dexamethasone. The additional therapeutic may comprise remdesivir.

The method may comprise or further comprise incubating the antibody, antigen binding fragment, or polypeptide under conditions that allow for the binding of the antibody, antigen binding fragment, or polypeptide to antigens in the biological sample or extract thereof. The method may comprise or further comprise detecting the binding of an antigen to the antibody, antigen binding fragment, or polypeptide. The method may comprise or further comprise contacting the biological sample with at least one capture antibody, antigen, or polypeptide. The at least one capture antibody, antigen binding fragment, or polypeptide may be an antibody, polypeptide, or antigen binding fragment of the disclosure. The capture antibody may be linked or operatively linked to a solid support. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. The biological sample may comprise a blood sample, urine sample, fecal sample, or nasopharyngeal sample. The at least one antibody, antigen binding fragment, or polypeptide may be operatively linked to a detectable label. The method may comprise or further comprise incubating the antibody, antigen binding fragment, or polypeptide under conditions that allow for the binding of the antibody, antigen binding fragment, or polypeptide to antigens in the biological sample or extract thereof. The method may comprise or further comprise detecting the binding of an antigen to the antibody, antigen binding fragment, or polypeptide. The method may comprise or further comprise contacting the biological sample with at least one capture antibody, antigen, or polypeptide. The biological sample may comprise a blood sample, urine sample, fecal sample, or nasopharyngeal sample.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 3, 4, and 5, respectively and the LCDR1, LCDR2, and LCDR3 comprises an amino acid sequence of SEQ ID NOS: 12, 13, and 14, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 21, 22, and 23, respectively and the LCDR1, LCDR2, and LCDR3 comprises an amino acid sequence of SEQ ID NOS: 29, 30, and 31, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 38, 39, and 40, respectively and the LCDR1, LCDR2, and LCDR3 comprises an amino acid sequence of SEQ ID NOS: 47, 48, and 49, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 56, 57, and 58, respectively and the LCDR1, LCDR2, and LCDR3 comprises an amino acid sequence of SEQ ID NOS: 63, 64, and 65, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 72, 73, and 74, respectively and the LCDR1, LCDR2, and LCDR3 comprises an amino acid sequence of SEQ ID NOS: 79, 80, and 81, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 3, 88, and 89, respectively and the LCDR1, LCDR2, and LCDR3 comprises an amino acid sequence of SEQ ID NOS: 63, 94, and 95, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 100, 101, and 102, respectively and the LCDR1, LCDR2, and LCDR3 comprises an amino acid sequence of SEQ ID NOS: 63, 64, and 65, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 56, 111, and 112, respectively and the LCDR1, LCDR2, and LCDR3 comprises an amino acid sequence of SEQ ID NOS: 118, 119, and 120, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 126, 127, and 128, respectively and the LCDR1, LCDR2, and LCDR3 comprises an amino acid sequence of SEQ ID NOS: 134, 135, and 136, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 141, 142, and 143, respectively and the LCDR1, LCDR2, and LCDR3 comprises an amino acid sequence of SEQ ID NOS: 150, 151, and 152, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 156, 157, and 158, respectively and the LCDR1, LCDR2, and LCDR3 comprises an amino acid sequence of SEQ ID NOS: 63, 164, and 165, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 170, 171, and 172, respectively and the LCDR1, LCDR2, and LCDR3 comprises an amino acid sequence of SEQ ID NOS: 178, 179, and 180, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 187, 188, and 189, respectively and the LCDR1, LCDR2, and LCDR3 comprises an amino acid sequence of SEQ ID NOS: 194, 135, and 195, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 199, 200, and 201, respectively and the LCDR1, LCDR2, and LCDR3 comprises an amino acid sequence of SEQ ID NOS: 207, 208, and 209, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 214, 215, and 216, respectively and the LCDR1, LCDR2, and LCDR3 comprises an amino acid sequence of SEQ ID NOS: 223, 224, and 225, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 231, 232, and 233, respectively and the LCDR1, LCDR2, and LCDR3 comprises an amino acid sequence of SEQ ID NOS: 237, 135, and 238, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 242, 243, and 244, respectively and the LCDR1, LCDR2, and LCDR3 comprises an amino acid sequence of SEQ ID NOS: 248, 249, and 250, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 199, 256, and 257, respectively and the LCDR1, LCDR2, and LCDR3 comprises an amino acid sequence of SEQ ID NOS: 262, 263, and 264, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 270, 271, and 272, respectively and the LCDR1, LCDR2, and LCDR3 comprises an amino acid sequence of SEQ ID NOS: 276, 277, and 278, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 284, 285, and 286, respectively and the LCDR1, LCDR2, and LCDR3 comprises an amino acid sequence of SEQ ID NOS: 291, 30, and 292, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 296, 297, and 298, respectively and the LCDR1, LCDR2, and LCDR3 comprises an amino acid sequence of SEQ ID NOS: 302, 135, and 303, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 308, 157, and 309, respectively and the LCDR1, LCDR2, and LCDR3 comprises an amino acid sequence of SEQ ID NOS: 313, 314, and 315, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 321, 322, and 323, respectively and the LCDR1, LCDR2, and LCDR3 comprises an amino acid sequence of SEQ ID NOS: 328, 249, and 329, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 56, 333, and 334, respectively and the LCDR1, LCDR2, and LCDR3 comprises an amino acid sequence of SEQ ID NOS: 338, 135, and 303, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 342, 343, and 344, respectively and the LCDR1, LCDR2, and LCDR3 comprises an amino acid sequence of SEQ ID NOS: 349, 350, and 351, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 358, 359, and 360, respectively and the LCDR1, LCDR2, and LCDR3 comprises an amino acid sequence of SEQ ID NOS: 365, 366, and 367, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 358, 373, and 374, respectively and the LCDR1, LCDR2, and LCDR3 comprises an amino acid sequence of SEQ ID NOS: 338, 135, and 378, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 242, 243, and 383, respectively and the LCDR1, LCDR2, and LCDR3 comprises an amino acid sequence of SEQ ID NOS: 386, 387, and 388, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 358, 392, and 393, respectively and the LCDR1, LCDR2, and LCDR3 comprises an amino acid sequence of SEQ ID NOS: 276, 396, and 397, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 56, 4, and 400, respectively and the LCDR1, LCDR2, and LCDR3 comprises an amino acid sequence of SEQ ID NOS: 403, 404, and 405, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 126, 409, and 410, respectively and the LCDR1, LCDR2, and LCDR3 comprises an amino acid sequence of SEQ ID NOS: 414, 13, and 415, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 199, 419, and 420, respectively and the LCDR1, LCDR2, and LCDR3 comprises an amino acid sequence of SEQ ID NOS: 207, 208, and 424, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 56, 427, and 428, respectively and the LCDR1, LCDR2, and LCDR3 comprises an amino acid sequence of SEQ ID NOS: 432, 249, and 433, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 437, 142, and 438, respectively and the LCDR1, LCDR2, and LCDR3 comprises an amino acid sequence of SEQ ID NOS: 178, 441, and 442, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 446, 447, and 448, respectively and the LCDR1, LCDR2, and LCDR3 comprises an amino acid sequence of SEQ ID NOS: 386, 387, and 452, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 457, 458, and 459, respectively and the LCDR1, LCDR2, and LCDR3 comprises an amino acid sequence of SEQ ID NOS: 464, 465, and 466, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 472, 473, and 474, respectively and the LCDR1, LCDR2, and LCDR3 comprises an amino acid sequence of SEQ ID NOS: 63, 64, and 480, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 56, 483, and 484, respectively and the LCDR1, LCDR2, and LCDR3 comprises an amino acid sequence of SEQ ID NOS: 489, 490, and 491, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 141, 497, and 498, respectively and the LCDR1, LCDR2, and LCDR3 comprises an amino acid sequence of SEQ ID NOS: 502, 503, and 504, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 141, 142, and 507, respectively and the LCDR1, LCDR2, and LCDR3 comprises an amino acid sequence of SEQ ID NOS: 502, 503, and 510, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 513, 514, and 515, respectively and the LCDR1, LCDR2, and LCDR3 comprises an amino acid sequence of SEQ ID NOS: 520, 521, and 522, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 527, 528, and 529, respectively and the LCDR1, LCDR2, and LCDR3 comprises an amino acid sequence of SEQ ID NOS: 535, 350, and 536, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 542, 543, and 544, respectively and the LCDR1, LCDR2, and LCDR3 comprises an amino acid sequence of SEQ ID NOS: 548, 387, and 549, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 141, 553, and 554, respectively and the LCDR1, LCDR2, and LCDR3 comprises an amino acid sequence of SEQ ID NOS: 262, 263, and 560, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 563, 564, and 565, respectively and the LCDR1, LCDR2, and LCDR3 comprises an amino acid sequence of SEQ ID NOS: 570, 249, and 571, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 187, 576, and 577, respectively and the LCDR1, LCDR2, and LCDR3 comprises an amino acid sequence of SEQ ID NOS: 403, 404, and 581, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 187, 585, and 586, respectively and the LCDR1, LCDR2, and LCDR3 comprises an amino acid sequence of SEQ ID NOS: 591, 592, and 593, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 599, 600, and 601, respectively and the LCDR1, LCDR2, and LCDR3 comprises an amino acid sequence of SEQ ID NOS: 605, 135, and 606, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 610, 611, and 612, respectively and the LCDR1, LCDR2, and LCDR3 comprises an amino acid sequence of SEQ ID NOS: 248, 249, and 617, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 622, 623, and 624, respectively and the LCDR1, LCDR2, and LCDR3 comprises an amino acid sequence of SEQ ID NOS: 630, 631, and 632, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 638, 639, and 640, respectively and the LCDR1, LCDR2, and LCDR3 comprises an amino acid sequence of SEQ ID NOS: 645, 387, and 646, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 650, 88, and 651, respectively and the LCDR1, LCDR2, and LCDR3 comprises an amino acid sequence of SEQ ID NOS: 262, 263, and 654, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 658, 543, and 659, respectively and the LCDR1, LCDR2, and LCDR3 comprises an amino acid sequence of SEQ ID NOS: 520, 387, and 646, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 187, 585, and 667, respectively and the LCDR1, LCDR2, and LCDR3 comprises an amino acid sequence of SEQ ID NOS: 673, 674, and 675, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 679, 680, and 681, respectively and the LCDR1, LCDR2, and LCDR3 comprises an amino acid sequence of SEQ ID NOS: 686, 687, and 688, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 693, 157, and 694, respectively and the LCDR1, LCDR2, and LCDR3 comprises an amino acid sequence of SEQ ID NOS: 520, 698, and 699, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 242, 243, and 704, respectively and the LCDR1, LCDR2, and LCDR3 comprises an amino acid sequence of SEQ ID NOS: 502, 366, and 708, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 527, 543, and 711, respectively and the LCDR1, LCDR2, and LCDR3 comprises an amino acid sequence of SEQ ID NOS: 673, 716, and 717, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 187, 724, and 725, respectively and the LCDR1, LCDR2, and LCDR3 comprises an amino acid sequence of SEQ ID NOS: 338, 135, and 728, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 734, 585, and 735, respectively and the LCDR1, LCDR2, and LCDR3 comprises an amino acid sequence of SEQ ID NOS: 740, 119, and 741, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 744, 543, and 745, respectively and the LCDR1, LCDR2, and LCDR3 comprises an amino acid sequence of SEQ ID NOS: 520, 387, and 750, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 754, 755, and 756, respectively and the LCDR1, LCDR2, and LCDR3 comprises an amino acid sequence of SEQ ID NOS: 761, 208, and 762, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 765, 497, and 766, respectively and the LCDR1, LCDR2, and LCDR3 comprises an amino acid sequence of SEQ ID NOS: 769, 770, and 771, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 775, 776, and 777, respectively and the LCDR1, LCDR2, and LCDR3 comprises an amino acid sequence of SEQ ID NOS: 782, 30, and 783, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 786, 787, and 788, respectively and the LCDR1, LCDR2, and LCDR3 comprises an amino acid sequence of SEQ ID NOS: 794, 30, and 795, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 798, 799, and 800, respectively and the LCDR1, LCDR2, and LCDR3 comprises an amino acid sequence of SEQ ID NOS: 804, 30, and 805, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 693, 809, and 810, respectively and the LCDR1, LCDR2, and LCDR3 comprises an amino acid sequence of SEQ ID NOS: 816, 135, and 817, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 56, 821, and 822, respectively and the LCDR1, LCDR2, and LCDR3 comprises an amino acid sequence of SEQ ID NOS: 403, 404, and 826, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 829, 830, and 831, respectively and the LCDR1, LCDR2, and LCDR3 comprises an amino acid sequence of SEQ ID NOS: 837, 64, and 838, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 842, 843, and 844, respectively and the LCDR1, LCDR2, and LCDR3 comprises an amino acid sequence of SEQ ID NOS: 520, 387, and 646, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 850, 851, and 852, respectively and the LCDR1, LCDR2, and LCDR3 comprises an amino acid sequence of SEQ ID NOS: 520, 387, and 856, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 860, 861, and 862, respectively and the LCDR1, LCDR2, and LCDR3 comprises an amino acid sequence of SEQ ID NOS: 867, 868, and 869, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 542, 875, and 876, respectively and the LCDR1, LCDR2, and LCDR3 comprises an amino acid sequence of SEQ ID NOS: 881, 387, and 882, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 888, 889, and 890, respectively and the LCDR1, LCDR2, and LCDR3 comprises an amino acid sequence of SEQ ID NOS: 895, 208, and 303, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 126, 899, and 900, respectively and the LCDR1, LCDR2, and LCDR3 comprises an amino acid sequence of SEQ ID NOS: 904, 905, and 906, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 610, 910, and 911, respectively and the LCDR1, LCDR2, and LCDR3 comprises an amino acid sequence of SEQ ID NOS: 645, 915, and 916, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 919, 920, and 921, respectively and the LCDR1, LCDR2, and LCDR3 comprises an amino acid sequence of SEQ ID NOS: 291, 30, and 924, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 929, 930, and 931, respectively and the LCDR1, LCDR2, and LCDR3 comprises an amino acid sequence of SEQ ID NOS: 935, 151, and 936, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 270, 940, and 941, respectively and the LCDR1, LCDR2, and LCDR3 comprises an amino acid sequence of SEQ ID NOS: 12, 947, and 948, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 187, 953, and 954, respectively and the LCDR1, LCDR2, and LCDR3 comprises an amino acid sequence of SEQ ID NOS: 63, 94, and 959, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 56, 4, and 963, respectively and the LCDR1, LCDR2, and LCDR3 comprises an amino acid sequence of SEQ ID NOS: 432, 249, and 966, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 929, 930, and 931, respectively and the LCDR1, LCDR2, and LCDR3 comprises an amino acid sequence of SEQ ID NOS: 935, 151, and 936, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 187, 953, and 970, respectively and the LCDR1, LCDR2, and LCDR3 comprises an amino acid sequence of SEQ ID NOS: 63, 974, and 975, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 979, 980, and 981, respectively and the LCDR1, LCDR2, and LCDR3 comprises an amino acid sequence of SEQ ID NOS: 63, 151, and 986, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 990, 991, and 992, respectively and the LCDR1, LCDR2, and LCDR3 comprises an amino acid sequence of SEQ ID NOS: 995, 770, and 996, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 308, 1000, and 1001, respectively and the LCDR1, LCDR2, and LCDR3 comprises an amino acid sequence of SEQ ID NOS: 63, 94, and 1005, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1009, 1010, and 1011, respectively and the LCDR1, LCDR2, and LCDR3 comprises an amino acid sequence of SEQ ID NOS: 1017, 64, and 1018, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 187, 1023, and 1024, respectively and the LCDR1, LCDR2, and LCDR3 comprises an amino acid sequence of SEQ ID NOS: 1028, 94, and 1029, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 56, 333, and 1032, respectively and the LCDR1, LCDR2, and LCDR3 comprises an amino acid sequence of SEQ ID NOS: 1036, 249, and 1037, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 187, 953, and 1042, respectively and the LCDR1, LCDR2, and LCDR3 comprises an amino acid sequence of SEQ ID NOS: 63, 64, and 1046, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1049, 1050, and 1051, respectively and the LCDR1, LCDR2, and LCDR3 comprises an amino acid sequence of SEQ ID NOS: 1058, 387, and 1059, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1062, 1063, and 1064, respectively and the LCDR1, LCDR2, and LCDR3 comprises an amino acid sequence of SEQ ID NOS: 1070, 441, and 1071, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 457, 458, and 1076, respectively and the LCDR1, LCDR2, and LCDR3 comprises an amino acid sequence of SEQ ID NOS: 1082, 208, and 1083, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 457, 1087, and 1088, respectively and the LCDR1, LCDR2, and LCDR3 comprises an amino acid sequence of SEQ ID NOS: 1091, 208, and 1092, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1062, 1095, and 1096, respectively and the LCDR1, LCDR2, and LCDR3 comprises an amino acid sequence of SEQ ID NOS: 1101, 1102, and 1103, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1109, 4, and 1110, respectively and the LCDR1, LCDR2, and LCDR3 comprises an amino acid sequence of SEQ ID NOS: 1115, 1116, and 1117, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1123, 1124, and 1125, respectively and the LCDR1, LCDR2, and LCDR3 comprises an amino acid sequence of SEQ ID NOS: 1130, 1131, and 1132, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 610, 1137, and 1138, respectively and the LCDR1, LCDR2, and LCDR3 comprises an amino acid sequence of SEQ ID NOS: 1142, 1143, and 1144, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 141, 142, and 1149, respectively and the LCDR1, LCDR2, and LCDR3 comprises an amino acid sequence of SEQ ID NOS: 1152, 249, and 1153, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1157, 1158, and 1159, respectively and the LCDR1, LCDR2, and LCDR3 comprises an amino acid sequence of SEQ ID NOS: 1165, 1166, and 1167, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1171, 1172, and 1173, respectively and the LCDR1, LCDR2, and LCDR3 comprises an amino acid sequence of SEQ ID NOS: 248, 249, and 1179, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1184, 1185, and 1186, respectively and the LCDR1, LCDR2, and LCDR3 comprises an amino acid sequence of SEQ ID NOS: 1191, 1192, and 1193, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 126, 127, and 1199, respectively and the LCDR1, LCDR2, and LCDR3 comprises an amino acid sequence of SEQ ID NOS: 338, 135, and 1202, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1205, 1206, and 1207, respectively and the LCDR1, LCDR2, and LCDR3 comprises an amino acid sequence of SEQ ID NOS: 338, 1211, and 1212, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1216, 1217, and 1218, respectively and the LCDR1, LCDR2, and LCDR3 comprises an amino acid sequence of SEQ ID NOS: 673, 350, and 1222, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1226, 1227, and 1228, respectively and the LCDR1, LCDR2, and LCDR3 comprises an amino acid sequence of SEQ ID NOS: 673, 1232, and 1222, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1235, 1236, and 1237, respectively and the LCDR1, LCDR2, and LCDR3 comprises an amino acid sequence of SEQ ID NOS: 1243, 592, and 1244, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 1249, 1250, and 1251, respectively and the LCDR1, LCDR2, and LCDR3 comprises an amino acid sequence of SEQ ID NOS: 570, 249, and 1255, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1258, 1259, and 1260, respectively and the LCDR1, LCDR2, and LCDR3 comprises an amino acid sequence of SEQ ID NOS: 769, 1265, and 1266, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1205, 1206, and 1207, respectively and the LCDR1, LCDR2, and LCDR3 comprises an amino acid sequence of SEQ ID NOS: 338, 1211, and 1212, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1272, 1273, and 1274, respectively and the LCDR1, LCDR2, and LCDR3 comprises an amino acid sequence of SEQ ID NOS: 63, 1280, and 1281, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1184, 1185, and 1186, respectively and the LCDR1, LCDR2, and LCDR3 comprises an amino acid sequence of SEQ ID NOS: 1191, 1192, and 1193, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 1216, 1217, and 1218, respectively and the LCDR1, LCDR2, and LCDR3 comprises an amino acid sequence of SEQ ID NOS: 673, 350, and 1222, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 72, 1286, and 1287, respectively and the LCDR1, LCDR2, and LCDR3 comprises an amino acid sequence of SEQ ID NOS: 1293, 30, and 1294, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1299, 1300, and 1301, respectively and the LCDR1, LCDR2, and LCDR3 comprises an amino acid sequence of SEQ ID NOS: 63, 151, and 1306, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1311, 4, and 1312, respectively and the LCDR1, LCDR2, and LCDR3 comprises an amino acid sequence of SEQ ID NOS: 816, 135, and 1318, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1226, 1227, and 1228, respectively and the LCDR1, LCDR2, and LCDR3 comprises an amino acid sequence of SEQ ID NOS: 673, 1232, and 1222, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 1258, 1259, and 1260, respectively and the LCDR1, LCDR2, and LCDR3 comprises an amino acid sequence of SEQ ID NOS: 769, 1265, and 1266, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1321, 1322, and 1323, respectively and the LCDR1, LCDR2, and LCDR3 comprises an amino acid sequence of SEQ ID NOS: 1329, 1330, and 1331, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1321, 1322, and 1323, respectively and the LCDR1, LCDR2, and LCDR3 comprises an amino acid sequence of SEQ ID NOS: 1329, 1330, and 1331, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1272, 1273, and 1274, respectively and the LCDR1, LCDR2, and LCDR3 comprises an amino acid sequence of SEQ ID NOS: 63, 1280, and 1281, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 72, 1286, and 1287, respectively and the LCDR1, LCDR2, and LCDR3 comprises an amino acid sequence of SEQ ID NOS: 1293, 30, and 1294, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 1335, 1336, and 1337, respectively and the LCDR1, LCDR2, and LCDR3 comprises an amino acid sequence of SEQ ID NOS: 1342, 1343, and 1344, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1350, 1351, and 1352, respectively and the LCDR1, LCDR2, and LCDR3 comprises an amino acid sequence of SEQ ID NOS: 1359, 30, and 1360, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 187, 585, and 1363, respectively and the LCDR1, LCDR2, and LCDR3 comprises an amino acid sequence of SEQ ID NOS: 63, 151, and 1366, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1369, 1370, and 1371, respectively and the LCDR1, LCDR2, and LCDR3 comprises an amino acid sequence of SEQ ID NOS: 338, 135, and 1375, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 187, 585, and 1363, respectively and the LCDR1, LCDR2, and LCDR3 comprises an amino acid sequence of SEQ ID NOS: 63, 151, and 1366, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 1378, 1379, and 1380, respectively and the LCDR1, LCDR2, and LCDR3 comprises an amino acid sequence of SEQ ID NOS: 1385, 441, and 1386, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1389, 953, and 1390, respectively and the LCDR1, LCDR2, and LCDR3 comprises an amino acid sequence of SEQ ID NOS: 1395, 1396, and 1397, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1369, 1370, and 1371, respectively and the LCDR1, LCDR2, and LCDR3 comprises an amino acid sequence of SEQ ID NOS: 338, 135, and 1375, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 141, 142, and 1149, respectively and the LCDR1, LCDR2, and LCDR3 comprises an amino acid sequence of SEQ ID NOS: 1152, 249, and 1153, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1402, 1403, and 1404, respectively and the LCDR1, LCDR2, and LCDR3 comprises an amino acid sequence of SEQ ID NOS: 1036, 249, and 1408, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 1378, 1379, and 1380, respectively and the LCDR1, LCDR2, and LCDR3 comprises an amino acid sequence of SEQ ID NOS: 1385, 441, and 1386, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1184, 1185, and 1186, respectively and the LCDR1, LCDR2, and LCDR3 comprises an amino acid sequence of SEQ ID NOS: 1191, 1192, and 1193, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1389, 953, and 1390, respectively and the LCDR1, LCDR2, and LCDR3 comprises an amino acid sequence of SEQ ID NOS: 1395, 1396, and 1397, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1412, 1413, and 1414, respectively and the LCDR1, LCDR2, and LCDR3 comprises an amino acid sequence of SEQ ID NOS: 1395, 94, and 1419, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 199, 1422, and 1423, respectively and the LCDR1, LCDR2, and LCDR3 comprises an amino acid sequence of SEQ ID NOS: 1428, 1429, and 1430, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 798, 1436, and 1437, respectively and the LCDR1, LCDR2, and LCDR3 comprises an amino acid sequence of SEQ ID NOS: 1442, 208, and 1443, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1447, 473, and 1448, respectively and the LCDR1, LCDR2, and LCDR3 comprises an amino acid sequence of SEQ ID NOS: 63, 151, and 1454, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1457, 1458, and 1459, respectively and the LCDR1, LCDR2, and LCDR3 comprises an amino acid sequence of SEQ ID NOS: 1464, 1465, and 1466, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1472, 1473, and 1474, respectively and the LCDR1, LCDR2, and LCDR3 comprises an amino acid sequence of SEQ ID NOS: 1478, 119, and 1479, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1482, 1483, and 1484, respectively and the LCDR1, LCDR2, and LCDR3 comprises an amino acid sequence of SEQ ID NOS: 338, 1143, and 1488, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 1492, 243, and 1493, respectively and the LCDR1, LCDR2, and LCDR3 comprises an amino acid sequence of SEQ ID NOS: 1497, 1498, and 1499, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1505, 953, and 1506, respectively and the LCDR1, LCDR2, and LCDR3 comprises an amino acid sequence of SEQ ID NOS: 63, 592, and 1511, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1515, 1516, and 1517, respectively and the LCDR1, LCDR2, and LCDR3 comprises an amino acid sequence of SEQ ID NOS: 1522, 1523, and 1524, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1235, 1529, and 1530, respectively and the LCDR1, LCDR2, and LCDR3 comprises an amino acid sequence of SEQ ID NOS: 1535, 1536, and 1537, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 358, 392, and 1543, respectively and the LCDR1, LCDR2, and LCDR3 comprises an amino acid sequence of SEQ ID NOS: 1547, 770, and 1548, respectively.

The disclosure describes an antibody, antigen binding fragment, or polypeptide comprising a heavy chain variable region having a HCDR1, HCDR2, and HCDR3, and a light HCDR3 comprises an amino acid sequence of SEQ ID NOS: 1551, 1552, and 1553, respectively and the LCDR1, LCDR2, and LCDR3 comprises an amino acid sequence of SEQ ID NOS: 1558, 249, and 1559, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1565, 1566, and 1567, respectively and the LCDR1, LCDR2, and LCDR3 comprises an amino acid sequence of SEQ ID NOS: 1572, 1573, and 1574, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1579, 1580, and 1581, respectively and the LCDR1, LCDR2, and LCDR3 comprises an amino acid sequence of SEQ ID NOS: 1585, 135, and 1586, respectively.

The disclosure describes an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 1590, 1591, and 1592, respectively and the LCDR1, LCDR2, and LCDR3 comprises an amino acid sequence of SEQ ID NOS: 1599, 135, and 1600, respectively.

The disclosure also describes a heavy and light chain comprising the sequences of SEQ ID NO: 1 and SEQ ID NO: 10; SEQ ID NO:19 and SEQ ID NO:27; SEQ ID NO:36 and SEQ ID NO: 45; SEQ ID NO:54 and SEQ ID NO:61; SEQ ID NO: 70 and SEQ ID NO:77; SEQ ID NO:86 and SEQ ID NO:92; SEQ ID NO:98 and SEQ ID NO: 107; SEQ ID NO: 109 and SEQ ID NO: 116; SEQ ID NO: 124 and SEQ ID NO: 132; SEQ ID NO:139 and SEQ ID NO:148; SEQ ID NO:154 and SEQ ID NO:162; SEQ ID NO:168 and SEQ ID NO:176; SEQ ID NO: 185 and SEQ ID NO: 192; SEQ ID NO: 197 and SEQ ID NO:205; SEQ ID NO:212 and SEQ ID NO:221; SEQ ID NO: 229 and SEQ ID NO:235; SEQ ID NO:240 and SEQ ID NO:246; SEQ ID NO:254 and SEQ ID NO: 260; SEQ ID NO:268 and SEQ ID NO:274; SEQ ID NO:282 and SEQ ID NO:289; SEQ ID NO: 294 and SEQ ID NO:300; SEQ ID NO:306 and SEQ ID NO:311; SEQ ID NO:319 and SEQ ID NO:326; SEQ ID NO:331 and SEQ ID NO:336; SEQ ID NO:340 and SEQ ID NO:347; SEQ ID NO:356 and SEQ ID NO:363; SEQ ID NO:371 and SEQ ID NO:376; SEQ ID NO:381 and SEQ ID NO:384; SEQ ID NO:390 and SEQ ID NO:394; SEQ ID NO:398 and SEQ ID NO: 401; SEQ ID NO:407 and SEQ ID NO:412; SEQ ID NO:417 and SEQ ID NO:422; SEQ ID NO: 425 and SEQ ID NO:430; SEQ ID NO: 435 and SEQ ID NO: 439; SEQ ID NO:444 and SEQ ID NO: 450; SEQ ID NO:455 and SEQ ID NO:462; SEQ ID NO:470 and SEQ ID NO:478; SEQ ID NO: 481 and SEQ ID NO:487; SEQ ID NO:495 and SEQ ID NO:500; SEQ ID NO:505 and SEQ ID NO:508; SEQ ID NO:511 and SEQ ID NO:518; SEQ ID NO:525 and SEQ ID NO:533; SEQ ID NO:540 and SEQ ID NO:546; SEQ ID NO:551 and SEQ ID NO:558; SEQ ID NO:561 and SEQ ID NO:568; SEQ ID NO:574 and SEQ ID NO:579; SEQ ID NO:583 and SEQ ID NO: 589; SEQ ID NO:597 and SEQ ID NO:603; SEQ ID NO:608 and SEQ ID NO:615; SEQ ID NO: 620 and SEQ ID NO: 628; SEQ ID NO:636 and SEQ ID NO:643; SEQ ID NO:648 and SEQ ID NO: 652; SEQ ID NO:656 and SEQ ID NO:662; SEQ ID NO:665 and SEQ ID NO:671; SEQ ID NO: 677 and SEQ ID NO:684; SEQ ID NO:691 and SEQ ID NO:696; SEQ ID NO: 702 and SEQ ID NO:706; SEQ ID NO:709 and SEQ ID NO:714; SEQ ID NO:722 and SEQ ID NO:726; SEQ ID NO: 732 and SEQ ID NO: 738; SEQ ID NO: 742 and SEQ ID NO:748; SEQ ID NO:752 and SEQ ID NO:759; SEQ ID NO:763 and SEQ ID NO:767; SEQ ID NO:773 and SEQ ID NO: 780; SEQ ID NO: 784 and SEQ ID NO: 792; SEQ ID NO: 796 and SEQ ID NO:802; SEQ ID NO: 807 and SEQ ID NO:814; SEQ ID NO:819 and SEQ ID NO: 824; SEQ ID NO:827 and SEQ ID NO: 835; SEQ ID NO:840 and SEQ ID NO:846; SEQ ID NO:848 and SEQ ID NO:854; SEQ ID NO: 858 and SEQ ID NO:865; SEQ ID NO:873 and SEQ ID NO:879; SEQ ID NO:886 and SEQ ID NO:893; SEQ ID NO:897 and SEQ ID NO:902; SEQ ID NO:908 and SEQ ID NO:913; SEQ ID NO:917 and SEQ ID NO:922; SEQ ID NO:927 and SEQ ID NO:933; SEQ ID NO:938 and SEQ ID NO:945; SEQ ID NO:951 and SEQ ID NO:957; SEQ ID NO:961 and SEQ ID NO: 964; SEQ ID NO: 927 and SEQ ID NO:933; SEQ ID NO:968 and SEQ ID NO:972; SEQ ID NO: 977 and SEQ ID NO: 984; SEQ ID NO: 988 and SEQ ID NO: 993; SEQ ID NO:998 and SEQ ID NO: 1003; SEQ ID NO: 1007 and SEQ ID NO:1015; SEQ ID NO:1021 and SEQ ID NO:1026; SEQ ID NO:1030 and SEQ ID NO:1034; SEQ ID NO: 1040 and SEQ ID NO: 1044; SEQ ID NO: 1047 and SEQ ID NO: 1056; SEQ ID NO: 1060 and SEQ ID NO: 1068; SEQ ID NO: 1074 and SEQ ID NO:1080; SEQ ID NO: 1085 and SEQ ID NO: 1089; SEQ ID NO: 1093 and SEQ ID NO: 1099; SEQ ID NO: 1107 and SEQ ID NO: 1113; SEQ ID NO: 1121 and SEQ ID NO:1128; SEQ ID NO:1135 and SEQ ID NO:1140; SEQ ID NO:1147 and SEQ ID NO: 1150; SEQ ID NO: 1155 and SEQ ID NO:1163; SEQ ID NO:1169 and SEQ ID NO: 1177; SEQ ID NO: 1182 and SEQ ID NO: 1189; SEQ ID NO:1197 and SEQ ID NO: 1200; SEQ ID NO:1203 and SEQ ID NO: 1209; SEQ ID NO: 1214 and SEQ ID NO: 1220; SEQ ID NO: 1224 and SEQ ID NO: 1230; SEQ ID NO: 1233 and SEQ ID NO: 1241; SEQ ID NO:1247 and SEQ ID NO: 1253; SEQ ID NO: 1256 and SEQ ID NO: 1263; SEQ ID NO: 1203 and SEQ ID NO: 1209; SEQ ID NO: 1270 and SEQ ID NO:1278; SEQ ID NO: 1182 and SEQ ID NO:1189; SEQ ID NO: 1214 and SEQ ID NO: 1220; SEQ ID NO: 1284 and SEQ ID NO: 1291; SEQ ID NO: 1297 and SEQ ID NO: 1304; SEQ ID NO:1309 and SEQ ID NO:1316; SEQ ID NO:1224 and SEQ ID NO: 1230; SEQ ID NO: 1256 and SEQ ID NO: 1263; SEQ ID NO: 1319 and SEQ ID NO: 1327; SEQ ID NO: 1319 and SEQ ID NO:1327; SEQ ID NO:1270 and SEQ ID NO:1278; SEQ ID NO:1284 and SEQ ID NO: 1291; SEQ ID NO: 1333 and SEQ ID NO: 1340; SEQ ID NO: 1348 and SEQ ID NO: 1357; SEQ ID NO:1361 and SEQ ID NO: 1364; SEQ ID NO:1367 and SEQ ID NO: 1374; SEQ ID NO: 1361 and SEQ ID NO: 1364; SEQ ID NO: 1376 and SEQ ID NO: 1383; SEQ ID NO: 1387 and SEQ ID NO: 1393; SEQ ID NO: 1367 and SEQ ID NO: 1374; SEQ ID NO:1147 and SEQ ID NO: 1150; SEQ ID NO:1400 and SEQ ID NO:1406; SEQ ID NO:1376 and SEQ ID NO:1383; SEQ ID NO:1182 and SEQ ID NO:1189; SEQ ID NO: 1387 and SEQ ID NO: 1393; SEQ ID NO: 1410 and SEQ ID NO: 1417; SEQ ID NO: 1420 and SEQ ID NO: 1426; SEQ ID NO: 1434 and SEQ ID NO: 1440; SEQ ID NO:1445 and SEQ ID NO:1452; SEQ ID NO: 1455 and SEQ ID NO: 1462; SEQ ID NO: 1470 and SEQ ID NO: 1476; SEQ ID NO: 1480 and SEQ ID NO: 1486; SEQ ID NO: 1490 and SEQ ID NO: 1495; SEQ ID NO: 1503 and SEQ ID NO: 1509; SEQ ID NO: 1513 and SEQ ID NO: 1520; SEQ ID NO: 1527 and SEQ ID NO: 1533; SEQ ID NO: 1541 and SEQ ID NO: 1545; SEQ ID NO: 1549 and SEQ ID NO:1556; SEQ ID NO: 1563 and SEQ ID NO: 1570; SEQ ID NO: 1577 and SEQ ID NO: 1583; or SEQ ID NO: 1588 and SEQ ID NO: 1597.

The disclosure also describes a heavy and light chain comprising the sequences of SEQ ID NO: 2 and SEQ ID NO: 11; SEQ ID NO:20 and SEQ ID NO:28; SEQ ID NO:37 and SEQ ID NO: 46; SEQ ID NO:55 and SEQ ID NO:62; SEQ ID NO: 71 and SEQ ID NO: 78; SEQ ID NO:87 and SEQ ID NO: 93; SEQ ID NO:99 and SEQ ID NO: 108; SEQ ID NO: 110 and SEQ ID NO: 117; SEQ ID NO: 125 and SEQ ID NO: 133; SEQ ID NO: 140 and SEQ ID NO:149; SEQ ID NO: 155 and SEQ ID NO:163; SEQ ID NO:169 and SEQ ID NO:177; SEQ ID NO: 186 and SEQ ID NO: 193; SEQ ID NO: 198 and SEQ ID NO:206; SEQ ID NO:213 and SEQ ID NO:222; SEQ ID NO: 230 and SEQ ID NO:236; SEQ ID NO:241 and SEQ ID NO:247; SEQ ID NO:255 and SEQ ID NO: 261; SEQ ID NO:269 and SEQ ID NO:275; SEQ ID NO:283 and SEQ ID NO:290; SEQ ID NO: 295 and SEQ ID NO:301; SEQ ID NO:307 and SEQ ID NO:312; SEQ ID NO:320 and SEQ ID NO:327; SEQ ID NO:332 and SEQ ID NO:337; SEQ ID NO:341 and SEQ ID NO:348; SEQ ID NO:357 and SEQ ID NO:364; SEQ ID NO:372 and SEQ ID NO:377; SEQ ID NO:382 and SEQ ID NO:385; SEQ ID NO:391 and SEQ ID NO:395; SEQ ID NO:399 and SEQ ID NO: 402; SEQ ID NO:408 and SEQ ID NO:413; SEQ ID NO:418 and SEQ ID NO:423; SEQ ID NO: 426 and SEQ ID NO:431; SEQ ID NO:436 and SEQ ID NO:440; SEQ ID NO:445 and SEQ ID NO: 451; SEQ ID NO:456 and SEQ ID NO:463; SEQ ID NO:471 and SEQ ID NO:479; SEQ ID NO: 482 and SEQ ID NO:488; SEQ ID NO:496 and SEQ ID NO:501; SEQ ID NO:506 and SEQ ID NO:509; SEQ ID NO:512 and SEQ ID NO:519; SEQ ID NO:526 and SEQ ID NO:534; SEQ ID NO:541 and SEQ ID NO:547; SEQ ID NO:552 and SEQ ID NO:559; SEQ ID NO:562 and SEQ ID NO:569; SEQ ID NO:575 and SEQ ID NO:580; SEQ ID NO:584 and SEQ ID NO: 590; SEQ ID NO:598 and SEQ ID NO:604; SEQ ID NO:609 and SEQ ID NO:616; SEQ ID NO: 621 and SEQ ID NO: 629; SEQ ID NO: 637 and SEQ ID NO: 644; SEQ ID NO:649 and SEQ ID NO: 653; SEQ ID NO:657 and SEQ ID NO:663; SEQ ID NO:666 and SEQ ID NO:672; SEQ ID NO: 678 and SEQ ID NO:685; SEQ ID NO: 692 and SEQ ID NO:697; SEQ ID NO:703 and SEQ ID NO: 707; SEQ ID NO:710 and SEQ ID NO:715; SEQ ID NO:723 and SEQ ID NO:727; SEQ ID NO: 733 and SEQ ID NO: 739; SEQ ID NO: 743 and SEQ ID NO:749; SEQ ID NO: 753 and SEQ ID NO:760; SEQ ID NO:764 and SEQ ID NO: 768; SEQ ID NO:774 and SEQ ID NO: 781; SEQ ID NO: 785 and SEQ ID NO: 793; SEQ ID NO: 797 and SEQ ID NO:803; SEQ ID NO: 808 and SEQ ID NO:815; SEQ ID NO: 820 and SEQ ID NO:825; SEQ ID NO:828 and SEQ ID NO: 836; SEQ ID NO:841 and SEQ ID NO:847; SEQ ID NO:849 and SEQ ID NO:855; SEQ ID NO: 859 and SEQ ID NO:866; SEQ ID NO:874 and SEQ ID NO:880; SEQ ID NO:887 and SEQ ID NO:894; SEQ ID NO: 898 and SEQ ID NO: 903; SEQ ID NO:909 and SEQ ID NO:914; SEQ ID NO:918 and SEQ ID NO:923; SEQ ID NO: 928 and SEQ ID NO:934; SEQ ID NO:939 and SEQ ID NO:946; SEQ ID NO:952 and SEQ ID NO:958; SEQ ID NO:962 and SEQ ID NO: 965; SEQ ID NO:928 and SEQ ID NO: 934; SEQ ID NO: 969 and SEQ ID NO:973; SEQ ID NO: 978 and SEQ ID NO: 985; SEQ ID NO:989 and SEQ ID NO: 994; SEQ ID NO: 999 and SEQ ID NO: 1004; SEQ ID NO:1008 and SEQ ID NO:1016; SEQ ID NO: 1022 and SEQ ID NO:1027; SEQ ID NO: 1031 and SEQ ID NO: 1035; SEQ ID NO: 1041 and SEQ ID NO: 1045; SEQ ID NO: 1048 and SEQ ID NO: 1057; SEQ ID NO: 1061 and SEQ ID NO: 1069; SEQ ID NO: 1075 and SEQ ID NO:1081; SEQ ID NO:1086 and SEQ ID NO:1090; SEQ ID NO:1094 and SEQ ID NO: 1100; SEQ ID NO: 1108 and SEQ ID NO:1114; SEQ ID NO: 1122 and SEQ ID NO:1129; SEQ ID NO:1136 and SEQ ID NO:1141; SEQ ID NO: 1148 and SEQ ID NO:1151; SEQ ID NO: 1156 and SEQ ID NO: 1164; SEQ ID NO: 1170 and SEQ ID NO: 1178; SEQ ID NO: 1183 and SEQ ID NO:1190; SEQ ID NO:1198 and SEQ ID NO: 1201; SEQ ID NO: 1204 and SEQ ID NO: 1210; SEQ ID NO: 1215 and SEQ ID NO: 1221; SEQ ID NO: 1225 and SEQ ID NO: 1231; SEQ ID NO:1234 and SEQ ID NO: 1242; SEQ ID NO: 1248 and SEQ ID NO: 1254; SEQ ID NO: 1257 and SEQ ID NO: 1264; SEQ ID NO: 1204 and SEQ ID NO: 1210; SEQ ID NO: 1271 and SEQ ID NO:1279; SEQ ID NO:1183 and SEQ ID NO: 1190; SEQ ID NO: 1215 and SEQ ID NO: 1221; SEQ ID NO: 1285 and SEQ ID NO: 1292; SEQ ID NO: 1298 and SEQ ID NO: 1305; SEQ ID NO:1310 and SEQ ID NO:1317; SEQ ID NO:1225 and SEQ ID NO: 1231; SEQ ID NO: 1257 and SEQ ID NO: 1264; SEQ ID NO: 1320 and SEQ ID NO: 1328; SEQ ID NO: 1320 and SEQ ID NO:1328; SEQ ID NO: 1271 and SEQ ID NO: 1279; SEQ ID NO: 1285 and SEQ ID NO: 1292; SEQ ID NO: 1334 and SEQ ID NO: 1341; SEQ ID NO: 1349 and SEQ ID NO: 1358; SEQ ID NO:1362 and SEQ ID NO:1365; SEQ ID NO: 1368 and SEQ ID NO: 1201; SEQ ID NO: 1362 and SEQ ID NO: 1365; SEQ ID NO: 1377 and SEQ ID NO: 1384; SEQ ID NO: 1388 and SEQ ID NO:1394; SEQ ID NO:1368 and SEQ ID NO: 1201; SEQ ID NO: 1148 and SEQ ID NO: 1151; SEQ ID NO:1401 and SEQ ID NO:1407; SEQ ID NO:1377 and SEQ ID NO:1384; SEQ ID NO:1183 and SEQ ID NO:1190; SEQ ID NO: 1388 and SEQ ID NO:1394; SEQ ID NO: 1411 and SEQ ID NO: 1418; SEQ ID NO: 1421 and SEQ ID NO: 1427; SEQ ID NO: 1435 and SEQ ID NO:1441; SEQ ID NO: 1446 and SEQ ID NO:1453; SEQ ID NO: 1456 and SEQ ID NO: 1463; SEQ ID NO: 1471 and SEQ ID NO: 1477; SEQ ID NO: 1481 and SEQ ID NO: 1487; SEQ ID NO:1491 and SEQ ID NO: 1496; SEQ ID NO: 1504 and SEQ ID NO: 1510; SEQ ID NO: 1514 and SEQ ID NO: 1521; SEQ ID NO: 1528 and SEQ ID NO: 1534; SEQ ID NO: 1542 and SEQ ID NO:1546; SEQ ID NO:1550 and SEQ ID NO:1557; SEQ ID NO:1564 and SEQ ID NO: 1571; SEQ ID NO: 1578 and SEQ ID NO: 1584; or SEQ ID NO: 1589 and SEQ ID NO: 1598.

“Treatment” or treating may refer to any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. The treatment may exclude prevention of the disease.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary, Detailed Description, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C. Analyses of serum antibody responses in COVID-19 convalescent individuals. a, b, Total IgG endpoint antibody titers from 10 convalescent subjects against SARS-CoV-2 full-length spike variants (a) and RBD recombinant antigens (b). Dashed line is the mean IgG titer. c, Neutralization titers from 10 convalescent donors against WT SARS-CoV-2, B.1.1.7, P.1, B.1.617.2 and B.1.617.1. Dashed line represents the mean neutralization titer. Data in a-c were analyzed using non-parametric Friedman's test with Dunnett's multiple comparison test. Fold-change in relative mAb binding to variants or mutants compared to WT in a and b are indicated above the statistical asterisks.

FIGS. 2A-I. a, b, Uniform manifold approximation and projection (UMAP) of SARS-CoV-2 spike non-RBD binding (a) and spike RBD binding B cells (b) isolated from the PBMCs of 10 convalescent subjects. c, The proportion of spike non-RBD and spike RBD specific binding B cells. The number in center of pie chart indicates the number of antigen-specific binding B cells. d, mAbs generated from selected B cells (n=43) were tested for binding to full-length spike, S1, S2, and RBD and neutralization potential against WT SARS-CoV-2. Binding data are represented as area under the curve (AUC). Neutralizing activity less than 10,000 ng/ml are considered neutralizing. e, f, Pie charts of mAbs domain specificity (e) and neutralizing capability (f). Number in the center of pie graphs indicate the number of antibodies tested. g, Comparison of neutralizing capability of mAbs targeting spike RBD and spike non-RBD. h and i, IC50 of neutralization potency of spike-reactive antibodies against WT virus based on domain specificity (h) and by subject (i). Mean in h indicated as a solid line. Dashed lines shown in h and i indicate limit of detection (10,000 ng/mL). Data in d-i are representative of two independent experiment performed in duplicate. Genetic characterization of each mAb is further detailed in Extended Data Table 2 (Example 1).

FIG. 3A-H. Binding breadth and neutralization of spike non-RBD mAbs. a, Full-length spike protein binding to ACE2 (a; PDB: 7KJ2). b-g, Locations of mutations found on B.1.1.7 (b), B.1.351 (c), P.1 (d), B.1.617.2 (e), B.1.526 (f) and B.1.617.1 (g). (b-g; modified from PDB: 6XM4). h, The binding reactivity and neutralization capabilities of NTA-A, NTD-B and S2 reactive mAbs. The color gradients indicate percentage of relative binding compared to WT spike. The neutralization potency (IC50) of spike-non RBD mAbs against WT, B.1.1.7, P.1, B.1.617.2 and B.1.617.1 variants are indicated as ng/ml. The panel of SARS-CoV-2 viruses are detailed in Extended Data Table 4 (Example 1). Data in h are representative of two independent experiments performed in duplicate. Genetic information for each mAb is in Extended Data Table 2 (Example 1).

FIG. 4A-C. Binding and neutralization profiles of RBD-binding 2 mAbs against a panel of RBD escape mutants and variants. a, Structural model of RBD “up” binding with ACE2 (a; PDB: 7KJ2) and RBD antibody classes and associated escape mutants. b, RBD is colored by antibody classes and associated mutations. c, Heatmap detailing binding reactivity of RBD mAbs (n=29) against single key escape sites for class 1, class 2 and class 3 antibodies, combinations of RBD mutants, and RBD from SARS-CoV-1 and MERS-CoV. Abbreviation of a refers to class-3 like antibodies, which are defined by mAbs that compete with a class 3 mAb (Extended Data FIG. 2c). Abbreviations b-f refer to mutations in the RBD of each full length spike variant, B.1.1.7 with N501Y (b), B.1.351 with K417N:E484K:N501Y (c), P.1 with K417T:E484K:N501Y (d), B.1.617.2 with T478K: L452R (e), B.1.526 with E484K (f) and B.1.617.1 with L452R:E484Q (g). The panel of recombinant antigens in c are detailed in Extended Data Table 3 (Example 1), including mutations found in circulating SARS-CoV-2 variants (bold), the mutations that escape/reduce binding by polyclonal serum/potent neutralizing mAbs (italic), the mutations found in both circulating SARS-CoV-2 variants and in vitro escape-map (bold+italic), and artificial mutants at key contact residues of the RBD-ACE2 interaction (normal typeface). The neutralization potency (IC50) of spike-RBD mAbs against WT, B.1.1.7, P.1, B.1.617.2 and B.1.617.1 variants are indicated as ng/ml. The panel of SARS-CoV-2 viruses are detailed in Extended Data Table 4 (Example 1). Data in c are representative of two independent experiments performed in duplicate. Genetic information for each antibody is in Extended Data Table 2 (Example 1).

FIG. 5A-I. MAb genetic, somatic hypermutation, and CDR3 length features. a-d, The distribution of V gene usage of spike-non RBD and spike RBD antibodies for all paired heavy (a, c) and light (b, d) chains. Percentage shown indicates proportion of the top 3 utilized genes. e, Clonal relationships between heavy and light chain variable gene locus of spike non-RBD and spike RBD-specific antibodies. Connecting lines represent the pairing of heavy and light chain of antibody clones specific to spike non-RBD or RBD and antibody clones shared between both groups (purple). f, g, Comparison of number of somatic hypermutations of heavy (f) and light chains (g) of spike non-RBD and spike RBD-binding B cells. h and i, The complementarity determining region 3 (CDR3) amino acid length for heavy (h) and light chains (i) of spike non-RBD and spike RBD-binding B cells. Median indicated as line in the box and whisker graph. Each dot represents an individual antibody with range from minimum to maximum value. Data in f-i were analyzed using Mann-Whitney non-parametric test.

FIG. 6A-E. MAb binding competition by ELISA and BLI and serum competition by ELISA. a, Competition ELISA of RBD mAbs of spike non-RBD mAbs with NTD-A (S451-11) and NTD-B (S305-1456). b, Competition ELISA of RBD mAbs of undetermined class with class 2 mAbs (S144-1079 and S564-138) and class 3 mAb (S24-821). c, MAb binding competition by BLI of class 2 mAb, S144-1406, with the other class 2 mAbs (n=4) that did not neutralize P.1. d, MAb binding competition by BLI between class 4 mAbs that utilized VH5-51 (S144-466, S144-509, S144 and S144-69) with CR3022. e, EC50 of serum antibodies of 10 convalescent subjects competing with RBD-reactive mAbs for binding to RBD class 2, class 3 and class 3-like epitopes, and NTD-reactive mAbs for binding to NTD-B epitopes. Dashed line represents the limit of detection. Data in a-b and e are representative of two independent experiments performed in duplicate. Data in e were analyzed using nonparametric Friedman's test with Dunn's multiple comparison test.

FIG. 7A-B. Comparison of neutralization potency of SARS-CoV-2 neutralizing mAbs. a, Neutralization potency (IC50) of RBD-binding mAbs, class 2 and class 3, and NTD-B binding mAbs against WT SARS-CoV-2. b, Neutralization potency of each mAb from each subject against WT SARS-CoV-2, B.1.1.7, P.1, B.1.617.1 and B.1.617.2. Each dot indicates one mAb. MAbs that neutralize VOCs are bolded. Data in a-c are representative of two independent experiments performed in duplicate. Data in a were analyzed using Mann-Whitney non-parametric test.

FIG. 8A-K. Proportion of SARS-CoV-2-specific B cells and characterization of RBD-reactive mAbs isolated from COVID-19 convalescent individuals. a-b, Uniform manifold approximation and projection (UMAP) of SARS-CoV-2 (a) spike RBD binding and (b) spike non-RBD binding B cells isolated from convalescent subjects that could be characterized into 3 groups (high, mid and low responder) based on their serological response against SARS-CoV-2 spike. c, Proportion of spike non-RBD- and spike RBD-specific binding B cells representing in each responder group. d-e, Number of somatic hypermutations in the IGHV in antibodies targeting (d) RBD and (e) non-RBD. f, Binding profile of RBD-reactive mAbs against single RBD mutants associated with different antibody classes, a combinatorial RBD mutant, and the RBDs of SARS-CoV-1 and MERS-CoV. Color gradients indicate relative binding percentage compared to spike WT g, Neutralization potency measured by plaque assay (complete inhibitory concentration; IC99) and focus reduction neutralization test (FRNT; half inhibitory concentration; IC50) of RBD-reactive mAbs to SARS-CoV-2 variants and sarbecoviruses. Binding breadth against full-length spike SARS-CoV-2 variants determined by ELISA is shown for (h) S728-1157, (i) S451-1140, and (j) S626-161. k, Heatmap represents area under curve (AUC) fold-change of broadly neutralizing RBD-reactive mAbs against ectodomain spike SARS-CoV-2 variants relative to WT-2P and the differences of AUC fold-change between spike BA.1-2P relative to spike BA.1-6P. The statistical analysis in d-e was determined using Kruskal-Wallis with Dunn's multiple comparison test. Data in f-g and h-j are representative of two independent experiments performed in duplicate. Genetic information for each antibody is in Table S2 (Example 2). The SARS-CoV-2 viruses used in neutralization assay are indicated in Table S4 (Example 2).

FIG. 9A-D: Mechanism of broad neutralization of S728-1157. (a) Epitope binning of broadly neutralizing RBD-reactive mAbs. Heatmap demonstrating the percentage of competition between each RBD-reactive mAb from previous studies with three broadly neutralizing mAbs, S728-1157, S451-1140 and S626-161. Data are representative of two independent experiments performed in triplicate. (b) Surface representation of the model derived from the cryoEM map of spike WT-6P-Mut7 in complex with IgG S728-1157. Although the inventors observe full mAb occupancy in the cryo-EM map, only one Fv is shown here. (c) Structural comparison of S728-1157 to other RBS-A antibodies such as CC12.1 (PDB ID: 6XC2), CC12.3 (PDB ID: 6XC4), B38 (PDB ID: 7BZ5), and C105 (PDB ID: 6XCN). The heavy chains are a darker shade, and the light chains are a lighter shade. Omicron BA.1 mutations near the epitope interface are shown as spheres. (d) CDR-H3 forms distinct interactions with SARS-CoV-2 RBD between S728-1157 and CC12.3. Sequence alignment of CDR-H3 of the two antibodies are shown in the middle with non-conserved residues.

FIG. 10A-G: Protective efficacy of broadly neutralizing antibodies against SARS-CoV-2 infection in hamster. Schematic illustrating the in vivo experiment schedule (a). Lung and nasal turbinate (NT) viral replication SARS-CoV-2 are shown for hamster treated therapeutically with (b-d) S728-1157 (n=3) (e) S451-1140 (n=3) and (f-g) S626-161 (n=4) at day 4 post-challenge with SARS-CoV-2 compared with control mAb, anti-Ebola surface glycoprotein (KZ52) antibody. Dashed horizontal lines represent the limit of detection (LOD) of the experiment. P-values in (b-g) were calculated using Unpaired t-test. The infected SARS-CoV-2 viruses are detailed in Table S4 (Example 2).

FIG. 11A-K: Convalescent serum antibody competition with broadly neutralizing RBD-reactive mAbs and comparison of serum antibody response against spike 6P-versus 2P-stabilized. Schematic diagram for experimental procedure of serum competitive ELISA (a). Half-maximal inhibitory concentration (EC50) of polyclonal antibody serum from convalescent individuals that could compete with broadly neutralizing mAbs (competitor mAb): S728-1157 (b), S451-1140 (c) and S626-161 (d), therapeutic neutralizing mAbs LY-CoV555 (e), REGN-10933 (f), non-neutralizing mAb CR3022 (g) and well-defined class 1 mAb CC12.3 (h). The reciprocal serum dilutions in b-h are showed as Log 1P of the IC50 of serum dilution that can achieve 50% competition with the competitor mAb of interest. The statistical analysis in b-h was determined using Kruskal-Wallis with Dunn's multiple comparison test. Representative three conformations of pre-fusion spike trimer antigen observed in the previous structural characterization of SARS-CoV-2 stabilized by 2P and 6P31,47 (i). Endpoint titer of convalescent sera against SARS-CoV-2 spike wildtype (WT) (j) and Omicron BA.1 (k) in two versions of spike substituted by 2P and 6P. Data in b-h and j-k are representative of two independent experiments performed in duplicate. Wilcoxon matched-pairs signed rank test was used to compare the anti-spike antibody titer against 2P and 6P in j-k. Fold change indicated in j-k is defined as the mean fold change.

FIG. 12: Amino acid and nucleotide sequences of complementarity-determining region (CDR) of heavy chain and light chain of the three bnAbs. Contacting residues within CDR of S728-1157 and SARS-CoV-2 are highlighted as light grey. Genetic information for each antibody is in Table S2 (Example 2). The sequences in the figure correspond to SEQ ID NO: 1883 (S728-1157 heavy chain amino acid sequence), SEQ ID NO:1884 (S728-1157 heavy chain nucleotide sequence), SEQ ID NO:1885 (S728-1157 light chain amino acid sequence), SEQ ID NO:1886 (S728-1157 light chain nucleotide sequence), SEQ ID NO: 1887 (S451-1140 heavy chain amino acid sequence), SEQ ID NO:1888 (S451-1140 heavy chain nucleotide sequence), SEQ ID NO: 1889 (S451-1140 light chain amino acid sequence), SEQ ID NO: 1890 (S451-1140 light chain nucleotide sequence), SEQ ID NO: 1891 (S626-161 heavy chain amino acid sequence), SEQ ID NO: 1892 (S626-161 heavy chain nucleotide sequence), SEQ ID NO: 1893 (S626-161 light chain amino acid sequence), and SEQ ID NO: 1894 (S626-161 light chain nucleotide sequence).

FIG. 13A-D: Broadly neutralizing RBD-reactive mAbs activity against SARS-CoV-2 and emerging variants. a, Structural models for the full-length spike protein variants and amino acid substitutions that encoded in B.1.1.7 Alpha, B.1.351 Beta, P.1 Gamma, B.1.617.2 Delta and Omicron, BA.1, BA.2 and BA.4. The structural models in a are modified from PDB ID: 6XM4. b, The table illustrating the binding rate and equilibrium constants (kₒₙ, k_{off}, and affinity binding K_{D}) measured by BLI of S728-1157, S451-1140 and S626-161 IgG in response to the panel of SARS-CoV-2 VOCs (either former or current VOCs). c, The binding rate comparison of Fabs of S728-1157, S451-1140 and S626-161 in responding to spike WT-6P and 2P constructs. The binding traces of IgG and Fab analyzed by BLI were represented by the 1:2 and 1:1 interaction model, respectively. d, The fold-change of binding rate (Kₒₙ, K_{off}) and binding affinity (K_{D}) between spike WT-6P and spike WT-2P bound by broadly neutralizing RBD-reactive mAbs, whole IgG form and Fab. Data in c-d are representative of two independent experiments, the data from experiments that have the best fit (R²>0.90) are selected for analysis.

FIG. 14A-F: Biolayer interferometry analysis demonstrates binding affinity curves of three broadly neutralizing mAbs competing with each other in response to biotinylated spike wildtype (WT)-6P (left panel) and spike BA.1 Omicron-6P (right panel). a-b, S626-161 was firstly bound, followed by S728-1157 mAb as competing mAb. c-d, S451-1140 was firstly bound and competed with S728-1157 and e-f, S626-161. The response curve was normalized in relation to its starting response value.

FIG. 15A-E. Structural analysis of S728-1157 binding to SARS-CoV-2 spike. (a) Three-dimensional (3D) reconstruction of Omicron BA.1-6P in complex with IgG S728-1157 shows binding by negative stain electron microscopy. The binding mode is the same as binding to spike WT-6P-Mut7 shown in FIG. 2b. (b) CDR-H1 of S728-1157 forms similar interactions with SARS-CoV-2 RBD compared to another IGHV3-53 antibody CC12.3 (PDB ID: 6XC4). (c) CDR-H2 of S728-1157 forms similar interactions with the RBD compared to CC12.3 (PDB ID: 6XC4). (d) For spike WT-6P-Mut7 in complex with S728-1157, residues Y505 and V_{L} Q31, and E484 and V_{L} Y99 are predicted to make hydrogen bonds. Hydrophobic residues Y486 and Y489 are shown as well. Since S728-1157 binds spike Omicron BA.1-6P in the same way as to spike WT-6P-Mut7, it may accommodate the E484A and Y505H mutations in Omicron. (e) Local resolution estimates of the cryo-EM map (upper panel) and local refinement on the RBD-Fv after symmetry expansion using RELION (lower panel).

### DETAILED DESCRIPTION

Several severe acute respiratory syndrome coronavirus 2 (SARS-CoV-2) variants have arisen that exhibit increased viral transmissibility and partial evasion of immunity induced by natural infection and vaccination. To address the specific antibody targets that were affected by recent viral variants, the inventors generated monoclonal antibodies (mAbs) from 10 convalescent donors that bound three distinct domains of the SARS-CoV-2 spike. Viral variants harboring mutations at K417, E484 and N501 could escape most of the highly potent antibodies against the receptor binding domain (RBD). Despite this, they identified neutralizing mAbs against three distinct regions of the spike protein that neutralize SARS-CoV-2 and the variants of concern, including B.1.1.7 (alpha), P.1 (gamma) and B.1.617.2 (delta). Notably, antibodies targeting distinct epitopes could neutralize discrete variants, suggesting different variants may have evolved to disrupt the binding of particular neutralizing antibody classes. These results underscore that humans exposed to first pandemic wave of prototype SARS-CoV-2 do possess neutralizing antibodies against current variants and that it is critical to induce antibodies targeting multiple distinct epitopes of the spike that can neutralize emerging variants of concern.

### I. Antibodies

The disclosure relates to antibodies, antigen binding fragments thereof, or polypeptides capable of specifically binding to a SARS-CoV-2 spike(S) protein, NP protein, or ORF8. Also described are antibodies, or fragments thereof, that specifically bind to a receptor binding domain (RBD) of a SARS-CoV-2 spike protein.

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4:302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (2). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

### A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. Bispecific antibodies can be biparatopic, wherein a bispecific antibody may specifically recognize a different epitope from the same antigen. Bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

The antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, included are bispecific, trispecific, tetraspecific, and other multispecific antibodies or antigen-binding fragments thereof that are directed to epitopes and to other targets, such as Fc receptors on effector cells.

Multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-L1, CDR-L2, and CDR-L3. The L3 (CDR-L3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as CDR-H1, CDR-H2 and CDR-H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include:
1) Computational predictions of the tertiary structure of the antibody/epitope binding interactions based on the chemical nature of the amino acid sequence of the antibody variable region and composition of the epitope.
2) Hydrogen-deuterium exchange and mass spectroscopy
3) Polypeptide fragmentation and peptide mapping approaches in which one generates multiple overlapping peptide fragments from the full length of the polypeptide and evaluates the binding affinity of these peptides for the epitope.
4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

Affinity matured antibodies may be enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24:8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

Portions of the heavy and/or light chain may be identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

Minimizing the antibody polypeptide sequence from the non-human species may optimize chimeric antibody function and reduce immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### B. Functional Antibody Fragments and Antigen-Binding Fragments

### 1. Antigen-Binding Fragments

The disclosure provides for antibody fragments, such as antibody fragments that bind to a SARS-CoV-2 spike protein. The term functional antibody fragment includes antigen-binding fragments of an antibody that retain the ability to specifically bind to an antigen. These fragments are constituted of various arrangements of the variable region heavy chain (VH) and/or light chain (VL); and may include constant region heavy chain 1 (CH1) and light chain (CL). They may also lack the Fc region constituted of heavy chain 2 (CH2) and 3 (CH3) domains. Antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment (also “Fab”) means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv) 2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv) 2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

### 2. Fragment Antigen Binding Region, Fab

Fab polypeptides of the disclosure include the Fab antigen binding fragment of an antibody. Unless specifically stated otherwise, the term “Fab” relates to a polypeptide excluding the Fc portion of the antibody. The Fab may be conjugated to a polypeptide comprising other components, such as further antigen binding domains, costimulatory domains, linkers, peptide spacers, transmembrane domains, endodomains, and accessory proteins. Fab polypeptides can be generated using conventional techniques known in the art and are well-described in the literature.

### 3. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

C. Polypeptides with antibody CDRs & Scaffolding Domains that Display the CDRs

Antigen-binding peptide scaffolds, such as complementarity-determining regions (CDRs), may be used to generate protein-binding molecules. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### D. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful may have an affinity constant (Ka) of about, at least about, or at most about 10⁶, 10⁷, 10⁸, 10⁹, or 10¹⁰ M or any range derivable therein. Similarly, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (K_{D}) is ≤10⁻⁸ M. The antibody specifically binds antigen with “high affinity” when the K_{D} is ≤5×10⁻⁹ M, and with “very high affinity” when the KD is ≤5×10⁻¹⁰ M.

### 2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

### 3. Modification of Antibody Antigen-Binding Domains

It is understood that the antibodies of the present invention may be modified, such that they are substantially identical to the antibody polypeptide sequences, or fragments thereof, and still bind the epitopes of the present invention. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present invention, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

### E. Chemical Modification of Antibodies

Also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). Antibody protein variants may comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. One or more new N-linked glycosylation sites may be created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

The polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the invention to obtain PEGylated derivatives of antibodies. See, e.g., EP 0 154 316 and EP 0 401 384. The antibody can be conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

### 1. Conjugation

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. Polypeptides may be chemically modified by conjugating or fusing the polypeptide to serum protein, such as human serum albumin, to increase half-life of the resulting molecule. See, e.g., EP 0322094 and EP 0 486 525. The polypeptides may be conjugated to a diagnostic agent and used diagnostically, for example, to monitor the development or progression of a disease and determine the efficacy of a given treatment regimen. The polypeptides may also be conjugated to a therapeutic agent to provide a therapy in combination with the therapeutic effect of the polypeptide. Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

Disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

a. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

Also contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. An antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

ADC can include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. Oligomers may comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

b. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). Also contemplated are derivatization of immunoglobulins by selectively introducing sulfhydryl groups in the Fc region of an immunoglobulin, using reaction conditions that do not alter the antibody combining site. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

### II. Antibody Production

### A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. Polypeptides, peptides, and proteins and immunogenic fragments thereof for use in methods and compositions of the disclosure can also be synthesized in solution or on a solid support in accordance with conventional techniques. See, for example, Stewart and Young, (1984); Tarn et al, (1983); Merrifield, (1986); and Barany and Merrifield (1979), each incorporated herein by reference.

Briefly, a polyclonal antibody is prepared by immunizing an animal with an antigen or a portion thereof and collecting antisera from that immunized animal. The antigen may be altered compared to an antigen sequence found in nature. A variant or altered antigenic peptide or polypeptide may be employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988).

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after two to three weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m2) (Johnson/Mead, NJ), cytokines such as B-interferon, IL-2, or IL-12, or genes encoding proteins involved in immune helper functions, such as B-7.A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

### B. Fully Human Antibody Production

Methods are available for making fully human antibodies. Using fully human antibodies can minimize the immunogenic and allergic responses that may be caused by administering non-human monoclonal antibodies to humans as therapeutic agents. Human antibodies may be produced in a non-human transgenic animal, e.g., a transgenic mouse capable of producing multiple isotypes of human antibodies to protein (e.g., IgG, IgA, and/or IgE) by undergoing V-D-J recombination and isotype switching. This may apply to antibodies, antibody fragments, and pharmaceutical compositions thereof, but also non-human transgenic animals, B-cells, host cells, and hybridomas that produce monoclonal antibodies. Applications of human antibodies include, but are not limited to, detect a cell expressing an anticipated protein, either in vivo or in vitro, pharmaceutical preparations containing the antibodies of the present invention, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et al., Year in Immunol. 7:33 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (μ and γ) and κ light chain immunoglobulin sequences, together with targeted mutations that inactivate the endogenous μ and κ chain loci (Lonberg et al., Nature 368:856-859 (1994)). Accordingly, the mice exhibit reduced expression of mouse IgM or κ chains and in response to immunization, the introduced human heavy and light chain transgenes undergo class switching and somatic mutation to generate high affinity human IgG K monoclonal antibodies (Lonberg et al., supra; Lonberg and Huszar, Intern. Ref. Immunol. 13:65-93 (1995); Harding and Lonberg, Ann. N.Y. Acad. Sci. 764:536-546 (1995)). The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref. Immunol. 13:65-93 (1995); Harding and Lonberg, Ann. N. Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)). One such technique is described in International Patent Application Publication No. WO 99/10494 (herein incorporated by reference), which describes the isolation of high affinity and functional agonistic antibodies for MPL- and msk-receptors using such an approach.

### C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (VL and VH). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different VL- and VH-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, VL and VH, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be generated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003) also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the invention are proteins that are structurally similar to an antibody displaying a desired biological activity, such as the ability to bind a protein, but have one or more peptide linkages optionally replaced by a linkage selected from: —CH2NH—, —CH2S—, —CH2-CH2-, —CH═CH-(cis and trans), —COCH2-, —CH(OH)CH2-, and —CH2SO— by methods well known in the art. Systematic substitution of one or more amino acids of a consensus sequence with a D-amino acid of the same type (e.g., D-lysine in place of L-lysine) may be used to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

### III. Obtaining Encoded Antibodies

Also provided are nucleic acid molecule encoding antibody polypeptides (e.g., heavy or light chain, variable domain only, or full-length). These may be generated by methods known in the art, e.g., isolated from B cells of mice that have been immunized and isolated, phage display, expressed in any suitable recombinant expression system and allowed to assemble to form antibody molecules.

### A. Expression

The nucleic acid molecules may be used to express large quantities of recombinant antibodies or to produce chimeric antibodies, single chain antibodies, immunoadhesins, diabodies, mutated antibodies, and other antibody derivatives. If the nucleic acid molecules are derived from a non-human, non-transgenic animal, the nucleic acid molecules may be used for antibody humanization.

### 1. Vectors

Also contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. Expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the antibodies, or antigen-binding fragments thereof, DNAs encoding partial or full-length light and heavy chains are inserted into expression vectors such that the gene area is operatively linked to transcriptional and translational control sequences. A vector that encodes a functionally complete human CH or CL immunoglobulin sequence with appropriate restriction sites engineered so that any VH or VL sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 2. Expression Systems

Numerous expression systems exist that comprise at least a part or all of the expression vectors discussed above. Prokaryote- and/or eukaryote-based systems can be employed for use to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### 3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 4. Host Cells

Also contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. The antibody expression construct can be placed under control of a promoter that is linked to T-cell activation, such as one that is controlled by NFAT-1 or NF-κB, both of which are transcription factors that can be activated upon T-cell activation. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### IV. Viruses

The disclosure relates to treatment, analysis, or use of a virus. Disclosed are methods for treatment or prevention of a viral infection. Also disclosed are compositions comprising one or more anti-viral agents. Also disclosed are methods for diagnosis of a viral infection. Also disclosed are methods for detection of a virus in a sample.

### A. Coronaviruses

The virus may be from the family Coronaviridae. Coronaviridae is a family of enveloped, positive-sense, single-stranded RNA viruses. Coronavirus is the common name for Coronaviridae and Orthocoronavirinae (also referred to as Coronavirinae). The family Coronaviridae is organized in 2 sub-families, 5 genera, 23 sub-genera and approximately 40 species. They are enveloped viruses having a positive-sense single-stranded RNA genome and a nucleocapsid having helical symmetry. The genome size of coronaviruses ranges from approximately 26-32 kilobases.

The present disclosure encompasses treatment or prevention of infection of any virus in the Coronaviridae family. The disclosure may encompass treatment or prevention of infection of any virus in the subfamily Coronavirinae and including the four genera, Alpha-, Beta-, Gamma-, and Deltacoronavirus. The disclosure may include treatment or prevention of infection of any virus in the genus of Betacoronavirus, including the subgenus Sarbecovirus and including the species of severe acute respiratory syndrome-related coronavirus. The disclosure may encompass treatment or prevention of infection of any virus in the species of severe acute respiratory syndrome-related coronavirus, including the strains severe acute respiratory syndrome coronavirus (SARS-CoV) and severe acute respiratory syndrome coronavirus 2 (SARS-CoV-2, the virus that causes COVID-19). The disclosure encompasses treatment or prevention of infection any isolate, strain, type (including Type A, Type B and Type C; Forster et al., 2020, PNAS, available on the World Wide Web at doi.org/10.1073/pnas.2004999117), cluster, or sub-cluster of the species of severe acute respiratory syndrome-related coronavirus, including at least SARS-CoV-2. The virus may have a genome length between 29000 to 30000, between 29100 and 29900, between 29200 and 29900, between 29300 and 29900, between 29400 and 29900, between 29500 and 29900, between 29600 and 29900, between 29700 and 29900, between 29800 and 29900, or between 29780 and 29900 base pairs in length.

Examples of specific SARS-CoV-2 viruses include the following listed in the NCBI GenBank® Database, and these GenBank® Accession sequences are incorporated by reference herein in their entirety: (a) LC534419 and LC534418 and LC528233 and LC529905 (examples of different strains from Japan); (b) MT281577 and MT226610 and NC_045512 and MN996531 and MN908947 (examples of different strains from China); (c) MT281530 (Iran); (d) MT126808 (Brazil); (e) MT020781 (Finland); (f) MT093571 (Sweden); (g) MT263074 (Peru); (h) MT292582 and MT292581 and MT292580 and MT292579 (examples of different strains from Spain); (i) examples from the United States, such as MT276331 (TX); MT276330 (FL); MT276328 (OR) MT276327 (GA); MT276325 (WA); MT276324 (CA); MT276323 (RI); MT188341 (MN); and (j) MT276598 (Israel). The disclosure may include treatment or prevention of infection of any of these or similar viruses, including viruses whose genome has at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. The disclosure includes treatment or prevention of infection of any of these or similar viruses, including viruses whose genome has its entire sequence that is greater than 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. As one specific example, the present disclosure includes methods of treatment or prevention of infection of a virus having a genome sequence of SEQ ID NO:110 (represented by GenBank® Accession No. NC_045512; origin Wuhan, China) and any virus having a genome sequence with at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to SEQ ID NO: 110.

SARS-CoV-2 proteins are described in detail in, for example, Yoshimoto F. K. (2020). The protein journal, 39 (3), 198-216, incorporated herein by reference in its entirety.

### V. Antibodies, Antigen Binding Fragments, and Polypeptides

As used herein, a “protein” or “polypeptide” refers to a molecule comprising at least five amino acid residues. As used herein, the term “wild-type” refers to the endogenous version of a molecule that occurs naturally in an organism. Wild-type versions of a protein or polypeptide are employed, however, a modified protein or polypeptide may be employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. A modified/variant protein or polypeptide may have at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity. The term polypeptide also includes and antibody fragment described herein as well as antibody domains, such as HCDR1, HCDR2, HCDR3, LCDR1, LCDR2, LCDR3, HFRW1, HFRW2, HFRW3, HFRW4, LFRW1, LFRW2, LFRW3, LFRW4, VH, VL, CH, or CL.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. Also described are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

The size of an antibody, antigen binding fragment, protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The antibody, antigen binding fragment, polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NO: 1-3028.

The antibody, antigen binding fragment, protein, or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOS: 1-3028.

The antibody, antigen binding fragment, or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs: 1-2706.

The antibody, antigen binding fragment, protein, or polypeptide may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOS: 1-3028 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS: 1-3028.

Also provided is a nucleic acid molecule, antibody, antigen binding fragment, protein, or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS: 1-3028 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS: 1-3028.

The amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of the heavy chain, light chain, VH, VL, HCDR1, HCDR2, HCDR3, LCDR1, LCDR2, LCDR3, HFRW1, HFRW2, HFRW3, HFRW4, LFRW1, LFRW2, LFRW3, or LFRW4 identified in Table 1 is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

A polypeptide (e.g., antibody, antibody fragment, Fab, etc.) of the disclosure comprises a CDR that is at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any range derivable therein) in sequence to a HCDR or LCDR identified in Table 1. A polypeptide may comprise 1, 2, and/or 3 CDRs from a heavy chain or light chain variable region identified in Table 1. The CDR may be one that has been determined by Kabat, IMGT, or Chothia. A polypeptide may have CDRs that have 1, 2, and/or 3 amino acid changes (e.g., addition of 1 or 2 amino acids, deletions of 1 or 2 amino acids, substitution) with respect to these 1, 2, or 3 CDRs. A polypeptide may comprise additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

From amino to carboxy terminus the CDRs are CDR1, CDR2, and CDR3. A polypeptide may have CDRs that have 1, 2, and/or 3 amino acid changes (e.g., addition of 1 or 2 amino acids, deletions of 1 or 2 amino acids, substitution) with respect to CDR1, CDR2, or CDR3. The CDRs identified in Table 1 may further comprise 1, 2, 3, 4, 5, or 6 additional amino acids at the amino or carboxy terminus of the CDR, The additional amino acids may be from the heavy and/or light chain framework regions of SEQ ID NOS: 44-76, that are shown as immediately adjacent to the CDRs. Accordingly, also described are polypeptides comprising an HCDR1 (i.e., CDR-H1), HCDR2 (i.e., CDR-H2), HCDR3 (i.e., CDR-H3), LCDR1 (i.e., CDR-L1), LCDR2 (i.e., CDR-L2), and/or LCDR3 (i.e., CDR-L3) with at least or at most or exactly 1, 2, 3, 4, 5, 6 or 7 amino acids at the amino end of the CDR or at the carboxy end of the CDR, wherein the additional amino acids are the 1, 2, 3, 4, 5, 6, or 7 amino acids that are shown as immediately adjacent to the CDRs in a variable region of Table 1. Also included are antibodies comprising one or more CDRs, wherein the CDR is a fragment of a CDR identified in Table 1 and wherein the fragment lacks 1, 2, 3, 4, or 5 amino acids from the amino or carboxy end of the CDR. The CDR may lack one, 2, 3, 4, 5, 6, or 7 amino acids from the carboxy end and may further comprise 1, 2, 3, 4, 5, 6, 7, or 8 amino acids from the framework region of the amino end of the CDR. The CDR may lack one, 2, 3, 4, 5, 6, or 7 amino acids from the amino end and may further comprise 1, 2, 3, 4, 5, 6, 7, or 8 amino acids from the framework region of the carboxy end of the CDR. An antibody may be alternatively or additionally humanized in regions outside the CDR(s) and/or variable region(s). A polypeptide may comprise, additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

A polypeptide or protein may comprise 1, 2, 3, 4, 5, or 6 CDRs from either or both of the light and heavy variable regions of an antibody clone identified in Table, and 1, 2, 3, 4, 5, or 6 CDRs may have 1, 2, and/or 3 amino acid changes with respect to these CDRs. Parts or all of the antibody sequence outside the variable region may have been humanized. A protein may comprise one or more polypeptides. A protein may contain one or two polypeptides similar to a heavy chain polypeptide and/or 1 or 2 polypeptides similar to a light chain polypeptide.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### VI. Sequences

Polypeptide, antibody, and antigen binding fragments are shown below in the following tables. In the table below, HC refers to heavy chain (including the heavy chain variable and constant regions), and LC refers to light chain (including the light chain variable and constant regions). HCDR1, HCDR2, and HCDR3 are the heavy chain complementarity-determining regions, and LCDR1, LCDR2, and LCDR3 are the light chain complementarity-determining regions. HFR1, HFR2, HFR3, and HFR4 are the framework regions of the heavy chain variable region, and LFR1, LFR2, LFR3, and LFR4 are the framework regions of the light chain variable region. HC variable refers to the heavy chain variable region, and LC variable refers to the light chain variable region.

TABLE 1
Antibody and antigen binding embodiments

| | | | SEQ |
|---|---|---|---|
| | Descrip- | | ID |
| Clone | tion | Sequence | NO: |
| S20-15 | HC | QVQLQESGPGLVRPSETLSLTCTVS | 1 |
| | | GGSISSHYWSWIRQPPGKGLEWIGY | |
| | | IYYSGSTNYNPSLKSRVTISVDTSK | |
| | | NQFSLKLISVTAADTAVYYCARAGG | |
| | | VFGVVLDFDHWGRGTLVTVSSASTK | |
| | | GPSVFPLAPSSKSTSGGTAALGCLV | |
| | | KDYFPEPVTVSWNSGALTSGVHTFP | |
| | | AVLQSSG | |
| | HC | QVQLQESGPGLVRPSETLSLTCTVS | 2 |
| | variable | GGSISSHYWSWIRQPPGKGLEWIGY | |
| | | IYYSGSTNYNPSLKSRVTISVDTSK | |
| | | NQFSLKLISVTAADTAVYYCARAGG | |
| | | VFGVVLDFDHWGRGTLVTVSS | |
| | HCDR1 | SHYWS | 3 |
| | HCDR2 | YIYYSGSTNYNPSLKS | 4 |
| | HCDR3 | AGGVFGVVLDFDH | 5 |
| | HFR1 | QVQLQESGPGLVRPSETLSLTCTVS | 6 |
| | | GGSIS | |
| | HFR2 | WIRQPPGKGLEWIG | 7 |
| | HFR3 | RVTISVDTSKNQFSLKLISVTAADT | 8 |
| | | AVYYCAR | |
| | HFR4 | WGRGTLVTVSS | 9 |
| | LC | SYVLTQPPSVSVAPGQTARITCGGN | 10 |
| | | NIGSKSVHWYQQKPGQAPVLVVYDD | |
| | | SDRPSGIPERFSGSNSGNTATLTIS | |
| | | RVEAGDEADYYCQVWDSSSEHYVFG | |
| | | TGTKVTVLGQPKANPTVTLFPPSSE | |
| | | ELQANKATLVCLISDFYPGAVTVAW | |
| | | KADGSPVKAGVETTKPSKQSNNKYA | |
| | | ASS | |
| | LC | SYVLTQPPSVSVAPGQTARITCGGN | 11 |
| | variable | NIGSKSVHWYQQKPGQAPVLVVYDD | |
| | | SDRPSGIPERFSGSNSGNTATLTIS | |
| | | RVEAGDEADYYCQVWDSSSEHYVFG | |
| | | TGTKVTVL | |
| | LCDR1 | GGNNIGSKSVH | 12 |
| | LCDR2 | DDSDRPS | 13 |
| | LCDR3 | QVWDSSSEHYV | 14 |
| | LFR1 | SYVLTQPPSVSVAPGQTARITC | 15 |
| | LFR2 | WYQQKPGQAPVLVVY | 16 |
| | LFR3 | GIPERFSGSNSGNTATLTISRVEAG | 17 |
| | | DEADYYC | |
| | LFR4 | FGTGTKVTVL | 18 |
| S20-22 | HC | QVQLQESGPGLVKPSETLSLTCTVS | 19 |
| | | GGSISSFYWGWIRQPAGKGLEWIGR | |
| | | FHTSGSTNYNPSFKSRVTMSVDTSK | |
| | | NQFSLKLTSVTAADTAVYYCASGRG | |
| | | SSWYVGWFFDLWGRGTLVTVSSAST | |
| | | KGPSVFPLAPSSKSTSGGTAALGCL | |
| | | VKDYFPEPVTVSWNSGALTSGVHTF | |
| | | PAVLQSSG | |
| | HC | QVQLQESGPGLVKPSETLSLTCTVS | 20 |
| | variable | GGSISSFYWGWIRQPAGKGLEWIGR | |
| | | FHTSGSTNYNPSFKSRVTMSVDTSK | |
| | | NQFSLKLTSVTAADTAVYYCASGRG | |
| | | SSWYVGWFFDLWGRGTLVTVSS | |
| | HCDR1 | SFYWG | 21 |
| | HCDR2 | RFHTSGSTNYNPSFKS | 22 |
| | HCDR3 | GRGSSWYVGWFFDL | 23 |
| | HFR1 | QVQLQESGPGLVKPSETLSLTCTVS | 24 |
| | | GGSIS | |
| | HFR2 | WIRQPAGKGLEWIG | 25 |
| | HFR3 | RVTMSVDTSKNQFSLKLTSVTAADT | 26 |
| | | AVYYCAS | |
| | HFR4 | WGRGTLVTVSS | 9 |
| | LC | DIVMTQSPDSLAVSLGERATINCKS | 27 |
| | | SQTVLYSSNNKNYLAWYQQKPGQPP | |
| | | KLLIYWASTRESGVPDRFSGSGSGT | |
| | | DFTLTISSLQAGDVAVYYCQQYYNT | |
| | | PDTFGGGTKVEINRTVAAPSVFIFP | |
| | | PSDEQLKSGTASVVCLLNNFYPREA | |
| | | KVQWKVDN | |
| | LC | DIVMTQSPDSLAVSLGERATINCKS | 28 |
| | variable | SQTVLYSSNNKNYLAWYQQKPGQPP | |
| | | KLLIYWASTRESGVPDRFSGSGSGT | |
| | | DFTLTISSLQAGDVAVYYCQQYYNT | |
| | | PDTFGGGTKVEI | |
| | LCDR1 | KSSQTVLYSSNNKNYLA | 29 |
| | LCDR2 | WASTRES | 30 |
| | LCDR3 | QQYYNTPDT | 31 |
| | LFR1 | DIVMTQSPDSLAVSLGERATINC | 32 |
| | LFR2 | WYQQKPGQPPKLLIY | 33 |
| | LFR3 | GVPDRFSGSGSGTDFTLTISSLQAG | 34 |
| | | DVAVYYC | |
| | LFR4 | FGGGTKVEI | 35 |
| S20-31 | HC | QVQLIQSGAEVKKPGASVKVSCTAS | 36 |
| | | GYSLNELPIQWVRQAPGKGLEWMGE | |
| | | FDPEDGETIYAEKFQGRVTLTEETS | |
| | | TNTAYMELSSLKSEDTAAYFCSTGS | |
| | | TIGVVIYAFAIWGQGTMVTVSSAST | |
| | | KGPSVFPLAPCSRSTSESTAALGCL | |
| | | VKDYFPEPVTVSWNSGALTSGVHTF | |
| | | PAVLQSSG | |
| | HC | QVQLIQSGAEVKKPGASVKVSCTAS | 37 |
| | variable | GYSLNELPIQWVRQAPGKGLEWMGE | |
| | | FDPEDGETIYAEKFQGRVTLTEETS | |
| | | TNTAYMELSSLKSEDTAAYFCSTGS | |
| | | TIGVVIYAFAIWGQGTMVTVSS | |
| | HCDR1 | ELPIQ | 38 |
| | HCDR2 | EFDPEDGETIYAEKFQG | 39 |
| | HCDR3 | GSTIGVVIYAFAI | 40 |
| | HFR1 | QVQLIQSGAEVKKPGASVKVSCTAS | 41 |
| | | GYSLN | |
| | HFR2 | WVRQAPGKGLEWMG | 42 |
| | HFR3 | RVTLTEETSTNTAYMELSSLKSEDT | 43 |
| | | AAYFCST | |
| | HFR4 | WGQGTMVTVSS | 44 |
| | LC | EIVLTQSPGTLSLSPGERATLSCRA | 45 |
| | | SQDITNNFLAWYQQKAGQAPKLFIY | |
| | | GASRRAPGIPHRFSGSGSGTDFTLT | |
| | | ISSLEPEDFAVYYCQQYGPSPTFGQ | |
| | | GTKVEIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | EIVLTQSPGTLSLSPGERATLSCRA | 46 |
| | variable | SQDITNNFLAWYQQKAGQAPKLFIY | |
| | | GASRRAPGIPHRFSGSGSGTDFTLT | |
| | | ISSLEPEDFAVYYCQQYGPSPTFGQ | |
| | | GTKVEIK | |
| | LCDR1 | RASQDITNNFLA | 47 |
| | LCDR2 | GASRRAP | 48 |
| | LCDR3 | QQYGPSPT | 49 |
| | LFR1 | EIVLTQSPGTLSLSPGERATLSC | 50 |
| | LFR2 | WYQQKAGQAPKLFIY | 51 |
| | LFR3 | GIPHRFSGSGSGTDFTLTISSLEPE | 52 |
| | | DFAVYYC | |
| | LFR4 | FGQGTKVEIK | 53 |
| S20-40 | HC | QVQLQESGPGLVKPSETLSLTCTVS | 54 |
| | | GGSISSYYWSWIRQPAGKGLEWIGR | |
| | | IYTSGSTNYNPSLKSRVTMSVDTSK | |
| | | NQFSLKLSSVTAADTAVYYCARGGS | |
| | | GWRFDYWGQGTLVTVSSGSASAPTL | |
| | | FPLVSCENSPSDTSSV | |
| | HC | QVQLQESGPGLVKPSETLSLTCTVS | 55 |
| | variable | GGSISSYYWSWIRQPAGKGLEWIGR | |
| | | IYTSGSTNYNPSLKSRVTMSVDTSK | |
| | | NQFSLKLSSVTAADTAVYYCARGGS | |
| | | GWRFDYWGQGTLVTVSS | |
| | HCDR1 | SYYWS | 56 |
| | HCDR2 | RIYTSGSTNYNPSLKS | 57 |
| | HCDR3 | GGSGWRFDY | 58 |
| | HFR1 | QVQLQESGPGLVKPSETLSLTCTVS | 24 |
| | | GGSIS | |
| | HFR2 | WIRQPAGKGLEWIG | 25 |
| | HFR3 | RVTMSVDTSKNQFSLKLSSVTAADT | 59 |
| | | AVYYCAR | |
| | HFR4 | WGQGTLVTVSS | 60 |
| | LC | QSALTQPASVSGSPGQSITISCTGT | 61 |
| | | SSDVGGYNYVSWYQQHPGKAPKLMI | |
| | | YDVSNRPSGVSNRFSGSKSGNTASL | |
| | | TISGLQAEDEADYYCSSYTSSSTLG | |
| | | VFGGGTKLTVLGQPKAAPSVTLFPP | |
| | | SSEELQANKATLVCLISDFYPGAVT | |
| | | VAWKADSSPVKAGVETTTPSKQSNN | |
| | | KYAASS | |
| | LC | QSALTQPASVSGSPGQSITISCTGT | 62 |
| | variable | SSDVGGYNYVSWYQQHPGKAPKLMI | |
| | | YDVSNRPSGVSNRFSGSKSGNTASL | |
| | | TISGLQAEDEADYYCSSYTSSSTLG | |
| | | VFGGGTKLTVL | |
| | LCDR1 | TGTSSDVGGYNYVS | 63 |
| | LCDR2 | DVSNRPS | 64 |
| | LCDR3 | SSYTSSSTLGV | 65 |
| | LFR1 | QSALTQPASVSGSPGQSITISC | 66 |
| | LFR2 | WYQQHPGKAPKLMIY | 67 |
| | LFR3 | GVSNRFSGSKSGNTASLTISGLQAE | 68 |
| | | DEADYYC | |
| | LFR4 | FGGGTKLTVL | 69 |
| S20-58 | HC | QVQLQESGPGLVKPSQTLSLTCTVS | 70 |
| | | GGSINSGDYYWSWIRQPPGKGLEWI | |
| | | GYIYFSGSTYYNPSLKSRVTISLDR | |
| | | SKNQFSLKLSSVTAADTAVYYCARE | |
| | | ESMITLGGVIVDWGQGTLVTVSSAS | |
| | | TKGPSVFPLAPSSKSTSGGTAALGC | |
| | | LVKDYFPEPVTVSWNSGALTSGVHT | |
| | | FPAVLQSSG | |
| | HC | QVQLQESGPGLVKPSQTLSLTCTVS | 71 |
| | variable | GGSINSGDYYWSWIRQPPGKGLEWI | |
| | | GYIYFSGSTYYNPSLKSRVTISLDR | |
| | | SKNQFSLKLSSVTAADTAVYYCARE | |
| | | ESMITLGGVIVDWGQGTLVTVSS | |
| | HCDR1 | SGDYYWS | 72 |
| | HCDR2 | YIYFSGSTYYNPSLKS | 73 |
| | HCDR3 | EESMITLGGVIVD | 74 |
| | HFR1 | QVQLQESGPGLVKPSQTLSLTCTVS | 75 |
| | | GGSIN | |
| | HFR2 | WIRQPPGKGLEWIG | 7 |
| | HFR3 | RVTISLDRSKNQFSLKLSSVTAADT | 76 |
| | | AVYYCAR | |
| | HFR4 | WGQGTLVTVSS | 60 |
| | LC | DIVMTQTPLSSPVTLGQPASISCRS | 77 |
| | | SQSLVHSDGDTYLSWLQQRPGQPPR | |
| | | LLIYKISNRFSGVPDRFSGSGAGTD | |
| | | FTLKISRVEAEDVGVYYCMQATQFP | |
| | | LTFGGGTKVEIKRTVAAPSVFIFPP | |
| | | SDEQLKSGTASVVCLLNNFYPREAK | |
| | | VQWKVDNALQSGNSQESVTEQDSKD | |
| | | STYSLSSTLTLSKADYE | |
| | LC | DIVMTQTPLSSPVTLGQPASISCRS | 78 |
| | variable | SQSLVHSDGDTYLSWLQQRPGQPPR | |
| | | LLIYKISNRFSGVPDRFSGSGAGTD | |
| | | FTLKISRVEAEDVGVYYCMQATQFP | |
| | | LTFGGGTKVEIK | |
| | LCDR1 | RSSQSLVHSDGDTYLS | 79 |
| | LCDR2 | KISNRFS | 80 |
| | LCDR3 | MQATQFPLT | 81 |
| | LFR1 | DIVMTQTPLSSPVTLGQPASISC | 82 |
| | LFR2 | WLQQRPGQPPRLLIY | 83 |
| | LFR3 | GVPDRFSGSGAGTDFTLKISRVEAE | 84 |
| | | DVGVYYC | |
| | LFR4 | FGGGTKVEIK | 85 |
| S20-74 | HC | QVQLQESGPGLVKPSETLSLTCTVS | 86 |
| | | GGSISSHYWSWIRQPPGKGLEQIGY | |
| | | MYYSGSTNYNPSLKSRVIISVDTSK | |
| | | NQFSLKLSSVTAADTAVYYCAGRDQ | |
| | | LLYGADGFDIWGQGTMVTVSSASTK | |
| | | GPSVFPLAPSSKSTSGGTAALGCLV | |
| | | KDYFPEPVTVSWNSGALTSGVHTFP | |
| | | AVLQSSG | |
| | HC | QVQLQESGPGLVKPSETLSLTCTVS | 87 |
| | variable | GGSISSHYWSWIRQPPGKGLEQIGY | |
| | | MYYSGSTNYNPSLKSRVIISVDTSK | |
| | | NQFSLKLSSVTAADTAVYYCAGRDQ | |
| | | LLYGADGFDIWGQGTMVTVSS | |
| | HCDR1 | SHYWS | 3 |
| | HCDR2 | YMYYSGSTNYNPSLKS | 88 |
| | HCDR3 | RDQLLYGADGFDI | 89 |
| | HFR1 | QVQLQESGPGLVKPSETLSLTCTVS | 24 |
| | | GGSIS | |
| | HFR2 | WIRQPPGKGLEQIG | 90 |
| | HFR3 | RVIISVDTSKNQFSLKLSSVTAADT | 91 |
| | | AVYYCAG | |
| | HFR4 | WGQGTMVTVSS | 44 |
| | LC | QSALTQPPSASGSPGQSVTISCTGT | 92 |
| | | SSDVGGYNYVSWYQQHPGKAPKLMI | |
| | | YEVSKRPSGVPDRYSGSKSGNTASL | |
| | | TVSGLQAEDEADYYCSSYAGSSNHV | |
| | | IFGGGTKLTVLGQPKAAPSVTLFPP | |
| | | SSEELQANKATLVCLISDFYPGAVT | |
| | | VAWKADSSPVKAGVETTTPSKQSNN | |
| | | KYAASS | |
| | LC | QSALTQPPSASGSPGQSVTISCTGT | 93 |
| | variable | SSDVGGYNYVSWYQQHPGKAPKLMI | |
| | | YEVSKRPSGVPDRYSGSKSGNTASL | |
| | | TVSGLQAEDEADYYCSSYAGSSNHV | |
| | | IFGGGTKLTVL | |
| | LCDR1 | TGTSSDVGGYNYVS | 63 |
| | LCDR2 | EVSKRPS | 94 |
| | LCDR3 | SSYAGSSNHVI | 95 |
| | LFR1 | QSALTQPPSASGSPGQSVTISC | 96 |
| | LFR2 | WYQQHPGKAPKLMIY | 67 |
| | LFR3 | GVPDRYSGSKSGNTASLTVSGLQAE | 97 |
| | | DEADYYC | |
| | LFR4 | FGGGTKLTVL | 69 |
| S20-86 | HC | EVQLVESGGGLVQPGRSLRLSCAAS | 98 |
| | | GFTFGDYAMYWVRQPPGKGLEWVSG | |
| | | ISWNRGTIGYADSVKGRFTISRDNA | |
| | | KNSLYLQMNSLTPEDTALYYCAKDM | |
| | | LPASRFFYYMDVWGKGTTVIVSSAS | |
| | | TKGPSVFPLAPSSKSTSGGTAALGC | |
| | | LVKDYFPEPVTVSWNSGALTSGVHT | |
| | | FPAVLQSSG | |
| | HC | EVQLVESGGGLVQPGRSLRLSCAAS | 99 |
| | variable | GFTFGDYAMYWVRQPPGKGLEWVSG | |
| | | ISWNRGTIGYADSVKGRFTISRDNA | |
| | | KNSLYLQMNSLTPEDTALYYCAKDM | |
| | | LPASRFFYYMDVWGKGTTVIVSS | |
| | HCDR1 | DYAMY | 100 |
| | HCDR2 | GISWNRGTIGYADSVKG | 101 |
| | HCDR3 | DMLPASRFFYYMDV | 102 |
| | HFR1 | EVQLVESGGGLVQPGRSLRLSCAAS | 103 |
| | | GFTFG | |
| | HFR2 | WVRQPPGKGLEWVS | 104 |
| | HFR3 | RFTISRDNAKNSLYLQMNSLTPEDT | 105 |
| | | ALYYCAK | |
| | HFR4 | WGKGTTVIVSS | 106 |
| | LC | QSALTQPASVSGSPGQSITISCTGT | 107 |
| | | SSDVGGYNYVSWYQQHPGKAPKLMI | |
| | | YDVSNRPSGVSNRFSGSKSGNTASL | |
| | | TISGLQAEDEADYYCSSYTSSSTLG | |
| | | VFGTGTKVTVLGQPKANPTVTLFPP | |
| | | SSEELQANKATLVCLISDFYPGAVT | |
| | | VAWKADGSPVKAGVETTKPSKQSNN | |
| | | KYAASS | |
| | LC | QSALTQPASVSGSPGQSITISCTGT | 108 |
| | variable | SSDVGGYNYVSWYQQHPGKAPKLMI | |
| | | YDVSNRPSGVSNRFSGSKSGNTASL | |
| | | TISGLQAEDEADYYCSSYTSSSTLG | |
| | | VFGTGTKVTVL | |
| | LCDR1 | TGTSSDVGGYNYVS | 63 |
| | LCDR2 | DVSNRPS | 64 |
| | LCDR3 | SSYTSSSTLGV | 65 |
| | LFR1 | QSALTQPASVSGSPGQSITISC | 66 |
| | LFR2 | WYQQHPGKAPKLMIY | 67 |
| | LFR3 | GVSNRFSGSKSGNTASLTISGLQAE | 68 |
| | | DEADYYC | |
| | LFR4 | FGTGTKVTVL | 18 |
| S24-68 | HC | QVQLQESGPGLVKPSETLSLTCTVS | 109 |
| | | GGSITSYYWSWIRQPPGKGLEWIEY | |
| | | IHYSGSTNYNPSLKSRVTISVDTSK | |
| | | NQFSLKLSSVTAADTAVYYCARLLK | |
| | | YSRGGCYFDHWGQGTLVTVSSASTK | |
| | | GPSVFPLAPSSKSTSGGTAALGCLV | |
| | | KDYFPEPVTVSWNSGALTSGVHTFP | |
| | | AVLQSSG | |
| | HC | QVQLQESGPGLVKPSETLSLTCTVS | 110 |
| | variable | GGSITSYYWSWIRQPPGKGLEWIEY | |
| | | IHYSGSTNYNPSLKSRVTISVDTSK | |
| | | NQFSLKLSSVTAADTAVYYCARLLK | |
| | | YSRGGCYFDHWGQGTLVTVSS | |
| | HCDR1 | SYYWS | 56 |
| | HCDR2 | YIHYSGSTNYNPSLKS | 111 |
| | HCDR3 | LLKYSRGGCYFDH | 112 |
| | HFR1 | QVQLQESGPGLVKPSETLSLTCTVS | 113 |
| | | GGSIT | |
| | HFR2 | WIRQPPGKGLEWIE | 114 |
| | HFR3 | RVTISVDTSKNQFSLKLSSVTAADT | 115 |
| | | AVYYCAR | |
| | HFR4 | WGQGTLVTVSS | 60 |
| | LC | QSVLTQPPSASGTPGQRVTISCSGS | 116 |
| | | SSNIGGNPVNWYQQLPGTAPKLLIY | |
| | | SNNQRPSGVPDRFSGSKSGTSASLA | |
| | | ISGLQSEDEADYYCAAWDDSLKGPV | |
| | | FGGGTKLTVLGQPKAAPSVTLFPPS | |
| | | SEELQANKATLVCLISDFYPGAVTV | |
| | | AWKADSSPVKAGVETTTPSKQSNNK | |
| | | YAASSYLSLTPEQWKSH | |
| | LC | QSVLTQPPSASGTPGQRVTISCSGS | 117 |
| | variable | SSNIGGNPVNWYQQLPGTAPKLLIY | |
| | | SNNQRPSGVPDRFSGSKSGTSASLA | |
| | | ISGLQSEDEADYYCAAWDDSLKGPV | |
| | | FGGGTKLTVL | |
| | LCDR1 | SGSSSNIGGNPVN | 118 |
| | LCDR2 | SNNQRPS | 119 |
| | LCDR3 | AAWDDSLKGPV | 120 |
| | LFR1 | QSVLTQPPSASGTPGQRVTISC | 121 |
| | LFR2 | WYQQLPGTAPKLLIY | 122 |
| | LFR3 | GVPDRFSGSKSGTSASLAISGLQSE | 123 |
| | | DEADYYC | |
| | LFR4 | FGGGTKLTVL | 69 |
| S24-105 | HC | EVQLVESGGGLVQPGGSLRLSCAAS | 124 |
| | | GFTLSSYSMNWVRQAPGKGLEWVSY | |
| | | ISSSSSTIYYADSVKGRFTISKDNA | |
| | | KNSLYLQMNSLRAEDTAVYYCAVGR | |
| | | GYFVYWGQGTLVTVSSASTKGPSVF | |
| | | PLAPSSKSTSGGTAALGCLVKDYFP | |
| | | EPVTVSWNSGALTSGVHTFPAVLQS | |
| | | SG | |
| | HC | EVQLVESGGGLVQPGGSLRLSCAAS | 125 |
| | variable | GFTLSSYSMNWVRQAPGKGLEWVSY | |
| | | ISSSSSTIYYADSVKGRFTISKDNA | |
| | | KNSLYLQMNSLRAEDTAVYYCAVGR | |
| | | GYFVYWGQGTLVTVSS | |
| | HCDR1 | SYSMN | 126 |
| | HCDR2 | YISSSSSTIYYADSVKG | 127 |
| | HCDR3 | GRGYFVY | 128 |
| | HFR1 | EVQLVESGGGLVQPGGSLRLSCAAS | 129 |
| | | GFTLS | |
| | HFR2 | WVRQAPGKGLEWVS | 130 |
| | HFR3 | RFTISKDNAKNSLYLQMNSLRAEDT | 131 |
| | | AVYYCAV | |
| | HFR4 | WGQGTLVTVSS | 60 |
| | LC | EIVLTQSPGTLSLSPGERATLSCRA | 132 |
| | | SQSVSSGYLAWYQQKPGQAPRLLIF | |
| | | GASSRATGIPDRFSGSGSGTDFTLT | |
| | | INRLEPEDFAVYYCQQYGSSRTFGQ | |
| | | GTKVEIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | EIVLTQSPGTLSLSPGERATLSCRA | 133 |
| | variable | SQSVSSGYLAWYQQKPGQAPRLLIF | |
| | | GASSRATGIPDRFSGSGSGTDFTLT | |
| | | INRLEPEDFAVYYCQQYGSSRTFGQ | |
| | | GTKVEIK | |
| | LCDR1 | RASQSVSSGYLA | 134 |
| | LCDR2 | GASSRAT | 135 |
| | LCDR3 | QQYGSSRT | 136 |
| | LFR1 | EIVLTQSPGTLSLSPGERATLSC | 50 |
| | LFR2 | WYQQKPGQAPRLLIF | 137 |
| | LFR3 | GIPDRFSGSGSGTDFTLTINRLEPE | 138 |
| | | DFAVYYC | |
| | LFR4 | FGQGTKVEIK | 53 |
| S24-178 | HC | QVQLVESGGGVVQPGRSLRLSCAAS | 139 |
| | | GFTFSSYGMHWVRQAPGKGLEWVAV | |
| | | IWYDGSNKYYADSVKGRFTISRDNS | |
| | | KNTLYLQMNSLRAEDTAVYYCARIE | |
| | | GYSYGDVRVYYYYGMDVWGQGTTVT | |
| | | VSSASTKGPSVFPLAPSSKSTSGGT | |
| | | AALGCLVKDYFPEPVTVSWNSGALT | |
| | | SGVHTFPAVLQSSG | |
| | HC | QVQLVESGGGVVQPGRSLRLSCAAS | 140 |
| | variable | GFTFSSYGMHWVRQAPGKGLEWVAV | |
| | | IWYDGSNKYYADSVKGRFTISRDNS | |
| | | KNTLYLQMNSLRAEDTAVYYCARIE | |
| | | GYSYGDVRVYYYYGMDVWGQGTTVT | |
| | | VSS | |
| | HCDR1 | SYGMH | 141 |
| | HCDR2 | VIWYDGSNKYYADSVKG | 142 |
| | HCDR3 | IEGYSYGDVRVYYYYGMDV | 143 |
| | HFR1 | QVQLVESGGGVVQPGRSLRLSCAAS | 144 |
| | | GFTFS | |
| | HFR2 | WVRQAPGKGLEWVA | 145 |
| | HFR3 | RFTISRDNSKNTLYLQMNSLRAEDT | 146 |
| | | AVYYCAR | |
| | HFR4 | WGQGTTVTVSS | 147 |
| | LC | QSALTQPASVSGSPGQSITISCTGT | 148 |
| | | TSDVGGYDYVSWYQQHPGKAPKLIL | |
| | | SEVSNRPSGVSNRFSGSKSGNTASL | |
| | | TISGLQAEDEADYYCSSYPSSSTLV | |
| | | FGTGTKVTVLGQPKANPTVTLFPPS | |
| | | SEELQANKATLVCLISDFYPGAVTV | |
| | | AWKADGSPVKAGVETTTPSKQSNNK | |
| | | YAASS | |
| | LC | QSALTQPASVSGSPGQSITISCTGT | 149 |
| | variable | TSDVGGYDYVSWYQQHPGKAPKLIL | |
| | | SEVSNRPSGVSNRFSGSKSGNTASL | |
| | | TISGLQAEDEADYYCSSYPSSSTLV | |
| | | FGTGTKVTVL | |
| | LCDR1 | TGTTSDVGGYDYVS | 150 |
| | LCDR2 | EVSNRPS | 151 |
| | LCDR3 | SSYPSSSTLV | 152 |
| | LFR1 | QSALTQPASVSGSPGQSITISC | 66 |
| | LFR2 | WYQQHPGKAPKLILS | 153 |
| | LFR3 | GVSNRFSGSKSGNTASLTISGLQAE | 68 |
| | | DEADYYC | |
| | LFR4 | FGTGTKVTVL | 18 |
| S24-188 | HC | QVHLVQSGAEVKKPGSSVKVSCKAS | 154 |
| | | GGTFSSCAISWVRQAPGQGLEWMGR | |
| | | IIPILGIANYAQKFQGRVTITADKS | |
| | | TSTAYMELSSLRSEDTAVYYCARGW | |
| | | EFGSGSYYRTDYYYYAMDVWGQGTT | |
| | | VTVSSASTKGPSVFPLAPCSRSTSG | |
| | | GTAALGCLVKDYFPEPVTVSWNSGA | |
| | | LTSGVHTFPAVLQSSG | |
| | HC | QVHLVQSGAEVKKPGSSVKVSCKAS | 155 |
| | variable | GGTFSSCAISWVRQAPGQGLEWMGR | |
| | | IIPILGIANYAQKFQGRVTITADKS | |
| | | TSTAYMELSSLRSEDTAVYYCARGW | |
| | | EFGSGSYYRTDYYYYAMDVWGQGTT | |
| | | VTVSS | |
| | HCDR1 | SCAIS | 156 |
| | HCDR2 | RIIPILGIANYAQKFQG | 157 |
| | HCDR3 | GWEFGSGSYYRTDYYYYAMDV | 158 |
| | HFR1 | QVHLVQSGAEVKKPGSSVKVSCKAS | 159 |
| | | GGTFS | |
| | HFR2 | WVRQAPGQGLEWMG | 160 |
| | HFR3 | RVTITADKSTSTAYMELSSLRSEDT | 161 |
| | | AVYYCAR | |
| | HFR4 | WGQGTTVTVSS | 147 |
| | LC | QSALTQPASVSGSPGQSITISCTGT | 162 |
| | | SSDVGGYNYVSWYQQHPGKAPKLMI | |
| | | YEVTNRPSGVSNRFSGSRSGNTASL | |
| | | TISGLQAEDEADYYCSSYTSSSLYV | |
| | | FGTGTKVAVLGQPKANPTVTLFPPS | |
| | | SEELQANKATLVCLISDFYPGAVTV | |
| | | AWKADSSPVKAGVETTKPSKQSNNK | |
| | | YAASS | |
| | LC | QSALTQPASVSGSPGQSITISCTGT | 163 |
| | variable | SSDVGGYNYVSWYQQHPGKAPKLMI | |
| | | YEVTNRPSGVSNRFSGSRSGNTASL | |
| | | TISGLQAEDEADYYCSSYTSSSLYV | |
| | | FGTGTKVAVL | |
| | LCDR1 | TGTSSDVGGYNYVS | 63 |
| | LCDR2 | EVTNRPS | 164 |
| | LCDR3 | SSYTSSSLYV | 165 |
| | LFR1 | QSALTQPASVSGSPGQSITISC | 66 |
| | LFR2 | WYQQHPGKAPKLMIY | 67 |
| | LFR3 | GVSNRFSGSRSGNTASLTISGLQAE | 166 |
| | | DEADYYC | |
| | LFR4 | FGTGTKVAVL | 167 |
| S24-202 | HC | EVQLVQSGAEVKKPGESLRISCKGS | 168 |
| | | GYSFSSYWISWVRQMPGKGLEWMGR | |
| | | IDPSDSNTNYSPSFQGHVTISADKS | |
| | | ISTAYLQWSSLKASDTAMYYCARLS | |
| | | VRVWFGELPHYGMDVWGQGTTVTVS | |
| | | SASTKGPSVFPLAPSSKSTSGGTAA | |
| | | LGCLVKDYFPEPVTVSWNSGALTSG | |
| | | VHTFPAVLQSSG | |
| | HC | EVQLVQSGAEVKKPGESLRISCKGS | 169 |
| | variable | GYSFSSYWISWVRQMPGKGLEWMGR | |
| | | IDPSDSNTNYSPSFQGHVTISADKS | |
| | | ISTAYLQWSSLKASDTAMYYCARLS | |
| | | VRVWFGELPHYGMDVWGQGTTVTVS | |
| | | S | |
| | HCDR1 | SYWIS | 170 |
| | HCDR2 | RIDPSDSNTNYSPSFQG | 171 |
| | HCDR3 | LSVRVWFGELPHYGMDV | 172 |
| | HFR1 | EVQLVQSGAEVKKPGESLRISCKGS | 173 |
| | | GYSFS | |
| | HFR2 | WVRQMPGKGLEWMG | 174 |
| | HFR3 | HVTISADKSISTAYLQWSSLKASDT | 175 |
| | | AMYYCAR | |
| | HFR4 | WGQGTTVTVSS | 147 |
| | LC | EIVLTQSPATLSLSPGERATLSCRA | 176 |
| | | SQSVSSYLAWYQQKPGQAPRLLIYD | |
| | | ASNRASGIPARFSGSGSGTDFTLTI | |
| | | SSLEPEDFAVYYCQQRRNWPLTFGG | |
| | | GTKVETKRTVAAPSVFIFPPSDEQL | |
| | | KSGTASVVCLLNNFYPREAKVQWKV | |
| | | DN | |
| | LC | EIVLTQSPATLSLSPGERATLSCRA | 177 |
| | variable | SQSVSSYLAWYQQKPGQAPRLLIYD | |
| | | ASNRASGIPARFSGSGSGTDFTLTI | |
| | | SSLEPEDFAVYYCQQRRNWPLTFGG | |
| | | GTKVETK | |
| | LCDR1 | RASQSVSSYLA | 178 |
| | LCDR2 | DASNRAS | 179 |
| | LCDR3 | QQRRNWPLT | 180 |
| | LFR1 | EIVLTQSPATLSLSPGERATLSC | 181 |
| | LFR2 | WYQQKPGQAPRLLIY | 182 |
| | LFR3 | GIPARFSGSGSGTDFTLTISSLEPE | 183 |
| | | DFAVYYC | |
| | LFR4 | FGGGTKVETK | 184 |
| S24-278 | HC | QVQLVQSGAEVKKPGASVKVSCKAS | 185 |
| | | GYTFTGYYMHWVRQAPGQGLEWMGW | |
| | | INPNSGDTNYAQKFQGWVTMTRDTS | |
| | | LSTAYMELSRLKSDDTAVYYCARVG | |
| | | VGEYSGRHYYYYGMDVWGQGTTVTV | |
| | | SSASTKGPSVFPLAPSSKSTSGGTA | |
| | | ALGCLVKDYFPEPVTVSWNSGALTS | |
| | | GVHTFPAVLQSSG | |
| | HC | QVQLVQSGAEVKKPGASVKVSCKAS | 186 |
| | variable | GYTFTGYYMHWVRQAPGQGLEWMGW | |
| | | INPNSGDTNYAQKFQGWVTMTRDTS | |
| | | LSTAYMELSRLKSDDTAVYYCARVG | |
| | | VGEYSGRHYYYYGMDVWGQGTTVTV | |
| | | SS | |
| | HCDR1 | GYYMH | 187 |
| | HCDR2 | WINPNSGDTNYAQKFQG | 188 |
| | HCDR3 | VGVGEYSGRHYYYYGMDV | 189 |
| | HFR1 | QVQLVQSGAEVKKPGASVKVSCKAS | 190 |
| | | GYTFT | |
| | HFR2 | WVRQAPGQGLEWMG | 160 |
| | HFR3 | WVTMTRDTSLSTAYMELSRLKSDDT | 191 |
| | | AVYYCAR | |
| | HFR4 | WGQGTTVTVSS | 147 |
| | LC | EIVLTQSPGTLSLSPGERATLSCRA | 192 |
| | | SQSISSSYLAWYQQKPGQAPRLLIY | |
| | | GASSRATGIPDRFSGSGSGTDFTLT | |
| | | ISRLEPEDFAVYYCQQYGSSLTFGG | |
| | | GTKVEIKRTVAAPSVFIFPPSDEQL | |
| | | KSGTASVVCLLNNFYPREAKVQWKV | |
| | | DN | |
| | LC | EIVLTQSPGTLSLSPGERATLSCRA | 193 |
| | variable | SQSISSSYLAWYQQKPGQAPRLLIY | |
| | | GASSRATGIPDRFSGSGSGTDFTLT | |
| | | ISRLEPEDFAVYYCQQYGSSLTFGG | |
| | | GTKVEIK | |
| | LCDR1 | RASQSISSSYLA | 194 |
| | LCDR2 | GASSRAT | 135 |
| | LCDR3 | QQYGSSLT | 195 |
| | LFR1 | EIVLTQSPGTLSLSPGERATLSC | 50 |
| | LFR2 | WYQQKPGQAPRLLIY | 182 |
| | LFR3 | GIPDRFSGSGSGTDFTLTISRLEPE | 196 |
| | | DFAVYYC | |
| | LFR4 | FGGGTKVEIK | 85 |
| S24-339 | HC | EVQLVESGGGLVQPGRSLRLSCTAS | 197 |
| | | GFTFGDYAMSWFRQAPGKGLEWVGF | |
| | | IRSKAYGGTTQHAASVKGRFTISRD | |
| | | DSKSIAYLQMNSLKTEDTAVYHCAR | |
| | | DGYDCSGGRCYSHIFDYWGQGTLVT | |
| | | VSSGESSPPPLVHLGRLSLPGSQGQ | |
| | | SLV | |
| | HC | EVQLVESGGGLVQPGRSLRLSCTAS | 198 |
| | variable | GFTFGDYAMSWFRQAPGKGLEWVGF | |
| | | IRSKAYGGTTQHAASVKGRFTISRD | |
| | | DSKSIAYLQMNSLKTEDTAVYHCAR | |
| | | DGYDCSGGRCYSHIFDYWGQGTLVT | |
| | | VSS | |
| | HCDR1 | DYAMS | 199 |
| | HCDR2 | FIRSKAYGGTTQHAASVKG | 200 |
| | HCDR3 | DGYDCSGGRCYSHIFDY | 201 |
| | HFR1 | EVQLVESGGGLVQPGRSLRLSCTAS | 202 |
| | | GFTFG | |
| | HFR2 | WFRQAPGKGLEWVG | 203 |
| | HFR3 | RFTISRDDSKSIAYLQMNSLKTEDT | 204 |
| | | AVYHCAR | |
| | HFR4 | WGQGTLVTVSS | 60 |
| | LC | EIVMTQSPATLSVSPGERATLSCRA | 205 |
| | | SQSVSSNLAWYQQKPGQAPRLLIYG | |
| | | ASTRATGIPARFSGSGSGTEFTLTI | |
| | | SSLQSEDFAVYYCQQYDNWWTFGQG | |
| | | TKVEIKRTVAAPSVFIFPPSDEQLK | |
| | | SGTASVVCLLNNFYPREAKVQWKVD | |
| | | N | |
| | LC | EIVMTQSPATLSVSPGERATLSCRA | 206 |
| | variable | SQSVSSNLAWYQQKPGQAPRLLIYG | |
| | | ASTRATGIPARFSGSGSGTEFTLTI | |
| | | SSLQSEDFAVYYCQQYDNWWTFGQG | |
| | | TKVEIK | |
| | LCDR1 | RASQSVSSNLA | 207 |
| | LCDR2 | GASTRAT | 208 |
| | LCDR3 | QQYDNWWT | 209 |
| | LFR1 | EIVMTQSPATLSVSPGERATLSC | 210 |
| | LFR2 | WYQQKPGQAPRLLIY | 182 |
| | LFR3 | GIPARFSGSGSGTEFTLTISSLQSE | 211 |
| | | DFAVYYC | |
| | LFR4 | FGQGTKVEIK | 53 |
| S24-472 | HC | QVQLQESGPGLVKPSGTLSLTCAVS | 212 |
| | | GGSISSINWWSWVRQPPGKGLEWIG | |
| | | EIYHSGNTNYNPSLKSRVTISGDKS | |
| | | KNQFSLKLSSVTAADTAVYYCARGY | |
| | | YDSSPYYEPQGIDYWGQGILVTVSS | |
| | | ASTKGPSVFPLAPSSKSTSGGTAAL | |
| | | GCLVKDYFPEPVTVSWNSGALTSGV | |
| | | HTFPAVLQSSG | |
| | HC | QVQLQESGPGLVKPSGTLSLTCAVS | 213 |
| | variable | GGSISSINWWSWVRQPPGKGLEWIG | |
| | | EIYHSGNTNYNPSLKSRVTISGDKS | |
| | | KNQFSLKLSSVTAADTAVYYCARGY | |
| | | YDSSPYYEPQGIDYWGQGILVTVSS | |
| | HCDR1 | SINWWS | 214 |
| | HCDR2 | EIYHSGNTNYNPSLKS | 215 |
| | HCDR3 | GYYDSSPYYEPQGIDY | 216 |
| | HFR1 | QVQLQESGPGLVKPSGTLSLTCAVS | 217 |
| | | GGSIS | |
| | HFR2 | WVRQPPGKGLEWIG | 218 |
| | HFR3 | RVTISGDKSKNQFSLKLSSVTAADT | 219 |
| | | AVYYCAR | |
| | HFR4 | WGQGILVTVSS | 220 |
| | LC | QLVLTQSPSASASLGASVKLTCTLS | 221 |
| | | SGHSSYTIAWHQQQPEKGPRYLMKV | |
| | | NSDGSHTKGDGIPDRFSGSSSGAER | |
| | | YLTISSLQSEDEADYYCQTWGTGIR | |
| | | VFGGGTKLTVLGQPKAAPSVTLFPP | |
| | | SSEELQANKATLVCLISDFYPGAVT | |
| | | VAWKADSSPVKAGVETTTPSKQSNN | |
| | | KYAASS | |
| | LC | QLVLTQSPSASASLGASVKLTCTLS | 222 |
| | variable | SGHSSYTIAWHQQQPEKGPRYLMKV | |
| | | NSDGSHTKGDGIPDRFSGSSSGAER | |
| | | YLTISSLQSEDEADYYCQTWGTGIR | |
| | | VFGGGTKLTVL | |
| | LCDR1 | TLSSGHSSYTIA | 223 |
| | LCDR2 | VNSDGSHTKGD | 224 |
| | LCDR3 | QTWGTGIRV | 225 |
| | LFR1 | QLVLTQSPSASASLGASVKLTC | 226 |
| | LFR2 | WHQQQPEKGPRYLMK | 227 |
| | LFR3 | GIPDRFSGSSSGAERYLTISSLQSE | 228 |
| | | DEADYYC | |
| | LFR4 | FGGGTKLTVL | 69 |
| S24-490 | HC | QVQLVQSGAEVKKPGASVKVSCKAS | 229 |
| | | GYTFTSYFIHWVRQAPGQGLEWMGI | |
| | | INPSGGSTSYAQKFQGRVTMTRDTS | |
| | | TSTVYMELSSLRSEDTAVYYCARHT | |
| | | TPTRYFDYWGQGTLVTVSSGSASAP | |
| | | TLFPLVSCENSPSDTSSV | |
| | HC | QVQLVQSGAEVKKPGASVKVSCKAS | 230 |
| | variable | GYTFTSYFIHWVRQAPGQGLEWMGI | |
| | | INPSGGSTSYAQKFQGRVTMTRDTS | |
| | | TSTVYMELSSLRSEDTAVYYCARHT | |
| | | TPTRYFDYWGQGTLVTVSS | |
| | HCDR1 | SYFIH | 231 |
| | HCDR2 | IINPSGGSTSYAQKFQG | 232 |
| | HCDR3 | HTTPTRYFDY | 233 |
| | HFR1 | QVQLVQSGAEVKKPGASVKVSCKAS | 190 |
| | | GYTFT | |
| | HFR2 | WVRQAPGQGLEWMG | 160 |
| | HFR3 | RVTMTRDTSTSTVYMELSSLRSEDT | 234 |
| | | AVYYCAR | |
| | HFR4 | WGQGTLVTVSS | 60 |
| | LC | EIVLTQSPGTLSLSPGERATLSCRA | 235 |
| | | SQSVTSSYLAWYQQRRGQAPRLLIY | |
| | | GASSRATGIPDRFSGSGSGTDFTLT | |
| | | ISRLEPEDFAVYYCQQYGSSPLTFG | |
| | | GGTKVEIKRTVAAPSVFIFPPSDEQ | |
| | | LKSGTASVVCLLNNFYPREAKVQWK | |
| | | VDN | |
| | LC | EIVLTQSPGTLSLSPGERATLSCRA | 236 |
| | variable | SQSVTSSYLAWYQQRRGQAPRLLIY | |
| | | GASSRATGIPDRFSGSGSGTDFTLT | |
| | | ISRLEPEDFAVYYCQQYGSSPLTFG | |
| | | GGTKVEIK | |
| | LCDR1 | RASQSVTSSYLA | 237 |
| | LCDR2 | GASSRAT | 135 |
| | LCDR3 | QQYGSSPLT | 238 |
| | LFR1 | EIVLTQSPGTLSLSPGERATLSC | 50 |
| | LFR2 | WYQQRRGQAPRLLIY | 239 |
| | LFR3 | GIPDRFSGSGSGTDFTLTISRLEPE | 196 |
| | | DFAVYYC | |
| | LFR4 | FGGGTKVEIK | 85 |
| S24-494 | HC | QLQLQESGPGLVKPSETLSLTCTVS | 240 |
| | | GGSISSSSYYWGWIRQPPGKGLEWI | |
| | | GSIYYSGSTYYNPSLKSRVTISVDT | |
| | | SKNQFSLKLSSVTAADTAVYYCARK | |
| | | PRSDYGYFDLWGRGTLVTVSSASTK | |
| | | GPSV | |
| | HC | QLQLQESGPGLVKPSETLSLTCTVS | 241 |
| | variable | GGSISSSSYYWGWIRQPPGKGLEWI | |
| | | GSIYYSGSTYYNPSLKSRVTISVDT | |
| | | SKNQFSLKLSSVTAADTAVYYCARK | |
| | | PRSDYGYFDLWGRGTLVTVSS | |
| | HCDR1 | SSSYYWG | 242 |
| | HCDR2 | SIYYSGSTYYNPSLKS | 243 |
| | HCDR3 | KPRSDYGYFDL | 244 |
| | HFR1 | QLQLQESGPGLVKPSETLSLTCTVS | 245 |
| | | GGSIS | |
| | HFR2 | WIRQPPGKGLEWIG | 7 |
| | HFR3 | RVTISVDTSKNQFSLKLSSVTAADT | 115 |
| | | AVYYCAR | |
| | HFR4 | WGRGTLVTVSS | 9 |
| | LC | DIQMTQSPSSLSASVGDRVTITCRA | 246 |
| | | SQSISSYLNWYQQKPGKAPKLLIYA | |
| | | ASSLQSGVPSRFSGSGSGTDFTLTI | |
| | | SSLQPEDFATYYCQQSYSTPQLTFG | |
| | | GGTKVEIKRTVAAPSVFIFPPSDEQ | |
| | | LKSGTASVVCLLNNFYPREAKVQWK | |
| | | VDN | |
| | LC | DIQMTQSPSSLSASVGDRVTITCRA | 247 |
| | variable | SQSISSYLNWYQQKPGKAPKLLIYA | |
| | | ASSLQSGVPSRFSGSGSGTDFTLTI | |
| | | SSLQPEDFATYYCQQSYSTPQLTFG | |
| | | GGTKVEIK | |
| | LCDR1 | RASQSISSYLN | 248 |
| | LCDR2 | AASSLQS | 249 |
| | LCDR3 | QQSYSTPQLT | 250 |
| | LFR1 | DIQMTQSPSSLSASVGDRVTITC | 251 |
| | LFR2 | WYQQKPGKAPKLLIY | 252 |
| | LFR3 | GVPSRFSGSGSGTDFTLTISSLQPE | 253 |
| | | DFATYYC | |
| | LFR4 | FGGGTKVEIK | 85 |
| S24-566 | HC | EVQLVESGGGLVKPGRSLRLSCTAS | 254 |
| | | GFTFGDYAMSWFRQAPGKGLEWVGF | |
| | | TRRKAYGGTTEYAASVKGRFTISRD | |
| | | DSKSIAYLQMNSLKTEDTAVYYCTR | |
| | | IKVGRFDLTDSGSYRYFDYWGQGTL | |
| | | VTVSSASTKGPSVFPLAPSSKSTSG | |
| | | GTAALGCLVKDYFPEPVTVSWNSGA | |
| | | LTSGVHTFPAVLQSSG | |
| | HC | EVQLVESGGGLVKPGRSLRLSCTAS | 255 |
| | variable | GFTFGDYAMSWFRQAPGKGLEWVGF | |
| | | TRRKAYGGTTEYAASVKGRFTISRD | |
| | | DSKSIAYLQMNSLKTEDTAVYYCTR | |
| | | IKVGRFDLTDSGSYRYFDYWGQGTL | |
| | | VTVSS | |
| | HCDR1 | DYAMS | 199 |
| | HCDR2 | FTRRKAYGGTTEYAASVKG | 256 |
| | HCDR3 | IKVGRFDLTDSGSYRYFDY | 257 |
| | HFR1 | EVQLVESGGGLVKPGRSLRLSCTAS | 258 |
| | | GFTFG | |
| | HFR2 | WFRQAPGKGLEWVG | 203 |
| | HFR3 | RFTISRDDSKSIAYLQMNSLKTEDT | 259 |
| | | AVYYCTR | |
| | HFR4 | WGQGTLVTVSS | 60 |
| | LC | DIVMTQSPLSLPVTPGEPASISCRS | 260 |
| | | SQSLLHSNGYNYLDWYLQKPGQSPQ | |
| | | LLIYLGSNRASGVPDRFSGSGSGTD | |
| | | FTLKISRVEAEDVGVYYCMQPLQTP | |
| | | WTFGQGTKVEIKRTVAAPSVFIFPP | |
| | | SDEQLKSGTASVVCLLNNFYPREAK | |
| | | VQWKVDN | |
| | LC | DIVMTQSPLSLPVTPGEPASISCRS | 261 |
| | variable | SQSLLHSNGYNYLDWYLQKPGQSPQ | |
| | | LLIYLGSNRASGVPDRFSGSGSGTD | |
| | | FTLKISRVEAEDVGVYYCMQPLQTP | |
| | | WTFGQGTKVEIK | |
| | LCDR1 | RSSQSLLHSNGYNYLD | 262 |
| | LCDR2 | LGSNRAS | 263 |
| | LCDR3 | MQPLQTPWT | 264 |
| | LFR1 | DIVMTQSPLSLPVTPGEPASISC | 265 |
| | LFR2 | WYLQKPGQSPQLLIY | 266 |
| | LFR3 | GVPDRFSGSGSGTDFTLKISRVEAE | 267 |
| | | DVGVYYC | |
| | LFR4 | FGQGTKVEIK | 53 |
| S24-636 | HC | EVQLVESGGGLVQPGGSLRLSCAAS | 268 |
| | | GFTLSSYWMSWVRQAPGKGLEWVAN | |
| | | IKQDGSEKYYVDSVKGRFTISRDNA | |
| | | KNSLYLQMNSLRAEDTAVYYCARDL | |
| | | TATWFDPWGQGTLVTVSSAPTKAPD | |
| | | VFPIISGCRHPKDNSPVVLACLITG | |
| | | YH | |
| | HC | EVQLVESGGGLVQPGGSLRLSCAAS | 269 |
| | variable | GFTLSSYWMSWVRQAPGKGLEWVAN | |
| | | IKQDGSEKYYVDSVKGRFTISRDNA | |
| | | KNSLYLQMNSLRAEDTAVYYCARDL | |
| | | TATWFDPWGQGTLVTVSS | |
| | HCDR1 | SYWMS | 270 |
| | HCDR2 | NIKQDGSEKYYVDSVKG | 271 |
| | HCDR3 | DLTATWFDP | 272 |
| | HFR1 | EVQLVESGGGLVQPGGSLRLSCAAS | 129 |
| | | GFTLS | |
| | HFR2 | WVRQAPGKGLEWVA | 145 |
| | HFR3 | RFTISRDNAKNSLYLQMNSLRAEDT | 273 |
| | | AVYYCAR | |
| | HFR4 | WGQGTLVTVSS | 60 |
| | LC | QTVVTQEPSFSVSPGGTVTLTCGLS | 274 |
| | | SGSVSTSYYPSWYQQTPGQAPRTLI | |
| | | YSTNKRSSGVPDRFSGSILGNKAAL | |
| | | TITGAQADDESDYYCVLYMGSGMSV | |
| | | FGGGTKLTVLGQPKAAPSVTLFPPS | |
| | | SEELQANKATLVCLISDFYPGAVTV | |
| | | AWKADSSPVKAGVETTTPSKQSNNK | |
| | | YAASS | |
| | LC | QTVVTQEPSFSVSPGGTVTLTCGLS | 275 |
| | variable | SGSVSTSYYPSWYQQTPGQAPRTLI | |
| | | YSTNKRSSGVPDRFSGSILGNKAAL | |
| | | TITGAQADDESDYYCVLYMGSGMSV | |
| | | FGGGTKLTVL | |
| | LCDR1 | GLSSGSVSTSYYPS | 276 |
| | LCDR2 | STNKRSS | 277 |
| | LCDR3 | VLYMGSGMSV | 278 |
| | LFR1 | QTVVTQEPSFSVSPGGTVTLTC | 279 |
| | LFR2 | WYQQTPGQAPRTLIY | 280 |
| | LFR3 | GVPDRFSGSILGNKAALTITGAQAD | 281 |
| | | DESDYYC | |
| | LFR4 | FGGGTKLTVL | 69 |
| S24-740 | HC | QVQLVQSGAEVKKPGASVKVSCKAS | 282 |
| | | GYTFTSYALHWVRQAPGQRLEWMGW | |
| | | INAGNGNTKYSQRFQGRVTIIRDTS | |
| | | ASTTYMELSSLRSEDTAVYYCARGY | |
| | | ARAGVITIKESLHHWGQGTLVTVSS | |
| | | ASTKGPSVFPLAPSSKSTSGGTAAL | |
| | | GCLVKDYFPEPVTVSWNSGALTSGV | |
| | | HTFPAVLQSSG | |
| | HC | QVQLVQSGAEVKKPGASVKVSCKAS | 283 |
| | variable | GYTFTSYALHWVRQAPGQRLEWMGW | |
| | | INAGNGNTKYSQRFQGRVTIIRDTS | |
| | | ASTTYMELSSLRSEDTAVYYCARGY | |
| | | ARAGVITIKESLHHWGQGTLVTVSS | |
| | HCDR1 | SYALH | 284 |
| | HCDR2 | WINAGNGNTKYSQRFQG | 285 |
| | HCDR3 | GYARAGVITIKESLHH | 286 |
| | HFR1 | QVQLVQSGAEVKKPGASVKVSCKAS | 190 |
| | | GYTFT | |
| | HFR2 | WVRQAPGQRLEWMG | 287 |
| | HFR3 | RVTIIRDTSASTTYMELSSLRSEDT | 288 |
| | | AVYYCAR | |
| | HFR4 | WGQGTLVTVSS | 60 |
| | LC | DIVMTQSPDSLAVSLGERATINCKS | 289 |
| | | SQSVLYSSNNKNYLAWYQQKPGQPP | |
| | | KLLIYWASTRESGVPDRFSGSGSGT | |
| | | DFTLTISSLQAEDVAVYYCQQYYST | |
| | | PPLTFGGGTKVEIKRTVAAPSVFIF | |
| | | PPSDEQLKSGTASVVCLLNNFYPRE | |
| | | AKVQWKVDN | |
| | LC | DIVMTQSPDSLAVSLGERATINCKS | 290 |
| | variable | SQSVLYSSNNKNYLAWYQQKPGQPP | |
| | | KLLIYWASTRESGVPDRFSGSGSGT | |
| | | DFTLTISSLQAEDVAVYYCQQYYST | |
| | | PPLTFGGGTKVEIK | |
| | LCDR1 | KSSQSVLYSSNNKNYLA | 291 |
| | LCDR2 | WASTRES | 30 |
| | LCDR3 | QQYYSTPPLT | 292 |
| | LFR1 | DIVMTQSPDSLAVSLGERATINC | 32 |
| | LFR2 | WYQQKPGQPPKLLIY | 33 |
| | LFR3 | GVPDRFSGSGSGTDFTLTISSLQAE | 293 |
| | | DVAVYYC | |
| | LFR4 | FGGGTKVEIK | 85 |
| S24-791 | HC | QVQLQESGPGLVKPSETLSLTCTVS | 294 |
| | | GGSISSSYWSWIRQPPGKGLEWIGY | |
| | | IYYSGNTNYNPSLKSRVTLSIDTSK | |
| | | NQFSLKLSSVTAADTAVYYCACSVT | |
| | | IFGVVTPAFDIWGQGTMVTVSSAST | |
| | | KGPSVFPLAPSSKSTSGGTAALGCL | |
| | | VKDYFPEPVTVSWNSGALTSGVHTF | |
| | | PAVLQSSG | |
| | HC | QVQLQESGPGLVKPSETLSLTCTVS | 295 |
| | variable | GGSISSSYWSWIRQPPGKGLEWIGY | |
| | | IYYSGNTNYNPSLKSRVTLSIDTSK | |
| | | NQFSLKLSSVTAADTAVYYCACSVT | |
| | | IFGVVTPAFDIWGQGTMVTVSS | |
| | HCDR1 | SSYWS | 296 |
| | HCDR2 | YIYYSGNTNYNPSLKS | 297 |
| | HCDR3 | SVTIFGVVTPAFDI | 298 |
| | HFR1 | QVQLQESGPGLVKPSETLSLTCTVS | 24 |
| | | GGSIS | |
| | HFR2 | WIRQPPGKGLEWIG | 7 |
| | HFR3 | RVTLSIDTSKNQFSLKLSSVTAADT | 299 |
| | | AVYYCAC | |
| | HFR4 | WGQGTMVTVSS | 44 |
| | LC | EIVLTHSPGTLSLSPGERATLSCRA | 300 |
| | | SQSVRSYLAWYQQKPGQAPRLLIYG | |
| | | ASSRATGIPDRFSGSGSGTDFTLTI | |
| | | SRLEPDDFAVYYCQQYGSSPWTFGQ | |
| | | GTKVEIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | EIVLTHSPGTLSLSPGERATLSCRA | 301 |
| | variable | SQSVRSYLAWYQQKPGQAPRLLIYG | |
| | | ASSRATGIPDRFSGSGSGTDFTLTI | |
| | | SRLEPDDFAVYYCQQYGSSPWTFGQ | |
| | | GTKVEIK | |
| | LCDR1 | RASQSVRSYLA | 302 |
| | LCDR2 | GASSRAT | 135 |
| | LCDR3 | QQYGSSPWT | 303 |
| | LFR1 | EIVLTHSPGTLSLSPGERATLSC | 304 |
| | LFR2 | WYQQKPGQAPRLLIY | 182 |
| | LFR3 | GIPDRFSGSGSGTDFTLTISRLEPD | 305 |
| | | DFAVYYC | |
| | LFR4 | FGQGTKVEIK | 53 |
| S24-902 | HC | QVQLVQSGAEVKKPGSSVKVSCKAS | 306 |
| | | GGTFSSYAISWVRQAPGQGLEWMGR | |
| | | IIPILGIANYAQKFQGRVTITADKS | |
| | | TSTAYMELSSLRSEDTAVYYCARWD | |
| | | FGVVIQYGMDVWGQGTTVTVSSAST | |
| | | KGPSVFPLAPSSKSTSGGTAALGCL | |
| | | VKDYFPEPVTVSWNSGALTSGVHTF | |
| | | PAVLQSSGLYSLSSVVTVPSSSL | |
| | HC | QVQLVQSGAEVKKPGSSVKVSCKAS | 307 |
| | variable | GGTFSSYAISWVRQAPGQGLEWMGR | |
| | | IIPILGIANYAQKFQGRVTITADKS | |
| | | TSTAYMELSSLRSEDTAVYYCARWD | |
| | | FGVVIQYGMDVWGQGTTVTVSS | |
| | HCDR1 | SYAIS | 308 |
| | HCDR2 | RIIPILGIANYAQKFQG | 157 |
| | HCDR3 | WDFGVVIQYGMDV | 309 |
| | HFR1 | QVQLVQSGAEVKKPGSSVKVSCKAS | 310 |
| | | GGTFS | |
| | HFR2 | WVRQAPGQGLEWMG | 160 |
| | HFR3 | RVTITADKSTSTAYMELSSLRSEDT | 161 |
| | | AVYYCAR | |
| | HFR4 | WGQGTTVTVSS | 147 |
| | LC | QAVVTQEPSLTVSPGGTVTLTCGSS | 311 |
| | | TGAVTSGHYPYWFQQKPGQAPRTLI | |
| | | YDTSNKHSWTPARFSGSLLGGKAAL | |
| | | TLSGAQPEDEAEYYCLLSYSGWVFG | |
| | | GGTKLTVLGQPKAAPSVTLFPPSSE | |
| | | ELQANKATLVCLISDFYPGAVTVAW | |
| | | KADSSPVKAGVETTTPSKQSNNKYA | |
| | | ASS | |
| | LC | QAVVTQEPSLTVSPGGTVTLTCGSS | 312 |
| | variable | TGAVTSGHYPYWFQQKPGQAPRTLI | |
| | | YDTSNKHSWTPARFSGSLLGGKAAL | |
| | | TLSGAQPEDEAEYYCLLSYSGWVFG | |
| | | GGTKLTVL | |
| | LCDR1 | GSSTGAVTSGHYPY | 313 |
| | LCDR2 | DTSNKHS | 314 |
| | LCDR3 | LLSYSGWV | 315 |
| | LFR1 | QAVVTQEPSLTVSPGGTVTLTC | 316 |
| | LFR2 | WFQQKPGQAPRTLIY | 317 |
| | LFR3 | WTPARFSGSLLGGKAALTLSGAQPE | 318 |
| | | DEAEYYC | |
| | LFR4 | FGGGTKLTVL | 69 |
| S24-921 | HC | QVQLQESGPGLVKPSETLSLTCTVS | 319 |
| | | GGSINSFYWNWIRQPPGKGLEWIGY | |
| | | IYYSGNTKYNPSLKSRVTISVDTSN | |
| | | SQFSLKLSSVTAADTAVYYCAALKK | |
| | | QELVSLQAFDIWGQGTMVTVSSAST | |
| | | KGPSVFPLAPSSKSTSGGTAALGCL | |
| | | VKDYFPEPVTVSWNSGALTSGVHTF | |
| | | PAVLQSSG | |
| | HC | QVQLQESGPGLVKPSETLSLTCTVS | 320 |
| | variable | GGSINSFYWNWIRQPPGKGLEWIGY | |
| | | IYYSGNTKYNPSLKSRVTISVDTSN | |
| | | SQFSLKLSSVTAADTAVYYCAALKK | |
| | | QELVSLQAFDIWGQGTMVTVSS | |
| | HCDR1 | SFYWN | 321 |
| | HCDR2 | YIYYSGNTKYNPSLKS | 322 |
| | HCDR3 | LKKQELVSLQAFDI | 323 |
| | HFR1 | QVQLQESGPGLVKPSETLSLTCTVS | 324 |
| | | GGSIN | |
| | HFR2 | WIRQPPGKGLEWIG | 7 |
| | HFR3 | RVTISVDTSNSQFSLKLSSVTAADT | 325 |
| | | AVYYCAA | |
| | HFR4 | WGQGTMVTVSS | 44 |
| | LC | DIQMTQSPSSLSASLGDGVTITCRA | 326 |
| | | SQSISSYLSWYQQKPGKAPKLLIYA | |
| | | ASSLQSGVPSRFSGSGSGTDFTLTI | |
| | | SSLQPEDFATYYCQQSYNTPVTFGQ | |
| | | GTKVEIKRTVAAPSVFIFPPSDEQL | |
| | | KSGTASVVCLLNNFYPREAKVQWKV | |
| | | DNADRKS | |
| | LC | DIQMTQSPSSLSASLGDGVTITCRA | 327 |
| | variable | SQSISSYLSWYQQKPGKAPKLLIYA | |
| | | ASSLQSGVPSRFSGSGSGTDFTLTI | |
| | | SSLQPEDFATYYCQQSYNTPVTFGQ | |
| | | GTKVEIK | |
| | LCDR1 | RASQSISSYLS | 328 |
| | LCDR2 | AASSLQS | 249 |
| | LCDR3 | QQSYNTPVT | 329 |
| | LFR1 | DIQMTQSPSSLSASLGDGVTITC | 330 |
| | LFR2 | WYQQKPGKAPKLLIY | 252 |
| | LFR3 | GVPSRFSGSGSGTDFTLTISSLQPE | 253 |
| | | DFATYYC | |
| | LFR4 | FGQGTKVEIK | 53 |
| S24-1063 | HC | QVQLQESGPGLVKPSETLSLTCTVS | 331 |
| | | GGSISSYYWSWIRQPPGKGLEWIGY | |
| | | IYYSGSTKYNPSLKSRVTISVDTSK | |
| | | NQFSLKLTSVTAADTAVYYCARIYD | |
| | | SSGYYHPVFDYWGQGTLVTVSSAST | |
| | | KGPSVFPLAPSSKSTSGGTAALGCL | |
| | | VKDYFPEPVTVSWNSGALTSGVHTF | |
| | | PAVLQSSG | |
| | HC | QVQLQESGPGLVKPSETLSLTCTVS | 332 |
| | variable | GGSISSYYWSWIRQPPGKGLEWIGY | |
| | | IYYSGSTKYNPSLKSRVTISVDTSK | |
| | | NQFSLKLTSVTAADTAVYYCARIYD | |
| | | SSGYYHPVFDYWGQGTLVTVSS | |
| | HCDR1 | SYYWS | 56 |
| | HCDR2 | YIYYSGSTKYNPSLKS | 333 |
| | HCDR3 | IYDSSGYYHPVFDY | 334 |
| | HFR1 | QVQLQESGPGLVKPSETLSLTCTVS | 24 |
| | | GGSIS | |
| | HFR2 | WIRQPPGKGLEWIG | 7 |
| | HFR3 | RVTISVDTSKNQFSLKLTSVTAADT | 335 |
| | | AVYYCAR | |
| | HFR4 | WGQGTLVTVSS | 60 |
| | LC | EIVLTQSPGTLSLSPGERATLSCRA | 336 |
| | | SQSVSSSYLAWYQQKPGQAPRLLIY | |
| | | GASSRATDIPDRFSGSGSGTDFTLT | |
| | | ISRLEPEDFAVYYCQQYGSSPWTFG | |
| | | QGTKVEIKRTVAAPSVFIFPPSDEQ | |
| | | LKSGTASVVCLLNNFYPREAKVQWK | |
| | | VDN | |
| | LC | EIVLTQSPGTLSLSPGERATLSCRA | 337 |
| | variable | SQSVSSSYLAWYQQKPGQAPRLLIY | |
| | | GASSRATDIPDRFSGSGSGTDFTLT | |
| | | ISRLEPEDFAVYYCQQYGSSPWTFG | |
| | | QGTKVEIK | |
| | LCDR1 | RASQSVSSSYLA | 338 |
| | LCDR2 | GASSRAT | 135 |
| | LCDR3 | QQYGSSPWT | 303 |
| | LFR1 | EIVLTQSPGTLSLSPGERATLSC | 50 |
| | LFR2 | WYQQKPGQAPRLLIY | 182 |
| | LFR3 | DIPDRFSGSGSGTDFTLTISRLEPE | 339 |
| | | DFAVYYC | |
| | LFR4 | FGQGTKVEIK | 53 |
| S24-1224 | HC | QVQLVQSGAEVKKPGASVRVSCKAS | 340 |
| | | GYTFTSYYIYWVRQAPGQGLEWMGV | |
| | | INPSGGSTSYAQKFQGRVTLTRDTS | |
| | | TSTVYMDLSSLRSEDTAVYYCARDP | |
| | | IMWEVVTRGRGNWFDPWGQGTLVTV | |
| | | SSASTKGPSVFPLAPSSKSTSGGTA | |
| | | ALGCLVKDYFPEPVTVSWNSGALTS | |
| | | GVHTFPAVLQSSG | |
| | HC | QVQLVQSGAEVKKPGASVRVSCKAS | 341 |
| | variable | GYTFTSYYIYWVRQAPGQGLEWMGV | |
| | | INPSGGSTSYAQKFQGRVTLTRDTS | |
| | | TSTVYMDLSSLRSEDTAVYYCARDP | |
| | | IMWEVVTRGRGNWFDPWGQGTLVTV | |
| | | SS | |
| | HCDR1 | SYYIY | 342 |
| | HCDR2 | VINPSGGSTSYAQKFQG | 343 |
| | HCDR3 | DPIMWEVVTRGRGNWFDP | 344 |
| | HFR1 | QVQLVQSGAEVKKPGASVRVSCKAS | 345 |
| | | GYTFT | |
| | HFR2 | WVRQAPGQGLEWMG | 160 |
| | HFR3 | RVTLTRDTSTSTVYMDLSSLRSEDT | 346 |
| | | AVYYCAR | |
| | HFR4 | WGQGTLVTVSS | 60 |
| | LC | QSVLTQPPSVSGAPGQRVTIPCTGS | 347 |
| | | SFNIGAGYDVHWYQQLPGTAPKLLI | |
| | | FGNSNRPSGVPDRFSGSRSGTSASL | |
| | | AITGLQAEDEADYYCQSYDSSLSGV | |
| | | VFGGGTTLTVLGQPKAAPSVTLFPP | |
| | | SSEELQANKATLVCLISDFYPGAVT | |
| | | VAWKADSSPVKAGVETTTPSKQSNN | |
| | | KYAASSYLSLTPEQWKSH | |
| | LC | QSVLTQPPSVSGAPGQRVTIPCTGS | 348 |
| | variable | SFNIGAGYDVHWYQQLPGTAPKLLI | |
| | | FGNSNRPSGVPDRFSGSRSGTSASL | |
| | | AITGLQAEDEADYYCQSYDSSLSGV | |
| | | VFGGGTTLTVL | |
| | LCDR1 | TGSSFNIGAGYDVH | 349 |
| | LCDR2 | GNSNRPS | 350 |
| | LCDR3 | QSYDSSLSGVV | 351 |
| | LFR1 | QSVLTQPPSVSGAPGQRVTIPC | 352 |
| | LFR2 | WYQQLPGTAPKLLIF | 353 |
| | LFR3 | GVPDRFSGSRSGTSASLAITGLQAE | 354 |
| | | DEADYYC | |
| | LFR4 | FGGGTTLTVL | 355 |
| S24-1271 | HC | EVQLVESGGGLVQPGGSLRLSCAAS | 356 |
| | | GFTVSSNYMSWVRQAPGKGLEWVSV | |
| | | IYSDGNTYYADSVKGRFTISRDNSK | |
| | | NMLYLQMNSLRAEDTAVYYCARDPG | |
| | | QGYCSGGSCAPSYSLDYWGQGTLVT | |
| | | VSSGSASAPTLFPLVSCENSPSDTS | |
| | | SV | |
| | HC | EVQLVESGGGLVQPGGSLRLSCAAS | 357 |
| | variable | GFTVSSNYMSWVRQAPGKGLEWVSV | |
| | | IYSDGNTYYADSVKGRFTISRDNSK | |
| | | NMLYLQMNSLRAEDTAVYYCARDPG | |
| | | QGYCSGGSCAPSYSLDYWGQGTLVT | |
| | | VSS | |
| | HCDR1 | SNYMS | 358 |
| | HCDR2 | VIYSDGNTYYADSVKG | 359 |
| | HCDR3 | DPGQGYCSGGSCAPSYSLDY | 360 |
| | HFR1 | EVQLVESGGGLVQPGGSLRLSCAAS | 361 |
| | | GFTVS | |
| | HFR2 | WVRQAPGKGLEWVS | 130 |
| | HFR3 | RFTISRDNSKNMLYLQMNSLRAEDT | 362 |
| | | AVYYCAR | |
| | HFR4 | WGQGTLVTVSS | 60 |
| | LC | SYELTQPPSVSVSPGQTASITCSGD | 363 |
| | | KLGDRYVCWYQQKPGQSPVLVIYQD | |
| | | TKRPSGIPERFSGSNSGNTATLTIS | |
| | | GTQAMDEADYYCQAWDSSTWVFGGG | |
| | | TKLTVLGQPKAAPSVTLFPPSSEEL | |
| | | QANKATLVCLISDFYPGAVTVAWKA | |
| | | DSSPVKAGVETTTPSKQSNNKYAAS | |
| | | S | |
| | LC | SYELTQPPSVSVSPGQTASITCSGD | 364 |
| | variable | KLGDRYVCWYQQKPGQSPVLVIYQD | |
| | | TKRPSGIPERFSGSNSGNTATLTIS | |
| | | GTQAMDEADYYCQAWDSSTWVFGGG | |
| | | TKLTVL | |
| | LCDR1 | SGDKLGDRYVC | 365 |
| | LCDR2 | QDTKRPS | 366 |
| | LCDR3 | QAWDSSTWV | 367 |
| | LFR1 | SYELTQPPSVSVSPGQTASITC | 368 |
| | LFR2 | WYQQKPGQSPVLVIY | 369 |
| | LFR3 | GIPERFSGSNSGNTATLTISGTQAM | 370 |
| | | DEADYYC | |
| | LFR4 | FGGGTKLTVL | 69 |
| S24-1339 | HC | EVQLVESGGGLVQPGGSLRLSCAAS | 371 |
| | | GFTVSSNYMSWVRQAPGKGLEWVSD | |
| | | IYSGGSTYYADSVKGRFTISRHNSK | |
| | | NTLYLQMNSLRAEDTAVYYCARDRR | |
| | | GYSYGLHHGMDVWGQGTTVTVSSAS | |
| | | TKGPSVFPLAPSSKSTSGGTAALGC | |
| | | LVKDYFPEPVTVSWNSGALTSGVHT | |
| | | FPAVLQSSG | |
| | HC | EVQLVESGGGLVQPGGSLRLSCAAS | 372 |
| | variable | GFTVSSNYMSWVRQAPGKGLEWVSD | |
| | | IYSGGSTYYADSVKGRFTISRHNSK | |
| | | NTLYLQMNSLRAEDTAVYYCARDRR | |
| | | GYSYGLHHGMDVWGQGTTVTVSS | |
| | HCDR1 | SNYMS | 358 |
| | HCDR2 | DIYSGGSTYYADSVKG | 373 |
| | HCDR3 | DRRGYSYGLHHGMDV | 374 |
| | HFR1 | EVQLVESGGGLVQPGGSLRLSCAAS | 361 |
| | | GFTVS | |
| | HFR2 | WVRQAPGKGLEWVS | 130 |
| | HFR3 | RFTISRHNSKNTLYLQMNSLRAEDT | 375 |
| | | AVYYCAR | |
| | HFR4 | WGQGTTVTVSS | 147 |
| | LC | EIVLTQSPGTLSLSPGERATLSCRA | 376 |
| | | SQSVSSSYLAWYQQKPDQAPRLLIY | |
| | | GASSRATGIPDRFSGSGSGTDFTLT | |
| | | ISRLEPEDFAVYYCQQYGSSPNTFG | |
| | | QGTKLEIKRTVAAPSVFIFPPSDEQ | |
| | | LKSGTASVVCLLNNFYPREAKVQWK | |
| | | VDN | |
| | LC | EIVLTQSPGTLSLSPGERATLSCRA | 377 |
| | variable | SQSVSSSYLAWYQQKPDQAPRLLIY | |
| | | GASSRATGIPDRFSGSGSGTDFTLT | |
| | | ISRLEPEDFAVYYCQQYGSSPNTFG | |
| | | QGTKLEIK | |
| | LCDR1 | RASQSVSSSYLA | 338 |
| | LCDR2 | GASSRAT | 135 |
| | LCDR3 | QQYGSSPNT | 378 |
| | LFR1 | EIVLTQSPGTLSLSPGERATLSC | 50 |
| | LFR2 | WYQQKPDQAPRLLIY | 379 |
| | LFR3 | GIPDRFSGSGSGTDFTLTISRLEPE | 196 |
| | | DFAVYYC | |
| | LFR4 | FGQGTKLEIK | 380 |
| S24-1345 | HC | QLQLQESGPGLVKPSETLSLTCTVS | 381 |
| | | GGSISSSSYYWGWIRQPPGKGLEWI | |
| | | GSIYYSGSTYYNPSLKSRVTISVDT | |
| | | SKNQFSLKLSSVTAADTAVYYCARR | |
| | | IRRPTSEVVITYVFDYWGQGTLVTV | |
| | | SSAPTKAPDVFPIISGCRHPKDNSP | |
| | | VVLACLITGYH | |
| | HC | QLQLQESGPGLVKPSETLSLTCTVS | 382 |
| | variable | GGSISSSSYYWGWIRQPPGKGLEWI | |
| | | GSIYYSGSTYYNPSLKSRVTISVDT | |
| | | SKNQFSLKLSSVTAADTAVYYCARR | |
| | | IRRPTSEVVITYVFDYWGQGTLVTV | |
| | | SS | |
| | HCDR1 | SSSYYWG | 242 |
| | HCDR2 | SIYYSGSTYYNPSLKS | 243 |
| | HCDR3 | RIRRPTSEVVITYVFDY | 383 |
| | HFR1 | QLQLQESGPGLVKPSETLSLTCTVS | 245 |
| | | GGSIS | |
| | HFR2 | WIRQPPGKGLEWIG | 7 |
| | HFR3 | RVTISVDTSKNQFSLKLSSVTAADT | 115 |
| | | AVYYCAR | |
| | HFR4 | WGQGTLVTVSS | 60 |
| | LC | AIQLTQSPSSLSASVGDRVTITCRA | 384 |
| | | SQGISSALAWYQQKPGKAPKLLIYD | |
| | | ASSLESGVPSRFSGSGSGTDFTLTI | |
| | | SSLQPEDFATYYCQQFNSYLTFGGG | |
| | | TKVEIKRTVAAPSVFIFPPSDEQLK | |
| | | SGTASVVCLLNNFYPREAKVQWKVD | |
| | | NALQSGNSQESVTEQDSKDSTYSLS | |
| | LC | AIQLTQSPSSLSASVGDRVTITCRA | 385 |
| | variable | SQGISSALAWYQQKPGKAPKLLIYD | |
| | | ASSLESGVPSRFSGSGSGTDFTLTI | |
| | | SSLQPEDFATYYCQQFNSYLTFGGG | |
| | | TKVEIK | |
| | LCDR1 | RASQGISSALA | 386 |
| | LCDR2 | DASSLES | 387 |
| | LCDR3 | QQFNSYLT | 388 |
| | LFR1 | AIQLTQSPSSLSASVGDRVTITC | 389 |
| | LFR2 | WYQQKPGKAPKLLIY | 252 |
| | LFR3 | GVPSRFSGSGSGTDFTLTISSLQPE | 253 |
| | | DFATYYC | |
| | LFR4 | FGGGTKVEIK | 85 |
| S24-1378 | HC | EVQLVESGGGLVQPGGSLRLSCAAS | 390 |
| | | GFTVSSNYMSWVRQAPGKGLEWVSV | |
| | | IYSGGSTYYADSVKGRFTISRHNSK | |
| | | NTLYLQMNSLRAEDTAVYYCAREGY | |
| | | CTNGVCYRHAFDIWGQGTMVTVSSG | |
| | | SASAPTLFPLVSCENSPSDTSSV | |
| | HC | EVQLVESGGGLVQPGGSLRLSCAAS | 391 |
| | variable | GFTVSSNYMSWVRQAPGKGLEWVSV | |
| | | IYSGGSTYYADSVKGRFTISRHNSK | |
| | | NTLYLQMNSLRAEDTAVYYCAREGY | |
| | | CTNGVCYRHAFDIWGQGTMVTVSS | |
| | HCDR1 | SNYMS | 358 |
| | HCDR2 | VIYSGGSTYYADSVKG | 392 |
| | HCDR3 | EGYCTNGVCYRHAFDI | 393 |
| | HFR1 | EVQLVESGGGLVQPGGSLRLSCAAS | 361 |
| | | GFTVS | |
| | HFR2 | WVRQAPGKGLEWVS | 130 |
| | HFR3 | RFTISRHNSKNTLYLQMNSLRAEDT | 375 |
| | | AVYYCAR | |
| | HFR4 | WGQGTMVTVSS | 44 |
| | LC | QTVVTQEPSFSVSPGGTVTLTCGLS | 394 |
| | | SGSVSTSYYPSWYQQTPGQAPRTLI | |
| | | YSTNTRSSGVPDRFSGSILGNKAAL | |
| | | TITGAQADDESDYYCVLYMGSGISV | |
| | | FGGGTKLTVLGQPKAAPSVTLFPPS | |
| | | SEELQANKATLVCLISDFYPGAVTV | |
| | | AWKADSSPVKAGVETTTPSKQSNNK | |
| | | YAASS | |
| | LC | QTVVTQEPSFSVSPGGTVTLTCGLS | 395 |
| | variable | SGSVSTSYYPSWYQQTPGQAPRTLI | |
| | | YSTNTRSSGVPDRFSGSILGNKAAL | |
| | | TITGAQADDESDYYCVLYMGSGISV | |
| | | FGGGTKLTVL | |
| | LCDR1 | GLSSGSVSTSYYPS | 276 |
| | LCDR2 | STNTRSS | 396 |
| | LCDR3 | VLYMGSGISV | 397 |
| | LFR1 | QTVVTQEPSFSVSPGGTVTLTC | 279 |
| | LFR2 | WYQQTPGQAPRTLIY | 280 |
| | LFR3 | GVPDRFSGSILGNKAALTITGAQAD | 281 |
| | | DESDYYC | |
| | LFR4 | FGGGTKLTVL | 69 |
| S24-1379 | HC | QVQLQESGPGLVKPSETLSLTCTVS | 398 |
| | | GGSISSYYWSWIRQPPGKGLEWIGY | |
| | | IYYSGSTNYNPSLKSRVTISVDTSK | |
| | | NQFSLKLSSVTAADTAVYYCARDYY | |
| | | QLPMDVWGQGTTVTVSSASTKGPSV | |
| | | FPLAPSSKSTSGGTAALGCLVKDYF | |
| | | PEPVTVSWNSGALTSGVHTFPAVLQ | |
| | | SSG | |
| | HC | QVQLQESGPGLVKPSETLSLTCTVS | 399 |
| | variable | GGSISSYYWSWIRQPPGKGLEWIGY | |
| | | IYYSGSTNYNPSLKSRVTISVDTSK | |
| | | NQFSLKLSSVTAADTAVYYCARDYY | |
| | | QLPMDVWGQGTTVTVSS | |
| | HCDR1 | SYYWS | 56 |
| | HCDR2 | YIYYSGSTNYNPSLKS | 4 |
| | HCDR3 | DYYQLPMDV | 400 |
| | HFR1 | QVQLQESGPGLVKPSETLSLTCTVS | 24 |
| | | GGSIS | |
| | HFR2 | WIRQPPGKGLEWIG | 7 |
| | HFR3 | RVTISVDTSKNQFSLKLSSVTAADT | 115 |
| | | AVYYCAR | |
| | HFR4 | WGQGTTVTVSS | 147 |
| | LC | QSVLTQPPSASGTPGQRVTISCSGS | 401 |
| | | SSNIGSNYVYWYQQLPGTAPKLLIY | |
| | | RNNQRPSGVPDRFSGSKSGTSASLA | |
| | | ISGLRSEDEADYYCAAWDDSLSGRV | |
| | | FGGGT | |
| | | KLTVLGQPKAAPSVTLFPPSSEELQ | |
| | | ANKATLVCLISDFYPGAVTVAWKAD | |
| | | SSPVKAGVETTTPSKQSNNKYAASS | |
| | LC | QSVLTQPPSASGTPGQRVTISCSGS | 402 |
| | variable | SSNIGSNYVYWYQQLPGTAPKLLIY | |
| | | RNNQRPSGVPDRFSGSKSGTSASLA | |
| | | ISGLRSEDEADYYCAAWDDSLSGRV | |
| | | FGGGTKLTVL | |
| | LCDR1 | SGSSSNIGSNYVY | 403 |
| | LCDR2 | RNNQRPS | 404 |
| | LCDR3 | AAWDDSLSGRV | 405 |
| | LFR1 | QSVLTQPPSASGTPGQRVTISC | 121 |
| | LFR2 | WYQQLPGTAPKLLIY | 122 |
| | LFR3 | GVPDRFSGSKSGTSASLAISGLRSE | 406 |
| | | DEADYYC | |
| | LFR4 | FGGGTKLTVL | 69 |
| S24-1384 | HC | EVQLVESGGGLVQPGGSLRLSCAVS | 407 |
| | | GFTFSSYSMNWVRQAPGKGLEWVSY | |
| | | ISSSSSIIYYADSVKGRFTISRDNA | |
| | | KNSLYLQMNSLRAEDTAVYYCARDF | |
| | | LDYSRSYSYGMDVWGQGTTVTVSSA | |
| | | STKGPSVFPLAPSSKSTSGGTAALG | |
| | | CLVKDYFPEPVTVSWNSGALTSGVH | |
| | | TFPAVLQSSG | |
| | HC | EVQLVESGGGLVQPGGSLRLSCAVS | 408 |
| | variable | GFTFSSYSMNWVRQAPGKGLEWVSY | |
| | | ISSSSSIIYYADSVKGRFTISRDNA | |
| | | KNSLYLQMNSLRAEDTAVYYCARDF | |
| | | LDYSRSYSYGMDVWGQGTTVTVSS | |
| | HCDR1 | SYSMN | 126 |
| | HCDR2 | YISSSSSIIYYADSVKG | 409 |
| | HCDR3 | DFLDYSRSYSYGMDV | 410 |
| | HFR1 | EVQLVESGGGLVQPGGSLRLSCAVS | 411 |
| | | GFTFS | |
| | HFR2 | WVRQAPGKGLEWVS | 130 |
| | HFR3 | RFTISRDNAKNSLYLQMNSLRAEDT | 273 |
| | | AVYYCAR | |
| | HFR4 | WGQGTTVTVSS | 147 |
| | LC | SYVLTQPPSVSVAPGQTARITCGGD | 412 |
| | | NIGSKNVHWYQQKPGQAPVLVVFDD | |
| | | SDRPSGIPERFSGSNSGNTATLTIS | |
| | | RVEAGDEADYYCQVWDSSSDHYVVF | |
| | | GGGTKLTVLGQPKAAPSVTLFPPSS | |
| | | EELQANKATLVCLISDFYPGAVTVA | |
| | | WKADSSPVKAGVETTTPSKQSNNKY | |
| | | AASSY | |
| | LC | SYVLTQPPSVSVAPGQTARITCGGD | 413 |
| | variable | NIGSKNVHWYQQKPGQAPVLVVFDD | |
| | | SDRPSGIPERFSGSNSGNTATLTIS | |
| | | RVEAGDEADYYCQVWDSSSDHYVVF | |
| | | GGGTKLTVL | |
| | LCDR1 | GGDNIGSKNVH | 414 |
| | LCDR2 | DDSDRPS | 13 |
| | LCDR3 | QVWDSSSDHYVV | 415 |
| | LFR1 | SYVLTQPPSVSVAPGQTARITC | 15 |
| | LFR2 | WYQQKPGQAPVLVVF | 416 |
| | LFR3 | GIPERFSGSNSGNTATLTISRVEAG | 17 |
| | | DEADYYC | |
| | LFR4 | FGGGTKLTVL | 69 |
| S24-1476 | HC | EVQLVESGGGLVQPGRSLRLSCTAS | 417 |
| | | GFTFGDYAMSWFRQAPGKGLEWVGF | |
| | | IRSKAYGGTTQYAASVKGRFTISRD | |
| | | DSKSIAYLQMNSLKTEDTAVYYCTR | |
| | | VRYCTNGVCYGYHFDYWGQGTVVTV | |
| | | SSAST | |
| | HC | EVQLVESGGGLVQPGRSLRLSCTAS | 418 |
| | variable | GFTFGDYAMSWFRQAPGKGLEWVGF | |
| | | IRSKAYGGTTQYAASVKGRFTISRD | |
| | | DSKSIAYLQMNSLKTEDTAVYYCTR | |
| | | VRYCTNGVCYGYHFDYWGQGTVVTV | |
| | | SS | |
| | HCDR1 | DYAMS | 199 |
| | HCDR2 | FIRSKAYGGTTQYAASVKG | 419 |
| | HCDR3 | VRYCTNGVCYGYHFDY | 420 |
| | HFR1 | EVQLVESGGGLVQPGRSLRLSCTAS | 202 |
| | | GFTFG | |
| | HFR2 | WFRQAPGKGLEWVG | 203 |
| | HFR3 | RFTISRDDSKSIAYLQMNSLKTEDT | 259 |
| | | AVYYCTR | |
| | HFR4 | WGQGTVVTVSS | 421 |
| | LC | EIVMTQSPATLSVSPGERATLSCRA | 422 |
| | | SQSVSSNLAWYQQKPGQAPRLLIYG | |
| | | ASTRATGIPARFSGSGSGTEFTLTI | |
| | | SSLQSEDFAVYYCQQYNNWWTFGQG | |
| | | TKVEIKRTVAAPSVFIFPPSDEQLK | |
| | | SGTASVVCLLNNFYPREAKVQWKVD | |
| | | N | |
| | LC | EIVMTQSPATLSVSPGERATLSCRA | 423 |
| | variable | SQSVSSNLAWYQQKPGQAPRLLIYG | |
| | | ASTRATGIPARFSGSGSGTEFTLTI | |
| | | SSLQSEDFAVYYCQQYNNWWTFGQG | |
| | | TKVEIK | |
| | LCDR1 | RASQSVSSNLA | 207 |
| | LCDR2 | GASTRAT | 208 |
| | LCDR3 | QQYNNWWT | 424 |
| | LFR1 | EIVMTQSPATLSVSPGERATLSC | 210 |
| | LFR2 | WYQQKPGQAPRLLIY | 182 |
| | LFR3 | GIPARFSGSGSGTEFTLTISSLQSE | 211 |
| | | DFAVYYC | |
| | LFR4 | FGQGTKVEIK | 53 |
| S24-1564 | HC | QVQLQESGPGLVKPSETLSLTCTVS | 425 |
| | | GGSISSYYWSWIRQPPGKGLEWIGY | |
| | | VYYSGNTKYNPSLKSRVTISVDTSK | |
| | | NQFSLKLGSVTAADTAVYYCARHSR | |
| | | IEVAGTLDFDYWGQGTLVTVSSAST | |
| | | KGPSVFPLAPSSKSTSGGTAALGCL | |
| | | VKDYFPEPVTVSWNSGALTSGVHTF | |
| | | PAVLQSSG | |
| | HC | QVQLQESGPGLVKPSETLSLTCTVS | 426 |
| | variable | GGSISSYYWSWIRQPPGKGLEWIGY | |
| | | VYYSGNTKYNPSLKSRVTISVDTSK | |
| | | NQFSLKLGSVTAADTAVYYCARHSR | |
| | | IEVAGTLDFDYWGQGTLVTVSS | |
| | HCDR1 | SYYWS | 56 |
| | HCDR2 | YVYYSGNTKYNPSLKS | 427 |
| | HCDR3 | HSRIEVAGTLDFDY | 428 |
| | HFR1 | QVQLQESGPGLVKPSETLSLTCTVS | 24 |
| | | GGSIS | |
| | HFR2 | WIRQPPGKGLEWIG | 7 |
| | HFR3 | RVTISVDTSKNQFSLKLGSVTAADT | 429 |
| | | AVYYCAR | |
| | HFR4 | WGQGTLVTVSS | 60 |
| | LC | DIQMTQSPSSLSASVGDRVTITCRA | 430 |
| | | SQSIRSYLNWYQQKRGKAPKLLIYA | |
| | | ASSLQSGVPSRFSGSGSGTDFTLTI | |
| | | SSLQPEDFATYYCQQSYSTPPTFGQ | |
| | | GTKVEIKRTVAAPSVFIFPPSDEQL | |
| | | KSGTASVVCLLNNFYPREAKVQWKV | |
| | | DN | |
| | LC | DIQMTQSPSSLSASVGDRVTITCRA | 431 |
| | variable | SQSIRSYLNWYQQKRGKAPKLLIYA | |
| | | ASSLQSGVPSRFSGSGSGTDFTLTI | |
| | | SSLQPEDFATYYCQQSYSTPPTFGQ | |
| | | GTKVEIK | |
| | LCDR1 | RASQSIRSYLN | 432 |
| | LCDR2 | AASSLQS | 249 |
| | LCDR3 | QQSYSTPPT | 433 |
| | LFR1 | DIQMTQSPSSLSASVGDRVTITC | 251 |
| | LFR2 | WYQQKRGKAPKLLIY | 434 |
| | LFR3 | GVPSRFSGSGSGTDFTLTISSLQPE | 253 |
| | | DFATYYC | |
| | LFR4 | FGQGTKVEIK | 53 |
| S24-1636 | HC | QVQLVESGGGVVQPGRSLRLSCAAS | 435 |
| | | GFTFSNYGMHWVRQAPGKGLEWVAV | |
| | | IWYDGSNKYYADSVKGRFTISRDNS | |
| | | KNTLYLQMNSLRAEDTAVYYCARGD | |
| | | CTNGVCHPLLIYYDSSGLDYWGQGT | |
| | | LVTVSSASTKGPSVFPLAPSSKSTS | |
| | | GGTAALGCLVKDYFPEPVTVSWNSG | |
| | | ALTSGVHTFPAVLQSSG | |
| | HC | QVQLVESGGGVVQPGRSLRLSCAAS | 436 |
| | variable | GFTFSNYGMHWVRQAPGKGLEWVAV | |
| | | IWYDGSNKYYADSVKGRFTISRDNS | |
| | | KNTLYLQMNSLRAEDTAVYYCARGD | |
| | | CTNGVCHPLLIYYDSSGLDYWGQGT | |
| | | LVTVSS | |
| | HCDR1 | NYGMH | 437 |
| | HCDR2 | VIWYDGSNKYYADSVKG | 142 |
| | HCDR3 | GDCTNGVCHPLLIYYDSSGLDY | 438 |
| | HFR1 | QVQLVESGGGVVQPGRSLRLSCAAS | 144 |
| | | GFTFS | |
| | HFR2 | WVRQAPGKGLEWVA | 145 |
| | HFR3 | RFTISRDNSKNTLYLQMNSLRAEDT | 146 |
| | | AVYYCAR | |
| | HFR4 | WGQGTLVTVSS | 60 |
| | LC | EIVLTQSPATLSLSPGERATLSCRA | 439 |
| | | SQSVSSYLAWYQQKPGQAPRLLIYD | |
| | | ASNRATGIPARFSGSGSGTDFTLTI | |
| | | SSLEPEDFAVYYCQQRSNWPPITFG | |
| | | PGTKVDIKRTVAAPSVFIFPPSDEQ | |
| | | LKSGTASVVCLLNNFYPREAKVQWK | |
| | | VDNALQSGNSQESVTEQDSKDSTYS | |
| | | L | |
| | LC | EIVLTQSPATLSLSPGERATLSCRA | 440 |
| | variable | SQSVSSYLAWYQQKPGQAPRLLIYD | |
| | | ASNRATGIPARFSGSGSGTDFTLTI | |
| | | SSLEPEDFAVYYCQQRSNWPPITFG | |
| | | PGTKVDIK | |
| | LCDR1 | RASQSVSSYLA | 178 |
| | LCDR2 | DASNRAT | 441 |
| | LCDR3 | QQRSNWPPIT | 442 |
| | LFR1 | EIVLTQSPATLSLSPGERATLSC | 181 |
| | LFR2 | WYQQKPGQAPRLLIY | 182 |
| | LFR3 | GIPARFSGSGSGTDFTLTISSLEPE | 183 |
| | | DFAVYYC | |
| | LFR4 | FGPGTKVDIK | 443 |
| S24-1002 | HC | QVQLVESGGGVVQPGRSLRLSCAAS | 444 |
| | | GFTFTSYAMHWVRQAPGKGLEWVAV | |
| | | ISYDGGSKYYADSVKGRFTISRDNS | |
| | | KNTLYLQMNSLRAEDTAVYYCARTT | |
| | | PGITAAGTGTLGRYYYYGMDVWGQG | |
| | | TTVTVSSGSASAPTLFPLVSCENSP | |
| | | SDTSSV | |
| | HC | QVQLVESGGGVVQPGRSLRLSCAAS | 445 |
| | variable | GFTFTSYAMHWVRQAPGKGLEWVAV | |
| | | ISYDGGSKYYADSVKGRFTISRDNS | |
| | | KNTLYLQMNSLRAEDTAVYYCARTT | |
| | | PGITAAGTGTLGRYYYYGMDVWGQG | |
| | | TTVTVSS | |
| | HCDR1 | SYAMH | 446 |
| | HCDR2 | VISYDGGSKYYADSVKG | 447 |
| | HCDR3 | TTPGITAAGTGTLGRYYYYGMDV | 448 |
| | HFR1 | QVQLVESGGGVVQPGRSLRLSCAAS | 449 |
| | | GFTFT | |
| | HFR2 | WVRQAPGKGLEWVA | 145 |
| | HFR3 | RFTISRDNSKNTLYLQMNSLRAEDT | 146 |
| | | AVYYCAR | |
| | HFR4 | WGQGTTVTVSS | 147 |
| | LC | AIQLTQSPSSLSASVGDRVTITCRA | 450 |
| | | SQGISSALAWYQQTPGKAPKLLIYD | |
| | | ASSLESGVPSRFSGSGSGTDFSLTI | |
| | | GSLQPEDFASYYCQQFNSYPLTFGG | |
| | | GTKVEIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | AIQLTQSPSSLSASVGDRVTITCRA | 451 |
| | variable | SQGISSALAWYQQTPGKAPKLLIYD | |
| | | ASSLESGVPSRFSGSGSGTDFSLTI | |
| | | GSLQPEDFASYYCQQFNSYPLTFGG | |
| | | GTKVEIK | |
| | LCDR1 | RASQGISSALA | 386 |
| | LCDR2 | DASSLES | 387 |
| | LCDR3 | QQFNSYPLT | 452 |
| | LFR1 | AIQLTQSPSSLSASVGDRVTITC | 389 |
| | LFR2 | WYQQTPGKAPKLLIY | 453 |
| | LFR3 | GVPSRFSGSGSGTDFSLTIGSLQPE | 454 |
| | | DFASYYC | |
| | LFR4 | FGGGTKVEIK | 85 |
| S24-1301 | HC | QVQLVQSGAEVKKPGASVKVSCKVS | 455 |
| | | GYTLIELSMHWVRQAPGKGLEWMGG | |
| | | FDPEDGETIYAQKFQGRVTMTEDTS | |
| | | TDTAYMALSSLTSEDTAVYYCATAY | |
| | | AYYYASGGYYTLDYWGQGTLVTVSS | |
| | | ASTKGPSVFPLAPSSKSTSGGTAAL | |
| | | GCLVKDYFPEPVTVSWNSGALTSGV | |
| | | HTFPAVLQSSG | |
| | HC | QVQLVQSGAEVKKPGASVKVSCKVS | 456 |
| | variable | GYTLIELSMHWVRQAPGKGLEWMGG | |
| | | FDPEDGETIYAQKFQGRVTMTEDTS | |
| | | TDTAYMALSSLTSEDTAVYYCATAY | |
| | | AYYYASGGYYTLDYWGQGTLVTVSS | |
| | HCDR1 | ELSMH | 457 |
| | HCDR2 | GFDPEDGETIYAQKFQG | 458 |
| | HCDR3 | AYAYYYASGGYYTLDY | 459 |
| | HFR1 | QVQLVQSGAEVKKPGASVKVSCKVS | 460 |
| | | GYTLI | |
| | HFR2 | WVRQAPGKGLEWMG | 42 |
| | HFR3 | RVTMTEDTSTDTAYMALSSLTSEDT | 461 |
| | | AVYYCAT | |
| | HFR4 | WGQGTLVTVSS | 60 |
| | LC | QAGLTQPPSVSKGLRQTATLTCTGS | 462 |
| | | SNNVGNQGAAWLQQHQGHPPKLLSY | |
| | | RNNNRPSGISERFSASRSGNTASLT | |
| | | ITGLQPEDEADYYCSAWDSSLSNWV | |
| | | FGGGTKLTVLGQPKAAPSVTLFPPS | |
| | | SEELQANKATLVCLISDFYPGAVTV | |
| | | AWKADSSPVKAGVETTTPSKQSNNK | |
| | | YAASS | |
| | LC | QAGLTQPPSVSKGLRQTATLTCTGS | 463 |
| | variable | SNNVGNQGAAWLQQHQGHPPKLLSY | |
| | | RNNNRPSGISERFSASRSGNTASLT | |
| | | ITGLQPEDEADYYCSAWDSSLSNWV | |
| | | FGGGTKLTVL | |
| | LCDR1 | TGSSNNVGNQGAA | 464 |
| | LCDR2 | RNNNRPS | 465 |
| | LCDR3 | SAWDSSLSNWV | 466 |
| | LFR1 | QAGLTQPPSVSKGLRQTATLTC | 467 |
| | LFR2 | WLQQHQGHPPKLLSY | 468 |
| | LFR3 | GISERFSASRSGNTASLTITGLQPE | 469 |
| | | DEADYYC | |
| | LFR4 | FGGGTKLTVL | 69 |
| S24-223 | HC | QITLKESGPTLVKPTQTLTLTCTFS | 470 |
| | | GFSLNTSGVGVGWIRQPPGKALEWL | |
| | | ALIYWDDDKRYSPSLKSRLTITKDT | |
| | | SKNQVVLTMTNMDPVDTATYYCAHH | |
| | | TIVPIFDYWGQGTLVTVSSGSASAP | |
| | | TLFPLVSCENSPSDTSSV | |
| | HC | QITLKESGPTLVKPTQTLTLTCTFS | 471 |
| | variable | GFSLNTSGVGVGWIRQPPGKALEWL | |
| | | ALIYWDDDKRYSPSLKSRLTITKDT | |
| | | SKNQVVLTMTNMDPVDTATYYCAHH | |
| | | TIVPIFDYWGQGTLVTVSS | |
| | HCDR1 | TSGVGVG | 472 |
| | HCDR2 | LIYWDDDKRYSPSLKS | 473 |
| | HCDR3 | HTIVPIFDY | 474 |
| | HFR1 | QITLKESGPTLVKPTQTLTLTCTFS | 475 |
| | | GFSLN | |
| | HFR2 | WIRQPPGKALEWLA | 476 |
| | HFR3 | RLTITKDTSKNQVVLTMTNMDPVDT | 477 |
| | | ATYYCAH | |
| | HFR4 | WGQGTLVTVSS | 60 |
| | LC | QSALTQPASVSGSPGQSITISCTGT | 478 |
| | | SSDVGGYNYVSWYQQHPGKAPKLMI | |
| | | YDVSNRPSGVSNRFSGSKSGNTASL | |
| | | TISGLQAEDEADYYCNSYTSSSTLV | |
| | | VFGGGTKLTVLGQPKAAPSVTLFPP | |
| | | SSEELQANKATLVCLISDFYPGAVT | |
| | | VAWKADSSPVKAGVETTTPSKQSNN | |
| | | KYAASSYLSLT | |
| | LC | QSALTQPASVSGSPGQSITISCTGT | 479 |
| | variable | SSDVGGYNYVSWYQQHPGKAPKLMI | |
| | | YDVSNRPSGVSNRFSGSKSGNTASL | |
| | | TISGLQAEDEADYYCNSYTSSSTLV | |
| | | VFGGGTKLTVL | |
| | LCDR1 | TGTSSDVGGYNYVS | 63 |
| | LCDR2 | DVSNRPS | 64 |
| | LCDR3 | NSYTSSSTLVV | 480 |
| | LFR1 | QSALTQPASVSGSPGQSITISC | 66 |
| | LFR2 | WYQQHPGKAPKLMIY | 67 |
| | LFR3 | GVSNRFSGSKSGNTASLTISGLQAE | 68 |
| | | DEADYYC | |
| | LFR4 | FGGGTKLTVL | 69 |
| S24-461 | HC | QVQLQESGPGLVKPSETLSLTCTVS | 481 |
| | | GGSISSYYWSWIRQPPGKGLEWIGN | |
| | | IYNSGSTNYNPSLKSRLTISVDTSK | |
| | | NHFSLKLSSVTAADTAVYYCARGGL | |
| | | EHDGDYVYYYGMDVWGQGTTITVSS | |
| | | ASTKGPSVFPLAPSSKSTSGGTAAL | |
| | | GCLVKDYFPEPVTVSWNSGALTSGV | |
| | | HTFPAVLQSSG | |
| | HC | QVQLQESGPGLVKPSETLSLTCTVS | 482 |
| | variable | GGSISSYYWSWIRQPPGKGLEWIGN | |
| | | IYNSGSTNYNPSLKSRLTISVDTSK | |
| | | NHFSLKLSSVTAADTAVYYCARGGL | |
| | | EHDGDYVYYYGMDVWGQGTTITVSS | |
| | HCDR1 | SYYWS | 56 |
| | HCDR2 | NIYNSGSTNYNPSLKS | 483 |
| | HCDR3 | GGLEHDGDYVYYYGMDV | 484 |
| | HFR1 | QVQLQESGPGLVKPSETLSLTCTVS | 24 |
| | | GGSIS | |
| | HFR2 | WIRQPPGKGLEWIG | 7 |
| | HFR3 | RLTISVDTSKNHFSLKLSSVTAADT | 485 |
| | | AVYYCAR | |
| | HFR4 | WGQGTTITVSS | 486 |
| | LC | SYELTQPPSVSVSLGQMARITCSGE | 487 |
| | | ALPKKYAYWYQQKPGQFPILVIYKD | |
| | | SERPSGIPERFSGSSSGTIVTLTIS | |
| | | GVQAEDEADYYCLSEDSSGTWVFGG | |
| | | GTKLTVLGQPKAAPSVTLFPPSSEE | |
| | | LQANKATLVCLISDFYPGAVTVAWK | |
| | | ADSSPVKAGVETTTPSKQSNNKYAA | |
| | | SS | |
| | LC | SYELTQPPSVSVSLGQMARITCSGE | 488 |
| | variable | ALPKKYAYWYQQKPGQFPILVIYKD | |
| | | SERPSGIPERFSGSSSGTIVTLTIS | |
| | | GVQAEDEADYYCLSEDSSGTWVFGG | |
| | | GTKLTVL | |
| | LCDR1 | SGEALPKKYAY | 489 |
| | LCDR2 | KDSERPS | 490 |
| | LCDR3 | LSEDSSGTWV | 491 |
| | LFR1 | SYELTQPPSVSVSLGQMARITC | 492 |
| | LFR2 | WYQQKPGQFPILVIY | 493 |
| | LFR3 | GIPERFSGSSSGTIVTLTISGVQAE | 494 |
| | | DEADYYC | |
| | LFR4 | FGGGTKLTVL | 69 |
| S24-511 | HC | QVQLVESGGGVVQPGRSLRLSCAAS | 495 |
| | | GFTFSSYGMHWVRQAPGKGLEWVAV | |
| | | ISYDGSNKYYADSVKGRFTISRDNS | |
| | | KNTLYLQMNSLRAEDTAVYYCAKYT | |
| | | STVTTNYYYGMDVWGQGTTVTVSSA | |
| | | PTKAPDVFPIISGCRHPKDNSPVVL | |
| | | ACLITGYH | |
| | HC | QVQLVESGGGVVQPGRSLRLSCAAS | 496 |
| | variable | GFTFSSYGMHWVRQAPGKGLEWVAV | |
| | | ISYDGSNKYYADSVKGRFTISRDNS | |
| | | KNTLYLQMNSLRAEDTAVYYCAKYT | |
| | | STVTTNYYYGMDVWGQGTTVTVSS | |
| | HCDR1 | SYGMH | 141 |
| | HCDR2 | VISYDGSNKYYADSVKG | 497 |
| | HCDR3 | YTSTVTTNYYYGMDV | 498 |
| | HFR1 | QVQLVESGGGVVQPGRSLRLSCAAS | 144 |
| | | GFTFS | |
| | HFR2 | WVRQAPGKGLEWVA | 145 |
| | HFR3 | RFTISRDNSKNTLYLQMNSLRAEDT | 499 |
| | | AVYYCAK | |
| | HFR4 | WGQGTTVTVSS | 147 |
| | LC | SYELTQPPSVSVSPGQTASITCSGD | 500 |
| | | KLGDKYACWYQQKPGQSPVLVIYQD | |
| | | SKRPSGIPERFSGSNSGNTATLTIS | |
| | | GTQAMDEADYYCQAWDSSTVVFGGG | |
| | | TKLTVLGQPKAAPSVTLFPPSSEEL | |
| | | QANKATLVCLISDFYPGAVTVAWKA | |
| | | DSSPVKAGVETTTPSKQSNNKYAAS | |
| | | SY | |
| | LC | SYELTQPPSVSVSPGQTASITCSGD | 501 |
| | variable | KLGDKYACWYQQKPGQSPVLVIYQD | |
| | | SKRPSGIPERFSGSNSGNTATLTIS | |
| | | GTQAMDEADYYCQAWDSSTVVFGGG | |
| | | TKLTVL | |
| | LCDR1 | SGDKLGDKYAC | 502 |
| | LCDR2 | QDSKRPS | 503 |
| | LCDR3 | QAWDSSTVV | 504 |
| | LFR1 | SYELTQPPSVSVSPGQTASITC | 368 |
| | LFR2 | WYQQKPGQSPVLVIY | 369 |
| | LFR3 | GIPERFSGSNSGNTATLTISGTQAM | 370 |
| | | DEADYYC | |
| | LFR4 | FGGGTKLTVL | 69 |
| S24-788 | HC | QVQLVESGGGVVQPGRSLRLSCAAS | 505 |
| | | GFTFSSYGMHWVRQAPGKGLEWVAV | |
| | | IWYDGSNKYYADSVKGRFTISRDNS | |
| | | KNTLYLQMNSLRAEDTAVYYCARGR | |
| | | SPGGGHYYGMDVWGQGTTVTVSSGS | |
| | | ASAPTLFPLVSCENSPSDTSSV | |
| | HC | QVQLVESGGGVVQPGRSLRLSCAAS | 506 |
| | variable | GFTFSSYGMHWVRQAPGKGLEWVAV | |
| | | IWYDGSNKYYADSVKGRFTISRDNS | |
| | | KNTLYLQMNSLRAEDTAVYYCARGR | |
| | | SPGGGHYYGMDVWGQGTTVTVSS | |
| | HCDR1 | SYGMH | 141 |
| | HCDR2 | VIWYDGSNKYYADSVKG | 142 |
| | HCDR3 | GRSPGGGHYYGMDV | 507 |
| | HFR1 | QVQLVESGGGVVQPGRSLRLSCAAS | 144 |
| | | GFTFS | |
| | HFR2 | WVRQAPGKGLEWVA | 145 |
| | HFR3 | RFTISRDNSKNTLYLQMNSLRAEDT | 146 |
| | | AVYYCAR | |
| | HFR4 | WGQGTTVTVSS | 147 |
| | LC | SYELTQPPSVSVSPGQTASITCSGD | 508 |
| | | KLGDKYACWYQQKPGQSPVLVIYQD | |
| | | SKRPSGIPERFSGSNSGNTATLTIS | |
| | | GTQAMDEADYYCQAWDSSSVVFGGG | |
| | | TKLTVLGQPKAAPSVTLFPPSSEEL | |
| | | QANKATLVCLISDFYPGAVTVAWKA | |
| | | DSSPVKAGVETTTPSKQSNNKYAAS | |
| | | S | |
| | LC | SYELTQPPSVSVSPGQTASITCSGD | 509 |
| | variable | KLGDKYACWYQQKPGQSPVLVIYQD | |
| | | SKRPSGIPERFSGSNSGNTATLTIS | |
| | | GTQAMDEADYYCQAWDSSSVVFGGG | |
| | | TKLTVL | |
| | LCDR1 | SGDKLGDKYAC | 502 |
| | LCDR2 | QDSKRPS | 503 |
| | LCDR3 | QAWDSSSVV | 510 |
| | LFR1 | SYELTQPPSVSVSPGQTASITC | 368 |
| | LFR2 | WYQQKPGQSPVLVIY | 369 |
| | LFR3 | GIPERFSGSNSGNTATLTISGTQAM | 370 |
| | | DEADYYC | |
| | LFR4 | FGGGTKLTVL | 69 |
| S24-821 | HC | QVTLRESGPALVKPTQTLTLTCTFS | 511 |
| | | GLSLSSSGMCVSWIRQPPGKALEWL | |
| | | ARIDWDDDKYYSTSLKTRLTISKDT | |
| | | SKNQVVLTMTNMDPVDTATYYCARI | |
| | | CTMVRGLHDAFDIWGQGTMVTVSSG | |
| | | SASAPTLFPLVSCENSPSDTSSV | |
| | HC | QVTLRESGPALVKPTQTLTLTCTFS | 512 |
| | variable | GLSLSSSGMCVSWIRQPPGKALEWL | |
| | | ARIDWDDDKYYSTSLKTRLTISKDT | |
| | | SKNQVVLTMTNMDPVDTATYYCARI | |
| | | CTMVRGLHDAFDIWGQGTMVTVSS | |
| | HCDR1 | SSGMCVS | 513 |
| | HCDR2 | RIDWDDDKYYSTSLKT | 514 |
| | HCDR3 | ICTMVRGLHDAFDI | 515 |
| | HFR1 | QVTLRESGPALVKPTQTLTLTCTFS | 516 |
| | | GLSLS | |
| | HFR2 | WIRQPPGKALEWLA | 476 |
| | HFR3 | RLTISKDTSKNQVVLTMTNMDPVDT | 517 |
| | | ATYYCAR | |
| | HFR4 | WGQGTMVTVSS | 44 |
| | LC | DIQMTQSPSTLSASVGDRVTITCRA | 518 |
| | | SQSISSWLAWYQQKPGKAPKLLIYK | |
| | | ASSLESGVPSRFSGSGSGTEFTLTI | |
| | | SSLQPDDFATYYCQQYNSYSWTFGQ | |
| | | GTKVEIKRTVAAPSVFIFPPSDEQL | |
| | | KSGTASVVCLLNNFYPREAKVQWKV | |
| | | DN | |
| | LC | DIQMTQSPSTLSASVGDRVTITCRA | 519 |
| | variable | SQSISSWLAWYQQKPGKAPKLLIYK | |
| | | ASSLESGVPSRFSGSGSGTEFTLTI | |
| | | SSLQPDDFATYYCQQYNSYSWTFGQ | |
| | | GTKVEIK | |
| | LCDR1 | RASQSISSWLA | 520 |
| | LCDR2 | KASSLES | 521 |
| | LCDR3 | QQYNSYSWT | 522 |
| | LFR1 | DIQMTQSPSTLSASVGDRVTITC | 523 |
| | LFR2 | WYQQKPGKAPKLLIY | 252 |
| | LFR3 | GVPSRFSGSGSGTEFTLTISSLQPD | 524 |
| | | DFATYYC | |
| | LFR4 | FGQGTKVEIK | 53 |
| S144-67 | HC | EVQLVQSGAEVKKPGESLKISCKGS | 525 |
| | | GYSFTTYWIAWVRQMPGKGLEWVGI | |
| | | IYPDDSDTRYSPSFQGQVTISADKS | |
| | | IGTAYLQWSSLKASDTAMYYCARGQ | |
| | | YYDFWSGAGGVDVWGQGTTVTVSSA | |
| | | STKGPSVFPLAPSSKSTSGGTAALG | |
| | | CLVKDYFPEPVTVSWNSGALTSGVH | |
| | | TFPAVLQSSG | |
| | HC | EVQLVQSGAEVKKPGESLKISCKGS | 526 |
| | variable | GYSFTTYWIAWVRQMPGKGLEWVGI | |
| | | IYPDDSDTRYSPSFQGQVTISADKS | |
| | | IGTAYLQWSSLKASDTAMYYCARGQ | |
| | | YYDFWSGAGGVDVWGQGTTVTVSS | |
| | HCDR1 | TYWIA | 527 |
| | HCDR2 | IIYPDDSDTRYSPSFQG | 528 |
| | HCDR3 | GQYYDFWSGAGGVDV | 529 |
| | HFR1 | EVQLVQSGAEVKKPGESLKISCKGS | 530 |
| | | GYSFT | |
| | HFR2 | WVRQMPGKGLEWVG | 531 |
| | HFR3 | QVTISADKSIGTAYLQWSSLKASDT | 532 |
| | | AMYYCAR | |
| | HFR4 | WGQGTTVTVSS | 147 |
| | LC | QSVLTQPPSVSGAPGQRVTISCTGS | 533 |
| | | RSNIGAGYDVQWYQQVPGTAPKLLI | |
| | | SGNSNRPSGVPDRFSGSKSGTSASL | |
| | | AITGLQAEDEADYYCQSYDSSLSGL | |
| | | RVFGGGTKLTVLGQPKAAPSVTLFP | |
| | | PSSEELQANKATLVCLISDFYPGAV | |
| | | TVAWKADSSPVKAGVETTTPSKQSN | |
| | | NKYAASSYLSLTPEQWKSH | |
| | LC | QSVLTQPPSVSGAPGQRVTISCTGS | 534 |
| | variable | RSNIGAGYDVQWYQQVPGTAPKLLI | |
| | | SGNSNRPSGVPDRFSGSKSGTSASL | |
| | | AITGLQAEDEADYYCQSYDSSLSGL | |
| | | RVFGGGTKLTVL | |
| | LCDR1 | TGSRSNIGAGYDVQ | 535 |
| | LCDR2 | GNSNRPS | 350 |
| | LCDR3 | QSYDSSLSGLRV | 536 |
| | LFR1 | QSVLTQPPSVSGAPGQRVTISC | 537 |
| | LFR2 | WYQQVPGTAPKLLIS | 538 |
| | LFR3 | GVPDRFSGSKSGTSASLAITGLQAE | 539 |
| | | DEADYYC | |
| | LFR4 | FGGGTKLTVL | 69 |
| S144-69 | HC | EVQLVQSGAEVKKPGESLKISCKGS | 540 |
| | | GYSFTSYWIGWVRQMPGKGLEWMGI | |
| | | IYPGDSDTRYSPSFQGQVTISADKS | |
| | | ITTAYLQWSSLKASDTAMYYCARTQ | |
| | | TTNWFDSWGQGTLVTVSSASTKGPS | |
| | | VFPLAPSSKSTSGGTAALGCLVKDY | |
| | | FPEPVTVSWNSGALTSGVHTFPAVL | |
| | | QSSG | |
| | HC | EVQLVQSGAEVKKPGESLKISCKGS | 541 |
| | variable | GYSFTSYWIGWVRQMPGKGLEWMGI | |
| | | IYPGDSDTRYSPSFQGQVTISADKS | |
| | | ITTAYLQWSSLKASDTAMYYCARTQ | |
| | | TTNWFDSWGQGTLVTVSS | |
| | HCDR1 | SYWIG | 542 |
| | HCDR2 | IIYPGDSDTRYSPSFQG | 543 |
| | HCDR3 | TQTTNWFDS | 544 |
| | HFR1 | EVQLVQSGAEVKKPGESLKISCKGS | 530 |
| | | GYSFT | |
| | HFR2 | WVRQMPGKGLEWMG | 174 |
| | HFR3 | QVTISADKSITTAYLQWSSLKASDT | 545 |
| | | AMYYCAR | |
| | HFR4 | WGQGTLVTVSS | 60 |
| | LC | DIQMTQSPSTLSVSVGDRVTITCRA | 546 |
| | | SQSVSSWLAWYQQKPGKAPKLLIYD | |
| | | ASSLESGVPSRFSGSGSGTEFTLTI | |
| | | SSLQPDDFATYYCQQYNSFYTFGQG | |
| | | TKLEIKRTVAAPSVFIFPPSDEQLK | |
| | | SGTASVVCLLNNFYPREAKVQWKVD | |
| | | NALQSGNSQESVTEQDSKDSTYSLS | |
| | | STLTLSKADYE | |
| | LC | DIQMTQSPSTLSVSVGDRVTITCRA | 547 |
| | variable | SQSVSSWLAWYQQKPGKAPKLLIYD | |
| | | ASSLESGVPSRFSGSGSGTEFTLTI | |
| | | SSLQPDDFATYYCQQYNSFYTFGQG | |
| | | TKLEIK | |
| | LCDR1 | RASQSVSSWLA | 548 |
| | LCDR2 | DASSLES | 387 |
| | LCDR3 | QQYNSFYT | 549 |
| | LFR1 | DIQMTQSPSTLSVSVGDRVTITC | 550 |
| | LFR2 | WYQQKPGKAPKLLIY | 252 |
| | LFR3 | GVPSRFSGSGSGTEFTLTISSLQPD | 524 |
| | | DFATYYC | |
| | LFR4 | FGQGTKLEIK | 380 |
| S144-94 | HC | QVQLVESGGGVVQPGGSLRLSCAAS | 551 |
| | | GFTFSSYGMHWVRQAPGKGLEWVTF | |
| | | TRYDGSNKFYADSVKGRFSISRDNS | |
| | | KNTLYLQMNSLRAEDTAVYYCAKES | |
| | | RVAFGGAIAIYYFGMDVWGQGTTVT | |
| | | VSSASTKGPSVFPLAPCSRSTSGGT | |
| | | AALGCLVKDYFPEPVTVSWNSGALT | |
| | | SGVHTFPAVLQSSG | |
| | HC | QVQLVESGGGVVQPGGSLRLSCAAS | 552 |
| | variable | GFTFSSYGMHWVRQAPGKGLEWVTF | |
| | | TRYDGSNKFYADSVKGRFSISRDNS | |
| | | KNTLYLQMNSLRAEDTAVYYCAKES | |
| | | RVAFGGAIAIYYFGMDVWGQGTTVT | |
| | | VSS | |
| | HCDR1 | SYGMH | 141 |
| | HCDR2 | FTRYDGSNKFYADSVKG | 553 |
| | HCDR3 | ESRVAFGGAIAIYYFGMDV | 554 |
| | HFR1 | QVQLVESGGGVVQPGGSLRLSCAAS | 555 |
| | | GFTFS | |
| | HFR2 | WVRQAPGKGLEWVT | 556 |
| | HFR3 | RFSISRDNSKNTLYLQMNSLRAEDT | 557 |
| | | AVYYCAK | |
| | HFR4 | WGQGTTVTVSS | 147 |
| | LC | DIVMTQSPLSLPVTPGEPASISCRS | 558 |
| | | SQSLLHSNGYNYLDWYLQKPGQSPQ | |
| | | LLIYLGSNRASGVPDRFSGSGSGTD | |
| | | FTLKISRVEAEDVGVYYCMQALQTP | |
| | | QYTFGQGTKLEIKRTVAAPSVFIFP | |
| | | PSDEQLKSGTASVVCLLNNFYPREA | |
| | | KVQWKVDNALQSGNSQESVTEQDSK | |
| | | DSTYSLSSTLTLSKADYE | |
| | LC | DIVMTQSPLSLPVTPGEPASISCRS | 559 |
| | variable | SQSLLHSNGYNYLDWYLQKPGQSPQ | |
| | | LLIYLGSNRASGVPDRFSGSGSGTD | |
| | | FTLKISRVEAEDVGVYYCMQALQTP | |
| | | QYTFGQGTKLEIK | |
| | LCDR1 | RSSQSLLHSNGYNYLD | 262 |
| | LCDR2 | LGSNRAS | 263 |
| | LCDR3 | MQALQTPQYT | 560 |
| | LFR1 | DIVMTQSPLSLPVTPGEPASISC | 265 |
| | LFR2 | WYLQKPGQSPQLLIY | 266 |
| | LFR3 | GVPDRFSGSGSGTDFTLKISRVEAE | 267 |
| | | DVGVYYC | |
| | LFR4 | FGQGTKLEIK | 380 |
| S144-113 | HC | EVQLLESGGGLVQPGGSLRLSCAAS | 561 |
| | | GFTFSNYAMSWVRQAPGKGLEWVSA | |
| | | IRNSGSSTYYADSVKGRFTISRDNS | |
| | | KNTLYLQMNSLRAEDSAVYYCAKVG | |
| | | GTAAGHPFYDYWGQGTLVTVSSAST | |
| | | KGPSVFPLAPSSKSTSGGTAALGCL | |
| | | VKDYFPEPVTVSWNSGALTSGVHTF | |
| | | PAVLQSSGL | |
| | HC | EVQLLESGGGLVQPGGSLRLSCAAS | 562 |
| | variable | GFTFSNYAMSWVRQAPGKGLEWVSA | |
| | | IRNSGSSTYYADSVKGRFTISRDNS | |
| | | KNTLYLQMNSLRAEDSAVYYCAKVG | |
| | | GTAAGHPFYDYWGQGTLVTVSS | |
| | HCDR1 | NYAMS | 563 |
| | HCDR2 | AIRNSGSSTYYADSVKG | 564 |
| | HCDR3 | VGGTAAGHPFYDY | 565 |
| | HFR1 | EVQLLESGGGLVQPGGSLRLSCAAS | 566 |
| | | GFTFS | |
| | HFR2 | WVRQAPGKGLEWVS | 130 |
| | HFR3 | RFTISRDNSKNTLYLQMNSLRAEDS | 567 |
| | | AVYYCAK | |
| | HFR4 | WGQGTLVTVSS | 60 |
| | LC | DIQMTQSPSSLSASVGDRVTITCRA | 568 |
| | | SQSISNYLNWYQQKPGKAPDLLIYA | |
| | | ASSLQSGVPLRFSGSGSGTDFTLTI | |
| | | SSLQPEDFATYYCQQTYSAPTFGGG | |
| | | TKVEIKRTVAAPSVFIFPPSDEQLK | |
| | | SGTASVVCLLNNFYPREAKVQWKVD | |
| | | NALQSGNSQESVTEQDSKDSTYSLS | |
| | | STLTLSKADYE | |
| | LC | DIQMTQSPSSLSASVGDRVTITCRA | 569 |
| | variable | SQSISNYLNWYQQKPGKAPDLLIYA | |
| | | ASSLQSGVPLRFSGSGSGTDFTLTI | |
| | | SSLQPEDFATYYCQQTYSAPTFGGG | |
| | | TKVEIK | |
| | LCDR1 | RASQSISNYLN | 570 |
| | LCDR2 | AASSLQS | 249 |
| | LCDR3 | QQTYSAPT | 571 |
| | LFR1 | DIQMTQSPSSLSASVGDRVTITC | 251 |
| | LFR2 | WYQQKPGKAPDLLIY | 572 |
| | LFR3 | GVPLRFSGSGSGTDFTLTISSLQPE | 573 |
| | | DFATYYC | |
| | LFR4 | FGGGTKVEIK | 85 |
| S144-175 | HC | QVQLVQSGAEVKKPGASVKVSCKAS | 574 |
| | | GYTFTGYYMHWVRQAPGQGLEWMGR | |
| | | INPNSGGTNFAQRFQGRVSMTRDTS | |
| | | ISTAYMELSSLRSDDTAVYYCARGA | |
| | | KFEHLPFDIWGQGTMVTVSSASTKG | |
| | | PSVFPLAPSSKSTSGGTAALGCLVK | |
| | | DYFPEPVTVSWNSGALTSGVHTFPA | |
| | | VLQSSG | |
| | HC | QVQLVQSGAEVKKPGASVKVSCKAS | 575 |
| | variable | GYTFTGYYMHWVRQAPGQGLEWMGR | |
| | | INPNSGGTNFAQRFQGRVSMTRDTS | |
| | | ISTAYMELSSLRSDDTAVYYCARGA | |
| | | KFEHLPFDIWGQGTMVTVSS | |
| | HCDR1 | GYYMH | 187 |
| | HCDR2 | RINPNSGGTNFAQRFQG | 576 |
| | HCDR3 | GAKFEHLPFDI | 577 |
| | HFR1 | QVQLVQSGAEVKKPGASVKVSCKAS | 190 |
| | | GYTFT | |
| | HFR2 | WVRQAPGQGLEWMG | 160 |
| | HFR3 | RVSMTRDTSISTAYMELSSLRSDDT | 578 |
| | | AVYYCAR | |
| | HFR4 | WGQGTMVTVSS | 44 |
| | LC | QSMLTQPPSASGTPGQRVTISCSGS | 579 |
| | | SSNIGSNYVYWYQQLPGTAPKLLIY | |
| | | RNNQRPSGVPDRFSGSKSGTSASLA | |
| | | ISGLRSEDEADYYCAAWDDRRWVFG | |
| | | GGTKLTVLGQPKAAPSVTLFPPSSE | |
| | | ELQANKATLVCLISDFYPGAVTVAW | |
| | | KADSSPVKAGVETTTPSKQSNNKYA | |
| | | ASS | |
| | LC | QSMLTQPPSASGTPGQRVTISCSGS | 580 |
| | variable | SSNIGSNYVYWYQQLPGTAPKLLIY | |
| | | RNNQRPSGVPDRFSGSKSGTSASLA | |
| | | ISGLRSEDEADYYCAAWDDRRWVFG | |
| | | GGTKLTVL | |
| | LCDR1 | SGSSSNIGSNYVY | 403 |
| | LCDR2 | RNNQRPS | 404 |
| | LCDR3 | AAWDDRRWV | 581 |
| | LFR1 | QSMLTQPPSASGTPGQRVTISC | 582 |
| | LFR2 | WYQQLPGTAPKLLIY | 122 |
| | LFR3 | GVPDRFSGSKSGTSASLAISGLRSE | 406 |
| | | DEADYYC | |
| | LFR4 | FGGGTKLTVL | 69 |
| S144-208 | HC | QVQLVQSGAEVKKPGASVKVSCKSS | 583 |
| | | GYTFTGYYMHWVRQAPGQGLEWMGR | |
| | | INPNSGGTNYAQKFQGRVTMTRDTS | |
| | | ISTAYMELSRLRSDDTAVYYCARGA | |
| | | RGGAGCSGWSCFDFWGQGTLVTVSS | |
| | | ASTKGPSVFPLAPSSKSTSGGTAAL | |
| | | GCLVKDYFPEPVTVSWNSGALTSGV | |
| | | HTFPAVLQSSG | |
| | HC | QVQLVQSGAEVKKPGASVKVSCKSS | 584 |
| | variable | GYTFTGYYMHWVRQAPGQGLEWMGR | |
| | | INPNSGGTNYAQKFQGRVTMTRDTS | |
| | | ISTAYMELSRLRSDDTAVYYCARGA | |
| | | RGGAGCSGWSCFDFWGQGTLVTVSS | |
| | HCDR1 | GYYMH | 187 |
| | HCDR2 | RINPNSGGTNYAQKFQG | 585 |
| | HCDR3 | GARGGAGCSGWSCFDF | 586 |
| | HFR1 | QVQLVQSGAEVKKPGASVKVSCKSS | 587 |
| | | GYTFT | |
| | HFR2 | WVRQAPGQGLEWMG | 160 |
| | HFR3 | RVTMTRDTSISTAYMELSRLRSDDT | 588 |
| | | AVYYCAR | |
| | HFR4 | WGQGTLVTVSS | 60 |
| | LC | QSALTQPRSVSGSPGQSVTISCTGT | 589 |
| | | SSDVGGYKYVSWYQQHPGKAPKLMI | |
| | | YDVSKRPSGVPDRFSGSKSGNTASL | |
| | | TISGLQAEDEGDYYCCSYAGTYSLV | |
| | | FGGGTKVTVTVLGQPKAAPSVTLFP | |
| | | PSSEELQANKATLVCLISDFYPGAV | |
| | | TVAWKADSSPVKAGVETTTPSKQSN | |
| | | NKYAASSYLSLTPEQWKSH | |
| | LC | QSALTQPRSVSGSPGQSVTISCTGT | 590 |
| | variable | SSDVGGYKYVSWYQQHPGKAPKLMI | |
| | | YDVSKRPSGVPDRFSGSKSGNTASL | |
| | | TISGLQAEDEGDYYCCSYAGTYSLV | |
| | | FGGGTKVTV | |
| | LCDR1 | TGTSSDVGGYKYVS | 591 |
| | LCDR2 | DVSKRPS | 592 |
| | LCDR3 | CSYAGTYSLV | 593 |
| | LFR1 | QSALTQPRSVSGSPGQSVTISC | 594 |
| | LFR2 | WYQQHPGKAPKLMIY | 67 |
| | LFR3 | GVPDRFSGSKSGNTASLTISGLQAE | 595 |
| | | DEGDYYC | |
| | LFR4 | FGGGTKVTV | 596 |
| S144-339 | HC | EVQLVESGGGLVKPGGSLRLSCAAS | 597 |
| | | GFTFSDYTMNWVRQAPGKGLEWVSS | |
| | | ITRSSTYIYYADSVKGRFTISRDNA | |
| | | KNSLYLQMNSLRAEDTAVYYCARDP | |
| | | YYDILTGYWNYWGQGTLVTVSSAST | |
| | | KGPSVFPLAPSSKSTSGGTAALGCL | |
| | | VKDYFPEPVTVSWNSGALTSGVHTF | |
| | | PAVLQSSG | |
| | HC | EVQLVESGGGLVKPGGSLRLSCAAS | 598 |
| | variable | GFTFSDYTMNWVRQAPGKGLEWVSS | |
| | | ITRSSTYIYYADSVKGRFTISRDNA | |
| | | KNSLYLQMNSLRAEDTAVYYCARDP | |
| | | YYDILTGYWNYWGQGTLVTVSS | |
| | HCDR1 | DYTMN | 599 |
| | HCDR2 | SITRSSTYIYYADSVKG | 600 |
| | HCDR3 | DPYYDILTGYWNY | 601 |
| | HFR1 | EVQLVESGGGLVKPGGSLRLSCAAS | 602 |
| | | GFTFS | |
| | HFR2 | WVRQAPGKGLEWVS | 130 |
| | HFR3 | RFTISRDNAKNSLYLQMNSLRAEDT | 273 |
| | | AVYYCAR | |
| | HFR4 | WGQGTLVTVSS | 60 |
| | LC | EIVLTQSPGTLSLSPGERATLSCRA | 603 |
| | | SQSLSSSYLAWYQQKPGQSPRLLIY | |
| | | GASSRATGIPDRFSGSGSGTDFTLT | |
| | | INRLEPEDFAVYYCQQYRTSPRGTF | |
| | | GGGTKVEIKRTVAAPSVFIFPPSDE | |
| | | QLKSGTASVVCLLNNFYPREAKVQW | |
| | | KVDNALQSGNSQESVTEQDSKDSTY | |
| | | SLSSTLTLSKADYE | |
| | LC | EIVLTQSPGTLSLSPGERATLSCRA | 604 |
| | variable | SQSLSSSYLAWYQQKPGQSPRLLIY | |
| | | GASSRATGIPDRFSGSGSGTDFTLT | |
| | | INRLEPEDFAVYYCQQYRTSPRGTF | |
| | | GGGTKVEIK | |
| | LCDR1 | RASQSLSSSYLA | 605 |
| | LCDR2 | GASSRAT | 135 |
| | LCDR3 | QQYRTSPRGT | 606 |
| | LFR1 | EIVLTQSPGTLSLSPGERATLSC | 50 |
| | LFR2 | WYQQKPGQSPRLLIY | 607 |
| | LFR3 | GIPDRFSGSGSGTDFTLTINRLEPE | 138 |
| | | DFAVYYC | |
| | LFR4 | FGGGTKVEIK | 85 |
| S144-359 | HC | EVQLVESGGGLVQPGGSLRLSCAAS | 608 |
| | | GFTFSSYAMSWVRQAPGKGLEWVSS | |
| | | IRGSGGSTYYADSVKGRFTISRDNS | |
| | | KYTLYLQMNSLRAEDTAVYYCAKIT | |
| | | GAVGGENWFDPWGQGTLVTVSSAST | |
| | | KGPSVFPLAPCSRSTSGGTAALGCL | |
| | | VKDYFPEPVTVSWNSGALTSGVHTF | |
| | | PAVLQSSG | |
| | HC | EVQLVESGGGLVQPGGSLRLSCAAS | 609 |
| | variable | GFTFSSYAMSWVRQAPGKGLEWVSS | |
| | | IRGSGGSTYYADSVKGRFTISRDNS | |
| | | KYTLYLQMNSLRAEDTAVYYCAKIT | |
| | | GAVGGENWFDPWGQGTLVTVSS | |
| | HCDR1 | SYAMS | 610 |
| | HCDR2 | SIRGSGGSTYYADSVKG | 611 |
| | HCDR3 | ITGAVGGENWFDP | 612 |
| | HFR1 | EVQLVESGGGLVQPGGSLRLSCAAS | 613 |
| | | GFTFS | |
| | HFR2 | WVRQAPGKGLEWVS | 130 |
| | HFR3 | RFTISRDNSKYTLYLQMNSLRAEDT | 614 |
| | | AVYYCAK | |
| | HFR4 | WGQGTLVTVSS | 60 |
| | LC | DIQMTQSPSSLSASVGDRVTITCRA | 615 |
| | | SQSISSYLNWYQQKPGKAPKLLIYA | |
| | | ASSLQSGVPSRFSGSGSGTDFTLTI | |
| | | SSLQPEDFAIYYCQQTSRTPLTFGG | |
| | | GTKVEVKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | DIQMTQSPSSLSASVGDRVTITCRA | 616 |
| | variable | SQSISSYLNWYQQKPGKAPKLLIYA | |
| | | ASSLQSGVPSRFSGSGSGTDFTLTI | |
| | | SSLQPEDFAIYYCQQTSRTPLTFGG | |
| | | GTKVEVK | |
| | LCDR1 | RASQSISSYLN | 248 |
| | LCDR2 | AASSLQS | 249 |
| | LCDR3 | QQTSRTPLT | 617 |
| | LFR1 | DIQMTQSPSSLSASVGDRVTITC | 251 |
| | LFR2 | WYQQKPGKAPKLLIY | 252 |
| | LFR3 | GVPSRFSGSGSGTDFTLTISSLQPE | 618 |
| | | DFAIYYC | |
| | LFR4 | FGGGTKVEVK | 619 |
| S144-460 | HC | EVRLVQSGGGLVKPGGSLRLSCAAS | 620 |
| | | GFTFSTAWVRWVRQAPGKGLECVGR | |
| | | IKSKNDGDRAEYAAPARGRFIISRD | |
| | | DAENILYLQMNNLKTEDTAFYYCTT | |
| | | DQGNSSAFYSADYWGQGTLVTVSSA | |
| | | SPTSPKVFPLSLDSTPQDGNVVVAC | |
| | | LVQGFFPQEPLSVTWSESGQNVTAR | |
| | | NF | |
| | HC | EVRLVQSGGGLVKPGGSLRLSCAAS | 621 |
| | variable | GFTFSTAWVRWVRQAPGKGLECVGR | |
| | | IKSKNDGDRAEYAAPARGRFIISRD | |
| | | DAENILYLQMNNLKTEDTAFYYCTT | |
| | | DQGNSSAFYSADYWGQGTLVTVSS | |
| | HCDR1 | TAWVR | 622 |
| | HCDR2 | RIKSKNDGDRAEYAAPARG | 623 |
| | HCDR3 | DQGNSSAFYSADY | 624 |
| | HFR1 | EVRLVQSGGGLVKPGGSLRLSCAAS | 625 |
| | | GFTFS | |
| | HFR2 | WVRQAPGKGLECVG | 626 |
| | HFR3 | RFIISRDDAENILYLQMNNLKTEDT | 627 |
| | | AFYYCTT | |
| | HFR4 | WGQGTLVTVSS | 60 |
| | LC | DIQMTQSPSAMSASVGDRVTITCRA | 628 |
| | | SQDINTFLTWFQQKPGKVPQRLIFA | |
| | | AYRLQSGVPSRFSGSGSGTEFTLTI | |
| | | NSLQPEDVATYYCLHHKTYPYTFGQ | |
| | | GTKLEIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | DIQMTQSPSAMSASVGDRVTITCRA | 629 |
| | variable | SQDINTFLTWFQQKPGKVPQRLIFA | |
| | | AYRLQSGVPSRFSGSGSGTEFTLTI | |
| | | NSLQPEDVATYYCLHHKTYPYTFGQ | |
| | | GTKLEIK | |
| | LCDR1 | RASQDINTFLT | 630 |
| | LCDR2 | AAYRLQS | 631 |
| | LCDR3 | LHHKTYPYT | 632 |
| | LFR1 | DIQMTQSPSAMSASVGDRVTITC | 633 |
| | LFR2 | WFQQKPGKVPQRLIF | 634 |
| | LFR3 | GVPSRFSGSGSGTEFTLTINSLQPE | 635 |
| | | DVATYYC | |
| | LFR4 | FGQGTKLEIK | 380 |
| S144-466 | HC | EVQLVQSGAEVKKPGESLKISCKGS | 636 |
| | | GYRFTRYWIGWVRQMPGKGLEWMGI | |
| | | IYLGDSETRYSPSFQGQVTISADNS | |
| | | ISTAYLQWSSLKASDTAMYYCARSS | |
| | | NWNYGDYWGQGTLVTVSSASTKGPS | |
| | | VFPLAPCSRSTSGGTAALGCLVKDY | |
| | | FPEPVTVSWNSGALTSGVHTFPAVL | |
| | | QSSG | |
| | HC | EVQLVQSGAEVKKPGESLKISCKGS | 637 |
| | variable | GYRFTRYWIGWVRQMPGKGLEWMGI | |
| | | IYLGDSETRYSPSFQGQVTISADNS | |
| | | ISTAYLQWSSLKASDTAMYYCARSS | |
| | | NWNYGDYWGQGTLVTVSS | |
| | HCDR1 | RYWIG | 638 |
| | HCDR2 | IIYLGDSETRYSPSFQG | 639 |
| | HCDR3 | SSNWNYGDY | 640 |
| | HFR1 | EVQLVQSGAEVKKPGESLKISCKGS | 641 |
| | | GYRFT | |
| | HFR2 | WVRQMPGKGLEWMG | 174 |
| | HFR3 | QVTISADNSISTAYLQWSSLKASDT | 642 |
| | | AMYYCAR | |
| | HFR4 | WGQGTLVTVSS | 60 |
| | LC | DIQMTQSPSTLSASVGDRVTITCRA | 643 |
| | | SQSITSWLAWYQQKSGKAPKLLIYD | |
| | | ASSLESGVPSRFSGSGSGTEFTLTI | |
| | | SSLQPDDFATYYCQQYNSYPWTFGQ | |
| | | GTKVEIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | DIQMTQSPSTLSASVGDRVTITCRA | 644 |
| | variable | SQSITSWLAWYQQKSGKAPKLLIYD | |
| | | ASSLESGVPSRFSGSGSGTEFTLTI | |
| | | SSLQPDDFATYYCQQYNSYPWTFGQ | |
| | | GTKVEIK | |
| | LCDR1 | RASQSITSWLA | 645 |
| | LCDR2 | DASSLES | 387 |
| | LCDR3 | QQYNSYPWT | 646 |
| | LFR1 | DIQMTQSPSTLSASVGDRVTITC | 523 |
| | LFR2 | WYQQKSGKAPKLLIY | 647 |
| | LFR3 | GVPSRFSGSGSGTEFTLTISSLQPD | 524 |
| | | DFATYYC | |
| | LFR4 | FGQGTKVEIK | 53 |
| S144-469 | HC | QVQLQESGPGLVKPSETLSLTCTVS | 648 |
| | | GGSISSDYWSWIRQPPGKGLEWIGY | |
| | | MYYSGSTNYNPSLKSRVTISVDTSK | |
| | | NQFSLKLSSVTAADTAVYYCARWDR | |
| | | GSRPHYYYYGMDVWGQGTTVTVSSA | |
| | | STKGPSVFPLAPSSKSTSGGTAALG | |
| | | CLVKDYFPEPVTVSWNSGALTSGVH | |
| | | TFPAVLQSSG | |
| | HC | QVQLQESGPGLVKPSETLSLTCTVS | 649 |
| | variable | GGSISSDYWSWIRQPPGKGLEWIGY | |
| | | MYYSGSTNYNPSLKSRVTISVDTSK | |
| | | NQFSLKLSSVTAADTAVYYCARWDR | |
| | | GSRPHYYYYGMDVWGQGTTVTVSS | |
| | HCDR1 | SDYWS | 650 |
| | HCDR2 | YMYYSGSTNYNPSLKS | 88 |
| | HCDR3 | WDRGSRPHYYYYGMDV | 651 |
| | HFR1 | QVQLQESGPGLVKPSETLSLTCTVS | 24 |
| | | GGSIS | |
| | HFR2 | WIRQPPGKGLEWIG | 7 |
| | HFR3 | RVTISVDTSKNQFSLKLSSVTAADT | 115 |
| | | AVYYCAR | |
| | HFR4 | WGQGTTVTVSS | 147 |
| | LC | DIVMTQSPLSLPVTPGEPASISCRS | 652 |
| | | SQSLLHSNGYNYLDWYLQKPGQSPQ | |
| | | LLIYLGSNRASGVPDRFSGSASGTD | |
| | | FTLKISRVEAEDVGVYYCMQALQAF | |
| | | TFGPGTKVDIKRTVAAPSVFIFPPS | |
| | | DEQLKSGTASVVCLLNNFYPREAKV | |
| | | QWKVDNALQSGNSQESVTEQDSKDS | |
| | | TYSLSSTLTLSKADYE | |
| | LC | DIVMTQSPLSLPVTPGEPASISCRS | 653 |
| | variable | SQSLLHSNGYNYLDWYLQKPGQSPQ | |
| | | LLIYLGSNRASGVPDRFSGSASGTD | |
| | | FTLKISRVEAEDVGVYYCMQALQAF | |
| | | TFGPGTKVDIK | |
| | LCDR1 | RSSQSLLHSNGYNYLD | 262 |
| | LCDR2 | LGSNRAS | 263 |
| | LCDR3 | MQALQAFT | 654 |
| | LFR1 | DIVMTQSPLSLPVTPGEPASISC | 265 |
| | LFR2 | WYLQKPGQSPQLLIY | 266 |
| | LFR3 | GVPDRFSGSASGTDFTLKISRVEAE | 655 |
| | | DVGVYYC | |
| | LFR4 | FGPGTKVDIK | 443 |
| S144-509 | HC | EVQLVQSGAEVKKPGESLKISCKGS | 656 |
| | | AYTFTTYWIGWVRQMPGKGLEWMGI | |
| | | IYPGDSDTRYSPSFQGQVTISADKS | |
| | | ISTAYLQWSSLKASDTAMYYCARLL | |
| | | LVAGPFDYWGQGTLVTVSSASTKGP | |
| | | SVFPLAPSSKSTSGGTAALGCLVKD | |
| | | YFPEPVTVSWNSGALTSGVHTFPAV | |
| | | LQSSGLYSLSSVVTVPSSSLGTQTY | |
| | | ICNVNHKPSNTKVD | |
| | HC | EVQLVQSGAEVKKPGESLKISCKGS | 657 |
| | variable | AYTFTTYWIGWVRQMPGKGLEWMGI | |
| | | IYPGDSDTRYSPSFQGQVTISADKS | |
| | | ISTAYLQWSSLKASDTAMYYCARLL | |
| | | LVAGPFDYWGQGTLVTVSS | |
| | HCDR1 | TYWIG | 658 |
| | HCDR2 | IIYPGDSDTRYSPSFQG | 543 |
| | HCDR3 | LLLVAGPFDY | 659 |
| | HFR1 | EVQLVQSGAEVKKPGESLKISCKGS | 660 |
| | | AYTFT | |
| | HFR2 | WVRQMPGKGLEWMG | 174 |
| | HFR3 | QVTISADKSISTAYLQWSSLKASDT | 661 |
| | | AMYYCAR | |
| | HFR4 | WGQGTLVTVSS | 60 |
| | LC | DIQMTQSPSTLSASVGDRVTITCRA | 662 |
| | | SQSISSWLAWYQQKPGKAPNLLIYD | |
| | | ASSLESGVPSRFSGSGSGTEFTLTI | |
| | | SSLQPDDFATYYCQQYNSYPWTFGQ | |
| | | GTKVEIKRTVAAPSVFIFPPSDEQL | |
| | | KSGTASVVCLLNNFYPREAKVQWKV | |
| | | DN | |
| | LC | DIQMTQSPSTLSASVGDRVTITCRA | 663 |
| | variable | SQSISSWLAWYQQKPGKAPNLLIYD | |
| | | ASSLESGVPSRFSGSGSGTEFTLTI | |
| | | SSLQPDDFATYYCQQYNSYPWTFGQ | |
| | | GTKVEIK | |
| | LCDR1 | RASQSISSWLA | 520 |
| | LCDR2 | DASSLES | 387 |
| | LCDR3 | QQYNSYPWT | 646 |
| | LFR1 | DIQMTQSPSTLSASVGDRVTITC | 523 |
| | LFR2 | WYQQKPGKAPNLLIY | 664 |
| | LFR3 | GVPSRFSGSGSGTEFTLTISSLQPD | 524 |
| | | DFATYYC | |
| | LFR4 | FGQGTKVEIK | 53 |
| S144-516 | HC | QVQLLQSGAEVKKPGASVKVSCKAS | 665 |
| | | GYTFTGYYMHWVRQAPGQGLEWMGR | |
| | | INPNSGGTNYAQKFQGRVTMTRDTS | |
| | | ISTAYMELSRLTSDDTAVYYCATKT | |
| | | GIDRYYYYYMDVWGKGTTVTVSSAS | |
| | | TKGPSVFPLAPSSKSTSGGTAALGC | |
| | | LVKDYFPEPVTVSWNSGALTSGVHT | |
| | | FPAVLQSSG | |
| | HC | QVQLLQSGAEVKKPGASVKVSCKAS | 666 |
| | variable | GYTFTGYYMHWVRQAPGQGLEWMGR | |
| | | INPNSGGTNYAQKFQGRVTMTRDTS | |
| | | ISTAYMELSRLTSDDTAVYYCATKT | |
| | | GIDRYYYYYMDVWGKGTTVTVSS | |
| | HCDR1 | GYYMH | 187 |
| | HCDR2 | RINPNSGGTNYAQKFQG | 585 |
| | HCDR3 | KTGIDRYYYYYMDV | 667 |
| | HFR1 | QVQLLQSGAEVKKPGASVKVSCKAS | 668 |
| | | GYTFT | |
| | HFR2 | WVRQAPGQGLEWMG | 160 |
| | HFR3 | RVTMTRDTSISTAYMELSRLTSDDT | 669 |
| | | AVYYCAT | |
| | HFR4 | WGKGTTVTVSS | 670 |
| | LC | QSVLTQPPSVSEAPGQRVTISCTGS | 671 |
| | | SSNIGAGYDVHWYQQLPGTAPKLLI | |
| | | YGNINRPSGVPDRFSGSKSGTSASL | |
| | | AITGLQAEDEADYYCQSYDNSLNGS | |
| | | VFGGGTKLTVLRQPKAAPSVTLFPP | |
| | | SSEELQANKATLVCLISDFYPGAVT | |
| | | VAWKADSSPVKAGVETTTPSKQSNN | |
| | | KYAASS | |
| | LC | QSVLTQPPSVSEAPGQRVTISCTGS | 672 |
| | variable | SSNIGAGYDVHWYQQLPGTAPKLLI | |
| | | YGNINRPSGVPDRFSGSKSGTSASL | |
| | | AITGLQAEDEADYYCQSYDNSLNGS | |
| | | VFGGGTKLTVL | |
| | LCDR1 | TGSSSNIGAGYDVH | 673 |
| | LCDR2 | GNINRPS | 674 |
| | LCDR3 | QSYDNSLNGSV | 675 |
| | LFR1 | QSVLTQPPSVSEAPGQRVTISC | 676 |
| | LFR2 | WYQQLPGTAPKLLIY | 122 |
| | LFR3 | GVPDRFSGSKSGTSASLAITGLQAE | 539 |
| | | DEADYYC | |
| | LFR4 | FGGGTKLTVL | 69 |
| S144-568 | HC | QVQLQESGPGLVKPSETLSLTCSVS | 677 |
| | | GGSISDYYWSWIRQPPGKGLEWIGY | |
| | | IYNSGSTNYNPSLKSRVTISADPSK | |
| | | NQFSLKLSSVTAADTAVYYCARPHG | |
| | | GDYAFDIWGQGTMVTVSSASPTSPK | |
| | | VFPLSLDSTPQDGNVVVACLVQGFF | |
| | | PQEPLSVTWSESGQNVTARNF | |
| | HC | QVQLQESGPGLVKPSETLSLTCSVS | 678 |
| | variable | GGSISDYYWSWIRQPPGKGLEWIGY | |
| | | IYNSGSTNYNPSLKSRVTISADPSK | |
| | | NQFSLKLSSVTAADTAVYYCARPHG | |
| | | GDYAFDIWGQGTMVTVSS | |
| | HCDR1 | DYYWS | 679 |
| | HCDR2 | YIYNSGSTNYNPSLKS | 680 |
| | HCDR3 | PHGGDYAFDI | 681 |
| | HFR1 | QVQLQESGPGLVKPSETLSLTCSVS | 682 |
| | | GGSIS | |
| | HFR2 | WIRQPPGKGLEWIG | 7 |
| | HFR3 | RVTISADPSKNQFSLKLSSVTAADT | 683 |
| | | AVYYCAR | |
| | HFR4 | WGQGTMVTVSS | 44 |
| | LC | EIVLTQSPGTLSLSPGERATLSCRA | 684 |
| | | SQSVSSNFLAWYQQKPGQPPRLLIY | |
| | | GASVRATGIPDRFSGSGSGTDFTLT | |
| | | ITRLEPEDFAVYYCQQYGSLPRTFG | |
| | | QGTKVEIKRTVAAPSVFIFPPSDEQ | |
| | | LKSGTASVVCLLNNFYPREAKVQWK | |
| | | VDNALQSGNSQESVTEQDSKDSTYS | |
| | | LSSTLTLSKADYE | |
| | LC | EIVLTQSPGTLSLSPGERATLSCRA | 685 |
| | variable | SQSVSSNFLAWYQQKPGQPPRLLIY | |
| | | GASVRATGIPDRFSGSGSGTDFTLT | |
| | | ITRLEPEDFAVYYCQQYGSLPRTFG | |
| | | QGTKVEIK | |
| | LCDR1 | RASQSVSSNFLA | 686 |
| | LCDR2 | GASVRAT | 687 |
| | LCDR3 | QQYGSLPRT | 688 |
| | LFR1 | EIVLTQSPGTLSLSPGERATLSC | 50 |
| | LFR2 | WYQQKPGQPPRLLIY | 689 |
| | LFR3 | GIPDRFSGSGSGTDFTLTITRLEPE | 690 |
| | | DFAVYYC | |
| | LFR4 | FGQGTKVEIK | 53 |
| S144-576 | HC | QVQLVQSGAEVMKPGSSVKVSCKAS | 691 |
| | | GGTFSSYSITWVRQAPGQGLEWMGR | |
| | | IIPILGIANYAQKFQGRVTITADKS | |
| | | TSTAYMELSSLRSEDTAVYYCARGY | |
| | | SGSPSNLDGMDVWGQGTTVTVSSAS | |
| | | TKGPSVFPLAPSSKSTSGGTAALGC | |
| | | LVKDYFPEPVTVSWNSGALTSGVHT | |
| | | FPAVLQSSG | |
| | HC | QVQLVQSGAEVMKPGSSVKVSCKAS | 692 |
| | variable | GGTFSSYSITWVRQAPGQGLEWMGR | |
| | | IIPILGIANYAQKFQGRVTITADKS | |
| | | TSTAYMELSSLRSEDTAVYYCARGY | |
| | | SGSPSNLDGMDVWGQGTTVTVSS | |
| | HCDR1 | SYSIT | 693 |
| | HCDR2 | RIIPILGIANYAQKFQG | 157 |
| | HCDR3 | GYSGSPSNLDGMDV | 694 |
| | HFR1 | QVQLVQSGAEVMKPGSSVKVSCKAS | 695 |
| | | GGTFS | |
| | HFR2 | WVRQAPGQGLEWMG | 160 |
| | HFR3 | RVTITADKSTSTAYMELSSLRSEDT | 161 |
| | | AVYYCAR | |
| | HFR4 | WGQGTTVTVSS | 147 |
| | LC | IQMTQSPSTLSASVGDRVTITCRAS | 696 |
| | | QSISSWLAWYQQKPGKAPKLLIYDA | |
| | | SSLQSGVPSRFSGSGSGTEFTLTIS | |
| | | SLQPDDFATYYCQQYNSYSPITFGQ | |
| | | GTRLEIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | IQMTQSPSTLSASVGDRVTITCRAS | 697 |
| | variable | QSISSWLAWYQQKPGKAPKLLIYDA | |
| | | SSLQSGVPSRFSGSGSGTEFTLTIS | |
| | | SLQPDDFATYYCQQYNSYSPITFGQ | |
| | | GTRLEIK | |
| | LCDR1 | RASQSISSWLA | 520 |
| | LCDR2 | DASSLQS | 698 |
| | LCDR3 | QQYNSYSPIT | 699 |
| | LFR1 | IQMTQSPSTLSASVGDRVTITC | 700 |
| | LFR2 | WYQQKPGKAPKLLIY | 252 |
| | LFR3 | GVPSRFSGSGSGTEFTLTISSLQPD | 524 |
| | | DFATYYC | |
| | LFR4 | FGQGTRLEIK | 701 |
| S144-588 | HC | QLQLQESGPGLVKPSETLSLTCTVS | 702 |
| | | GGSISSSSYYWGWIRQPPGKGLEWI | |
| | | GSIYYSGSTYYNPSLKSRFTISVDT | |
| | | SKNQFSLKLSSVTAADTAVYYCAAY | |
| | | QRKLGYCRGNSCFSCFDPWGQGTLV | |
| | | TVSSASTKGPSVFPLAPSSKSTSGG | |
| | | TAALGCLVKDYFPEPVTVSWNSGAL | |
| | | TSGVHTFPAVLQSSG | |
| | HC | QLQLQESGPGLVKPSETLSLTCTVS | 703 |
| | variable | GGSISSSSYYWGWIRQPPGKGLEWI | |
| | | GSIYYSGSTYYNPSLKSRFTISVDT | |
| | | SKNQFSLKLSSVTAADTAVYYCAAY | |
| | | QRKLGYCRGNSCFSCFDPWGQGTLV | |
| | | TVSS | |
| | HCDR1 | SSSYYWG | 242 |
| | HCDR2 | SIYYSGSTYYNPSLKS | 243 |
| | HCDR3 | YQRKLGYCRGNSCFSCFDP | 704 |
| | HFR1 | QLQLQESGPGLVKPSETLSLTCTVS | 245 |
| | | GGSIS | |
| | HFR2 | WIRQPPGKGLEWIG | 7 |
| | HFR3 | RFTISVDTSKNQFSLKLSSVTAADT | 705 |
| | | AVYYCAA | |
| | HFR4 | WGQGTLVTVSS | 60 |
| | LC | SYELTQPPSVSVSPGQTASITCSGD | 706 |
| | | KLGDKYACWYQQKPGQSPVLVIYQD | |
| | | TKRPSGIPERFSGSNSGNTATLTIS | |
| | | GTQAMDEADYYCQAWDSSTVLFGGG | |
| | | TKLTVLGQPKAAPSVTLFPPSSEEL | |
| | | QANKATLVCLISDFYPGAVTVAWKA | |
| | | DSSPVKAGVETTTPSKQSNNKYAAS | |
| | | SYLSLTPEQWKSH | |
| | LC | SYELTQPPSVSVSPGQTASITCSGD | 707 |
| | variable | KLGDKYACWYQQKPGQSPVLVIYQD | |
| | | TKRPSGIPERFSGSNSGNTATLTIS | |
| | | GTQAMDEADYYCQAWDSSTVLFGGG | |
| | | TKLTVL | |
| | LCDR1 | SGDKLGDKYAC | 502 |
| | LCDR2 | QDTKRPS | 366 |
| | LCDR3 | QAWDSSTVL | 708 |
| | LFR1 | SYELTQPPSVSVSPGQTASITC | 368 |
| | LFR2 | WYQQKPGQSPVLVIY | 369 |
| | LFR3 | GIPERFSGSNSGNTATLTISGTQAM | 370 |
| | | DEADYYC | |
| | LFR4 | FGGGTKLTVL | 69 |
| S144-628 | HC | EVHLVQSGAEVKQPGESLKISCKGS | 709 |
| | | GYNFATYWIAWVRQMPGKGLEWMGI | |
| | | IYPGDSDTRYSPSFQGQVIISADKS | |
| | | IGTAFLQWSSLKASDTAMYYCARRG | |
| | | YSSSNYRVDEYYYYGMDVWGQGTTV | |
| | | TVSSASPTSPKVFPLSLCSTQPDGN | |
| | | VVIACLVQGFFPQEPLSVTWSESGQ | |
| | | GVTARNFP | |
| | HC | EVHLVQSGAEVKQPGESLKISCKGS | 710 |
| | variable | GYNFATYWIAWVRQMPGKGLEWMGI | |
| | | IYPGDSDTRYSPSFQGQVIISADKS | |
| | | IGTAFLQWSSLKASDTAMYYCARRG | |
| | | YSSSNYRVDEYYYYGMDVWGQGTTV | |
| | | TVSS | |
| | HCDR1 | TYWIA | 527 |
| | HCDR2 | IIYPGDSDTRYSPSFQG | 543 |
| | HCDR3 | RGYSSSNYRVDEYYYYGMDV | 711 |
| | HFR1 | EVHLVQSGAEVKQPGESLKISCKGS | 712 |
| | | GYNFA | |
| | HFR2 | WVRQMPGKGLEWMG | 174 |
| | HFR3 | QVIISADKSIGTAFLQWSSLKASDT | 713 |
| | | AMYYCAR | |
| | HFR4 | WGQGTTVTVSS | 147 |
| | LC | QSVLTQPPSMSGAPGQRVTISCTGS | 714 |
| | | SSNIGAGYDVHWYQQLPGAAPKLLI | |
| | | YGDTSRPSGVPDRFSGSKSDTSASL | |
| | | AITGLQAEDEADYYCQSFDRSLSGL | |
| | | VIFGGGTRLTVLGQPKAAPSVTLFP | |
| | | PSSEELQANKATLVCLISDFYPGAV | |
| | | TVAWKADSSPVKAGVETTTPSKQSN | |
| | | NKYAASS*DRKS | |
| | LC | QSVLTQPPSMSGAPGQRVTISCTGS | 715 |
| | variable | SSNIGAGYDVHWYQQLPGAAPKLLI | |
| | | YGDTSRPSGVPDRFSGSKSDTSASL | |
| | | AITGLQAEDEADYYCQSFDRSLSGL | |
| | | VIFGGGTRLTVL | |
| | LCDR1 | TGSSSNIGAGYDVH | 673 |
| | LCDR2 | GDTSRPS | 716 |
| | LCDR3 | QSFDRSLSGLVI | 717 |
| | LFR1 | QSVLTQPPSMSGAPGQRVTISC | 718 |
| | LFR2 | WYQQLPGAAPKLLIY | 719 |
| | LFR3 | GVPDRFSGSKSDTSASLAITGLQAE | 720 |
| | | DEADYYC | |
| | LFR4 | FGGGTRLTVL | 721 |
| S144-740 | HC | QVQLVQSGAEVKKPGASVKVSCKAS | 722 |
| | | GYTFTGYYMHWVRQAPGQGLEWMGR | |
| | | INPNSGDTNYAQKFQGRVTMTRDTS | |
| | | ISTAYMELSRLRSDDTAVYYCARLG | |
| | | KGMAAARTVFDSWGQGTLVTVSSAS | |
| | | TKGPSVFPLAPSSKSTSGGTAALGC | |
| | | LVKDYFPEPVTVSWNSGALTSGVHT | |
| | | FPAVLQSSG | |
| | HC | QVQLVQSGAEVKKPGASVKVSCKAS | 723 |
| | variable | GYTFTGYYMHWVRQAPGQGLEWMGR | |
| | | INPNSGDTNYAQKFQGRVTMTRDTS | |
| | | ISTAYMELSRLRSDDTAVYYCARLG | |
| | | KGMAAARTVFDSWGQGTLVTVSS | |
| | HCDR1 | GYYMH | 187 |
| | HCDR2 | RINPNSGDTNYAQKFQG | 724 |
| | HCDR3 | LGKGMAAARTVFDS | 725 |
| | HFR1 | QVQLVQSGAEVKKPGASVKVSCKAS | 190 |
| | | GYTFT | |
| | HFR2 | WVRQAPGQGLEWMG | 160 |
| | HFR3 | RVTMTRDTSISTAYMELSRLRSDDT | 588 |
| | | AVYYCAR | |
| | HFR4 | WGQGTLVTVSS | 60 |
| | LC | EVVLTQSPGTLSLSPGERATLSCRA | 726 |
| | | SQSVSSSYLAWYQQKPGQAPRLVIY | |
| | | GASSRATGIPDRFSGSGSGTDFTLT | |
| | | ISRLEPEDFAVYYCQQFGSSPTFGR | |
| | | GTRLEIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | EVVLTQSPGTLSLSPGERATLSCRA | 727 |
| | variable | SQSVSSSYLAWYQQKPGQAPRLVIY | |
| | | GASSRATGIPDRFSGSGSGTDFTLT | |
| | | ISRLEPEDFAVYYCQQFGSSPTFGR | |
| | | GTRLEIK | |
| | LCDR1 | RASQSVSSSYLA | 338 |
| | LCDR2 | GASSRAT | 135 |
| | LCDR3 | QQFGSSPT | 728 |
| | LFR1 | EVVLTQSPGTLSLSPGERATLSC | 729 |
| | LFR2 | WYQQKPGQAPRLVIY | 730 |
| | LFR3 | GIPDRFSGSGSGTDFTLTISRLEPE | 196 |
| | | DFAVYYC | |
| | LFR4 | FGRGTRLEIK | 731 |
| S144-741 | HC | QVHLVQSGAEVKKPGASVKVSCKAS | 732 |
| | | GYTFTGYYMNWVRQAPGQGLEWMGR | |
| | | INPNSGGTNYAQKFQGRVTMTRDTS | |
| | | ISTAYMELSRLRSDDAAVYYCARAE | |
| | | RYSSSWYNLYYWGQGTLVTVSSAST | |
| | | KGPSVFPLAPSSKSTSGGTAALGCL | |
| | | VKDYFPEPVTVSWNSGALTSGVHTF | |
| | | PAVLQSSG | |
| | HC | QVHLVQSGAEVKKPGASVKVSCKAS | 733 |
| | variable | GYTFTGYYMNWVRQAPGQGLEWMGR | |
| | | INPNSGGTNYAQKFQGRVTMTRDTS | |
| | | ISTAYMELSRLRSDDAAVYYCARAE | |
| | | RYSSSWYNLYYWGQGTLVTVSS | |
| | HCDR1 | GYYMN | 734 |
| | HCDR2 | RINPNSGGTNYAQKFQG | 585 |
| | HCDR3 | AERYSSSWYNLYY | 735 |
| | HFR1 | QVHLVQSGAEVKKPGASVKVSCKAS | 736 |
| | | GYTFT | |
| | HFR2 | WVRQAPGQGLEWMG | 160 |
| | HFR3 | RVTMTRDTSISTAYMELSRLRSDDA | 737 |
| | | AVYYCAR | |
| | HFR4 | WGQGTLVTVSS | 60 |
| | LC | QSVLTQPPSASGTPGQRVTISCSGS | 738 |
| | | SSNIGSNTVNWYQQLPGTAPKLLIY | |
| | | SNNQRPSGVPDRFSGSKSGTSASLA | |
| | | ISGLQSEDEADYYCAAWDDSLNGVV | |
| | | FGGGTKLTVLGQPKAAPSVTLFPPS | |
| | | SEELQANKATLVCLISDFYPGAVTV | |
| | | AWKADSSPVKAGVETTTPSKQSNNK | |
| | | YAASSYLSLTPEQWKSH | |
| | LC | QSVLTQPPSASGTPGQRVTISCSGS | 739 |
| | variable | SSNIGSNTVNWYQQLPGTAPKLLIY | |
| | | SNNQRPSGVPDRFSGSKSGTSASLA | |
| | | ISGLQSEDEADYYCAAWDDSLNGVV | |
| | | FGGGTKLTVL | |
| | LCDR1 | SGSSSNIGSNTVN | 740 |
| | LCDR2 | SNNQRPS | 119 |
| | LCDR3 | AAWDDSLNGVV | 741 |
| | LFR1 | QSVLTQPPSASGTPGQRVTISC | 121 |
| | LFR2 | WYQQLPGTAPKLLIY | 122 |
| | LFR3 | GVPDRFSGSKSGTSASLAISGLQSE | 123 |
| | | DEADYYC | |
| | LFR4 | FGGGTKLTVL | 69 |
| S144-803 | HC | EVQLVQSGAEVKKPGESLKISCKGS | 742 |
| | | RYSFTRYWIAWVRQMPGKGLEWMGI | |
| | | IYPGDSDTRYSPSFQGPVTISADKS | |
| | | ISTAYLQWSSLKASDTAIYYCARLP | |
| | | NSNYVDYWGQGTLVTVSSASTKGPS | |
| | | VFPLAPSSKSTSGGTAALGCLVKDY | |
| | | FPEPVTVSWNSGALTSGVHTFPAVL | |
| | | QSSGLYSLSSVVTVPSSSLGTQTYI | |
| | | CNVNHKPSNTKVD | |
| | HC | EVQLVQSGAEVKKPGESLKISCKGS | 743 |
| | variable | RYSFTRYWIAWVRQMPGKGLEWMGI | |
| | | IYPGDSDTRYSPSFQGPVTISADKS | |
| | | ISTAYLQWSSLKASDTAIYYCARLP | |
| | | NSNYVDYWGQGTLVTVSS | |
| | HCDR1 | RYWIA | 744 |
| | HCDR2 | IIYPGDSDTRYSPSFQG | 543 |
| | HCDR3 | LPNSNYVDY | 745 |
| | HFR1 | EVQLVQSGAEVKKPGESLKISCKGS | 746 |
| | | RYSFT | |
| | HFR2 | WVRQMPGKGLEWMG | 174 |
| | HFR3 | PVTISADKSISTAYLQWSSLKASDT | 747 |
| | | AIYYCAR | |
| | HFR4 | WGQGTLVTVSS | 60 |
| | LC | DIQMTQSPSTLSASVGDRVTITCRA | 748 |
| | | SQSISSWLAWYQQKPGKAPKLLIYD | |
| | | ASSLESGVPSRFSGSGSGTEFTLTI | |
| | | SSLQPDDFATYYCQQYNIYPYTFGQ | |
| | | GTKLDIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | DIQMTQSPSTLSASVGDRVTITCRA | 749 |
| | variable | SQSISSWLAWYQQKPGKAPKLLIYD | |
| | | ASSLESGVPSRFSGSGSGTEFTLTI | |
| | | SSLQPDDFATYYCQQYNIYPYTFGQ | |
| | | GTKLDIK | |
| | LCDR1 | RASQSISSWLA | 520 |
| | LCDR2 | DASSLES | 387 |
| | LCDR3 | QQYNIYPYT | 750 |
| | LFR1 | DIQMTQSPSTLSASVGDRVTITC | 523 |
| | LFR2 | WYQQKPGKAPKLLIY | 252 |
| | LFR3 | GVPSRFSGSGSGTEFTLTISSLQPD | 524 |
| | | DFATYYC | |
| | LFR4 | FGQGTKLDIK | 751 |
| S144-843 | HC | QVQLVESGGGVVQPGGSVRLSCAAS | 752 |
| | | GFDFTNNGMYWVRQAPGKGLEWVAF | |
| | | IRYDGNKQDYADSVKGRFTISRDNS | |
| | | KNTLYLQMSSLRPEDTAVYYCAKGV | |
| | | YTENYGWGQGTLVTVSSGTTVTVSS | |
| | | ASTKGPSVFPLAPCSRSTSESTAAL | |
| | | GCLVKDYFPEPVTVSWNSGALTSGV | |
| | | HTFPAVLQSSGLYSLSSVVTVPSSN | |
| | | FGTQTYTCNVDHKPSNTKVD | |
| | HC | QVQLVESGGGVVQPGGSVRLSCAAS | 753 |
| | variable | GFDFTNNGMYWVRQAPGKGLEWVAF | |
| | | IRYDGNKQDYADSVKGRFTISRDNS | |
| | | KNTLYLQMSSLRPEDTAVYYCAKGV | |
| | | YTENYGWGQGTLVTVSS | |
| | HCDR1 | NNGMY | 754 |
| | HCDR2 | FIRYDGNKQDYADSVKG | 755 |
| | HCDR3 | GVYTENYG | 756 |
| | HFR1 | QVQLVESGGGVVQPGGSVRLSCAAS | 757 |
| | | GFDFT | |
| | HFR2 | WVRQAPGKGLEWVA | 145 |
| | HFR3 | RFTISRDNSKNTLYLQMSSLRPEDT | 758 |
| | | AVYYCAK | |
| | HFR4 | WGQGTLVTVSS | 60 |
| | LC | EIVLTQSPGTLSLSPGERATLSCRA | 759 |
| | | SQTVTSRYLAWYQQKPGQAPRLLIY | |
| | | GASTRATGIPDRFSGSGSGTDFTLT | |
| | | ISRLEPEDFAVYYCQQYGNSPPYTF | |
| | | GQGTKLEIKRTVAAPSVFIFPPSDE | |
| | | QLKSGTASVVCLLNNFYPREAKVQW | |
| | | KVDNALQSGNSQESVTEQDSKDSTY | |
| | | SLSSTLTLSKADYE | |
| | LC | EIVLTQSPGTLSLSPGERATLSCRA | 760 |
| | variable | SQTVTSRYLAWYQQKPGQAPRLLIY | |
| | | GASTRATGIPDRFSGSGSGTDFTLT | |
| | | ISRLEPEDFAVYYCQQYGNSPPYTF | |
| | | GQGTKLEIK | |
| | LCDR1 | RASQTVTSRYLA | 761 |
| | LCDR2 | GASTRAT | 208 |
| | LCDR3 | QQYGNSPPYT | 762 |
| | LFR1 | EIVLTQSPGTLSLSPGERATLSC | 50 |
| | LFR2 | WYQQKPGQAPRLLIY | 182 |
| | LFR3 | GIPDRFSGSGSGTDFTLTISRLEPE | 196 |
| | | DFAVYYC | |
| | LFR4 | FGQGTKLEIK | 380 |
| S144-877 | HC | QVQLVESGGGVVQPGRSLRLSCAAS | 763 |
| | | GFTFSTYGMHWVRQAPGKGLEWVAV | |
| | | ISYDGSNKYYADSVKGRFTISRDNS | |
| | | KNTLYLQMNSLRAEDTAVYYCAKQQ | |
| | | GTYCSGGNCYSGYFDYWGQGTLVTV | |
| | | SSASTKGPSVFPLAPSSKSTSGGTA | |
| | | ALGCLVKDYFPEPVTVSWNSGALTS | |
| | | GVHTFPAVLQSSGLYSLSSVVTVPS | |
| | | SSLGTQTYIC | |
| | HC | QVQLVESGGGVVQPGRSLRLSCAAS | 764 |
| | variable | GFTFSTYGMHWVRQAPGKGLEWVAV | |
| | | ISYDGSNKYYADSVKGRFTISRDNS | |
| | | KNTLYLQMNSLRAEDTAVYYCAKQQ | |
| | | GTYCSGGNCYSGYFDYWGQGTLVTV | |
| | | SS | |
| | HCDR1 | TYGMH | 765 |
| | HCDR2 | VISYDGSNKYYADSVKG | 497 |
| | HCDR3 | QQGTYCSGGNCYSGYFDY | 766 |
| | HFR1 | QVQLVESGGGVVQPGRSLRLSCAAS | 144 |
| | | GFTFS | |
| | HFR2 | WVRQAPGKGLEWVA | 145 |
| | HFR3 | RFTISRDNSKNTLYLQMNSLRAEDT | 499 |
| | | AVYYCAK | |
| | HFR4 | WGQGTLVTVSS | 60 |
| | LC | DIQMTQSPSSLSASVGDRVTITCQA | 767 |
| | | SQDISNYLNWYQQKPGKAPKLLIYD | |
| | | ASNLETGVPSRFSGSGSGTDFSFSI | |
| | | SSLQPEDIATYYCQQYDNVPLTFGG | |
| | | GTKVEIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | DIQMTQSPSSLSASVGDRVTITCQA | 768 |
| | variable | SQDISNYLNWYQQKPGKAPKLLIYD | |
| | | ASNLETGVPSRFSGSGSGTDFSFSI | |
| | | SSLQPEDIATYYCQQYDNVPLTFGG | |
| | | GTKVEIK | |
| | LCDR1 | QASQDISNYLN | 769 |
| | LCDR2 | DASNLET | 770 |
| | LCDR3 | QQYDNVPLT | 771 |
| | LFR1 | DIQMTQSPSSLSASVGDRVTITC | 251 |
| | LFR2 | WYQQKPGKAPKLLIY | 252 |
| | LFR3 | GVPSRFSGSGSGTDFSFSISSLQPE | 772 |
| | | DIATYYC | |
| | LFR4 | FGGGTKVEIK | 85 |
| S144-952 | HC | QVQLVQSGAEVKKPGASVKVSCTAS | 773 |
| | | GYTVTSYGISWVRQAPGQGLEWMGW | |
| | | ISTYNGNTNYAQKLQGRVTMTTDTS | |
| | | TSTAYMELRSLRSDDTAVYYCARE | |
| | | YSYGYRLAYFDYWGQGTLVTVSSGS | |
| | | ASAPTLFPLVSCENSPSDTSSVAVG | |
| | | CLAQDFLPDSITFSWKYKNNSDISS | |
| | | TRGFPSVLRGGKYAATSQVLLPSKD | |
| | | VM | |
| | HC | QVQLVQSGAEVKKPGASVKVSCTAS | 774 |
| | variable | GYTVTSYGISWVRQAPGQGLEWMGW | |
| | | ISTYNGNTNYAQKLQGRVTMTTDTS | |
| | | TSTAYMELRSLRSDDTAVYYCAREY | |
| | | SYGYRLAYFDYWGQGTLVTVSS | |
| | HCDR1 | SYGIS | 775 |
| | HCDR2 | WISTYNGNTNYAQKLQG | 776 |
| | HCDR3 | EYSYGYRLAYFDY | 777 |
| | HFR1 | QVQLVQSGAEVKKPGASVKVSCTAS | 778 |
| | | GYTVT | |
| | HFR2 | WVRQAPGQGLEWMG | 160 |
| | HFR3 | RVTMTTDTSTSTAYMELRSLRSDDT | 779 |
| | | AVYYCAR | |
| | HFR4 | WGQGTLVTVSS | 60 |
| | LC | DIVMTQSPDSLAVSLGERATINCKS | 780 |
| | | SQSVLNSSNNKNYLAWYQQKPGQPP | |
| | | KLLIYWASTRESGVPDRFSGSGSGT | |
| | | DFTLTISSLQAEDVAVYYCQQYYST | |
| | | PQTFGQGTKVEIKRTVAAPSVFIFP | |
| | | PSDEQLKSGTASVVCLLNNFYPREA | |
| | | KVQWKVDNALQSGNSQESVTEQDSK | |
| | | DSTYSLSSTLTLSKADYE | |
| | LC | DIVMTQSPDSLAVSLGERATINCKS | 781 |
| | variable | SQSVLNSSNNKNYLAWYQQKPGQPP | |
| | | KLLIYWASTRESGVPDRFSGSGSGT | |
| | | DFTLTISSLQAEDVAVYYCQQYYST | |
| | | PQTFGQGTKVEIK | |
| | LCDR1 | KSSQSVLNSSNNKNYLA | 782 |
| | LCDR2 | WASTRES | 30 |
| | LCDR3 | QQYYSTPQT | 783 |
| | LFR1 | DIVMTQSPDSLAVSLGERATINC | 32 |
| | LFR2 | WYQQKPGQPPKLLIY | 33 |
| | LFR3 | GVPDRFSGSGSGTDFTLTISSLQAE | 293 |
| | | DVAVYYC | |
| | LFR4 | FGQGTKVEIK | 53 |
| S144-971 | HC | EVQLVESGGGLVQPGGSLRISCSAS | 784 |
| | | GFTFSRYAMHWVRQAPGKGLEYVSA | |
| | | IRSNGGSTYYADSVRGRFTISRDNS | |
| | | RNTLYLQMSSLRAEDTAVYYCVIIN | |
| | | NLAAAGTRFDYWGQGTLVTVSSAST | |
| | | KGPSVFPLAPSSKSTSGGTAALGCL | |
| | | VKDYFPEPVTVSWNSGALTSGVHTF | |
| | | PAVLQSSG | |
| | HC | EVQLVESGGGLVQPGGSLRISCSAS | 785 |
| | variable | GFTFSRYAMHWVRQAPGKGLEYVSA | |
| | | IRSNGGSTYYADSVRGRFTISRDNS | |
| | | RNTLYLQMSSLRAEDTAVYYCVIIN | |
| | | NLAAAGTRFDYWGQGTLVTVSS | |
| | HCDR1 | RYAMH | 786 |
| | HCDR2 | AIRSNGGSTYYADSVRG | 787 |
| | HCDR3 | INNLAAAGTRFDY | 788 |
| | HFR1 | EVQLVESGGGLVQPGGSLRISCSAS | 789 |
| | | GFTFS | |
| | HFR2 | WVRQAPGKGLEYVS | 790 |
| | HFR3 | RFTISRDNSRNTLYLQMSSLRAEDT | 791 |
| | | AVYYCVI | |
| | HFR4 | WGQGTLVTVSS | 60 |
| | LC | DIVMTQSPDSLAVSLGERATINCKS | 792 |
| | | SQSVLYSSNNKNFLTWYQQKPGQPP | |
| | | KLLIYWASTRESGVPDRFSGSGSGT | |
| | | DFTLTISSLQAEDVAVYYCQQYYTT | |
| | | PWTFGQGTKVEIKRTVAAPSVFIFP | |
| | | PSDEQLKSGTASVVCLLNNFYPREA | |
| | | KVQWKVDNALQSGNSQESVTEQDSK | |
| | | DSTYSLSSTLTLSKADYE | |
| | LC | DIVMTQSPDSLAVSLGERATINCKS | 793 |
| | variable | SQSVLYSSNNKNFLTWYQQKPGQPP | |
| | | KLLIYWASTRESGVPDRFSGSGSGT | |
| | | DFTLTISSLQAEDVAVYYCQQYYTT | |
| | | PWTFGQGTKVEIK | |
| | LCDR1 | KSSQSVLYSSNNKNFLT | 794 |
| | LCDR2 | WASTRES | 30 |
| | LCDR3 | QQYYTTPWT | 795 |
| | LFR1 | DIVMTQSPDSLAVSLGERATINC | 32 |
| | LFR2 | WYQQKPGQPPKLLIY | 33 |
| | LFR3 | GVPDRFSGSGSGTDFTLTISSLQAE | 293 |
| | | DVAVYYC | |
| | LFR4 | FGQGTKVEIK | 53 |
| S144- | HC | QVQLQQWGAGLLKPSETLSLTCAVY | 796 |
| 1036 | | GGSFSGYFWSWIRQPPGKGLEWIGE | |
| | | INHSGSTNYNPSLKSRVTISVDTSK | |
| | | NQFSLKLSSVTAADTAVYYCARAPY | |
| | | YDFLREGNWFDPWGQGTLVTVSSAS | |
| | | TKGPSVFPLAPSSKSTSGGTAALGC | |
| | | LVKDYFPEPVTVSWNSGALTSGVHT | |
| | | FPAVLQSSGLYSLSSVVTVPSSSLG | |
| | | TQTYICNVNHKPS | |
| | HC | QVQLQQWGAGLLKPSETLSLTCAVY | 797 |
| | variable | GGSFSGYFWSWIRQPPGKGLEWIGE | |
| | | INHSGSTNYNPSLKSRVTISVDTSK | |
| | | NQFSLKLSSVTAADTAVYYCARAPY | |
| | | YDFLREGNWFDPWGQGTLVTVSS | |
| | HCDR1 | GYFWS | 798 |
| | HCDR2 | EINHSGSTNYNPSLKS | 799 |
| | HCDR3 | APYYDFLREGNWFDP | 800 |
| | HFR1 | QVQLQQWGAGLLKPSETLSLTCAVY | 801 |
| | | GGSFS | |
| | HFR2 | WIRQPPGKGLEWIG | 7 |
| | HFR3 | RVTISVDTSKNQFSLKLSSVTAADT | 115 |
| | | AVYYCAR | |
| | HFR4 | WGQGTLVTVSS | 60 |
| | LC | DIVMTQSPDSLAVSLGERATINCNS | 802 |
| | | SQSVLYSSINKNYLAWYQQKPAQPP | |
| | | KVLIYWASTRESGVPDRFSGSGSGT | |
| | | DFTLTISSLQAEDVAVYYCQQYYRT | |
| | | PWTFGQGTKVEIKRTVAAPSVFIFP | |
| | | PSDEQLKSGTASVVCLLNNFYPREA | |
| | | KVQWKVDNALQSGNSQESVTEQDSK | |
| | | DSTYSLSSTLTLSKADYE | |
| | LC | DIVMTQSPDSLAVSLGERATINCNS | 803 |
| | variable | SQSVLYSSINKNYLAWYQQKPAQPP | |
| | | KVLIYWASTRESGVPDRFSGSGSGT | |
| | | DFTLTISSLQAEDVAVYYCQQYYRT | |
| | | PWTFGQGTKVEIK | |
| | LCDR1 | NSSQSVLYSSINKNYLA | 804 |
| | LCDR2 | WASTRES | 30 |
| | LCDR3 | QQYYRTPWT | 805 |
| | LFR1 | DIVMTQSPDSLAVSLGERATINC | 32 |
| | LFR2 | WYQQKPAQPPKVLIY | 806 |
| | LFR3 | GVPDRFSGSGSGTDFTLTISSLQAE | 293 |
| | | DVAVYYC | |
| | LFR4 | FGQGTKVEIK | 53 |
| S144- | HC | QVQLVQSGAEVKKPGSSVKVSCKAS | 807 |
| 1079 | | GDTFGSYSITWVRQAPGQGLEWMGR | |
| | | IIPVLGIANYAQKFQGRVTITADKS | |
| | | TSTAYMELSSLRSEDTAVYYCAGGG | |
| | | CSGGNCYSWYNWFDPWGQGSLVTVS | |
| | | SASTKGPSVFPLAPSSKSTSGGTAA | |
| | | LGCLVKDYFPEPVTVSWNSGALTSG | |
| | | VHTFPAVLQSSG | |
| | HC | QVQLVQSGAEVKKPGSSVKVSCKAS | 808 |
| | variable | GDTFGSYSITWVRQAPGQGLEWMGR | |
| | | IIPVLGIANYAQKFQGRVTITADKS | |
| | | TSTAYMELSSLRSEDTAVYYCAGGG | |
| | | CSGGNCYSWYNWFDPWGQGSLVTVS | |
| | | S | |
| | HCDR1 | SYSIT | 693 |
| | HCDR2 | RIIPVLGIANYAQKFQG | 809 |
| | HCDR3 | GGCSGGNCYSWYNWFDP | 810 |
| | HFR1 | QVQLVQSGAEVKKPGSSVKVSCKAS | 811 |
| | | GDTFG | |
| | HFR2 | WVRQAPGQGLEWMG | 160 |
| | HFR3 | RVTITADKSTSTAYMELSSLRSEDT | 812 |
| | | AVYYCAG | |
| | HFR4 | WGQGSLVTVSS | 813 |
| | LC | EIVLTQSPGTLSLSPGERATLSCRA | 814 |
| | | SQSVSSNYLAWYQQKPGQAPRLLIY | |
| | | GASSRATGIPERFSGSGSGTDFTLT | |
| | | ISRLEPEDFAVYYCQQYGRSPYTFG | |
| | | QGTKLEIKRTVAAPSVFIFPPSDEQ | |
| | | LKSGTASVVCLLNNFYPREAKVQWK | |
| | | VDNALQSGNSQESVTEQDSKDSTYS | |
| | | LSSTLTLSKADYE | |
| | LC | EIVLTQSPGTLSLSPGERATLSCRA | 815 |
| | variable | SQSVSSNYLAWYQQKPGQAPRLLIY | |
| | | GASSRATGIPERFSGSGSGTDFTLT | |
| | | ISRLEPEDFAVYYCQQYGRSPYTFG | |
| | | QGTKLEIK | |
| | LCDR1 | RASQSVSSNYLA | 816 |
| | LCDR2 | GASSRAT | 135 |
| | LCDR3 | QQYGRSPYT | 817 |
| | LFR1 | EIVLTQSPGTLSLSPGERATLSC | 50 |
| | LFR2 | WYQQKPGQAPRLLIY | 182 |
| | LFR3 | GIPERFSGSGSGTDFTLTISRLEPE | 818 |
| | | DFAVYYC | |
| | LFR4 | FGQGTKLEIK | 380 |
| 825S144- | HC | QVQLQESGPGLVKPSETLSLTCTVS | 819 |
| 1299 | | GGSISSYYWSWIRQPPGKGLEWIGY | |
| | | INYRGITNYNPSLKSRVTISVDMSK | |
| | | NQFSLKLSSVTAADTAVYSCARLAV | |
| | | ASRGTVDYWGQGTLVTVSSASTKGP | |
| | | SVFPLAPSSKSTSGGTAALGCLVKD | |
| | | YFPEPVTVSWNSGALTSGVHTFPAV | |
| | | LQSSGLYSLSSVVTVPSSNFGTQTY | |
| | | TCNVDHKPSNTKVD | |
| | HC | QVQLQESGPGLVKPSETLSLTCTVS | 820 |
| | variable | GGSISSYYWSWIRQPPGKGLEWIGY | |
| | | INYRGITNYNPSLKSRVTISVDMSK | |
| | | NQFSLKLSSVTAADTAVYSCARLAV | |
| | | ASRGTVDYWGQGTLVTVSS | |
| | HCDR1 | SYYWS | 56 |
| | HCDR2 | YINYRGITNYNPSLKS | 821 |
| | HCDR3 | LAVASRGTVDY | 822 |
| | HFR1 | QVQLQESGPGLVKPSETLSLTCTVS | 24 |
| | | GGSIS | |
| | HFR2 | WIRQPPGKGLEWIG | 7 |
| | HFR3 | RVTISVDMSKNQFSLKLSSVTAADT | 823 |
| | | AVYSCAR | |
| | HFR4 | WGQGTLVTVSS | 60 |
| | LC | QSVLTQPPSASGTPGQRVTISCSGS | 824 |
| | | SSNIGSNYVYWYQQLPGTAPKLLIY | |
| | | RNNQRPSGVPDRFSGSKSGTSASLA | |
| | | ISGLRSEDEADYYCAAWDDSLSVNV | |
| | | VFGGGTKLTVLGQPKAAPSVTLFPP | |
| | | SSEELQANKATLVCLISDFYPGAVT | |
| | | VAWKADSSPVKAGVETTKPSKQSNN | |
| | | KYAASSYLSLTPEQWKSH | |
| | LC | QSVLTQPPSASGTPGQRVTISCSGS | 825 |
| | variable | SSNIGSNYVYWYQQLPGTAPKLLIY | |
| | | RNNQRPSGVPDRFSGSKSGTSASLA | |
| | | ISGLRSEDEADYYCAAWDDSLSVNV | |
| | | VFGGGTKLTVL | |
| | LCDR1 | SGSSSNIGSNYVY | 403 |
| | LCDR2 | RNNQRPS | 404 |
| | LCDR3 | AAWDDSLSVNVV | 826 |
| | LFR1 | QSVLTQPPSASGTPGQRVTISC | 121 |
| | LFR2 | WYQQLPGTAPKLLIY | 122 |
| | LFR3 | GVPDRFSGSKSGTSASLAISGLRSE | 406 |
| | | DEADYYC | |
| | LFR4 | FGGGTKLTVL | 69 |
| S144- | HC | QVQLVQSGTEVKKPGASVKVSCKAS | 827 |
| 1339 | | GYTFTDYYMHWVRQAPGQGLEWMGR | |
| | | INPTSGGTNYPQKFQGSVTMTRDTS | |
| | | LSTVYMELSGLRSDDTAVYYCARE | |
| | | RVTLIQGKNHYYMDVWGTGTTVTVS | |
| | | SASTKGPSVFPLAPSSKSTSGGTAA | |
| | | LGCLVKDYFPEPVTVSWNSGALTSG | |
| | | VHTFPAVLQSSG | |
| | HC | QVQLVQSGTEVKKPGASVKVSCKAS | 828 |
| | variable | GYTFTDYYMHWVRQAPGQGLEWMGR | |
| | | INPTSGGTNYPQKFQGSVTMTRDTS | |
| | | LSTVYMELSGLRSDDTAVYYCARER | |
| | | VTLIQGKNHYYMDVWGTGTTVTVSS | |
| | HCDR1 | DYYMH | 829 |
| | HCDR2 | RINPTSGGTNYPQKFQG | 830 |
| | HCDR3 | ERVTLIQGKNHYYMDV | 831 |
| | HFR1 | QVQLVQSGTEVKKPGASVKVSCKAS | 832 |
| | | GYTFT | |
| | HFR2 | WVRQAPGQGLEWMG | 160 |
| | HFR3 | SVTMTRDTSLSTVYMELSGLRSDDT | 833 |
| | | AVYYCAR | |
| | HFR4 | WGTGTTVTVSS | 834 |
| | LC | QSALTQPASVSGSPGQSITISCTGT | 835 |
| | | NSDVGGYNYVSWYQQHPGKAPRLMI | |
| | | YDVSNRPSGVSNRFSGSKSGNTASL | |
| | | TISGLQAEDEADYYCSSYTSSSTLV | |
| | | VFGGGTKLTVLGQPKAAPSVTLFPP | |
| | | SSEELQANKATLVCLISDFYPGAVT | |
| | | VAWKADSSPVKAGVETTTPSKQSNN | |
| | | KYAASSYLSLTPEQWKSH | |
| | LC | QSALTQPASVSGSPGQSITISCTGT | 836 |
| | variable | NSDVGGYNYVSWYQQHPGKAPRLMI | |
| | | YDVSNRPSGVSNRFSGSKSGNTASL | |
| | | TISGLQAEDEADYYCSSYTSSSTLV | |
| | | VFGGGTKLTVL | |
| | LCDR1 | TGTNSDVGGYNYVS | 837 |
| | LCDR2 | DVSNRPS | 64 |
| | LCDR3 | SSYTSSSTLVV | 838 |
| | LFR1 | QSALTQPASVSGSPGQSITISC | 66 |
| | LFR2 | WYQQHPGKAPRLMIY | 839 |
| | LFR3 | GVSNRFSGSKSGNTASLTISGLQAE | 68 |
| | | DEADYYC | |
| | LFR4 | FGGGTKLTVL | 69 |
| S144- | HC | QVQLVQSGAEVKKPGASVKVSCKAS | 840 |
| 1406 | | GYTFTTYAMHWVRQAPGQRLEWMGW | |
| | | INAGNGNTKYSQNFQGRVTITRDTS | |
| | | ASTAYMELSSLRSEDTAVYYCASLV | |
| | | GGDSSSWYDYMDVWGKGTTVTVSSA | |
| | | STKGPSVFPLAPCSRSTSESTAALG | |
| | | CLVKDYFPEPVTVSWNSGALTSGVH | |
| | | TFPAVLQSSGLYSLSSVVTVPSSNF | |
| | HC | QVQLVQSGAEVKKPGASVKVSCKAS | 841 |
| | variable | GYTFTTYAMHWVRQAPGQRLEWMGW | |
| | | INAGNGNTKYSQNFQGRVTITRDTS | |
| | | ASTAYMELSSLRSEDTAVYYCASLV | |
| | | GGDSSSWYDYMDVWGKGTTVTVSS | |
| | HCDR1 | TYAMH | 842 |
| | HCDR2 | WINAGNGNTKYSQNFQG | 843 |
| | HCDR3 | LVGGDSSSWYDYMDV | 844 |
| | HFR1 | QVQLVQSGAEVKKPGASVKVSCKAS | 190 |
| | | GYTFT | |
| | HFR2 | WVRQAPGQRLEWMG | 287 |
| | HFR3 | RVTITRDTSASTAYMELSSLRSEDT | 845 |
| | | AVYYCAS | |
| | HFR4 | WGKGTTVTVSS | 670 |
| | LC | DIQMTQSPSTLSASVGDRVTITCRA | 846 |
| | | SQSISSWLAWYQQKPGKAPKLLIYD | |
| | | ASSLESGVPSRFSGSGSGTEFTLTI | |
| | | SSLQPDDFATYYCQQYNSYPWTFGQ | |
| | | GTKVEIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | DIQMTQSPSTLSASVGDRVTITCRA | 847 |
| | variable | SQSISSWLAWYQQKPGKAPKLLIYD | |
| | | ASSLESGVPSRFSGSGSGTEFTLTI | |
| | | SSLQPDDFATYYCQQYNSYPWTFGQ | |
| | | GTKVEIK | |
| | LCDR1 | RASQSISSWLA | 520 |
| | LCDR2 | DASSLES | 387 |
| | LCDR3 | QQYNSYPWT | 646 |
| | LFR1 | DIQMTQSPSTLSASVGDRVTITC | 523 |
| | LFR2 | WYQQKPGKAPKLLIY | 252 |
| | LFR3 | GVPSRFSGSGSGTEFTLTISSLQPD | 524 |
| | | DFATYYC | |
| | LFR4 | FGQGTKVEIK | 53 |
| S144- | HC | QVQLVQSGAEVKKPGSSVKVSCKAS | 848 |
| 1407 | | GGTFSSYTISWVRQAPGQGLEWMGR | |
| | | IIPVRDIANYAQKFQGRVTITADKS | |
| | | TRTAYMEVSSLRSEDTAVYYCAATE | |
| | | LRSDGLDIWGQGTMVTVSSASTKGP | |
| | | SVFPLAPSSKSTSGGTAALGCLVKD | |
| | | YFPEPVTVSWNSGALTSGVHTFPAV | |
| | | LQSSG | |
| | HC | QVQLVQSGAEVKKPGSSVKVSCKAS | 849 |
| | variable | GGTFSSYTISWVRQAPGQGLEWMGR | |
| | | IIPVRDIANYAQKFQGRVTITADKS | |
| | | TRTAYMEVSSLRSEDTAVYYCAATE | |
| | | LRSDGLDIWGQGTMVTVSS | |
| | HCDR1 | SYTIS | 850 |
| | HCDR2 | RIIPVRDIANYAQKFQG | 851 |
| | HCDR3 | TELRSDGLDI | 852 |
| | HFR1 | QVQLVQSGAEVKKPGSSVKVSCKAS | 310 |
| | | GGTFS | |
| | HFR2 | WVRQAPGQGLEWMG | 160 |
| | HFR3 | RVTITADKSTRTAYMEVSSLRSEDT | 853 |
| | | AVYYCAA | |
| | HFR4 | WGQGTMVTVSS | 44 |
| | LC | DIQMTQSPSTLSASVGDRVTITCRA | 854 |
| | | SQSISSWLAWYQQKPGKAPKLLIYD | |
| | | ASSLESGVPSRFSGSGSGTEFTLTV | |
| | | SSLQPDDFATYYCQQYNNYSPITFG | |
| | | QGTKLEIKRTVAAPSVFIFPPSDEQ | |
| | | LKSGTASVVCLLNNFYPREAKVQWK | |
| | | VDNALQSGNSQESVTEQDSKDSTYS | |
| | | LSSTLTLSKADYE | |
| | LC | DIQMTQSPSTLSASVGDRVTITCRA | 855 |
| | variable | SQSISSWLAWYQQKPGKAPKLLIYD | |
| | | ASSLESGVPSRFSGSGSGTEFTLTV | |
| | | SSLQPDDFATYYCQQYNNYSPITFG | |
| | | QGTKLEIK | |
| | LCDR1 | RASQSISSWLA | 520 |
| | LCDR2 | DASSLES | 387 |
| | LCDR3 | QQYNNYSPIT | 856 |
| | LFR1 | DIQMTQSPSTLSASVGDRVTITC | 523 |
| | LFR2 | WYQQKPGKAPKLLIY | 252 |
| | LFR3 | GVPSRFSGSGSGTEFTLTVSSLQPD | 857 |
| | | DFATYYC | |
| | LFR4 | FGQGTKLEIK | 380 |
| S144- | HC | QVQLVQSGAEVKKPGASVKVSCKAS | 858 |
| 1569 | | GYTFSNYGISWVRQAPGQGLEWMGW | |
| | | ISAYNGNTKYPQKLQGRVTMSTDTS | |
| | | TSTAYMELRSLRSDDTAVYYCARET | |
| | | RYGMDVWGQGTTVTVSSASTKGPSV | |
| | | FPLAPSSKSTSGGTAALGCLVKDYF | |
| | | PEPVTVSWNSGALTSGVHTFPAVLQ | |
| | | SSG | |
| | HC | QVQLVQSGAEVKKPGASVKVSCKAS | 859 |
| | variable | GYTFSNYGISWVRQAPGQGLEWMGW | |
| | | ISAYNGNTKYPQKLQGRVTMSTDTS | |
| | | TSTAYMELRSLRSDDTAVYYCARET | |
| | | RYGMDVWGQGTTVTVSS | |
| | HCDR1 | NYGIS | 860 |
| | HCDR2 | WISAYNGNTKYPQKLQG | 861 |
| | HCDR3 | ETRYGMDV | 862 |
| | HFR1 | QVQLVQSGAEVKKPGASVKVSCKAS | 863 |
| | | GYTFS | |
| | HFR2 | WVRQAPGQGLEWMG | 160 |
| | HFR3 | RVTMSTDTSTSTAYMELRSLRSDDT | 864 |
| | | AVYYCAR | |
| | HFR4 | WGQGTTVTVSS | 147 |
| | LC | QPVLTQPPSASASLGASVTLTCTLS | 865 |
| | | SGYSNYKVDWYQQRPGKGPQFVMRV | |
| | | GTGGIVGSKGDGIPDRFSVLGSGLN | |
| | | RYLTIKNIQEEDESDYHCGADHGSG | |
| | | SNFVRVFGGGTKLTVLGQPKAAPSV | |
| | | TLFPPSSEELQANKATLVCLISDFY | |
| | | PGAVTVAWKADSSPVKAGVETTTPS | |
| | | KQSNNKYAASSYLSLTPEQWKSH | |
| | LC | QPVLTQPPSASASLGASVTLTCTLS | 866 |
| | variable | SGYSNYKVDWYQQRPGKGPQFVMRV | |
| | | GTGGIVGSKGDGIPDRFSVLGSGLN | |
| | | RYLTIKNIQEEDESDYHCGADHGSG | |
| | | SNFVRVFGGGTKLTVL | |
| | LCDR1 | TLSSGYSNYKVD | 867 |
| | LCDR2 | VGTGGIVGSKGD | 868 |
| | LCDR3 | GADHGSGSNFVRV | 869 |
| | LFR1 | QPVLTQPPSASASLGASVTLTC | 870 |
| | LFR2 | WYQQRPGKGPQFVMR | 871 |
| | LFR3 | GIPDRFSVLGSGLNRYLTIKNIQEE | 872 |
| | | DESDYHC | |
| | LFR4 | FGGGTKLTVL | 69 |
| S144- | HC | EVQLVQSGAEVKKPGESLKISCKGS | 873 |
| 1641 | | GYTFTSYWIGWVRQMPGKGLEWMGI | |
| | | IYLGDSDTRYSPSFQGQVTISADKS | |
| | | ISTAYLQWNSLKASDTAMYYCARQV | |
| | | TGTTSWFDPWGQGTLVTVSSASTKG | |
| | | PSVFPLAPSSKSTSGGTAALGCLVK | |
| | | DYFPEPVTVSWNSGALTSGVHTFPA | |
| | | VLQSSG | |
| | HC | EVQLVQSGAEVKKPGESLKISCKGS | 874 |
| | variable | GYTFTSYWIGWVRQMPGKGLEWMGI | |
| | | IYLGDSDTRYSPSFQGQVTISADKS | |
| | | ISTAYLQWNSLKASDTAMYYCARQV | |
| | | TGTTSWFDPWGQGTLVTVSS | |
| | HCDR1 | SYWIG | 542 |
| | HCDR2 | IIYLGDSDTRYSPSFQG | 875 |
| | HCDR3 | QVTGTTSWFDP | 876 |
| | HFR1 | EVQLVQSGAEVKKPGESLKISCKGS | 877 |
| | | GYTFT | |
| | HFR2 | WVRQMPGKGLEWMG | 174 |
| | HFR3 | QVTISADKSISTAYLQWNSLKASDT | 878 |
| | | AMYYCAR | |
| | HFR4 | WGQGTLVTVSS | 60 |
| | LC | DIQMTQSPSTLSASVGERVTITCRA | 879 |
| | | SQSISRWLAWYQQKPGKAPKLLIYD | |
| | | ASSLESGVPSRFSGSGSGTEFTLTI | |
| | | SSLQPDDFATYHCHQYSTYSLTFGG | |
| | | GTKVDIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | DIQMTQSPSTLSASVGERVTITCRA | 880 |
| | variable | SQSISRWLAWYQQKPGKAPKLLIYD | |
| | | ASSLESGVPSRFSGSGSGTEFTLTI | |
| | | SSLQPDDFATYHCHQYSTYSLTFGG | |
| | | GTKVDIK | |
| | LCDR1 | RASQSISRWLA | 881 |
| | LCDR2 | DASSLES | 387 |
| | LCDR3 | HQYSTYSLT | 882 |
| | LFR1 | DIQMTQSPSTLSASVGERVTITC | 883 |
| | LFR2 | WYQQKPGKAPKLLIY | 252 |
| | LFR3 | GVPSRFSGSGSGTEFTLTISSLQPD | 884 |
| | | DFATYHC | |
| | LFR4 | FGGGTKVDIK | 885 |
| S144- | HC | EVQLVESGGDVVQPGGSLRLSCAAS | 886 |
| 1827 | | GITFSNYWMTWVRQAPGKGLEWVAT | |
| | | IKKDGGEQYYVDSVKGRFTISRDNA | |
| | | RNSLYLQINSLRAEDTAVYYCARGG | |
| | | SSSSYYWIYWGQGTLVTVSSGSASA | |
| | | PTLFPLVSCENSPSDTSSV | |
| | HC | EVQLVESGGDVVQPGGSLRLSCAAS | 887 |
| | variable | GITFSNYWMTWVRQAPGKGLEWVAT | |
| | | IKKDGGEQYYVDSVKGRFTISRDNA | |
| | | RNSLYLQINSLRAEDTAVYYCARGG | |
| | | SSSSYYWIYWGQGTLVTVSS | |
| | HCDR1 | NYWMT | 888 |
| | HCDR2 | TIKKDGGEQYYVDSVKG | 889 |
| | HCDR3 | GGSSSSYYWIY | 890 |
| | HFR1 | EVQLVESGGDVVQPGGSLRLSCAAS | 891 |
| | | GITFS | |
| | HFR2 | WVRQAPGKGLEWVA | 145 |
| | HFR3 | RFTISRDNARNSLYLQINSLRAEDT | 892 |
| | | AVYYCAR | |
| | HFR4 | WGQGTLVTVSS | 60 |
| | LC | EIVLTQSPGTLSLSPGERATLSCRA | 893 |
| | | SQSISNSYLVWYQQKPGQAPRLLIY | |
| | | GASTRATGIPDRFSGSGSGTDFTLT | |
| | | ISRLEPEDFAVYYCQQYGSSPWTFG | |
| | | QGTTVEIKRTVAAPSVFIFPPSDEQ | |
| | | LKSGTASVVCLLNNFYPREAKVQWK | |
| | | VDNALQSGNSQESVTEQDSKDSTYS | |
| | | LSSTLTLSKADYE | |
| | LC | EIVLTQSPGTLSLSPGERATLSCRA | 894 |
| | variable | SQSISNSYLVWYQQKPGQAPRLLIY | |
| | | GASTRATGIPDRFSGSGSGTDFTLT | |
| | | ISRLEPEDFAVYYCQQYGSSPWTFG | |
| | | QGTTVEIK | |
| | LCDR1 | RASQSISNSYLV | 895 |
| | LCDR2 | GASTRAT | 208 |
| | LCDR3 | QQYGSSPWT | 303 |
| | LFR1 | EIVLTQSPGTLSLSPGERATLSC | 50 |
| | LFR2 | WYQQKPGQAPRLLIY | 182 |
| | LFR3 | GIPDRFSGSGSGTDFTLTISRLEPE | 196 |
| | | DFAVYYC | |
| | LFR4 | FGQGTTVEIK | 896 |
| S144- | HC | EVQLVESGGGLVKPGGSLRLSCAAS | 897 |
| 1848 | | GFTFSSYSMNWVRQAPGKGLEWVSS | |
| | | ISSSSSYIYYADSVKGRFTISRDNA | |
| | | KNSLYLQLNSLRAEDTAVYYCARDR | |
| | | DQLIFSAAFDIWGQGTMVTVSSAST | |
| | | KGPSVFPLAPSSKSTSGGTAALGCL | |
| | | VKDYFPEPVTVSWNSGALTSGVHTF | |
| | | PAVLQSSG | |
| | HC | EVQLVESGGGLVKPGGSLRLSCAAS | 898 |
| | variable | GFTFSSYSMNWVRQAPGKGLEWVSS | |
| | | ISSSSSYIYYADSVKGRFTISRDNA | |
| | | KNSLYLQLNSLRAEDTAVYYCARDR | |
| | | DQLIFSAAFDIWGQGTMVTVSS | |
| | HCDR1 | SYSMN | 126 |
| | HCDR2 | SISSSSSYIYYADSVKG | 899 |
| | HCDR3 | DRDQLIFSAAFDI | 900 |
| | HFR1 | EVQLVESGGGLVKPGGSLRLSCAAS | 602 |
| | | GFTFS | |
| | HFR2 | WVRQAPGKGLEWVS | 130 |
| | HFR3 | RFTISRDNAKNSLYLQLNSLRAEDT | 901 |
| | | AVYYCAR | |
| | HFR4 | WGQGTMVTVSS | 44 |
| | LC | QSVLTQPPSASGTPGQRVTISCSGS | 902 |
| | | SSNIEHNYVFWYQQLPGTAPKLLIY | |
| | | SNNHRPSGVPDRFSGSKSGTSASLA | |
| | | ISGLRSEDEADYYCAAWDASLSGPV | |
| | | VFAGGTKLTVLGQPKAAPSVTLFPP | |
| | | SSEELQANKATLVCLISDFYPGAVT | |
| | | VAWKADSSPVKAGVETTTPSKQSNN | |
| | | KYAASS | |
| | LC | QSVLTQPPSASGTPGQRVTISCSGS | 903 |
| | variable | SSNIEHNYVFWYQQLPGTAPKLLIY | |
| | | SNNHRPSGVPDRFSGSKSGTSASLA | |
| | | ISGLRSEDEADYYCAAWDASLSGPV | |
| | | VFAGGTKLTVL | |
| | LCDR1 | SGSSSNIEHNYVF | 904 |
| | LCDR2 | SNNHRPS | 905 |
| | LCDR3 | AAWDASLSGPVV | 906 |
| | LFR1 | QSVLTQPPSASGTPGQRVTISC | 121 |
| | LFR2 | WYQQLPGTAPKLLIY | 122 |
| | LFR3 | GVPDRFSGSKSGTSASLAISGLRSE | 406 |
| | | DEADYYC | |
| | LFR4 | FAGGTKLTVL | 907 |
| S144- | HC | EVQLVESGGGLVQPGGSLRLSCAAS | 908 |
| 1850 | | GFTFSSYAMSWVRQAPGKGLEWVSA | |
| | | ISGSGGSTYYADSVKGRFTISRANS | |
| | | KNTLYLQMNSLRAEDTAVYYCAKGP | |
| | | RFSRDYFDYWGQGTLVTVSSASTKG | |
| | | PSVFPLAPSSKSTSGGTAALGCLVK | |
| | | DYFPEPVTVSWNSGALTSGVHTFPA | |
| | | VLQSSG | |
| | HC | EVQLVESGGGLVQPGGSLRLSCAAS | 909 |
| | variable | GFTFSSYAMSWVRQAPGKGLEWVSA | |
| | | ISGSGGSTYYADSVKGRFTISRANS | |
| | | KNTLYLQMNSLRAEDTAVYYCAKGP | |
| | | RFSRDYFDYWGQGTLVTVSS | |
| | HCDR1 | SYAMS | 610 |
| | HCDR2 | AISGSGGSTYYADSVKG | 910 |
| | HCDR3 | GPRFSRDYFDY | 911 |
| | HFR1 | EVQLVESGGGLVQPGGSLRLSCAAS | 613 |
| | | GFTFS | |
| | HFR2 | WVRQAPGKGLEWVS | 130 |
| | HFR3 | RFTISRANSKNTLYLQMNSLRAEDT | 912 |
| | | AVYYCAK | |
| | HFR4 | WGQGTLVTVSS | 60 |
| | LC | DIQMTQSPSTLSASVGDRVTITCRA | 913 |
| | | SQSITSWLAWYQQKPGKAPKLLIYD | |
| | | ASNLESGVPSRFSGSGSGTEFTLTI | |
| | | SSLQPDDFATYYCQQYNNYLGTFGQ | |
| | | GTKVEIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | DIQMTQSPSTLSASVGDRVTITCRA | 914 |
| | variable | SQSITSWLAWYQQKPGKAPKLLIYD | |
| | | ASNLESGVPSRFSGSGSGTEFTLTI | |
| | | SSLQPDDFATYYCQQYNNYLGTFGQ | |
| | | GTKVEIK | |
| | LCDR1 | RASQSITSWLA | 645 |
| | LCDR2 | DASNLES | 915 |
| | LCDR3 | QQYNNYLGT | 916 |
| | LFR1 | DIQMTQSPSTLSASVGDRVTITC | 523 |
| | LFR2 | WYQQKPGKAPKLLIY | 252 |
| | LFR3 | GVPSRFSGSGSGTEFTLTISSLQPD | 524 |
| | | DFATYYC | |
| | LFR4 | FGQGTKVEIK | 53 |
| S144- | HC | QVQLVQSGAEVKKPGSSVKVSCKAS | 917 |
| 2234 | | GGTFSRYTISWVRQAPGQGLEWMGR | |
| | | IIPILGTANYAQNFQGRVTITADKS | |
| | | TSTAYMELSSLRSEDTAVYYCARHG | |
| | | YSYGPFDYWGQGTLVTVSSASTKGP | |
| | | SVFPLAPSSKSTSGGTAALGCLVKD | |
| | | YFPEPVTVSWNSGALTSGVHTFPAV | |
| | | LQSSG | |
| | HC | QVQLVQSGAEVKKPGSSVKVSCKAS | 918 |
| | variable | GGTFSRYTISWVRQAPGQGLEWMGR | |
| | | IIPILGTANYAQNFQGRVTITADKS | |
| | | TSTAYMELSSLRSEDTAVYYCARHG | |
| | | YSYGPFDYWGQGTLVTVSS | |
| | HCDR1 | RYTIS | 919 |
| | HCDR2 | RIIPILGTANYAQNFQG | 920 |
| | HCDR3 | HGYSYGPFDY | 921 |
| | HFR1 | QVQLVQSGAEVKKPGSSVKVSCKAS | 310 |
| | | GGTFS | |
| | HFR2 | WVRQAPGQGLEWMG | 160 |
| | HFR3 | RVTITADKSTSTAYMELSSLRSEDT | 161 |
| | | AVYYCAR | |
| | HFR4 | WGQGTLVTVSS | 60 |
| | LC | DIVMTQSPDSLTVSLGERATINCKS | 922 |
| | | SQSVLYSSNNKNYLAWYQQKPGQPP | |
| | | KLLIYWASTRESGVPDRFSGSGSGT | |
| | | DFTLTVSSLQAEDVAVYYCQQYYST | |
| | | PGTFGQGTKVEIKRTVAAPSVFIFP | |
| | | PSDEQLKSGTASVVCLLNNFYPREA | |
| | | KVQWKVDNALQSGNSQESVTEQDSK | |
| | | DSTYSLSSTLTLSKADYE | |
| | LC | DIVMTQSPDSLTVSLGERATINCKS | 923 |
| | variable | SQSVLYSSNNKNYLAWYQQKPGQPP | |
| | | KLLIYWASTRESGVPDRFSGSGSGT | |
| | | DFTLTVSSLQAEDVAVYYCQQYYST | |
| | | PGTFGQGTKVEIK | |
| | LCDR1 | KSSQSVLYSSNNKNYLA | 291 |
| | LCDR2 | WASTRES | 30 |
| | LCDR3 | QQYYSTPGT | 924 |
| | LFR1 | DIVMTQSPDSLTVSLGERATINC | 925 |
| | LFR2 | WYQQKPGQPPKLLIY | 33 |
| | LFR3 | GVPDRFSGSGSGTDFTLTVSSLQAE | 926 |
| | | DVAVYYC | |
| | LFR4 | FGQGTKVEIK | 53 |
| S564-105 | HC | QVRLQESGPGLVKPSQTLSLTCTVS | 927 |
| | | GGSISSGSYYWSWIRQPAGKGLEWI | |
| | | GRFHTSGSTNYNPSLKSRVTISVDT | |
| | | SKNQFSLKLSSVTAADTAVYYCARD | |
| | | LKGKTWIQTPFDYWGQGILVTVSSA | |
| | | STKGPSVFPLAPSSKSTSGGTAALG | |
| | | CLVKDYFPEPVTVSWNSGALTSGVH | |
| | | TFPAVLQSSG | |
| | HC | QVRLQESGPGLVKPSQTLSLTCTVS | 928 |
| | variable | GGSISSGSYYWSWIRQPAGKGLEWI | |
| | | GRFHTSGSTNYNPSLKSRVTISVDT | |
| | | SKNQFSLKLSSVTAADTAVYYCARD | |
| | | LKGKTWIQTPFDYWGQGILVTVSS | |
| | HCDR1 | SGSYYWS | 929 |
| | HCDR2 | RFHTSGSTNYNPSLKS | 930 |
| | HCDR3 | DLKGKTWIQTPFDY | 931 |
| | HFR1 | QVRLQESGPGLVKPSQTLSLTCTVS | 932 |
| | | GGSIS | |
| | HFR2 | WIRQPAGKGLEWIG | 25 |
| | HFR3 | RVTISVDTSKNQFSLKLSSVTAADT | 115 |
| | | AVYYCAR | |
| | HFR4 | WGQGILVTVSS | 220 |
| | LC | QSALTQPASVSGSPGQSITISCTGT | 933 |
| | | SSDVGAYNYVSWYQQHPGKAPKLMI | |
| | | YEVSNRPSGVSNRFSGSKSGNTASL | |
| | | TISGLQAEDEADYYCSSYTSSTFFG | |
| | | TGTTVTVLGQPKANPTVTLFPPSSE | |
| | | ELQANKATLVCLISDFYPGAVTVAW | |
| | | KADGSPVKAGVETTTPSKQSNNKYA | |
| | | ASSY | |
| | LC | QSALTQPASVSGSPGQSITISCTGT | 934 |
| | variable | SSDVGAYNYVSWYQQHPGKAPKLMI | |
| | | YEVSNRPSGVSNRFSGSKSGNTASL | |
| | | TISGLQAEDEADYYCSSYTSSTFFG | |
| | | TGTTVTVL | |
| | LCDR1 | TGTSSDVGAYNYVS | 935 |
| | LCDR2 | EVSNRPS | 151 |
| | LCDR3 | SSYTSSTF | 936 |
| | LFR1 | QSALTQPASVSGSPGQSITISC | 66 |
| | LFR2 | WYQQHPGKAPKLMIY | 67 |
| | LFR3 | GVSNRFSGSKSGNTASLTISGLQAE | 68 |
| | | DEADYYC | |
| | LFR4 | FGTGTTVTVL | 937 |
| S564-14 | HC | EVQLVESGGGLVQPGGSLRLSCAAS | 938 |
| | | GLTFSSYWMSWARQAPGKGLEWVAN | |
| | | IKKDGSEKYYVDSVKGRFTISRDNA | |
| | | KNSLYLQMNSLRVEDTAVYYCASEP | |
| | | PHYGGNSGAEYFQHWGQGTLVTVSS | |
| | | APTKAPDVFPIISGCRHPKDNSPVV | |
| | | LACLITGYH | |
| | HC | EVQLVESGGGLVQPGGSLRLSCAAS | 939 |
| | variable | GLTFSSYWMSWARQAPGKGLEWVAN | |
| | | IKKDGSEKYYVDSVKGRFTISRDNA | |
| | | KNSLYLQMNSLRVEDTAVYYCASEP | |
| | | PHYGGNSGAEYFQHWGQGTLVTVSS | |
| | HCDR1 | SYWMS | 270 |
| | HCDR2 | NIKKDGSEKYYVDSVKG | 940 |
| | HCDR3 | EPPHYGGNSGAEYFQH | 941 |
| | HFR1 | EVQLVESGGGLVQPGGSLRLSCAAS | 942 |
| | | GLTFS | |
| | HFR2 | WARQAPGKGLEWVA | 943 |
| | HFR3 | RFTISRDNAKNSLYLQMNSLRVEDT | 944 |
| | | AVYYCAS | |
| | HFR4 | WGQGTLVTVSS | 60 |
| | LC | SYVLTQPPSVSVAPGKTARITCGGN | 945 |
| | | NIGSKSVHWYQQRPGQAPVLVIYYD | |
| | | SDRPSGIPERFSGSNSGNTATLTIS | |
| | | RVEAGDEADYYCQVWDSSSDHHYVF | |
| | | GTGTKVTVLGQPKANPTVTLFPPSS | |
| | | EELQANKATLVCLISDFYPGAVTVA | |
| | | WKADSSPVKAGVETTKPSKQSNNKY | |
| | | AASS | |
| | LC | SYVLTQPPSVSVAPGKTARITCGGN | 946 |
| | variable | NIGSKSVHWYQQRPGQAPVLVIYYD | |
| | | SDRPSGIPERFSGSNSGNTATLTIS | |
| | | RVEAGDEADYYCQVWDSSSDHHYVF | |
| | | GTGTKVTVL | |
| | LCDR1 | GGNNIGSKSVH | 12 |
| | LCDR2 | YDSDRPS | 947 |
| | LCDR3 | QVWDSSSDHHYV | 948 |
| | LFR1 | SYVLTQPPSVSVAPGKTARITC | 949 |
| | LFR2 | WYQQRPGQAPVLVIY | 950 |
| | LFR3 | GIPERFSGSNSGNTATLTISRVEAG | 17 |
| | | DEADYYC | |
| | LFR4 | FGTGTKVTVL | 18 |
| S564-68 | HC | QVQLVQSGAEVKKPGASVKVSCKAS | 951 |
| | | GYIFTGYYMHWVRQAPGQGLEWMGW | |
| | | INPNSGGTNYAQKFQGRVTMTRDTS | |
| | | ITTAYMELSRLRSDDTAFYYCARVK | |
| | | RFSIFGVELDYWGQGTLVTVSSAST | |
| | | KGPSVFPLAPSSKSTSGGTAALGCL | |
| | | VKDYFPEPVTVSWNSGALTSGVHTF | |
| | | PAVLQSSG | |
| | HC | QVQLVQSGAEVKKPGASVKVSCKAS | 952 |
| | variable | GYIFTGYYMHWVRQAPGQGLEWMGW | |
| | | INPNSGGTNYAQKFQGRVTMTRDTS | |
| | | ITTAYMELSRLRSDDTAFYYCARVK | |
| | | RFSIFGVELDYWGQGTLVTVSS | |
| | HCDR1 | GYYMH | 187 |
| | HCDR2 | WINPNSGGTNYAQKFQG | 953 |
| | HCDR3 | VKRFSIFGVELDY | 954 |
| | HFR1 | QVQLVQSGAEVKKPGASVKVSCKAS | 955 |
| | | GYIFT | |
| | HFR2 | WVRQAPGQGLEWMG | 160 |
| | HFR3 | RVTMTRDTSITTAYMELSRLRSDDT | 956 |
| | | AFYYCAR | |
| | HFR4 | WGQGTLVTVSS | 60 |
| | LC | QSALTQPPSASGSPGQSVTISCTGT | 957 |
| | | SSDVGGYNYVSWYQQHPGKAPKLMI | |
| | | YEVSKRPSGVPDRFSGSKSGNTASL | |
| | | TVSGLQAEDEADYFCSSYADSNNLV | |
| | | FGGGTKLTVLGQPKAAPSVTLFPPS | |
| | | SEELQANKATLVCLISDFCPGAVTV | |
| | | AWKADSSPVKAGVETTTPSKQSNNK | |
| | | YAASSY | |
| | LC | QSALTQPPSASGSPGQSVTISCTGT | 958 |
| | variable | SSDVGGYNYVSWYQQHPGKAPKLMI | |
| | | YEVSKRPSGVPDRFSGSKSGNTASL | |
| | | TVSGLQAEDEADYFCSSYADSNNLV | |
| | | FGGGTKLTVL | |
| | LCDR1 | TGTSSDVGGYNYVS | 63 |
| | LCDR2 | EVSKRPS | 94 |
| | LCDR3 | SSYADSNNLV | 959 |
| | LFR1 | QSALTQPPSASGSPGQSVTISC | 96 |
| | LFR2 | WYQQHPGKAPKLMIY | 67 |
| | LFR3 | GVPDRFSGSKSGNTASLTVSGLQAE | 960 |
| | | DEADYFC | |
| | LFR4 | FGGGTKLTVL | 69 |
| S564-98 | HC | QVQLQESGPGLVKPSETLSLTCTVS | 961 |
| | | GGSISSYYWSWIRQPPGKGLEWIGY | |
| | | IYYSGSTNYNPSLKSRVTISVDTSK | |
| | | NQFSLKLSSVTAADTAVYYCARHQS | |
| | | RWNIVATMDFDYWGQGTLVTVSSAS | |
| | | TKGPSVFPL | |
| | HC | QVQLQESGPGLVKPSETLSLTCTVS | 962 |
| | variable | GGSISSYYWSWIRQPPGKGLEWIGY | |
| | | IYYSGSTNYNPSLKSRVTISVDTSK | |
| | | NQFSLKLSSVTAADTAVYYCARHQS | |
| | | RWNIVATMDFDYWGQGTLVTVSS | |
| | HCDR1 | SYYWS | 56 |
| | HCDR2 | YIYYSGSTNYNPSLKS | 4 |
| | HCDR3 | HQSRWNIVATMDFDY | 963 |
| | HFR1 | QVQLQESGPGLVKPSETLSLTCTVS | 24 |
| | | GGSIS | |
| | HFR2 | WIRQPPGKGLEWIG | 7 |
| | HFR3 | RVTISVDTSKNQFSLKLSSVTAADT | 115 |
| | | AVYYCAR | |
| | HFR4 | WGQGTLVTVSS | 60 |
| | LC | DIQMTQSPSSLSASVGDRVTITCRA | 964 |
| | | SQSIRSYLNWYQQKPGKAPKLLIYA | |
| | | ASSLQSGVPSRFSGSGSGTDFTLTI | |
| | | GSLQPEDFATYYCQQSYSTSVAFGQ | |
| | | GTKVEIKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYE | |
| | LC | DIQMTQSPSSLSASVGDRVTITCRA | 965 |
| | variable | SQSIRSYLNWYQQKPGKAPKLLIYA | |
| | | ASSLQSGVPSRFSGSGSGTDFTLTI | |
| | | GSLQPEDFATYYCQQSYSTSVAFGQ | |
| | | GTKVEIK | |
| | LCDR1 | RASQSIRSYLN | 432 |
| | LCDR2 | AASSLQS | 249 |
| | LCDR3 | QQSYSTSVA | 966 |
| | LFR1 | DIQMTQSPSSLSASVGDRVTITC | 251 |
| | LFR2 | WYQQKPGKAPKLLIY | 252 |
| | LFR3 | GVPSRFSGSGSGTDFTLTIGSLQPE | 967 |
| | | DFATYYC | |
| | LFR4 | FGQGTKVEIK | 53 |
| S564-105 | HC | QVRLQESGPGLVKPSQTLSLTCTVS | 927 |
| | | GGSISSGSYYWSWIRQPAGKGLEWI | |
| | | GRFHTSGSTNYNPSLKSRVTISVDT | |
| | | SKNQFSLKLSSVTAADTAVYYCARD | |
| | | LKGKTWIQTPFDYWGQGILVTVSSA | |
| | | STKGPSVFPLAPSSKSTSGGTAALG | |
| | | CLVKDYFPEPVTVSWNSGALTSGVH | |
| | | TFPAVLQSSG | |
| | HC | QVRLQESGPGLVKPSQTLSLTCTVS | 928 |
| | variable | GGSISSGSYYWSWIRQPAGKGLEWI | |
| | | GRFHTSGSTNYNPSLKSRVTISVDT | |
| | | SKNQFSLKLSSVTAADTAVYYCARD | |
| | | LKGKTWIQTPFDYWGQGILVTVSS | |
| | HCDR1 | SGSYYWS | 929 |
| | HCDR2 | RFHTSGSTNYNPSLKS | 930 |
| | HCDR3 | DLKGKTWIQTPFDY | 931 |
| | HFR1 | QVRLQESGPGLVKPSQTLSLTCTVS | 932 |
| | | GGSIS | |
| | HFR2 | WIRQPAGKGLEWIG | 25 |
| | HFR3 | RVTISVDTSKNQFSLKLSSVTAADT | 115 |
| | | AVYYCAR | |
| | HFR4 | WGQGILVTVSS | 220 |
| | LC | QSALTQPASVSGSPGQSITISCTGT | 933 |
| | | SSDVGAYNYVSWYQQHPGKAPKLMI | |
| | | YEVSNRPSGVSNRFSGSKSGNTASL | |
| | | TISGLQAEDEADYYCSSYTSSTFFG | |
| | | TGTTVTVLGQPKANPTVTLFPPSSE | |
| | | ELQANKATLVCLISDFYPGAVTVAW | |
| | | KADGSPVKAGVETTTPSKQSNNKYA | |
| | | ASSY | |
| | LC | QSALTQPASVSGSPGQSITISCTGT | 934 |
| | variable | SSDVGAYNYVSWYQQHPGKAPKLMI | |
| | | YEVSNRPSGVSNRFSGSKSGNTASL | |
| | | TISGLQAEDEADYYCSSYTSSTFFG | |
| | | TGTTVTVL | |
| | LCDR1 | TGTSSDVGAYNYVS | 935 |
| | LCDR2 | EVSNRPS | 151 |
| | LCDR3 | SSYTSSTF | 936 |
| | LFR1 | QSALTQPASVSGSPGQSITISC | 66 |
| | LFR2 | WYQQHPGKAPKLMIY | 67 |
| | LFR3 | GVSNRFSGSKSGNTASLTISGLQAE | 68 |
| | | DEADYYC | |
| | LFR4 | FGTGTTVTVL | 937 |
| S564-134 | HC | QVQLVQSGAEVKKPGASVKVSCKAS | 968 |
| | | GYTFTGYYMHWVRQAPGQGLEWMGW | |
| | | INPNSGGTNYAQKFQGRVTMTRDTS | |
| | | INTAYMELSRLRSDDTAVYYCTRVG | |
| | | RFSIFGVELDYWGQGTLVTVSSAST | |
| | | KGPSVFPLAPSSKSTSGGTAALGCL | |
| | | VKDYFPEPVTVSWNSGALTSGVHTF | |
| | | PAVLQSSG | |
| | HC | QVQLVQSGAEVKKPGASVKVSCKAS | 969 |
| | variable | GYTFTGYYMHWVRQAPGQGLEWMGW | |
| | | INPNSGGTNYAQKFQGRVTMTRDTS | |
| | | INTAYMELSRLRSDDTAVYYCTRVG | |
| | | RFSIFGVELDYWGQGTLVTVSS | |
| | HCDR1 | GYYMH | 187 |
| | HCDR2 | WINPNSGGTNYAQKFQG | 953 |
| | HCDR3 | VGRFSIFGVELDY | 970 |
| | HFR1 | QVQLVQSGAEVKKPGASVKVSCKAS | 190 |
| | | GYTFT | |
| | HFR2 | WVRQAPGQGLEWMG | 160 |
| | HFR3 | RVTMTRDTSINTAYMELSRLRSDDT | 971 |
| | | AVYYCTR | |
| | HFR4 | WGQGTLVTVSS | 60 |
| | LC | QSALTQPPSASGSPGQSVTISCTGT | 972 |
| | | SSDVGGYNYVSWYQQHPGKAPKLMI | |
| | | YEVNKRPSGVPDRFSGSKSGNTASL | |
| | | TVSGLQADDEADYYCSSYAGSNNLV | |
| | | FGGGTKLTVLGQPKAAPSVTLFPPS | |
| | | SEELQANKATLVCLISDFYPGAVTV | |
| | | AWKADSSPVKAGVETTTPSKQSNNK | |
| | | YAASS | |
| | LC | QSALTQPPSASGSPGQSVTISCTGT | 973 |
| | variable | SSDVGGYNYVSWYQQHPGKAPKLMI | |
| | | YEVNKRPSGVPDRFSGSKSGNTASL | |
| | | TVSGLQADDEADYYCSSYAGSNNLV | |
| | | FGGGTKLTVL | |
| | LCDR1 | TGTSSDVGGYNYVS | 63 |
| | LCDR2 | EVNKRPS | 974 |
| | LCDR3 | SSYAGSNNLV | 975 |
| | LFR1 | QSALTQPPSASGSPGQSVTISC | 96 |
| | LFR2 | WYQQHPGKAPKLMIY | 67 |
| | LFR3 | GVPDRFSGSKSGNTASLTVSGLQAD | 976 |
| | | DEADYYC | |
| | LFR4 | FGGGTKLTVL | 69 |
| S564-138 | HC | QVLLVQSGAEVKKPGASVKVSCKAS | 977 |
| | | GYTFTGYYLHWVRQAPGQGLEWMGW | |
| | | INPISGGTNYAQNFQDRVTMTRDTS | |
| | | IITAYMELSRLRSDDTAVYYCARLA | |
| | | YYYDSSAYRGAFDIWGQGTMVTVSS | |
| | | ASTKGPSVFPLAPSSKSTSGGTAAL | |
| | | GCLVKDYFPEPVTVSWNSGALTSGV | |
| | | HTFPAVLQSS | |
| | HC | QVLLVQSGAEVKKPGASVKVSCKAS | 978 |
| | variable | GYTFTGYYLHWVRQAPGQGLEWMGW | |
| | | INPISGGTNYAQNFQDRVTMTRDTS | |
| | | IITAYMELSRLRSDDTAVYYCARLA | |
| | | YYYDSSAYRGAFDIWGQGTMVTVSS | |
| | HCDR1 | GYYLH | 979 |
| | HCDR2 | WINPISGGTNYAQNFQD | 980 |
| | HCDR3 | LAYYYDSSAYRGAFDI | 981 |
| | HFR1 | QVLLVQSGAEVKKPGASVKVSCKAS | 982 |
| | | GYTFT | |
| | HFR2 | WVRQAPGQGLEWMG | 160 |
| | HFR3 | RVTMTRDTSIITAYMELSRLRSDDT | 983 |
| | | AVYYCAR | |
| | HFR4 | WGQGTMVTVSS | 44 |
| | LC | QSALTQPASVSGSPGQSITISCTGT | 984 |
| | | SSDVGGYNYVSWYQQHPGKAPKLMI | |
| | | YEVSNRPSGVSDRFSGSKSGNTASL | |
| | | TISGLQAEDEADYYCSSYTSSSTYV | |
| | | FGTGTKVTVLGQPKANPTVTLFPPS | |
| | | SEELQANKATLVCLISDFYPGAVTV | |
| | | AWKADGSPVKAGVETTKPSKQSNNK | |
| | | YAASS | |
| | LC | QSALTQPASVSGSPGQSITISCTGT | 985 |
| | variable | SSDVGGYNYVSWYQQHPGKAPKLMI | |
| | | YEVSNRPSGVSDRFSGSKSGNTASL | |
| | | TISGLQAEDEADYYCSSYTSSSTYV | |
| | | FGTGTKVTVL | |
| | LCDR1 | TGTSSDVGGYNYVS | 63 |
| | LCDR2 | EVSNRPS | 151 |
| | LCDR3 | SSYTSSSTYV | 986 |
| | LFR1 | QSALTQPASVSGSPGQSITISC | 66 |
| | LFR2 | WYQQHPGKAPKLMIY | 67 |
| | LFR3 | GVSDRFSGSKSGNTASLTISGLQAE | 987 |
| | | DEADYYC | |
| | LFR4 | FGTGTKVTVL | 18 |
| S564-152 | HC | QVQLVESGGGVVQPGRSLRLSCAAS | 988 |
| | | GFTFSYYGMHWVRQAPGKGLEWVAV | |
| | | IWYDGSNKHYADSVKGRFTISRDNS | |
| | | KNTLYLQMNSLRAEDTAVYYCAKNA | |
| | | APYCSGGSCYGTYFDYWGQGTLVTV | |
| | | SSASTKGPSVFPLAPSSKSTSGGTA | |
| | | ALGCLVKDYFPEPVTVSWNSGALTS | |
| | | GVHTFPAVLQSSG | |
| | HC | QVQLVESGGGVVQPGRSLRLSCAAS | 989 |
| | variable | GFTFSYYGMHWVRQAPGKGLEWVAV | |
| | | IWYDGSNKHYADSVKGRFTISRDNS | |
| | | KNTLYLQMNSLRAEDTAVYYCAKNA | |
| | | APYCSGGSCYGTYFDYWGQGTLVTV | |
| | | SS | |
| | HCDR1 | YYGMH | 990 |
| | HCDR2 | VIWYDGSNKHYADSVKG | 991 |
| | HCDR3 | NAAPYCSGGSCYGTYFDY | 992 |
| | HFR1 | QVQLVESGGGVVQPGRSLRLSCAAS | 144 |
| | | GFTFS | |
| | HFR2 | WVRQAPGKGLEWVA | 145 |
| | HFR3 | RFTISRDNSKNTLYLQMNSLRAEDT | 499 |
| | | AVYYCAK | |
| | HFR4 | WGQGTLVTVSS | 60 |
| | LC | DIQMTQSPSSLSASVGDRVTITCQA | 993 |
| | | SQDINNYLNWYQQKPGKAPKLLIYD | |
| | | ASNLETGVPSRFSGSGSGTDFTFTI | |
| | | SSLQPEDIATYYCQQYDNVPPHTFG | |
| | | QGTKLEIKRTVAAPSVFIFPPSDEQ | |
| | | LKSGTASVVCLLNNFYPREAKVQWK | |
| | | VDNALQSGNSQESVTEQDSKDSTYS | |
| | | LSSTLTLSKADYE | |
| | LC | DIQMTQSPSSLSASVGDRVTITCQA | 994 |
| | variable | SQDINNYLNWYQQKPGKAPKLLIYD | |
| | | ASNLETGVPSRFSGSGSGTDFTFTI | |
| | | SSLQPEDIATYYCQQYDNVPPHTFG | |
| | | QGTKLEIK | |
| | LCDR1 | QASQDINNYLN | 995 |
| | LCDR2 | DASNLET | 770 |
| | LCDR3 | QQYDNVPPHT | 996 |
| | LFR1 | DIQMTQSPSSLSASVGDRVTITC | 251 |
| | LFR2 | WYQQKPGKAPKLLIY | 252 |
| | LFR3 | GVPSRFSGSGSGTDFTFTISSLQPE | 997 |
| | | DIATYYC | |
| | LFR4 | FGQGTKLEIK | 380 |
| S564-218 | HC | QVQLVQSGAEVKKPGSSVKVSCKAS | 998 |
| | | GGTFSSYAISWVRQAPGQGLEWMGG | |
| | | IIPIFGTAKYAQKFQGRVTITADES | |
| | | TSTAYMELSSLRSEDTAVYYCARGK | |
| | | DGYNPWGAFDIWGQGTMVTVSSGSA | |
| | | SAPTLFPLVSCENSPSDTSSV | |
| | HC | QVQLVQSGAEVKKPGSSVKVSCKAS | 999 |
| | variable | GGTFSSYAISWVRQAPGQGLEWMGG | |
| | | IIPIFGTAKYAQKFQGRVTITADES | |
| | | TSTAYMELSSLRSEDTAVYYCARGK | |
| | | DGYNPWGAFDIWGQGTMVTVSS | |
| | HCDR1 | SYAIS | 308 |
| | HCDR2 | GIIPIFGTAKYAQKFQG | 1000 |
| | HCDR3 | GKDGYNPWGAFDI | 1001 |
| | HFR1 | QVQLVQSGAEVKKPGSSVKVSCKAS | 310 |
| | | GGTFS | |
| | HFR2 | WVRQAPGQGLEWMG | 160 |
| | HFR3 | RVTITADESTSTAYMELSSLRSEDT | 1002 |
| | | AVYYCAR | |
| | HFR4 | WGQGTMVTVSS | 44 |
| | LC | QSALTQPPSASGSPGQSVTISCTGT | 1003 |
| | | SSDVGGYNYVSWYQQHPGKAPKLMI | |
| | | YEVSKRPSGVPDRFSGSKSGNTASL | |
| | | TVSGLQAEDEADYYCSSYAGSNNFG | |
| | | VFGGGTKLTVLGQPKAAPSVTLFPP | |
| | | SSEELQANKATLVCLISDFYPGAVT | |
| | | VAWKADSSPVKAGVETTTPSKQSNN | |
| | | KYAASSYLSLTPEQWKSH | |
| | LC | QSALTQPPSASGSPGQSVTISCTGT | 1004 |
| | variable | SSDVGGYNYVSWYQQHPGKAPKLMI | |
| | | YEVSKRPSGVPDRFSGSKSGNTASL | |
| | | TVSGLQAEDEADYYCSSYAGSNNFG | |
| | | VFGGGTKLTVL | |
| | LCDR1 | TGTSSDVGGYNYVS | 63 |
| | LCDR2 | EVSKRPS | 94 |
| | LCDR3 | SSYAGSNNFGV | 1005 |
| | LFR1 | QSALTQPPSASGSPGQSVTISC | 96 |
| | LFR2 | WYQQHPGKAPKLMIY | 67 |
| | LFR3 | GVPDRFSGSKSGNTASLTVSGLQAE | 1006 |
| | | DEADYYC | |
| | LFR4 | FGGGTKLTVL | 69 |
| S564-249 | HC | EVQLVESGGGLVQPGGSLRLSCVAS | 1007 |
| | | GFTFSDYAMHWVRQAPGKGLEYIAA | |
| | | ISSNGGRTYYADSVKDKFTISRDNS | |
| | | KNILYLHMGSLRAEDTAVYFCARDP | |
| | | QSWVTSTTAHFQHWGQGTLVTVSSA | |
| | | SPTSPKVFPLSLCSTQPDGNVVIAC | |
| | | LVQGFFPQEPLSVTWSESGQGVTAR | |
| | | NF | |
| | HC | EVQLVESGGGLVQPGGSLRLSCVAS | 1008 |
| | variable | GFTFSDYAMHWVRQAPGKGLEYIAA | |
| | | ISSNGGRTYYADSVKDKFTISRDNS | |
| | | KNILYLHMGSLRAEDTAVYFCARDP | |
| | | QSWVTSTTAHFQHWGQGTLVTVSS | |
| | HCDR1 | DYAMH | 1009 |
| | HCDR2 | AISSNGGRTYYADSVKD | 1010 |
| | HCDR3 | DPQSWVTSTTAHFQH | 1011 |
| | HFR1 | EVQLVESGGGLVQPGGSLRLSCVAS | 1012 |
| | | GFTFS | |
| | HFR2 | WVRQAPGKGLEYIA | 1013 |
| | HFR3 | KFTISRDNSKNILYLHMGSLRAEDT | 1014 |
| | | AVYFCAR | |
| | HFR4 | WGQGTLVTVSS | 60 |
| | LC | QSALTQPASVSGSPGQSITISCTGT | 1015 |
| | | SSDIGGYNYVSWYQQHPGKAPKLII | |
| | | SDVSNRPSGVSSRFSGSKSGNTASL | |
| | | TISGLQTEDEAHYYCSSFRSGITLG | |
| | | VFGGGTKLTVLGQPKAAPSVTLFPP | |
| | | SSEELQANKATLVCLISDFYPGAVT | |
| | | VAWKADSSPVKAGVETTTPSKQSNN | |
| | | KYAASS | |
| | LC | QSALTQPASVSGSPGQSITISCTGT | 1016 |
| | variable | SSDIGGYNYVSWYQQHPGKAPKLII | |
| | | SDVSNRPSGVSSRFSGSKSGNTASL | |
| | | TISGLQTEDEAHYYCSSFRSGITLG | |
| | | VFGGGTKLTVL | |
| | LCDR1 | TGTSSDIGGYNYVS | 1017 |
| | LCDR2 | DVSNRPS | 64 |
| | LCDR3 | SSFRSGITLGV | 1018 |
| | LFR1 | QSALTQPASVSGSPGQSITISC | 66 |
| | LFR2 | WYQQHPGKAPKLIIS | 1019 |
| | LFR3 | GVSSRFSGSKSGNTASLTISGLQTE | 1020 |
| | | DEAHYYC | |
| | LFR4 | FGGGTKLTVL | 69 |
| S564-265 | HC | QVQLVQSGAEVKKPGASVKVSCKAS | 1021 |
| | | GYTFTGYYMHWVRQAPGQGLEWMGW | |
| | | INPNSGAINYAQKFQGRVTMTRDTS | |
| | | ISTAYMELSSLRSDDTAVYYCARVG | |
| | | RFSIFGVELDNWGQGTLVTVSSAST | |
| | | KGPSVFPLAPSSKSTSGGTAALGCL | |
| | | VKDYFPEPVTVSWNSGALTSGVHTF | |
| | | PAVLQSSG | |
| | HC | QVQLVQSGAEVKKPGASVKVSCKAS | 1022 |
| | variable | GYTFTGYYMHWVRQAPGQGLEWMGW | |
| | | INPNSGAINYAQKFQGRVTMTRDTS | |
| | | ISTAYMELSSLRSDDTAVYYCARVG | |
| | | RFSIFGVELDNWGQGTLVTVSS | |
| | HCDR1 | GYYMH | 187 |
| | HCDR2 | WINPNSGAINYAQKFQG | 1023 |
| | HCDR3 | VGRFSIFGVELDN | 1024 |
| | HFR1 | QVQLVQSGAEVKKPGASVKVSCKAS | 190 |
| | | GYTFT | |
| | HFR2 | WVRQAPGQGLEWMG | 160 |
| | HFR3 | RVTMTRDTSISTAYMELSSLRSDDT | 1025 |
| | | AVYYCAR | |
| | HFR4 | WGQGTLVTVSS | 60 |
| | LC | QSALTQPPSASGSPGQSVTISCTGT | 1026 |
| | | SSDVGGYNFVSWYQQHPGKAPKLMI | |
| | | YEVSKRPSGVPDRFSGSKSGNTASL | |
| | | TVSGLQAEDEADYYCSSYGGSNNLI | |
| | | FGGGTRLTVLGQPKAAPSVTLFPPS | |
| | | SEELQANKATLVCLISDFYPGAVTV | |
| | | AWKADSSPVKAGVETTTPSKQSNNK | |
| | | YAASSYLSLTPEQWKSH | |
| | LC | QSALTQPPSASGSPGQSVTISCTGT | 1027 |
| | variable | SSDVGGYNFVSWYQQHPGKAPKLMI | |
| | | YEVSKRPSGVPDRFSGSKSGNTASL | |
| | | TVSGLQAEDEADYYCSSYGGSNNLI | |
| | | FGGGTRLTVL | |
| | LCDR1 | TGTSSDVGGYNFVS | 1028 |
| | LCDR2 | EVSKRPS | 94 |
| | LCDR3 | SSYGGSNNLI | 1029 |
| | LFR1 | QSALTQPPSASGSPGQSVTISC | 96 |
| | LFR2 | WYQQHPGKAPKLMIY | 67 |
| | LFR3 | GVPDRFSGSKSGNTASLTVSGLQAE | 1006 |
| | | DEADYYC | |
| | LFR4 | FGGGTRLTVL | 721 |
| S564-275 | HC | QVQLQESGPGLVKPSETLSLTCTVS | 1030 |
| | | GGSISSYYWSWIRQPPGKGLEWIGY | |
| | | IYYSGSTKYNPSLKSRVTISVDTSK | |
| | | KQFSLKLSSVTAADTAVYYCARHIK | |
| | | IGVVGGLTFDFWGQGTLVTVSSGSA | |
| | | SAPTLFPLVSCENSPSDTSSV | |
| | HC | QVQLQESGPGLVKPSETLSLTCTVS | 1031 |
| | variable | GGSISSYYWSWIRQPPGKGLEWIGY | |
| | | IYYSGSTKYNPSLKSRVTISVDTSK | |
| | | KQFSLKLSSVTAADTAVYYCARHIK | |
| | | IGVVGGLTFDFWGQGTLVTVSS | |
| | HCDR1 | SYYWS | 56 |
| | HCDR2 | YIYYSGSTKYNPSLKS | 333 |
| | HCDR3 | HIKIGVVGGLTFDF | 1032 |
| | HFR1 | QVQLQESGPGLVKPSETLSLTCTVS | 24 |
| | | GGSIS | |
| | HFR2 | WIRQPPGKGLEWIG | 7 |
| | HFR3 | RVTISVDTSKKQFSLKLSSVTAADT | 1033 |
| | | AVYYCAR | |
| | HFR4 | WGQGTLVTVSS | 60 |
| | LC | DIQMTQSPSSLSASIGDRVTITCRA | 1034 |
| | | SQSISTYLNWYQQKPGKAPKLLIYA | |
| | | ASSLQSGVPSRFSGSGSGADFTLTI | |
| | | SSLQPEDFATYYCQQSYSTPLTFGG | |
| | | GTKVEIKRTVAAPSVFIFPPSDEQL | |
| | | KSGTASVVCLLNNFYPREAKVQWKV | |
| | | DNA | |
| | LC | DIQMTQSPSSLSASIGDRVTITCRA | 1035 |
| | variable | SQSISTYLNWYQQKPGKAPKLLIYA | |
| | | ASSLQSGVPSRFSGSGSGADFTLTI | |
| | | SSLQPEDFATYYCQQSYSTPLTFGG | |
| | | GTKVEIK | |
| | LCDR1 | RASQSISTYLN | 1036 |
| | LCDR2 | AASSLQS | 249 |
| | LCDR3 | QQSYSTPLT | 1037 |
| | LFR1 | DIQMTQSPSSLSASIGDRVTITC | 1038 |
| | LFR2 | WYQQKPGKAPKLLIY | 252 |
| | LFR3 | GVPSRFSGSGSGADFTLTISSLQPE | 1039 |
| | | DFATYYC | |
| | LFR4 | FGGGTKVEIK | 85 |
| S564-287 | HC | QVQLVQSGAEVKKPGASVKVSCKAS | 1040 |
| | | GYTFTGYYMHWVRQAPGQGLEWMGW | |
| | | INPNSGGTNYAQKFQGRVTMTRDTS | |
| | | ISTAYMELSRLRCDDTAVYYCARAS | |
| | | TPYSSGSWADYWGQGTLVTVSSGSA | |
| | | SAPTLFPLVSCENSPSDTSSV | |
| | HC | QVQLVQSGAEVKKPGASVKVSCKAS | 1041 |
| | variable | GYTFTGYYMHWVRQAPGQGLEWMGW | |
| | | INPNSGGTNYAQKFQGRVTMTRDTS | |
| | | ISTAYMELSRLRCDDTAVYYCARAS | |
| | | TPYSSGSWADYWGQGTLVTVSS | |
| | HCDR1 | GYYMH | 187 |
| | HCDR2 | WINPNSGGTNYAQKFQG | 953 |
| | HCDR3 | ASTPYSSGSWADY | 1042 |
| | HFR1 | QVQLVQSGAEVKKPGASVKVSCKAS | 190 |
| | | GYTFT | |
| | HFR2 | WVRQAPGQGLEWMG | 160 |
| | HFR3 | RVTMTRDTSISTAYMELSRLRCDDT | 1043 |
| | | AVYYCAR | |
| | HFR4 | WGQGTLVTVSS | 60 |
| | LC | QSALTQPASVSGSPGQSITISCTGT | 1044 |
| | | SSDVGGYNYVSWYQQHPGKAPKLMI | |
| | | YDVSNRPSGVSNRFSGSKSGNTASL | |
| | | TISGLQAEDEADYYCSSYASSSTWV | |
| | | FGGGTKLTVLGQPKAAPSVTLFPPS | |
| | | SEELQANKATLVCLISDFYPGAVTV | |
| | | AWKADSSPVKAGVETTTPSKQSNNK | |
| | | YAASSYLSLT | |
| | LC | QSALTQPASVSGSPGQSITISCTGT | 1045 |
| | variable | SSDVGGYNYVSWYQQHPGKAPKLMI | |
| | | YDVSNRPSGVSNRFSGSKSGNTASL | |
| | | TISGLQAEDEADYYCSSYASSSTWV | |
| | | FGGGTKLTVL | |
| | LCDR1 | TGTSSDVGGYNYVS | 63 |
| | LCDR2 | DVSNRPS | 64 |
| | LCDR3 | SSYASSSTWV | 1046 |
| | LFR1 | QSALTQPASVSGSPGQSITISC | 66 |
| | LFR2 | WYQQHPGKAPKLMIY | 67 |
| | LFR3 | GVSNRFSGSKSGNTASLTISGLQAE | 68 |
| | | DEADYYC | |
| | LFR4 | FGGGTKLTVL | 69 |
| S166-32 | HC | QVQLVESGGGLVKPGGSLRLSCAAS | 1047 |
| | | GFTFSDYYMSWIRQAPGKGLEWVSY | |
| | | ISISDTTIYYADAVQGRFTMSRDNA | |
| | | KNSLYLQMNSLKAEDTAVYYCARAS | |
| | | PYCGGDCSFGNAFDIWGLGTMVTVS | |
| | | S | |
| | HC | QVQLVESGGGLVKPGGSLRLSCAAS | 1048 |
| | variable | GFTFSDYYMSWIRQAPGKGLEWVSY | |
| | | ISISDTTIYYADAVQGRFTMSRDNA | |
| | | KNSLYLQMNSLKAEDTAVYYCAR | |
| | HCDR1 | DYYMS | 1049 |
| | HCDR2 | YISISDTTIYYADAVQG | 1050 |
| | HCDR3 | ASPYCGGDCSFGNAFDI | 1051 |
| | HFR1 | QVQLVESGGGLVKPGGSLRLSCAAS | 1052 |
| | | GFTFS | |
| | HFR2 | WIRQAPGKGLEWVS | 1053 |
| | HFR3 | RFTMSRDNAKNSLYLQMNSLKAEDT | 1054 |
| | | AVYYCAR | |
| | HFR4 | WGLGTMVTVSS | 1055 |
| | LC | DIQMTQSPSTLSASVGDRVTITCRA | 1056 |
| | | SQSIFSWLAWYQQKPGKAPKLLIYD | |
| | | ASSLESGVPSRFSGSGSGTEFTLTI | |
| | | SSLQPDDFATYYCQQYNSYWTFGQG | |
| | | TKVEIK | |
| | LC | DIQMTQSPSTLSASVGDRVTITCRA | 1057 |
| | variable | SQSIFSWLAWYQQKPGKAPKLLIYD | |
| | | ASSLESGVPSRFSGSGSGTEFTLTI | |
| | | SSLQPDDFATYYCQQYNSY | |
| | LCDR1 | RASQSIFSWLA | 1058 |
| | LCDR2 | DASSLES | 387 |
| | LCDR3 | QQYNSYWT | 1059 |
| | LFR1 | DIQMTQSPSTLSASVGDRVTITC | 523 |
| | LFR2 | WYQQKPGKAPKLLIY | 252 |
| | LFR3 | GVPSRFSGSGSGTEFTLTISSLQPD | 524 |
| | | DFATYYC | |
| | LFR4 | FGQGTKVEIK | 53 |
| S305-223 | HC | QVQLVESGGGVVQPGRSLRLSCAAS | 1060 |
| | | GFTFRNFGMHWVRQAPGKGLEWVAF | |
| | | IWTAESDKFYADSVKGRFTVSRDNS | |
| | | KNTLYLEMNSLRAEDTAVYYCTKAM | |
| | | DVWGRGTTVTVSS | |
| | HC | QVQLVESGGGVVQPGRSLRLSCAAS | 1061 |
| | variable | GFTFRNFGMHWVRQAPGKGLEWVAF | |
| | | IWTAESDKFYADSVKGRFTVSRDNS | |
| | | KNTLYLEMNSLRAEDTAVYYCTK | |
| | HCDR1 | NFGMH | 1062 |
| | HCDR2 | FIWTAESDKFYADSVKG | 1063 |
| | HCDR3 | AMDV | 1064 |
| | HFR1 | QVQLVESGGGVVQPGRSLRLSCAAS | 1065 |
| | | GFTFR | |
| | HFR2 | WVRQAPGKGLEWVA | 145 |
| | HFR3 | RFTVSRDNSKNTLYLEMNSLRAEDT | 1066 |
| | | AVYYCTK | |
| | HFR4 | WGRGTTVTVSS | 1067 |
| | LC | EIVLTQSPATLSLSPGERATLSCRA | 1068 |
| | | SQSVSTSLAWYQQKCGQAPRLLIYD | |
| | | ASNRATGIPARFSGSGSGTDFTLTI | |
| | | SSLEPEDFAVYYCQQRGNWPFTFGP | |
| | | GTRVDIK | |
| | LC | EIVLTQSPATLSLSPGERATLSCRA | 1069 |
| | variable | SQSVSTSLAWYQQKCGQAPRLLIYD | |
| | | ASNRATGIPARFSGSGSGTDFTLTI | |
| | | SSLEPEDFAVYYCQQRGNWP | |
| | LCDR1 | RASQSVSTSLA | 1070 |
| | LCDR2 | DASNRAT | 441 |
| | LCDR3 | QQRGNWPFT | 1071 |
| | LFR1 | EIVLTQSPATLSLSPGERATLSC | 181 |
| | LFR2 | WYQQKCGQAPRLLIY | 1072 |
| | LFR3 | GIPARFSGSGSGTDFTLTISSLEPE | 183 |
| | | DFAVYYC | |
| | LFR4 | FGPGTRVDIK | 1073 |
| S305-399 | HC | QVQLVQSGAEVKKPGASVKVSCKVS | 1074 |
| | | GYTLTELSMHWVRQAPGKGLEWMGG | |
| | | FDPEDGETIYAQKFQGRVTMTEDTS | |
| | | TDTAYMELSSLRSEDTAVYYCATGG | |
| | | LGCSNGVCNNWFDPWGLGTLVTVSS | |
| | HC | QVQLVQSGAEVKKPGASVKVSCKVS | 1075 |
| | variable | GYTLTELSMHWVRQAPGKGLEWMGG | |
| | | FDPEDGETIYAQKFQGRVTMTEDTS | |
| | | TDTAYMELSSLRSEDTAVYYCAT | |
| | HCDR1 | ELSMH | 457 |
| | HCDR2 | GFDPEDGETIYAQKFQG | 458 |
| | HCDR3 | GGLGCSNGVCNNWFDP | 1076 |
| | HFR1 | QVQLVQSGAEVKKPGASVKVSCKVS | 1077 |
| | | GYTLT | |
| | HFR2 | WVRQAPGKGLEWMG | 42 |
| | HFR3 | RVTMTEDTSTDTAYMELSSLRSEDT | 1078 |
| | | AVYYCAT | |
| | HFR4 | WGLGTLVTVSS | 1079 |
| | LC | EIVMTQSPATLSVSPGERATLSCRA | 1080 |
| | | SQSITSNLAWYQQKPGQAPRLLIYG | |
| | | ASTRATGIPARFSGSGSGTEFTLTI | |
| | | SNLQSEDFAVYYCQQYNNWPLTFGQ | |
| | | GTKVEIK | |
| | LC | EIVMTQSPATLSVSPGERATLSCRA | 1081 |
| | variable | SQSITSNLAWYQQKPGQAPRLLIYG | |
| | | ASTRATGIPARFSGSGSGTEFTLTI | |
| | | SNLQSEDFAVYYCQQYNNWP | |
| | LCDR1 | RASQSITSNLA | 1082 |
| | LCDR2 | GASTRAT | 208 |
| | LCDR3 | QQYNNWPLT | 1083 |
| | LFR1 | EIVMTQSPATLSVSPGERATLSC | 210 |
| | LFR2 | WYQQKPGQAPRLLIY | 182 |
| | LFR3 | GIPARFSGSGSGTEFTLTISNLQSE | 1084 |
| | | DFAVYYC | |
| | LFR4 | FGQGTKVEIK | 53 |
| S305-1456 | HC | QVQLVQSGAEVKKPGASVKVSCKVS | 1085 |
| | | GYTLTELSMHWVRQAPGKGLEWMGG | |
| | | FDPEDAETIYAQKFQGRVTMTEDTS | |
| | | TDTAYMELSSLRSEDTAVYYCATGG | |
| | | FPVNSLYDILTGYLDYWGQGTLVTV | |
| | | SS | |
| | HC | QVQLVQSGAEVKKPGASVKVSCKVS | 1086 |
| | variable | GYTLTELSMHWVRQAPGKGLEWMGG | |
| | | FDPEDAETIYAQKFQGRVTMTEDTS | |
| | | TDTAYMELSSLRSEDTAVYYCAT | |
| | HCDR1 | ELSMH | 457 |
| | HCDR2 | GFDPEDAETIYAQKFQG | 1087 |
| | HCDR3 | GGFPVNSLYDILTGYLDY | 1088 |
| | HFR1 | QVQLVQSGAEVKKPGASVKVSCKVS | 1077 |
| | | GYTLT | |
| | HFR2 | WVRQAPGKGLEWMG | 42 |
| | HFR3 | RVTMTEDTSTDTAYMELSSLRSEDT | 1078 |
| | | AVYYCAT | |
| | HFR4 | WGQGTLVTVSS | 60 |
| | LC | EIVMTQSPATLSVSPGERATLSCRA | 1089 |
| | | SQNVSSNLAWYQQKPGQAPRLLIYG | |
| | | ASTRATGIPARFSGSGSGTEFTLTI | |
| | | SSLQSEDFAVYYCQQYNNWPHTFGP | |
| | | GTKVDIK | |
| | LC | EIVMTQSPATLSVSPGERATLSCRA | 1090 |
| | variable | SQNVSSNLAWYQQKPGQAPRLLIYG | |
| | | ASTRATGIPARFSGSGSGTEFTLTI | |
| | | SSLQSEDFAVYYCQQYNNWP | |
| | LCDR1 | RASQNVSSNLA | 1091 |
| | LCDR2 | GASTRAT | 208 |
| | LCDR3 | QQYNNWPHT | 1092 |
| | LFR1 | EIVMTQSPATLSVSPGERATLSC | 210 |
| | LFR2 | WYQQKPGQAPRLLIY | 182 |
| | LFR3 | GIPARFSGSGSGTEFTLTISSLQSE | 211 |
| | | DFAVYYC | |
| | LFR4 | FGPGTKVDIK | 443 |
| R125-306 | HC | QVQMVESGGGVVQPGGSLRLSCAVS | 1093 |
| | | GFTFNNFGMHWVRQAPGKGLEWVAF | |
| | | ISYEGSKKSYADSVKGRFTISRDSS | |
| | | KNTLYLQMNSLRPEDTSVYYCAKEL | |
| | | AIFMIYAGRYGLDVWGQGTTVTVSS | |
| | HC | QVQMVESGGGVVQPGGSLRLSCAVS | 1094 |
| | variable | GFTFNNFGMHWVRQAPGKGLEWVAF | |
| | | ISYEGSKKSYADSVKGRFTISRDSS | |
| | | KNTLYLQMNSLRPEDTSVYYCAK | |
| | HCDR1 | NFGMH | 1062 |
| | HCDR2 | FISYEGSKKSYADSVKG | 1095 |
| | HCDR3 | ELAIFMIYAGRYGLDV | 1096 |
| | HFR1 | QVQMVESGGGVVQPGGSLRLSCAVS | 1097 |
| | | GFTFN | |
| | HFR2 | WVRQAPGKGLEWVA | 145 |
| | HFR3 | RFTISRDSSKNTLYLQMNSLRPEDT | 1098 |
| | | SVYYCAK | |
| | HFR4 | WGQGTTVTVSS | 147 |
| | LC | SALTQPASVSGSPGQSITISCTGIY | 1099 |
| | | SDVDDYTSVSWYQQHPGKAPTLIIY | |
| | | DVTKRPSGVSNRFSASNSDNTASLT | |
| | | ISGLQAEDEAEYYCCSRGSATNSYV | |
| | | FGTGTKVTVL | |
| | LC | SALTQPASVSGSPGQSITISCTGIY | 1100 |
| | variable | SDVDDYTSVSWYQQHPGKAPTLIIY | |
| | | DVTKRPSGVSNRFSASNSDNTASLT | |
| | | ISGLQAEDEAEYYCCSRGS | |
| | LCDR1 | TGIYSDVDDYTSVS | 1101 |
| | LCDR2 | DVTKRPS | 1102 |
| | LCDR3 | CSRGSATNSYV | 1103 |
| | LFR1 | SALTQPASVSGSPGQSITISC | 1104 |
| | LFR2 | WYQQHPGKAPTLIIY | 1105 |
| | LFR3 | GVSNRFSASNSDNTASLTISGLQAE | 1106 |
| | | DEAEYYC | |
| | LFR4 | FGTGTKVTVL | 18 |
| R125-444 | HC | QVQLQESGPGLVKPSETLSLTCNVS | 1107 |
| | | GGSVKFFYWSWIRQPPGKGLEWIGY | |
| | | IYYSGSTNYNPSLKSRVTMSVDSPN | |
| | | NQFSLKLRSVTAADTAVYYCARVGR | |
| | | DCSSGICRTYDYYAMDVWGQGTTVT | |
| | | VSS | |
| | HC | QVQLQESGPGLVKPSETLSLTCNVS | 1108 |
| | variable | GGSVKFFYWSWIRQPPGKGLEWIGY | |
| | | IYYSGSTNYNPSLKSRVTMSVDSPN | |
| | | NQFSLKLRSVTAADTAVYYCAR | |
| | HCDR1 | FFYWS | 1109 |
| | HCDR2 | YIYYSGSTNYNPSLKS | 4 |
| | HCDR3 | VGRDCSSGICRTYDYYAMDV | 1110 |
| | HFR1 | QVQLQESGPGLVKPSETLSLTCNVS | 1111 |
| | | GGSVK | |
| | HFR2 | WIRQPPGKGLEWIG | 7 |
| | HFR3 | RVTMSVDSPNNQFSLKLRSVTAADT | 1112 |
| | | AVYYCAR | |
| | HFR4 | WGQGTTVTVSS | 147 |
| | LC | QSVLTQPPSLSGAPGQRVTISCTGS | 1113 |
| | | RSNIGAGYAVHWYQQLPGTAPKLLI | |
| | | SENTNGPSGVPDRFSGSKSDSSASL | |
| | | AITDLQAADEADYYCQSYDGSLSGW | |
| | | VFGGGTKLTVL | |
| | LC | QSVLTQPPSLSGAPGQRVTISCTGS | 1114 |
| | variable | RSNIGAGYAVHWYQQLPGTAPKLLI | |
| | | SENTNGPSGVPDRFSGSKSDSSASL | |
| | | AITDLQAADEADYYCQSYDGSLSG | |
| | LCDR1 | TGSRSNIGAGYAVH | 1115 |
| | LCDR2 | ENTNGPS | 1116 |
| | LCDR3 | QSYDGSLSGWV | 1117 |
| | LFR1 | QSVLTQPPSLSGAPGQRVTISC | 1118 |
| | LFR2 | WYQQLPGTAPKLLIS | 1119 |
| | LFR3 | GVPDRFSGSKSDSSASLAITDLQAA | 1120 |
| | | DEADYYC | |
| | LFR4 | FGGGTKLTVL | 69 |
| R3-428 | HC | QVTLKESGPVLVKPTETLTLTCTVS | 1121 |
| | | GFSPSNARMGVSWIRQPPGKALEWL | |
| | | AHVYSNDEKSYSTSLKRRLTISKDT | |
| | | SKRQVVLIMTNLDPADTGTYYCARA | |
| | | QDPRIRFGELLPVYFDNWGQGTLVT | |
| | | VSS | |
| | HC | QVTLKESGPVLVKPTETLTLTCTVS | 1122 |
| | variable | GFSPSNARMGVSWIRQPPGKALEWL | |
| | | AHVYSNDEKSYSTSLKRRLTISKDT | |
| | | SKRQVVLIMTNLDPADTGTYYCAR | |
| | HCDR1 | NARMGVS | 1123 |
| | HCDR2 | HVYSNDEKSYSTSLKR | 1124 |
| | HCDR3 | AQDPRIRFGELLPVYFDN | 1125 |
| | HFR1 | QVTLKESGPVLVKPTETLTLTCTVS | 1126 |
| | | GFSPS | |
| | HFR2 | WIRQPPGKALEWLA | 476 |
| | HFR3 | RLTISKDTSKRQVVLIMTNLDPADT | 1127 |
| | | GTYYCAR | |
| | HFR4 | WGQGTLVTVSS | 60 |
| | LC | DIQMTQSPSSLSAFVGDRVTISCRA | 1128 |
| | | SQSIVSYLNWYQQKPGKAPKLLLYS | |
| | | ASTLQSGVPSRFSGSGSGTDFTLTI | |
| | | SSLQPEDFATYYCQQGYTTPWTFGQ | |
| | | GTKVEIK | |
| | LC | DIQMTQSPSSLSAFVGDRVTISCRA | 1129 |
| | variable | SQSIVSYLNWYQQKPGKAPKLLLYS | |
| | | ASTLQSGVPSRFSGSGSGTDFTLTI | |
| | | SSLQPEDFATYYCQQGYTTP | |
| | LCDR1 | RASQSIVSYLN | 1130 |
| | LCDR2 | SASTLQS | 1131 |
| | LCDR3 | QQGYTTPWT | 1132 |
| | LFR1 | DIQMTQSPSSLSAFVGDRVTISC | 1133 |
| | LFR2 | WYQQKPGKAPKLLLY | 1134 |
| | LFR3 | GVPSRFSGSGSGTDFTLTISSLQPE | 253 |
| | | DFATYYC | |
| | LFR4 | FGQGTKVEIK | 53 |
| R478910- | HC | EVQLLESGGGLVLPGGSLRLSCAAS | 1135 |
| 171 | | GFSFSSYAMSWVRQAPGKGLEWVSG | |
| | | ISGRGTSTYYADSVKGRFTISRDNS | |
| | | KNTLYLQMNSLRAEDTAVYYCAKDR | |
| | | VSYGSPYYFDYWGQGTLVTVSS | |
| | HC | EVQLLESGGGLVLPGGSLRLSCAAS | 1136 |
| | variable | GFSFSSYAMSWVRQAPGKGLEWVSG | |
| | | ISGRGTSTYYADSVKGRFTISRDNS | |
| | | KNTLYLQMNSLRAEDTAVYYCAK | |
| | HCDR1 | SYAMS | 610 |
| | HCDR2 | GISGRGTSTYYADSVKG | 1137 |
| | HCDR3 | DRVSYGSPYYFDY | 1138 |
| | HFR1 | EVQLLESGGGLVLPGGSLRLSCAAS | 1139 |
| | | GFSFS | |
| | HFR2 | WVRQAPGKGLEWVS | 130 |
| | HFR3 | RFTISRDNSKNTLYLQMNSLRAEDT | 499 |
| | | AVYYCAK | |
| | HFR4 | WGQGTLVTVSS | 60 |
| | LC | DIVLTQSPATLSLSPGERATLSCGA | 1140 |
| | | SQSVSSNYLAWYQQEPGLAPRLLIY | |
| | | DASSRATGIPDRFSGSGSGTDFTLT | |
| | | ISRLEPEDFAVYYCQQYGSSSTFGQ | |
| | | GTRLEIK | |
| | LC | DIVLTQSPATLSLSPGERATLSCGA | 1141 |
| | variable | SQSVSSNYLAWYQQEPGLAPRLLIY | |
| | | DASSRATGIPDRFSGSGSGTDFTLT | |
| | | ISRLEPEDFAVYYCQQYGSS | |
| | LCDR1 | GASQSVSSNYLA | 1142 |
| | LCDR2 | DASSRAT | 1143 |
| | LCDR3 | QQYGSSST | 1144 |
| | LFR1 | DIVLTQSPATLSLSPGERATLSC | 1145 |
| | LFR2 | WYQQEPGLAPRLLIY | 1146 |
| | LFR3 | GIPDRFSGSGSGTDFTLTISRLEPE | 196 |
| | | DFAVYYC | |
| | LFR4 | FGQGTRLEIK | 701 |
| R478910-23 | HC | QVQLVESGGGVVQPGRSLRLSCAAS | 1147 |
| | | GFTFSSYGMHWVRQAPGKGLEWVAV | |
| | | IWYDGSNKYYADSVKGRFTISRDNS | |
| | | KNTLYLQMNSLRAEDTAVYYCARAP | |
| | | DYWGQGTLVTVSS | |
| | HC | QVQLVESGGGVVQPGRSLRLSCAAS | 1148 |
| | variable | GFTFSSYGMHWVRQAPGKGLEWVAV | |
| | | IWYDGSNKYYADSVKGRFTISRDNS | |
| | | KNTLYLQMNSLRAEDTAVYYCAR | |
| | HCDR1 | SYGMH | 141 |
| | HCDR2 | VIWYDGSNKYYADSVKG | 142 |
| | HCDR3 | APDY | 1149 |
| | HFR1 | QVQLVESGGGVVQPGRSLRLSCAAS | 144 |
| | | GFTFS | |
| | HFR2 | WVRQAPGKGLEWVA | 145 |
| | HFR3 | RFTISRDNSKNTLYLQMNSLRAEDT | 146 |
| | | AVYYCAR | |
| | HFR4 | WGQGTLVTVSS | 60 |
| | LC | AIQMTQSPSSLSASVGDRVTITCRA | 1150 |
| | | SQGIRNDLGWYQQKPGKAPKLLIYA | |
| | | ASSLQSGVPSRFSGSGSGTDFTLTI | |
| | | SSLQPEDFATYYCLQDYNYPYTFGQ | |
| | | GTKLEIK | |
| | LC | AIQMTQSPSSLSASVGDRVTITCRA | 1151 |
| | variable | SQGIRNDLGWYQQKPGKAPKLLIYA | |
| | | ASSLQSGVPSRFSGSGSGTDFTLTI | |
| | | SSLQPEDFATYYCLQDYNYP | |
| | LCDR1 | RASQGIRNDLG | 1152 |
| | LCDR2 | AASSLQS | 249 |
| | LCDR3 | LQDYNYPYT | 1153 |
| | LFR1 | AIQMTQSPSSLSASVGDRVTITC | 1154 |
| | LFR2 | WYQQKPGKAPKLLIY | 252 |
| | LFR3 | GVPSRFSGSGSGTDFTLTISSLQPE | 253 |
| | | DFATYYC | |
| | LFR4 | FGQGTKLEIK | 380 |
| R478910-25 | HC | EVQLLESGGGLVQPGGSLRLSCAVS | 1155 |
| | | GFTVSNYGLSWVRQGPGKGLEWVAA | |
| | | ISGSGGRTYYADSVKGRFTISRDNS | |
| | | KNTLFLQLNSLRAEDTAVYYCAKGR | |
| | | DELVVGATQDYWGQGTLVTVSS | |
| | HC | EVQLLESGGGLVQPGGSLRLSCAVS | 1156 |
| | variable | GFTVSNYGLSWVRQGPGKGLEWVAA | |
| | | ISGSGGRTYYADSVKGRFTISRDNS | |
| | | KNTLFLQLNSLRAEDTAVYYCAK | |
| | HCDR1 | NYGLS | 1157 |
| | HCDR2 | AISGSGGRTYYADSVKG | 1158 |
| | HCDR3 | GRDELVVGATQDY | 1159 |
| | HFR1 | EVQLLESGGGLVQPGGSLRLSCAVS | 1160 |
| | | GFTVS | |
| | HFR2 | WVRQGPGKGLEWVA | 1161 |
| | HFR3 | RFTISRDNSKNTLFLQLNSLRAEDT | 1162 |
| | | AVYYCAK | |
| | HFR4 | WGQGTLVTVSS | 60 |
| | LC | DIQMTQSPSSLSASVGDRVTITCRA | 1163 |
| | | NQNIRSYLNWYQQTPGKAPKLLIYA | |
| | | TSSLQSGVPSRFSGSGSGTQFTLTI | |
| | | SSLQPEDFATYYCQQSYSIPFDFGP | |
| | | GTKVDIK | |
| | LC | DIQMTQSPSSLSASVGDRVTITCRA | 1164 |
| | variable | NQNIRSYLNWYQQTPGKAPKLLIYA | |
| | | TSSLQSGVPSRFSGSGSGTQFTLTI | |
| | | SSLQPEDFATYYCQQSYSIP | |
| | LCDR1 | RANQNIRSYLN | 1165 |
| | LCDR2 | ATSSLQS | 1166 |
| | LCDR3 | QQSYSIPFD | 1167 |
| | LFR1 | DIQMTQSPSSLSASVGDRVTITC | 251 |
| | LFR2 | WYQQTPGKAPKLLIY | 453 |
| | LFR3 | GVPSRFSGSGSGTQFTLTISSLQPE | 1168 |
| | | DFATYYC | |
| | LFR4 | FGPGTKVDIK | 443 |
| R478910-3 | HC | EVQLLESGGDLVQPGGSLRLSCVAS | 1169 |
| | | GFTFRSYAMTWVRQAPGKGLEWVSS | |
| | | ISGSGGGTYYADSVKGRFTISRDNS | |
| | | KNTLFLQMNSLRAEDTAVYYCARGR | |
| | | EDWLLSLTYGYWGQGALVTVSS | |
| | HC | EVQLLESGGDLVQPGGSLRLSCVAS | 1170 |
| | variable | GFTFRSYAMTWVRQAPGKGLEWVSS | |
| | | ISGSGGGTYYADSVKGRFTISRDNS | |
| | | KNTLFLQMNSLRAEDTAVYYCAR | |
| | HCDR1 | SYAMT | 1171 |
| | HCDR2 | SISGSGGGTYYADSVKG | 1172 |
| | HCDR3 | GREDWLLSLTYGY | 1173 |
| | HFR1 | EVQLLESGGDLVQPGGSLRLSCVAS | 1174 |
| | | GFTFR | |
| | HFR2 | WVRQAPGKGLEWVS | 130 |
| | HFR3 | RFTISRDNSKNTLFLQMNSLRAEDT | 1175 |
| | | AVYYCAR | |
| | HFR4 | WGQGALVTVSS | 1176 |
| | LC | DIQMTQSPSSLSASVGDRVPITCRA | 1177 |
| | | SQSISSYLNWYQQRPGKAPKLLIYA | |
| | | ASSLQSGVPSRFSGSGSGTDFTLTI | |
| | | SSLQPEDFATYYCQQSYTIPPTFGG | |
| | | GTKVEIK | |
| | LC | DIQMTQSPSSLSASVGDRVPITCRA | 1178 |
| | variable | SQSISSYLNWYQQRPGKAPKLLIYA | |
| | | ASSLQSGVPSRFSGSGSGTDFTLTI | |
| | | SSLQPEDFATYYCQQSYTIPP | |
| | LCDR1 | RASQSISSYLN | 248 |
| | LCDR2 | AASSLQS | 249 |
| | LCDR3 | QQSYTIPPT | 1179 |
| | LFR1 | DIQMTQSPSSLSASVGDRVPITC | 1180 |
| | LFR2 | WYQQRPGKAPKLLIY | 1181 |
| | LFR3 | GVPSRFSGSGSGTDFTLTISSLQPE | 253 |
| | | DFATYYC | |
| | LFR4 | FGGGTKVEIK | 85 |
| R478910- | HC | EVQLVESGGGLVQPGGSLRLSCAAS | 1182 |
| 421 | | GFTFGSYWMNWVRQAPGKGLEWVAN | |
| | | INEDGSEKYYVDSVKGRFTISRDNA | |
| | | KNSLYLQMNSLRAEDTAVYYCARGH | |
| | | SLGEWGQGSPVTVSS | |
| | HC | EVQLVESGGGLVQPGGSLRLSCAAS | 1183 |
| | variable | GFTFGSYWMNWVRQAPGKGLEWVAN | |
| | | INEDGSEKYYVDSVKGRFTISRDNA | |
| | | KNSLYLQMNSLRAEDTAVYYCAR | |
| | HCDR1 | SYWMN | 1184 |
| | HCDR2 | NINEDGSEKYYVDSVKG | 1185 |
| | HCDR3 | GHSLGE | 1186 |
| | HFR1 | EVQLVESGGGLVQPGGSLRLSCAAS | 1187 |
| | | GFTFG | |
| | HFR2 | WVRQAPGKGLEWVA | 145 |
| | HFR3 | RFTISRDNAKNSLYLQMNSLRAEDT | 273 |
| | | AVYYCAR | |
| | HFR4 | WGQGSPVTVSS | 1188 |
| | LC | SSELTQDPAVSVALGQTVRITCQGD | 1189 |
| | | SLRSYSASWYQQKPGQAPVLVIYIK | |
| | | NKRPSGIPDRFSGSSSGNTASLTIT | |
| | | GAQAEDEADYYCNSRDSSGDHLVFG | |
| | | GGTKLTVL | |
| | LC | SSELTQDPAVSVALGQTVRITCQGD | 1190 |
| | variable | SLRSYSASWYQQKPGQAPVLVIYIK | |
| | | NKRPSGIPDRFSGSSSGNTASLTIT | |
| | | GAQAEDEADYYCNSRDSSGDHL | |
| | LCDR1 | QGDSLRSYSAS | 1191 |
| | LCDR2 | IKNKRPS | 1192 |
| | LCDR3 | NSRDSSGDHLV | 1193 |
| | LFR1 | SSELTQDPAVSVALGQTVRITC | 1194 |
| | LFR2 | WYQQKPGQAPVLVIY | 1195 |
| | LFR3 | GIPDRFSGSSSGNTASLTITGAQAE | 1196 |
| | | DEADYYC | |
| | LFR4 | FGGGTKLTVL | 69 |
| R478910-8 | HC | EVQLVESGGGLVQPGGSLRLSCAAS | 1197 |
| | | GFTFSSYSMNWVRQAPGKGLEWVSY | |
| | | ISSSSSTIYYADSVKGRFTISRDNA | |
| | | KNSLYLQMNSLRAEDTAVYYCARAN | |
| | | WNDMYFDLWGRGTLVTVSS | |
| | HC | EVQLVESGGGLVQPGGSLRLSCAAS | 1198 |
| | variable | GFTFSSYSMNWVRQAPGKGLEWVSY | |
| | | ISSSSSTIYYADSVKGRFTISRDNA | |
| | | KNSLYLQMNSLRAEDTAVYYCA | |
| | HCDR1 | SYSMN | 126 |
| | HCDR2 | YISSSSSTIYYADSVKG | 127 |
| | HCDR3 | ANWNDMYFDL | 1199 |
| | HFR1 | EVQLVESGGGLVQPGGSLRLSCAAS | 613 |
| | | GFTFS | |
| | HFR2 | WVRQAPGKGLEWVS | 130 |
| | HFR3 | RFTISRDNAKNSLYLQMNSLRAEDT | 273 |
| | | AVYYCAR | |
| | HFR4 | WGRGTLVTVSS | 9 |
| | LC | EIVLTQSPGTLSLSPGERATLSCRA | 1200 |
| | | SQSVSSSYLAWYQQKPGQAPRLLIY | |
| | | GASSRATGIPDRFSGSGSGTDFTLT | |
| | | ISRLEPEDFAVYYCQQYGSSPFGQG | |
| | | TKLEIK | |
| | LC | EIVLTQSPGTLSLSPGERATLSCRA | 1201 |
| | variable | SQSVSSSYLAWYQQKPGQAPRLLIY | |
| | | GASSRATGIPDRFSGSGSGTDFTLT | |
| | | ISRLEPEDFAVYYCQQYGSSP | |
| | LCDR1 | RASQSVSSSYLA | 338 |
| | LCDR2 | GASSRAT | 135 |
| | LCDR3 | QQYGSSP | 1202 |
| | LFR1 | EIVLTQSPGTLSLSPGERATLSC | 50 |
| | LFR2 | WYQQKPGQAPRLLIY | 182 |
| | LFR3 | GIPDRFSGSGSGTDFTLTISRLEPE | 196 |
| | | DFAVYYC | |
| | LFR4 | FGQGTKLEIK | 380 |
| S195-637 | HC | QVQLVESGGGVVQPGRSLRLSCAAS | 120 |
| | | GFTFSSNAMHWVRQAPGKGLEWVAV | |
| | | ISYDGDNKYYADSVKGRFTISRDNS | |
| | | KNTLYLQMNSLRTEDTAVYYCARSL | |
| | | GGNYFYGMDVWGQGTTVTVSS | |
| | HC | QVQLVESGGGVVQPGRSLRLSCAAS | 1204 |
| | variable | GFTFSSNAMHWVRQAPGKGLEWVAV | |
| | | ISYDGDNKYYADSVKGRFTISRDNS | |
| | | KNTLYLQMNSLRTEDTAVYYCA | |
| | HCDR1 | SNAMH | 1205 |
| | HCDR2 | VISYDGDNKYYADSVKG | 1206 |
| | HCDR3 | SLGGNYFYGMDV | 1207 |
| | HFR1 | QVQLVESGGGVVQPGRSLRLSCAAS | 144 |
| | | GFTFS | |
| | HFR2 | WVRQAPGKGLEWVA | 145 |
| | HFR3 | RFTISRDNSKNTLYLQMNSLRTEDT | 1208 |
| | | AVYYCAR | |
| | HFR4 | WGQGTTVTVSS | 147 |
| | LC | EIVLTQSPGTLSLSPGERATLSCRA | 1209 |
| | | SQSVSSSYLAWYQQKPGQAPRLLIY | |
| | | GASSRAAGIPDRFSGSGSGTDFTLT | |
| | | ISRLEPEDFAVYYCQQFGGSWTFGP | |
| | | GTKVEIK | |
| | LC | EIVLTQSPGTLSLSPGERATLSCRA | 1210 |
| | variable | SQSVSSSYLAWYQQKPGQAPRLLIY | |
| | | GASSRAAGIPDRFSGSGSGTDFTLT | |
| | | ISRLEPEDFAVYYCQQFGGS | |
| | LCDR1 | RASQSVSSSYLA | 338 |
| | LCDR2 | GASSRAA | 1211 |
| | LCDR3 | QQFGGSWT | 1212 |
| | LFR1 | EIVLTQSPGTLSLSPGERATLSC | 50 |
| | LFR2 | WYQQKPGQAPRLLIY | 182 |
| | LFR3 | GIPDRFSGSGSGTDFTLTISRLEPE | 196 |
| | | DFAVYYC | |
| | LFR4 | FGPGTKVEIK | 1213 |
| S380-1191 | HC | EVQLVESGGGLVQPGGSLRLSCAAS | 1214 |
| | | GFTFSGYIMNWVRQAPGKGLEWVSS | |
| | | ISGGSISISYAGSVKGRFTISRDNA | |
| | | KNSLYLQMNSLRAGDSAVYYCALTT | |
| | | FGVVTSYPSFDYWGQGTLVTVSS | |
| | HC | EVQLVESGGGLVQPGGSLRLSCAAS | 1215 |
| | variable | GFTFSGYIMNWVRQAPGKGLEWVSS | |
| | | ISGGSISISYAGSVKGRFTISRDNA | |
| | | KNSLYLQMNSLRAGDSAVYYCA | |
| | HCDR1 | GYIMN | 1216 |
| | HCDR2 | SISGGSISISYAGSVKG | 1217 |
| | HCDR3 | TTFGVVTSYPSFDY | 1218 |
| | HFR1 | EVQLVESGGGLVQPGGSLRLSCAAS | 613 |
| | | GFTFS | |
| | HFR2 | WVRQAPGKGLEWVS | 130 |
| | HFR3 | RFTISRDNAKNSLYLQMNSLRAGDS | 1219 |
| | | AVYYCAL | |
| | HFR4 | WGQGTLVTVSS | 60 |
| | LC | QSVLTQPPSVSGAPGQRVTISCTGS | 1220 |
| | | SSNIGAGYDVHWYQQLPGTAPKLLI | |
| | | YGNSNRPSGVPDRFSGSKSGTSASL | |
| | | AITGLQAEDEADYYCQSYDSSLSGY | |
| | | VFGGGTELTVL | |
| | LC | QSVLTQPPSVSGAPGQRVTISCTGS | 1221 |
| | variable | SSNIGAGYDVHWYQQLPGTAPKLLI | |
| | | YGNSNRPSGVPDRFSGSKSGTSASL | |
| | | AITGLQAEDEADYYCQSYDSSLSG | |
| | LCDR1 | TGSSSNIGAGYDVH | 673 |
| | LCDR2 | GNSNRPS | 350 |
| | LCDR3 | QSYDSSLSGYV | 1222 |
| | LFR1 | QSVLTQPPSVSGAPGQRVTISC | 537 |
| | LFR2 | WYQQLPGTAPKLLIY | 122 |
| | LFR3 | GVPDRFSGSKSGTSASLAITGLQAE | 539 |
| | | DEADYYC | |
| | LFR4 | FGGGTELTVL | 1223 |
| S451-101 | HC | QVQLVQSGAEVKKPGSSVKVSCKAS | 1224 |
| | | GGTFSNYAISWVRQAPGPGLEWMGG | |
| | | IIPFLGIANYAQKFQGRVTITADKS | |
| | | TSTAYMELSSLRSEDTAVYYCARAP | |
| | | GYSSVGSTNYFDPWGQGTLVTVSS | |
| | HC | QVQLVQSGAEVKKPGSSVKVSCKAS | 1225 |
| | variable | GGTFSNYAISWVRQAPGPGLEWMGG | |
| | | IIPFLGIANYAQKFQGRVTITADKS | |
| | | TSTAYMELSSLRSEDTAVYYCA | |
| | HCDR1 | NYAIS | 1226 |
| | HCDR2 | GIIPFLGIANYAQKFQG | 1227 |
| | HCDR3 | APGYSSVGSTNYFDP | 1228 |
| | HFR1 | QVQLVQSGAEVKKPGSSVKVSCKAS | 310 |
| | | GGTFS | |
| | HFR2 | WVRQAPGPGLEWMG | 1229 |
| | HFR3 | RVTITADKSTSTAYMELSSLRSEDT | 161 |
| | | AVYYCAR | |
| | HFR4 | WGQGTLVTVSS | 60 |
| | LC | QSVLTQPPSVSGAPGQRVTISCTGS | 1230 |
| | | SSNIGAGYDVHWYQQLPGAAPKLLI | |
| | | YANSNRPSGVPDRFSGSKSGTSASL | |
| | | AITGLQAEDEADYYCQSYDSSLSGY | |
| | | VFGTGTKVTVL | |
| | LC | QSVLTQPPSVSGAPGQRVTISCTGS | 1231 |
| | variable | SSNIGAGYDVHWYQQLPGAAPKLLI | |
| | | YANSNRPSGVPDRFSGSKSGTSASL | |
| | | AITGLQAEDEADYYCQSYDSSLSG | |
| | LCDR1 | TGSSSNIGAGYDVH | 673 |
| | LCDR2 | ANSNRPS | 1232 |
| | LCDR3 | QSYDSSLSGYV | 1222 |
| | LFR1 | QSVLTQPPSVSGAPGQRVTISC | 537 |
| | LFR2 | WYQQLPGAAPKLLIY | 719 |
| | LFR3 | GVPDRFSGSKSGTSASLAITGLQAE | 539 |
| | | DEADYYC | |
| | LFR4 | FGTGTKVTVL | 18 |
| S451-11 | HC | QVQLQESGPGLVEPSQTLSLTCTVS | 1233 |
| | | GGSISSGGYYWSWIRQHPGKGLEWI | |
| | | GYISYSGGSTYYNPSLKSVVTISLD | |
| | | TSKNQFSLKLSSVTAADTAVYYCAR | |
| | | VSYGSGSFRFDYWGQGTLVTVSS | |
| | HC | QVQLQESGPGLVEPSQTLSLTCTVS | 1234 |
| | variable | GGSISSGGYYWSWIRQHPGKGLEWI | |
| | | GYISYSGGSTYYNPSLKSVVTISLD | |
| | | TSKNQFSLKLSSVTAADTAVYYCAR | |
| | HCDR1 | SGGYYWS | 1235 |
| | HCDR2 | YISYSGGSTYYNPSLKS | 1236 |
| | HCDR3 | VSYGSGSFRFDY | 1237 |
| | HFR1 | QVQLQESGPGLVEPSQTLSLTCTVS | 1238 |
| | | GGSIS | |
| | HFR2 | WIRQHPGKGLEWIG | 1239 |
| | HFR3 | VVTISLDTSKNQFSLKLSSVTAADT | 1240 |
| | | AVYYCAR | |
| | HFR4 | WGQGTLVTVSS | 60 |
| | LC | QSALTQPRSVSGSPGQSVTISCTGT | 1241 |
| | | SSDVGGYNYFSWYQHHAGKAPKLMI | |
| | | YDVSKRPSGVPDRFSGSKSGNTASL | |
| | | TISGLQAEDEADYYCCSYAGTYTWV | |
| | | FGGGTKLTVL | |
| | LC | QSALTQPRSVSGSPGQSVTISCTGT | 1242 |
| | variable | SSDVGGYNYFSWYQHHAGKAPKLMI | |
| | | YDVSKRPSGVPDRFSGSKSGNTASL | |
| | | TISGLQAEDEADYYCCSYAGTYT | |
| | LCDR1 | TGTSSDVGGYNYFS | 1243 |
| | LCDR2 | DVSKRPS | 592 |
| | LCDR3 | CSYAGTYTWV | 1244 |
| | LFR1 | QSALTQPRSVSGSPGQSVTISC | 594 |
| | LFR2 | WYQHHAGKAPKLMIY | 1245 |
| | LFR3 | GVPDRFSGSKSGNTASLTISGLQAE | 1246 |
| | | DEADYYC | |
| | LFR4 | FGGGTKLTVL | 69 |
| S451-1101 | HC | QVQLQESGPGLVKPSETLSLTCTVS | 1247 |
| | | GGSINSYSWSWIRQPAGKGLEWIGR | |
| | | ISTSGSTNNNPSLKSRVTMSVDTSK | |
| | | DQFSLKLTSVTAADTAVYYCARING | |
| | | AAAGTPFDYWGQGTLVTVSS | |
| | HC | QVQLQESGPGLVKPSETLSLTCTVS | 1248 |
| | variable | GGSINSYSWSWIRQPAGKGLEWIGR | |
| | | ISTSGSTNNNPSLKSRVTMSVDTSK | |
| | | DQFSLKLTSVTAADTAVYYCAR | |
| | HCDR1 | SYSWS | 1249 |
| | HCDR2 | RISTSGSTNNNPSLKS | 1250 |
| | HCDR3 | INGAAAGTPFDY | 1251 |
| | HFR1 | QVQLQESGPGLVKPSETLSLTCTVS | 324 |
| | | GGSIN | |
| | HFR2 | WIRQPAGKGLEWIG | 25 |
| | HFR3 | RVTMSVDTSKDQFSLKLTSVTAADT | 1252 |
| | | AVYYCAR | |
| | HFR4 | WGQGTLVTVSS | 60 |
| | LC | DIQMTQSPSSLSASVGDRVTITCRA | 1253 |
| | | SQSISNYLNWYQQKPGKAPKLLIYA | |
| | | ASSLQSGVPSRFSGSGSGTDFTLTI | |
| | | SSLQPEDFATYYCQQSYRTFGQGTK | |
| | | VEIK | |
| | LC | DIQMTQSPSSLSASVGDRVTITCRA | 1254 |
| | variable | SQSISNYLNWYQQKPGKAPKLLIYA | |
| | | ASSLQSGVPSRFSGSGSGTDFTLTI | |
| | | SSLQPEDFATYYCQQSYRT | |
| | LCDR1 | RASQSISNYLN | 570 |
| | LCDR2 | AASSLQS | 249 |
| | LCDR3 | QQSYRT | 1255 |
| | LFR1 | DIQMTQSPSSLSASVGDRVTITC | 251 |
| | LFR2 | WYQQKPGKAPKLLIY | 252 |
| | LFR3 | GVPSRFSGSGSGTDFTLTISSLQPE | 253 |
| | | DFATYYC | |
| | LFR4 | FGQGTKVEIK | 53 |
| S451-1439 | HC | EVQLVESGGGLVQPGRSLRLSCAAS | 1256 |
| | | GFTFDDFAMHWVRQAPGKGLEWVSG | |
| | | ISWNGGIIGYADSVKARFTISRDNA | |
| | | KNSLYLQMNSLRAEDTALYYCAKTR | |
| | | GDYDYVWGSRSSNYYFDYWGQGTLV | |
| | | TVSS | |
| | HC | EVQLVESGGGLVQPGRSLRLSCAAS | 1257 |
| | variable | GFTFDDFAMHWVRQAPGKGLEWVSG | |
| | | ISWNGGIIGYADSVKARFTISRDNA | |
| | | KNSLYLQMNSLRAEDTALYYCA | |
| | HCDR1 | DFAMH | 1258 |
| | HCDR2 | GISWNGGIIGYADSVKA | 1259 |
| | HCDR3 | TRGDYDYVWGSRSSNYYFDY | 1260 |
| | HFR1 | EVQLVESGGGLVQPGRSLRLSCAAS | 1261 |
| | | GFTFD | |
| | HFR2 | WVRQAPGKGLEWVS | 130 |
| | HFR3 | RFTISRDNAKNSLYLQMNSLRAEDT | 1262 |
| | | ALYYCAK | |
| | HFR4 | WGQGTLVTVSS | 60 |
| | LC | DIQMTQSPSSLSASVGDRVTITCQA | 1263 |
| | | SQDISNYLNWYQKKPGKAPKLLIYD | |
| | | ATNLETGVPSRFSGSGSGTEFTFTI | |
| | | SSLQPEDIATYYCQQYDNVPPITFG | |
| | | PGTKVDMK | |
| | LC | DIQMTQSPSSLSASVGDRVTITCQA | 1264 |
| | variable | SQDISNYLNWYQKKPGKAPKLLIYD | |
| | | ATNLETGVPSRFSGSGSGTEFTFTI | |
| | | SSLQPEDIATYYCQQYDNVPP | |
| | LCDR1 | QASQDISNYLN | 769 |
| | LCDR2 | DATNLET | 1265 |
| | LCDR3 | QQYDNVPPIT | 1266 |
| | LFR1 | DIQMTQSPSSLSASVGDRVTITC | 251 |
| | LFR2 | WYQKKPGKAPKLLIY | 1267 |
| | LFR3 | GVPSRFSGSGSGTEFTFTISSLQPE | 1268 |
| | | DIATYYC | |
| | LFR4 | FGPGTKVDMK | 1269 |
| S451-1451 | HC | QVQLVESGGGVVQPGRSLRLSCAAS | 1203 |
| | | GFTFSSNAMHWVRQAPGKGLEWVAV | |
| | | ISYDGDNKYYADSVKGRFTISRDNS | |
| | | KNTLYLQMNSLRTEDTAVYYCARSL | |
| | | GGNYFYGMDVWGQGTTVTVSS | |
| | HC | QVQLVESGGGVVQPGRSLRLSCAAS | 1204 |
| | variable | GFTFSSNAMHWVRQAPGKGLEWVAV | |
| | | ISYDGDNKYYADSVKGRFTISRDNS | |
| | | KNTLYLQMNSLRTEDTAVYYCA | |
| | HCDR1 | SNAMH | 1205 |
| | HCDR2 | VISYDGDNKYYADSVKG | 1206 |
| | HCDR3 | SLGGNYFYGMDV | 1207 |
| | HFR1 | QVQLVESGGGVVQPGRSLRLSCAAS | 144 |
| | | GFTFS | |
| | HFR2 | WVRQAPGKGLEWVA | 145 |
| | HFR3 | RFTISRDNSKNTLYLQMNSLRTEDT | 1208 |
| | | AVYYCAR | |
| | HFR4 | WGQGTTVTVSS | 147 |
| | LC | EIVLTQSPGTLSLSPGERATLSCRA | 1209 |
| | | SQSVSSSYLAWYQQKPGQAPRLLIY | |
| | | GASSRAAGIPDRFSGSGSGTDFTLT | |
| | | ISRLEPEDFAVYYCQQFGGSWTFGP | |
| | | GTKVEIK | |
| | LC | EIVLTQSPGTLSLSPGERATLSCRA | 1210 |
| | variable | SQSVSSSYLAWYQQKPGQAPRLLIY | |
| | | GASSRAAGIPDRFSGSGSGTDFTLT | |
| | | ISRLEPEDFAVYYCQQFGGS | |
| | LCDR1 | RASQSVSSSYLA | 338 |
| | LCDR2 | GASSRAA | 1211 |
| | LCDR3 | QQFGGSWT | 1212 |
| | LFR1 | EIVLTQSPGTLSLSPGERATLSC | 50 |
| | LFR2 | WYQQKPGQAPRLLIY | 182 |
| | LFR3 | GIPDRFSGSGSGTDFTLTISRLEPE | 196 |
| | | DFAVYYC | |
| | LFR4 | FGPGTKVEIK | 1213 |
| S451-1477 | HC | EPQLVESGGGLVQPGGSLRLSCAGS | 1270 |
| | | GFGFISYPMNWVRQAPGKGPEWISN | |
| | | IRTTAEGGTFYADSVKGRFTMSRDD | |
| | | GKTSIYLQMNSLRDEDTATYYCARD | |
| | | SSYGFDLWGQGTVVTVSS | |
| | HC | EPQLVESGGGLVQPGGSLRLSCAGS | 1271 |
| | variable | GFGFISYPMNWVRQAPGKGPEWISN | |
| | | IRTTAEGGTFYADSVKGRFTMSRDD | |
| | | GKTSIYLQMNSLRDEDTATYYCAR | |
| | HCDR1 | SYPMN | 1272 |
| | HCDR2 | NIRTTAEGGTFYADSVKG | 1273 |
| | HCDR3 | DSSYGFDL | 1274 |
| | HFR1 | EPQLVESGGGLVQPGGSLRLSCAGS | 1275 |
| | | GFGFI | |
| | HFR2 | WVRQAPGKGPEWIS | 1276 |
| | HFR3 | RFTMSRDDGKTSIYLQMNSLRDEDT | 1277 |
| | | ATYYCAR | |
| | HFR4 | WGQGTVVTVSS | 421 |
| | LC | QSALTQPRSVSGSPGQSVTISCTGT | 1278 |
| | | SSDVGGYNYVSWYQQRPGKAPELMI | |
| | | YHVSERPSGVPDRFSGSKSGNTASL | |
| | | TISRLQAEDEADYYCCSYAGSHFWV | |
| | | FGGGTKLTVL | |
| | LC | QSALTQPRSVSGSPGQSVTISCTGT | 1279 |
| | variable | SSDVGGYNYVSWYQQRPGKAPELMI | |
| | | YHVSERPSGVPDRFSGSKSGNTASL | |
| | | TISRLQAEDEADYYCCSYAGSH | |
| | LCDR1 | TGTSSDVGGYNYVS | 63 |
| | LCDR2 | HVSERPS | 1280 |
| | LCDR3 | CSYAGSHFWV | 1281 |
| | LFR1 | QSALTQPRSVSGSPGQSVTISC | 594 |
| | LFR2 | WYQQRPGKAPELMIY | 1282 |
| | LFR3 | GVPDRFSGSKSGNTASLTISRLQAE | 1283 |
| | | DEADYYC | |
| | LFR4 | FGGGTKLTVL | 69 |
| S451-1503 | HC | EVQLVESGGGLVQPGGSLRLSCAAS | 1182 |
| | | GFTFGSYWMNWVRQAPGKGLEWVAN | |
| | | INEDGSEKYYVDSVKGRFTISRDNA | |
| | | KNSLYLQMNSLRAEDTAVYYCARGH | |
| | | SLGEWGQGSPVTVSS | |
| | HC | EVQLVESGGGLVQPGGSLRLSCAAS | 1183 |
| | variable | GFTFGSYWMNWVRQAPGKGLEWVAN | |
| | | INEDGSEKYYVDSVKGRFTISRDNA | |
| | | KNSLYLQMNSLRAEDTAVYYCAR | |
| | HCDR1 | SYWMN | 1184 |
| | HCDR2 | NINEDGSEKYYVDSVKG | 1185 |
| | HCDR3 | GHSLGE | 1186 |
| | HFR1 | EVQLVESGGGLVQPGGSLRLSCAAS | 1187 |
| | | GFTFG | |
| | HFR2 | WVRQAPGKGLEWVA | 145 |
| | HFR3 | RFTISRDNAKNSLYLQMNSLRAEDT | 273 |
| | | AVYYCAR | |
| | HFR4 | WGQGSPVTVSS | 1188 |
| | LC | SSELTQDPAVSVALGQTVRITCQGD | 1189 |
| | | SLRSYSASWYQQKPGQAPVLVIYIK | |
| | | NKRPSGIPDRFSGSSSGNTASLTIT | |
| | | GAQAEDEADYYCNSRDSSGDHLVFG | |
| | | GGTKLTVL | |
| | LC | SSELTQDPAVSVALGQTVRITCQGD | 1190 |
| | variable | SLRSYSASWYQQKPGQAPVLVIYIK | |
| | | NKRPSGIPDRFSGSSSGNTASLTIT | |
| | | GAQAEDEADYYCNSRDSSGDHL | |
| | LCDR1 | QGDSLRSYSAS | 1191 |
| | LCDR2 | IKNKRPS | 1192 |
| | LCDR3 | NSRDSSGDHLV | 1193 |
| | LFR1 | SSELTQDPAVSVALGQTVRITC | 1194 |
| | LFR2 | WYQQKPGQAPVLVIY | 1195 |
| | LFR3 | GIPDRFSGSSSGNTASLTITGAQAE | 1196 |
| | | DEADYYC | |
| | LFR4 | FGGGTKLTVL | 69 |
| S451-1522 | HC | EVQLVESGGGLVQPGGSLRLSCAAS | 1214 |
| | | GFTFSGYIMNWVRQAPGKGLEWVSS | |
| | | ISGGSISISYAGSVKGRFTISRDNA | |
| | | KNSLYLQMNSLRAGDSAVYYCALTT | |
| | | FGVVTSYPSFDYWGQGTLVTVSS | |
| | HC | EVQLVESGGGLVQPGGSLRLSCAAS | 1215 |
| | variable | GFTFSGYIMNWVRQAPGKGLEWVSS | |
| | | ISGGSISISYAGSVKGRFTISRDNA | |
| | | KNSLYLQMNSLRAGDSAVYYCA | |
| | HCDR1 | GYIMN | 1216 |
| | HCDR2 | SISGGSISISYAGSVKG | 1217 |
| | HCDR3 | TTFGVVTSYPSFDY | 1218 |
| | HFR1 | EVQLVESGGGLVQPGGSLRLSCAAS | 613 |
| | | GFTFS | |
| | HFR2 | WVRQAPGKGLEWVS | 130 |
| | HFR3 | RFTISRDNAKNSLYLQMNSLRAGDS | 1219 |
| | | AVYYCAL | |
| | HFR4 | WGQGTLVTVSS | 60 |
| | LC | QSVLTQPPSVSGAPGQRVTISCTGS | 1220 |
| | | SSNIGAGYDVHWYQQLPGTAPKLLI | |
| | | YGNSNRPSGVPDRFSGSKSGTSASL | |
| | | AITGLQAEDEADYYCQSYDSSLSGY | |
| | | VFGGGTELTVL | |
| | LC | QSVLTQPPSVSGAPGQRVTISCTGS | 1221 |
| | variable | SSNIGAGYDVHWYQQLPGTAPKLLI | |
| | | YGNSNRPSGVPDRFSGSKSGTSASL | |
| | | AITGLQAEDEADYYCQSYDSSLSG | |
| | LCDR1 | TGSSSNIGAGYDVH | 673 |
| | LCDR2 | GNSNRPS | 350 |
| | LCDR3 | QSYDSSLSGYV | 1222 |
| | LFR1 | QSVLTQPPSVSGAPGQRVTISC | 537 |
| | LFR2 | WYQQLPGTAPKLLIY | 122 |
| | LFR3 | GVPDRFSGSKSGTSASLAITGLQAE | 539 |
| | | DEADYYC | |
| | LFR4 | FGGGTELTVL | 1223 |
| S451-1921 | HC | QVQLQESGPGLVKPSQTLSLTCTVS | 1284 |
| | | GGSISSGDYYWSWIRQPPGKGLEWL | |
| | | GYIYYSGSTFYNPSLKSRVTISVDT | |
| | | SKNQFSLRLTSVTAADTAVYFCARE | |
| | | ENKFNYGHHPLNGVFAYWGQGTLVT | |
| | | VSS | |
| | HC | QVQLQESGPGLVKPSQTLSLTCTVS | 1285 |
| | variable | GGSISSGDYYWSWIRQPPGKGLEWL | |
| | | GYIYYSGSTFYNPSLKSRVTISVDT | |
| | | SKNQFSLRLTSVTAADTAVYFCAR | |
| | HCDR1 | SGDYYWS | 72 |
| | HCDR2 | YIYYSGSTFYNPSLKS | 1286 |
| | HCDR3 | EENKFNYGHHPLNGVFAY | 1287 |
| | HFR1 | QVQLQESGPGLVKPSQTLSLTCTVS | 1288 |
| | | GGSIS | |
| | HFR2 | WIRQPPGKGLEWLG | 1289 |
| | HFR3 | RVTISVDTSKNQFSLRLTSVTAADT | 1290 |
| | | AVYFCAR | |
| | HFR4 | WGQGTLVTVSS | 60 |
| | LC | DIVMTQSPDSLAVSLGERATINCKS | 1291 |
| | | SQSVLYSPNNKNYLAWYQQKPGQPP | |
| | | NLLIYWASTRESGVPDRFSGSGSGT | |
| | | DFTLTINSLQAEDVAVYYCQQSYNT | |
| | | PRTFGQGTKVEIK | |
| | LC | DIVMTQSPDSLAVSLGERATINCKS | 1292 |
| | variable | SQSVLYSPNNKNYLAWYQQKPGQPP | |
| | | NLLIYWASTRESGVPDRFSGSGSGT | |
| | | DFTLTINSLQAEDVAVYYCQQSYNT | |
| | | P | |
| | LCDR1 | KSSQSVLYSPNNKNYLA | 1293 |
| | LCDR2 | WASTRES | 30 |
| | LCDR3 | QQSYNTPRT | 1294 |
| | LFR1 | DIVMTQSPDSLAVSLGERATINC | 32 |
| | LFR2 | WYQQKPGQPPNLLIY | 1295 |
| | LFR3 | GVPDRFSGSGSGTDFTLTINSLQAE | 1296 |
| | | DVAVYYC | |
| | LFR4 | FGQGTKVEIK | 53 |
| S451-337 | HC | QVTLKESGPVLVKPTETLTLTCTVS | 1297 |
| | | GFSLINARLGVSWIRQPPGKALEWL | |
| | | AHIFSDDEKSYSTSLKSRLTISKDT | |
| | | SKSQVVLTMTNMDPVDTATYYCARI | |
| | | SWPPYGSGTYYIKAFDIWGQGTLVT | |
| | | VSS | |
| | HC | QVTLKESGPVLVKPTETLTLTCTVS | 1298 |
| | variable | GFSLINARLGVSWIRQPPGKALEWL | |
| | | AHIFSDDEKSYSTSLKSRLTISKDT | |
| | | SKSQVVLTMTNMDPVDTATYYCARI | |
| | HCDR1 | NARLGVS | 1299 |
| | HCDR2 | HIFSDDEKSYSTSLKS | 1300 |
| | HCDR3 | ISWPPYGSGTYYIKAFDI | 1301 |
| | HFR1 | QVTLKESGPVLVKPTETLTLTCTVS | 1302 |
| | | GFSLI | |
| | HFR2 | WIRQPPGKALEWLA | 476 |
| | HFR3 | RLTISKDTSKSQVVLTMTNMDPVDT | 1303 |
| | | ATYYCAR | |
| | HFR4 | WGQGTLVTVSS | 60 |
| | LC | QSALTQPVSVSGSPGQSITISCTGT | 1304 |
| | | SSDVGGYNYVSWYQQHPGKAPKLMI | |
| | | SEVSNRPSGVSNRFSGSKSGNTASL | |
| | | TISGLQAEDEADYYCSSYASSSTLW | |
| | | VFGGGTKLTVL | |
| | LC | QSALTQPVSVSGSPGQSITISCTGT | 1305 |
| | variable | SSDVGGYNYVSWYQQHPGKAPKLMI | |
| | | SEVSNRPSGVSNRFSGSKSGNTASL | |
| | | TISGLQAEDEADYYCSSYASSSTL | |
| | LCDR1 | TGTSSDVGGYNYVS | 63 |
| | LCDR2 | EVSNRPS | 151 |
| | LCDR3 | SSYASSSTLWV | 1306 |
| | LFR1 | QSALTQPVSVSGSPGQSITISC | 1307 |
| | LFR2 | WYQQHPGKAPKLMIS | 1308 |
| | LFR3 | GVSNRFSGSKSGNTASLTISGLQAE | 68 |
| | | DEADYYC | |
| | LFR4 | FGGGTKLTVL | 69 |
| S451-650 | HC | QVQLQESGPGLVKPSETLSLTCTVS | 1309 |
| | | GASISNFYWSWIRQPPGKGLEWIGY | |
| | | IYYSGSTNYNPSLKSRVTMSLDTSK | |
| | | NQFSLNLSSVTAADTAVYYCARIPN | |
| | | FWFGELLFDFWGHGTLVTVSS | |
| | HC | QVQLQESGPGLVKPSETLSLTCTVS | 1310 |
| | variable | GASISNFYWSWIRQPPGKGLEWIGY | |
| | | IYYSGSTNYNPSLKSRVTMSLDTSK | |
| | | NQFSLNLSSVTAADTAVYYCAR | |
| | HCDR1 | NFYWS | 1311 |
| | HCDR2 | YIYYSGSTNYNPSLKS | 4 |
| | HCDR3 | IPNFWFGELLFDF | 1312 |
| | HFR1 | QVQLQESGPGLVKPSETLSLTCTVS | 1313 |
| | | GASIS | |
| | HFR2 | WIRQPPGKGLEWIG | 7 |
| | HFR3 | RVTMSLDTSKNQFSLNLSSVTAADT | 1314 |
| | | AVYYCAR | |
| | HFR4 | WGHGTLVTVSS | 1315 |
| | LC | EIVLTQSPGTLSLSPGERATLSCRA | 1316 |
| | | SQSVSSNYLAWYQQKPGQAPRLLIY | |
| | | GASSRATGIPDRFSGSGSGTDFTLT | |
| | | ISRLEPEDFAVYYCQQYGSSPPITF | |
| | | GQGTRLEIK | |
| | LC | EIVLTQSPGTLSLSPGERATLSCRA | 1317 |
| | variable | SQSVSSNYLAWYQQKPGQAPRLLIY | |
| | | GASSRATGIPDRFSGSGSGTDFTLT | |
| | | ISRLEPEDFAVYYCQQYGSSPP | |
| | LCDR1 | RASQSVSSNYLA | 816 |
| | LCDR2 | GASSRAT | 135 |
| | LCDR3 | QQYGSSPPIT | 1318 |
| | LFR1 | EIVLTQSPGTLSLSPGERATLSC | 50 |
| | LFR2 | WYQQKPGQAPRLLIY | 182 |
| | LFR3 | GIPDRFSGSGSGTDFTLTISRLEPE | 196 |
| | | DFAVYYC | |
| | LFR4 | FGQGTRLEIK | 701 |
| S626-362 | HC | QVQLVQSGAEVKKPGSSVKVSCKAS | 1224 |
| | | GGTFSNYAISWVRQAPGPGLEWMGG | |
| | | IIPFLGIANYAQKFQGRVTITADKS | |
| | | TSTAYMELSSLRSEDTAVYYCARAP | |
| | | GYSSVGSTNYFDPWGQGTLVTVSS | |
| | HC | QVQLVQSGAEVKKPGSSVKVSCKAS | 1225 |
| | variable | GGTFSNYAISWVRQAPGPGLEWMGG | |
| | | IIPFLGIANYAQKFQGRVTITADKS | |
| | | TSTAYMELSSLRSEDTAVYYCA | |
| | HCDR1 | NYAIS | 1226 |
| | HCDR2 | GIIPFLGIANYAQKFQG | 1227 |
| | HCDR3 | APGYSSVGSTNYFDP | 1228 |
| | HFR1 | QVQLVQSGAEVKKPGSSVKVSCKAS | 310 |
| | | GGTFS | |
| | HFR2 | WVRQAPGPGLEWMG | 1229 |
| | HFR3 | RVTITADKSTSTAYMELSSLRSEDT | 161 |
| | | AVYYCAR | |
| | HFR4 | WGQGTLVTVSS | 60 |
| | LC | QSVLTQPPSVSGAPGQRVTISCTGS | 1230 |
| | | SSNIGAGYDVHWYQQLPGAAPKLLI | |
| | | YANSNRPSGVPDRFSGSKSGTSASL | |
| | | AITGLQAEDEADYYCQSYDSSLSGY | |
| | | VFGTGTKVTVL | |
| | LC | QSVLTQPPSVSGAPGQRVTISCTGS | 1231 |
| | variable | SSNIGAGYDVHWYQQLPGAAPKLLI | |
| | | YANSNRPSGVPDRFSGSKSGTSASL | |
| | | AITGLQAEDEADYYCQSYDSSLSG | |
| | LCDR1 | TGSSSNIGAGYDVH | 673 |
| | LCDR2 | ANSNRPS | 1232 |
| | LCDR3 | QSYDSSLSGYV | 1222 |
| | LFR1 | QSVLTQPPSVSGAPGQRVTISC | 537 |
| | LFR2 | WYQQLPGAAPKLLIY | 719 |
| | LFR3 | GVPDRFSGSKSGTSASLAITGLQAE | 539 |
| | | DEADYYC | |
| | LFR4 | FGTGTKVTVL | 18 |
| S626-651 | HC | EVQLVESGGGLVQPGRSLRLSCAAS | 1256 |
| | | GFTFDDFAMHWVRQAPGKGLEWVSG | |
| | | ISWNGGIIGYADSVKARFTISRDNA | |
| | | KNSLYLQMNSLRAEDTALYYCAKTR | |
| | | GDYDYVWGSRSSNYYFDYWGQGTLV | |
| | | TVSS | |
| | HC | EVQLVESGGGLVQPGRSLRLSCAAS | 1257 |
| | variable | GFTFDDFAMHWVRQAPGKGLEWVSG | |
| | | ISWNGGIIGYADSVKARFTISRDNA | |
| | | KNSLYLQMNSLRAEDTALYYCA | |
| | HCDR1 | DFAMH | 1258 |
| | HCDR2 | GISWNGGIIGYADSVKA | 1259 |
| | HCDR3 | TRGDYDYVWGSRSSNYYFDY | 1260 |
| | HFR1 | EVQLVESGGGLVQPGRSLRLSCAAS | 1261 |
| | | GFTFD | |
| | HFR2 | WVRQAPGKGLEWVS | 130 |
| | HFR3 | RFTISRDNAKNSLYLQMNSLRAEDT | 1262 |
| | | ALYYCAK | |
| | HFR4 | WGQGTLVTVSS | 60 |
| | LC | DIQMTQSPSSLSASVGDRVTITCQA | 1263 |
| | | SQDISNYLNWYQKKPGKAPKLLIYD | |
| | | ATNLETGVPSRFSGSGSGTEFTFTI | |
| | | SSLQPEDIATYYCQQYDNVPPITFG | |
| | | PGTKVDMK | |
| | LC | DIQMTQSPSSLSASVGDRVTITCQA | 1264 |
| | variable | SQDISNYLNWYQKKPGKAPKLLIYD | |
| | | ATNLETGVPSRFSGSGSGTEFTFTI | |
| | | SSLQPEDIATYYCQQYDNVPP | |
| | LCDR1 | QASQDISNYLN | 769 |
| | LCDR2 | DATNLET | 1265 |
| | LCDR3 | QQYDNVPPIT | 1266 |
| | LFR1 | DIQMTQSPSSLSASVGDRVTITC | 251 |
| | LFR2 | WYQKKPGKAPKLLIY | 1267 |
| | LFR3 | GVPSRFSGSGSGTEFTFTISSLQPE | 1268 |
| | | DIATYYC | |
| | LFR4 | FGPGTKVDMK | 1269 |
| S626-692 | HC | QVQLVQSGAEVKKPGASVKVSCKAS | 1319 |
| | | GYAFTSYDINWVRQATGQGLEWMGW | |
| | | MNPNSGDTFYAQKFQGRVTMTRSTS | |
| | | ISTAYMELSSLRSEDTAVYYCARGR | |
| | | VGADYVSGNRGYYYYYYMDVWGKGT | |
| | | TVTVSS | |
| | HC | QVQLVQSGAEVKKPGASVKVSCKAS | 1320 |
| | variable | GYAFTSYDINWVRQATGQGLEWMGW | |
| | | MNPNSGDTFYAQKFQGRVTMTRSTS | |
| | | ISTAYMELSSLRSEDTAVYYCARG | |
| | HCDR1 | SYDIN | 1321 |
| | HCDR2 | WMNPNSGDTFYAQKFQG | 1322 |
| | HCDR3 | GRVGADYVSGNRGYYYYYYMDV | 1323 |
| | HFR1 | QVQLVQSGAEVKKPGASVKVSCKAS | 1324 |
| | | GYAFT | |
| | HFR2 | WVRQATGQGLEWMG | 1325 |
| | HFR3 | RVTMTRSTSISTAYMELSSLRSEDT | 1326 |
| | | AVYYCAR | |
| | HFR4 | WGKGTTVTVSS | 670 |
| | LC | SSELTQDPAVSVALGQTVRITCQGE | 1327 |
| | | NLRSYYATWYQQKPGQAPILVIYGK | |
| | | NNRPSGIPDRFSGSSSGNTASLTIT | |
| | | GAQAEDEADYYCNSRDSSGNHLRVF | |
| | | GGGTKLTVL | |
| | LC | SSELTQDPAVSVALGQTVRITCQGE | 1328 |
| | variable | NLRSYYATWYQQKPGQAPILVIYGK | |
| | | NNRPSGIPDRFSGSSSGNTASLTIT | |
| | | GAQAEDEADYYCNSRDSSGNHL | |
| | LCDR1 | QGENLRSYYAT | 1329 |
| | LCDR2 | GKNNRPS | 1330 |
| | LCDR3 | NSRDSSGNHLRV | 1331 |
| | LFR1 | SSELTQDPAVSVALGQTVRITC | 1194 |
| | LFR2 | WYQQKPGQAPILVIY | 1332 |
| | LFR3 | GIPDRFSGSSSGNTASLTITGAQAE | 1196 |
| | | DEADYYC | |
| | LFR4 | FGGGTKLTVL | 69 |
| S626-7 | HC | QVQLVQSGAEVKKPGASVKVSCKAS | 1319 |
| | | GYAFTSYDINWVRQATGQGLEWMGW | |
| | | MNPNSGDTFYAQKFQGRVTMTRSTS | |
| | | ISTAYMELSSLRSEDTAVYYCARGR | |
| | | VGADYVSGNRGYYYYYYMDVWGKGT | |
| | | TVTVSS | |
| | HC | QVQLVQSGAEVKKPGASVKVSCKAS | 1320 |
| | variable | GYAFTSYDINWVRQATGQGLEWMGW | |
| | | MNPNSGDTFYAQKFQGRVTMTRSTS | |
| | | ISTAYMELSSLRSEDTAVYYCARG | |
| | HCDR1 | SYDIN | 1321 |
| | HCDR2 | WMNPNSGDTFYAQKFQG | 1322 |
| | HCDR3 | GRVGADYVSGNRGYYYYYYMDV | 1323 |
| | HFR1 | QVQLVQSGAEVKKPGASVKVSCKAS | 1324 |
| | | GYAFT | |
| | HFR2 | WVRQATGQGLEWMG | 1325 |
| | HFR3 | RVTMTRSTSISTAYMELSSLRSEDT | 1326 |
| | | AVYYCAR | |
| | HFR4 | WGKGTTVTVSS | 670 |
| | LC | SSELTQDPAVSVALGQTVRITCQGE | 1327 |
| | | NLRSYYATWYQQKPGQAPILVIYGK | |
| | | NNRPSGIPDRFSGSSSGNTASLTIT | |
| | | GAQAEDEADYYCNSRDSSGNHLRVF | |
| | | GGGTKLTVL | |
| | LC | SSELTQDPAVSVALGQTVRITCQGE | 1328 |
| | variable | NLRSYYATWYQQKPGQAPILVIYGK | |
| | | NNRPSGIPDRFSGSSSGNTASLTIT | |
| | | GAQAEDEADYYCNSRDSSGNHL | |
| | LCDR1 | QGENLRSYYAT | 1329 |
| | LCDR2 | GKNNRPS | 1330 |
| | LCDR3 | NSRDSSGNHLRV | 1331 |
| | LFR1 | SSELTQDPAVSVALGQTVRITC | 1194 |
| | LFR2 | WYQQKPGQAPILVIY | 1332 |
| | LFR3 | GIPDRFSGSSSGNTASLTITGAQAE | 1196 |
| | | DEADYYC | |
| | LFR4 | FGGGTKLTVL | 69 |
| S626-747 | HC | EPQLVESGGGLVQPGGSLRLSCAGS | 1270 |
| | | GFGFISYPMNWVRQAPGKGPEWISN | |
| | | IRTTAEGGTFYADSVKGRFTMSRDD | |
| | | GKTSIYLQMNSLRDEDTATYYCARD | |
| | | SSYGFDLWGQGTVVTVSS | |
| | HC | EPQLVESGGGLVQPGGSLRLSCAGS | 1271 |
| | variable | GFGFISYPMNWVRQAPGKGPEWISN | |
| | | IRTTAEGGTFYADSVKGRFTMSRDD | |
| | | GKTSIYLQMNSLRDEDTATYYCAR | |
| | HCDR1 | SYPMN | 1272 |
| | HCDR2 | NIRTTAEGGTFYADSVKG | 1273 |
| | HCDR3 | DSSYGFDL | 1274 |
| | HFR1 | EPQLVESGGGLVQPGGSLRLSCAGS | 1275 |
| | | GFGFI | |
| | HFR2 | WVRQAPGKGPEWIS | 1276 |
| | HFR3 | RFTMSRDDGKTSIYLQMNSLRDEDT | 1277 |
| | | ATYYCAR | |
| | HFR4 | WGQGTVVTVSS | 421 |
| | LC | QSALTQPRSVSGSPGQSVTISCTGT | 1278 |
| | | SSDVGGYNYVSWYQQRPGKAPELMI | |
| | | YHVSERPSGVPDRFSGSKSGNTASL | |
| | | TISRLQAEDEADYYCCSYAGSHFWV | |
| | | FGGGTKLTVL | |
| | LC | QSALTQPRSVSGSPGQSVTISCTGT | 1279 |
| | variable | SSDVGGYNYVSWYQQRPGKAPELMI | |
| | | YHVSERPSGVPDRFSGSKSGNTASL | |
| | | TISRLQAEDEADYYCCSYAGSH | |
| | LCDR1 | TGTSSDVGGYNYVS | 63 |
| | LCDR2 | HVSERPS | 1280 |
| | LCDR3 | CSYAGSHFWV | 1281 |
| | LFR1 | QSALTQPRSVSGSPGQSVTISC | 594 |
| | LFR2 | WYQQRPGKAPELMIY | 1282 |
| | LFR3 | GVPDRFSGSKSGNTASLTISRLQAE | 1283 |
| | | DEADYYC | |
| | LFR4 | FGGGTKLTVL | 69 |
| S626-75 | HC | QVQLQESGPGLVKPSQTLSLTCTVS | 1284 |
| | | GGSISSGDYYWSWIRQPPGKGLEWL | |
| | | GYIYYSGSTFYNPSLKSRVTISVDT | |
| | | SKNQFSLRLTSVTAADTAVYFCARE | |
| | | ENKFNYGHHPLNGVFAYWGQGTLVT | |
| | | VSS | |
| | HC | QVQLQESGPGLVKPSQTLSLTCTVS | 1285 |
| | variable | GGSISSGDYYWSWIRQPPGKGLEWL | |
| | | GYIYYSGSTFYNPSLKSRVTISVDT | |
| | | SKNQFSLRLTSVTAADTAVYFCAR | |
| | HCDR1 | SGDYYWS | 72 |
| | HCDR2 | YIYYSGSTFYNPSLKS | 1286 |
| | HCDR3 | EENKFNYGHHPLNGVFAY | 1287 |
| | HFR1 | QVQLQESGPGLVKPSQTLSLTCTVS | 1288 |
| | | GGSIS | |
| | HFR2 | WIRQPPGKGLEWLG | 1289 |
| | HFR3 | RVTISVDTSKNQFSLRLTSVTAADT | 1290 |
| | | AVYFCAR | |
| | HFR4 | WGQGTLVTVSS | 60 |
| | LC | DIVMTQSPDSLAVSLGERATINCKS | 1291 |
| | | SQSVLYSPNNKNYLAWYQQKPGQPP | |
| | | NLLIYWASTRESGVPDRFSGSGSGT | |
| | | DFTLTINSLQAEDVAVYYCQQSYNT | |
| | | PRTFGQGTKVEIK | |
| | LC | DIVMTQSPDSLAVSLGERATINCKS | 1292 |
| | variable | SQSVLYSPNNKNYLAWYQQKPGQPP | |
| | | NLLIYWASTRESGVPDRFSGSGSGT | |
| | | DFTLTINSLQAEDVAVYYCQQSYNT | |
| | | P | |
| | LCDR1 | KSSQSVLYSPNNKNYLA | 1293 |
| | LCDR2 | WASTRES | 30 |
| | LCDR3 | QQSYNTPRT | 1294 |
| | LFR1 | DIVMTQSPDSLAVSLGERATINC | 32 |
| | LFR2 | WYQQKPGQPPNLLIY | 1295 |
| | LFR3 | GVPDRFSGSGSGTDFTLTINSLQAE | 1296 |
| | | DVAVYYC | |
| | LFR4 | FGQGTKVEIK | 53 |
| S626-8 | HC | QVQLVESGGGVVQPGRSLRLSCVSS | 1333 |
| | | EVTFNRYTMHWVRQAPGKGLEWVAS | |
| | | ISFEGSVKTYVDSVKGRFTISRDDS | |
| | | KKTLFLQLNSLRDEDTAMYYCARGQ | |
| | | WPSGGDYWGRGTLVTVSS | |
| | HC | QVQLVESGGGVVQPGRSLRLSCVSS | 1334 |
| | variable | EVTFNRYTMHWVRQAPGKGLEWVAS | |
| | | ISFEGSVKTYVDSVKGRFTISRDDS | |
| | | KKTLFLQLNSLRDEDTAMYYCAR | |
| | HCDR1 | RYTMH | 1335 |
| | HCDR2 | SISFEGSVKTYVDSVKG | 1336 |
| | HCDR3 | GQWPSGGDY | 1337 |
| | HFR1 | QVQLVESGGGVVQPGRSLRLSCVSS | 1338 |
| | | EVTFN | |
| | HFR2 | WVRQAPGKGLEWVA | 145 |
| | HFR3 | RFTISRDDSKKTLFLQLNSLRDEDT | 1339 |
| | | AMYYCAR | |
| | HFR4 | WGRGTLVTVSS | 9 |
| | LC | DVVLTQSPLSLSVTLGQPASISCRS | 1340 |
| | | SQSLVYSDGSTYLNWFHQRPGQSPR | |
| | | RLIYKVSNRDSGVPDRFSGSGSGTD | |
| | | FTLKITRVAAEDVGVYYCMQGTYWP | |
| | | PTFGQGTKVEIK | |
| | LC | DVVLTQSPLSLSVTLGQPASISCRS | 134 |
| | variable | SQSLVYSDGSTYLNWFHQRPGQSPR | |
| | | RLIYKVSNRDSGVPDRFSGSGSGTD | |
| | | FTLKITRVAAEDVGVYYCMQGTYWP | |
| | | P | |
| | LCDR1 | RSSQSLVYSDGSTYLN | 1342 |
| | LCDR2 | KVSNRDS | 1343 |
| | LCDR3 | MQGTYWPPT | 1344 |
| | LFR1 | DVVLTQSPLSLSVTLGQPASISC | 1345 |
| | LFR2 | WFHQRPGQSPRRLIY | 1346 |
| | LFR3 | GVPDRFSGSGSGTDFTLKITRVAAE | 1347 |
| | | DVGVYYC | |
| | LFR4 | FGQGTKVEIK | 53 |
| S68-253 | HC | EVQLVQSGAEIKKPGESLKISCQGS | 1348 |
| | | GYIFTNNWIGWVRQQPGKGLEWMGI | |
| | | IYPGDSDARYSPSFQGHVSFSADKS | |
| | | INTAFLQWHSLKASDTAMYYCARIR | |
| | | RRGQGATAAFDIWGPGTKVTVSS | |
| | HC | EVQLVQSGAEIKKPGESLKISCQGS | 1349 |
| | variable | GYIFTNNWIGWVRQQPGKGLEWMGI | |
| | | IYPGDSDARYSPSFQGHVSFSADKS | |
| | | INTAFLQWHSLKASDTAMYYCAR | |
| | HCDR1 | NNWIG | 1350 |
| | HCDR2 | IIYPGDSDARYSPSFQG | 1351 |
| | HCDR3 | IRRRGQGATAAFDI | 1352 |
| | HFR1 | EVQLVQSGAEIKKPGESLKISCQGS | 1353 |
| | | GYIFT | |
| | HFR2 | WVRQQPGKGLEWMG | 1354 |
| | HFR3 | HVSFSADKSINTAFLQWHSLKASDT | 1355 |
| | | AMYYCAR | |
| | HFR4 | WGPGTKVTVSS | 1356 |
| | LC | DIVMTQSPDSLTVSLGERATINCKS | 1357 |
| | | SQNILTTSNNKNYLAWYQQKPGQPP | |
| | | KLLIYWASTRESGVPDRFSGSGSGT | |
| | | DFTLTISSLQAEDVAVYYCQQYFNS | |
| | | PPYTFGQGTKLEIK | |
| | LC | DIVMTQSPDSLTVSLGERATINCKS | 1358 |
| | variable | SQNILTTSNNKNYLAWYQQKPGQPP | |
| | | KLLIYWASTRESGVPDRFSGSGSGT | |
| | | DFTLTISSLQAEDVAVYYCQQYFNS | |
| | | PP | |
| | LCDR1 | KSSQNILTTSNNKNYLA | 1359 |
| | LCDR2 | WASTRES | 30 |
| | LCDR3 | QQYFNSPPYT | 1360 |
| | LFR1 | DIVMTQSPDSLTVSLGERATINC | 925 |
| | LFR2 | WYQQKPGQPPKLLIY | 33 |
| | LFR3 | GVPDRFSGSGSGTDFTLTISSLQAE | 293 |
| | | DVAVYYC | |
| | LFR4 | FGQGTKLEIK | 380 |
| S728-1502 | HC | QVQLVQSGAEVKKPGASVKVSCKAS | 136 |
| | | GYTFTGYYMHWVRQAPGQGLEWMGR | |
| | | INPNSGGTNYAQKFQGRVTMTRDTS | |
| | | ISTAYMELSRLRSDDTAVYYCARAH | |
| | | QPLLYGLGYYFDYWGQGTLVTVSS | |
| | HC | QVQLVQSGAEVKKPGASVKVSCKAS | 1362 |
| | variable | GYTFTGYYMHWVRQAPGQGLEWMGR | |
| | | INPNSGGTNYAQKFQGRVTMTRDTS | |
| | | ISTAYMELSRLRSDDTAVYYCAR | |
| | HCDR1 | GYYMH | 187 |
| | HCDR2 | RINPNSGGTNYAQKFQG | 585 |
| | HCDR3 | AHQPLLYGLGYYFDY | 1363 |
| | HFR1 | QVQLVQSGAEVKKPGASVKVSCKAS | 190 |
| | | GYTFT | |
| | HFR2 | WVRQAPGQGLEWMG | 160 |
| | HFR3 | RVTMTRDTSISTAYMELSRLRSDDT | 588 |
| | | AVYYCAR | |
| | HFR4 | WGQGTLVTVSS | 60 |
| | LC | QSALTQPASVSGSPGQSITISCTGT | 1364 |
| | | SSDVGGYNYVSWYQQHPGKAPKLMI | |
| | | YEVSNRPSGVSNRFSGSKSGNTASL | |
| | | TISGLQAEDEADYYCSSYTSSSTLV | |
| | | FGGGTKLTVL | |
| | LC | QSALTQPASVSGSPGQSITISCTGT | 1365 |
| | variable | SSDVGGYNYVSWYQQHPGKAPKLMI | |
| | | YEVSNRPSGVSNRFSGSKSGNTASL | |
| | | TISGLQAEDEADYYCSSYTSSSTL | |
| | LCDR1 | TGTSSDVGGYNYVS | 63 |
| | LCDR2 | EVSNRPS | 151 |
| | LCDR3 | SSYTSSSTLV | 1366 |
| | LFR1 | QSALTQPASVSGSPGQSITISC | 66 |
| | LFR2 | WYQQHPGKAPKLMIY | 67 |
| | LFR3 | GVSNRFSGSKSGNTASLTISGLQAE | 68 |
| | | DEADYYC | |
| | LFR4 | FGGGTKLTVL | 69 |
| S728-1789 | HC | EVQLVESGGGLVQPGGSLRLSCAAS | 1367 |
| | | GFTFSSYWMHWVRQAPGKGLVWVSR | |
| | | INSDGSSTSYADSVKGRFTISRDNA | |
| | | KNTLYLQMNSLRAEDTAVYYCARDR | |
| | | YSSLDYWGQGTLVTVSS | |
| | HC | EVQLVESGGGLVQPGGSLRLSCAAS | 1368 |
| | variable | GFTFSSYWMHWVRQAPGKGLVWVSR | |
| | | INSDGSSTSYADSVKGRFTISRDNA | |
| | | KNTLYLQMNSLRAEDTAVYYCAR | |
| | HCDR1 | SYWMH | 1369 |
| | HCDR2 | RINSDGSSTSYADSVKG | 1370 |
| | HCDR3 | DRYSSLDY | 1371 |
| | HFR1 | EVQLVESGGGLVQPGGSLRLSCAAS | 613 |
| | | GFTFS | |
| | HFR2 | WVRQAPGKGLVWVS | 1372 |
| | HFR3 | RFTISRDNAKNTLYLQMNSLRAEDT | 1373 |
| | | AVYYCAR | |
| | HFR4 | WGQGTLVTVSS | 60 |
| | LC | EIVLTQSPGTLSLSPGERATLSCRA | 1374 |
| | | SQSVSSSYLAWYQQKPGQAPRLLIY | |
| | | GASSRATGIPDRFSGSGSGTDFTLT | |
| | | ISRLEPEDFAVYYCQQYGSSPALTF | |
| | | GGGTKVEIK | |
| | LC | EIVLTQSPGTLSLSPGERATLSCRA | 1201 |
| | variable | SQSVSSSYLAWYQQKPGQAPRLLIY | |
| | | GASSRATGIPDRFSGSGSGTDFTLT | |
| | | ISRLEPEDFAVYYCQQYGSSP | |
| | LCDR1 | RASQSVSSSYLA | 338 |
| | LCDR2 | GASSRAT | 135 |
| | LCDR3 | QQYGSSPALT | 1375 |
| | LFR1 | EIVLTQSPGTLSLSPGERATLSC | 50 |
| | LFR2 | WYQQKPGQAPRLLIY | 182 |
| | LFR3 | GIPDRFSGSGSGTDFTLTISRLEPE | 196 |
| | | DFAVYYC | |
| | LFR4 | FGGGTKVEIK | 85 |
| S728-1806 | HC | QVQLVQSGAEVKKPGASVKVSCKAS | 1361 |
| | | GYTFTGYYMHWVRQAPGQGLEWMGR | |
| | | INPNSGGTNYAQKFQGRVTMTRDTS | |
| | | ISTAYMELSRLRSDDTAVYYCARAH | |
| | | QPLLYGLGYYFDYWGQGTLVTVSS | |
| | HC | QVQLVQSGAEVKKPGASVKVSCKAS | 1362 |
| | variable | GYTFTGYYMHWVRQAPGQGLEWMGR | |
| | | INPNSGGTNYAQKFQGRVTMTRDTS | |
| | | ISTAYMELSRLRSDDTAVYYCAR | |
| | HCDR1 | GYYMH | 187 |
| | HCDR2 | RINPNSGGTNYAQKFQG | 585 |
| | HCDR3 | AHQPLLYGLGYYFDY | 1363 |
| | HFR1 | QVQLVQSGAEVKKPGASVKVSCKAS | 190 |
| | | GYTFT | |
| | HFR2 | WVRQAPGQGLEWMG | 160 |
| | HFR3 | RVTMTRDTSISTAYMELSRLRSDDT | 588 |
| | | AVYYCAR | |
| | HFR4 | WGQGTLVTVSS | 60 |
| | LC | QSALTQPASVSGSPGQSITISCTGT | 1364 |
| | | SSDVGGYNYVSWYQQHPGKAPKLMI | |
| | | YEVSNRPSGVSNRFSGSKSGNTASL | |
| | | TISGLQAEDEADYYCSSYTSSSTLV | |
| | | FGGGTKLTVL | |
| | LC | QSALTQPASVSGSPGQSITISCTGT | 1365 |
| | variable | SSDVGGYNYVSWYQQHPGKAPKLMI | |
| | | YEVSNRPSGVSNRFSGSKSGNTASL | |
| | | TISGLQAEDEADYYCSSYTSSSTL | |
| | LCDR1 | TGTSSDVGGYNYVS | 63 |
| | LCDR2 | EVSNRPS | 151 |
| | LCDR3 | SSYTSSSTLV | 1366 |
| | LFR1 | QSALTQPASVSGSPGQSITISC | 66 |
| | LFR2 | WYQQHPGKAPKLMIY | 67 |
| | LFR3 | GVSNRFSGSKSGNTASLTISGLQAE | 68 |
| | | DEADYYC | |
| | LFR4 | FGGGTKLTVL | 69 |
| S728-1981 | HC | QVQLVQSGAEVKKPGASVKVSCKTS | 1376 |
| | | GYTFTNYFMHWVRQAPGQGLEWMGI | |
| | | INPSGGSASYAQKFQGRITMTSDTS | |
| | | TSTVYMELSSLRSEDTAVYYCARED | |
| | | IIVVVPARPLDYWGHGTLVTVSS | |
| | HC | QVQLVQSGAEVKKPGASVKVSCKTS | 1377 |
| | variable | GYTFTNYFMHWVRQAPGQGLEWMGI | |
| | | INPSGGSASYAQKFQGRITMTSDTS | |
| | | TSTVYMELSSLRSEDTAVYYCAR | |
| | HCDR1 | NYFMH | 1378 |
| | HCDR2 | IINPSGGSASYAQKFQG | 1379 |
| | HCDR3 | EDIIVVVPARPLDY | 1380 |
| | HFR1 | QVQLVQSGAEVKKPGASVKVSCKTS | 1381 |
| | | GYTFT | |
| | HFR2 | WVRQAPGQGLEWMG | 160 |
| | HFR3 | RITMTSDTSTSTVYMELSSLRSEDT | 1382 |
| | | AVYYCAR | |
| | HFR4 | WGHGTLVTVSS | 1315 |
| | LC | EIVLTQSPATLSLSPGERATLSCRA | 1383 |
| | | SQSFSNYLAWYQQKPGQAPRLLIYD | |
| | | ASNRATGIPARFSGSGSGTDFTLTI | |
| | | SSLEPEDFAVYYCQQRSNWPPLLTF | |
| | | GGGTKVEIK | |
| | LC | EIVLTQSPATLSLSPGERATLSCRA | 1384 |
| | variable | SQSFSNYLAWYQQKPGQAPRLLIYD | |
| | | ASNRATGIPARFSGSGSGTDFTLTI | |
| | | SSLEPEDFAVYYCQQRSNWPP | |
| | LCDR1 | RASQSFSNYLA | 1385 |
| | LCDR2 | DASNRAT | 441 |
| | LCDR3 | QQRSNWPPLLT | 1386 |
| | LFR1 | EIVLTQSPATLSLSPGERATLSC | 181 |
| | LFR2 | WYQQKPGQAPRLLIY | 182 |
| | LFR3 | GIPARFSGSGSGTDFTLTISSLEPE | 183 |
| | | DFAVYYC | |
| | LFR4 | FGGGTKVEIK | 85 |
| S728-2036 | HC | QVHLVQSGAEIRKPGASVMVSCKAS | 1387 |
| | | GYTFTDYYIHWVRQAPGQGLEWMGW | |
| | | INPNSGGTNYAQKFQGRVTMTRDTS | |
| | | IRTAYMELSRLRSDDAAVYYCAREG | |
| | | ISMLRGVRSWFDPWGQGTLVTVSS | |
| | HC | QVHLVQSGAEIRKPGASVMVSCKAS | 1388 |
| | variable | GYTFTDYYIHWVRQAPGQGLEWMGW | |
| | | INPNSGGTNYAQKFQGRVTMTRDTS | |
| | | IRTAYMELSRLRSDDAAVYYCAR | |
| | HCDR1 | DYYIH | 1389 |
| | HCDR2 | WINPNSGGTNYAQKFQG | 953 |
| | HCDR3 | EGISMLRGVRSWFDP | 1390 |
| | HFR1 | QVHLVQSGAEIRKPGASVMVSCKAS | 1391 |
| | | GYTFT | |
| | HFR2 | WVRQAPGQGLEWMG | 160 |
| | HFR3 | RVTMTRDTSIRTAYMELSRLRSDDA | 1392 |
| | | AVYYCAR | |
| | HFR4 | WGQGTLVTVSS | 60 |
| | LC | QSALTQPASVSGSPGQSITISCTGT | 1393 |
| | | SSDVGSYNLVSWYQQHPGKVPKLII | |
| | | YEVTKRPSGVSNRFSGSKSGNTASL | |
| | | TISGLQTEDEADYYCCSYAGFSAWV | |
| | | FGGGTKLTVL | |
| | LC | QSALTQPASVSGSPGQSITISCTGT | 1394 |
| | variable | SSDVGSYNLVSWYQQHPGKVPKLII | |
| | | YEVTKRPSGVSNRFSGSKSGNTASL | |
| | | TISGLQTEDEADYYCCSYAGFSA | |
| | LCDR1 | TGTSSDVGSYNLVS | 1395 |
| | LCDR2 | EVTKRPS | 1396 |
| | LCDR3 | CSYAGFSAWV | 1397 |
| | LFR1 | QSALTQPASVSGSPGQSITISC | 66 |
| | LFR2 | WYQQHPGKVPKLIIY | 1398 |
| | LFR3 | GVSNRFSGSKSGNTASLTISGLQTE | 1399 |
| | | DEADYYC | |
| | LFR4 | FGGGTKLTVL | 69 |
| S728-2111 | HC | EVQLVESGGGLVQPGGSLRLSCAAS | 1367 |
| | | GFTFSSYWMHWVRQAPGKGLVWVSR | |
| | | INSDGSSTSYADSVKGRFTISRDNA | |
| | | KNTLYLQMNSLRAEDTAVYYCARDR | |
| | | YSSLDYWGQGTLVTVSS | |
| | HC | EVQLVESGGGLVQPGGSLRLSCAAS | 1368 |
| | variable | GFTFSSYWMHWVRQAPGKGLVWVSR | |
| | | INSDGSSTSYADSVKGRFTISRDNA | |
| | | KNTLYLQMNSLRAEDTAVYYCAR | |
| | HCDR1 | SYWMH | 1369 |
| | HCDR2 | RINSDGSSTSYADSVKG | 1370 |
| | HCDR3 | DRYSSLDY | 1371 |
| | HFR1 | EVQLVESGGGLVQPGGSLRLSCAAS | 613 |
| | | GFTFS | |
| | HFR2 | WVRQAPGKGLVWVS | 1372 |
| | HFR3 | RFTISRDNAKNTLYLQMNSLRAEDT | 1373 |
| | | AVYYCAR | |
| | HFR4 | WGQGTLVTVSS | 60 |
| | LC | EIVLTQSPGTLSLSPGERATLSCRA | 1374 |
| | | SQSVSSSYLAWYQQKPGQAPRLLIY | |
| | | GASSRATGIPDRFSGSGSGTDFTLT | |
| | | ISRLEPEDFAVYYCQQYGSSPALTF | |
| | | GGGTKVEIK | |
| | LC | EIVLTQSPGTLSLSPGERATLSCRA | 1201 |
| | variable | SQSVSSSYLAWYQQKPGQAPRLLIY | |
| | | GASSRATGIPDRFSGSGSGTDFTLT | |
| | | ISRLEPEDFAVYYCQQYGSSP | |
| | LCDR1 | RASQSVSSSYLA | 338 |
| | LCDR2 | GASSRAT | 135 |
| | LCDR3 | QQYGSSPALT | 1375 |
| | LFR1 | EIVLTQSPGTLSLSPGERATLSC | 50 |
| | LFR2 | WYQQKPGQAPRLLIY | 182 |
| | LFR3 | GIPDRFSGSGSGTDFTLTISRLEPE | 196 |
| | | DFAVYYC | |
| | LFR4 | FGGGTKVEIK | 85 |
| S728-2148 | HC | QVQLVESGGGVVQPGRSLRLSCAAS | 1147 |
| | | GFTFSSYGMHWVRQAPGKGLEWVAV | |
| | | IWYDGSNKYYADSVKGRFTISRDNS | |
| | | KNTLYLQMNSLRAEDTAVYYCARAP | |
| | | DYWGQGTLVTVSS | |
| | HC | QVQLVESGGGVVQPGRSLRLSCAAS | 1148 |
| | variable | GFTFSSYGMHWVRQAPGKGLEWVAV | |
| | | IWYDGSNKYYADSVKGRFTISRDNS | |
| | | KNTLYLQMNSLRAEDTAVYYCAR | |
| | HCDR1 | SYGMH | 141 |
| | HCDR2 | VIWYDGSNKYYADSVKG | 142 |
| | HCDR3 | APDY | 1149 |
| | HFR1 | QVQLVESGGGVVQPGRSLRLSCAAS | 144 |
| | | GFTFS | |
| | HFR2 | WVRQAPGKGLEWVA | 145 |
| | HFR3 | RFTISRDNSKNTLYLQMNSLRAEDT | 146 |
| | | AVYYCAR | |
| | HFR4 | WGQGTLVTVSS | 60 |
| | LC | AIQMTQSPSSLSASVGDRVTITCRA | 1150 |
| | | SQGIRNDLGWYQQKPGKAPKLLIYA | |
| | | ASSLQSGVPSRFSGSGSGTDFTLTI | |
| | | SSLQPEDFATYYCLQDYNYPYTFGQ | |
| | | GTKLEIK | |
| | LC | AIQMTQSPSSLSASVGDRVTITCRA | 1151 |
| | variable | SQGIRNDLGWYQQKPGKAPKLLIYA | |
| | | ASSLQSGVPSRFSGSGSGTDFTLTI | |
| | | SSLQPEDFATYYCLQDYNYP | |
| | LCDR1 | RASQGIRNDLG | 1152 |
| | LCDR2 | AASSLQS | 249 |
| | LCDR3 | LQDYNYPYT | 1153 |
| | LFR1 | AIQMTQSPSSLSASVGDRVTITC | 1154 |
| | LFR2 | WYQQKPGKAPKLLIY | 252 |
| | LFR3 | GVPSRFSGSGSGTDFTLTISSLQPE | 253 |
| | | DFATYYC | |
| | LFR4 | FGQGTKLEIK | 380 |
| S728-656 | HC | EVQLLESGGGLVQPGGSLRLSCAAS | 1400 |
| | | GFTFSSYVLSWVRQAPGKGLEWVSA | |
| | | ISGSGGITYYADSVKGRFTISRDNS | |
| | | KNTLYLQMNSLRAEDTAVYYCAIRI | |
| | | TISGVFTPAWDSWGQGTLVTVSS | |
| | HC | EVQLLESGGGLVQPGGSLRLSCAAS | 1401 |
| | variable | GFTFSSYVLSWVRQAPGKGLEWVSA | |
| | | ISGSGGITYYADSVKGRFTISRDNS | |
| | | KNTLYLQMNSLRAEDTAVYYCA | |
| | HCDR1 | SYVLS | 1402 |
| | HCDR2 | AISGSGGITYYADSVKG | 1403 |
| | HCDR3 | RITISGVFTPAWDS | 1404 |
| | HFR1 | EVQLLESGGGLVQPGGSLRLSCAAS | 566 |
| | | GFTFS | |
| | HFR2 | WVRQAPGKGLEWVS | 130 |
| | HFR3 | RFTISRDNSKNTLYLQMNSLRAEDT | 1405 |
| | | AVYYCAI | |
| | HFR4 | WGQGTLVTVSS | 60 |
| | LC | DIQMTQSPSSLSASVGDRVTITCRA | 1406 |
| | | SQSISTYLNWYQQKPGKAPKLLIYA | |
| | | ASSLQSGVPSRFSGSGSGTDFTLTI | |
| | | SSLQPEDFATYFCQQSYSSPFTFGP | |
| | | GTKVDIK | |
| | LC | DIQMTQSPSSLSASVGDRVTITCRA | 1407 |
| | variable | SQSISTYLNWYQQKPGKAPKLLIYA | |
| | | ASSLQSGVPSRFSGSGSGTDFTLTI | |
| | | SSLQPEDFATYFCQQSYSSP | |
| | LCDR1 | RASQSISTYLN | 1036 |
| | LCDR2 | AASSLQS | 249 |
| | LCDR3 | QQSYSSPFT | 1408 |
| | LFR1 | DIQMTQSPSSLSASVGDRVTITC | 251 |
| | LFR2 | WYQQKPGKAPKLLIY | 252 |
| | LFR3 | GVPSRFSGSGSGTDFTLTISSLQPE | 1409 |
| | | DFATYFC | |
| | LFR4 | FGPGTKVDIK | 443 |
| S728-723 | HC | QVQLVQSGAEVKKPGASVKVSCKTS | 1376 |
| | | GYTFTNYFMHWVRQAPGQGLEWMGI | |
| | | INPSGGSASYAQKFQGRITMTSDTS | |
| | | TSTVYMELSSLRSEDTAVYYCARED | |
| | | IIVVVPARPLDYWGHGTLVTVSS | |
| | HC | QVQLVQSGAEVKKPGASVKVSCKTS | 1377 |
| | variable | GYTFTNYFMHWVRQAPGQGLEWMGI | |
| | | INPSGGSASYAQKFQGRITMTSDTS | |
| | | TSTVYMELSSLRSEDTAVYYCAR | |
| | HCDR1 | NYFMH | 1378 |
| | HCDR2 | IINPSGGSASYAQKFQG | 1379 |
| | HCDR3 | EDIIVVVPARPLDY | 1380 |
| | HFR1 | QVQLVQSGAEVKKPGASVKVSCKTS | 1381 |
| | | GYTFT | |
| | HFR2 | WVRQAPGQGLEWMG | 160 |
| | HFR3 | RITMTSDTSTSTVYMELSSLRSEDT | 1382 |
| | | AVYYCAR | |
| | HFR4 | WGHGTLVTVSS | 1315 |
| | LC | EIVLTQSPATLSLSPGERATLSCRA | 1383 |
| | | SQSFSNYLAWYQQKPGQAPRLLIYD | |
| | | ASNRATGIPARFSGSGSGTDFTLTI | |
| | | SSLEPEDFAVYYCQQRSNWPPLLTF | |
| | | GGGTKVEIK | |
| | LC | EIVLTQSPATLSLSPGERATLSCRA | 1384 |
| | variable | SQSFSNYLAWYQQKPGQAPRLLIYD | |
| | | ASNRATGIPARFSGSGSGTDFTLTI | |
| | | SSLEPEDFAVYYCQQRSNWPP | |
| | LCDR1 | RASQSFSNYLA | 1385 |
| | LCDR2 | DASNRAT | 441 |
| | LCDR3 | QQRSNWPPLLT | 1386 |
| | LFR1 | EIVLTQSPATLSLSPGERATLSC | 181 |
| | LFR2 | WYQQKPGQAPRLLIY | 182 |
| | LFR3 | GIPARFSGSGSGTDFTLTISSLEPE | 183 |
| | | DFAVYYC | |
| | LFR4 | FGGGTKVEIK | 85 |
| S728-826 | HC | EVQLVESGGGLVQPGGSLRLSCAAS | 1182 |
| | | GFTFGSYWMNWVRQAPGKGLEWVAN | |
| | | INEDGSEKYYVDSVKGRFTISRDNA | |
| | | KNSLYLQMNSLRAEDTAVYYCARGH | |
| | | SLGEWGQGSPVTVSS | |
| | HC | EVQLVESGGGLVQPGGSLRLSCAAS | 1183 |
| | variable | GFTFGSYWMNWVRQAPGKGLEWVAN | |
| | | INEDGSEKYYVDSVKGRFTISRDNA | |
| | | KNSLYLQMNSLRAEDTAVYYCAR | |
| | HCDR1 | SYWMN | 1184 |
| | HCDR2 | NINEDGSEKYYVDSVKG | 1185 |
| | HCDR3 | GHSLGE | 1186 |
| | HFR1 | EVQLVESGGGLVQPGGSLRLSCAAS | 1187 |
| | | GFTFG | |
| | HFR2 | WVRQAPGKGLEWVA | 145 |
| | HFR3 | RFTISRDNAKNSLYLQMNSLRAEDT | 273 |
| | | AVYYCAR | |
| | HFR4 | WGQGSPVTVSS | 1188 |
| | LC | SSELTQDPAVSVALGQTVRITCQGD | 1189 |
| | | SLRSYSASWYQQKPGQAPVLVIYIK | |
| | | NKRPSGIPDRFSGSSSGNTASLTIT | |
| | | GAQAEDEADYYCNSRDSSGDHLVFG | |
| | | GGTKLTVL | |
| | LC | SSELTQDPAVSVALGQTVRITCQGD | 1190 |
| | variable | SLRSYSASWYQQKPGQAPVLVIYIK | |
| | | NKRPSGIPDRFSGSSSGNTASLTIT | |
| | | GAQAEDEADYYCNSRDSSGDHL | |
| | LCDR1 | QGDSLRSYSAS | 1191 |
| | LCDR2 | IKNKRPS | 1192 |
| | LCDR3 | NSRDSSGDHLV | 1193 |
| | LFR1 | SSELTQDPAVSVALGQTVRITC | 1194 |
| | LFR2 | WYQQKPGQAPVLVIY | 1195 |
| | LFR3 | GIPDRFSGSSSGNTASLTITGAQAE | 1196 |
| | | DEADYYC | |
| | LFR4 | FGGGTKLTVL | 69 |
| S728-959 | HC | QVHLVQSGAEIRKPGASVMVSCKAS | 1387 |
| | | GYTFTDYYIHWVRQAPGQGLEWMGW | |
| | | INPNSGGTNYAQKFQGRVTMTRDTS | |
| | | IRTAYMELSRLRSDDAAVYYCAREG | |
| | | ISMLRGVRSWFDPWGQGTLVTVSS | |
| | HC | QVHLVQSGAEIRKPGASVMVSCKAS | 1388 |
| | variable | GYTFTDYYIHWVRQAPGQGLEWMGW | |
| | | INPNSGGTNYAQKFQGRVTMTRDTS | |
| | | IRTAYMELSRLRSDDAAVYYCAR | |
| | HCDR1 | DYYIH | 1389 |
| | HCDR2 | WINPNSGGTNYAQKFQG | 953 |
| | HCDR3 | EGISMLRGVRSWFDP | 1390 |
| | HFR1 | QVHLVQSGAEIRKPGASVMVSCKAS | 1391 |
| | | GYTFT | |
| | HFR2 | WVRQAPGQGLEWMG | 160 |
| | HFR3 | RVTMTRDTSIRTAYMELSRLRSDDA | 1392 |
| | | AVYYCAR | |
| | HFR4 | WGQGTLVTVSS | 60 |
| | LC | QSALTQPASVSGSPGQSITISCTGT | 1393 |
| | | SSDVGSYNLVSWYQQHPGKVPKLII | |
| | | YEVTKRPSGVSNRFSGSKSGNTASL | |
| | | TISGLQTEDEADYYCCSYAGFSAWV | |
| | | FGGGTKLTVL | |
| | LC | QSALTQPASVSGSPGQSITISCTGT | 1394 |
| | variable | SSDVGSYNLVSWYQQHPGKVPKLII | |
| | | YEVTKRPSGVSNRFSGSKSGNTASL | |
| | | TISGLQTEDEADYYCCSYAGFSA | |
| | LCDR1 | TGTSSDVGSYNLVS | 1395 |
| | LCDR2 | EVTKRPS | 1396 |
| | LCDR3 | CSYAGFSAWV | 1397 |
| | LFR1 | QSALTQPASVSGSPGQSITISC | 66 |
| | LFR2 | WYQQHPGKVPKLIIY | 1398 |
| | LFR3 | GVSNRFSGSKSGNTASLTISGLQTE | 1399 |
| | | DEADYYC | |
| | LFR4 | FGGGTKLTVL | 69 |
| S210-530 | HC | QVQLVQSGAEVKKPGASVKVSCKAS | 1410 |
| | | GYTFTGYFIHWVRQAPGQGLEYMGW | |
| | | INPNSAGTNYAQKFQGRVTMTGDTS | |
| | | ISTVYMELSRLRSDDTAMYYCARVF | |
| | | FDWLLPFDYWGQGTLVTVSS | |
| | HC | QVQLVQSGAEVKKPGASVKVSCKAS | 1411 |
| | variable | GYTFTGYFIHWVRQAPGQGLEYMGW | |
| | | INPNSAGTNYAQKFQGRVTMTGDTS | |
| | | ISTVYMELSRLRSDDTAMYYCAR | |
| | HCDR1 | GYFIH | 1412 |
| | HCDR2 | WINPNSAGTNYAQKFQG | 1413 |
| | HCDR3 | VFFDWLLPFDY | 1414 |
| | HFR1 | QVQLVQSGAEVKKPGASVKVSCKAS | 190 |
| | | GYTFT | |
| | HFR2 | WVRQAPGQGLEYMG | 1415 |
| | HFR3 | RVTMTGDTSISTVYMELSRLRSDDT | 1416 |
| | | AMYYCAR | |
| | HFR4 | WGQGTLVTVSS | 60 |
| | LC | QSALTQPASVSGSPGQSITISCTGT | 1417 |
| | | SSDVGSYNLVSWYQQHPGKAPKLMI | |
| | | YEVSKRPSGVSNRFSGSKSGNTASL | |
| | | TISGLQAEDEADYYCCSYAGSNYVF | |
| | | GTGTKVTVL | |
| | LC | QSALTQPASVSGSPGQSITISCTGT | 1418 |
| | variable | SSDVGSYNLVSWYQQHPGKAPKLMI | |
| | | YEVSKRPSGVSNRFSGSKSGNTASL | |
| | | TISGLQAEDEADYYCCSYAGS | |
| | LCDR1 | TGTSSDVGSYNLVS | 1395 |
| | LCDR2 | EVSKRPS | 94 |
| | LCDR3 | CSYAGSNYV | 1419 |
| | LFR1 | QSALTQPASVSGSPGQSITISC | 66 |
| | LFR2 | WYQQHPGKAPKLMIY | 67 |
| | LFR3 | GVSNRFSGSKSGNTASLTISGLQAE | 68 |
| | | DEADYYC | |
| | LFR4 | FGTGTKVTVL | 18 |
| S210-1129 | HC | EVQLLESGGGLVQPGGSLRLSCVAS | 1420 |
| | | RFTFSDYAMSWVRQPPGKGLEWVSS | |
| | | ISGSGGITYYADSVKGRFTISRDNS | |
| | | KNTLYLQIKSLRAEDTAIYYCAKER | |
| | | SNWNYVENFDYWGQGTLVTVSS | |
| | HC | EVQLLESGGGLVQPGGSLRLSCVAS | 1421 |
| | variable | RFTFSDYAMSWVRQPPGKGLEWVSS | |
| | | ISGSGGITYYADSVKGRFTISRDNS | |
| | | KNTLYLQIKSLRAEDTAIYYCAK | |
| | HCDR1 | DYAMS | 199 |
| | HCDR2 | SISGSGGITYYADSVKG | 1422 |
| | HCDR3 | ERSNWNYVENFDY | 1423 |
| | HFR1 | EVQLLESGGGLVQPGGSLRLSCVAS | 1424 |
| | | RFTFS | |
| | HFR2 | WVRQPPGKGLEWVS | 104 |
| | HFR3 | RFTISRDNSKNTLYLQIKSLRAEDT | 1425 |
| | | AIYYCAK | |
| | HFR4 | WGQGTLVTVSS | 60 |
| | LC | QTVVTQEPSLTVSPGGTVTLTCASS | 1426 |
| | | TGTVTSAFFPNWFQQKPGQAPRALI | |
| | | YSTTNKYSWTPARFSGSLLGGKAAL | |
| | | TLSGVQPEDEADYYCLLFYGGARPH | |
| | | VVFGGGTKLTVL | |
| | LC | QTVVTQEPSLTVSPGGTVTLTCASS | 1427 |
| | variable | TGTVTSAFFPNWFQQKPGQAPRALI | |
| | | YSTTNKYSWTPARFSGSLLGGKAAL | |
| | | TLSGVQPEDEADYYCLLFYGGA | |
| | LCDR1 | ASSTGTVTSAFFPN | 1428 |
| | LCDR2 | STTNKYS | 1429 |
| | LCDR3 | LLFYGGARPHVV | 1430 |
| | LFR1 | QTVVTQEPSLTVSPGGTVTLTC | 1431 |
| | LFR2 | WFQQKPGQAPRALIY | 1432 |
| | LFR3 | WTPARFSGSLLGGKAALTLSGVQPE | 1433 |
| | | DEADYYC | |
| | LFR4 | FGGGTKLTVL | 69 |
| S451-5 | HC | QVQLQQWGAGLLKPSETLSLTCAVY | 1434 |
| | | GASVSGYFWSWIRQPPGKGLEWIGE | |
| | | INRFGSTNYNPSLKSRVTLSVDTSR | |
| | | NQFSLKLGSVTAADTAMYYCARGSQ | |
| | | ANPLVRFFDSPVTAFDIWGQGTMVT | |
| | | VSS | |
| | HC | QVQLQQWGAGLLKPSETLSLTCAVY | 1435 |
| | variable | GASVSGYFWSWIRQPPGKGLEWIGE | |
| | | INRFGSTNYNPSLKSRVTLSVDTSR | |
| | | NQFSLKLGSVTAADTAMYYCARG | |
| | HCDR1 | GYFWS | 798 |
| | HCDR2 | EINRFGSTNYNPSLKS | 1436 |
| | HCDR3 | GSQANPLVRFFDSPVTAFDI | 1437 |
| | HFR1 | QVQLQQWGAGLLKPSETLSLTCAVY | 1438 |
| | | GASVS | |
| | HFR2 | WIRQPPGKGLEWIG | 7 |
| | HFR3 | RVTLSVDTSRNQFSLKLGSVTAADT | 1439 |
| | | AMYYCAR | |
| | HFR4 | WGQGTMVTVSS | 44 |
| | LC | EIVMTQSPATLSVSPGERATLSCRA | 1440 |
| | | SQSIKSNLAWYQQKPGQAPRLLIYG | |
| | | ASTRATGIPARFSGSGSGTEFTLTI | |
| | | SRLQSEDFALYYCQQYDNWPPYTFG | |
| | | QGTKLEIK | |
| | LC | EIVMTQSPATLSVSPGERATLSCRA | 1441 |
| | variable | SQSIKSNLAWYQQKPGQAPRLLIYG | |
| | | ASTRATGIPARFSGSGSGTEFTLTI | |
| | | SRLQSEDFALYYCQQYDNWPP | |
| | LCDR1 | RASQSIKSNLA | 1442 |
| | LCDR2 | GASTRAT | 208 |
| | LCDR3 | QQYDNWPPYT | 1443 |
| | LFR1 | EIVMTQSPATLSVSPGERATLSC | 210 |
| | LFR2 | WYQQKPGQAPRLLIY | 182 |
| | LFR3 | GIPARFSGSGSGTEFTLTISRLQSE | 1444 |
| | | DFALYYC | |
| | LFR4 | FGQGTKLEIK | 380 |
| S451-506 | HC | QITLKESGPTLVKPTQTLTLTCTFS | 1445 |
| | | GFSFTSSGVGVGWIRQPPGKAMEWL | |
| | | ALIYWDDDKRYSPSLKSRLTITKDT | |
| | | SKNQVVLKMTNMDPVDTATYYCARH | |
| | | TVATIVDYWGQGTLVTVSS | |
| | HC | QITLKESGPTLVKPTQTLTLTCTFS | 1446 |
| | variable | GFSFTSSGVGVGWIRQPPGKAMEWL | |
| | | ALIYWDDDKRYSPSLKSRLTITKDT | |
| | | SKNQVVLKMTNMDPVDTATYYCA | |
| | HCDR1 | SSGVGVG | 1447 |
| | HCDR2 | LIYWDDDKRYSPSLKS | 473 |
| | HCDR3 | HTVATIVDY | 1448 |
| | HFR1 | QITLKESGPTLVKPTQTLTLTCTFS | 1449 |
| | | GFSFT | |
| | HFR2 | WIRQPPGKAMEWLA | 1450 |
| | HFR3 | RLTITKDTSKNQVVLKMTNMDPVDT | 1451 |
| | | ATYYCAR | |
| | HFR4 | WGQGTLVTVSS | 60 |
| | LC | QSALTQPASVSGSPGQSITISCTGT | 1452 |
| | | SSDVGGYNYVSWYQQHPGKAPKLMI | |
| | | YEVSNRPSGVPDRFSGSKSGNTASL | |
| | | TISGLQAEDEADYYCGSYTTSSTPV | |
| | | VFGGGTKLTVL | |
| | LC | QSALTQPASVSGSPGQSITISCTGT | 1453 |
| | variable | SSDVGGYNYVSWYQQHPGKAPKLMI | |
| | | YEVSNRPSGVPDRFSGSKSGNTASL | |
| | | TISGLQAEDEADYYCGSYTTSST | |
| | LCDR1 | TGTSSDVGGYNYVS | 63 |
| | LCDR2 | EVSNRPS | 151 |
| | LCDR3 | GSYTTSSTPVV | 1454 |
| | LFR1 | QSALTQPASVSGSPGQSITISC | 66 |
| | LFR2 | WYQQHPGKAPKLMIY | 67 |
| | LFR3 | GVPDRFSGSKSGNTASLTISGLQAE | 1246 |
| | | DEADYYC | |
| | LFR4 | FGGGTKLTVL | 69 |
| S451-1140 | HC | EVQLVETGGGLIQPGGSLRLSCAAS | 1455 |
| | | GITVSSNYMNWVRLAPGKGLEWVSL | |
| | | IYSGGSTFYADSVKGRFTISRDNSK | |
| | | NTLYLQMNSLRAEDTAVYYCAREGL | |
| | | VGATTAFDYWGQGTLVTVSS | |
| | HC | EVQLVETGGGLIQPGGSLRLSCAAS | 1456 |
| | variable | GITVSSNYMNWVRLAPGKGLEWVSL | |
| | | IYSGGSTFYADSVKGRFTISRDNSK | |
| | | NTLYLQMNSLRAEDTAVYYCAR | |
| | HCDR1 | SNYMN | 1457 |
| | HCDR2 | LIYSGGSTFYADSVKG | 1458 |
| | HCDR3 | EGLVGATTAFDY | 1459 |
| | HFR1 | EVQLVETGGGLIQPGGSLRLSCAAS | 1460 |
| | | GITVS | |
| | HFR2 | WVRLAPGKGLEWVS | 1461 |
| | HFR3 | RFTISRDNSKNTLYLQMNSLRAEDT | 146 |
| | | AVYYCAR | |
| | HFR4 | WGQGTLVTVSS | 60 |
| | LC | DIQLTQSPSFLSASVGDRVTITCRA | 1462 |
| | | SQGISSYLAWYQLQPGKAPKLLIYA | |
| | | ASTLQSGVPSRFSGSGSGTEFTLTI | |
| | | SSLQPEDFATYYCQQLNGHPQGTFG | |
| | | QGTKVEIK | |
| | LC | DIQLTQSPSFLSASVGDRVTITCRA | 1463 |
| | variable | SQGISSYLAWYQLQPGKAPKLLIYA | |
| | | ASTLQSGVPSRFSGSGSGTEFTLTI | |
| | | SSLQPEDFATYYCQQLNGHP | |
| | LCDR1 | RASQGISSYLA | 1464 |
| | LCDR2 | AASTLQS | 1465 |
| | LCDR3 | QQLNGHPQGT | 1466 |
| | LFR1 | DIQLTQSPSFLSASVGDRVTITC | 1467 |
| | LFR2 | WYQLQPGKAPKLLIY | 1468 |
| | LFR3 | GVPSRFSGSGSGTEFTLTISSLQPE | 1469 |
| | | DFATYYC | |
| | LFR4 | FGQGTKVEIK | 53 |
| S451-1190 | HQ | QVTLRESGPALVKPTQTLTLTCTFS | 1470 |
| | | GFSLTTSGMCVSWIRQPPGKALEWL | |
| | | ARIDWDDDKYYSTSLKARLTISKDT | |
| | | SKNQVVLTMTNMDPVDTATYYCART | |
| | | SVGGTKYYFDYWGQGTLVTVSS | |
| | HC | QVTLRESGPALVKPTQTLTLTCTFS | 1471 |
| | variable | GFSLTTSGMCVSWIRQPPGKALEWL | |
| | | ARIDWDDDKYYSTSLKARLTISKDT | |
| | | SKNQVVLTMTNMDPVDTATYYCAR | |
| | HCDR1 | TSGMCVS | 1472 |
| | HCDR2 | RIDWDDDKYYSTSLKA | 1473 |
| | HCDR3 | TSVGGTKYYFDY | 1474 |
| | HFR1 | QVTLRESGPALVKPTQTLTLTCTFS | 1475 |
| | | GFSLT | |
| | HFR2 | WIRQPPGKALEWLA | 476 |
| | HFR3 | RLTISKDTSKNQVVLTMTNMDPVDT | 517 |
| | | ATYYCAR | |
| | HFR4 | WGQGTLVTVSS | 60 |
| | LC | QSVLTQPPSASGTPGQRVTISCSGS | 1476 |
| | | SSNIGRNTVNWYQQLPGTAPKLLIY | |
| | | SNNQRPSGVPDRFSGSKSGTSASLA | |
| | | ISGLQSEDEADYYCAAWDDSLNGGV | |
| | | FGGGTKLTVL | |
| | LC | QSVLTQPPSASGTPGQRVTISCSGS | 1477 |
| | variable | SSNIGRNTVNWYQQLPGTAPKLLIY | |
| | | SNNQRPSGVPDRFSGSKSGTSASLA | |
| | | ISGLQSEDEADYYCAAWDDSLNG | |
| | LCDR1 | SGSSSNIGRNTVN | 1478 |
| | LCDR2 | SNNQRPS | 119 |
| | LCDR3 | AAWDDSLNGGV | 1479 |
| | LFR1 | QSVLTQPPSASGTPGQRVTISC | 121 |
| | LFR2 | WYQQLPGTAPKLLIY | 122 |
| | LFR3 | GVPDRFSGSKSGTSASLAISGLQSE | 123 |
| | | DEADYYC | |
| | LFR4 | FGGGTKLTVL | 69 |
| S626-84 | HC | QLQLQESGPGLVKPSEALSLTCTVS | 1480 |
| | | GGSISTSNYYWGWIRQPPGKGLEWI | |
| | | GSIYYRGGTHYNPSLKTRVTISVDT | |
| | | SKNQFSLKLSSVTAADTAVYYCARH | |
| | | TYFYDIVGAAVWEPFDIWGQGTMVT | |
| | | VSS | |
| | HC | QLQLQESGPGLVKPSEALSLTCTVS | 1481 |
| | variable | GGSISTSNYYWGWIRQPPGKGLEWI | |
| | | GSIYYRGGTHYNPSLKTRVTISVDT | |
| | | SKNQFSLKLSSVTAADTAVYYCAR | |
| | HCDR1 | TSNYYWG | 1482 |
| | HCDR2 | SIYYRGGTHYNPSLKT | 1483 |
| | HCDR3 | HTYFYDIVGAAVWEPFDI | 1484 |
| | HFR1 | QLQLQESGPGLVKPSEALSLTCTVS | 1485 |
| | | GGSIS | |
| | HFR2 | WIRQPPGKGLEWIG | 7 |
| | HFR3 | RVTISVDTSKNQFSLKLSSVTAADT | 115 |
| | | AVYYCAR | |
| | HFR4 | WGQGTMVTVSS | 44 |
| | LC | EIVLTQSPGTLSLSPGERATLSCRA | 1486 |
| | | SQSVSSSYLAWYQQKPGQAPRLLIS | |
| | | DASSRATGIPDRFSGSGSGTDFTLT | |
| | | ISRLEPEDFAVYYCQQYGSSPPWTF | |
| | | GQGTKVEIK | |
| | LC | EIVLTQSPGTLSLSPGERATLSCRA | 1487 |
| | variable | SQSVSSSYLAWYQQKPGQAPRLLIS | |
| | | DASSRATGIPDRFSGSGSGTDFTLT | |
| | | ISRLEPEDFAVYYCQQYGSSPP | |
| | LCDR1 | RASQSVSSSYLA | 338 |
| | LCDR2 | DASSRAT | 1143 |
| | LCDR3 | QQYGSSPPWT | 1488 |
| | LFR1 | EIVLTQSPGTLSLSPGERATLSC | 50 |
| | LFR2 | WYQQKPGQAPRLLIS | 1489 |
| | LFR3 | GIPDRFSGSGSGTDFTLTISRLEPE | 196 |
| | | DFAVYYC | |
| | LFR4 | FGQGTKVEIK | 53 |
| S626-161 | HC | QLQLQESGPGLVKPSETLSLTCTVS | 1490 |
| | | GGSISSNNYYWGWIRQPPGKGLEWI | |
| | | GSIYYSGSTYYNPSLKSRVTMSVDT | |
| | | SKNQFSLKLSSVTAADTAVYHCARQ | |
| | | GPNYYDRSGYYYVGPFDIWGQGTMV | |
| | | TVSS | |
| | HC | QLQLQESGPGLVKPSETLSLTCTVS | 1491 |
| | variable | GGSISSNNYYWGWIRQPPGKGLEWI | |
| | | GSIYYSGSTYYNPSLKSRVTMSVDT | |
| | | SKNQFSLKLSSVTAADTAVYHCAR | |
| | HCDR1 | SNNYYWG | 1492 |
| | HCDR2 | SIYYSGSTYYNPSLKS | 243 |
| | HCDR3 | QGPNYYDRSGYYYVGPFDI | 1493 |
| | HFR1 | QLQLQESGPGLVKPSETLSLTCTVS | 245 |
| | | GGSIS | |
| | HFR2 | WIRQPPGKGLEWIG | 7 |
| | HFR3 | RVTMSVDTSKNQFSLKLSSVTAADT | 1494 |
| | | AVYHCAR | |
| | HFR4 | WGQGTMVTVSS | 44 |
| | LC | QSVLTQPPSVSAAPGQKVTISCSGS | 1495 |
| | | SSNIGNNSVSWYQHLPGTAPKLLIY | |
| | | ENNERPSGIPDRFSGSKSGTSATLG | |
| | | ITGLQTGDEADYYCETWDRSLSASF | |
| | | GTGTKVTVL | |
| | LC | QSVLTQPPSVSAAPGQKVTISCSGS | 1496 |
| | variable | SSNIGNNSVSWYQHLPGTAPKLLIY | |
| | | ENNERPSGIPDRFSGSKSGTSATLG | |
| | | ITGLQTGDEADYYCETWDRSLSA | |
| | LCDR1 | SGSSSNIGNNSVS | 1497 |
| | LCDR2 | ENNERPS | 1498 |
| | LCDR3 | ETWDRSLSAS | 1499 |
| | LFR1 | QSVLTQPPSVSAAPGQKVTISC | 1500 |
| | LFR2 | WYQHLPGTAPKLLIY | 1501 |
| | LFR3 | GIPDRFSGSKSGTSATLGITGLQTG | 1502 |
| | | DEADYYC | |
| | LFR4 | FGTGTKVTVL | 18 |
| S626-664 | HC | QVQLVQSGAEVKKPGASVKVSCRVS | 1503 |
| | | GYTFTGYYIHWVRQAPGQGLEWMGW | |
| | | INPNSGGTNYAQKFQGRVTMTRDTS | |
| | | ITTAYMELSRLRSDDTAVYYCARVP | |
| | | MILVVDHWGSYFDYWGQGTLVTVSS | |
| | HC | QVQLVQSGAEVKKPGASVKVSCRVS | 1504 |
| | variable | GYTFTGYYIHWVRQAPGQGLEWMGW | |
| | | INPNSGGTNYAQKFQGRVTMTRDTS | |
| | | ITTAYMELSRLRSDDTAVYYCAR | |
| | HCDR1 | GYYIH | 1505 |
| | HCDR2 | WINPNSGGTNYAQKFQG | 953 |
| | HCDR3 | VPMILVVDHWGSYFDY | 1506 |
| | HFR1 | QVQLVQSGAEVKKPGASVKVSCRVS | 1507 |
| | | GYTFT | |
| | HFR2 | WVRQAPGQGLEWMG | 160 |
| | HFR3 | RVTMTRDTSITTAYMELSRLRSDDT | 1508 |
| | | AVYYCAR | |
| | HFR4 | WGQGTLVTVSS | 60 |
| | LC | QSALTQPASVSGSPGQSITISCTGT | 1509 |
| | | SSDVGGYNYVSWYQQYPGKAPKLMI | |
| | | YDVSKRPSGVSNRFSGSKSGNTASL | |
| | | TISGLQAEDEADYYCCSYAGSSALV | |
| | | FGGGTKLTVL | |
| | LC | QSALTQPASVSGSPGQSITISCTGT | 1510 |
| | variable | SSDVGGYNYVSWYQQYPGKAPKLMI | |
| | | YDVSKRPSGVSNRFSGSKSGNTASL | |
| | | TISGLQAEDEADYYCCSYAGSSA | |
| | LCDR1 | TGTSSDVGGYNYVS | 63 |
| | LCDR2 | DVSKRPS | 592 |
| | LCDR3 | CSYAGSSALV | 1511 |
| | LFR1 | QSALTQPASVSGSPGQSITISC | 66 |
| | LFR2 | WYQQYPGKAPKLMIY | 1512 |
| | LFR3 | GVSNRFSGSKSGNTASLTISGLQAE | 68 |
| | | DEADYYC | |
| | LFR4 | FGGGTKLTVL | 69 |
| S728-209 | HC | QITLKESGPTLVKPTQTLTLTCTLS | 1513 |
| | | GFSLSTSGVSVGWIRQPPGKALEWL | |
| | | AVIFWDDDKRYNPSLKSRLTIAKDT | |
| | | SKSQVVLTMTNLDPVDTGTYYCVSG | |
| | | SSYYYYYYMDVWGKGTTVTVSS | |
| | HC | QITLKESGPTLVKPTQTLTLTCTLS | 1514 |
| | variable | GFSLSTSGVSVGWIRQPPGKALEWL | |
| | | AVIFWDDDKRYNPSLKSRLTIAKDT | |
| | | SKSQVVLTMTNLDPVDTGTYYCV | |
| | HCDR1 | TSGVSVG | 1515 |
| | HCDR2 | VIFWDDDKRYNPSLKS | 1516 |
| | HCDR3 | GSSYYYYYYMDV | 1517 |
| | HFR1 | QITLKESGPTLVKPTQTLTLTCTLS | 1518 |
| | | GFSLS | |
| | HFR2 | WIRQPPGKALEWLA | 476 |
| | HFR3 | RLTIAKDTSKSQVVLTMTNLDPVDT | 1519 |
| | | GTYYCVS | |
| | HFR4 | WGKGTTVTVSS | 670 |
| | LC | DIQMTQSPSSVSASIGDRVTITCRA | 1520 |
| | | SQDISTWLAWYQQKPGRAPNLLIYG | |
| | | ASSLQSGVPSRFSGSGSGTDFTLTI | |
| | | SSLQPEDFATYYCQQATSFPLTFGG | |
| | | GTKVEIK | |
| | LC | DIQMTQSPSSVSASIGDRVTITCRA | 1521 |
| | variable | SQDISTWLAWYQQKPGRAPNLLIYG | |
| | | ASSLQSGVPSRFSGSGSGTDFTLTI | |
| | | SSLQPEDFATYYCQQATSFP | |
| | LCDR1 | RASQDISTWLA | 1522 |
| | LCDR2 | GASSLQS | 1523 |
| | LCDR3 | QQATSFPLT | 1524 |
| | LFR1 | DIQMTQSPSSVSASIGDRVTITC | 1525 |
| | LFR2 | WYQQKPGRAPNLLIY | 1526 |
| | LFR3 | GVPSRFSGSGSGTDFTLTISSLQPE | 253 |
| | | DFATYYC | |
| | LFR4 | FGGGTKVEIK | 85 |
| S728-369 | HC | QVQLQESGPGLVKPSQTLSLTCSVS | 1527 |
| | | GGSISSGGYYWSWIRQHPGKGLEWI | |
| | | GYMYYSGSTYYNPSLKSRVTIFVDT | |
| | | SKNHFSLKLTSVTAADTAVYYCARD | |
| | | SYENYYGSGSLEPNYHHYNMDVWGQ | |
| | | GTTVTVSS | |
| | HC | QVQLQESGPGLVKPSQTLSLTCSVS | 1528 |
| | variable | GGSISSGGYYWSWIRQHPGKGLEWI | |
| | | GYMYYSGSTYYNPSLKSRVTIFVDT | |
| | | SKNHFSLKLTSVTAADTAVYYCAR | |
| | HCDR1 | SGGYYWS | 1235 |
| | HCDR2 | YMYYSGSTYYNPSLKS | 1529 |
| | HCDR3 | DSYENYYGSGSLEPNYHHYNMDV | 1530 |
| | HFR1 | QVQLQESGPGLVKPSQTLSLTCSVS | 1531 |
| | | GGSIS | |
| | HFR2 | WIRQHPGKGLEWIG | 1239 |
| | HFR3 | RVTIFVDTSKNHFSLKLTSVTAADT | 1532 |
| | | AVYYCAR | |
| | HFR4 | WGQGTTVTVSS | 147 |
| | LC | DVQMTQSPSTLSASIGDRVTITCRA | 1533 |
| | | SQSISGWLAWYQQRPGKAPKLLIYR | |
| | | ASSLDFGVPSRFSGNGSGTEFTLTI | |
| | | SSLQPDDFATYYCQQYHTYRTFGQG | |
| | | TKVEVK | |
| | LC | DVQMTQSPSTLSASIGDRVTITCRA | 1534 |
| | variable | SQSISGWLAWYQQRPGKAPKLLIYR | |
| | | ASSLDFGVPSRFSGNGSGTEFTLTI | |
| | | SSLQPDDFATYYCQQYHTY | |
| | LCDR1 | RASQSISGWLA | 1535 |
| | LCDR2 | RASSLDF | 1536 |
| | LCDR3 | QQYHTYRT | 1537 |
| | LFR1 | DVQMTQSPSTLSASIGDRVTITC | 1538 |
| | LFR2 | WYQQRPGKAPKLLIY | 1181 |
| | LFR3 | GVPSRFSGNGSGTEFTLTISSLQPD | 1539 |
| | | DFATYYC | |
| | LFR4 | FGQGTKVEVK | 1540 |
| S728-430 | HC | EVQLVESGGGLIQPGGSLRLSCAAS | 1541 |
| | | GFTVSSNYMSWVRQAPGKGLEWVSV | |
| | | IYSGGSTYYADSVKGRFTISRDNSK | |
| | | NTLYLQMNSLRAEDTAVYYCARTPR | |
| | | GSRRGAFDIWGQGTMVTVSS | |
| | HC | EVQLVESGGGLIQPGGSLRLSCAAS | 1542 |
| | variable | GFTVSSNYMSWVRQAPGKGLEWVSV | |
| | | IYSGGSTYYADSVKGRFTISRDNSK | |
| | | NTLYLQMNSLRAEDTAVYYCAR | |
| | HCDR1 | SNYMS | 358 |
| | HCDR2 | VIYSGGSTYYADSVKG | 392 |
| | HCDR3 | TPRGSRRGAFDI | 1543 |
| | HFR1 | EVQLVESGGGLIQPGGSLRLSCAAS | 1544 |
| | | GFTVS | |
| | HFR2 | WVRQAPGKGLEWVS | 130 |
| | HFR3 | RFTISRDNSKNTLYLQMNSLRAEDT | 146 |
| | | AVYYCAR | |
| | HFR4 | WGQGTMVTVSS | 44 |
| | LC | DIQMTQSPSSLSASVGDRVTITCQA | 1545 |
| | | SQDISDYLNWYQQKPGKAPKLLIYD | |
| | | ASNLETGVPSRFSGSGSGTDFTFTI | |
| | | SSLQPEDIATYYCQQYDNLPPLTFG | |
| | | GGTKVEIK | |
| | LC | DIQMTQSPSSLSASVGDRVTITCQA | 1546 |
| | variable | SQDISDYLNWYQQKPGKAPKLLIYD | |
| | | ASNLETGVPSRFSGSGSGTDFTFTI | |
| | | SSLQPEDIATYYCQQYDNLPP | |
| | LCDR1 | QASQDISDYLN | 1547 |
| | LCDR2 | DASNLET | 770 |
| | LCDR3 | QQYDNLPPLT | 1548 |
| | LFR1 | DIQMTQSPSSLSASVGDRVTITC | 251 |
| | LFR2 | WYQQKPGKAPKLLIY | 252 |
| | LFR3 | GVPSRFSGSGSGTDFTFTISSLQPE | 997 |
| | | DIATYYC | |
| | LFR4 | FGGGTKVEIK | 85 |
| S728-537 | HC | QVQLVQSGAEVKKPGASVKVSCKTS | 1549 |
| | | GYTFTGFYLHWLRQAPGQGLEWMGR | |
| | | INPNTGDTDYAQKFQGRVTMTRDTS | |
| | | ISTAYMELSRLRADDTAVYYCARTP | |
| | | GQTRQLFVGTNVLDVWGQGTMVTVS | |
| | | S | |
| | HC | QVQLVQSGAEVKKPGASVKVSCKTS | 1550 |
| | variable | GYTFTGFYLHWLRQAPGQGLEWMGR | |
| | | INPNTGDTDYAQKFQGRVTMTRDTS | |
| | | ISTAYMELSRLRADDTAVYYCAR | |
| | HCDR1 | GFYLH | 1551 |
| | HCDR2 | RINPNTGDTDYAQKFQG | 1552 |
| | HCDR3 | TPGQTRQLFVGTNVLDV | 1553 |
| | HFR1 | QVQLVQSGAEVKKPGASVKVSCKTS | 1381 |
| | | GYTFT | |
| | HFR2 | WLRQAPGQGLEWMG | 1554 |
| | HFR3 | RVTMTRDTSISTAYMELSRLRADDT | 1555 |
| | | AVYYCAR | |
| | HFR4 | WGQGTMVTVSS | 44 |
| | LC | DIQMTQSPSSVSASVGDRVTITCRA | 1556 |
| | | SQGISSWLAWYQQKPGKAPKVLIFA | |
| | | ASSLQSGVPSRFSGSGSGTDFTLTI | |
| | | TSLQPEDFATYFCQQTNSFPPTFGG | |
| | | GTKVEIK | |
| | LC | DIQMTQSPSSVSASVGDRVTITCRA | 1557 |
| | variable | SQGISSWLAWYQQKPGKAPKVLIFA | |
| | | ASSLQSGVPSRFSGSGSGTDFTLTI | |
| | | TSLQPEDFATYFCQQTNSFPP | |
| | LCDR1 | RASQGISSWLA | 1558 |
| | LCDR2 | AASSLQS | 249 |
| | LCDR3 | QQTNSFPPT | 1559 |
| | LFR1 | DIQMTQSPSSVSASVGDRVTITC | 1560 |
| | LFR2 | WYQQKPGKAPKVLIF | 1561 |
| | LFR3 | GVPSRFSGSGSGTDFTLTITSLQPE | 1562 |
| | | DFATYFC | |
| | LFR4 | FGGGTKVEIK | 85 |
| S728-1157 | HC | EVQLVESGGGLVQPGGSLRLSCAAS | 1563 |
| | | GLLVSRNYMNWVRQAPGKGLEWVSI | |
| | | IYSGGSTFYADSVEGRFTISRDESK | |
| | | NTLYLQMNSLRTDDTAVYYCARDLS | |
| | | DYGGIDCWGQGTLVTVSS | |
| | HC | EVQLVESGGGLVQPGGSLRLSCAAS | 1564 |
| | variable | GLLVSRNYMNWVRQAPGKGLEWVSI | |
| | | IYSGGSTFYADSVEGRFTISRDESK | |
| | | NTLYLQMNSLRTDDTAVYYCAR | |
| | HCDR1 | RNYMN | 1565 |
| | HCDR2 | IIYSGGSTFYADSVEG | 1566 |
| | HCDR3 | DLSDYGGIDC | 1567 |
| | HFR1 | EVQLVESGGGLVQPGGSLRLSCAAS | 1568 |
| | | GLLVS | |
| | HFR2 | WVRQAPGKGLEWVS | 130 |
| | HFR3 | RFTISRDESKNTLYLQMNSLRTDDT | 1569 |
| | | AVYYCAR | |
| | HFR4 | WGQGTLVTVSS | 60 |
| | LC | YELTQPLSVSMALGQTARISCGGDN | 1570 |
| | | VGSQNVHWYQQRPGQAPVLVIYRDS | |
| | | NRPSGIPERFSGSKSGNTATLTISR | |
| | | AQAGDEADYYCQVWDSSTVAFGGGT | |
| | | KLTVL | |
| | LC | YELTQPLSVSMALGQTARISCGGDN | 1571 |
| | variable | VGSQNVHWYQQRPGQAPVLVIYRDS | |
| | | NRPSGIPERFSGSKSGNTATLTISR | |
| | | AQAGDEADYYCQVWDSST | |
| | LCDR1 | GGDNVGSQNVH | 1572 |
| | LCDR2 | RDSNRPS | 1573 |
| | LCDR3 | QVWDSSTVA | 1574 |
| | LFR1 | YELTQPLSVSMALGQTARISC | 1575 |
| | LFR2 | WYQQRPGQAPVLVIY | 950 |
| | LFR3 | GIPERFSGSKSGNTATLTISRAQAG | 1576 |
| | | DEADYYC | |
| | LFR4 | FGGGTKLTVL | 69 |
| S728-1261 | HC | QVQLQESGPGLVKPSGTLSLTCAVS | 1577 |
| | | GGSISNNNWWIWVRQPPGKGLEWIG | |
| | | EIHHSGSTDYNPSLKSRVTISIDKS | |
| | | KNQFSLRLSSVTAADTAVYYCARKP | |
| | | EPYYYYYYMDVWGKGTTVTVSS | |
| | HC | QVQLQESGPGLVKPSGTLSLTCAVS | 1578 |
| | variable | GGSISNNNWWIWVRQPPGKGLEWIG | |
| | | EIHHSGSTDYNPSLKSRVTISIDKS | |
| | | KNQFSLRLSSVTAADTAVYYCAR | |
| | HCDR1 | NNNWWI | 1579 |
| | HCDR2 | EIHHSGSTDYNPSLKS | 1580 |
| | HCDR3 | KPEPYYYYYYMDV | 1581 |
| | HFR1 | QVQLQESGPGLVKPSGTLSLTCAVS | 217 |
| | | GGSIS | |
| | HFR2 | WVRQPPGKGLEWIG | 218 |
| | HFR3 | RVTISIDKSKNQFSLRLSSVTAADT | 1582 |
| | | AVYYCAR | |
| | HFR4 | WGKGTTVTVSS | 670 |
| | LC | ETVLTQSPGTLSLSPGERATLSCRA | 1583 |
| | | SQSVSSSYITWYQQKPGQAPRLLIY | |
| | | GASSRATGIPDRFSGSGSGTDFTLT | |
| | | ISRLEPEDFAVYYCQQYRSPWGLTF | |
| | | GPGTKVDIK | |
| | LC | ETVLTQSPGTLSLSPGERATLSCRA | 1584 |
| | variable | SQSVSSSYITWYQQKPGQAPRLLIY | |
| | | GASSRATGIPDRFSGSGSGTDFTLT | |
| | | ISRLEPEDFAVYYCQQYRS | |
| | LCDR1 | RASQSVSSSYIT | 1585 |
| | LCDR2 | GASSRAT | 135 |
| | LCDR3 | QQYRSPWGLT | 1586 |
| | LFR1 | ETVLTQSPGTLSLSPGERATLSC | 1587 |
| | LFR2 | WYQQKPGQAPRLLIY | 182 |
| | LFR3 | GIPDRFSGSGSGTDFTLTISRLEPE | 196 |
| | | DFAVYYC | |
| | LFR4 | FGPGTKVDIK | 443 |
| S728-1690 | HC | QVQLVQSGAEVVKPGSSVKVSCKAS | 1588 |
| | | GGTFTRYAISWVRQAPGQGPEWMGR | |
| | | IIPMFGIANYAQRFQGRVTMTADKS | |
| | | TSTAYMELSSLRSEDTAVYYCATCQ | |
| | | YYYDSSGYGSLDYWGQGTQVTVSS | |
| | HC | QVQLVQSGAEVVKPGSSVKVSCKAS | 1589 |
| | variable | GGTFTRYAISWVRQAPGQGPEWMGR | |
| | | IIPMFGIANYAQRFQGRVTMTADKS | |
| | | TSTAYMELSSLRSEDTAVYYCA | |
| | HCDR1 | RYAIS | 1590 |
| | HCDR2 | RIIPMFGIANYAQRFQG | 1591 |
| | HCDR3 | CQYYYDSSGYGSLDY | 1592 |
| | HFR1 | QVQLVQSGAEVVKPGSSVKVSCKAS | 1593 |
| | | GGTFT | |
| | HFR2 | WVRQAPGQGPEWMG | 1594 |
| | HFR3 | RVTMTADKSTSTAYMELSSLRSEDT | 1595 |
| | | AVYYCAT | |
| | HFR4 | WGQGTQVTVSS | 1596 |
| | LC | EIVLTQSPGTLSLSPGERVTLSCRA | 1597 |
| | | SQSISSNFLAWYQQKPGQAPRLLIS | |
| | | GASSRATGIPDRFSGGGSGTDFTLT | |
| | | ISRLEPEDFAVYYCQQYHSSPRTFG | |
| | | QGTKVEIK | |
| | LC | EIVLTQSPGTLSLSPGERVTLSCRA | 1598 |
| | variable | SQSISSNFLAWYQQKPGQAPRLLIS | |
| | | GASSRATGIPDRFSGGGSGTDFTLT | |
| | | ISRLEPEDFAVYYCQQYHSSP | |
| | LCDR1 | RASQSISSNFLA | 1599 |
| | LCDR2 | GASSRAT | 135 |
| | LCDR3 | QQYHSSPRT | 1600 |
| | LFR1 | EIVLTQSPGTLSLSPGERVTLSC | 1601 |
| | LFR2 | WYQQKPGQAPRLLIS | 1489 |
| | LFR3 | GIPDRFSGGGSGTDFTLTISRLEPE | 1602 |
| | | DFAVYYC | |
| | LFR4 | FGQGTKVEIK | 53 |

TABLE 2
Nucleic Acid Sequences

| | | | SEQ |
|---|---|---|---|
| | | | ID |
| Clone | Description | Sequence | NO |
| S20- | HC-DNA | CAGGTGCAGCTGCAGGAGTCGGGCCCAGGACTGGTGAGGCCTTCGGA | 1603 |
| 15 | | GACCCTGTCCCTCACCTGCACTGTCTCTGGTGGCTCCATCAGTAGTCA | |
| | | CTACTGGAGCTGGATCCGGCAGCCCCCCGGGAAGGGACTGGAGTGGA | |
| | | TTGGGTATATCTATTATAGTGGGAGCACCAATTACAACCCCTCCCTCA | |
| | | AGAGTCGAGTCACCATATCAGTAGACACGTCCAAGAACCAGTTCTCC | |
| | | CTGAAACTTATCTCTGTGACCGCTGCGGACACGGCCGTGTATTACTGT | |
| | | GCGAGAGCCGGGGGCGTTTTTGGAGTGGTTCTGGACTTTGACCACTG | |
| | | GGGCCGGGGAACCCTGGTCACCGTCTCCTCAGCCTCCACCAAGGGCC | |
| | | CATCGGTCTTCCCCCTGGCACCCTCCTCCAAGAGCACCTCTGGGGGCA | |
| | | CAGCGGCCCTGGGCTGCCTGGTCAAGGACTACTTCCCCGAACCGGTG | |
| | | ACGGTGTCGTGGAACTCAGGCGCCCTGACCAGCGGCGTGCACACCTT | |
| | | CCCGGCTGTCCTACAGTCCTCAGGA | |
| | LC-DNA | TCCTATGTGCTGACTCAGCCACCCTCGGTGTCAGTGGCCCCAGGACAG | 1604 |
| | | ACGGCCAGGATTACCTGTGGGGGAAACAACATTGGAAGTAAAAGTGT | |
| | | GCACTGGTACCAGCAGAAGCCAGGCCAGGCCCCTGTGCTGGTCGTCT | |
| | | ATGATGATAGCGACCGGCCCTCAGGGATCCCTGAGCGATTCTCTGGCT | |
| | | CCAACTCTGGGAACACGGCCACCCTGACCATCAGCAGGGTCGAAGCC | |
| | | GGGGATGAGGCCGACTATTACTGTCAGGTGTGGGATAGTAGTAGTGA | |
| | | GCATTATGTCTTCGGAACTGGGACCAAGGTCACCGTCCTAGGTCAGCC | |
| | | CAAGGCCAACCCCACTGTCACTCTGTTCCCGCCCTCCTCTGAGGAGCT | |
| | | CCAAGCCAACAAGGCCACACTAGTGTGTCTGATCAGTGACTTCTACCC | |
| | | GGGAGCTGTGACAGTGGCCTGGAAGGCAGATGGCAGCCCCGTCAAGG | |
| | | CGGGAGTGGAGACCACCAAACCCTCCAAACAGAGCAACAACAAGTA | |
| | | CGCGGCCAGCAGCTA | |
| S20- | HC-DNA | CAGGTGCAGCTGCAGGAGTCGGGCCCAGGACTGGTGAAGCCTTCGGA | 1605 |
| 22 | | GACCCTGTCCCTCACCTGCACTGTCTCTGGTGGCTCCATCAGTAGTTT | |
| | | CTACTGGGGCTGGATCCGGCAGCCCGCCGGGAAGGGACTGGAGTGGA | |
| | | TTGGGCGTTTCCATACTAGTGGGAGCACCAACTACAACCCCTCCTTCA | |
| | | AGAGTCGAGTCACCATGTCAGTAGACACGTCCAAGAACCAGTTCTCC | |
| | | CTGAAGCTGACCTCTGTGACCGCCGCGGACACGGCCGTGTATTACTGT | |
| | | GCGAGCGGCCGGGGCAGCAGCTGGTACGTAGGCTGGTTCTTCGATCT | |
| | | CTGGGGCCGTGGCACCCTGGTCACTGTCTCCTCAGCCTCCACCAAGGG | |
| | | CCCATCGGTCTTCCCCCTGGCACCCTCCTCCAAGAGCACCTCTGGGGG | |
| | | CACAGCAGCCCTGGGCTGCCTGGTCAAGGACTACTTCCCCGAACCGG | |
| | | TGACGGTGTCGTGGAACTCAGGCGCCCTGACCAGCGGCGTGCACACC | |
| | | TTCCCGGCTGTCCTACAGTCCTCAGGA | |
| | LC-DNA | GACATCGTGATGACCCAGTCTCCAGACTCCCTGGCTGTGTCTCTGGGC | 1606 |
| | | GAGAGGGCCACCATCAACTGCAAGTCCAGCCAGACTGTTTTATACAG | |
| | | CTCCAACAATAAGAACTACTTAGCTTGGTACCAGCAGAAACCAGGAC | |
| | | AGCCTCCTAAGTTGCTCATTTACTGGGCATCTACCCGGGAATCCGGGG | |
| | | TCCCTGACCGATTCAGTGGCAGCGGGTCTGGGACAGATTTCACTCTCA | |
| | | CCATCAGCAGCCTGCAGGCTGGAGATGTGGCAGTTTATTACTGTCAGC | |
| | | AATATTATAATACTCCGGACACTTTCGGCGGAGGGACCAAGGTGGAG | |
| | | ATCAATCGAACTGTGGCTGCACCATCTGTCTTCATCTTCCCGCCATCT | |
| | | GATGAGCAGTTGAAATCTGGAACTGCCTCTGTTGTGTGCCTGCTGAAT | |
| | | AACTTCTATCCCAGAGAGGCCAAAGTACAGTGGAAGGTGGATAACGC | |
| S20- | HC-DNA | CAGGTCCAACTCATACAGTCAGGGGCTGAGGTGAAGAAGCCTGGGGC | 1607 |
| 31 | | CTCAGTGAAGGTCTCCTGCACGGCCTCCGGATACTCCCTCAATGAGTT | |
| | | GCCCATACAGTGGGTGCGGCAGGCTCCTGGTAAAGGGCTTGAGTGGA | |
| | | TGGGAGAATTTGATCCCGAAGATGGTGAAACAATCTACGCAGAGAAA | |
| | | TTCCAGGGCAGAGTCACCCTGACCGAGGAAACATCTACAAACACAGC | |
| | | CTACATGGAGTTGAGCAGCCTGAAATCTGAGGACACGGCCGCGTATT | |
| | | TTTGTTCAACCGGCTCGACTATTGGCGTCGTCATTTATGCTTTTGCTAT | |
| | | CTGGGGCCAAGGGACAATGGTCACCGTCTCTTCAGCTTCCACCAAGG | |
| | | GCCCATCGGTCTTCCCCCTGGCGCCCTGCTCCAGGAGCACCTCCGAGA | |
| | | GCACAGCCGCCCTGGGCTGCCTGGTCAAGGACTACTTCCCCGAACCG | |
| | | GTGACGGTGTCGTGGAACTCAGGCGCCCTGACCAGCGGCGTGCACAC | |
| | | CTTCCCGGCTGTCCTACAGTCCTCAGGA | |
| | LC-DNA | GAAATTGTGTTGACGCAGTCCCCAGGCACCCTGTCTTTGTCTCCAGGG | 1608 |
| | | GAAAGAGCCACCCTCTCCTGCAGGGCCAGTCAGGATATTACCAACAA | |
| | | CTTCTTAGCCTGGTACCAGCAGAAAGCCGGCCAGGCTCCCAAACTCTT | |
| | | CATCTATGGTGCATCCAGGAGGGCCCCTGGCATCCCACACAGGTTCA | |
| | | GTGGCAGTGGGTCTGGGACAGACTTCACTCTCACCATCAGCAGCCTG | |
| | | GAGCCTGAAGATTTTGCAGTATATTACTGTCAGCAGTACGGTCCCTCT | |
| | | CCGACGTTCGGCCAAGGGACCAAGGTGGAAATCAAACGAACTGTGGC | |
| | | TGCACCATCTGTCTTCATCTTCCCGCCATCTGATGAGCAGTTGAAATC | |
| | | TGGAACTGCCTCTGTTGTGTGCCTGCTGAATAACTTCTATCCCAGAGA | |
| | | GGCCAAAGTACAGTGGAAGGTGGATAACGCCCTCCAATCGGGTAACT | |
| | | CCCAGGAGAGTGTCACAGAGCAGGACAGCAAGGACAGCACCTACAG | |
| | | CCTCAGCAGCACCCTGACGCTGAGCAAAGCAGACTACGAGAA | |
| S20- | HC-DNA | CAGGTGCAGCTGCAGGAGTCGGGCCCAGGACTGGTGAAGCCTTCGGA | 1609 |
| 40 | | GACCCTGTCCCTCACCTGCACTGTCTCTGGTGGCTCCATCAGTAGTTA | |
| | | CTACTGGAGCTGGATCCGGCAGCCCGCCGGGAAGGGACTGGAGTGGA | |
| | | TTGGGCGTATCTATACCAGTGGGAGCACCAACTACAACCCCTCCCTCA | |
| | | AGAGTCGAGTCACCATGTCAGTAGACACGTCCAAGAACCAGTTCTCC | |
| | | CTGAAGCTGAGCTCTGTGACCGCCGCGGACACGGCCGTGTATTACTGT | |
| | | GCGAGAGGGGGCAGTGGCTGGCGCTTTGACTACTGGGGCCAGGGAAC | |
| | | CCTGGTCACCGTCTCCTCAGGGAGTGCATCCGCCCCAACCCTTTTCCC | |
| | | CCTCGTCTCCTGTGAGAATTCCCCGTCGGATACGAGCAGCGTG | |
| | LC-DNA | CAGTCTGCCCTGACTCAGCCTGCCTCCGTGTCTGGGTCTCCTGGACAG | 1610 |
| | | TCGATCACCATCTCCTGCACTGGAACCAGCAGTGACGTTGGTGGTTAT | |
| | | AACTATGTCTCCTGGTACCAACAGCACCCAGGCAAAGCCCCCAAACT | |
| | | CATGATTTATGATGTCAGTAATCGGCCCTCAGGGGTTTCTAATCGCTT | |
| | | CTCTGGCTCCAAGTCTGGCAACACGGCCTCCCTGACCATCTCTGGGCT | |
| | | CCAGGCTGAGGACGAGGCTGATTATTACTGCAGCTCATATACAAGCA | |
| | | GCAGCACTCTCGGAGTGTTCGGCGGAGGGACCAAGCTGACCGTCCTA | |
| | | GGTCAGCCCAAGGCTGCCCCCTCGGTCACTCTGTTCCCACCCTCCTCT | |
| | | GAGGAGCTTCAAGCCAACAAGGCCACACTGGTGTGTCTCATAAGTGA | |
| | | CTTCTACCCGGGAGCCGTGACAGTGGCCTGGAAGGCAGATAGCAGCC | |
| | | CCGTCAAGGCGGGAGTGGAGACCACCACACCCTCCAAACAAAGCAAC | |
| | | AACAAGTACGCGGCCAGCAGCTA | |
| S20- | HC-DNA | CAGGTGCAGCTGCAGGAGTCGGGCCCAGGACTGGTGAAGCCTTCACA | 1611 |
| 58 | | GACCCTGTCCCTCACCTGCACTGTCTCTGGTGGCTCCATCAACAGTGG | |
| | | TGATTACTACTGGAGCTGGATCCGCCAGCCCCCAGGGAAGGGCCTGG | |
| | | AGTGGATTGGGTACATCTATTTCAGTGGGAGCACCTACTACAACCCGT | |
| | | CCCTCAAGAGTCGAGTTACCATATCACTAGACAGGTCCAAGAACCAG | |
| | | TTCTCCCTGAAGCTGAGCTCTGTGACTGCCGCAGACACGGCCGTGTAT | |
| | | TACTGTGCCAGAGAGGAAAGTATGATTACGCTTGGGGGAGTTATCGT | |
| | | CGACTGGGGCCAGGGAACCCTGGTCACCGTCTCCTCAGCCTCCACCA | |
| | | AGGGCCCATCGGTCTTCCCCCTGGCACCCTCCTCCAAGAGCACCTCTG | |
| | | GGGGCACAGCAGCCCTGGGCTGCCTGGTCAAGGACTACTTCCCCGAA | |
| | | CCGGTGACGGTGTCGTGGAACTCAGGCGCCCTGACCAGCGGCGTGCA | |
| | | CACCTTCCCGGCTGTCCTACAGTCCTCAGGA | |
| | LC-DNA | GATATTGTGATGACCCAGACTCCACTCTCCTCACCTGTCACCCTTGGA | 1612 |
| | | CAGCCGGCCTCCATCTCCTGCAGGTCTAGTCAAAGCCTCGTACACAGT | |
| | | GATGGAGACACCTACTTGAGTTGGCTTCAGCAGAGGCCAGGCCAGCC | |
| | | TCCAAGACTCCTAATTTACAAGATTTCTAACCGGTTCTCTGGGGTCCC | |
| | | AGACAGATTCAGTGGCAGTGGGGCAGGGACAGATTTCACACTGAAAA | |
| | | TCAGCAGGGTGGAAGCTGAGGATGTCGGGGTTTATTACTGCATGCAA | |
| | | GCTACACAATTTCCTCTCACTTTCGGCGGAGGGACCAAGGTGGAGAT | |
| | | CAAACGAACTGTGGCTGCACCATCTGTCTTCATCTTCCCGCCATCTGA | |
| | | TGAGCAGTTGAAATCTGGAACTGCCTCTGTTGTGTGCCTGCTGAATAA | |
| | | CTTCTATCCCAGAGAGGCCAAAGTACAGTGGAAGGTGGATAACGCCC | |
| | | TCCAATCGGGTAACTCCCAGGAGAGTGTCACAGAGCAGGACAGCAAG | |
| | | GACAGCACCTACAGCCTCAGCAGCACCCTGACGCTGAGCAAAGCAGA | |
| | | CTACGAGAA | |
| S20- | HC-DNA | CAGGTGCAGCTGCAGGAGTCGGGCCCAGGACTGGTGAAGCCTTCGGA | 1613 |
| 74 | | GACCCTGTCCCTCACCTGCACTGTCTCTGGTGGCTCCATCAGTAGTCA | |
| | | CTACTGGAGCTGGATCCGGCAGCCCCCAGGGAAGGGACTGGAGCAGA | |
| | | TTGGGTATATGTATTACAGTGGGAGCACCAACTACAACCCCTCCCTCA | |
| | | AGAGTCGAGTCATCATATCAGTAGACACGTCCAAGAACCAGTTCTCC | |
| | | CTGAAGTTGAGCTCTGTGACCGCTGCGGACACGGCCGTGTATTACTGT | |
| | | GCGGGTCGTGACCAGCTGTTATACGGGGCCGATGGTTTTGATATCTGG | |
| | | GGCCAAGGGACAATGGTCACCGTCTCTTCAGCCTCCACCAAGGGCCC | |
| | | ATCGGTCTTCCCCCTGGCACCCTCCTCCAAGAGCACCTCTGGGGGCAC | |
| | | AGCGGCCCTGGGCTGCCTGGTCAAGGACTACTTCCCCGAACCGGTGA | |
| | | CGGTGTCGTGGAACTCAGGCGCCCTGACCAGCGGCGTGCACACCTTC | |
| | | CCGGCTGTCCTACAGTCCTCAGGA | |
| | LC-DNA | CAGTCTGCCCTGACTCAGCCTCCCTCCGCGTCCGGGTCTCCTGGACAG | 1614 |
| | | TCAGTCACCATCTCCTGCACTGGAACCAGCAGTGACGTTGGTGGTTAT | |
| | | AACTATGTCTCCTGGTACCAACAGCACCCAGGCAAAGCCCCCAAACT | |
| | | CATGATTTATGAGGTCAGTAAGCGGCCCTCAGGGGTCCCTGATCGCTA | |
| | | CTCTGGCTCCAAGTCTGGCAACACGGCCTCCCTGACCGTCTCTGGGCT | |
| | | CCAGGCTGAGGATGAGGCTGATTATTACTGCAGCTCATATGCAGGCA | |
| | | GCAGCAATCATGTGATATTCGGCGGAGGGACCAAGCTGACCGTCCTA | |
| | | GGTCAGCCCAAGGCTGCCCCCTCGGTCACTCTGTTCCCGCCCTCCTCT | |
| | | GAGGAGCTTCAAGCCAACAAGGCCACACTGGTGTGTCTCATAAGTGA | |
| | | CTTCTACCCGGGAGCCGTGACAGTGGCCTGGAAGGCAGATAGCAGCC | |
| | | CCGTCAAGGCGGGAGTGGAGACCACCACACCCTCCAAACAAAGCAAC | |
| | | AACAAGTACGCGGCCAGCAGCTA | |
| S20- | HC-DNA | GAAGTGCAGCTGGTGGAGTCTGGGGGAGGCTTGGTACAGCCTGGCAG | 1615 |
| 86 | | GTCCCTGAGACTCTCCTGTGCAGCCTCTGGATTCACCTTTGGTGACTA | |
| | | TGCCATGTACTGGGTCCGGCAACCTCCAGGGAAGGGCCTGGAGTGGG | |
| | | TCTCAGGTATTAGTTGGAATAGAGGTACTATAGGCTATGCGGACTCTG | |
| | | TGAAGGGCCGATTCACCATCTCCAGAGACAACGCCAAGAACTCCCTG | |
| | | TATCTGCAAATGAACAGTCTGACACCTGAGGACACGGCCTTGTATTAC | |
| | | TGTGCAAAAGATATGCTACCAGCTAGTAGGTTCTTCTACTACATGGAC | |
| | | GTCTGGGGCAAAGGGACCACGGTCATCGTCTCCTCAGCCTCCACCAA | |
| | | GGGCCCATCGGTCTTCCCCCTGGCACCCTCCTCCAAGAGCACCTCTGG | |
| | | GGGCACAGCAGCCCTGGGCTGCCTGGTCAAGGACTACTTCCCCGAAC | |
| | | CGGTGACGGTGTCGTGGAACTCAGGCGCCCTGACCAGCGGCGTGCAC | |
| | | ACCTTCCCGGCTGTCCTACAGTCCTCAGGA | |
| | LC-DNA | CAGTCTGCCCTGACTCAGCCTGCCTCCGTGTCTGGGTCTCCTGGACAG | 1616 |
| | | TCGATCACCATCTCCTGCACTGGAACCAGCAGTGACGTTGGTGGTTAT | |
| | | AACTATGTCTCCTGGTACCAACAACACCCAGGCAAAGCCCCCAAACT | |
| | | CATGATTTATGATGTCAGTAATCGGCCCTCAGGGGTTTCTAATCGCTT | |
| | | CTCTGGCTCCAAGTCTGGCAACACGGCCTCCCTGACCATCTCTGGGCT | |
| | | CCAGGCTGAGGACGAGGCTGATTATTACTGCAGCTCATATACAAGCA | |
| | | GCAGCACTCTCGGCGTCTTCGGAACTGGGACCAAGGTCACCGTCCTA | |
| | | GGTCAGCCCAAGGCCAACCCCACTGTCACTCTGTTCCCGCCCTCCTCT | |
| | | GAGGAGCTCCAAGCCAACAAGGCCACACTAGTGTGTCTGATCAGTGA | |
| | | CTTCTACCCGGGAGCTGTGACAGTGGCCTGGAAGGCAGATGGCAGCC | |
| | | CCGTCAAGGCGGGAGTGGAGACCACCAAACCCTCCAAACAGAGCAA | |
| | | CAACAAGTACGCGGCCAGCAGCTA | |
| S24- | HC-DNA | CAGGTGCAGCTGCAGGAGTCGGGCCCAGGACTGGTGAAGCCTTCGGA | 1617 |
| 68 | | GACCCTGTCCCTCACCTGCACTGTCTCTGGTGGCTCCATCACTAGTTA | |
| | | CTACTGGAGCTGGATCCGGCAGCCCCCAGGGAAGGGACTGGAGTGGA | |
| | | TTGAATATATCCATTACAGTGGGAGCACCAACTACAACCCCTCCCTCA | |
| | | AGAGTCGAGTCACCATATCAGTAGACACGTCCAAGAACCAGTTCTCC | |
| | | CTGAAGCTGAGCTCTGTGACCGCTGCGGACACGGCCGTGTATTACTGT | |
| | | GCGAGATTGCTCAAGTATAGCAGGGGGGGGTGCTACTTTGACCACTG | |
| | | GGGCCAGGGAACCCTGGTCACCGTCTCCTCAGCCTCCACCAAGGGCC | |
| | | CATCGGTCTTCCCCCTGGCACCCTCCTCCAAGAGCACCTCTGGGGGCA | |
| | | CAGCGGCCCTGGGCTGCCTGGTCAAGGACTACTTCCCCGAACCGGTG | |
| | | ACGGTGTCGTGGAACTCAGGCGCCCTGACCAGCGGCGTGCACACCTT | |
| | | CCCGGCTGTCCTACAGTCCTCAGGA | |
| | LC-DNA | CAGTCTGTGCTGACTCAGCCACCCTCAGCGTCTGGGACCCCCGGGCA | 1618 |
| | | GAGGGTCACCATCTCTTGTTCTGGAAGCAGCTCCAACATCGGAGGTA | |
| | | ATCCTGTAAACTGGTACCAGCAGCTCCCAGGAACGGCCCCCAAACTC | |
| | | CTCATCTATAGTAATAATCAGCGGCCCTCAGGGGTCCCTGACCGATTC | |
| | | TCTGGCTCCAAGTCTGGCACCTCAGCCTCCCTGGCCATCAGTGGGCTC | |
| | | CAGTCTGAGGATGAGGCTGATTATTACTGTGCAGCATGGGATGACAG | |
| | | CCTGAAGGGTCCGGTATTCGGCGGAGGGACCAAGCTGACCGTCCTAG | |
| | | GTCAGCCCAAGGCTGCCCCCTCGGTCACTCTGTTCCCGCCCTCCTCTG | |
| | | AGGAGCTTCAAGCCAACAAGGCCACACTGGTGTGTCTCATAAGTGAC | |
| | | TTCTACCCGGGAGCCGTGACAGTGGCCTGGAAGGCAGATAGCAGCCC | |
| | | CGTCAAGGCGGGAGTGGAGACCACCACACCCTCCAAACAAAGCAAC | |
| | | AACAAGTACGCGGCCAGCAGCTACCTGAGCCTGACGCCTGAGCAGTG | |
| | | GAAGTCCCACA | |
| S24- | HC-DNA | GAGGTGCAGCTGGTGGAGTCTGGGGGAGGCTTGGTACAGCCGGGGGG | 1619 |
| 105 | | GTCCCTGAGACTCTCCTGTGCAGCCTCTGGATTCACCCTCAGCAGCTA | |
| | | TAGCATGAACTGGGTCCGCCAGGCTCCAGGGAAGGGGCTGGAGTGGG | |
| | | TTTCATACATTAGTAGTAGTAGTAGCACCATATACTACGCAGACTCTG | |
| | | TGAAGGGCCGATTCACCATCTCCAAAGACAACGCCAAGAACTCACTG | |
| | | TATCTGCAAATGAACAGCCTGAGAGCCGAGGACACGGCTGTCTATTA | |
| | | CTGTGCGGTCGGACGGGGATACTTTGTCTACTGGGGCCAGGGAACCC | |
| | | TGGTCACCGTCTCCTCAGCCTCCACCAAGGGCCCATCGGTCTTCCCCC | |
| | | TGGCACCCTCCTCCAAGAGCACCTCTGGGGGCACAGCGGCCCTGGGC | |
| | | TGCCTGGTCAAGGACTACTTCCCCGAACCGGTGACGGTGTCGTGGAA | |
| | | CTCAGGCGCCCTGACCAGCGGCGTGCACACCTTCCCGGCTGTCCTACA | |
| | | GTCCTCAGGA | |
| | LC-DNA | GAAATTGTGTTGACGCAGTCTCCAGGCACCCTGTCTTTGTCTCCAGGG | 1620 |
| | | GAAAGAGCCACCCTCTCCTGCAGGGCCAGTCAGAGTGTTAGCAGCGG | |
| | | TTACTTAGCCTGGTACCAGCAAAAACCTGGCCAGGCTCCCAGGCTCCT | |
| | | CATCTTTGGTGCATCCAGCAGGGCCACTGGCATCCCAGACAGGTTCA | |
| | | GTGGCAGTGGGTCTGGGACAGACTTCACTCTCACCATCAACAGACTG | |
| | | GAGCCTGAAGATTTTGCAGTGTATTACTGTCAGCAGTATGGTAGCTCA | |
| | | CGGACGTTCGGCCAAGGGACCAAGGTGGAAATCAAACGAACTGTGGC | |
| | | TGCACCATCTGTCTTCATCTTCCCGCCATCTGATGAGCAGTTGAAATC | |
| | | TGGAACTGCCTCTGTTGTGTGCCTGCTGAATAACTTCTATCCCAGAGA | |
| | | GGCCAAAGTACAGTGGAAGGTGGATAACGCCCTCCAATCGGGTAACT | |
| | | CCCAGGAGAGTGTCACAGAGCAGGACAGCAAGGACAGCACCTACAG | |
| | | CCTCAGCAGCACCCTGACGCTGAGCAAAGCAGACTACGAGA | |
| S24- | HC-DNA | CAGGTGCAGCTGGTGGAGTCTGGGGGAGGCGTGGTCCAGCCTGGGAG | 1621 |
| 178 | | GTCCCTGAGACTCTCCTGTGCAGCCTCTGGATTCACCTTCAGTAGCTA | |
| | | TGGCATGCACTGGGTCCGCCAGGCTCCAGGCAAGGGGCTGGAGTGGG | |
| | | TGGCAGTTATATGGTATGATGGAAGTAATAAATATTATGCAGACTCC | |
| | | GTGAAGGGCCGATTCACCATCTCCAGAGACAATTCCAAGAACACGCT | |
| | | GTATCTGCAAATGAACAGCCTGAGAGCCGAGGACACGGCTGTGTATT | |
| | | ACTGTGCGAGAATCGAGGGATACAGCTATGGCGACGTGAGGGTCTAC | |
| | | TACTACTACGGTATGGACGTCTGGGGCCAAGGGACCACGGTCACCGT | |
| | | CTCCTCAGCCTCCACCAAGGGCCCATCGGTCTTCCCCCTGGCACCCTC | |
| | | CTCCAAGAGCACCTCTGGGGGCACAGCGGCCCTGGGCTGCCTGGTCA | |
| | | AGGACTACTTCCCCGAACCGGTGACGGTGTCGTGGAACTCAGGCGCC | |
| | | CTGACCAGCGGCGTGCACACCTTCCCGGCTGTCCTACAGTCCTCAGGA | |
| | LC-DNA | CAGTCTGCCCTGACTCAGCCTGCCTCCGTGTCTGGGTCTCCTGGACAG | 1622 |
| | | TCGATCACCATCTCCTGCACTGGAACCACCAGTGACGTTGGTGGTTAT | |
| | | GACTATGTCTCCTGGTACCAACAGCACCCAGGCAAAGCCCCCAAACT | |
| | | CATACTTTCTGAGGTCAGTAATCGGCCCTCAGGGGTTTCTAATCGCTT | |
| | | CTCTGGCTCCAAGTCTGGCAACACGGCCTCCCTGACCATCTCTGGGCT | |
| | | CCAGGCTGAGGACGAGGCTGATTATTACTGCAGCTCATATCCAAGCA | |
| | | GCAGCACTCTAGTCTTCGGAACTGGGACCAAGGTCACCGTCTTAGGTC | |
| | | AGCCCAAGGCCAACCCCACTGTCACTCTGTTCCCGCCCTCCTCTGAGG | |
| | | AGCTCCAAGCCAACAAGGCCACACTAGTGTGTCTGATCAGTGACTTCT | |
| | | ACCCGGGAGCTGTGACAGTGGCCTGGAAGGCAGATGGCAGCCCCGTC | |
| | | AAGGCGGGAGTGGAGACCACCACACCCTCCAAACAAAGCAACAACA | |
| | | AGTACGCGGCCAGCAGCTA | |
| S24- | HC-DNA | CAGGTCCACCTGGTGCAGTCTGGGGCTGAGGTGAAGAAGCCTGGGTC | 1623 |
| 188 | | CTCGGTGAAGGTCTCCTGCAAGGCTTCTGGAGGCACCTTCAGCAGCTG | |
| | | TGCTATCAGCTGGGTGCGACAGGCCCCTGGACAAGGGCTTGAGTGGA | |
| | | TGGGAAGGATCATCCCTATCCTTGGTATAGCAAACTACGCACAGAAG | |
| | | TTCCAGGGCAGAGTCACGATTACCGCGGACAAATCCACGAGCACAGC | |
| | | CTACATGGAGCTGAGCAGCCTGAGATCTGAGGACACGGCCGTGTATT | |
| | | ACTGTGCGAGAGGATGGGAGTTTGGTTCGGGGAGTTATTATCGAACT | |
| | | GATTACTACTACTACGCTATGGACGTCTGGGGCCAAGGGACCACGGT | |
| | | CACCGTCTCCTCAGCCTCCACCAAGGGCCCATCGGTCTTCCCCCTGGC | |
| | | GCCCTGCTCCAGGAGCACCTCTGGGGGCACAGCGGCCCTGGGCTGCC | |
| | | TGGTCAAGGACTACTTCCCCGAACCGGTGACGGTGTCGTGGAACTCA | |
| | | GGCGCCCTGACCAGCGGCGTGCACACCTTCCCGGCTGTCCTACAGTCC | |
| | | TCAGGA | |
| | LC-DNA | CAGTCTGCCCTGACTCAGCCTGCCTCCGTGTCTGGGTCTCCTGGACAG | 1624 |
| | | TCGATCACCATCTCCTGCACTGGAACCAGCAGTGACGTTGGTGGTTAT | |
| | | AACTATGTCTCCTGGTACCAACAGCACCCAGGCAAAGCCCCCAAACT | |
| | | CATGATTTATGAGGTCACTAATCGGCCCTCAGGGGTTTCTAATCGCTT | |
| | | CTCTGGCTCCAGGTCTGGCAACACGGCCTCCCTGACCATCTCTGGGCT | |
| | | CCAGGCTGAGGACGAGGCTGATTATTACTGCAGCTCATATACAAGCA | |
| | | GCAGCCTTTATGTCTTCGGAACTGGGACCAAGGTCGCCGTCCTAGGTC | |
| | | AGCCCAAGGCCAACCCCACTGTCACTCTGTTCCCGCCCTCCTCTGAGG | |
| | | AGCTTCAAGCCAACAAGGCCACACTGGTGTGTCTCATAAGTGACTTCT | |
| | | ACCCGGGAGCCGTGACAGTGGCCTGGAAGGCAGATAGCAGCCCCGTC | |
| | | AAGGCGGGAGTGGAGACCACCAAACCCTCCAAACAGAGCAACAACA | |
| | | AGTACGCGGCCAGCAGCTA | |
| S24- | HC-DNA | GAAGTGCAGCTGGTGCAGTCTGGAGCAGAGGTGAAAAAGCCCGGGG | 1625 |
| 202 | | AGTCTCTGAGGATCTCCTGTAAGGGTTCTGGATACAGCTTTAGCAGCT | |
| | | ACTGGATCAGCTGGGTGCGCCAGATGCCCGGGAAAGGCCTGGAGTGG | |
| | | ATGGGGAGGATTGATCCTAGTGACTCTAACACCAACTACAGCCCGTC | |
| | | CTTCCAAGGCCACGTCACCATCTCAGCTGACAAGTCCATCAGCACTGC | |
| | | CTACCTGCAGTGGAGCAGCCTGAAGGCCTCGGACACCGCCATGTATT | |
| | | ACTGTGCGAGACTCTCCGTCCGGGTATGGTTCGGGGAGTTACCCCATT | |
| | | ACGGTATGGACGTCTGGGGCCAAGGGACCACGGTCACCGTCTCCTCA | |
| | | GCCTCCACCAAGGGCCCATCGGTCTTCCCCCTGGCACCCTCCTCCAAG | |
| | | AGCACCTCTGGGGGCACAGCGGCCCTGGGCTGCCTGGTCAAGGACTA | |
| | | CTTCCCCGAACCGGTGACGGTGTCGTGGAACTCAGGCGCCCTGACCA | |
| | | GCGGCGTGCACACCTTCCCGGCTGTCCTACAGTCCTCAGGA | |
| | LC-DNA | GAAATTGTGTTGACACAGTCTCCAGCCACCCTGTCTTTGTCTCCAGGG | 1626 |
| | | GAAAGAGCCACCCTCTCCTGCAGGGCCAGTCAGAGTGTTAGCAGCTA | |
| | | CCTAGCCTGGTACCAACAGAAACCTGGCCAGGCTCCCAGGCTCCTCA | |
| | | TCTATGATGCATCCAACAGGGCCTCTGGCATCCCAGCCAGGTTCAGTG | |
| | | GCAGTGGGTCAGGGACAGACTTCACTCTCACCATCAGCAGCCTAGAG | |
| | | CCTGAAGATTTTGCAGTTTATTACTGTCAGCAACGTCGCAACTGGCCT | |
| | | CTCACTTTCGGCGGAGGGACCAAGGTGGAGACCAAACGAACTGTGGC | |
| | | TGCACCATCTGTCTTCATCTTCCCGCCATCTGATGAGCAGTTGAAATC | |
| | | TGGAACTGCCTCTGTTGTGTGCCTGCTGAATAACTTCTATCCCAGAGA | |
| | | GGCCAAAGTACAGTGGAAGGTGGATAACGC | |
| S24- | HC-DNA | CAGGTGCAGCTGGTGCAGTCTGGGGCTGAGGTGAAGAAGCCTGGGGC | 1627 |
| 278 | | CTCAGTGAAGGTCTCCTGCAAGGCTTCTGGATACACCTTCACCGGCTA | |
| | | CTATATGCACTGGGTGCGACAGGCCCCTGGACAAGGGCTTGAGTGGA | |
| | | TGGGATGGATCAACCCTAACAGTGGTGACACAAACTATGCACAGAAG | |
| | | TTTCAGGGCTGGGTCACCATGACCAGGGACACGTCCCTCAGCACAGC | |
| | | CTACATGGAGCTGAGCAGGCTGAAATCTGACGACACGGCCGTGTATT | |
| | | ACTGTGCGAGAGTAGGGGTTGGTGAATATAGTGGGAGGCACTACTAC | |
| | | TACTACGGTATGGACGTCTGGGGCCAAGGGACCACGGTCACCGTCTC | |
| | | CTCAGCCTCCACCAAGGGCCCATCGGTCTTCCCCCTGGCACCCTCCTC | |
| | | CAAGAGCACCTCTGGGGGCACAGCGGCCCTGGGCTGCCTGGTCAAGG | |
| | | ACTACTTCCCCGAACCGGTGACGGTGTCGTGGAACTCAGGCGCCCTG | |
| | | ACCAGCGGCGTGCACACCTTCCCGGCTGTCCTACAGTCCTCAGGA | |
| | LC-DNA | GAAATTGTGTTGACGCAGTCTCCAGGCACCCTGTCTTTGTCTCCAGGG | 1628 |
| | | GAAAGAGCCACCCTCTCCTGCAGGGCCAGTCAGAGTATTAGCAGCAG | |
| | | CTACTTAGCCTGGTACCAGCAGAAACCTGGCCAGGCTCCCAGGCTCCT | |
| | | CATCTATGGTGCATCCAGCAGGGCCACTGGCATCCCAGACAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTATGGTAGCTCA | |
| | | CTCACTTTCGGCGGAGGGACCAAGGTGGAGATCAAACGAACTGTGGC | |
| | | TGCACCATCTGTCTTCATCTTCCCGCCATCTGATGAGCAGTTGAAATC | |
| | | TGGAACTGCCTCTGTTGTGTGCCTGCTGAATAACTTCTATCCCAGAGA | |
| | | GGCCAAAGTACAGTGGAAGGTGGATAACGC | |
| S24- | HC-DNA | GAGGTGCAGCTGGTGGAGTCTGGGGGAGGCTTGGTACAGCCAGGGCG | 1629 |
| 339 | | GTCCCTGAGACTCTCCTGTACAGCTTCTGGATTCACCTTTGGTGATTAT | |
| | | GCTATGAGCTGGTTCCGCCAGGCTCCAGGGAAGGGGCTGGAGTGGGT | |
| | | AGGTTTCATTAGAAGCAAAGCTTATGGTGGGACAACACAACACGCCG | |
| | | CCTCTGTGAAAGGCAGATTCACCATCTCAAGAGATGATTCCAAAAGC | |
| | | ATCGCCTATCTGCAAATGAACAGCCTGAAAACCGAGGACACAGCCGT | |
| | | GTATCACTGTGCTAGAGATGGATATGATTGTAGTGGTGGTAGATGCTA | |
| | | CTCCCATATATTTGACTACTGGGGCCAGGGAACCCTGGTCACCGTCTC | |
| | | CTCAGGTGAGTCCTCACCACCCCCTCTCTGAGTCCACTTAGGGAGACT | |
| | | CAGCTTGCCAGGGTCTCAGGGTCAGAGTCTTGTAG | |
| | LC-DNA | GAAATAGTGATGACGCAGTCTCCAGCCACCCTGTCTGTGTCTCCAGGG | 1630 |
| | | GAAAGAGCCACCCTCTCCTGCAGGGCCAGTCAGAGTGTTAGCAGCAA | |
| | | CTTAGCCTGGTACCAGCAGAAACCTGGCCAGGCTCCCAGGCTCCTCAT | |
| | | CTATGGTGCATCCACCAGGGCCACTGGTATCCCAGCCAGGTTCAGTG | |
| | | GCAGTGGGTCTGGGACAGAGTTCACTCTCACCATCAGCAGCCTGCAG | |
| | | TCTGAAGATTTTGCAGTTTATTACTGTCAGCAGTATGATAACTGGTGG | |
| | | ACGTTCGGCCAAGGGACCAAGGTGGAAATCAAACGAACTGTGGCTGC | |
| | | ACCATCTGTCTTCATCTTCCCGCCATCTGATGAGCAGTTGAAATCTGG | |
| | | AACTGCCTCTGTTGTGTGCCTGCTGAATAACTTCTATCCCAGAGAGGC | |
| | | CAAAGTACAGTGGAAGGTGGATAACGC | |
| S24- | HC-DNA | CAGGTGCAGCTGCAGGAGTCGGGCCCAGGACTGGTGAAGCCTTCGGG | 1631 |
| 472 | | GACCCTGTCCCTCACCTGCGCTGTCTCTGGTGGCTCCATCAGCAGTAT | |
| | | TAACTGGTGGAGTTGGGTCCGCCAGCCCCCAGGGAAGGGGCTGGAGT | |
| | | GGATCGGGGAAATCTATCATAGTGGGAACACCAACTATAACCCGTCC | |
| | | CTCAAGAGTCGAGTCACCATATCAGGAGACAAGTCCAAGAACCAGTT | |
| | | CTCCCTGAAGCTGAGCTCTGTGACCGCCGCGGACACGGCCGTGTATTA | |
| | | CTGTGCGAGAGGTTACTATGATAGTAGTCCTTATTACGAGCCACAGG | |
| | | GAATTGACTACTGGGGCCAGGGAATCCTGGTCACCGTCTCCTCAGCCT | |
| | | CCACCAAGGGCCCATCGGTCTTCCCCCTGGCACCCTCCTCCAAGAGCA | |
| | | CCTCTGGGGGCACAGCGGCCCTGGGCTGCCTGGTCAAGGACTACTTC | |
| | | CCCGAACCGGTGACGGTGTCGTGGAACTCAGGCGCCCTGACCAGCGG | |
| | | CGTGCACACCTTCCCGGCTGTCCTACAGTCCTCAGGA | |
| | LC-DNA | CAGCTTGTGCTGACTCAATCGCCCTCTGCCTCTGCCTCCCTGGGAGCC | 1632 |
| | | TCGGTCAAGCTCACCTGCACTCTGAGCAGTGGGCACAGCAGCTACAC | |
| | | CATCGCATGGCATCAGCAGCAGCCAGAGAAGGGCCCTCGGTACTTGA | |
| | | TGAAAGTTAACAGTGATGGCAGCCACACCAAGGGGGACGGGATCCCT | |
| | | GATCGCTTCTCAGGCTCCAGCTCTGGGGCTGAGCGCTACCTCACCATC | |
| | | TCCAGCCTCCAGTCTGAGGATGAGGCTGACTATTACTGTCAGACCTGG | |
| | | GGCACTGGCATTCGAGTATTCGGCGGAGGGACCAAGCTGACCGTCCT | |
| | | AGGTCAGCCCAAGGCTGCCCCCTCGGTCACTCTGTTCCCGCCCTCCTC | |
| | | TGAGGAGCTTCAAGCCAACAAGGCCACACTGGTGTGTCTCATAAGTG | |
| | | ACTTCTACCCGGGAGCCGTGACAGTGGCCTGGAAGGCAGATAGCAGC | |
| | | CCCGTCAAGGCGGGAGTGGAGACCACCACACCCTCCAAACAAAGCAA | |
| | | CAACAAGTACGCGGCCAGCAGCTA | |
| S24- | HC-DNA | CAGGTGCAGCTGGTGCAGTCTGGGGCTGAGGTGAAGAAGCCTGGGGC | 1633 |
| 490 | | CTCAGTGAAGGTTTCCTGCAAGGCATCTGGATACACCTTCACCAGCTA | |
| | | CTTTATTCACTGGGTGCGACAGGCCCCTGGACAAGGGCTTGAGTGGA | |
| | | TGGGAATAATCAACCCTAGTGGTGGTAGCACAAGCTACGCACAGAAG | |
| | | TTCCAGGGCAGAGTCACCATGACCAGGGACACGTCCACGAGCACAGT | |
| | | CTACATGGAGCTGAGCAGCCTGAGATCTGAGGACACGGCCGTGTATT | |
| | | ACTGTGCGAGACACACAACCCCGACAAGATACTTTGACTACTGGGGC | |
| | | CAGGGAACCCTGGTCACCGTCTCCTCAGGGAGTGCATCCGCCCCAAC | |
| | | CCTTTTCCCCCTCGTCTCCTGTGAGAATTCCCCGTCGGATACGAGCAG | |
| | | CGTG | |
| | LC-DNA | GAAATTGTGTTGACGCAGTCTCCAGGCACCCTGTCTTTGTCTCCAGGG | 1634 |
| | | GAAAGAGCCACCCTCTCCTGCAGGGCCAGTCAGAGTGTTACCAGCAG | |
| | | CTACTTAGCCTGGTACCAGCAGAGACGTGGCCAGGCTCCCAGGCTCC | |
| | | TCATCTATGGTGCATCCAGCAGGGCCACTGGCATCCCAGACAGGTTC | |
| | | AGTGGCAGTGGGTCTGGGACAGACTTCACTCTCACCATCAGCAGACT | |
| | | GGAGCCTGAAGATTTTGCAGTGTATTACTGTCAGCAGTATGGTAGCTC | |
| | | ACCTCTCACTTTCGGCGGAGGGACCAAGGTGGAGATCAAACGAACTG | |
| | | TGGCTGCACCATCTGTCTTCATCTTCCCGCCATCTGATGAGCAGTTGA | |
| | | AATCTGGAACTGCCTCTGTTGTGTGCCTGCTGAATAACTTCTATCCCA | |
| | | GAGAGGCCAAAGTACAGTGGAAGGTGGATAACGC | |
| S24- | HC-DNA | CAGCTGCAGCTGCAGGAGTCGGGCCCAGGACTGGTGAAGCCTTCGGA | 1635 |
| 494 | | GACCCTGTCCCTCACCTGCACTGTCTCTGGTGGCTCCATCAGCAGTAG | |
| | | TAGTTACTACTGGGGCTGGATCCGCCAGCCCCCAGGGAAGGGGCTGG | |
| | | AGTGGATTGGGAGTATCTATTATAGTGGGAGCACCTACTACAACCCG | |
| | | TCCCTCAAGAGTCGAGTCACCATATCCGTAGACACGTCCAAGAACCA | |
| | | GTTCTCCCTGAAGCTGAGCTCTGTGACCGCCGCAGACACGGCTGTGTA | |
| | | TTACTGTGCGAGAAAGCCACGTAGTGACTACGGGTACTTCGATCTCTG | |
| | | GGGCCGTGGCACCCTGGTCACTGTCTCCTCAGCCTCCACCAAGGGCCC | |
| | | ATCGGTC | |
| | LC-DNA | GACATCCAGATGACCCAGTCTCCATCCTCCCTGTCTGCATCTGTAGGA | 1636 |
| | | GACAGAGTCACCATCACTTGCCGGGCAAGTCAGAGCATTAGCAGCTA | |
| | | TTTAAATTGGTATCAGCAGAAACCAGGGAAAGCCCCTAAGCTCCTGA | |
| | | TCTATGCTGCATCCAGTTTGCAAAGTGGGGTCCCATCAAGGTTCAGTG | |
| | | GCAGTGGATCTGGGACAGATTTCACTCTCACCATCAGCAGTCTGCAAC | |
| | | CTGAAGATTTTGCAACTTACTACTGTCAACAGAGTTACAGTACCCCTC | |
| | | AACTCACTTTCGGCGGAGGGACCAAGGTGGAGATCAAACGAACTGTG | |
| | | GCTGCACCATCTGTCTTCATCTTCCCGCCATCTGATGAGCAGTTGAAA | |
| | | TCTGGAACTGCCTCTGTTGTGTGCCTGCTGAATAACTTCTATCCCAGA | |
| | | GAGGCCAAAGTACAGTGGAAGGTGGATAACGC | |
| S24- | HC-DNA | GAGGTGCAGCTGGTGGAGTCTGGGGGAGGCTTGGTAAAGCCAGGGCG | 1637 |
| 566 | | GTCCCTGAGACTCTCCTGTACAGCTTCTGGATTCACCTTTGGTGATTAT | |
| | | GCTATGAGCTGGTTCCGCCAGGCTCCAGGGAAGGGGCTGGAGTGGGT | |
| | | AGGTTTCACTAGAAGGAAAGCTTATGGTGGGACAACAGAGTACGCCG | |
| | | CGTCTGTGAAAGGCAGATTCACCATCTCAAGAGATGATTCCAAAAGC | |
| | | ATCGCCTATCTGCAAATGAACAGCCTGAAAACCGAGGACACAGCCGT | |
| | | GTATTACTGTACTAGAATTAAGGTGGGCCGTTTCGATCTTACCGACAG | |
| | | TGGGAGCTACCGATACTTTGACTACTGGGGCCAGGGAACCCTGGTCA | |
| | | CCGTCTCCTCAGCCTCCACCAAGGGCCCATCGGTCTTCCCCCTGGCAC | |
| | | CCTCCTCCAAGAGCACCTCTGGGGGCACAGCGGCCCTGGGCTGCCTG | |
| | | GTCAAGGACTACTTCCCCGAACCGGTGACGGTGTCGTGGAACTCAGG | |
| | | CGCCCTGACCAGCGGCGTGCACACCTTCCCGGCTGTCCTACAGTCCTC | |
| | | AGGA | |
| | LC-DNA | GATATTGTGATGACTCAGTCTCCACTCTCCCTGCCCGTCACCCCTGGA | 1638 |
| | | GAGCCGGCCTCCATCTCCTGCAGGTCTAGTCAGAGCCTCCTGCATAGT | |
| | | AATGGATACAACTATTTGGATTGGTACCTGCAGAAGCCAGGGCAGTC | |
| | | TCCACAGCTCCTGATCTATTTGGGTTCTAATCGGGCCTCCGGGGTCCC | |
| | | TGACAGGTTCAGTGGCAGTGGATCAGGCACAGATTTTACACTGAAAA | |
| | | TCAGCAGAGTGGAGGCTGAGGATGTTGGGGTTTATTACTGCATGCAA | |
| | | CCTCTACAAACTCCTTGGACGTTCGGCCAAGGGACCAAGGTGGAAAT | |
| | | CAAACGAACTGTGGCTGCACCATCTGTCTTCATCTTCCCGCCATCTGA | |
| | | TGAGCAGTTGAAATCTGGAACTGCCTCTGTTGTGTGCCTGCTGAATAA | |
| | | CTTCTATCCCAGAGAGGCCAAAGTACAGTGGAAGGTGGATAACGC | |
| S24- | HC-DNA | GAGGTGCAGCTGGTGGAGTCTGGGGGAGGCTTGGTCCAGCCTGGGGG | 1639 |
| 636 | | GTCCCTGAGACTCTCCTGTGCAGCCTCTGGATTCACCTTAAGTAGCTA | |
| | | TTGGATGAGCTGGGTCCGCCAGGCTCCAGGGAAGGGGCTGGAGTGGG | |
| | | TGGCCAACATAAAGCAAGATGGAAGTGAGAAATACTATGTGGACTCT | |
| | | GTGAAGGGCCGATTCACCATCTCCAGAGACAACGCCAAGAACTCACT | |
| | | GTATCTGCAAATGAACAGCCTGAGAGCCGAGGACACGGCCGTGTATT | |
| | | ACTGTGCGAGAGATCTAACTGCCACCTGGTTCGACCCCTGGGGCCAG | |
| | | GGAACCCTGGTCACCGTCTCCTCAGCACCCACCAAGGCTCCGGATGT | |
| | | GTTCCCCATCATATCAGGGTGCAGACACCCAAAGGATAACAGCCCTG | |
| | | TGGTCCTGGCATGCTTGATAACTGGGTACCACC | |
| | LC-DNA | CAGACTGTGGTGACCCAGGAGCCATCGTTCTCAGTGTCCCCTGGAGG | 1640 |
| | | GACAGTCACACTCACTTGTGGCTTGAGCTCTGGCTCAGTCTCTACTAG | |
| | | TTACTACCCCAGCTGGTACCAGCAGACCCCAGGCCAGGCTCCACGCA | |
| | | CGCTCATCTACAGCACAAACAAACGCTCTTCTGGGGTCCCTGATCGCT | |
| | | TCTCTGGCTCCATCCTTGGGAACAAAGCTGCCCTCACCATCACGGGGG | |
| | | CCCAGGCAGATGATGAATCTGATTATTACTGTGTGCTCTATATGGGTA | |
| | | GTGGCATGTCGGTGTTCGGCGGAGGGACCAAGCTGACCGTCCTAGGT | |
| | | CAGCCCAAGGCTGCCCCCTCGGTCACTCTGTTCCCGCCCTCCTCTGAG | |
| | | GAGCTTCAAGCCAACAAGGCCACACTGGTGTGTCTCATAAGTGACTT | |
| | | CTACCCGGGAGCCGTGACAGTGGCCTGGAAGGCAGATAGCAGCCCCG | |
| | | TCAAGGCGGGAGTGGAGACCACCACACCCTCCAAACAAAGCAACAA | |
| | | CAAGTACGCGGCCAGCAGCTA | |
| S24- | HC-DNA | CAGGTCCAGCTTGTGCAGTCTGGGGCTGAGGTGAAGAAGCCTGGGGC | 1641 |
| 740 | | CTCAGTGAAGGTTTCCTGCAAGGCTTCTGGATACACCTTCACTAGCTA | |
| | | TGCTTTGCATTGGGTGCGCCAGGCCCCCGGACAAAGGCTTGAGTGGA | |
| | | TGGGATGGATCAACGCTGGCAATGGTAACACAAAATATTCACAGAGG | |
| | | TTCCAGGGCAGAGTCACCATTATTAGGGACACATCCGCGAGCACAAC | |
| | | CTACATGGAGCTGAGCAGCCTGAGATCTGAAGACACGGCTGTGTATT | |
| | | ACTGTGCGAGAGGCTATGCCCGAGCCGGGGTTATTACTATCAAAGAA | |
| | | TCACTCCACCACTGGGGCCAGGGCACCCTGGTCACCGTCTCCTCAGCC | |
| | | TCCACCAAGGGCCCATCGGTCTTCCCCCTGGCACCCTCCTCCAAGAGC | |
| | | ACCTCTGGGGGCACAGCGGCCCTGGGCTGCCTGGTCAAGGACTACTT | |
| | | CCCCGAACCGGTGACGGTGTCGTGGAACTCAGGCGCCCTGACCAGCG | |
| | | GCGTGCACACCTTCCCGGCTGTCCTACAGTCCTCAGGA | |
| | LC-DNA | GACATCGTGATGACCCAGTCTCCAGACTCCCTGGCTGTGTCTCTGGGC | 1642 |
| | | GAGAGGGCCACCATCAACTGCAAGTCCAGCCAGAGTGTTTTATACAG | |
| | | CTCCAACAATAAGAACTACTTAGCCTGGTACCAGCAGAAACCAGGAC | |
| | | AGCCTCCTAAGCTGCTCATTTACTGGGCATCTACCCGGGAATCCGGGG | |
| | | TCCCTGACCGATTCAGTGGCAGCGGGTCTGGGACAGATTTCACTCTCA | |
| | | CCATCAGCAGCCTGCAGGCTGAAGATGTGGCAGTTTATTACTGTCAGC | |
| | | AATATTATAGTACTCCTCCCCTCACTTTCGGCGGAGGGACCAAGGTGG | |
| | | AGATCAAACGAACTGTGGCTGCACCATCTGTCTTCATCTTCCCGCCAT | |
| | | CTGATGAGCAGTTGAAATCTGGAACTGCCTCTGTTGTGTGCCTGCTGA | |
| | | ATAACTTCTATCCCAGAGAGGCCAAAGTACAGTGGAAGGTGGATAAC | |
| | | GC | |
| S24- | HC-DNA | CAGGTGCAGCTGCAGGAGTCGGGCCCAGGACTGGTGAAGCCTTCGGA | 1643 |
| 791 | | GACCCTGTCCCTCACCTGCACTGTCTCTGGTGGCTCCATCAGTAGTTC | |
| | | CTACTGGAGCTGGATCCGGCAGCCCCCAGGGAAGGGACTGGAGTGGA | |
| | | TTGGGTATATCTATTACAGTGGGAACACCAACTACAACCCCTCCCTCA | |
| | | AGAGTCGAGTCACCCTATCAATAGACACGTCCAAGAACCAGTTCTCC | |
| | | CTGAAGCTGAGCTCTGTGACCGCTGCGGACACGGCCGTGTATTACTGT | |
| | | GCGTGCAGTGTTACGATTTTTGGAGTGGTTACCCCTGCTTTTGATATCT | |
| | | GGGGCCAAGGGACAATGGTCACCGTCTCTTCAGCCTCCACCAAGGGC | |
| | | CCATCGGTCTTCCCCCTGGCACCCTCCTCCAAGAGCACCTCTGGGGGC | |
| | | ACAGCGGCCCTGGGCTGCCTGGTCAAGGACTACTTCCCCGAACCGGT | |
| | | GACGGTGTCGTGGAACTCAGGCGCCCTGACCAGCGGCGTGCACACCT | |
| | | TCCCGGCTGTCCTACAGTCCTCAGGA | |
| | LC-DNA | GAGATTGTGTTGACGCACTCTCCAGGCACCCTGTCTTTGTCTCCAGGG | 1644 |
| | | GAAAGAGCCACCCTCTCCTGCAGGGCCAGTCAGAGTGTCCGCAGCTA | |
| | | CTTAGCCTGGTACCAGCAGAAACCTGGCCAGGCTCCCAGGCTCCTCAT | |
| | | CTATGGTGCATCCAGCAGGGCCACTGGCATCCCAGACAGGTTCAGTG | |
| | | GCAGTGGGTCTGGGACAGACTTCACTCTCACCATCAGCAGACTGGAG | |
| | | CCTGACGATTTTGCAGTGTATTACTGTCAGCAGTATGGTAGCTCACCT | |
| | | TGGACGTTCGGCCAAGGGACCAAGGTGGAAATCAAACGAACTGTGGC | |
| | | TGCACCATCTGTCTTCATCTTCCCGCCATCTGATGAGCAGTTGAAATC | |
| | | TGGAACTGCCTCTGTTGTGTGCCTGCTGAATAACTTCTATCCCAGAGA | |
| | | GGCCAAAGTACAGTGGAAGGTGGATAACGCCCTCCAATCGGGTAACT | |
| | | CCCAGGAGAGTGTCACAGAGCAGGACAGCAAGGACAGCACCTACAG | |
| | | CCTCAGCAGCACCCTGACGCTGAGCAAAGCAGACTACGAG | |
| S24- | HC-DNA | CAGGTCCAGCTGGTGCAGTCTGGGGCTGAGGTGAAGAAGCCTGGGTC | 1645 |
| 902 | | CTCGGTGAAGGTCTCCTGCAAGGCTTCTGGAGGCACCTTCAGCAGCTA | |
| | | TGCTATCAGCTGGGTGCGACAGGCCCCTGGACAAGGGCTTGAGTGGA | |
| | | TGGGAAGGATCATCCCTATCCTTGGTATAGCAAACTACGCACAGAAG | |
| | | TTCCAGGGCAGAGTCACGATTACCGCGGACAAATCCACGAGCACAGC | |
| | | CTACATGGAGCTGAGCAGCCTGAGATCTGAGGACACGGCCGTGTATT | |
| | | ACTGTGCGAGATGGGATTTTGGAGTGGTTATTCAATACGGTATGGAC | |
| | | GTCTGGGGCCAAGGGACCACGGTCACCGTCTCCTCAGCCTCCACCAA | |
| | | GGGCCCATCGGTCTTCCCCCTGGCACCCTCCTCCAAGAGCACCTCTGG | |
| | | GGGCACAGCGGCCCTGGGCTGCCTGGTCAAGGACTACTTCCCCGAAC | |
| | | CGGTGACGGTGTCGTGGAACTCAGGCGCCCTGACCAGCGGCGTGCAC | |
| | | ACCTTCCCGGCTGTCCTACAGTCCTCAGGACTCTACTCCCTCAGCAGC | |
| | | GTGGTGACCGTGCCCTCCAGCAGCTTGGG | |
| | LC-DNA | CAGGCTGTGGTGACTCAGGAGCCCTCACTGACTGTGTCCCCAGGAGG | 1646 |
| | | GACAGTCACTCTCACCTGTGGCTCCAGCACTGGAGCTGTCACCAGTGG | |
| | | TCATTATCCCTACTGGTTCCAGCAGAAGCCTGGCCAAGCCCCCAGGAC | |
| | | ACTGATTTATGATACAAGCAACAAACACTCCTGGACACCTGCCCGGTT | |
| | | CTCAGGCTCCCTCCTTGGGGGCAAAGCTGCCCTGACCCTTTCGGGTGC | |
| | | GCAGCCTGAGGATGAGGCTGAGTATTACTGCTTGCTCTCCTATAGTGG | |
| | | TTGGGTGTTCGGCGGAGGGACCAAGCTGACCGTCCTAGGTCAGCCCA | |
| | | AGGCTGCCCCCTCGGTCACTCTGTTCCCGCCCTCCTCTGAGGAGCTTC | |
| | | AAGCCAACAAGGCCACACTGGTGTGTCTCATAAGTGACTTCTACCCG | |
| | | GGAGCCGTGACAGTGGCCTGGAAGGCAGATAGCAGCCCCGTCAAGGC | |
| | | GGGAGTGGAGACCACCACACCCTCCAAACAAAGCAACAACAAGTAC | |
| | | GCGGCCAGCAGCTA | |
| S24- | HC-DNA | CAGGTGCAGCTGCAGGAGTCGGGCCCAGGACTGGTGAAGCCTTCGGA | 1647 |
| 921 | | GACCCTGTCCCTCACCTGCACTGTCTCTGGTGGCTCCATCAATAGTTT | |
| | | CTACTGGAACTGGATCCGGCAGCCCCCCGGGAAGGGACTGGAGTGGA | |
| | | TTGGGTATATCTATTACAGTGGGAACACCAAGTACAACCCCTCCCTCA | |
| | | AGAGTCGAGTCACCATATCAGTAGACACGTCCAACAGCCAGTTCTCC | |
| | | CTGAAGCTGAGCTCTGTGACCGCTGCGGACACGGCCGTGTATTACTGT | |
| | | GCGGCGCTCAAAAAGCAGGAGCTGGTATCGTTGCAGGCTTTTGATAT | |
| | | CTGGGGCCAAGGGACAATGGTCACCGTCTCTTCAGCCTCCACCAAGG | |
| | | GCCCATCGGTCTTCCCCCTGGCACCCTCCTCCAAGAGCACCTCTGGGG | |
| | | GCACAGCGGCCCTGGGCTGCCTGGTCAAGGACTACTTCCCCGAACCG | |
| | | GTGACGGTGTCGTGGAACTCAGGCGCCCTGACCAGCGGCGTGCACAC | |
| | | CTTCCCGGCTGTCCTACAGTCCTCAGGA | |
| | LC-DNA | GACATCCAGATGACCCAGTCTCCATCCTCCCTGTCTGCATCTCTGGGA | 1648 |
| | | GACGGGGTCACCATCACTTGCCGGGCAAGTCAGAGCATTAGCAGCTA | |
| | | TTTAAGTTGGTATCAGCAGAAACCCGGGAAAGCCCCTAAGCTCCTGA | |
| | | TCTATGCTGCATCCAGTTTGCAAAGTGGGGTCCCATCAAGGTTCAGTG | |
| | | GCAGTGGATCTGGGACAGATTTCACTCTCACCATCAGCAGTCTGCAAC | |
| | | CTGAAGATTTTGCAACTTACTACTGTCAACAGAGTTACAATACCCCCG | |
| | | TGACGTTCGGCCAAGGGACCAAGGTGGAAATCAAACGAACTGTGGCT | |
| | | GCACCATCTGTCTTCATCTTCCCGCCATCTGATGAGCAGTTGAAATCT | |
| | | GGAACTGCCTCTGTTGTGTGCCTGCTGAATAACTTCTATCCCAGAGAG | |
| | | GCCAAAGTACAGTGGAAGGTGGATAACGCAGATCGGAAGAGC | |
| S24- | HC-DNA | CAGGTGCAGCTGCAGGAGTCGGGCCCAGGACTGGTGAAGCCTTCGGA | 1649 |
| 1063 | | GACCCTGTCCCTCACCTGCACTGTCTCTGGTGGCTCCATCAGTAGTTA | |
| | | CTACTGGAGCTGGATCCGGCAGCCCCCAGGGAAGGGACTGGAGTGGA | |
| | | TTGGATATATCTATTACAGTGGGAGCACCAAGTACAACCCCTCCCTCA | |
| | | AGAGTCGAGTCACCATATCAGTAGACACGTCCAAGAACCAGTTCTCC | |
| | | CTGAAGCTGACCTCTGTGACCGCTGCGGACACGGCCGTGTATTACTGT | |
| | | GCGAGAATCTATGATAGTAGTGGTTATTACCATCCCGTCTTTGACTAC | |
| | | TGGGGCCAGGGAACCCTGGTCACCGTCTCCTCAGCCTCCACCAAGGG | |
| | | CCCATCGGTCTTCCCCCTGGCACCCTCCTCCAAGAGCACCTCTGGGGG | |
| | | CACAGCGGCCCTGGGCTGCCTGGTCAAGGACTACTTCCCCGAACCGG | |
| | | TGACGGTGTCGTGGAACTCAGGCGCCCTGACCAGCGGCGTGCACACC | |
| | | TTCCCGGCTGTCCTACAGTCCTCAGGA | |
| | LC-DNA | GAAATTGTGTTGACGCAGTCTCCAGGCACCCTGTCTTTGTCTCCAGGG | 1650 |
| | | GAAAGAGCCACCCTCTCCTGCAGGGCCAGTCAGAGTGTTAGCAGCAG | |
| | | CTACTTAGCCTGGTACCAGCAGAAACCTGGCCAGGCTCCCAGGCTCCT | |
| | | CATCTATGGTGCATCCAGCAGGGCCACTGACATCCCAGACAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTATGGTAGCTCA | |
| | | CCGTGGACGTTCGGCCAAGGGACCAAGGTGGAAATCAAACGAACTGT | |
| | | GGCTGCACCATCTGTCTTCATCTTCCCGCCATCTGATGAGCAGTTGAA | |
| | | ATCTGGAACTGCCTCTGTTGTGTGCCTGCTGAATAACTTCTATCCCAG | |
| | | AGAGGCCAAAGTACAGTGGAAGGTGGATAACGC | |
| S24- | HC-DNA | CAGGTGCAGCTGGTGCAGTCTGGGGCTGAGGTGAAGAAGCCTGGGGC | 1651 |
| 1224 | | CTCAGTGAGGGTTTCCTGCAAGGCATCTGGATACACCTTCACCAGCTA | |
| | | CTATATCTACTGGGTGCGACAGGCCCCTGGACAAGGGCTTGAGTGGA | |
| | | TGGGAGTAATCAACCCTAGTGGTGGTAGCACAAGCTACGCACAGAAG | |
| | | TTCCAGGGCAGAGTCACCTTGACCAGGGACACGTCCACGAGCACAGT | |
| | | CTACATGGACCTGAGCAGTCTGAGATCTGAGGACACGGCCGTGTATT | |
| | | ACTGTGCGAGAGATCCTATAATGTGGGAGGTAGTAACTCGGGGGAGG | |
| | | GGCAACTGGTTCGACCCCTGGGGCCAGGGAACCCTGGTCACCGTCTC | |
| | | CTCAGCCTCCACCAAGGGCCCATCGGTCTTCCCCCTGGCACCCTCCTC | |
| | | CAAGAGCACCTCTGGGGGCACAGCGGCCCTGGGCTGCCTGGTCAAGG | |
| | | ACTACTTCCCCGAACCGGTGACGGTGTCGTGGAACTCAGGCGCCCTG | |
| | | ACCAGCGGCGTGCACACCTTCCCGGCTGTCCTACAGTCCTCAGGA | |
| | LC-DNA | CAGTCTGTGCTGACGCAGCCGCCCTCAGTGTCTGGGGCCCCAGGGCA | 1652 |
| | | GAGGGTCACCATCCCCTGCACTGGGAGCAGCTTCAACATCGGGGCAG | |
| | | GTTATGATGTACACTGGTACCAGCAGCTTCCAGGAACAGCCCCCAAA | |
| | | CTCCTCATCTTTGGTAACAGCAATCGGCCCTCAGGGGTCCCTGACCGA | |
| | | TTCTCTGGCTCCAGGTCTGGCACCTCAGCCTCCCTGGCCATCACTGGG | |
| | | CTCCAGGCTGAGGATGAGGCTGATTATTACTGCCAGTCCTATGACAGT | |
| | | AGCCTGAGTGGTGTGGTATTCGGCGGAGGGACTACGCTGACCGTCCT | |
| | | AGGTCAGCCCAAGGCTGCCCCCTCGGTCACTCTGTTCCCGCCCTCCTC | |
| | | TGAGGAGCTTCAAGCCAACAAGGCCACACTGGTGTGTCTCATAAGTG | |
| | | ACTTCTACCCGGGAGCCGTGACAGTGGCCTGGAAGGCAGATAGCAGC | |
| | | CCCGTCAAGGCGGGAGTGGAGACCACCACACCCTCCAAACAAAGCAA | |
| | | CAACAAGTACGCGGCCAGCAGCTACCTGAGCCTGACGCCTGAGCAGT | |
| | | GGAAGTCCCAC | |
| S24- | HC-DNA | GAGGTGCAGCTGGTGGAGTCTGGGGGAGGCTTGGTCCAGCCTGGGGG | 1653 |
| 1271 | | GTCCCTGAGACTCTCCTGTGCAGCCTCTGGATTCACCGTCAGTAGCAA | |
| | | CTACATGAGCTGGGTCCGCCAGGCTCCAGGGAAGGGGCTGGAGTGGG | |
| | | TCTCAGTTATTTATAGCGATGGTAACACATACTATGCAGACTCCGTGA | |
| | | AGGGCAGATTCACCATCTCCAGAGACAATTCCAAGAACATGTTATAT | |
| | | CTTCAAATGAACAGCCTGAGAGCCGAGGACACGGCTGTGTATTACTG | |
| | | TGCGAGAGACCCCGGCCAGGGGTATTGTAGTGGTGGTAGCTGCGCTC | |
| | | CGTCCTATTCTCTTGACTACTGGGGCCAGGGAACCCTGGTCACTGTCT | |
| | | CCTCAGGGAGTGCATCCGCCCCAACCCTTTTCCCCCTCGTCTCCTGTG | |
| | | AGAATTCCCCGTCGGATACGAGCAGCGTG | |
| | LC-DNA | TCCTATGAGCTGACTCAGCCACCCTCAGTGTCCGTGTCCCCAGGACAG | 1654 |
| | | ACAGCCAGCATCACCTGCTCTGGGGATAAATTGGGGGATAGATATGT | |
| | | TTGTTGGTATCAGCAGAAGCCAGGTCAGTCCCCTGTGCTGGTCATCTA | |
| | | TCAAGATACCAAGCGGCCCTCAGGGATCCCTGAGCGATTCTCTGGCTC | |
| | | CAACTCTGGGAACACAGCCACTCTGACCATCAGCGGGACCCAGGCTA | |
| | | TGGATGAGGCTGACTATTACTGTCAGGCGTGGGACAGCAGCACTTGG | |
| | | GTGTTCGGCGGAGGGACCAAGCTGACCGTCCTGGGTCAGCCCAAGGC | |
| | | TGCCCCCTCGGTCACTCTGTTCCCGCCCTCCTCTGAGGAGCTTCAAGC | |
| | | CAACAAGGCCACACTGGTGTGTCTCATAAGTGACTTCTACCCGGGAG | |
| | | CCGTGACAGTGGCCTGGAAGGCAGATAGCAGCCCCGTCAAGGCGGGA | |
| | | GTGGAGACCACCACACCCTCCAAACAAAGCAACAACAAGTACGCGGC | |
| | | CAGCAGCTA | |
| S24- | HC-DNA | GAGGTGCAGCTGGTGGAGTCTGGAGGAGGCTTGGTCCAGCCTGGGGG | 1655 |
| 1339 | | GTCCCTGAGACTCTCCTGTGCAGCCTCTGGGTTCACCGTCAGTAGCAA | |
| | | CTACATGAGCTGGGTCCGCCAGGCTCCAGGGAAGGGGCTGGAGTGGG | |
| | | TCTCAGATATTTATAGCGGTGGTAGCACATACTACGCAGACTCCGTGA | |
| | | AGGGCCGATTCACCATCTCCAGACACAATTCCAAGAACACGCTGTAT | |
| | | CTTCAAATGAACAGCCTGAGAGCTGAGGACACGGCCGTGTATTACTG | |
| | | TGCGAGAGATCGACGGGGATACAGCTATGGTTTGCACCACGGTATGG | |
| | | ACGTCTGGGGCCAAGGGACCACGGTCACCGTCTCCTCAGCCTCCACC | |
| | | AAGGGCCCATCGGTCTTCCCCCTGGCACCCTCCTCCAAGAGCACCTCT | |
| | | GGGGGCACAGCGGCCCTGGGCTGCCTGGTCAAGGACTACTTCCCCGA | |
| | | ACCGGTGACGGTGTCGTGGAACTCAGGCGCCCTGACCAGCGGCGTGC | |
| | | ACACCTTCCCGGCTGTCCTACAGTCCTCAGGA | |
| | LC-DNA | GAAATTGTGTTGACGCAGTCTCCAGGCACCCTGTCTTTGTCTCCAGGG | 1656 |
| | | GAAAGAGCCACCCTCTCCTGCAGGGCCAGTCAGAGTGTTAGCAGCAG | |
| | | CTACTTAGCCTGGTACCAGCAGAAACCTGACCAGGCTCCCAGGCTCCT | |
| | | CATCTATGGTGCATCCAGCAGGGCCACTGGCATCCCAGACAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTATGGTAGCTCA | |
| | | CCTAACACTTTTGGCCAGGGGACCAAGCTGGAGATCAAACGAACTGT | |
| | | GGCTGCACCATCTGTCTTCATCTTCCCGCCATCTGATGAGCAGTTGAA | |
| | | ATCTGGAACTGCCTCTGTTGTGTGCCTGCTGAATAACTTCTATCCCAG | |
| | | AGAGGCCAAAGTACAGTGGAAGGTGGATAACGC | |
| S24- | HC-DNA | CAGCTGCAGCTGCAGGAGTCGGGCCCAGGACTGGTGAAGCCTTCGGA | 1657 |
| 1345 | | GACCCTGTCCCTCACCTGCACTGTCTCTGGTGGCTCCATCAGCAGTAG | |
| | | TAGTTACTACTGGGGCTGGATCCGCCAGCCCCCAGGGAAGGGGCTGG | |
| | | AGTGGATTGGGAGTATCTATTATAGTGGGAGCACCTACTACAACCCG | |
| | | TCCCTCAAGAGTCGAGTCACCATATCCGTAGACACGTCCAAGAACCA | |
| | | GTTCTCCCTGAAGCTGAGCTCTGTGACCGCCGCAGACACGGCTGTGTA | |
| | | TTACTGTGCGAGACGAATCAGACGCCCCACCTCGGAAGTGGTTATTA | |
| | | CTTATGTCTTTGACTACTGGGGCCAGGGAACCCTGGTCACCGTCTCCT | |
| | | CAGCACCCACCAAGGCTCCGGATGTGTTCCCCATCATATCAGGGTGC | |
| | | AGACACCCAAAGGATAACAGCCCTGTGGTCCTGGCATGCTTGATAAC | |
| | | TGGGTACCACC | |
| | LC-DNA | GCCATCCAGTTGACCCAGTCTCCATCCTCCCTGTCTGCATCTGTAGGA | 1658 |
| | | GACAGAGTCACCATCACTTGCCGGGCAAGTCAGGGCATTAGCAGTGC | |
| | | TTTAGCCTGGTATCAGCAGAAACCAGGGAAAGCTCCTAAGCTCCTGA | |
| | | TCTATGATGCCTCCAGTTTGGAAAGTGGGGTCCCATCAAGGTTCAGCG | |
| | | GCAGTGGATCTGGGACAGATTTCACTCTCACCATCAGCAGCCTGCAG | |
| | | CCTGAAGATTTTGCAACTTATTACTGTCAACAGTTTAATAGTTACCTC | |
| | | ACTTTCGGCGGAGGGACCAAGGTGGAGATCAAACGAACTGTGGCTGC | |
| | | ACCATCTGTCTTCATCTTCCCGCCATCTGATGAGCAGTTGAAATCTGG | |
| | | AACTGCCTCTGTTGTGTGCCTGCTGAATAACTTCTATCCCAGAGAGGC | |
| | | CAAAGTACAGTGGAAGGTGGATAACGCCCTCCAATCGGGTAACTCCC | |
| | | AGGAGAGTGTCACAGAGCAGGACAGCAAGGACAGCACCTACAGCCT | |
| | | CAGC | |
| S24- | HC-DNA | GAGGTGCAGCTGGTGGAGTCTGGAGGAGGCTTGGTCCAGCCTGGGGG | 1659 |
| 1378 | | GTCCCTGAGACTCTCCTGTGCAGCCTCTGGGTTCACCGTCAGTAGCAA | |
| | | CTACATGAGCTGGGTCCGCCAGGCTCCAGGGAAGGGGCTGGAGTGGG | |
| | | TCTCAGTTATTTATAGCGGTGGTAGCACATACTACGCAGACTCCGTGA | |
| | | AGGGCCGATTCACCATCTCCAGACACAATTCCAAGAACACGCTGTAT | |
| | | CTTCAAATGAACAGCCTGAGAGCTGAGGACACGGCCGTGTATTACTG | |
| | | TGCGAGAGAAGGATATTGTACTAATGGTGTATGCTATAGGCATGCTTT | |
| | | TGATATCTGGGGCCAAGGGACAATGGTCACCGTCTCTTCAGGGAGTG | |
| | | CATCCGCCCCAACCCTTTTCCCCCTCGTCTCCTGTGAGAATTCCCCGTC | |
| | | GGATACGAGCAGCGTG | |
| | LC-DNA | CAGACTGTGGTGACCCAGGAGCCATCGTTCTCAGTGTCCCCTGGAGG | 1660 |
| | | GACAGTCACACTCACTTGTGGCTTGAGCTCTGGCTCAGTCTCTACTAG | |
| | | TTACTACCCCAGCTGGTACCAGCAGACCCCAGGCCAGGCTCCACGCA | |
| | | CGCTCATCTACAGCACAAACACTCGCTCTTCTGGGGTCCCTGATCGCT | |
| | | TCTCTGGCTCCATCCTTGGGAACAAAGCTGCCCTCACCATCACGGGGG | |
| | | CCCAGGCAGATGATGAATCTGATTATTACTGTGTGCTGTATATGGGTA | |
| | | GTGGCATTTCGGTGTTCGGCGGAGGGACCAAGCTGACCGTCCTAGGT | |
| | | CAGCCCAAGGCTGCCCCCTCGGTCACTCTGTTCCCGCCCTCCTCTGAG | |
| | | GAGCTTCAAGCCAACAAGGCCACACTGGTGTGTCTCATAAGTGACTT | |
| | | CTACCCGGGAGCCGTGACAGTGGCCTGGAAGGCAGATAGCAGCCCCG | |
| | | TCAAGGCGGGAGTGGAGACCACCACACCCTCCAAACAAAGCAACAA | |
| | | CAAGTACGCGGCCAGCAGCTA | |
| S24- | HC-DNA | CAGGTGCAGCTGCAGGAGTCGGGCCCAGGACTGGTGAAGCCTTCGGA | 1661 |
| 1379 | | GACCCTGTCCCTCACCTGCACTGTCTCTGGTGGCTCCATCAGTAGTTA | |
| | | CTACTGGAGCTGGATCCGGCAGCCCCCAGGGAAGGGACTGGAGTGGA | |
| | | TTGGGTATATCTATTACAGTGGGAGCACCAACTACAACCCCTCCCTCA | |
| | | AGAGTCGAGTCACCATATCAGTAGACACGTCCAAGAACCAGTTCTCC | |
| | | CTGAAGCTGAGCTCTGTGACCGCTGCGGACACGGCCGTGTATTACTGT | |
| | | GCGAGAGATTACTATCAACTCCCTATGGACGTCTGGGGCCAAGGGAC | |
| | | CACGGTCACCGTCTCCTCAGCCTCCACCAAGGGCCCATCGGTCTTCCC | |
| | | CCTGGCACCCTCCTCCAAGAGCACCTCTGGGGGCACAGCGGCCCTGG | |
| | | GCTGCCTGGTCAAGGACTACTTCCCCGAACCGGTGACGGTGTCGTGG | |
| | | AACTCAGGCGCCCTGACCAGCGGCGTGCACACCTTCCCGGCTGTCCTA | |
| | | CAGTCCTCAGGA | |
| | LC-DNA | CAGTCTGTGCTGACTCAGCCACCCTCAGCGTCTGGGACCCCCGGGCA | 1662 |
| | | GAGGGTCACCATCTCTTGTTCTGGAAGCAGCTCCAACATCGGAAGTA | |
| | | ATTATGTATACTGGTACCAGCAGCTCCCAGGAACGGCCCCCAAACTC | |
| | | CTCATCTATAGGAATAATCAGCGGCCCTCAGGGGTCCCTGACCGATTC | |
| | | TCTGGCTCCAAGTCTGGCACCTCAGCCTCCCTGGCCATCAGTGGGCTC | |
| | | CGGTCCGAGGATGAGGCTGATTATTACTGTGCAGCATGGGATGACAG | |
| | | CCTGAGTGGTCGGGTGTTCGGCGGAGGGACCAAGCTGACCGTCCTAG | |
| | | GTCAGCCCAAGGCTGCCCCCTCGGTCACTCTGTTCCCGCCCTCCTCTG | |
| | | AGGAGCTTCAAGCCAACAAGGCCACACTGGTGTGTCTCATAAGTGAC | |
| | | TTCTACCCGGGAGCCGTGACAGTGGCCTGGAAGGCAGATAGCAGCCC | |
| | | CGTCAAGGCGGGAGTGGAGACCACCACACCCTCCAAACAAAGCAAC | |
| | | AACAAGTACGCGGCCAGCAGCTA | |
| S24- | HC-DNA | GAGGTGCAGCTGGTGGAGTCTGGGGGAGGCTTGGTACAGCCTGGGGG | 1663 |
| 1384 | | GTCCCTGAGACTCTCCTGTGCAGTCTCTGGATTCACCTTCAGTAGCTA | |
| | | TAGCATGAACTGGGTCCGCCAGGCTCCAGGGAAGGGGCTGGAGTGGG | |
| | | TTTCATACATTAGTAGTAGTAGTAGTATCATATACTACGCAGACTCTG | |
| | | TGAAGGGCCGATTCACCATCTCCAGAGACAACGCCAAGAACTCACTG | |
| | | TATCTGCAAATGAACAGCCTGAGAGCCGAGGACACGGCTGTGTATTA | |
| | | CTGTGCGAGAGATTTCCTCGACTATAGCAGGTCGTATTCGTACGGTAT | |
| | | GGACGTCTGGGGCCAAGGGACCACGGTCACCGTCTCCTCAGCCTCCA | |
| | | CCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCC | |
| | | GAACCGGTGACGGTGTCGTGGAACTCAGGCGCCCTGACCAGCGGCGT | |
| | | GCACACCTTCCCGGCTGTCCTACAGTCCTCAGGA | |
| | LC-DNA | TCCTATGTGCTGACTCAGCCACCCTCGGTGTCAGTGGCCCCAGGACAG | 1664 |
| | | ACGGCCAGGATTACCTGTGGGGGAGACAACATTGGAAGTAAAAATGT | |
| | | GCACTGGTACCAGCAGAAGCCCGGCCAGGCCCCTGTGCTGGTCGTCT | |
| | | TTGATGATAGCGACCGGCCCTCAGGGATCCCTGAGCGATTCTCTGGCT | |
| | | CCAACTCTGGGAACACGGCCACCCTGACCATCAGCAGGGTCGAAGCC | |
| | | GGGGATGAGGCCGACTATTACTGTCAGGTGTGGGATAGTAGTAGTGA | |
| | | TCACTATGTGGTATTCGGCGGAGGGACCAAGCTGACCGTCCTAGGTC | |
| | | AGCCCAAGGCTGCCCCCTCGGTCACTCTGTTCCCGCCCTCCTCTGAGG | |
| | | AGCTTCAAGCCAACAAGGCCACACTGGTGTGTCTCATAAGTGACTTCT | |
| | | ACCCGGGAGCCGTGACAGTGGCCTGGAAGGCAGATAGCAGCCCCGTC | |
| | | AAGGCGGGAGTGGAGACCACCACACCCTCCAAACAAAGCAACAACA | |
| | | AGTACGCGGCCAGCAGCTACC | |
| S24- | HC-DNA | GAGGTGCAGCTGGTGGAGTCTGGGGGAGGCTTGGTACAGCCAGGGCG | 1665 |
| 1476 | | GTCCCTGAGACTCTCCTGTACAGCTTCTGGATTCACCTTTGGTGATTAT | |
| | | GCTATGAGCTGGTTCCGCCAGGCTCCAGGGAAGGGGCTGGAGTGGGT | |
| | | AGGTTTCATTAGAAGCAAAGCTTATGGTGGGACAACACAATACGCCG | |
| | | CCTCTGTGAAAGGCAGATTCACCATCTCAAGAGATGATTCCAAAAGC | |
| | | ATCGCCTATCTGCAAATGAACAGCCTGAAAACCGAGGACACAGCCGT | |
| | | GTATTACTGTACTAGAGTACGATATTGTACTAATGGTGTATGCTATGG | |
| | | CTACCACTTTGACTACTGGGGCCAGGGAACCGTGGTCACCGTCTCCTC | |
| | | AGCCTCCACC | |
| | LC-DNA | GAAATAGTGATGACGCAGTCTCCAGCCACCCTGTCTGTGTCTCCAGGG | 1666 |
| | | GAAAGAGCCACCCTCTCCTGCAGGGCCAGTCAGAGTGTTAGCAGCAA | |
| | | CTTAGCCTGGTACCAGCAGAAACCTGGCCAGGCTCCCAGGCTCCTCAT | |
| | | CTATGGTGCATCCACCAGGGCCACTGGTATCCCAGCCAGGTTCAGTG | |
| | | GCAGTGGGTCTGGGACAGAGTTCACTCTCACCATCAGCAGCCTGCAG | |
| | | TCTGAAGATTTTGCAGTTTATTACTGTCAGCAGTATAATAACTGGTGG | |
| | | ACGTTCGGCCAAGGGACCAAGGTGGAAATCAAACGAACTGTGGCTGC | |
| | | ACCATCTGTCTTCATCTTCCCGCCATCTGATGAGCAGTTGAAATCTGG | |
| | | AACTGCCTCTGTTGTGTGCCTGCTGAATAACTTCTATCCCAGAGAGGC | |
| | | CAAAGTACAGTGGAAGGTGGATAACGC | |
| S24- | HC-DNA | CAGGTGCAGCTGCAGGAGTCGGGCCCAGGACTGGTGAAGCCTTCGGA | 1667 |
| 1564 | | GACCCTGTCCCTCACCTGCACTGTCTCTGGTGGCTCCATCAGTAGTTA | |
| | | CTACTGGAGCTGGATCCGTCAGCCCCCAGGGAAGGGGCTGGAGTGGA | |
| | | TTGGCTATGTCTATTACAGTGGGAACACCAAATACAACCCCTCCCTCA | |
| | | AGAGTCGAGTCACCATATCAGTAGACACGTCCAAGAACCAGTTCTCC | |
| | | CTGAAGCTGGGCTCTGTGACCGCCGCGGACACGGCCGTTTATTATTGT | |
| | | GCGAGACATTCGAGGATAGAAGTGGCTGGTACTCTAGACTTTGACTA | |
| | | CTGGGGCCAGGGAACCCTGGTCACCGTCTCCTCAGCCTCCACCAAGG | |
| | | GCCCATCGGTCTTCCCCCTGGCACCCTCCTCCAAGAGCACCTCTGGGG | |
| | | GCACAGCGGCCCTGGGCTGCCTGGTCAAGGACTACTTCCCCGAACCG | |
| | | GTGACGGTGTCGTGGAACTCAGGCGCCCTGACCAGCGGCGTGCACAC | |
| | | CTTCCCGGCTGTCCTACAGTCCTCAGGA | |
| | LC-DNA | GACATCCAGATGACCCAGTCTCCATCCTCCCTGTCTGCATCTGTAGGA | 1668 |
| | | GACCGGGTCACCATCACTTGCCGGGCAAGTCAGAGCATTAGAAGCTA | |
| | | TTTAAATTGGTATCAGCAGAAACGAGGGAAAGCCCCTAAGCTCCTGA | |
| | | TCTATGCTGCATCCAGTTTGCAAAGTGGGGTCCCATCAAGGTTCAGTG | |
| | | GCAGTGGATCTGGGACAGATTTCACTCTCACCATCAGCAGTCTGCAAC | |
| | | CTGAAGATTTTGCAACTTACTACTGTCAACAGAGTTACAGTACCCCTC | |
| | | CGACGTTCGGCCAAGGGACCAAGGTGGAAATCAAACGAACTGTGGCT | |
| | | GCACCATCTGTCTTCATCTTCCCGCCATCTGATGAGCAGTTGAAATCT | |
| | | GGAACTGCCTCTGTTGTGTGCCTGCTGAATAACTTCTATCCCAGAGAG | |
| | | GCCAAAGTACAGTGGAAGGTGGATAACGC | |
| S24- | HC-DNA | CAGGTGCAGCTGGTGGAGTCTGGGGGAGGCGTGGTCCAGCCTGGGAG | 1669 |
| 1636 | | GTCCCTGAGACTCTCCTGTGCAGCCTCTGGATTCACCTTCAGTAACTA | |
| | | TGGCATGCACTGGGTCCGCCAGGCTCCAGGCAAGGGGCTGGAGTGGG | |
| | | TGGCCGTTATATGGTATGATGGAAGTAATAAATACTATGCAGACTCC | |
| | | GTGAAGGGCCGATTCACCATCTCCAGAGACAATTCCAAGAACACGCT | |
| | | GTATCTGCAAATGAACAGCCTGAGAGCCGAGGACACGGCTGTGTATT | |
| | | ACTGTGCGAGAGGAGATTGTACTAATGGTGTATGCCATCCCCTTCTAA | |
| | | TTTATTATGATAGTAGTGGTTTAGACTACTGGGGCCAGGGAACCCTGG | |
| | | TCACCGTCTCCTCAGCCTCCACCAAGGGCCCATCGGTCTTCCCCCTGG | |
| | | CACCCTCCTCCAAGAGCACCTCTGGGGGCACAGCGGCCCTGGGCTGC | |
| | | CTGGTCAAGGACTACTTCCCCGAACCGGTGACGGTGTCGTGGAACTC | |
| | | AGGCGCCCTGACCAGCGGCGTGCACACCTTCCCGGCTGTCCTACAGTC | |
| | | CTCAGGA | |
| | LC-DNA | GAAATTGTGTTGACACAGTCTCCAGCCACCCTGTCTTTGTCTCCAGGG | 1670 |
| | | GAAAGAGCCACCCTCTCCTGCAGGGCCAGTCAGAGTGTTAGCAGCTA | |
| | | CTTAGCCTGGTACCAACAGAAACCTGGCCAGGCTCCCAGGCTCCTCAT | |
| | | CTATGATGCATCCAACAGGGCCACTGGCATCCCAGCCAGGTTCAGTG | |
| | | GCAGTGGGTCTGGGACAGACTTCACTCTCACCATCAGCAGCCTAGAG | |
| | | CCTGAAGATTTTGCAGTTTATTACTGTCAGCAGCGTAGCAACTGGCCT | |
| | | CCGATCACTTTCGGCCCTGGGACCAAAGTGGATATCAAACGAACTGT | |
| | | GGCTGCACCATCTGTCTTCATCTTCCCGCCATCTGATGAGCAGTTGAA | |
| | | ATCTGGAACTGCCTCTGTTGTGTGCCTGCTGAATAACTTCTATCCCAG | |
| | | AGAGGCCAAAGTACAGTGGAAGGTGGATAACGCCCTCCAATCGGGTA | |
| | | ACTCCCAGGAGAGTGTCACAGAGCAGGACAGCAAGGACAGCACCTA | |
| | | CAGCCTC | |
| S24- | HC-DNA | CAGGTGCAGCTGGTGGAGTCTGGGGGAGGCGTGGTCCAGCCTGGGAG | 1671 |
| 1002 | | GTCCCTGAGACTCTCCTGTGCAGCCTCTGGATTCACCTTCACTAGCTA | |
| | | TGCTATGCACTGGGTCCGCCAGGCTCCAGGCAAGGGGCTGGAGTGGG | |
| | | TGGCAGTTATATCATATGATGGAGGCAGTAAATACTACGCAGACTCC | |
| | | GTGAAGGGCCGATTCACCATCTCCAGAGACAATTCCAAGAACACGCT | |
| | | GTATCTGCAAATGAACAGCCTGAGAGCTGAGGACACGGCTGTGTATT | |
| | | ACTGTGCGAGGACTACACCGGGTATAACAGCAGCTGGAACAGGGACC | |
| | | CTAGGGAGATACTACTACTACGGTATGGACGTCTGGGGCCAAGGGAC | |
| | | CACGGTCACCGTCTCCTCAGGGAGTGCATCCGCCCCAACCCTTTTCCC | |
| | | CCTCGTCTCCTGTGAGAATTCCCCGTCGGATACGAGCAGCGTG | |
| | LC-DNA | GCCATCCAGTTGACCCAGTCTCCATCCTCCCTGTCTGCATCTGTAGGA | 1672 |
| | | GACAGAGTCACCATCACTTGCCGGGCAAGTCAGGGCATTAGCAGTGC | |
| | | TTTAGCCTGGTATCAGCAGACACCAGGGAAAGCTCCTAAGCTCCTGA | |
| | | TCTATGATGCCTCCAGTTTGGAAAGTGGGGTCCCGTCAAGGTTCAGCG | |
| | | GCAGTGGATCTGGGACAGATTTCTCTCTCACCATCGGCAGCCTGCAGC | |
| | | CTGAAGATTTTGCAAGTTATTACTGTCAACAGTTTAATAGTTACCCTC | |
| | | TCACTTTCGGCGGAGGGACCAAGGTGGAGATCAAACGAACTGTGGCT | |
| | | GCACCATCTGTCTTCATCTTCCCGCCATCTGATGAGCAGTTGAAATCT | |
| | | GGAACTGCCTCTGTTGTGTGCCTGCTGAATAACTTCTATCCCAGAGAG | |
| | | GCCAAAGTACAGTGGAAGGTGGATAACGCCCTCCAATCGGGTAACTC | |
| | | CCAGGAGAGTGTCACAGAGCAGGACAGCAAGGACAGCACCTACAGC | |
| | | CTCAGCAGCACCCTGACGCTGAGCAAAGCAGACTACGAGA | |
| S24- | HC-DNA | CAGGTCCAACTGGTACAGTCTGGGGCTGAGGTGAAGAAGCCTGGGGC | 1673 |
| 1301 | | CTCAGTGAAGGTCTCCTGCAAGGTTTCCGGATACACCCTCATTGAATT | |
| | | ATCCATGCACTGGGTGCGACAGGCTCCTGGAAAAGGGCTTGAGTGGA | |
| | | TGGGAGGTTTTGATCCTGAAGATGGTGAAACAATCTACGCACAGAAG | |
| | | TTCCAGGGCAGAGTCACCATGACCGAGGACACATCTACAGACACAGC | |
| | | CTACATGGCGCTGAGCAGCCTGACATCTGAGGACACGGCCGTGTATT | |
| | | ACTGTGCAACAGCCTACGCGTATTACTATGCTTCGGGGGGTTATTATA | |
| | | CCCTTGACTACTGGGGCCAGGGAACCCTGGTCACCGTCTCCTCAGCCT | |
| | | CCACCAAGGGCCCATCGGTCTTCCCCCTGGCACCCTCCTCCAAGAGCA | |
| | | CCTCTGGGGGCACAGCGGCCCTGGGCTGCCTGGTCAAGGACTACTTC | |
| | | CCCGAACCGGTGACGGTGTCGTGGAACTCAGGCGCCCTGACCAGCGG | |
| | | CGTGCACACCTTCCCGGCTGTCCTACAGTCCTCAGGA | |
| | LC-DNA | CAGGCAGGGCTGACTCAGCCACCCTCGGTGTCCAAGGGCTTGAGACA | 1674 |
| | | GACCGCCACACTCACCTGCACTGGGAGCAGCAACAATGTTGGCAACC | |
| | | AAGGAGCAGCTTGGTTGCAGCAGCACCAGGGCCACCCTCCCAAACTC | |
| | | CTATCCTACAGGAATAACAACCGGCCCTCAGGGATCTCAGAGAGATT | |
| | | CTCTGCATCCAGGTCAGGAAACACAGCCTCCCTGACCATTACTGGACT | |
| | | CCAGCCTGAGGACGAGGCAGACTATTACTGCTCAGCATGGGACAGCA | |
| | | GCCTCTCTAATTGGGTGTTCGGCGGAGGGACCAAGCTGACCGTCCTA | |
| | | GGTCAGCCCAAGGCTGCCCCCTCGGTCACTCTGTTCCCGCCCTCCTCT | |
| | | GAGGAGCTTCAAGCCAACAAGGCCACACTGGTGTGTCTCATAAGTGA | |
| | | CTTCTACCCGGGAGCCGTGACAGTGGCCTGGAAGGCAGATAGCAGCC | |
| | | CCGTCAAGGCGGGAGTGGAGACCACCACACCCTCCAAACAAAGCAAC | |
| | | AACAAGTACGCGGCCAGCAGCTA | |
| S24- | HC-DNA | CAGATCACCTTGAAGGAGTCTGGTCCTACGCTGGTGAAACCCACACA | 1675 |
| 223 | | GACCCTCACGCTGACCTGCACCTTCTCTGGGTTCTCACTCAACACTAG | |
| | | TGGAGTGGGTGTGGGCTGGATCCGTCAGCCCCCAGGAAAGGCCCTGG | |
| | | AGTGGCTTGCACTCATTTATTGGGATGATGATAAGCGCTACAGCCCAT | |
| | | CTCTGAAGAGCAGGCTCACCATCACCAAGGACACCTCCAAAAACCAG | |
| | | GTGGTCCTTACAATGACCAACATGGACCCTGTGGACACAGCCACATA | |
| | | TTACTGTGCACACCATACGATTGTTCCAATTTTTGACTACTGGGGCCA | |
| | | GGGAACCCTGGTCACCGTCTCCTCAGGGAGTGCATCCGCCCCAACCCT | |
| | | TTTCCCCCTCGTCTCCTGTGAGAATTCCCCGTCGGATACGAGCAGCGT | |
| | | G | |
| | LC-DNA | CAGTCTGCCCTGACTCAGCCTGCCTCCGTGTCTGGGTCTCCTGGACAG | 1676 |
| | | TCGATCACCATCTCCTGCACTGGAACCAGCAGTGACGTTGGTGGTTAT | |
| | | AACTATGTCTCCTGGTACCAACAACACCCAGGCAAAGCCCCCAAACT | |
| | | CATGATTTATGATGTCAGTAATCGGCCCTCAGGGGTTTCTAATCGCTT | |
| | | CTCTGGCTCCAAGTCTGGCAACACGGCCTCCCTGACCATCTCTGGGCT | |
| | | CCAGGCTGAGGACGAGGCTGATTATTACTGCAACTCATATACAAGCA | |
| | | GCAGCACTCTCGTGGTATTCGGCGGAGGGACCAAGCTGACCGTCCTA | |
| | | GGTCAGCCCAAGGCTGCCCCCTCGGTCACTCTGTTCCCGCCCTCCTCT | |
| | | GAGGAGCTTCAAGCCAACAAGGCCACACTGGTGTGTCTCATAAGTGA | |
| | | CTTCTACCCGGGAGCCGTGACAGTGGCCTGGAAGGCAGATAGCAGCC | |
| | | CCGTCAAGGCGGGAGTGGAGACCACCACACCCTCCAAACAAAGCAAC | |
| | | AACAAGTACGCGGCCAGCAGCTATCTGAGCCTGACGCC | |
| S24- | HC-DNA | CAGGTGCAGCTGCAGGAGTCGGGCCCAGGACTGGTGAAGCCTTCGGA | 1677 |
| 461 | | GACCCTGTCCCTCACGTGCACTGTCTCTGGTGGCTCCATCAGTAGTTA | |
| | | CTACTGGAGCTGGATCCGGCAGCCCCCCGGGAAGGGACTGGAGTGGA | |
| | | TTGGGAATATCTATAACAGTGGGAGCACCAACTACAACCCCTCCCTC | |
| | | AAGAGTCGACTCACCATATCAGTTGACACGTCCAAGAACCACTTCTCC | |
| | | CTGAAGCTGAGCTCTGTGACCGCTGCGGACACGGCCGTGTATTACTGT | |
| | | GCGAGAGGAGGACTAGAGCACGACGGTGACTACGTCTACTACTACGG | |
| | | TATGGACGTCTGGGGCCAAGGGACCACGATCACCGTCTCCTCAGCCT | |
| | | CCACCAAGGGCCCATCGGTCTTCCCCCTGGCACCCTCCTCCAAGAGCA | |
| | | CCTCTGGGGGCACAGCGGCCCTGGGCTGCCTGGTCAAGGACTACTTC | |
| | | CCCGAACCGGTGACGGTGTCGTGGAACTCAGGCGCCCTGACCAGCGG | |
| | | CGTGCACACCTTCCCGGCTGTCCTACAGTCCTCAGGA | |
| | LC-DNA | TCCTATGAGCTGACACAGCCACCCTCGGTGTCAGTGTCCCTAGGACAG | 1678 |
| | | ATGGCCAGGATCACCTGCTCTGGAGAAGCATTGCCAAAAAAATATGC | |
| | | TTATTGGTACCAGCAGAAGCCAGGCCAGTTCCCTATACTGGTGATATA | |
| | | TAAAGACAGCGAGAGGCCCTCAGGGATCCCTGAGCGATTCTCTGGCT | |
| | | CCAGCTCAGGGACAATAGTCACATTGACCATCAGTGGAGTCCAGGCA | |
| | | GAAGACGAGGCTGACTATTACTGTCTATCAGAAGACAGCAGTGGTAC | |
| | | TTGGGTGTTCGGCGGAGGGACCAAGCTGACCGTCCTAGGTCAGCCCA | |
| | | AGGCTGCCCCCTCGGTCACTCTGTTCCCGCCCTCCTCTGAGGAGCTTC | |
| | | AAGCCAACAAGGCCACACTGGTGTGTCTCATAAGTGACTTCTACCCG | |
| | | GGAGCCGTGACAGTGGCCTGGAAGGCAGATAGCAGCCCCGTCAAGGC | |
| | | GGGAGTGGAGACCACCACACCCTCCAAACAAAGCAACAACAAGTAC | |
| | | GCGGCCAGCAGCTA | |
| S24- | HC-DNA | CAGGTGCAGCTGGTGGAGTCTGGGGGAGGCGTGGTCCAGCCTGGGAG | 1679 |
| 511 | | GTCCCTGAGACTCTCCTGTGCAGCCTCTGGATTCACCTTCAGTAGCTA | |
| | | TGGCATGCACTGGGTCCGCCAGGCTCCAGGCAAGGGGCTGGAGTGGG | |
| | | TGGCAGTTATATCATATGATGGAAGTAATAAATACTATGCAGACTCC | |
| | | GTGAAGGGCCGATTCACCATCTCCAGAGACAATTCCAAGAACACGCT | |
| | | GTATCTGCAAATGAACAGCCTGAGAGCTGAGGACACGGCTGTGTATT | |
| | | ACTGTGCGAAATATACGTCAACGGTAACTACGAACTACTACTACGGT | |
| | | ATGGACGTCTGGGGCCAAGGGACCACGGTCACCGTCTCCTCAGCACC | |
| | | CACCAAGGCTCCGGATGTGTTCCCCATCATATCAGGGTGCAGACACC | |
| | | CAAAGGATAACAGCCCTGTGGTCCTGGCATGCTTGATAACTGGGTAC | |
| | | CACC | |
| | LC-DNA | TCCTATGAGCTGACTCAGCCACCCTCAGTGTCCGTGTCCCCAGGACAG | 1680 |
| | | ACAGCCAGCATCACCTGCTCTGGAGATAAATTGGGGGATAAATATGC | |
| | | TTGCTGGTATCAGCAGAAGCCAGGCCAGTCCCCTGTGCTGGTCATCTA | |
| | | TCAAGATAGCAAGCGGCCCTCAGGGATCCCTGAGCGATTCTCTGGCT | |
| | | CCAACTCTGGGAACACAGCCACTCTGACCATCAGCGGGACCCAGGCT | |
| | | ATGGATGAGGCTGACTATTACTGTCAGGCGTGGGACAGCAGCACTGT | |
| | | GGTATTCGGCGGAGGGACCAAGCTGACCGTCCTAGGTCAGCCCAAGG | |
| | | CTGCCCCCTCGGTCACTCTGTTCCCGCCCTCCTCTGAGGAGCTTCAAG | |
| | | CCAACAAGGCCACACTGGTGTGTCTCATAAGTGACTTCTACCCGGGA | |
| | | GCCGTGACAGTGGCCTGGAAGGCAGATAGCAGCCCCGTCAAGGCGGG | |
| | | AGTGGAGACCACCACACCCTCCAAACAAAGCAACAACAAGTACGCG | |
| | | GCCAGCAGCTACC | |
| S24- | HC-DNA | CAGGTGCAGCTGGTGGAGTCTGGGGGAGGCGTGGTCCAGCCTGGGAG | 1681 |
| 788 | | GTCCCTGAGACTCTCCTGTGCAGCGTCTGGATTCACCTTCAGTAGCTA | |
| | | TGGCATGCACTGGGTCCGCCAGGCTCCAGGCAAGGGGCTGGAGTGGG | |
| | | TGGCAGTTATATGGTATGATGGAAGTAATAAATACTATGCAGACTCC | |
| | | GTGAAGGGCCGATTCACCATCTCCAGAGACAATTCCAAGAACACGCT | |
| | | GTATCTGCAAATGAACAGCCTGAGAGCCGAGGACACGGCTGTGTATT | |
| | | ACTGTGCGAGAGGACGTTCCCCAGGTGGGGGCCACTACTACGGTATG | |
| | | GACGTCTGGGGCCAAGGGACCACGGTCACCGTCTCCTCAGGGAGTGC | |
| | | ATCCGCCCCAACCCTTTTCCCCCTCGTCTCCTGTGAGAATTCCCCGTCG | |
| | | GATACGAGCAGCGTG | |
| | LC-DNA | TCCTATGAGCTGACTCAGCCACCCTCAGTGTCCGTGTCCCCAGGACAG | 1682 |
| | | ACAGCCAGCATCACCTGCTCTGGAGATAAATTGGGGGATAAATATGC | |
| | | TTGCTGGTATCAGCAGAAGCCAGGCCAGTCCCCTGTGCTGGTCATCTA | |
| | | TCAAGATAGCAAGCGGCCCTCAGGGATCCCTGAGCGATTCTCTGGCT | |
| | | CCAACTCTGGGAACACAGCCACTCTGACCATCAGCGGGACCCAGGCT | |
| | | ATGGATGAGGCTGACTATTACTGTCAGGCGTGGGACAGCAGCTCTGT | |
| | | GGTATTCGGCGGAGGGACCAAGCTGACCGTCCTAGGTCAGCCCAAGG | |
| | | CTGCCCCCTCGGTCACTCTGTTCCCGCCCTCCTCTGAGGAGCTTCAAG | |
| | | CCAACAAGGCCACACTGGTGTGTCTCATAAGTGACTTCTACCCGGGA | |
| | | GCCGTGACAGTGGCCTGGAAGGCAGATAGCAGCCCCGTCAAGGCGGG | |
| | | AGTGGAGACCACCACACCCTCCAAACAAAGCAACAACAAGTACGCG | |
| | | GCCAGCAGCTA | |
| S24- | HC-DNA | CAGGTCACCTTGAGGGAGTCTGGTCCTGCGCTGGTGAAACCCACACA | 1683 |
| 821 | | GACCCTCACACTGACCTGCACCTTCTCTGGGCTCTCACTCAGCAGTAG | |
| | | TGGAATGTGTGTGAGCTGGATCCGTCAGCCCCCAGGGAAGGCCCTGG | |
| | | AGTGGCTTGCACGCATTGATTGGGATGATGATAAATACTACAGCACA | |
| | | TCTCTGAAGACCAGGCTCACCATCTCCAAGGACACCTCCAAAAATCA | |
| | | GGTGGTCCTTACAATGACCAACATGGACCCTGTGGACACAGCCACGT | |
| | | ATTACTGTGCACGGATATGTACTATGGTTCGGGGACTCCATGATGCTT | |
| | | TTGATATCTGGGGCCAAGGGACAATGGTCACCGTCTCTTCAGGGAGT | |
| | | GCATCCGCCCCAACCCTTTTCCCCCTCGTCTCCTGTGAGAATTCCCCGT | |
| | | CGGATACGAGCAGCGTG | |
| | LC-DNA | GACATCCAGATGACCCAGTCTCCTTCCACCCTGTCTGCATCTGTAGGA | 1684 |
| | | GACAGAGTCACCATCACTTGCCGGGCCAGTCAGAGTATTAGTAGCTG | |
| | | GTTGGCCTGGTATCAGCAGAAACCAGGGAAAGCCCCTAAGCTCCTGA | |
| | | TCTATAAGGCGTCTAGTTTAGAAAGTGGGGTCCCATCAAGGTTCAGC | |
| | | GGCAGTGGATCTGGGACAGAATTCACTCTCACCATCAGCAGCCTGCA | |
| | | GCCTGATGATTTTGCAACTTATTACTGCCAACAGTATAATAGTTATTC | |
| | | GTGGACGTTCGGCCAAGGGACCAAGGTGGAAATCAAACGAACTGTGG | |
| | | CTGCACCATCTGTCTTCATCTTCCCGCCATCTGATGAGCAGTTGAAAT | |
| | | CTGGAACTGCCTCTGTTGTGTGCCTGCTGAATAACTTCTATCCCAGAG | |
| | | AGGCCAAAGTACAGTGGAAGGTGGATAACGC | |
| S144- | HC-DNA | GAGGTGCAGCTGGTGCAGTCTGGAGCAGAGGTGAAAAAGCCCGGGG | 1685 |
| 67 | | AGTCTCTGAAGATCTCCTGTAAGGGTTCTGGATACAGCTTTACCACCT | |
| | | ACTGGATCGCCTGGGTGCGCCAGATGCCCGGGAAAGGCCTGGAGTGG | |
| | | GTGGGGATCATCTATCCTGATGACTCTGATACCAGATACAGCCCGTCC | |
| | | TTCCAAGGCCAGGTCACCATCTCAGCCGACAAGTCCATCGGTACCGC | |
| | | CTACCTGCAGTGGAGTAGCCTGAAGGCCTCGGACACCGCCATGTATT | |
| | | ACTGTGCGAGGGGCCAGTATTACGATTTTTGGAGCGGAGCCGGAGGT | |
| | | GTGGACGTCTGGGGCCAAGGGACCACGGTCACCGTCTCCTCAGCCTC | |
| | | CACCAAGGGCCCATCGGTCTTCCCCCTGGCACCCTCCTCCAAGAGCAC | |
| | | CTCTGGGGGCACAGCGGCCCTGGGCTGCCTGGTCAAGGACTACTTCC | |
| | | CCGAACCGGTGACGGTGTCGTGGAACTCAGGCGCCCTGACCAGCGGC | |
| | | GTGCACACCTTCCCGGCTGTCCTACAGTCCTCAGGA | |
| | LC-DNA | CAGTCTGTGCTGACGCAGCCGCCCTCAGTGTCTGGGGCCCCAGGGCA | 1686 |
| | | GAGGGTCACCATCTCTTGCACTGGGAGCAGGTCCAACATCGGGGCAG | |
| | | GTTATGATGTACAGTGGTACCAGCAGGTTCCAGGAACAGCCCCCAAA | |
| | | CTCCTCATCTCTGGTAACAGCAATCGGCCCTCAGGGGTCCCTGACCGA | |
| | | TTCTCTGGCTCCAAGTCTGGCACCTCAGCCTCCCTGGCCATCACTGGG | |
| | | CTCCAGGCTGAGGATGAGGCTGATTATTACTGCCAGTCCTATGACAGC | |
| | | AGCCTGAGTGGTCTGAGGGTATTCGGCGGAGGGACCAAGCTGACCGT | |
| | | CCTAGGTCAGCCCAAGGCTGCCCCCTCGGTCACTCTGTTCCCGCCCTC | |
| | | CTCTGAGGAGCTTCAAGCCAACAAGGCCACACTGGTGTGTCTCATAA | |
| | | GTGACTTCTACCCGGGAGCCGTGACAGTGGCCTGGAAGGCAGATAGC | |
| | | AGCCCCGTCAAGGCGGGAGTGGAGACCACCACACCCTCCAAACAAAG | |
| | | CAACAACAAGTACGCGGCCAGCAGCTATCTGAGCCTGACGCCTGAGC | |
| | | AGTGGAAGTCCCAC | |
| S144- | HC-DNA | GAGGTGCAGCTGGTGCAGTCTGGAGCAGAGGTGAAAAAGCCCGGGG | 1687 |
| 69 | | AGTCTCTGAAGATCTCCTGTAAGGGTTCTGGATACAGCTTTACCAGCT | |
| | | ACTGGATCGGCTGGGTGCGCCAGATGCCCGGGAAAGGCCTGGAGTGG | |
| | | ATGGGGATCATCTATCCTGGTGACTCTGATACCAGATACAGCCCGTCC | |
| | | TTCCAAGGCCAGGTCACCATCTCAGCCGACAAGTCCATCACTACCGCC | |
| | | TACCTGCAGTGGAGCAGCCTGAAGGCCTCGGACACCGCCATGTATTA | |
| | | CTGTGCGAGGACCCAGACTACGAACTGGTTCGACTCCTGGGGCCAGG | |
| | | GAACCCTGGTCACCGTCTCCTCAGCCTCCACCAAGGGCCCATCGGTCT | |
| | | TCCCCCTGGCACCCTCCTCCAAGAGCACCTCTGGGGGCACAGCGGCC | |
| | | CTGGGCTGCCTGGTCAAGGACTACTTCCCCGAACCGGTGACGGTGTC | |
| | | GTGGAACTCAGGCGCCCTGACCAGCGGCGTGCACACCTTCCCGGCTG | |
| | | TCCTACAGTCCTCAGGA | |
| | LC-DNA | GACATCCAGATGACCCAGTCTCCTTCCACCCTGTCTGTATCTGTAGGA | 1688 |
| | | GACAGAGTCACCATCACTTGCCGGGCCAGTCAGAGTGTTAGTAGCTG | |
| | | GTTGGCCTGGTATCAGCAGAAACCAGGGAAAGCCCCTAAGCTCCTGA | |
| | | TCTATGATGCCTCCAGTTTGGAAAGTGGGGTCCCATCAAGGTTCAGCG | |
| | | GCAGTGGATCTGGGACAGAATTCACTCTCACCATTAGCAGCCTGCAG | |
| | | CCTGATGATTTTGCAACTTATTACTGCCAACAGTATAATAGTTTCTAC | |
| | | ACTTTTGGCCAGGGGACCAAGCTGGAGATCAAACGAACTGTGGCTGC | |
| | | ACCATCTGTCTTCATCTTCCCGCCATCTGATGAGCAGTTGAAATCTGG | |
| | | AACTGCCTCTGTTGTGTGCCTGCTGAATAACTTCTATCCCAGAGAGGC | |
| | | CAAAGTACAGTGGAAGGTGGATAACGCCCTCCAATCGGGTAACTCCC | |
| | | AGGAGAGTGTCACAGAGCAGGACAGCAAGGACAGCACCTACAGCCT | |
| | | CAGCAGCACCCTGACGCTGAGCAAAGCAGACTACGAGAA | |
| S144- | HC-DNA | CAGGTGCAGCTGGTGGAGTCTGGGGGAGGCGTGGTCCAGCCTGGGGG | 1689 |
| 94 | | GTCCCTGAGACTCTCCTGTGCAGCGTCTGGATTCACCTTCAGTAGCTA | |
| | | TGGCATGCACTGGGTCCGCCAGGCTCCAGGCAAGGGGCTGGAGTGGG | |
| | | TGACATTTACACGGTATGATGGAAGTAATAAGTTCTATGCAGACTCCG | |
| | | TGAAGGGCCGATTCTCCATCTCCAGAGACAATTCCAAGAACACGTTG | |
| | | TATCTGCAAATGAATAGTCTGAGAGCTGAGGACACGGCTGTATACTA | |
| | | CTGCGCGAAAGAAAGTCGTGTGGCGTTTGGGGGAGCTATCGCCATCT | |
| | | ACTACTTCGGTATGGACGTCTGGGGCCAAGGGACCACGGTCACCGTC | |
| | | TCCTCAGCCTCCACCAAGGGCCCATCGGTCTTCCCCCTGGCGCCCTGC | |
| | | TCCAGGAGCACCTCTGGGGGCACAGCGGCCCTGGGCTGCCTGGTCAA | |
| | | GGACTACTTCCCCGAACCGGTGACGGTGTCGTGGAACTCAGGCGCCC | |
| | | TGACCAGCGGCGTGCACACCTTCCCGGCTGTCCTACAGTCCTCAGGA | |
| | LC-DNA | GATATTGTGATGACTCAGTCTCCACTCTCCCTGCCCGTCACCCCTGGA | 1690 |
| | | GAGCCGGCCTCCATCTCCTGCAGGTCTAGTCAGAGCCTCCTGCATAGT | |
| | | AATGGATACAACTATTTGGATTGGTACCTGCAGAAGCCAGGGCAGTC | |
| | | TCCACAGCTCCTGATCTATTTGGGTTCTAATCGGGCCTCCGGGGTCCC | |
| | | TGACAGGTTCAGTGGCAGTGGATCAGGCACAGATTTTACACTGAAAA | |
| | | TCAGCAGAGTGGAGGCTGAGGATGTTGGGGTTTATTACTGCATGCAA | |
| | | GCTCTACAAACTCCTCAGTACACTTTTGGCCAGGGGACCAAGCTGGA | |
| | | GATCAAACGAACTGTGGCTGCACCATCTGTCTTCATCTTCCCGCCATC | |
| | | TGATGAGCAGTTGAAATCTGGAACTGCCTCTGTTGTGTGCCTGCTGAA | |
| | | TAACTTCTATCCCAGAGAGGCCAAAGTACAGTGGAAGGTGGATAACG | |
| | | CCCTCCAATCGGGTAACTCCCAGGAGAGTGTCACAGAGCAGGACAGC | |
| | | AAGGACAGCACCTACAGCCTCAGCAGCACCCTGACGCTGAGCAAAGC | |
| | | AGACTACGAGAA | |
| S144- | HC-DNA | GAGGTGCAGTTATTGGAGTCTGGGGGAGGCTTGGTACAGCCTGGGGG | 1691 |
| 113 | | GTCCCTGAGACTCTCCTGTGCAGCCTCTGGATTCACCTTTAGCAACTA | |
| | | TGCCATGAGCTGGGTCCGCCAGGCTCCAGGGAAGGGGCTGGAGTGGG | |
| | | TCTCAGCTATTCGTAATAGTGGTAGTAGCACATACTATGCTGACTCCG | |
| | | TGAAGGGCCGGTTCACCATCTCCAGAGACAATTCCAAGAACACGCTG | |
| | | TATCTGCAAATGAACAGCCTGAGAGCCGAGGACTCGGCCGTATATTA | |
| | | CTGTGCGAAAGTAGGGGGGACAGCAGCTGGTCATCCGTTTTATGACT | |
| | | ACTGGGGCCAGGGAACCCTGGTCACCGTCTCCTCAGCCTCCACCAAG | |
| | | GGCCCATCGGTCTTCCCCCTGGCACCCTCCTCCAAGAGCACCTCTGGG | |
| | | GGCACAGCGGCCCTGGGCTGCCTGGTCAAGGACTACTTCCCCGAACC | |
| | | GGTGACGGTGTCGTGGAACTCAGGCGCCCTGACCAGCGGCGTGCACA | |
| | | CCTTCCCGGCTGTCCTACAGTCCTCAGGACTC | |
| | LC-DNA | GACATCCAGATGACCCAGTCTCCATCCTCCCTGTCTGCATCTGTAGGA | 1692 |
| | | GACAGAGTCACCATCACTTGCCGGGCAAGTCAGAGCATTAGCAACTA | |
| | | TTTAAATTGGTATCAGCAGAAACCAGGGAAAGCCCCTGACCTCCTGA | |
| | | TCTATGCTGCATCCAGTTTGCAAAGTGGGGTCCCATTAAGGTTCAGTG | |
| | | GCAGTGGATCTGGGACAGATTTCACTCTCACCATCAGCAGTCTGCAAC | |
| | | CTGAAGATTTTGCAACTTACTACTGTCAACAGACTTACAGTGCCCCCA | |
| | | CTTTCGGCGGAGGGACCAAGGTGGAGATCAAACGAACTGTGGCTGCA | |
| | | CCATCTGTCTTCATCTTCCCGCCATCTGATGAGCAGTTGAAATCTGGA | |
| | | ACTGCCTCTGTTGTGTGCCTGCTGAATAACTTCTATCCCAGAGAGGCC | |
| | | AAAGTACAGTGGAAGGTGGATAACGCCCTCCAATCGGGTAACTCCCA | |
| | | GGAGAGTGTCACAGAGCAGGACAGCAAGGACAGCACCTACAGCCTC | |
| | | AGCAGCACCCTGACGCTGAGCAAAGCAGACTACGAGAA | |
| S144- | HC-DNA | CAGGTGCAGCTGGTGCAGTCTGGGGCTGAGGTGAAGAAGCCTGGGGC | 1693 |
| 175 | | CTCAGTGAAGGTCTCCTGCAAGGCTTCTGGATACACCTTCACCGGCTA | |
| | | CTATATGCACTGGGTGCGACAGGCCCCTGGACAAGGTCTTGAGTGGA | |
| | | TGGGACGGATCAACCCTAACAGTGGTGGCACAAACTTTGCACAGAGG | |
| | | TTTCAGGGCAGGGTCTCCATGACCAGGGACACCTCCATCAGCACAGC | |
| | | CTACATGGAACTGAGCAGCCTGAGATCTGACGACACGGCCGTATATT | |
| | | ACTGTGCGAGAGGCGCAAAATTCGAGCACCTCCCTTTTGATATCTGGG | |
| | | GCCAAGGGACAATGGTCACCGTCTCTTCAGCCTCCACCAAGGGCCCA | |
| | | TCGGTCTTCCCCCTGGCACCCTCCTCCAAGAGCACCTCTGGGGGCACA | |
| | | GCGGCCCTGGGCTGCCTGGTCAAGGACTACTTCCCCGAACCGGTGAC | |
| | | GGTGTCGTGGAACTCAGGCGCCCTGACCAGCGGCGTGCACACCTTCC | |
| | | CGGCTGTCCTACAGTCCTCAGGA | |
| | LC-DNA | CAGTCTATGCTGACTCAGCCACCCTCAGCGTCTGGGACCCCCGGGCA | 1694 |
| | | GAGGGTCACCATCTCTTGTTCTGGAAGCAGCTCCAACATCGGAAGTA | |
| | | ATTATGTATACTGGTACCAGCAGCTCCCAGGAACGGCCCCCAAACTC | |
| | | CTCATCTATAGGAATAATCAGCGGCCCTCAGGGGTCCCTGACCGATTC | |
| | | TCTGGCTCCAAGTCTGGCACCTCAGCCTCCCTGGCCATCAGTGGGCTC | |
| | | CGGTCCGAGGATGAGGCTGATTATTACTGTGCAGCATGGGATGACAG | |
| | | ACGTTGGGTGTTCGGCGGAGGGACCAAGCTGACCGTCCTAGGTCAGC | |
| | | CCAAGGCTGCCCCCTCGGTCACTCTGTTCCCACCCTCCTCTGAGGAGC | |
| | | TTCAAGCCAACAAGGCCACACTGGTGTGTCTCATAAGTGACTTCTACC | |
| | | CGGGAGCCGTGACAGTGGCCTGGAAGGCAGATAGCAGCCCCGTCAAG | |
| | | GCGGGAGTGGAGACCACCACACCCTCCAAACAAAGCAACAACAAGT | |
| | | ACGCGGCCAGCAGCTA | |
| S144- | HC-DNA | CAGGTGCAACTGGTGCAGTCTGGGGCTGAGGTGAAGAAGCCTGGGGC | 1695 |
| 208 | | CTCAGTGAAGGTCTCCTGCAAGTCTTCTGGATACACCTTCACCGGCTA | |
| | | CTATATGCACTGGGTGCGACAGGCCCCTGGACAAGGGCTTGAGTGGA | |
| | | TGGGACGGATCAACCCTAATAGTGGTGGCACAAACTATGCACAGAAG | |
| | | TTTCAGGGCAGGGTCACCATGACCAGGGACACGTCCATCAGCACAGC | |
| | | CTACATGGAACTGAGCAGGCTGAGATCTGACGACACGGCCGTATATT | |
| | | ACTGTGCGAGAGGGGCCCGAGGTGGCGCGGGGTGCAGTGGCTGGTCA | |
| | | TGTTTTGACTTCTGGGGCCAGGGAACCCTGGTCACCGTCTCCTCAGCC | |
| | | TCCACCAAGGGCCCATCGGTCTTCCCCCTGGCACCCTCCTCCAAGAGC | |
| | | ACCTCTGGGGGCACAGCGGCCCTGGGCTGCCTGGTCAAGGACTACTT | |
| | | CCCCGAACCGGTGACGGTGTCGTGGAACTCAGGCGCCCTGACCAGCG | |
| | | GCGTGCACACCTTCCCGGCTGTCCTACAGTCCTCAGGA | |
| | LC-DNA | CAGTCTGCCCTGACTCAGCCTCGCTCAGTGTCCGGGTCTCCTGGACAG | 1696 |
| | | TCAGTCACTATCTCCTGCACTGGAACCAGCAGTGATGTTGGTGGTTAT | |
| | | AAGTATGTCTCCTGGTACCAACAGCACCCAGGCAAAGCCCCCAAACT | |
| | | CATGATTTATGACGTCAGTAAGCGGCCCTCAGGGGTCCCTGATCGCTT | |
| | | CTCTGGCTCCAAGTCTGGCAACACGGCCTCCCTGACCATCTCTGGGCT | |
| | | CCAGGCTGAGGATGAGGGTGATTATTACTGCTGCTCATATGCAGGCA | |
| | | CCTACAGTTTGGTATTCGGCGGAGGGACCAAGGTGACCGTGACCGTC | |
| | | CTAGGTCAGCCCAAGGCTGCCCCCTCGGTCACTCTGTTCCCGCCCTCC | |
| | | TCTGAGGAGCTTCAAGCCAACAAGGCCACACTGGTGTGTCTCATAAG | |
| | | TGACTTCTACCCGGGAGCCGTGACAGTGGCCTGGAAGGCAGATAGCA | |
| | | GCCCCGTCAAGGCGGGAGTGGAGACCACCACACCCTCCAAACAAAGC | |
| | | AACAACAAGTACGCGGCCAGCAGCTATCTGAGCCTGACGCCTGAGCA | |
| | | GTGGAAGTCCCACA | |
| S144- | HC-DNA | GAGGTGCAGCTGGTGGAGTCTGGGGGAGGCCTGGTCAAGCCGGGGG | 1697 |
| 339 | | GGTCCCTGAGACTCTCCTGTGCAGCCTCTGGATTCACCTTCAGTGACT | |
| | | ATACCATGAACTGGGTCCGACAGGCTCCAGGGAAGGGACTGGAGTGG | |
| | | GTCTCATCCATTACTAGAAGTAGTACTTACATCTACTACGCAGACTCA | |
| | | GTGAAGGGCCGATTCACCATCTCCAGAGACAACGCCAAGAACTCACT | |
| | | GTATCTGCAAATGAACAGCCTGAGAGCCGAGGACACGGCTGTCTATT | |
| | | ACTGTGCGAGAGACCCCTATTACGATATTTTGACTGGTTATTGGAACT | |
| | | ACTGGGGCCAGGGAACCCTGGTCACCGTCTCCTCAGCCTCCACCAAG | |
| | | GGCCCATCGGTCTTCCCCCTGGCACCCTCCTCCAAGAGCACCTCTGGG | |
| | | GGCACAGCGGCCCTGGGCTGCCTGGTCAAGGACTACTTCCCCGAACC | |
| | | GGTGACGGTGTCGTGGAACTCAGGCGCCCTGACCAGCGGCGTGCACA | |
| | | CCTTCCCGGCTGTCCTACAGTCCTCAGGA | |
| | LC-DNA | GAAATTGTGTTGACGCAGTCTCCAGGCACCCTGTCTTTGTCTCCAGGG | 1698 |
| | | GAAAGAGCCACCCTCTCCTGCAGGGCCAGTCAGAGTCTTAGCAGCAG | |
| | | CTACTTAGCCTGGTACCAGCAGAAACCTGGCCAGTCTCCCAGGCTCCT | |
| | | CATTTATGGTGCATCCAGCAGGGCCACTGGCATCCCAGACAGGTTCA | |
| | | GTGGCAGTGGGTCTGGGACAGACTTCACTCTCACCATCAACAGACTG | |
| | | GAGCCTGAAGATTTTGCAGTATATTACTGTCAGCAGTATCGTACCTCA | |
| | | CCTCGAGGCACTTTCGGCGGAGGGACCAAGGTGGAGATCAAACGAAC | |
| | | TGTGGCTGCACCATCTGTCTTCATCTTCCCGCCATCTGATGAGCAGTT | |
| | | GAAATCTGGAACTGCCTCTGTTGTGTGCCTGCTGAATAACTTCTATCC | |
| | | CAGAGAGGCCAAAGTACAGTGGAAGGTGGATAACGCCCTCCAATCGG | |
| | | GTAACTCCCAGGAGAGTGTCACAGAGCAGGACAGCAAGGACAGCAC | |
| | | CTACAGCCTCAGCAGCACCCTGACGCTGAGCAAAGCAGACTACGAGA | |
| | | A | |
| S144- | HC-DNA | GAGGTGCAGCTGGTGGAGTCTGGGGGAGGCTTGGTACAGCCTGGGGG | 1699 |
| 359 | | GTCCCTGAGACTCTCCTGTGCAGCCTCTGGATTCACCTTTAGCAGCTA | |
| | | TGCCATGAGCTGGGTCCGCCAGGCTCCAGGGAAGGGGCTGGAGTGGG | |
| | | TCTCATCTATTAGAGGTAGTGGTGGTAGCACATACTACGCAGACTCCG | |
| | | TGAAGGGCCGGTTCACCATCTCCAGAGACAACTCCAAGTACACGTTG | |
| | | TATCTGCAAATGAACAGCCTGAGAGCCGAGGACACGGCCGTATATTA | |
| | | CTGTGCGAAAATAACTGGAGCCGTCGGGGGGGAGAACTGGTTCGACC | |
| | | CCTGGGGCCAGGGAACCCTGGTCACCGTCTCCTCAGCCTCCACCAAG | |
| | | GGCCCATCGGTCTTCCCCCTGGCGCCCTGCTCCAGGAGCACCTCTGGG | |
| | | GGCACAGCGGCCCTGGGCTGCCTGGTCAAGGACTACTTCCCCGAACC | |
| | | GGTGACGGTGTCGTGGAACTCAGGCGCCCTGACCAGCGGCGTGCACA | |
| | | CCTTCCCGGCTGTCCTACAGTCCTCAGGA | |
| | LC-DNA | GACATCCAGATGACCCAGTCTCCATCCTCCCTGTCTGCATCTGTAGGA | 1700 |
| | | GACAGAGTCACCATCACTTGCCGGGCAAGTCAGAGCATTAGCAGCTA | |
| | | TTTAAATTGGTATCAGCAGAAACCAGGGAAAGCCCCTAAGCTCCTGA | |
| | | TCTATGCTGCATCCAGTTTGCAAAGTGGGGTCCCATCAAGGTTCAGTG | |
| | | GCAGTGGATCTGGGACAGATTTCACTCTCACCATCAGCAGTCTGCAAC | |
| | | CTGAAGATTTTGCAATTTACTACTGTCAACAGACTTCCCGTACCCCGC | |
| | | TCACTTTCGGCGGAGGGACCAAGGTGGAGGTCAAACGAACTGTGGCT | |
| | | GCACCATCTGTCTTCATCTTCCCGCCATCTGATGAGCAGTTGAAATCT | |
| | | GGAACTGCCTCTGTTGTGTGCCTGCTGAATAACTTCTATCCCAGAGAG | |
| | | GCCAAAGTACAGTGGAAGGTGGATAACGCCCTCCAATCGGGTAACTC | |
| | | CCAGGAGAGTGTCACAGAGCAGGACAGCAAGGACAGCACCTACAGC | |
| | | CTCAGCAGCACCCTGACGCTGAGCAAAGCAGACTACGAGAA | |
| S144- | HC-DNA | GAGGTGCGCCTGGTGCAGTCTGGGGGAGGCTTGGTAAAGCCCGGGGG | 1701 |
| 460 | | GTCCCTGAGACTCTCCTGTGCAGCCTCTGGATTCACCTTCAGCACCGC | |
| | | CTGGGTGAGGTGGGTCCGCCAGGCTCCAGGGAAGGGGCTGGAGTGCG | |
| | | TTGGCCGAATCAAAAGTAAAAATGACGGTGACAGAGCAGAGTACGCT | |
| | | GCACCCGCGAGAGGCAGATTCATCATCTCAAGAGATGATGCAGAAAA | |
| | | CATTCTGTATTTACAAATGAACAACCTGAAAACCGAGGACACAGCCT | |
| | | TTTATTACTGTACCACGGATCAAGGAAATAGTAGTGCCTTCTACAGTG | |
| | | CTGACTATTGGGGCCAGGGAACCCTGGTCACCGTCTCCTCAGCATCCC | |
| | | CGACCAGCCCCAAGGTCTTCCCGCTGAGCCTCGACAGCACCCCCCAA | |
| | | GATGGGAACGTGGTCGTCGCATGCCTGGTCCAGGGCTTCTTCCCCCAG | |
| | | GAGCCACTCAGTGTGACCTGGAGCGAAAGCGGACAGAACGTGACCGC | |
| | | CAGAAACTTCCC | |
| | LC-DNA | GACATCCAGATGACCCAGTCTCCATCTGCCATGTCTGCATCTGTAGGA | 1702 |
| | | GACAGAGTCACCATCACTTGTCGGGCGAGTCAGGACATTAACACCTT | |
| | | TTTAACGTGGTTTCAGCAGAAACCAGGAAAAGTCCCTCAGCGCCTGA | |
| | | TCTTTGCTGCATATCGTTTGCAAAGTGGGGTCCCTTCAAGGTTCAGTG | |
| | | GCAGTGGATCTGGGACAGAATTCACTCTCACAATCAACAGCCTGCAG | |
| | | CCTGAAGATGTTGCGACTTATTATTGTCTACACCATAAAACTTATCCG | |
| | | TACACTTTTGGCCAGGGGACCAAACTGGAGATCAAACGAACTGTGGC | |
| | | TGCACCATCTGTCTTCATCTTCCCGCCATCTGATGAGCAGTTGAAATC | |
| | | TGGAACTGCCTCTGTTGTGTGCCTGCTGAATAACTTCTATCCCAGAGA | |
| | | GGCCAAAGTACAGTGGAAGGTGGATAACGCCCTCCAATCGGGTAACT | |
| | | CCCAGGAGAGTGTCACAGAGCAGGACAGCAAGGACAGCACCTACAG | |
| | | CCTCAGCAGCACCCTGACGCTGAGCAAAGCAGACTACGAGAA | |
| S144- | HC-DNA | GAGGTGCAGCTGGTGCAGTCTGGAGCAGAGGTGAAAAAGCCCGGGG | 1703 |
| 466 | | AGTCTCTGAAGATCTCCTGTAAGGGTTCTGGATACAGGTTTACCAGAT | |
| | | ACTGGATCGGCTGGGTGCGCCAGATGCCCGGGAAAGGCCTGGAGTGG | |
| | | ATGGGGATCATCTATCTTGGTGACTCTGAAACCAGATACAGTCCGTCC | |
| | | TTCCAAGGCCAGGTCACCATCTCAGCCGACAACTCCATCAGCACCGC | |
| | | CTACCTGCAGTGGAGCAGCCTGAAGGCCTCGGACACCGCCATGTATT | |
| | | ACTGTGCGAGAAGTTCCAATTGGAATTACGGTGACTACTGGGGCCAG | |
| | | GGAACCCTGGTCACCGTCTCCTCAGCTTCCACCAAGGGCCCATCGGTC | |
| | | TTCCCCCTGGCGCCCTGCTCCAGGAGCACCTCTGGGGGCACAGCGGC | |
| | | CCTGGGCTGCCTGGTCAAGGACTACTTCCCCGAACCGGTGACGGTGTC | |
| | | GTGGAACTCAGGCGCCCTGACCAGCGGCGTGCACACCTTCCCGGCTG | |
| | | TCCTACAGTCCTCAGGA | |
| | LC-DNA | GACATCCAGATGACCCAGTCTCCTTCCACCCTGTCTGCATCTGTGGGA | 1704 |
| | | GACAGAGTCACCATCACTTGCCGGGCCAGTCAGAGTATTACTAGTTG | |
| | | GTTGGCCTGGTATCAGCAGAAATCAGGGAAAGCCCCTAAACTCCTGA | |
| | | TCTATGATGCCTCCAGTTTGGAAAGTGGGGTCCCATCAAGGTTCAGCG | |
| | | GCAGTGGATCTGGGACAGAATTCACTCTCACCATCAGCAGCCTGCAG | |
| | | CCTGATGATTTTGCAACTTATTACTGCCAACAGTATAATAGTTATCCT | |
| | | TGGACGTTCGGCCAAGGGACCAAGGTGGAAATCAAACGAACTGTGGC | |
| | | TGCACCATCTGTCTTCATCTTCCCGCCATCTGATGAGCAGTTGAAATC | |
| | | TGGAACTGCCTCTGTTGTGTGCCTGCTGAATAACTTCTATCCCAGAGA | |
| | | GGCCAAAGTACAGTGGAAGGTGGATAACGCCCTCCAATCGGGTAACT | |
| | | CCCAGGAGAGTGTCACAGAGCAGGACAGCAAGGACAGCACCTACAG | |
| | | CCTCAGCAGCACCCTGACGCTGAGCAAAGCAGACTACGAGAA | |
| S144- | HC-DNA | CAGGTGCAGCTGCAGGAGTCGGGCCCAGGACTGGTGAAGCCTTCGGA | 1705 |
| 469 | | GACCCTGTCCCTCACCTGCACTGTCTCTGGTGGCTCCATCAGTAGTGA | |
| | | CTACTGGAGCTGGATCCGGCAGCCCCCAGGGAAGGGACTGGAGTGGA | |
| | | TTGGATATATGTATTACAGTGGGAGCACCAACTACAACCCCTCCCTCA | |
| | | AGAGTCGAGTCACCATATCAGTAGACACGTCCAAGAACCAGTTCTCC | |
| | | CTGAAGCTGAGCTCTGTGACCGCTGCGGACACGGCCGTGTATTACTGT | |
| | | GCGAGATGGGATAGGGGAAGCAGGCCTCACTACTACTACTATGGTAT | |
| | | GGACGTCTGGGGCCAAGGGACCACGGTCACCGTCTCCTCAGCCTCCA | |
| | | CCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCC | |
| | | GAACCGGTGACGGTGTCGTGGAACTCAGGCGCCCTGACCAGCGGCGT | |
| | | GCACACCTTCCCGGCTGTCCTACAGTCCTCAGGA | |
| | LC-DNA | GATATTGTGATGACTCAGTCTCCACTCTCCCTGCCCGTCACCCCTGGA | 1706 |
| | | GAGCCGGCCTCCATCTCCTGCAGGTCTAGTCAGAGCCTCCTGCATAGT | |
| | | AATGGATACAACTATTTGGATTGGTACCTGCAGAAGCCAGGGCAGTC | |
| | | TCCACAGCTCCTGATCTATTTGGGTTCTAATCGGGCCTCCGGGGTCCC | |
| | | TGACAGGTTCAGTGGCAGTGCATCAGGCACAGATTTTACACTGAAAA | |
| | | TCAGCAGAGTGGAGGCTGAGGATGTTGGGGTTTATTACTGCATGCAA | |
| | | GCTCTACAAGCTTTCACTTTCGGCCCTGGGACCAAAGTGGATATCAAA | |
| | | CGAACTGTGGCTGCACCATCTGTCTTCATCTTCCCGCCATCTGATGAG | |
| | | CAGTTGAAATCTGGAACTGCCTCTGTTGTGTGCCTGCTGAATAACTTC | |
| | | TATCCCAGAGAGGCCAAAGTACAGTGGAAGGTGGATAACGCCCTCCA | |
| | | ATCGGGTAACTCCCAGGAGAGTGTCACAGAGCAGGACAGCAAGGAC | |
| | | AGCACCTACAGCCTCAGCAGCACCCTGACGCTGAGCAAAGCAGACTA | |
| | | CGAGA | |
| S144- | HC-DNA | GAGGTGCAGCTGGTGCAGTCTGGAGCAGAGGTGAAAAAGCCCGGGG | 1707 |
| 509 | | AGTCTCTGAAGATCTCCTGTAAGGGTTCTGCATACACCTTTACCACCT | |
| | | ACTGGATCGGCTGGGTGCGCCAGATGCCCGGGAAAGGCCTGGAGTGG | |
| | | ATGGGGATCATCTATCCTGGTGACTCTGATACCAGATACAGCCCGTCC | |
| | | TTCCAAGGCCAGGTCACCATCTCAGCCGACAAGTCCATCAGCACCGC | |
| | | CTACCTGCAGTGGAGCAGCCTGAAGGCCTCGGACACCGCCATGTATT | |
| | | ACTGTGCGAGATTATTATTGGTGGCTGGTCCCTTTGACTACTGGGGCC | |
| | | AGGGAACCCTGGTCACCGTCTCCTCAGCCTCCACCAAGGGCCCATCG | |
| | | GTCTTCCCCCTGGCACCCTCCTCCAAGAGCACCTCTGGGGGCACAGCG | |
| | | GCCCTGGGCTGCCTGGTCAAGGACTACTTCCCCGAACCGGTGACGGT | |
| | | GTCGTGGAACTCAGGCGCCCTGACCAGCGGCGTGCACACCTTCCCGG | |
| | | CTGTCCTACAGTCCTCAGGACTCTACTCCCTCAGCAGCGTGGTGACCG | |
| | | TGCCCTCCAGCAGCTTGGGCACCCAGACCTACATCTGCAACGTGAATC | |
| | | ACAAGCCCAGCAACACCAAGGTGGACA | |
| | LC-DNA | GACATCCAGATGACCCAGTCTCCTTCCACCCTGTCTGCATCTGTAGGA | 1708 |
| | | GACAGAGTCACCATCACTTGCCGGGCCAGTCAGAGTATTAGTAGCTG | |
| | | GTTGGCCTGGTATCAGCAGAAACCAGGGAAAGCCCCTAACCTCCTGA | |
| | | TCTATGATGCCTCCAGTTTGGAAAGTGGGGTCCCATCAAGGTTCAGCG | |
| | | GCAGTGGATCTGGGACAGAATTCACTCTCACCATCAGCAGCCTGCAG | |
| | | CCTGATGATTTTGCAACTTATTACTGCCAACAGTATAATAGTTATCCG | |
| | | TGGACGTTCGGCCAAGGGACCAAGGTGGAAATCAAACGAACTGTGGC | |
| | | TGCACCATCTGTCTTCATCTTCCCGCCATCTGATGAGCAGTTGAAATC | |
| | | TGGAACTGCCTCTGTTGTGTGCCTGCTGAATAACTTCTATCCCAGAGA | |
| | | GGCCAAAGTACAGTGGAAGGTGGATAACGC | |
| S144- | HC-DNA | CAGGTGCAGCTGCTGCAGTCTGGGGCTGAAGTGAAGAAGCCTGGGGC | 1709 |
| 516 | | CTCAGTGAAGGTCTCCTGCAAGGCTTCTGGATACACCTTCACCGGCTA | |
| | | CTATATGCACTGGGTGCGACAGGCCCCTGGACAAGGGCTTGAGTGGA | |
| | | TGGGACGGATCAACCCTAACAGTGGTGGCACAAATTATGCACAGAAG | |
| | | TTTCAGGGCAGGGTCACCATGACCAGGGACACGTCCATCAGCACAGC | |
| | | CTACATGGAGCTGAGCAGGCTGACATCTGACGACACGGCCGTGTATT | |
| | | ACTGTGCGACCAAAACTGGAATTGATCGCTACTACTACTACTACATGG | |
| | | ACGTCTGGGGCAAAGGGACCACGGTCACCGTCTCCTCAGCCTCCACC | |
| | | AAGGGCCCATCGGTCTTCCCCCTGGCACCCTCCTCCAAGAGCACCTCT | |
| | | GGGGGCACAGCGGCCCTGGGCTGCCTGGTCAAGGACTACTTCCCCGA | |
| | | ACCGGTGACGGTGTCGTGGAACTCAGGCGCCCTGACCAGCGGCGTGC | |
| | | ACACCTTCCCGGCTGTCCTACAGTCCTCAGGA | |
| | LC-DNA | CAGTCTGTGCTGACGCAGCCGCCCTCAGTGTCTGAGGCCCCAGGGCA | 1710 |
| | | GAGGGTCACCATCTCCTGCACTGGGAGCAGCTCCAACATCGGGGCAG | |
| | | GTTATGATGTACACTGGTACCAGCAGCTTCCAGGAACAGCCCCCAAA | |
| | | CTGCTCATCTATGGTAACATTAATCGGCCCTCAGGGGTCCCTGACCGA | |
| | | TTCTCTGGCTCCAAGTCTGGCACCTCAGCCTCCCTGGCCATCACTGGG | |
| | | CTCCAGGCTGAGGATGAGGCTGATTATTACTGCCAGTCCTATGACAAC | |
| | | AGCCTGAATGGTTCGGTGTTCGGCGGAGGGACCAAACTGACCGTCCT | |
| | | ACGTCAGCCCAAGGCTGCCCCCTCGGTCACTCTGTTCCCACCCTCCTC | |
| | | TGAGGAGCTTCAAGCCAACAAGGCCACACTGGTGTGTCTCATAAGTG | |
| | | ACTTCTACCCGGGAGCCGTGACAGTGGCCTGGAAGGCAGATAGCAGC | |
| | | CCCGTCAAGGCGGGAGTGGAGACCACCACACCCTCCAAACAAAGCAA | |
| | | CAACAAGTACGCGGCCAGCAGCTA | |
| S144- | HC-DNA | CAGGTGCAGCTGCAGGAGTCGGGCCCAGGACTGGTGAAGCCTTCGGA | 1711 |
| 568 | | GACCCTGTCCCTCACCTGCAGTGTCTCTGGTGGCTCCATCAGTGATTA | |
| | | CTACTGGAGCTGGATCCGGCAGCCCCCTGGGAAGGGACTGGAGTGGA | |
| | | TTGGATATATCTATAACAGTGGGAGTACCAACTACAACCCCTCCCTCA | |
| | | AGAGTCGAGTCACCATATCAGCAGACCCGTCCAAGAACCAGTTCTCC | |
| | | CTGAAGTTGAGCTCTGTGACCGCCGCAGACACGGCCGTATATTACTGT | |
| | | GCGAGACCTCACGGCGGTGACTACGCTTTTGATATTTGGGGCCAAGG | |
| | | GACAATGGTCACCGTCTCTTCAGCATCCCCGACCAGCCCCAAGGTCTT | |
| | | CCCGCTGAGCCTCGACAGCACCCCCCAAGATGGGAACGTGGTCGTCG | |
| | | CATGCCTGGTCCAGGGCTTCTTCCCCCAGGAGCCACTCAGTGTGACCT | |
| | | GGAGCGAAAGCGGACAGAACGTGACCGCCAGAAACTTCCC | |
| | LC-DNA | GAAATTGTGTTGACGCAGTCTCCAGGCACCCTGTCTTTGTCTCCAGGG | 1712 |
| | | GAAAGAGCCACCCTCTCATGTAGGGCCAGTCAGAGTGTTAGCAGCAA | |
| | | CTTCCTAGCCTGGTACCAGCAGAAACCTGGCCAGCCTCCCAGGCTCCT | |
| | | CATCTATGGTGCATCCGTCAGGGCCACTGGCATCCCAGACAGGTTCA | |
| | | GTGGCAGTGGGTCTGGGACAGACTTCACTCTCACCATCACCAGACTG | |
| | | GAGCCTGAAGATTTTGCAGTATATTACTGTCAGCAGTATGGTAGCTTA | |
| | | CCTCGGACGTTCGGCCAAGGGACCAAGGTGGAAATCAAACGAACTGT | |
| | | GGCTGCACCATCTGTCTTCATCTTCCCGCCATCTGATGAGCAGTTGAA | |
| | | ATCTGGAACTGCCTCTGTTGTGTGCCTGCTGAATAACTTCTATCCCAG | |
| | | AGAGGCCAAAGTACAGTGGAAGGTGGATAACGCCCTCCAATCGGGTA | |
| | | ACTCCCAGGAGAGTGTCACAGAGCAGGACAGCAAGGACAGCACCTA | |
| | | CAGCCTCAGCAGCACCCTGACGCTGAGCAAAGCAGACTACGAGA | |
| S144- | HC-DNA | CAGGTCCAGCTGGTGCAATCTGGGGCTGAGGTGATGAAGCCTGGGTC | 1713 |
| 576 | | CTCGGTGAAGGTCTCCTGCAAGGCTTCTGGAGGCACCTTCAGCAGCTA | |
| | | TAGTATCACCTGGGTGCGACAGGCCCCTGGACAAGGGCTTGAGTGGA | |
| | | TGGGAAGGATCATCCCTATCCTTGGTATAGCAAACTACGCACAGAAG | |
| | | TTCCAGGGCAGAGTCACGATTACCGCGGACAAATCCACGAGCACAGC | |
| | | CTACATGGAGCTGAGCAGCCTGAGATCTGAGGACACGGCCGTGTATT | |
| | | ACTGTGCGAGAGGGTATAGTGGGAGCCCCTCGAATTTAGACGGTATG | |
| | | GACGTCTGGGGCCAAGGGACCACGGTCACCGTCTCCTCAGCCTCCAC | |
| | | CAAGGGCCCATCGGTCTTCCCCCTGGCACCCTCCTCCAAGAGCACCTC | |
| | | TGGGGGCACAGCGGCCCTGGGCTGCCTGGTCAAGGACTACTTCCCCG | |
| | | AACCGGTGACGGTGTCGTGGAACTCAGGCGCCCTGACCAGCGGCGTG | |
| | | CACACCTTCCCGGCTGTCCTACAGTCCTCAGGA | |
| | LC-DNA | CATCCAGATGACCCAGTCTCCTTCCACCCTGTCTGCATCTGTAGGAGA | 1714 |
| | | CAGAGTCACCATCACTTGCCGGGCCAGTCAGAGTATTAGTAGCTGGTT | |
| | | GGCCTGGTATCAGCAGAAACCAGGGAAAGCCCCTAAGCTCCTGATCT | |
| | | ATGATGCCTCCAGTTTGCAAAGTGGGGTCCCATCAAGGTTCAGCGGC | |
| | | AGTGGATCTGGGACAGAATTCACTCTCACCATCAGCAGCCTGCAGCC | |
| | | TGATGATTTTGCAACTTATTACTGCCAACAGTATAATAGTTATTCTCC | |
| | | GATCACCTTCGGCCAAGGGACACGACTCGAGATTAAACGAACTGTGG | |
| | | CTGCACCATCTGTCTTCATCTTCCCGCCATCTGATGAGCAGTTGAAAT | |
| | | CTGGAACTGCCTCTGTTGTGTGCCTGCTGAATAACTTCTATCCCAGAG | |
| | | AGGCCAAAGTACAGTGGAAGGTGGATAACGCCCTCCAATCGGGTAAC | |
| | | TCCCAGGAGAGTGTCACAGAGCAGGACAGCAAGGACAGCACCTACA | |
| | | GCCTCAGCAGCACCCTGACGCTGAGCAAAGCAGACTACGAGAA | |
| S144- | HC-DNA | CAGCTGCAGCTGCAGGAGTCGGGCCCAGGACTGGTGAAGCCTTCGGA | 1715 |
| 588 | | GACCCTGTCCCTCACCTGCACTGTCTCTGGTGGCTCCATCAGCAGTAG | |
| | | TAGTTACTACTGGGGCTGGATCCGCCAGCCCCCAGGGAAGGGGCTGG | |
| | | AGTGGATTGGGAGTATCTATTATAGTGGGAGCACCTACTACAACCCG | |
| | | TCCCTCAAGAGTCGATTCACCATATCCGTAGACACGTCCAAGAACCA | |
| | | GTTCTCCCTGAAGCTGAGCTCTGTGACCGCCGCAGACACGGCTGTGTA | |
| | | TTACTGTGCGGCCTATCAGAGGAAACTAGGATATTGTCGTGGTAATA | |
| | | GCTGCTTTTCCTGCTTCGACCCCTGGGGCCAGGGAACCCTGGTCACCG | |
| | | TCTCCTCAGCCTCCACCAAGGGCCCATCGGTCTTCCCCCTGGCACCCT | |
| | | CCTCCAAGAGCACCTCTGGGGGCACAGCGGCCCTGGGCTGCCTGGTC | |
| | | AAGGACTACTTCCCCGAACCGGTGACGGTGTCGTGGAACTCAGGCGC | |
| | | CCTGACCAGCGGCGTGCACACCTTCCCGGCTGTCCTACAGTCCTCAGG | |
| | | A | |
| | LC-DNA | TCCTATGAGCTGACTCAGCCACCCTCAGTGTCCGTGTCCCCAGGACAG | 1716 |
| | | ACAGCCAGCATCACCTGCTCTGGAGATAAATTGGGGGATAAATATGC | |
| | | TTGCTGGTATCAGCAAAAGCCAGGCCAGTCCCCTGTGCTGGTCATCTA | |
| | | TCAAGATACCAAGCGGCCCTCAGGGATCCCTGAGCGATTCTCTGGCTC | |
| | | CAACTCTGGGAACACAGCCACTCTGACCATCAGCGGGACCCAGGCTA | |
| | | TGGATGAGGCTGACTATTACTGTCAGGCGTGGGACAGTAGCACTGTG | |
| | | TTATTCGGCGGAGGGACCAAGCTGACCGTCCTAGGTCAGCCCAAGGC | |
| | | TGCCCCCTCGGTCACTCTGTTCCCGCCCTCCTCTGAGGAGCTTCAAGC | |
| | | CAACAAGGCCACACTGGTGTGTCTCATAAGTGACTTCTACCCGGGAG | |
| | | CCGTGACAGTGGCCTGGAAGGCAGATAGCAGCCCCGTCAAGGCGGGA | |
| | | GTGGAGACCACCACACCCTCCAAACAAAGCAACAACAAGTACGCGGC | |
| | | CAGCAGCTATCTGAGCCTGACGCCTGAGCAGTGGAAGTCCCACA | |
| S144- | HC-DNA | GAGGTGCACCTGGTGCAGTCTGGAGCAGAGGTGAAACAGCCCGGGG | 1717 |
| 628 | | AGTCTCTGAAGATCTCCTGTAAGGGTTCTGGATACAACTTTGCCACCT | |
| | | ACTGGATCGCCTGGGTGCGCCAGATGCCCGGGAAAGGCCTGGAGTGG | |
| | | ATGGGGATCATCTATCCTGGTGACTCTGATACCAGATACAGCCCGTCC | |
| | | TTCCAAGGCCAGGTCATCATCTCAGCCGACAAGTCCATCGGCACCGC | |
| | | CTTCCTGCAGTGGAGCAGCCTGAAGGCCTCGGACACCGCCATGTATT | |
| | | ACTGTGCGAGGCGGGGGTATAGTAGCTCTAACTATCGCGTTGACGAA | |
| | | TACTATTACTACGGTATGGACGTCTGGGGCCAAGGGACCACGGTCAC | |
| | | CGTCTCCTCAGCATCCCCGACCAGCCCCAAGGTCTTCCCGCTGAGCCT | |
| | | CTGCAGCACCCAGCCAGATGGGAACGTGGTCATCGCCTGCCTGGTCC | |
| | | AGGGCTTCTTCCCCCAGGAGCCACTCAGTGTGACCTGGAGCGAAAGC | |
| | | GGACAGGGCGTGACCGCCAGAAACTTCCCC | |
| | LC-DNA | CAGTCTGTGCTGACGCAGCCGCCCTCAATGTCTGGGGCCCCAGGGCA | 1718 |
| | | GAGGGTCACCATCTCCTGCACTGGGAGCAGCTCCAACATCGGGGCAG | |
| | | GTTATGATGTACACTGGTACCAGCAGCTTCCAGGAGCAGCCCCCAAA | |
| | | CTCCTCATCTATGGTGACACCAGTCGGCCCTCAGGGGTCCCTGACCGA | |
| | | TTCTCTGGCTCCAAGTCTGACACCTCAGCCTCCCTGGCCATCACTGGG | |
| | | CTCCAGGCTGAGGATGAGGCTGATTATTACTGCCAGTCCTTTGACAGA | |
| | | AGTCTGAGTGGTCTCGTGATTTTCGGCGGAGGGACCAGGCTGACCGT | |
| | | CCTCGGTCAGCCCAAGGCTGCCCCCTCGGTCACTCTGTTCCCACCCTC | |
| | | CTCTGAGGAGCTTCAAGCCAACAAGGCCACACTGGTGTGTCTCATAA | |
| | | GTGACTTCTACCCGGGAGCCGTGACAGTGGCCTGGAAGGCAGATAGC | |
| | | AGCCCCGTCAAGGCGGGAGTGGAGACCACCACACCCTCCAAACAAAG | |
| | | CAACAACAAGTACGCGGCCAGCAGCTAAGATCGGAAGAGC | |
| S144- | HC-DNA | CAGGTGCAGCTGGTGCAGTCTGGGGCTGAGGTGAAGAAGCCTGGGGC | 1719 |
| 740 | | CTCAGTGAAGGTCTCCTGCAAGGCTTCTGGATACACCTTCACCGGCTA | |
| | | CTATATGCACTGGGTGCGACAGGCCCCTGGACAAGGGCTTGAGTGGA | |
| | | TGGGACGGATCAACCCTAACAGTGGTGACACAAACTATGCACAGAAG | |
| | | TTTCAGGGCAGGGTCACCATGACCAGGGACACGTCCATCAGCACAGC | |
| | | CTACATGGAGCTGAGCAGGCTGAGATCTGACGACACGGCCGTGTATT | |
| | | ACTGTGCGAGATTGGGTAAAGGAATGGCAGCAGCCCGTACTGTCTTT | |
| | | GACTCCTGGGGCCAGGGAACCCTGGTCACCGTCTCCTCAGCCTCCACC | |
| | | AAGGGCCCATCGGTCTTCCCCCTGGCACCCTCCTCCAAGAGCACCTCT | |
| | | GGGGGCACAGCGGCCCTGGGCTGCCTGGTCAAGGACTACTTCCCCGA | |
| | | ACCGGTGACGGTGTCGTGGAACTCAGGCGCCCTGACCAGCGGCGTGC | |
| | | ACACCTTCCCGGCTGTCCTACAGTCCTCAGGA | |
| | LC-DNA | GAAGTTGTGTTGACGCAGTCTCCAGGCACCCTGTCTTTGTCTCCAGGG | 1720 |
| | | GAAAGAGCCACCCTCTCCTGCAGGGCCAGTCAGAGTGTTAGCAGCAG | |
| | | CTACTTAGCCTGGTACCAGCAGAAACCTGGCCAGGCTCCCAGGCTCG | |
| | | TCATCTATGGTGCATCCAGCAGGGCCACTGGCATCCCAGACAGGTTC | |
| | | AGTGGCAGTGGGTCTGGGACAGACTTCACTCTCACCATCAGCAGACT | |
| | | GGAGCCTGAAGATTTTGCAGTGTATTACTGTCAGCAGTTTGGTAGCTC | |
| | | TCCCACCTTCGGCCGAGGGACACGACTGGAGATTAAACGAACTGTGG | |
| | | CTGCACCATCTGTCTTCATCTTCCCGCCATCTGATGAGCAGTTGAAAT | |
| | | CTGGAACTGCCTCTGTTGTGTGCCTGCTGAATAACTTCTATCCCAGAG | |
| | | AGGCCAAAGTACAGTGGAAGGTGGATAACGCCCTCCAATCGGGTAAC | |
| | | TCCCAGGAGAGTGTCACAGAGCAGGACAGCAAGGACAGCACCTACA | |
| | | GCCTCAGCAGCACCCTGACGCTGAGCAAAGCAGACTACGAGAA | |
| S144- | HC-DNA | CAGGTGCACCTGGTGCAGTCTGGGGCTGAGGTGAAGAAGCCTGGGGC | 1721 |
| 741 | | CTCAGTGAAGGTCTCCTGCAAGGCTTCTGGATACACCTTCACCGGCTA | |
| | | CTATATGAACTGGGTGCGACAGGCCCCTGGACAAGGGCTTGAGTGGA | |
| | | TGGGACGGATCAACCCTAACAGTGGTGGCACAAACTATGCACAGAAG | |
| | | TTTCAGGGCAGGGTCACCATGACCAGGGACACGTCCATCAGCACAGC | |
| | | CTACATGGAACTGAGCAGGCTGAGATCTGACGACGCGGCCGTGTATT | |
| | | ACTGTGCGAGAGCTGAGAGGTATAGCAGCAGCTGGTACAATCTTTAC | |
| | | TACTGGGGCCAGGGAACCCTGGTCACCGTCTCCTCAGCCTCCACCAA | |
| | | GGGCCCATCGGTCTTCCCCCTGGCACCCTCCTCCAAGAGCACCTCTGG | |
| | | GGGCACAGCGGCCCTGGGCTGCCTGGTCAAGGACTACTTCCCCGAAC | |
| | | CGGTGACGGTGTCGTGGAACTCAGGCGCCCTGACCAGCGGCGTGCAC | |
| | | ACCTTCCCGGCTGTCCTACAGTCCTCAGGA | |
| | LC-DNA | CAGTCTGTGCTGACTCAGCCACCCTCAGCGTCTGGGACCCCCGGGCA | 1722 |
| | | GAGGGTCACCATCTCTTGTTCTGGAAGCAGCTCCAACATCGGAAGTA | |
| | | ATACTGTAAACTGGTACCAGCAGCTCCCAGGAACGGCCCCCAAGCTC | |
| | | CTCATCTATAGTAATAATCAGCGGCCCTCAGGGGTCCCTGACCGATTC | |
| | | TCTGGCTCCAAGTCTGGCACCTCAGCCTCCCTGGCCATCAGTGGGCTC | |
| | | CAGTCTGAGGATGAGGCTGATTATTACTGTGCAGCATGGGATGACAG | |
| | | CCTGAATGGTGTGGTATTCGGCGGAGGGACCAAGCTGACCGTCCTAG | |
| | | GTCAGCCCAAGGCTGCCCCCTCGGTCACTCTGTTCCCGCCCTCCTCTG | |
| | | AGGAGCTTCAAGCCAACAAGGCCACACTGGTGTGTCTCATAAGTGAC | |
| | | TTCTACCCGGGAGCCGTGACAGTGGCCTGGAAGGCAGATAGCAGCCC | |
| | | CGTCAAGGCGGGAGTGGAGACCACCACACCCTCCAAACAAAGCAAC | |
| | | AACAAGTACGCGGCCAGCAGCTATCTGAGCCTGACGCCTGAGCAGTG | |
| | | GAAGTCCCACA | |
| S144- | HC-DNA | GAGGTGCAGCTGGTGCAGTCTGGAGCAGAGGTGAAAAAGCCCGGGG | 1723 |
| 803 | | AGTCTCTGAAGATCTCCTGTAAGGGTTCTAGATACAGCTTTACCAGAT | |
| | | ACTGGATCGCCTGGGTGCGCCAGATGCCCGGGAAAGGCCTGGAGTGG | |
| | | ATGGGGATCATCTATCCTGGTGACTCTGATACCAGATACAGCCCGTCC | |
| | | TTCCAAGGCCCGGTCACCATCTCAGCCGACAAGTCCATCAGCACCGC | |
| | | CTACCTGCAGTGGAGCAGCCTGAAGGCCTCGGACACCGCCATATATT | |
| | | ACTGTGCGAGACTCCCGAACAGTAACTACGTTGACTACTGGGGCCAG | |
| | | GGAACCCTGGTCACCGTCTCCTCAGCCTCCACCAAGGGCCCATCGGTC | |
| | | TTCCCCCTGGCACCCTCCTCCAAGAGCACCTCTGGGGGCACAGCGGC | |
| | | CCTGGGCTGCCTGGTCAAGGACTACTTCCCCGAACCGGTGACGGTGTC | |
| | | GTGGAACTCAGGCGCCCTGACCAGCGGCGTGCACACCTTCCCGGCTG | |
| | | TCCTACAGTCCTCAGGACTCTACTCCCTCAGCAGCGTGGTGACCGTGC | |
| | | CCTCCAGCAGCTTGGGCACCCAGACCTACATCTGCAACGTGAATCAC | |
| | | AAGCCCAGCAACACCAAGGTGGACAA | |
| | LC-DNA | GACATCCAGATGACCCAGTCTCCTTCCACCCTGTCTGCATCTGTAGGA | 1724 |
| | | GACAGAGTCACCATCACTTGCCGGGCCAGTCAGAGTATTAGTAGTTG | |
| | | GTTGGCCTGGTATCAGCAGAAACCAGGGAAAGCCCCTAAGCTCCTGA | |
| | | TCTATGATGCCTCCAGTTTGGAAAGTGGGGTCCCATCAAGGTTCAGCG | |
| | | GCAGTGGATCTGGGACAGAATTCACTCTCACCATCAGCAGCCTGCAG | |
| | | CCTGATGATTTTGCAACTTATTACTGCCAACAGTATAATATTTACCCG | |
| | | TACACTTTTGGCCAGGGGACCAAGCTGGACATCAAACGAACTGTGGC | |
| | | TGCACCATCTGTCTTCATCTTCCCGCCATCTGATGAGCAGTTGAAATC | |
| | | TGGAACTGCCTCTGTTGTGTGCCTGCTGAATAACTTCTATCCCAGAGA | |
| | | GGCCAAAGTACAGTGGAAGGTGGATAACGCCCTCCAATCGGGTAACT | |
| | | CCCAGGAGAGTGTCACAGAGCAGGACAGCAAGGACAGCACCTACAG | |
| | | CCTCAGCAGCACCCTGACGCTGAGCAAAGCAGACTACGAGAA | |
| S144- | HC-DNA | CAGGTGCAGCTGGTGGAGTCTGGGGGAGGCGTGGTCCAGCCTGGGGG | 1725 |
| 843 | | GTCCGTAAGACTCTCCTGTGCAGCGTCTGGATTCGACTTCACTAATAA | |
| | | TGGCATGTATTGGGTCCGCCAGGCTCCAGGCAAGGGGCTGGAGTGGG | |
| | | TGGCATTTATACGGTATGATGGAAATAAACAAGACTATGCAGACTCC | |
| | | GTGAAGGGCCGATTCACCATCTCCAGAGACAATTCCAAAAACACTCT | |
| | | GTATCTGCAAATGAGCAGCCTTAGACCTGAGGACACGGCTGTATATT | |
| | | ACTGTGCGAAAGGTGTTTATACTGAAAATTACGGCTGGGGCCAGGGA | |
| | | ACCCTGGTCACCGTCTCCTCAGGGACCACGGTCACCGTCTCCTCAGCC | |
| | | TCCACCAAGGGCCCATCGGTCTTCCCCCTGGCGCCCTGCTCCAGGAGC | |
| | | ACCTCCGAGAGCACAGCGGCCCTGGGCTGCCTGGTCAAGGACTACTT | |
| | | CCCCGAACCGGTGACGGTGTCGTGGAACTCAGGCGCTCTGACCAGCG | |
| | | GCGTGCACACCTTCCCAGCTGTCCTACAGTCCTCAGGACTCTACTCCC | |
| | | TCAGCAGCGTGGTGACCGTGCCCTCCAGCAACTTCGGCACCCAGACC | |
| | | TACACCTGCAACGTAGATCACAAGCCCAGCAACACCAAGGTGGACAA | |
| | LC-DNA | GAAATTGTGTTGACGCAGTCTCCAGGCACCCTGTCTTTGTCTCCAGGG | 1726 |
| | | GAAAGAGCCACCCTCTCCTGCAGGGCCAGTCAGACTGTTACCAGCAG | |
| | | GTACTTAGCCTGGTATCAGCAGAAGCCTGGCCAGGCTCCCAGGCTCCT | |
| | | CATCTATGGTGCATCCACCAGGGCCACTGGCATCCCAGACAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTATGGTAATTCA | |
| | | CCTCCGTACACTTTTGGCCAGGGGACCAAGCTGGAGATCAAACGAAC | |
| | | TGTGGCTGCACCATCTGTCTTCATCTTCCCGCCATCTGATGAGCAGTT | |
| | | GAAATCTGGAACTGCCTCTGTTGTGTGCCTGCTGAATAACTTCTATCC | |
| | | CAGAGAGGCCAAAGTACAGTGGAAGGTGGATAACGCCCTCCAATCGG | |
| | | GTAACTCCCAGGAGAGTGTCACAGAGCAGGACAGCAAGGACAGCAC | |
| | | CTACAGCCTCAGCAGCACCCTGACGCTGAGCAAAGCAGACTACGAGA | |
| | | A | |
| S144- | HC-DNA | CAGGTGCAGCTGGTGGAGTCTGGGGGAGGCGTGGTCCAGCCTGGGAG | 1727 |
| 877 | | GTCCCTGAGACTCTCCTGTGCAGCCTCTGGATTCACCTTCAGTACCTA | |
| | | TGGCATGCACTGGGTCCGCCAGGCTCCAGGCAAGGGGCTGGAGTGGG | |
| | | TGGCAGTTATATCATATGATGGAAGTAATAAATATTATGCAGACTCCG | |
| | | TGAAGGGCCGATTCACCATCTCCAGAGACAATTCCAAGAACACGCTG | |
| | | TATCTGCAAATGAACAGCCTGAGAGCTGAGGACACGGCTGTGTATTA | |
| | | CTGTGCGAAACAGCAAGGCACCTATTGCAGTGGTGGTAACTGCTACT | |
| | | CGGGATATTTTGACTACTGGGGCCAGGGAACCCTGGTCACCGTCTCCT | |
| | | CAGCCTCCACCAAGGGCCCATCGGTCTTCCCCCTGGCACCCTCCTCCA | |
| | | AGAGCACCTCTGGGGGCACAGCGGCCCTGGGCTGCCTGGTCAAGGAC | |
| | | TACTTCCCCGAACCGGTGACGGTGTCGTGGAACTCAGGCGCCCTGAC | |
| | | CAGCGGCGTGCACACCTTCCCGGCTGTCCTACAGTCCTCAGGACTCTA | |
| | | CTCCCTCAGCAGCGTGGTGACCGTGCCCTCCAGCAGCTTGGGCACCCA | |
| | | GACCTACATCTGCA | |
| | LC-DNA | GACATCCAGATGACCCAGTCTCCATCCTCCCTGTCTGCATCTGTAGGA | 1728 |
| | | GACAGAGTCACCATCACTTGCCAGGCGAGTCAGGACATTAGCAACTA | |
| | | TTTAAATTGGTATCAGCAGAAACCAGGGAAAGCCCCTAAGCTCCTGA | |
| | | TCTACGATGCATCGAATTTGGAAACAGGGGTCCCATCAAGGTTCAGT | |
| | | GGAAGTGGATCTGGGACAGATTTTAGTTTTAGTATCAGCAGCCTGCA | |
| | | GCCTGAAGATATTGCAACATATTACTGTCAACAGTATGATAATGTCCC | |
| | | TCTTACTTTCGGCGGAGGGACCAAGGTGGAGATCAAACGAACTGTGG | |
| | | CTGCACCATCTGTCTTCATCTTCCCGCCATCTGATGAGCAGTTGAAAT | |
| | | CTGGAACTGCCTCTGTTGTGTGCCTGCTGAATAACTTCTATCCCAGAG | |
| | | AGGCCAAAGTACAGTGGAAGGTGGATAACGCCCTCCAATCGGGTAAC | |
| | | TCCCAGGAGAGTGTCACAGAGCAGGACAGCAAGGACAGCACCTACA | |
| | | GCCTCAGCAGCACCCTGACGCTGAGCAAAGCAGACTACGAGAA | |
| S144- | HC-DNA | CAGGTTCAGCTGGTGCAGTCTGGAGCTGAGGTGAAGAAGCCTGGGGC | 1729 |
| 952 | | CTCAGTGAAGGTCTCCTGCACGGCTTCTGGTTACACCGTTACCAGTTA | |
| | | TGGTATCAGCTGGGTGCGACAGGCCCCTGGACAAGGGCTTGAGTGGA | |
| | | TGGGATGGATCAGCACTTACAATGGTAACACAAACTATGCACAGAAG | |
| | | CTCCAGGGCAGAGTCACCATGACCACAGACACATCCACGAGCACAGC | |
| | | CTACATGGAGCTGAGGAGCCTGAGATCTGACGACACGGCCGTGTATT | |
| | | ACTGTGCGAGAGAATACAGCTATGGTTACCGACTGGCCTACTTTGACT | |
| | | ACTGGGGCCAGGGAACCCTGGTCACCGTCTCCTCAGGGAGTGCATCC | |
| | | GCCCCAACCCTTTTCCCCCTCGTCTCCTGTGAGAATTCCCCGTCGGAT | |
| | | ACGAGCAGCGTGGCCGTTGGCTGCCTCGCACAGGACTTCCTTCCCGAC | |
| | | TCCATCACTTTCTCCTGGAAATACAAGAACAACTCTGACATCAGCAGC | |
| | | ACCCGGGGCTTCCCATCAGTCCTGAGAGGGGGCAAGTACGCAGCCAC | |
| | | CTCACAGGTGCTGCTGCCTTCCAAGGACGTCATG | |
| | LC-DNA | GACATCGTGATGACCCAGTCTCCAGACTCCCTGGCTGTGTCTCTGGGC | 1730 |
| | | GAGAGGGCCACCATCAACTGCAAGTCCAGCCAGAGTGTTTTAAACAG | |
| | | CTCCAACAATAAGAACTACTTAGCTTGGTACCAGCAGAAACCAGGAC | |
| | | AGCCTCCTAAGCTGCTCATTTACTGGGCATCTACCCGGGAATCCGGGG | |
| | | TCCCTGACCGATTCAGTGGCAGCGGGTCTGGGACAGATTTCACTCTCA | |
| | | CCATCAGCAGCCTGCAGGCTGAAGATGTGGCAGTTTATTACTGTCAGC | |
| | | AGTATTATAGTACTCCTCAGACGTTCGGCCAAGGGACCAAGGTGGAA | |
| | | ATCAAACGAACTGTGGCTGCACCATCTGTCTTCATCTTCCCGCCATCT | |
| | | GATGAGCAGTTGAAATCTGGAACTGCCTCTGTTGTGTGCCTGCTGAAT | |
| | | AACTTCTATCCCAGAGAGGCCAAAGTACAGTGGAAGGTGGATAACGC | |
| | | CCTCCAATCGGGTAACTCCCAGGAGAGTGTCACAGAGCAGGACAGCA | |
| | | AGGACAGCACCTACAGCCTCAGCAGCACCCTGACGCTGAGCAAAGCA | |
| | | GACTACGAGA | |
| S144- | HC-DNA | GAGGTGCAGCTGGTGGAGTCTGGGGGAGGCTTGGTCCAGCCTGGGGG | 1731 |
| 971 | | GTCCCTGAGAATCTCTTGTTCAGCCTCTGGATTCACCTTCAGTAGATA | |
| | | TGCTATGCACTGGGTCCGCCAGGCTCCAGGGAAGGGACTGGAATATG | |
| | | TTTCAGCTATTAGGAGTAATGGGGGTAGCACATACTACGCAGACTCC | |
| | | GTGAGGGGCAGATTCACCATCTCCAGAGACAATTCCAGGAACACGCT | |
| | | GTATCTTCAAATGAGCAGTCTGAGAGCTGAGGACACGGCTGTGTATT | |
| | | ACTGTGTGATAATAAACAATTTAGCAGCAGCTGGTACCCGTTTTGACT | |
| | | ACTGGGGCCAGGGAACCCTGGTCACCGTCTCCTCAGCCTCCACCAAG | |
| | | GGCCCATCGGTCTTCCCCCTGGCACCCTCCTCCAAGAGCACCTCTGGG | |
| | | GGCACAGCGGCCCTGGGCTGCCTGGTCAAGGACTACTTCCCCGAACC | |
| | | GGTGACGGTGTCGTGGAACTCAGGCGCCCTGACCAGCGGCGTGCACA | |
| | | CCTTCCCGGCTGTCCTACAGTCCTCAGGA | |
| | LC-DNA | GACATCGTGATGACCCAGTCTCCAGACTCCCTGGCTGTGTCTCTGGGC | 1732 |
| | | GAGAGGGCCACCATCAACTGCAAGTCCAGCCAGAGTGTTTTATACAG | |
| | | CTCCAACAATAAGAACTTCTTAACTTGGTACCAGCAGAAACCAGGAC | |
| | | AGCCTCCTAAGCTGCTCATTTACTGGGCATCTACCCGGGAATCCGGGG | |
| | | TCCCTGACCGATTCAGTGGCAGCGGGTCTGGGACAGATTTCACTCTCA | |
| | | CCATCAGCAGCCTGCAGGCTGAAGATGTGGCAGTTTATTACTGTCAGC | |
| | | AATATTATACTACTCCGTGGACGTTCGGCCAAGGGACCAAGGTGGAA | |
| | | ATCAAACGAACTGTGGCTGCACCATCTGTCTTCATCTTCCCGCCATCT | |
| | | GATGAGCAGTTGAAATCTGGAACTGCCTCTGTTGTGTGCCTGCTGAAT | |
| | | AACTTCTATCCCAGAGAGGCCAAAGTACAGTGGAAGGTGGATAACGC | |
| | | CCTCCAATCGGGTAACTCCCAGGAGAGTGTCACAGAGCAGGACAGCA | |
| | | AGGACAGCACCTACAGCCTCAGCAGCACCCTGACGCTGAGCAAAGCA | |
| | | GACTACGAGAA | |
| S144- | HC-DNA | CAGGTGCAGCTACAGCAGTGGGGCGCAGGGCTGTTGAAGCCTTCGGA | 1733 |
| 1036 | | GACCCTGTCCCTCACCTGCGCTGTCTATGGTGGGTCCTTCAGTGGTTA | |
| | | CTTCTGGAGCTGGATCCGCCAGCCCCCAGGGAAGGGGCTGGAGTGGA | |
| | | TTGGGGAAATCAATCATAGTGGAAGCACCAACTACAACCCGTCCCTC | |
| | | AAGAGTCGAGTCACCATATCAGTAGACACGTCCAAGAACCAGTTCTC | |
| | | CCTGAAGCTGAGCTCTGTGACCGCCGCGGACACGGCTGTGTATTACTG | |
| | | TGCGAGAGCGCCCTATTACGATTTCTTGCGGGAAGGAAACTGGTTCG | |
| | | ACCCCTGGGGCCAGGGAACCCTGGTCACCGTCTCCTCAGCCTCCACC | |
| | | AAGGGCCCATCGGTCTTCCCCCTGGCACCCTCCTCCAAGAGCACCTCT | |
| | | GGGGGCACAGCGGCCCTGGGCTGCCTGGTCAAGGACTACTTCCCCGA | |
| | | ACCGGTGACGGTGTCGTGGAACTCAGGCGCCCTGACCAGCGGCGTGC | |
| | | ACACCTTCCCGGCTGTCCTACAGTCCTCAGGACTCTACTCCCTCAGCA | |
| | | GCGTGGTGACCGTGCCCTCCAGCAGCTTGGGCACCCAGACCTACATCT | |
| | | GCAACGTGAATCACAAGCCCAGC | |
| | LC-DNA | GACATCGTGATGACCCAGTCTCCAGACTCCCTGGCTGTGTCTCTGGGC | 1734 |
| | | GAGAGGGCCACCATCAACTGCAACTCCAGCCAGAGTGTTTTATACAG | |
| | | CTCCATCAATAAGAACTACTTAGCTTGGTACCAGCAGAAACCAGCAC | |
| | | AGCCTCCTAAGGTGCTCATTTACTGGGCATCTACCCGGGAATCCGGGG | |
| | | TCCCTGACCGATTCAGTGGCAGCGGGTCTGGGACAGATTTCACTCTCA | |
| | | CCATCAGCAGCCTGCAGGCTGAAGATGTGGCAGTTTATTACTGTCAGC | |
| | | AATATTATAGGACTCCCTGGACGTTCGGCCAAGGGACCAAGGTGGAA | |
| | | ATCAAACGAACTGTGGCTGCACCATCTGTCTTCATCTTCCCGCCATCT | |
| | | GATGAGCAGTTGAAATCTGGAACTGCCTCTGTTGTGTGCCTGCTGAAT | |
| | | AACTTCTATCCCAGAGAGGCCAAAGTACAGTGGAAGGTGGATAACGC | |
| | | CCTCCAATCGGGTAACTCCCAGGAGAGTGTCACAGAGCAGGACAGCA | |
| | | AGGACAGCACCTACAGCCTCAGCAGCACCCTGACGCTGAGCAAAGCA | |
| | | GACTACGAGAA | |
| S144- | HC-DNA | CAGGTCCAGCTGGTGCAATCTGGGGCTGAGGTGAAGAAGCCTGGGTC | 1735 |
| 1079 | | CTCGGTGAAGGTCTCCTGCAAGGCTTCTGGAGACACCTTCGGCAGCTA | |
| | | TAGTATCACCTGGGTGCGACAGGCCCCTGGACAAGGACTTGAGTGGA | |
| | | TGGGAAGGATCATCCCTGTCCTTGGTATAGCAAACTACGCACAGAAG | |
| | | TTCCAGGGCAGAGTCACGATTACCGCGGACAAATCCACGAGCACAGC | |
| | | CTACATGGAGCTGAGCAGCCTGAGATCTGAGGACACGGCCGTGTATT | |
| | | ACTGTGCGGGAGGGGGTTGTAGTGGTGGTAACTGCTACTCGTGGTAC | |
| | | AACTGGTTCGACCCCTGGGGCCAGGGATCCCTGGTCACCGTCTCCTCA | |
| | | GCCTCCACCAAGGGCCCATCGGTCTTCCCCCTGGCACCCTCCTCCAAG | |
| | | AGCACCTCTGGGGGCACAGCGGCCCTGGGCTGCCTGGTCAAGGACTA | |
| | | CTTCCCCGAACCGGTGACGGTGTCGTGGAACTCAGGCGCCCTGACCA | |
| | | GCGGCGTGCACACCTTCCCGGCTGTCCTACAGTCCTCAGGA | |
| | LC-DNA | GAAATTGTGTTGACGCAGTCTCCAGGCACCCTGTCTTTGTCTCCAGGG | 1736 |
| | | GAAAGAGCCACCCTCTCCTGCAGGGCCAGTCAGAGTGTTAGCAGCAA | |
| | | CTACTTAGCCTGGTACCAGCAGAAACCTGGCCAGGCTCCCAGGCTCCT | |
| | | CATCTATGGTGCATCCAGCAGGGCCACTGGCATCCCAGAGAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTATGGTAGGTCA | |
| | | CCGTACACTTTTGGCCAGGGGACCAAGCTGGAGATCAAACGAACTGT | |
| | | GGCTGCACCATCTGTCTTCATCTTCCCGCCATCTGATGAGCAGTTGAA | |
| | | ATCTGGAACTGCCTCTGTTGTGTGCCTGCTGAATAACTTCTATCCCAG | |
| | | AGAGGCCAAAGTACAGTGGAAGGTGGATAACGCCCTCCAATCGGGTA | |
| | | ACTCCCAGGAGAGTGTCACAGAGCAGGACAGCAAGGACAGCACTTAC | |
| | | AGCCTCAGCAGCACCCTGACGCTGAGCAAAGCAGACTACGAGAA | |
| S144- | HC-DNA | CAGGTGCAGCTGCAGGAGTCGGGCCCAGGACTGGTGAAGCCTTCGGA | 1737 |
| 1299 | | GACCCTGTCCCTCACCTGCACTGTCTCTGGTGGCTCCATCAGTAGTTA | |
| | | CTACTGGAGCTGGATCCGGCAGCCCCCAGGGAAGGGACTGGAGTGGA | |
| | | TTGGGTATATCAATTACAGGGGGATCACCAACTACAACCCCTCCCTCA | |
| | | AGAGTCGAGTCACCATATCAGTAGACATGTCCAAGAACCAGTTCTCC | |
| | | CTGAAGCTGAGCTCTGTGACCGCCGCAGACACGGCCGTGTATTCCTGT | |
| | | GCGAGACTAGCAGTGGCTAGTCGAGGGACCGTTGACTACTGGGGCCA | |
| | | GGGAACCCTGGTCACCGTCTCCTCAGCCTCCACCAAGGGCCCATCGGT | |
| | | CTTCCCCCTGGCACCCTCCTCCAAGAGCACCTCTGGGGGCACAGCGG | |
| | | CCCTGGGCTGCCTGGTCAAGGACTACTTCCCCGAACCGGTGACGGTGT | |
| | | CGTGGAACTCAGGCGCTCTGACCAGCGGCGTGCACACCTTCCCAGCT | |
| | | GTCCTACAGTCCTCAGGACTCTACTCCCTCAGCAGCGTGGTGACCGTG | |
| | | CCCTCCAGCAACTTCGGCACCCAGACCTACACCTGCAACGTAGATCA | |
| | | CAAGCCCAGCAACACCAAGGTGGAC | |
| | LC-DNA | CAGTCTGTGCTGACTCAGCCACCCTCAGCGTCTGGGACCCCCGGGCA | 1738 |
| | | GAGGGTCACCATCTCTTGTTCTGGAAGCAGCTCCAACATCGGAAGTA | |
| | | ATTATGTATACTGGTACCAGCAGCTCCCAGGAACGGCCCCCAAACTC | |
| | | CTCATCTATAGGAATAATCAGCGGCCCTCAGGGGTCCCTGACCGATTC | |
| | | TCTGGCTCCAAGTCTGGCACCTCAGCCTCCCTGGCCATCAGTGGGCTC | |
| | | CGGTCCGAGGATGAGGCTGATTATTACTGTGCAGCATGGGATGACAG | |
| | | CCTGAGTGTTAATGTGGTATTCGGCGGAGGGACCAAGCTGACCGTCC | |
| | | TAGGTCAGCCCAAGGCTGCCCCCTCGGTCACTCTGTTCCCGCCCTCCT | |
| | | CTGAGGAGCTTCAAGCCAACAAGGCCACACTGGTGTGTCTCATAAGT | |
| | | GACTTCTACCCGGGAGCCGTGACAGTGGCCTGGAAGGCAGATAGCAG | |
| | | CCCCGTCAAGGCGGGAGTGGAGACCACCAAACCCTCCAAACAGAGCA | |
| | | ACAACAAGTACGCGGCCAGCAGCTACCTGAGCCTGACGCCTGAGCAG | |
| | | TGGAAGTCCCACA | |
| S144- | HC-DNA | CAGGTGCAGCTGGTGCAGTCTGGGACTGAGGTGAAGAAGCCTGGGGC | 1739 |
| 1339 | | CTCAGTGAAGGTCTCCTGCAAGGCTTCTGGATACACCTTCACCGACTA | |
| | | CTATATGCACTGGGTGCGACAGGCCCCTGGACAAGGGCTTGAGTGGA | |
| | | TGGGACGGATCAACCCTACCAGTGGTGGCACAAACTATCCACAGAAG | |
| | | TTTCAGGGCAGTGTCACCATGACCAGGGACACGTCCCTCAGCACAGT | |
| | | CTACATGGAACTGAGCGGGCTGAGATCTGACGACACGGCCGTCTATT | |
| | | ATTGTGCGAGAGAGAGGGTTACTCTGATTCAGGGAAAGAACCACTAC | |
| | | TACATGGACGTCTGGGGCACAGGGACCACGGTCACCGTCTCCTCAGC | |
| | | CTCCACCAAGGGCCCATCGGTCTTCCCCCTGGCACCCTCCTCCAAGAG | |
| | | CACCTCTGGGGGCACAGCGGCCCTGGGCTGCCTGGTCAAGGACTACT | |
| | | TCCCCGAACCGGTGACGGTGTCGTGGAACTCAGGCGCCCTGACCAGC | |
| | | GGCGTGCACACCTTCCCGGCTGTCCTACAGTCCTCAGGA | |
| | LC-DNA | CAGTCTGCCCTGACTCAGCCTGCCTCCGTGTCTGGGTCTCCTGGACAG | 1740 |
| | | TCGATCACCATCTCCTGCACTGGAACCAACAGTGACGTTGGTGGTTAT | |
| | | AACTATGTCTCCTGGTACCAACAACACCCAGGCAAAGCCCCCAGACT | |
| | | CATGATTTATGATGTCAGTAATCGGCCCTCAGGGGTTTCTAATCGCTT | |
| | | CTCTGGCTCCAAGTCTGGCAACACGGCCTCCCTGACCATCTCTGGGCT | |
| | | CCAGGCTGAGGACGAGGCTGATTATTACTGCAGCTCATATACAAGCA | |
| | | GCAGCACTCTCGTGGTTTTCGGCGGAGGGACCAAGCTGACCGTCCTA | |
| | | GGTCAGCCCAAGGCTGCCCCCTCGGTCACTCTGTTCCCGCCCTCCTCT | |
| | | GAGGAGCTTCAAGCCAACAAGGCCACACTGGTGTGTCTCATAAGTGA | |
| | | CTTCTACCCGGGAGCCGTGACAGTGGCCTGGAAGGCAGATAGCAGCC | |
| | | CCGTCAAGGCGGGAGTGGAGACCACCACACCCTCCAAACAAAGCAAC | |
| | | AACAAGTACGCGGCCAGCAGCTATCTGAGCCTGACGCCTGAGCAGTG | |
| | | GAAGTCCCACA | |
| S144- | HC-DNA | CAGGTCCAGCTTGTGCAGTCTGGGGCTGAGGTGAAGAAGCCTGGGGC | 1741 |
| 1406 | | CTCAGTGAAGGTTTCCTGCAAGGCTTCTGGATATACCTTCACTACCTA | |
| | | TGCTATGCATTGGGTGCGCCAGGCCCCCGGACAAAGGCTTGAGTGGA | |
| | | TGGGATGGATCAACGCTGGCAATGGTAACACAAAATATTCACAGAAC | |
| | | TTCCAGGGCAGAGTCACCATTACCAGGGACACATCCGCGAGCACAGC | |
| | | CTACATGGAGCTGAGCAGCCTGAGATCTGAAGACACGGCTGTGTATT | |
| | | ACTGTGCGAGTCTCGTGGGTGGGGATAGCAGCAGCTGGTATGACTAC | |
| | | ATGGACGTCTGGGGCAAAGGGACCACGGTCACCGTCTCCTCAGCCTC | |
| | | CACCAAGGGCCCATCGGTCTTCCCCCTGGCGCCCTGCTCCAGGAGCA | |
| | | CCTCCGAGAGCACAGCGGCCCTGGGCTGCCTGGTCAAGGACTACTTC | |
| | | CCCGAACCGGTGACGGTGTCGTGGAACTCAGGCGCTCTGACCAGCGG | |
| | | CGTGCACACCTTCCCAGCTGTCCTACAGTCCTCAGGACTCTACTCCCT | |
| | | CAGCAGCGTGGTGACCGTGCCCTCCAGCAACTTCGG | |
| | LC-DNA | GACATCCAGATGACCCAGTCTCCTTCCACCCTGTCTGCATCTGTAGGA | 1742 |
| | | GACAGAGTCACCATCACTTGCCGGGCCAGTCAGAGTATTAGTAGCTG | |
| | | GTTGGCCTGGTATCAGCAGAAACCAGGGAAAGCCCCTAAGCTCCTGA | |
| | | TCTATGATGCCTCCAGTTTGGAAAGTGGGGTCCCATCAAGGTTCAGCG | |
| | | GCAGTGGATCTGGGACAGAATTCACTCTCACCATCAGCAGCCTGCAG | |
| | | CCTGATGATTTTGCAACTTATTACTGCCAACAGTATAATAGTTATCCG | |
| | | TGGACGTTCGGCCAAGGGACCAAGGTGGAAATCAAACGAACTGTGGC | |
| | | TGCACCATCTGTCTTCATCTTCCCGCCATCTGATGAGCAGTTGAAATC | |
| | | TGGAACTGCCTCTGTTGTGTGCCTGCTGAATAACTTCTATCCCAGAGA | |
| | | GGCCAAAGTACAGTGGAAGGTGGATAACGCCCTCCAATCGGGTAACT | |
| | | CCCAGGAGAGTGTCACAGAGCAGGACAGCAAGGACAGCACCTACAG | |
| | | CCTCAGCAGCACCCTGACGCTGAGCAAAGCAGACTACGAGAA | |
| S144- | HC-DNA | CAGGTCCAGCTGGTGCAATCTGGGGCTGAGGTGAAGAAGCCTGGGTC | 1743 |
| 1407 | | CTCGGTGAAGGTCTCCTGCAAGGCTTCTGGAGGCACCTTCAGCAGCTA | |
| | | TACTATCAGCTGGGTGCGACAGGCCCCTGGACAAGGCCTTGAGTGGA | |
| | | TGGGAAGGATCATCCCTGTCCGTGATATAGCAAACTACGCACAGAAG | |
| | | TTCCAGGGCAGAGTCACGATTACCGCGGACAAATCCACGAGGACAGC | |
| | | CTACATGGAGGTGAGCAGCCTGAGATCTGAGGACACGGCCGTGTATT | |
| | | ACTGTGCGGCAACGGAGCTCCGCTCGGATGGTCTTGACATCTGGGGC | |
| | | CAAGGGACAATGGTCACCGTCTCTTCAGCCTCCACCAAGGGCCCATC | |
| | | GGTCTTCCCCCTGGCACCCTCCTCCAAGAGCACCTCTGGGGGCACAGC | |
| | | GGCCCTGGGCTGCCTGGTCAAGGACTACTTCCCCGAACCGGTGACGG | |
| | | TGTCGTGGAACTCAGGCGCCCTGACCAGCGGCGTGCACACCTTCCCG | |
| | | GCTGTCCTACAGTCCTCAGGA | |
| | LC-DNA | GACATCCAGATGACCCAGTCTCCTTCCACCCTGTCTGCATCTGTAGGA | 1744 |
| | | GACAGAGTCACCATCACTTGCCGGGCCAGTCAGAGTATTAGTAGCTG | |
| | | GTTGGCCTGGTATCAGCAGAAACCAGGGAAAGCCCCTAAGCTCCTGA | |
| | | TCTATGATGCCTCCAGTTTGGAAAGTGGGGTCCCATCAAGGTTCAGCG | |
| | | GCAGTGGATCTGGGACAGAATTCACTCTCACCGTCAGCAGCCTGCAG | |
| | | CCTGATGATTTTGCAACTTATTACTGCCAACAGTATAATAATTATTCT | |
| | | CCCATCACTTTTGGCCAGGGGACCAAGCTGGAGATCAAACGAACTGT | |
| | | GGCTGCACCATCTGTCTTCATCTTCCCGCCATCTGATGAGCAGTTGAA | |
| | | ATCTGGAACTGCCTCTGTTGTGTGCCTGCTGAATAACTTCTATCCCAG | |
| | | AGAGGCCAAAGTACAGTGGAAGGTGGATAACGCCCTCCAATCGGGTA | |
| | | ACTCCCAGGAGAGTGTCACAGAGCAGGACAGCAAGGACAGCACCTA | |
| | | CAGCCTCAGCAGCACCCTGACGCTGAGCAAAGCAGACTACGAGAA | |
| S144- | HC-DNA | CAGGTTCAGCTGGTGCAGTCTGGAGCTGAGGTGAAGAAGCCTGGGGC | 1745 |
| 1569 | | CTCAGTGAAGGTCTCCTGCAAGGCTTCTGGTTACACCTTTTCCAACTA | |
| | | CGGTATCAGCTGGGTGCGACAGGCCCCTGGACAAGGGCTTGAGTGGA | |
| | | TGGGATGGATCAGCGCTTACAATGGTAACACTAAGTATCCACAAAAG | |
| | | CTCCAGGGCAGAGTCACCATGAGCACAGACACATCCACGAGCACAGC | |
| | | CTACATGGAGCTGAGGAGCCTGAGATCTGACGACACGGCCGTGTATT | |
| | | ACTGTGCGAGAGAGACGCGGTACGGTATGGACGTCTGGGGCCAAGGG | |
| | | ACCACGGTCACCGTCTCCTCAGCCTCCACCAAGGGCCCATCGGTCTTC | |
| | | CCCCTGGCACCCTCCTCCAAGAGCACCTCTGGGGGCACAGCGGCCCT | |
| | | GGGCTGCCTGGTCAAGGACTACTTCCCCGAACCGGTGACGGTGTCGT | |
| | | GGAACTCAGGCGCCCTGACCAGCGGCGTGCACACCTTCCCGGCTGTC | |
| | | CTACAGTCCTCAGGA | |
| | LC-DNA | CAGCCTGTGCTGACTCAGCCACCTTCTGCATCAGCCTCCCTGGGAGCC | 1746 |
| | | TCGGTCACACTCACCTGCACCCTGAGCAGCGGCTACAGTAATTATAA | |
| | | AGTGGACTGGTACCAGCAGAGACCAGGGAAGGGCCCCCAGTTTGTGA | |
| | | TGCGAGTGGGCACTGGTGGGATTGTGGGATCCAAGGGGGATGGCATC | |
| | | CCTGATCGCTTCTCAGTCTTGGGCTCAGGCCTGAATCGGTACCTGACC | |
| | | ATCAAGAACATCCAGGAAGAGGATGAGAGTGACTACCACTGTGGGGC | |
| | | AGACCATGGCAGTGGGAGCAACTTCGTTCGGGTGTTCGGCGGAGGGA | |
| | | CCAAGCTGACCGTCCTAGGTCAGCCCAAGGCTGCCCCCTCGGTCACTC | |
| | | TGTTCCCACCCTCCTCTGAGGAGCTTCAAGCCAACAAGGCCACACTGG | |
| | | TGTGTCTCATAAGTGACTTCTACCCGGGAGCCGTGACAGTGGCCTGGA | |
| | | AGGCAGATAGCAGCCCCGTCAAGGCGGGAGTGGAGACCACCACACC | |
| | | CTCCAAACAAAGCAACAACAAGTACGCGGCCAGCAGCTACCTGAGCC | |
| | | TGACGCCTGAGCAGTGGAAGTCCCAC | |
| S144- | HC-DNA | GAGGTGCAGCTGGTGCAGTCTGGAGCAGAGGTGAAAAAGCCCGGGG | 1747 |
| 1641 | | AGTCTCTGAAGATCTCCTGTAAGGGTTCTGGATACACCTTTACCAGCT | |
| | | ACTGGATCGGCTGGGTGCGCCAGATGCCCGGGAAAGGCCTGGAGTGG | |
| | | ATGGGGATCATCTATCTTGGTGACTCTGATACGAGATACAGCCCGTCC | |
| | | TTCCAAGGCCAGGTCACCATCTCAGCCGACAAGTCCATCAGCACCGC | |
| | | CTACCTGCAGTGGAACAGCCTGAAGGCCTCGGACACCGCCATGTATT | |
| | | ACTGTGCGAGACAGGTTACCGGAACTACGAGCTGGTTCGACCCCTGG | |
| | | GGCCAGGGAACCCTGGTCACCGTCTCCTCAGCCTCCACCAAGGGCCC | |
| | | ATCGGTCTTCCCCCTGGCACCCTCCTCCAAGAGCACCTCTGGGGGCAC | |
| | | AGCGGCCCTGGGCTGCCTGGTCAAGGACTACTTCCCCGAACCGGTGA | |
| | | CGGTGTCGTGGAACTCAGGCGCCCTGACCAGCGGCGTGCACACCTTC | |
| | | CCGGCTGTCCTACAGTCCTCAGGA | |
| | LC-DNA | GACATCCAGATGACCCAGTCTCCTTCCACCCTGTCTGCATCTGTAGGA | 1748 |
| | | GAGAGAGTCACCATCACTTGCCGGGCCAGTCAGAGTATTAGTAGGTG | |
| | | GTTGGCCTGGTATCAGCAGAAACCAGGGAAAGCCCCTAAACTCCTTA | |
| | | TCTATGATGCCTCCAGTTTGGAAAGTGGGGTCCCATCAAGGTTCAGCG | |
| | | GCAGTGGATCTGGGACAGAATTCACTCTCACCATCAGCAGCCTGCAG | |
| | | CCTGATGATTTTGCAACTTATCACTGCCACCAGTATAGTACTTATTCG | |
| | | CTCACTTTCGGCGGAGGGACCAAGGTGGACATCAAACGAACTGTGGC | |
| | | TGCACCATCTGTCTTCATCTTCCCGCCATCTGATGAGCAGTTGAAATC | |
| | | TGGAACTGCCTCTGTTGTGTGCCTGCTGAATAACTTCTATCCCAGAGA | |
| | | GGCCAAAGTACAGTGGAAGGTGGATAACGCCCTCCAATCGGGTAACT | |
| | | CCCAGGAGAGTGTCACAGAGCAGGACAGCAAGGACAGCACCTACAG | |
| | | CCTCAGCAGCACCCTGACGCTGAGCAAAGCAGACTACGAGAA | |
| S144- | HC-DNA | GAGGTGCAGCTGGTGGAGTCTGGGGGAGACGTGGTCCAGCCTGGGGG | 1749 |
| 1827 | | GTCCCTGAGACTCTCCTGTGCAGCCTCTGGAATTACCTTTAGTAACTA | |
| | | TTGGATGACCTGGGTCCGCCAGGCTCCAGGGAAAGGGCTGGAGTGGG | |
| | | TGGCCACCATAAAGAAGGATGGAGGGGAGCAGTACTATGTGGACTCT | |
| | | GTGAAGGGCCGATTCACCATCTCCAGAGACAACGCCAGGAATTCACT | |
| | | GTATCTACAAATAAACAGCCTGAGGGCCGAGGATACGGCTGTCTATT | |
| | | ACTGTGCGAGGGGTGGATCTAGCAGCAGCTACTACTGGATCTACTGG | |
| | | GGCCAGGGAACCCTGGTCACCGTCTCCTCAGGGAGTGCATCCGCCCC | |
| | | AACCCTTTTCCCCCTCGTCTCCTGTGAGAATTCCCCGTCGGATACGAG | |
| | | CAGCGTG | |
| | LC-DNA | GAAATTGTGTTGACGCAGTCTCCAGGCACCCTGTCTTTGTCTCCAGGG | 1750 |
| | | GAAAGAGCCACCCTCTCCTGCAGGGCCAGTCAGAGTATTAGCAACAG | |
| | | CTACTTAGTCTGGTACCAGCAGAAACCTGGCCAGGCTCCCAGGCTCCT | |
| | | CATCTATGGTGCATCCACCAGGGCCACTGGCATCCCAGACAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTATGGTAGCTCA | |
| | | CCGTGGACGTTCGGCCAAGGGACCACGGTGGAAATCAAACGAACTGT | |
| | | GGCTGCACCATCTGTCTTCATCTTCCCGCCATCTGATGAGCAGTTGAA | |
| | | ATCTGGAACTGCCTCTGTTGTGTGCCTGCTGAATAACTTCTATCCCAG | |
| | | AGAGGCCAAAGTACAGTGGAAGGTGGATAACGCCCTCCAATCGGGTA | |
| | | ACTCCCAGGAGAGTGTCACAGAGCAGGACAGCAAGGACAGCACCTA | |
| | | CAGCCTCAGCAGCACCCTGACGCTGAGCAAAGCAGACTACGAGAA | |
| S144- | HC-DNA | GAGGTGCAGCTGGTGGAGTCTGGGGGAGGCCTGGTCAAGCCTGGGGG | 1751 |
| 1848 | | GTCCCTGAGACTCTCCTGTGCAGCCTCTGGATTCACCTTCAGTAGCTA | |
| | | TAGCATGAACTGGGTCCGCCAGGCTCCAGGGAAGGGGCTGGAGTGGG | |
| | | TCTCGTCCATTAGTAGTAGTAGTAGTTACATATACTACGCAGACTCAG | |
| | | TGAAGGGCCGATTCACCATCTCCAGAGACAACGCCAAGAATTCACTG | |
| | | TATCTGCAACTGAACAGCCTGAGAGCCGAGGACACGGCTGTGTACTA | |
| | | CTGTGCGAGAGATCGGGACCAGTTGATATTCTCGGCCGCTTTTGATAT | |
| | | CTGGGGCCAAGGGACAATGGTCACCGTCTCTTCAGCCTCCACCAAGG | |
| | | GCCCATCGGTCTTCCCCCTGGCACCCTCCTCCAAGAGCACCTCTGGGG | |
| | | GCACAGCGGCCCTGGGCTGCCTGGTCAAGGACTACTTCCCCGAACCG | |
| | | GTGACGGTGTCGTGGAACTCAGGCGCCCTGACCAGCGGCGTGCACAC | |
| | | CTTCCCGGCTGTCCTACAGTCCTCAGGA | |
| | LC-DNA | CAGTCTGTGCTGACTCAGCCACCCTCAGCGTCTGGGACCCCCGGGCA | 1752 |
| | | GAGGGTCACCATCTCTTGTTCTGGAAGCAGCTCCAACATCGAACATA | |
| | | ATTATGTATTCTGGTACCAGCAACTCCCAGGAACGGCCCCCAAACTCC | |
| | | TCATCTATAGTAATAATCACCGGCCCTCAGGGGTCCCTGACCGATTCT | |
| | | CTGGCTCCAAGTCTGGCACCTCAGCCTCCCTGGCCATCAGTGGGCTCC | |
| | | GGTCCGAGGATGAGGCTGATTATTACTGTGCAGCATGGGATGCCAGC | |
| | | CTGAGTGGTCCTGTGGTATTCGCCGGAGGGACCAAGCTGACCGTCCT | |
| | | AGGTCAGCCCAAGGCTGCCCCCTCGGTCACTCTGTTCCCGCCCTCCTC | |
| | | TGAGGAGCTTCAAGCCAACAAGGCCACACTGGTGTGTCTCATAAGTG | |
| | | ACTTCTACCCGGGAGCCGTGACAGTGGCCTGGAAGGCAGATAGCAGC | |
| | | CCCGTCAAGGCGGGAGTGGAGACCACCACACCCTCCAAACAAAGCAA | |
| | | CAACAAGTACGCGGCCAGCAGCTA | |
| S144- | HC-DNA | GAGGTGCAGCTGGTGGAGTCTGGGGGAGGCTTGGTACAGCCTGGGGG | 1753 |
| 1850 | | GTCCCTGAGACTCTCCTGTGCAGCCTCTGGATTCACCTTTAGCAGCTA | |
| | | TGCCATGAGTTGGGTCCGCCAGGCTCCAGGGAAGGGGCTGGAGTGGG | |
| | | TCTCAGCTATTAGTGGTAGTGGTGGTAGCACATACTACGCAGACTCCG | |
| | | TGAAGGGCCGGTTCACCATCTCCAGAGCCAATTCCAAGAACACGCTG | |
| | | TATCTGCAAATGAACAGCCTGAGAGCCGAGGACACGGCCGTATATTA | |
| | | CTGTGCGAAAGGCCCGCGCTTTAGTCGCGACTACTTTGACTACTGGGG | |
| | | CCAGGGAACCCTGGTCACCGTCTCCTCAGCCTCCACCAAGGGCCCAT | |
| | | CGGTCTTCCCCCTGGCACCCTCCTCCAAGAGCACCTCTGGGGGCACAG | |
| | | CGGCCCTGGGCTGCCTGGTCAAGGACTACTTCCCCGAACCGGTGACG | |
| | | GTGTCGTGGAACTCAGGCGCCCTGACCAGCGGCGTGCACACCTTCCC | |
| | | GGCTGTCCTACAGTCCTCAGGA | |
| | LC-DNA | GACATCCAGATGACCCAGTCTCCTTCCACCCTGTCTGCATCTGTAGGA | 1754 |
| | | GACAGAGTCACCATCACTTGCCGGGCCAGTCAGAGTATTACTAGCTG | |
| | | GTTGGCCTGGTATCAGCAGAAACCAGGGAAAGCCCCTAAGCTCCTGA | |
| | | TCTATGATGCCTCCAATTTGGAAAGTGGGGTCCCATCAAGGTTCAGCG | |
| | | GCAGTGGATCTGGGACAGAATTCACTCTCACCATCAGCAGCCTGCAG | |
| | | CCTGATGATTTTGCAACTTATTACTGCCAACAGTATAATAATTATCTG | |
| | | GGGACGTTCGGCCAAGGGACCAAGGTGGAAATCAAACGAACTGTGG | |
| | | CTGCACCATCTGTCTTCATCTTCCCGCCATCTGATGAGCAGTTGAAAT | |
| | | CTGGAACTGCCTCTGTTGTGTGCCTGCTGAATAACTTCTATCCCAGAG | |
| | | AGGCCAAAGTACAGTGGAAGGTGGATAACGCCCTCCAATCGGGTAAC | |
| | | TCCCAGGAGAGTGTCACAGAGCAGGACAGCAAGGACAGCACCTACA | |
| | | GCCTCAGCAGCACCCTGACGCTGAGCAAAGCAGACTACGAGAA | |
| S144- | HC-DNA | CAGGTCCAGCTGGTGCAATCTGGGGCTGAGGTGAAGAAGCCTGGGTC | 1755 |
| 2234 | | CTCGGTGAAGGTCTCCTGCAAGGCTTCTGGAGGCACCTTCAGCAGAT | |
| | | ATACTATCAGCTGGGTGCGACAGGCCCCTGGACAAGGGCTTGAGTGG | |
| | | ATGGGAAGGATCATCCCTATACTTGGTACAGCAAACTACGCACAGAA | |
| | | TTTCCAGGGCAGAGTCACGATTACCGCGGACAAATCCACGAGCACAG | |
| | | CCTACATGGAGCTGAGTAGCCTGAGATCTGAGGACACGGCCGTGTAT | |
| | | TACTGTGCGAGACACGGATACAGCTATGGTCCCTTTGACTACTGGGGC | |
| | | CAGGGAACCCTGGTCACCGTCTCCTCAGCCTCCACCAAGGGCCCATC | |
| | | GGTCTTCCCCCTGGCACCCTCCTCCAAGAGCACCTCTGGGGGCACAGC | |
| | | GGCCCTGGGCTGCCTGGTCAAGGACTACTTCCCCGAACCGGTGACGG | |
| | | TGTCGTGGAACTCAGGCGCCCTGACCAGCGGCGTGCACACCTTCCCG | |
| | | GCTGTCCTACAGTCCTCAGGAG | |
| | LC-DNA | GACATCGTGATGACCCAGTCTCCAGACTCCCTGACTGTGTCTCTGGGC | 1756 |
| | | GAGAGGGCCACCATCAACTGCAAGTCCAGCCAGAGTGTTTTATACAG | |
| | | CTCCAACAATAAGAACTACTTAGCTTGGTACCAGCAGAAACCAGGAC | |
| | | AGCCTCCTAAGCTGCTCATTTACTGGGCATCTACCCGGGAATCCGGGG | |
| | | TCCCTGACCGATTCAGTGGCAGCGGCTCTGGGACAGATTTCACTCTCA | |
| | | CCGTCAGCAGCCTGCAGGCTGAAGATGTGGCAGTTTATTACTGTCAGC | |
| | | AATATTATAGTACTCCTGGAACGTTCGGCCAAGGGACCAAGGTGGAA | |
| | | ATCAAACGAACTGTGGCTGCACCATCTGTCTTCATCTTCCCGCCATCT | |
| | | GATGAGCAGTTGAAATCTGGAACTGCCTCTGTTGTGTGCCTGCTGAAT | |
| | | AACTTCTATCCCAGAGAGGCCAAAGTACAGTGGAAGGTGGATAACGC | |
| | | CCTCCAATCGGGTAACTCCCAGGAGAGTGTCACAGAGCAGGACAGCA | |
| | | AGGACAGCACCTACAGCCTCAGCAGCACCCTGACGCTGAGCAAAGCA | |
| | | GACTACGAGAA | |
| S564- | HC-DNA | CAGGTGCGGCTGCAGGAGTCGGGCCCAGGACTGGTGAAGCCTTCACA | 1757 |
| 105 | | GACCCTGTCCCTCACCTGCACTGTCTCTGGTGGCTCCATCAGCAGTGG | |
| | | TAGTTACTACTGGAGCTGGATCCGGCAGCCCGCCGGGAAGGGACTGG | |
| | | AGTGGATTGGGCGTTTCCATACCAGTGGGAGCACCAACTACAATCCC | |
| | | TCCCTCAAGAGTCGAGTCACCATATCAGTAGACACGTCCAAGAACCA | |
| | | GTTCTCCCTGAAGCTGAGTTCTGTGACCGCCGCAGACACGGCCGTGTA | |
| | | TTACTGTGCGAGAGATTTAAAGGGAAAGACGTGGATACAGACCCCCT | |
| | | TTGACTACTGGGGCCAGGGAATCCTGGTCACCGTCTCCTCAGCCTCCA | |
| | | CCAAGGGCCCATCTGTCTTCCCCCTGGCACCCTCCTCCAAGAGCACCT | |
| | | CTGGGGGCACAGCGGCCCTGGGCTGCCTGGTCAAGGACTACTTCCCC | |
| | | GAACCGGTGACGGTGTCGTGGAACTCAGGCGCTCTGACCAGCGGCGT | |
| | | GCACACCTTCCCGGCTGTCCTACAGTCCTCAGGA | |
| | LC-DNA | CAGTCTGCCCTGACTCAGCCTGCCTCCGTGTCTGGGTCTCCTGGACAG | 1758 |
| | | TCGATCACCATCTCCTGCACTGGAACCAGCAGTGACGTTGGTGCTTAT | |
| | | AACTATGTCTCCTGGTACCAACAGCACCCAGGCAAAGCCCCCAAACT | |
| | | CATGATTTATGAGGTCAGTAATCGGCCCTCAGGGGTTTCTAATCGCTT | |
| | | CTCTGGCTCCAAGTCTGGCAACACGGCCTCCCTGACCATCTCTGGGCT | |
| | | CCAGGCTGAGGACGAGGCTGATTATTACTGCAGCTCATATACAAGCA | |
| | | GCACCTTCTTCGGAACTGGGACCACGGTCACCGTCCTAGGTCAGCCCA | |
| | | AGGCCAACCCCACTGTCACTCTGTTCCCGCCCTCCTCTGAGGAGCTCC | |
| | | AAGCCAACAAGGCCACACTAGTGTGTCTGATCAGTGACTTCTACCCG | |
| | | GGAGCTGTGACAGTGGCCTGGAAGGCAGATGGCAGCCCCGTCAAGGC | |
| | | GGGAGTGGAGACCACCACACCCTCCAAACAAAGCAACAACAAGTAC | |
| | | GCGGCCAGCAGCTAC | |
| S564- | HC-DNA | GAGGTGCAGCTGGTGGAGTCTGGGGGAGGCTTGGTCCAGCCTGGGGG | 1759 |
| 14 | | GTCCCTGAGACTCTCCTGTGCAGCCTCTGGACTCACCTTTAGTAGCTA | |
| | | TTGGATGAGCTGGGCCCGCCAGGCTCCAGGGAAGGGGCTGGAGTGGG | |
| | | TGGCCAATATAAAGAAAGATGGAAGTGAGAAATACTATGTGGACTCT | |
| | | GTGAAGGGCCGATTCACCATCTCCAGAGACAACGCCAAGAACTCACT | |
| | | GTATCTGCAAATGAACAGCCTGAGAGTCGAGGACACGGCTGTGTATT | |
| | | ACTGTGCGAGTGAACCTCCCCACTACGGTGGTAACTCCGGGGCTGAA | |
| | | TACTTCCAGCACTGGGGCCAGGGCACCCTGGTCACCGTCTCCTCAGCA | |
| | | CCCACCAAGGCTCCGGATGTGTTCCCCATCATATCAGGGTGCAGACA | |
| | | CCCAAAGGATAACAGCCCTGTGGTCCTGGCATGCTTGATAACTGGGT | |
| | | ACCACCC | |
| | LC-DNA | TCCTATGTGCTGACTCAGCCACCCTCAGTGTCAGTGGCCCCAGGAAAG | 1760 |
| | | ACGGCCAGGATTACCTGTGGGGGAAACAACATTGGAAGTAAAAGTGT | |
| | | GCACTGGTACCAGCAGAGGCCAGGCCAGGCCCCTGTACTGGTCATCT | |
| | | ATTATGATAGCGACCGGCCCTCAGGGATCCCTGAGCGATTCTCTGGCT | |
| | | CCAACTCTGGGAACACGGCCACCCTGACCATCAGCAGGGTCGAGGCC | |
| | | GGGGATGAGGCCGACTATTACTGTCAGGTGTGGGATAGTAGTAGTGA | |
| | | TCACCATTATGTCTTCGGAACTGGGACCAAGGTCACCGTCCTAGGTCA | |
| | | GCCCAAGGCCAACCCCACTGTCACTCTGTTCCCGCCCTCCTCTGAGGA | |
| | | GCTTCAAGCCAACAAGGCCACACTGGTGTGTCTCATAAGTGACTTCTA | |
| | | CCCGGGAGCCGTGACAGTGGCCTGGAAGGCAGATAGCAGCCCCGTCA | |
| | | AGGCGGGAGTGGAGACCACCAAACCCTCCAAACAGAGCAACAACAA | |
| | | GTACGCGGCCAGCAGCTA | |
| S564- | HC-DNA | CAGGTGCAGCTGGTGCAGTCTGGGGCTGAGGTGAAGAAGCCTGGGGC | 1761 |
| 68 | | CTCAGTGAAGGTCTCCTGCAAGGCTTCTGGATACATCTTCACCGGCTA | |
| | | TTATATGCACTGGGTGCGACAGGCCCCTGGACAAGGGCTTGAGTGGA | |
| | | TGGGATGGATCAACCCTAACAGTGGTGGCACTAACTATGCACAGAAG | |
| | | TTTCAGGGCAGGGTCACCATGACCAGGGACACGTCCATCACCACAGC | |
| | | CTACATGGAGCTGAGCAGGCTGAGATCTGACGACACGGCCTTTTATT | |
| | | ACTGTGCGAGAGTCAAGAGGTTTTCGATTTTTGGAGTGGAGCTTGACT | |
| | | ACTGGGGCCAGGGAACCCTGGTCACCGTCTCCTCAGCCTCCACCAAG | |
| | | GGCCCATCGGTCTTCCCCCTGGCACCCTCCTCCAAGAGCACCTCTGGG | |
| | | GGCACAGCGGCCCTGGGCTGCCTGGTCAAGGACTACTTCCCCGAACC | |
| | | GGTGACGGTGTCGTGGAACTCAGGCGCCCTGACCAGCGGCGTGCACA | |
| | | CCTTCCCGGCTGTCCTACAGTCCTCAGGA | |
| | LC-DNA | CAGTCTGCCCTGACTCAGCCTCCCTCCGCGTCCGGGTCTCCTGGACAG | 1762 |
| | | TCAGTCACCATCTCCTGCACTGGAACCAGCAGTGACGTTGGTGGTTAT | |
| | | AACTATGTCTCCTGGTACCAACAGCACCCAGGCAAAGCCCCCAAACT | |
| | | CATGATTTATGAGGTCAGTAAGCGGCCCTCAGGGGTCCCTGATCGCTT | |
| | | CTCTGGCTCCAAGTCTGGCAACACGGCCTCCCTGACCGTCTCTGGGCT | |
| | | CCAGGCTGAGGATGAGGCTGATTATTTCTGCAGCTCATATGCAGACA | |
| | | GCAACAATTTGGTATTCGGCGGAGGGACCAAGCTGACCGTCCTAGGT | |
| | | CAGCCCAAGGCTGCCCCCTCGGTCACTCTGTTCCCGCCCTCCTCTGAG | |
| | | GAGCTTCAAGCCAACAAGGCCACACTGGTGTGTCTCATAAGTGACTT | |
| | | CTGCCCGGGAGCCGTGACAGTGGCCTGGAAGGCAGATAGCAGCCCCG | |
| | | TCAAGGCGGGAGTGGAGACCACCACACCCTCCAAACAAAGCAACAA | |
| | | CAAGTACGCGGCCAGCAGCTACC | |
| S564- | HC-DNA | CAGGTGCAGCTGCAGGAGTCGGGCCCAGGACTGGTGAAGCCTTCGGA | 1763 |
| 98 | | GACCCTGTCCCTCACCTGCACTGTCTCTGGTGGCTCCATCAGTAGTTA | |
| | | CTACTGGAGCTGGATCCGGCAGCCCCCAGGGAAGGGACTGGAGTGGA | |
| | | TTGGGTATATCTATTACAGTGGGAGCACCAACTACAACCCCTCCCTCA | |
| | | AGAGTCGAGTCACCATATCAGTAGACACGTCCAAGAACCAGTTCTCC | |
| | | CTGAAGCTGAGCTCTGTGACCGCCGCAGACACGGCCGTGTATTACTGT | |
| | | GCGAGACATCAATCGCGGTGGAATATAGTGGCTACGATGGACTTTGA | |
| | | CTACTGGGGCCAGGGAACCCTGGTCACCGTCTCCTCAGCCTCCACCAA | |
| | | GGGCCCATCGGTCTTCCCCCTGG | |
| | LC-DNA | GACATCCAGATGACCCAGTCTCCATCCTCCCTGTCTGCATCTGTAGGA | 1764 |
| | | GACAGAGTCACCATCACTTGCCGGGCAAGTCAGAGCATTCGCAGCTA | |
| | | TTTAAATTGGTATCAGCAGAAACCAGGGAAAGCCCCTAAGCTCCTGA | |
| | | TCTATGCTGCATCCAGTTTGCAAAGTGGCGTCCCATCAAGGTTCAGTG | |
| | | GCAGTGGATCTGGGACAGATTTCACTCTCACCATCGGCAGTCTGCAAC | |
| | | CTGAAGATTTTGCAACTTACTACTGTCAACAGAGTTACAGTACCTCCG | |
| | | TGGCGTTCGGCCAAGGGACCAAGGTGGAAATCAAACGAACTGTGGCT | |
| | | GCACCATCTGTCTTCATCTTCCCGCCATCTGATGAGCAGTTGAAATCT | |
| | | GGAACTGCCTCTGTTGTGTGCCTGCTGAATAACTTCTATCCCAGAGAG | |
| | | GCCAAAGTACAGTGGAAGGTGGATAACGCCCTCCAATCGGGTAACTC | |
| | | CCAGGAGAGTGTCACAGAGCAGGACAGCAAGGACAGCACCTACAGC | |
| | | CTCAGCAGCACCCTGACGCTGAGCAAAGCAGACTACGAGA | |
| S564- | HC-DNA | CAGGTGCGGCTGCAGGAGTCGGGCCCAGGACTGGTGAAGCCTTCACA | 1757 |
| 105 | | GACCCTGTCCCTCACCTGCACTGTCTCTGGTGGCTCCATCAGCAGTGG | |
| | | TAGTTACTACTGGAGCTGGATCCGGCAGCCCGCCGGGAAGGGACTGG | |
| | | AGTGGATTGGGCGTTTCCATACCAGTGGGAGCACCAACTACAATCCC | |
| | | TCCCTCAAGAGTCGAGTCACCATATCAGTAGACACGTCCAAGAACCA | |
| | | GTTCTCCCTGAAGCTGAGTTCTGTGACCGCCGCAGACACGGCCGTGTA | |
| | | TTACTGTGCGAGAGATTTAAAGGGAAAGACGTGGATACAGACCCCCT | |
| | | TTGACTACTGGGGCCAGGGAATCCTGGTCACCGTCTCCTCAGCCTCCA | |
| | | CCAAGGGCCCATCTGTCTTCCCCCTGGCACCCTCCTCCAAGAGCACCT | |
| | | CTGGGGGCACAGCGGCCCTGGGCTGCCTGGTCAAGGACTACTTCCCC | |
| | | GAACCGGTGACGGTGTCGTGGAACTCAGGCGCTCTGACCAGCGGCGT | |
| | | GCACACCTTCCCGGCTGTCCTACAGTCCTCAGGA | |
| | LC-DNA | CAGTCTGCCCTGACTCAGCCTGCCTCCGTGTCTGGGTCTCCTGGACAG | 1758 |
| | | TCGATCACCATCTCCTGCACTGGAACCAGCAGTGACGTTGGTGCTTAT | |
| | | AACTATGTCTCCTGGTACCAACAGCACCCAGGCAAAGCCCCCAAACT | |
| | | CATGATTTATGAGGTCAGTAATCGGCCCTCAGGGGTTTCTAATCGCTT | |
| | | CTCTGGCTCCAAGTCTGGCAACACGGCCTCCCTGACCATCTCTGGGCT | |
| | | CCAGGCTGAGGACGAGGCTGATTATTACTGCAGCTCATATACAAGCA | |
| | | GCACCTTCTTCGGAACTGGGACCACGGTCACCGTCCTAGGTCAGCCCA | |
| | | AGGCCAACCCCACTGTCACTCTGTTCCCGCCCTCCTCTGAGGAGCTCC | |
| | | AAGCCAACAAGGCCACACTAGTGTGTCTGATCAGTGACTTCTACCCG | |
| | | GGAGCTGTGACAGTGGCCTGGAAGGCAGATGGCAGCCCCGTCAAGGC | |
| | | GGGAGTGGAGACCACCACACCCTCCAAACAAAGCAACAACAAGTAC | |
| | | GCGGCCAGCAGCTAC | |
| S564- | HC-DNA | CAGGTGCAGCTGGTGCAGTCTGGGGCTGAGGTGAAGAAGCCTGGGGC | 1765 |
| 134 | | CTCAGTGAAGGTCTCCTGCAAGGCTTCTGGATACACCTTCACCGGCTA | |
| | | CTATATGCACTGGGTGCGACAGGCCCCTGGACAAGGGCTTGAGTGGA | |
| | | TGGGATGGATCAACCCTAACAGTGGTGGCACAAACTATGCACAGAAG | |
| | | TTTCAGGGCAGGGTCACCATGACCAGGGACACGTCCATCAACACAGC | |
| | | CTACATGGAGCTGAGCAGGCTGAGATCTGACGACACGGCCGTGTATT | |
| | | ACTGTACGAGAGTCGGGAGGTTTTCGATTTTTGGAGTGGAGCTTGACT | |
| | | ACTGGGGCCAGGGAACCCTGGTCACCGTCTCCTCAGCCTCCACCAAG | |
| | | GGCCCATCTGTCTTCCCCCTGGCACCCTCCTCCAAGAGCACCTCTGGG | |
| | | GGCACAGCGGCCCTGGGCTGCCTGGTCAAGGACTACTTCCCCGAACC | |
| | | GGTGACGGTGTCGTGGAACTCAGGCGCCCTGACCAGCGGCGTGCACA | |
| | | CCTTCCCGGCTGTCCTACAGTCCTCAGGA | |
| | LC-DNA | CAGTCTGCCCTGACTCAACCTCCCTCCGCGTCCGGGTCTCCTGGACAG | 1766 |
| | | TCAGTCACCATCTCCTGCACTGGAACCAGCAGTGACGTTGGTGGTTAT | |
| | | AACTATGTCTCCTGGTACCAGCAACACCCAGGCAAAGCCCCCAAACT | |
| | | CATGATTTATGAGGTCAATAAGCGGCCCTCAGGGGTCCCTGATCGCTT | |
| | | CTCTGGCTCCAAGTCTGGCAACACGGCCTCCCTGACCGTCTCTGGGCT | |
| | | CCAGGCTGACGATGAGGCTGATTATTACTGCAGCTCATATGCAGGCA | |
| | | GCAACAATTTGGTTTTCGGCGGAGGGACCAAGCTGACCGTCCTAGGT | |
| | | CAGCCCAAGGCTGCCCCCTCGGTCACTCTGTTCCCGCCCTCCTCTGAG | |
| | | GAGCTTCAAGCCAACAAGGCCACACTGGTGTGTCTCATAAGTGACTT | |
| | | CTACCCGGGAGCCGTGACAGTGGCCTGGAAGGCAGATAGCAGCCCCG | |
| | | TCAAGGCGGGAGTGGAGACCACCACACCCTCCAAACAAAGCAACAA | |
| | | CAAGTACGCGGCCAGCAGCTA | |
| S564- | HC-DNA | CAGGTGCTCCTGGTGCAGTCTGGGGCTGAGGTGAAGAAGCCTGGGGC | 1767 |
| 138 | | CTCAGTGAAGGTCTCCTGCAAGGCTTCTGGATACACCTTCACCGGCTA | |
| | | CTATCTGCACTGGGTGCGACAGGCCCCTGGACAAGGGCTTGAGTGGA | |
| | | TGGGATGGATCAACCCTATCAGTGGTGGCACAAACTATGCACAGAAT | |
| | | TTTCAGGACAGGGTCACCATGACCAGGGACACGTCCATCATCACAGC | |
| | | CTACATGGAACTGAGCAGGCTGAGATCTGACGACACGGCCGTGTATT | |
| | | ACTGTGCGAGACTTGCCTATTATTATGATAGTAGTGCTTACCGGGGTG | |
| | | CTTTTGATATCTGGGGCCAAGGGACAATGGTCACCGTCTCTTCAGCCT | |
| | | CCACCAAGGGCCCATCTGTCTTCCCCCTGGCACCCTCCTCCAAGAGCA | |
| | | CCTCTGGGGGCACAGCGGCCCTGGGCTGCCTGGTCAAGGACTACTTC | |
| | | CCCGAACCGGTGACGGTGTCGTGGAACTCAGGCGCCCTGACCAGCGG | |
| | | CGTGCACACCTTCCCGGCTGTCCTACAGTCCTCAGG | |
| | LC-DNA | CAGTCTGCCCTGACTCAGCCTGCCTCCGTGTCTGGGTCTCCTGGACAG | 1768 |
| | | TCGATCACCATCTCCTGCACTGGAACCAGCAGTGACGTTGGTGGTTAT | |
| | | AACTATGTCTCCTGGTACCAACAGCACCCAGGCAAAGCCCCCAAACT | |
| | | CATGATTTATGAGGTCAGTAATCGGCCCTCAGGGGTTTCTGATCGCTT | |
| | | CTCTGGCTCCAAGTCTGGCAACACGGCCTCCCTGACCATCTCTGGGCT | |
| | | CCAGGCTGAGGACGAGGCTGATTATTACTGCAGCTCATATACAAGCA | |
| | | GCAGCACTTATGTCTTCGGAACTGGGACCAAGGTCACCGTCCTAGGTC | |
| | | AGCCCAAGGCCAACCCCACTGTCACTCTGTTCCCGCCCTCCTCTGAGG | |
| | | AGCTCCAAGCCAACAAGGCCACACTAGTGTGTCTGATCAGTGACTTCT | |
| | | ACCCGGGAGCTGTGACAGTGGCCTGGAAGGCAGATGGCAGCCCCGTC | |
| | | AAGGCGGGAGTGGAGACCACCAAACCCTCCAAACAGAGCAACAACA | |
| | | AGTACGCGGCCAGCAGCTA | |
| S564- | HC-DNA | CAGGTGCAGCTGGTGGAGTCTGGGGGAGGCGTGGTCCAGCCTGGGAG | 1769 |
| 152 | | GTCCCTGAGACTCTCCTGTGCAGCGTCTGGATTCACCTTCAGTTACTA | |
| | | TGGCATGCACTGGGTCCGCCAGGCTCCAGGCAAGGGGCTGGAGTGGG | |
| | | TGGCAGTTATATGGTATGATGGAAGTAATAAACACTATGCAGACTCC | |
| | | GTGAAGGGCCGATTCACCATCTCCAGAGACAATTCCAAGAACACGCT | |
| | | GTATCTGCAAATGAACAGCCTGAGAGCCGAGGACACGGCTGTGTACT | |
| | | ACTGTGCGAAAAATGCGGCCCCCTATTGTAGTGGTGGTAGCTGCTAC | |
| | | GGTACCTACTTTGACTACTGGGGCCAGGGAACCCTGGTCACCGTCTCC | |
| | | TCAGCCTCCACCAAGGGCCCATCTGTCTTCCCCCTGGCACCCTCCTCC | |
| | | AAGAGCACCTCTGGGGGCACAGCGGCCCTGGGCTGCCTGGTCAAGGA | |
| | | CTACTTCCCCGAACCGGTGACGGTGTCGTGGAACTCAGGCGCCCTGA | |
| | | CCAGCGGCGTGCACACCTTCCCGGCTGTCCTACAGTCCTCAGGA | |
| | LC-DNA | GACATCCAGATGACCCAGTCTCCATCCTCCCTGTCTGCATCTGTAGGA | 1770 |
| | | GACAGAGTCACCATCACTTGCCAGGCGAGTCAGGACATTAACAACTA | |
| | | TTTAAATTGGTATCAGCAGAAACCAGGGAAAGCCCCTAAGCTCCTGA | |
| | | TCTACGATGCATCCAATTTGGAAACAGGGGTCCCATCAAGGTTCAGT | |
| | | GGGAGTGGATCTGGGACAGATTTTACTTTCACCATCAGCAGCCTGCA | |
| | | GCCTGAAGATATTGCAACATATTACTGTCAACAGTATGACAATGTCCC | |
| | | TCCGCACACTTTTGGCCAGGGGACCAAGCTGGAGATCAAACGAACTG | |
| | | TGGCTGCACCATCTGTCTTCATCTTCCCGCCATCTGATGAGCAGTTGA | |
| | | AATCTGGAACTGCCTCTGTTGTGTGCCTGCTGAATAACTTCTATCCCA | |
| | | GAGAGGCCAAAGTACAGTGGAAGGTGGATAACGCCCTCCAATCGGGT | |
| | | AACTCCCAGGAGAGTGTCACAGAGCAGGACAGCAAGGACAGCACCT | |
| | | ACAGCCTCAGCAGCACCCTGACGCTGAGCAAAGCAGACTACGAGAA | |
| S564- | HC-DNA | CAGGTCCAGCTGGTGCAGTCTGGGGCTGAGGTGAAGAAGCCTGGGTC | 1771 |
| 218 | | CTCGGTGAAGGTCTCCTGCAAGGCTTCTGGAGGCACCTTCAGCAGCTA | |
| | | TGCTATCAGCTGGGTGCGACAGGCCCCTGGACAAGGGCTTGAGTGGA | |
| | | TGGGAGGGATCATCCCTATCTTTGGTACAGCAAAGTACGCACAGAAG | |
| | | TTCCAGGGCAGAGTCACGATTACCGCGGACGAATCCACGAGCACAGC | |
| | | CTACATGGAGCTGAGCAGCCTGAGATCTGAGGACACGGCCGTGTATT | |
| | | ACTGTGCGAGAGGAAAAGATGGCTACAATCCCTGGGGCGCTTTTGAT | |
| | | ATCTGGGGCCAAGGGACAATGGTCACCGTCTCTTCAGGGAGTGCATC | |
| | | CGCCCCAACCCTTTTCCCCCTCGTCTCCTGTGAGAATTCCCCGTCGGA | |
| | | TACGAGCAGCGTG | |
| | LC-DNA | CAGTCTGCCCTGACTCAGCCTCCCTCCGCGTCCGGGTCTCCTGGACAG | 1772 |
| | | TCAGTCACCATCTCCTGCACTGGAACCAGCAGTGACGTTGGTGGTTAT | |
| | | AACTATGTCTCCTGGTACCAACAGCACCCAGGCAAAGCCCCCAAACT | |
| | | CATGATTTATGAGGTCAGTAAGCGGCCCTCAGGGGTCCCTGATCGCTT | |
| | | CTCTGGCTCCAAGTCTGGCAACACGGCCTCCCTGACCGTCTCTGGGCT | |
| | | CCAGGCTGAGGATGAGGCTGATTATTACTGCAGCTCATATGCAGGCA | |
| | | GCAACAATTTCGGGGTATTCGGCGGAGGGACCAAGCTGACCGTCCTA | |
| | | GGTCAGCCCAAGGCTGCCCCCTCGGTCACTCTGTTCCCGCCCTCCTCT | |
| | | GAGGAGCTTCAAGCCAACAAGGCCACACTGGTGTGTCTCATAAGTGA | |
| | | CTTCTACCCGGGAGCCGTGACAGTGGCCTGGAAGGCAGATAGCAGCC | |
| | | CCGTCAAGGCGGGAGTGGAGACCACCACACCCTCCAAACAAAGCAAC | |
| | | AACAAGTACGCGGCCAGCAGCTACCTGAGCCTGACGCCTGAGCAGTG | |
| | | GAAGTCCCAC | |
| S564- | HC-DNA | GAGGTGCAGCTGGTGGAGTCTGGGGGAGGCTTGGTCCAGCCCGGGGG | 1773 |
| 249 | | GTCCCTGAGACTCTCCTGCGTAGCCTCTGGATTCACCTTCAGTGACTA | |
| | | TGCTATGCACTGGGTCCGCCAGGCTCCAGGGAAGGGACTGGAATATA | |
| | | TTGCAGCTATTAGTAGCAATGGGGGTAGGACATATTATGCAGACTCT | |
| | | GTGAAGGACAAATTCACCATCTCCAGAGACAATTCCAAGAACATCTT | |
| | | GTATCTTCACATGGGCAGCCTGAGAGCGGAGGACACGGCTGTGTATT | |
| | | TCTGTGCGAGAGATCCCCAGTCATGGGTGACTTCCACCACAGCCCATT | |
| | | TCCAGCACTGGGGCCAGGGCACCCTGGTCACCGTCTCCTCAGCATCCC | |
| | | CGACCAGCCCCAAGGTCTTCCCGCTGAGCCTCTGCAGCACCCAGCCA | |
| | | GATGGGAACGTGGTCATCGCCTGCCTGGTCCAGGGCTTCTTCCCCCAG | |
| | | GAGCCACTCAGTGTGACCTGGAGCGAAAGCGGACAGGGCGTGACCGC | |
| | | CAGAAACTTCCC | |
| | LC-DNA | CAGTCTGCCCTGACTCAGCCTGCCTCCGTGTCTGGGTCTCCTGGACAG | 1774 |
| | | TCGATCACCATCTCCTGCACTGGAACCAGCAGTGACATTGGTGGTTAT | |
| | | AACTATGTCTCCTGGTACCAACAACACCCAGGCAAAGCCCCCAAACT | |
| | | CATCATTTCTGATGTCTCTAATCGGCCCTCAGGGGTTTCTAGTCGCTTC | |
| | | TCTGGCTCCAAGTCTGGCAACACGGCCTCCCTGACCATCTCTGGACTC | |
| | | CAGACTGAGGACGAGGCTCATTATTATTGCAGCTCGTTTAGAAGTGG | |
| | | CATCACTCTCGGGGTATTCGGCGGGGGGACCAAGCTGACCGTCCTAG | |
| | | GTCAGCCCAAGGCTGCCCCCTCGGTCACTCTGTTCCCGCCCTCCTCTG | |
| | | AGGAGCTTCAAGCCAACAAGGCCACACTGGTGTGTCTCATAAGTGAC | |
| | | TTCTACCCGGGAGCCGTGACAGTGGCCTGGAAGGCAGATAGCAGCCC | |
| | | CGTCAAGGCGGGAGTGGAGACCACCACACCCTCCAAACAAAGCAAC | |
| | | AACAAGTACGCGGCCAGCAGCTA | |
| S564- | HC-DNA | CAGGTGCAGCTGGTGCAGTCTGGGGCTGAGGTGAAGAAGCCTGGGGC | 1775 |
| 265 | | CTCAGTGAAGGTCTCCTGCAAGGCTTCTGGATACACCTTCACCGGCTA | |
| | | CTATATGCACTGGGTGCGTCAGGCCCCTGGACAAGGGCTTGAGTGGA | |
| | | TGGGATGGATCAACCCTAACAGTGGTGCCATAAACTATGCACAGAAG | |
| | | TTTCAGGGCAGGGTCACCATGACCAGGGACACGTCCATCAGCACAGC | |
| | | CTACATGGAGCTGAGCAGCCTGAGATCTGACGACACGGCCGTGTATT | |
| | | ACTGTGCGAGAGTCGGGAGGTTTTCGATTTTTGGAGTGGAGCTTGATA | |
| | | ACTGGGGCCAGGGAACCCTGGTCACCGTCTCCTCAGCCTCCACCAAG | |
| | | GGCCCATCTGTCTTCCCCCTGGCACCCTCCTCCAAGAGCACCTCTGGG | |
| | | GGCACAGCGGCCCTGGGCTGCCTGGTCAAGGACTACTTCCCCGAACC | |
| | | GGTGACGGTGTCGTGGAACTCAGGCGCCCTGACCAGCGGCGTGCACA | |
| | | CCTTCCCGGCTGTCCTACAGTCCTCAGGA | |
| | LC-DNA | CAGTCTGCCCTGACTCAGCCTCCCTCCGCGTCCGGGTCTCCTGGACAG | 1776 |
| | | TCAGTCACCATCTCCTGCACTGGAACCAGCAGTGACGTTGGTGGTTAT | |
| | | AACTTTGTCTCCTGGTACCAACAGCACCCAGGCAAAGCCCCCAAACT | |
| | | CATGATTTATGAGGTCAGTAAGCGGCCCTCAGGGGTCCCTGATCGCTT | |
| | | CTCTGGCTCCAAGTCTGGCAACACGGCCTCCCTGACCGTCTCTGGGCT | |
| | | CCAGGCTGAGGATGAGGCTGATTATTACTGCAGCTCATATGGAGGCA | |
| | | GCAACAATTTGATATTCGGCGGAGGGACCAGGCTGACCGTCCTAGGT | |
| | | CAGCCCAAGGCTGCCCCCTCGGTCACTCTGTTCCCGCCCTCCTCTGAG | |
| | | GAGCTTCAAGCCAACAAGGCCACACTGGTGTGTCTCATAAGTGACTT | |
| | | CTACCCGGGAGCCGTGACAGTGGCCTGGAAGGCAGATAGCAGCCCCG | |
| | | TCAAGGCGGGAGTGGAGACCACCACACCCTCCAAACAAAGCAACAA | |
| | | CAAGTACGCGGCCAGCAGCTACCTGAGCCTGACGCCTGAGCAGTGGA | |
| | | AGTCCCAC | |
| S564- | HC-DNA | CAGGTGCAGCTGCAGGAGTCGGGCCCAGGACTGGTGAAGCCTTCGGA | 1777 |
| 275 | | GACCCTGTCCCTCACCTGCACTGTCTCTGGTGGCTCCATCAGTAGTTA | |
| | | CTACTGGAGCTGGATCCGGCAGCCCCCAGGGAAGGGACTGGAGTGGA | |
| | | TTGGGTATATCTATTACAGTGGGAGCACCAAGTACAACCCCTCCCTCA | |
| | | AGAGTCGAGTCACCATATCAGTAGACACGTCCAAGAAGCAGTTCTCC | |
| | | CTGAAGTTGAGCTCTGTGACCGCCGCAGACACGGCCGTGTATTACTGT | |
| | | GCGAGACATATAAAGATAGGAGTGGTCGGAGGCCTTACTTTTGACTT | |
| | | CTGGGGCCAGGGAACCCTGGTCACCGTCTCCTCAGGGAGTGCATCCG | |
| | | CCCCAACCCTTTTCCCCCTCGTCTCCTGTGAGAATTCCCCGTCGGATA | |
| | | CGAGCAGCGTG | |
| | LC-DNA | GACATTCAGATGACCCAGTCTCCATCCTCCCTGTCTGCATCTATAGGA | 1778 |
| | | GACAGAGTCACCATCACTTGCCGGGCAAGTCAGAGCATTAGCACCTA | |
| | | TTTAAATTGGTATCAGCAGAAACCAGGGAAAGCCCCTAAACTCCTGA | |
| | | TCTATGCTGCATCCAGTTTGCAAAGTGGGGTCCCATCAAGGTTCAGTG | |
| | | GCAGTGGATCTGGGGCAGATTTCACTCTCACCATCAGCAGTCTGCAAC | |
| | | CTGAAGATTTTGCAACTTACTACTGTCAACAGAGTTACAGTACCCCGC | |
| | | TCACTTTCGGCGGAGGGACGAAGGTGGAGATCAAACGAACTGTGGCT | |
| | | GCACCATCTGTCTTCATCTTCCCGCCATCTGATGAGCAGTTGAAATCT | |
| | | GGAACTGCCTCTGTTGTGTGCCTGCTGAATAACTTCTATCCCAGAGAG | |
| | | GCCAAAGTACAGTGGAAGGTGGATAACGCC | |
| S564- | HC-DNA | CAGGTGCAGCTGGTGCAGTCTGGGGCTGAGGTGAAGAAGCCTGGGGC | 1779 |
| 287 | | CTCAGTGAAGGTCTCCTGCAAGGCTTCTGGATACACCTTCACCGGCTA | |
| | | CTATATGCACTGGGTGCGACAGGCCCCTGGACAAGGGCTTGAGTGGA | |
| | | TGGGATGGATCAACCCTAACAGTGGTGGCACAAACTATGCACAGAAG | |
| | | TTTCAGGGCAGGGTCACCATGACCAGGGACACGTCCATCAGCACAGC | |
| | | CTACATGGAGCTGAGCAGGCTGAGATGTGACGACACGGCCGTGTATT | |
| | | ACTGTGCGAGAGCCTCAACTCCGTATAGCAGTGGCTCCTGGGCGGAC | |
| | | TACTGGGGCCAGGGAACCCTGGTCACCGTCTCCTCAGGGAGTGCATC | |
| | | CGCCCCAACCCTTTTCCCCCTCGTCTCCTGTGAGAATTCCCCGTCGGA | |
| | | TACGAGCAGCGTG | |
| | LC-DNA | CAGTCTGCCCTGACTCAGCCTGCCTCCGTGTCTGGGTCTCCTGGACAG | 1780 |
| | | TCGATCACCATCTCCTGCACTGGAACCAGCAGTGACGTTGGTGGTTAT | |
| | | AACTATGTCTCCTGGTACCAACAACACCCAGGCAAAGCCCCCAAACT | |
| | | CATGATTTATGATGTCAGTAATCGGCCCTCAGGGGTTTCTAATCGCTT | |
| | | CTCTGGCTCCAAGTCTGGCAACACGGCCTCCCTGACCATCTCTGGGCT | |
| | | CCAGGCTGAGGACGAGGCTGATTATTACTGCAGCTCATATGCAAGCA | |
| | | GCAGCACTTGGGTGTTCGGCGGAGGGACCAAGCTGACCGTCCTAGGT | |
| | | CAGCCCAAGGCTGCCCCCTCGGTCACTCTGTTCCCGCCCTCCTCTGAG | |
| | | GAGCTTCAAGCCAACAAGGCCACACTGGTGTGTCTCATAAGTGACTT | |
| | | CTACCCGGGAGCCGTGACAGTGGCCTGGAAGGCAGATAGCAGCCCCG | |
| | | TCAAGGCGGGAGTGGAGACCACCACACCCTCCAAACAAAGCAACAA | |
| | | CAAGTACGCGGCCAGCAGCTATCTGAGCCTGACGCC | |
| S166- | HC-DNA | CAGGTGCAGCTGGTGGAGTCTGGGGGAGGCTTGGTCAAGCCTGGAGG | 1781 |
| 32 | | GTCCCTGAGACTCTCCTGTGCAGCCTCTGGATTCACCTTCAGTGACTA | |
| | | CTACATGAGCTGGATCCGCCAGGCTCCAGGGAAGGGGCTGGAGTGGG | |
| | | TTTCATACATTAGTATTAGTGATACGACCATATACTACGCAGACGCTG | |
| | | TGCAGGGCCGATTCACCATGTCCAGGGACAACGCCAAGAACTCACTG | |
| | | TATCTGCAAATGAACAGCCTGAAGGCCGAGGACACGGCCGTGTATTA | |
| | | CTGTGCGAGAGCTAGCCCATATTGTGGTGGTGATTGCTCTTTCGGCAA | |
| | | TGCTTTTGATATCTGGGGCCTAGGGACAATGGTCACCGTCTCTTCAG | |
| | LC-DNA | GACATCCAGATGACCCAGTCTCCTTCCACCCTGTCTGCATCTGTAGGA | 1782 |
| | | GACAGAGTCACCATCACTTGCCGGGCCAGTCAGAGTATTTTTAGCTGG | |
| | | TTGGCCTGGTATCAGCAGAAACCAGGGAAAGCCCCTAAGCTCCTGAT | |
| | | CTATGATGCCTCCAGTTTGGAAAGTGGGGTCCCATCAAGGTTCAGCG | |
| | | GCAGTGGATCTGGGACAGAATTCACTCTCACCATCAGCAGCCTGCAG | |
| | | CCTGATGATTTTGCAACTTATTACTGCCAACAGTATAATAGTTATTGG | |
| | | ACGTTCGGCCAAGGGACCAAGGTGGAAATCAAAC | |
| S305- | HC-DNA | CAGGTGCAGTTGGTGGAGTCTGGGGGAGGCGTGGTCCAGCCTGGAAG | 1783 |
| 223 | | GTCCCTGAGACTCTCCTGTGCAGCGTCTGGATTCACCTTCAGAAACTT | |
| | | TGGCATGCACTGGGTCCGCCAGGCTCCAGGCAAGGGGCTGGAGTGGG | |
| | | TGGCATTTATATGGACTGCTGAAAGTGATAAATTCTATGCAGACTCCG | |
| | | TGAAGGGCCGATTCACCGTCTCCAGAGACAATTCGAAGAACACGCTG | |
| | | TATTTGGAAATGAACAGCCTGAGAGCCGAGGACACGGCTGTGTATTA | |
| | | CTGTACGAAAGCGATGGACGTCTGGGGCAGAGGGACCACGGTCACCG | |
| | | TCTCCTCAG | |
| | LC-DNA | GAAATTGTGTTGACACAGTCTCCAGCCACCCTGTCTTTGTCTCCAGGG | 1784 |
| | | GAAAGAGCCACCCTCTCCTGCAGGGCCAGTCAGAGTGTTAGCACCTC | |
| | | CTTAGCCTGGTACCAACAGAAATGTGGCCAGGCTCCCCGGCTCCTCAT | |
| | | CTATGATGCATCCAACAGGGCCACTGGCATCCCAGCCAGGTTCAGTG | |
| | | GCAGTGGGTCTGGGACAGACTTCACTCTCACCATCAGCAGCCTAGAG | |
| | | CCTGAAGATTTTGCAGTTTATTACTGTCAACAGCGTGGCAACTGGCCC | |
| | | TTCACTTTCGGCCCTGGGACCAGAGTGGATATCAAAC | |
| S305- | HC-DNA | CAGGTCCAGCTGGTACAGTCTGGGGCTGAGGTGAAGAAGCCTGGGGC | 1785 |
| 399 | | CTCAGTGAAGGTCTCCTGCAAGGTTTCCGGATACACCCTCACTGAATT | |
| | | ATCCATGCACTGGGTGCGACAGGCTCCTGGAAAAGGGCTTGAGTGGA | |
| | | TGGGAGGTTTTGATCCTGAAGATGGTGAAACAATCTACGCACAGAAG | |
| | | TTCCAGGGCAGAGTCACCATGACCGAAGACACATCTACAGACACAGC | |
| | | CTACATGGAGCTGAGCAGCCTGAGATCTGAGGACACGGCCGTGTATT | |
| | | ACTGTGCAACAGGGGGATTGGGTTGTTCTAATGGGGTATGCAACAAC | |
| | | TGGTTCGACCCCTGGGGCCTGGGAACCCTGGTCACCGTCTCCTCAG | |
| | LC-DNA | GAAATAGTGATGACGCAGTCTCCAGCCACCCTGTCTGTGTCTCCAGGG | 1786 |
| | | GAAAGAGCCACCCTCTCCTGCAGGGCCAGTCAGAGTATTACTAGCAA | |
| | | CTTAGCCTGGTACCAGCAGAAACCTGGCCAGGCTCCCAGGCTCCTCAT | |
| | | CTATGGTGCATCCACCAGGGCCACTGGTATCCCAGCCAGGTTCAGTG | |
| | | GCAGTGGGTCTGGGACAGAGTTCACTCTCACCATCAGCAACCTGCAG | |
| | | TCTGAAGATTTTGCAGTTTATTACTGTCAGCAGTATAATAACTGGCCT | |
| | | CTGACGTTCGGCCAAGGGACCAAGGTGGAAATCAAAC | |
| S305- | HC-DNA | CAGGTCCAGCTGGTACAGTCTGGGGCTGAGGTGAAGAAGCCTGGGGC | 1787 |
| 1456 | | CTCAGTGAAGGTCTCCTGCAAGGTTTCCGGATACACCCTCACTGAATT | |
| | | ATCCATGCACTGGGTGCGGCAGGCTCCTGGAAAAGGGCTTGAGTGGA | |
| | | TGGGAGGTTTTGATCCTGAAGATGCTGAAACAATCTACGCACAGAAG | |
| | | TTCCAGGGCAGAGTCACCATGACCGAGGACACATCTACAGACACAGC | |
| | | CTACATGGAGCTGAGCAGCCTGAGATCTGAGGACACGGCCGTGTATT | |
| | | ACTGTGCAACAGGGGGCTTTCCCGTCAATAGCCTTTACGATATTTTGA | |
| | | CTGGTTACCTTGACTACTGGGGCCAGGGAACCCTGGTCACCGTCTCCT | |
| | | CAG | |
| | LC-DNA | GAAATAGTGATGACGCAGTCTCCAGCCACCCTGTCTGTGTCTCCAGGG | 1788 |
| | | GAAAGAGCCACCCTCTCCTGCAGGGCCAGTCAGAATGTTAGCAGCAA | |
| | | CTTAGCCTGGTACCAACAGAAACCTGGCCAGGCTCCCAGGCTCCTCAT | |
| | | CTATGGTGCATCCACCAGGGCCACTGGTATCCCGGCCAGGTTCAGTG | |
| | | GCAGTGGGTCTGGGACAGAGTTCACTCTCACCATCAGCAGCCTGCAG | |
| | | TCTGAAGATTTTGCAGTTTATTACTGTCAGCAGTATAATAACTGGCCT | |
| | | CACACTTTCGGCCCTGGGACCAAAGTGGATATCAAAC | |
| R125- | HC-DNA | CAGGTGCAGATGGTGGAGTCTGGGGGAGGCGTGGTCCAGCCTGGGGG | 1789 |
| 306 | | GTCCCTGAGACTCTCCTGTGCGGTCTCTGGCTTCACCTTCAACAACTTT | |
| | | GGCATGCACTGGGTCCGCCAGGCTCCAGGCAAGGGGCTGGAGTGGGT | |
| | | GGCATTTATTTCATATGAAGGAAGTAAAAAGTCTTATGCAGACTCCGT | |
| | | GAAGGGCCGATTCACCATCTCCAGAGACAGTTCCAAGAACACGTTGT | |
| | | ATCTGCAAATGAACAGCCTGAGACCTGAGGACACGTCTGTCTATTACT | |
| | | GTGCGAAAGAATTAGCGATATTCATGATCTATGCAGGTCGCTACGGTT | |
| | | TGGACGTCTGGGGCCAAGGGACCACGGTCACCGTCTCCTCA | |
| | LC-DNA | GTCTGCCCTGACTCAGCCTGCCTCCGTGTCTGGGTCTCCTGGACAGTC | 1790 |
| | | GATCACCATCTCCTGCACTGGAATCTACAGTGATGTTGATGATTATAC | |
| | | TTCTGTCTCCTGGTACCAACAACACCCAGGCAAAGCCCCCACACTCAT | |
| | | TATTTATGATGTCACTAAGCGGCCCTCAGGGGTTTCCAATCGTTTCTC | |
| | | TGCCTCCAACTCTGACAATACCGCCTCCCTGACAATCTCTGGGCTCCA | |
| | | GGCTGAGGACGAGGCTGAGTATTACTGCTGCTCACGTGGAAGTGCCA | |
| | | CCAATTCTTATGTCTTCGGAACCGGGACCAAGGTCACTGTCCTA | |
| R125- | HC-DNA | CAAGTGCAGCTGCAGGAATCGGGCCCAGGACTGGTGAAGCCTTCGGA | 1791 |
| 444 | | GACCCTGTCCCTCACCTGCAATGTCTCAGGTGGCTCCGTCAAATTTTT | |
| | | CTATTGGAGTTGGATCCGGCAGCCCCCCGGGAAGGGACTGGAGTGGA | |
| | | TTGGATATATCTATTACAGTGGGAGCACCAACTACAACCCCTCCCTCA | |
| | | AGAGTCGAGTGACCATGTCAGTGGACTCGCCCAACAACCAATTCTCC | |
| | | CTGAAACTGAGGTCTGTGACTGCTGCAGACACGGCCGTATATTATTGT | |
| | | GCGAGAGTGGGGGGGGACTGTAGCAGTGGAATATGCCGAACTTATGA | |
| | | CTACTATGCTATGGATGTCTGGGGCCAAGGGACCACGGTCACTGTCTC | |
| | | CTCA | |
| | LC-DNA | CAGTCTGTGCTGACGCAGCCGCCCTCGCTGTCTGGGGCCCCAGGGCA | 1792 |
| | | GAGGGTCACCATCTCCTGCACTGGGAGCCGCTCCAACATCGGGGCTG | |
| | | GCTATGCTGTCCACTGGTACCAGCAACTTCCAGGAACAGCCCCCAAA | |
| | | CTCCTCATCTCCGAGAACACCAATGGGCCCTCAGGAGTCCCTGACCG | |
| | | ATTCTCTGGGTCCAAGTCTGACTCCTCGGCCTCCCTGGCCATCACCGA | |
| | | CCTCCAGGCTGCGGATGAGGCTGATTATTACTGCCAGTCATACGACG | |
| | | GCAGCCTGAGCGGTTGGGTGTTCGGCGGGGGGACCAAACTGACCGTC | |
| | | CT | |
| R3- | HC-DNA | CAGGTCACCTTGAAGGAGTCTGGTCCTGTGCTGGTGAAACCCACAGA | 1793 |
| 428 | | GACCCTCACGCTGACCTGCACCGTCTCTGGGTTCTCACCCAGCAATGC | |
| | | TAGAATGGGTGTGAGCTGGATCCGTCAGCCCCCAGGGAAGGCCCTGG | |
| | | AATGGCTTGCACACGTGTATTCGAATGACGAGAAATCGTACAGCACA | |
| | | TCTCTGAAGAGGAGGCTCACCATCTCCAAGGACACCTCCAAACGGCA | |
| | | GGTGGTCCTTATTATGACCAACTTGGACCCTGCGGACACAGGCACAT | |
| | | ATTACTGTGCACGGGCTCAAGACCCCCGGATACGGTTCGGGGAATTA | |
| | | TTACCCGTCTACTTTGACAACTGGGGCCAGGGAACCCTGGTCACCGTC | |
| | | TCCTCAG | |
| | LC-DNA | GACATCCAGATGACCCAGTCTCCATCCTCCCTGTCTGCATTTGTGGGA | 1794 |
| | | GACAGAGTTACCATCAGTTGCCGGGCAAGTCAGAGCATTGTCAGCTA | |
| | | TTTAAATTGGTATCAGCAGAAACCAGGGAAAGCCCCTAAGCTCCTTTT | |
| | | GTATAGTGCATCCACTTTGCAAAGTGGGGTCCCATCAAGGTTCAGTGG | |
| | | CAGTGGATCTGGGACAGATTTCACTCTCACCATCAGCAGTCTGCAACC | |
| | | TGAAGATTTTGCAACTTACTACTGTCAACAGGGTTACACTACCCCGTG | |
| | | GACGTTCGGCCAAGGGACCAAGGTGGAAATCAAAC | |
| R478 | HC-DNA | GAGGTGCAGCTGTTGGAGTCTGGGGGAGGCTTGGTACTGCCTGGGGG | 1795 |
| 910- | | GTCCCTGAGACTCTCCTGTGCAGCCTCTGGATTCAGCTTTAGCAGCTA | |
| 171 | | TGCCATGAGCTGGGTCCGCCAGGCTCCAGGGAAGGGGCTGGAGTGGG | |
| | | TCTCAGGTATTAGTGGTCGTGGTACTAGCACATACTACGCAGACTCCG | |
| | | TGAAGGGCCGGTTCACCATCTCCAGAGACAATTCCAAGAACACGCTG | |
| | | TATCTGCAAATGAACAGCCTGAGAGCCGAGGACACGGCCGTATATTA | |
| | | TTGTGCGAAAGATCGGGTCAGCTATGGTTCCCCTTACTACTTTGACTA | |
| | | CTGGGGCCAGGGAACGCTGGTCACCGTCTCCTCAG | |
| | LC-DNA | GACATTGTGTTGACGCAGTCTCCAGCCACCCTGTCTTTGTCTCCAGGG | 1796 |
| | | GAAAGAGCCACCCTCTCCTGCGGGGCCAGTCAGAGTGTTAGCAGCAA | |
| | | CTACTTAGCCTGGTACCAGCAGGAACCTGGCCTGGCGCCCAGGCTCCT | |
| | | CATCTATGATGCATCCAGCAGGGCCACTGGCATCCCAGACAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTATGGTAGCTCA | |
| | | TCCACCTTCGGCCAAGGGACACGACTGGAGATTAAAC | |
| R478 | HC-DNA | CAGGTGCAGCTGGTGGAGTCTGGGGGAGGCGTGGTCCAGCCTGGGAG | 1797 |
| 910- | | GTCCCTGAGACTCTCCTGTGCAGCGTCTGGATTCACCTTCAGTAGCTA | |
| 23 | | TGGCATGCACTGGGTCCGCCAGGCTCCAGGCAAGGGGCTGGAGTGGG | |
| | | TGGCAGTTATATGGTATGATGGAAGTAATAAATACTATGCAGACTCC | |
| | | GTGAAGGGCCGATTCACCATCTCCAGAGACAATTCCAAGAACACGCT | |
| | | GTATCTGCAAATGAACAGCCTGAGAGCCGAGGACACGGCTGTGTATT | |
| | | ACTGTGCGAGAGCTCCTGACTACTGGGGCCAGGGAACCCTGGTCACC | |
| | | GTCTCCTCAG | |
| | LC-DNA | GCCATCCAGATGACCCAGTCTCCATCCTCCCTGTCTGCATCTGTAGGA | 1798 |
| | | GACAGAGTCACCATCACTTGCCGGGCAAGTCAGGGCATTAGAAATGA | |
| | | TTTAGGCTGGTATCAGCAGAAACCAGGGAAAGCCCCTAAGCTCCTGA | |
| | | TCTATGCTGCATCCAGTTTACAAAGTGGGGTCCCATCAAGGTTCAGCG | |
| | | GCAGTGGATCTGGCACAGATTTCACTCTCACCATCAGCAGCCTGCAGC | |
| | | CTGAAGATTTTGCAACTTATTACTGTCTACAAGATTACAATTACCCGT | |
| | | ACACTTTTGGCCAGGGGACCAAGCTGGAGATCAAAC | |
| R478 | HC-DNA | GAGGTGCAGCTGTTGGAGTCTGGGGGAGGCTTGGTACAGCCTGGGGG | 1799 |
| 910- | | GTCCCTGAGACTCTCCTGTGCAGTCTCTGGATTCACCGTTAGTAACTA | |
| 25 | | TGGCCTGAGCTGGGTCCGCCAGGGTCCAGGGAAGGGGCTAGAGTGGG | |
| | | TCGCAGCTATTAGTGGTAGTGGTGGTAGGACATACTATGCAGACTCC | |
| | | GTGAAGGGCCGGTTCACCATCTCCAGAGACAATTCCAAGAACACGCT | |
| | | GTTTCTGCAATTGAACAGCCTGAGAGCCGAGGACACGGCCGTATATT | |
| | | ACTGTGCGAAAGGTCGAGATGAACTGGTGGTAGGTGCTACTCAGGAC | |
| | | TACTGGGGCCAGGGAACCCTGGTCACCGTCTCCTCAG | |
| | LC-DNA | GACATCCAGATGACCCAGTCTCCATCCTCCCTGTCTGCATCTGTAGGG | 1800 |
| | | GACAGAGTCACCATCACTTGCCGGGCAAATCAGAACATTAGGAGCTA | |
| | | TTTAAATTGGTATCAGCAGACACCAGGGAAAGCCCCTAAACTCCTGA | |
| | | TCTATGCTACATCCAGTTTGCAAAGTGGGGTCCCATCAAGGTTCAGTG | |
| | | GCAGTGGATCTGGGACACAGTTCACTCTCACCATCAGCAGTCTGCAA | |
| | | CCTGAAGATTTTGCAACTTACTACTGTCAACAGAGTTACAGTATCCCA | |
| | | TTCGATTTCGGCCCTGGGACCAAAGTGGATATCAAAC | |
| R478 | HC-DNA | GAGGTGCAGCTGTTGGAGTCTGGGGGAGACTTAGTACAGCCTGGGGG | 1801 |
| 910-3 | | GTCCCTGAGACTCTCCTGTGTAGCCTCTGGATTCACCTTTAGGAGTTA | |
| | | TGCCATGACCTGGGTCCGCCAGGCTCCAGGGAAGGGGCTGGAGTGGG | |
| | | TCTCAAGTATTAGTGGTAGTGGTGGCGGCACATACTACGCAGACTCC | |
| | | GTGAAGGGCCGGTTCACCATCTCCAGAGACAATTCCAAGAACACGCT | |
| | | GTTTCTGCAAATGAACAGCCTGAGAGCCGAGGACACGGCCGTATATT | |
| | | ACTGTGCGAGAGGGAGGGAGGACTGGTTATTAAGCCTAACATATGGC | |
| | | TACTGGGGCCAGGGAGCCCTGGTCACCGTCTCCTCAG | |
| | LC-DNA | GACATCCAGATGACCCAGTCTCCATCCTCCCTGTCTGCATCTGTAGGA | 1802 |
| | | GACAGAGTCCCCATCACTTGCCGGGCAAGTCAGAGCATTAGCAGCTA | |
| | | TTTAAATTGGTATCAGCAGAGACCAGGGAAAGCCCCTAAGCTCCTGA | |
| | | TCTATGCTGCATCCAGTTTGCAAAGTGGGGTCCCATCAAGGTTCAGTG | |
| | | GCAGTGGATCTGGGACAGATTTCACTCTCACCATCAGCAGTCTGCAAC | |
| | | CTGAAGATTTTGCAACTTACTACTGTCAACAGAGTTACACTATCCCTC | |
| | | CGACTTTCGGCGGAGGGACCAAGGTGGAGATCAAAC | |
| R478 | HC-DNA | GAGGTGCAGCTGGTGGAGTCTGGGGGAGGCTTGGTCCAGCCTGGGGG | 1803 |
| 910- | | GTCCCTGAGACTCTCCTGTGCAGCCTCTGGATTCACCTTTGGTAGCTA | |
| 421 | | TTGGATGAACTGGGTCCGCCAGGCTCCAGGGAAGGGGCTGGAGTGGG | |
| | | TGGCCAACATAAACGAAGATGGAAGTGAGAAATACTATGTGGACTCT | |
| | | GTGAAGGGCCGATTCACCATCTCCAGAGACAACGCCAAGAACTCACT | |
| | | ATATCTGCAAATGAACAGCCTGAGAGCCGAGGACACGGCTGTGTATT | |
| | | ACTGTGCGAGAGGACATTCTCTGGGTGAGTGGGGCCAGGGATCCCCG | |
| | | GTCACCGTCTCCTCAG | |
| | LC-DNA | TCTTCTGAGCTGACTCAGGACCCTGCTGTGTCTGTGGCCTTGGGACAG | 1804 |
| | | ACAGTCAGGATCACATGCCAAGGAGACAGCCTCAGAAGCTATTCTGC | |
| | | AAGCTGGTACCAGCAGAAGCCAGGACAGGCCCCTGTACTTGTCATCT | |
| | | ATATTAAAAACAAACGACCCTCAGGGATCCCAGACCGATTCTCTGGC | |
| | | TCCAGCTCAGGAAACACAGCTTCCTTGACCATCACTGGGGCTCAGGC | |
| | | GGAAGATGAGGCTGACTATTACTGTAACTCCCGGGACAGCAGTGGTG | |
| | | ACCATCTGGTGTTCGGCGGAGGGACCAAGCTGACCGTCCTAG | |
| R478 | HC-DNA | GAGGTGCAGCTGGTGGAGTCTGGGGGAGGCTTGGTACAGCCTGGGGG | 1805 |
| 910-8 | | GTCCCTGAGACTCTCCTGTGCAGCCTCTGGATTCACCTTCAGTAGCTA | |
| | | TAGCATGAACTGGGTCCGCCAGGCTCCAGGGAAGGGGCTGGAGTGGG | |
| | | TTTCATACATTAGTAGTAGTAGTAGTACCATATACTACGCAGACTCTG | |
| | | TGAAGGGCCGATTCACCATCTCCAGAGACAACGCCAAGAACTCACTG | |
| | | TATCTGCAAATGAACAGCCTGAGAGCCGAGGACACGGCTGTGTATTA | |
| | | CTGTGCGAGGGCCAACTGGAACGACATGTACTTCGATCTCTGGGGCC | |
| | | GTGGCACCCTGGTCACTGTCTCCTCAG | |
| | LC-DNA | GAAATTGTGTTGACGCAGTCTCCAGGCACCCTGTCTTTGTCTCCAGGG | 1806 |
| | | GAAAGAGCCACCCTCTCCTGCAGGGCCAGTCAGAGTGTTAGCAGCAG | |
| | | CTACTTAGCCTGGTACCAGCAGAAACCTGGCCAGGCTCCCAGGCTCCT | |
| | | CATCTATGGTGCATCCAGCAGGGCCACTGGCATCCCAGACAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTATGGTAGCTCA | |
| | | CCTTTTGGCCAGGGGACCAAGCTGGAGATCAAAC | |
| S195- | HC-DNA | CAGGTGCAGCTGGTGGAGTCTGGGGGAGGCGTGGTCCAGCCTGGGAG | 1807 |
| 637 | | GTCCCTGAGACTCTCCTGTGCAGCCTCTGGATTCACCTTCAGTAGCAA | |
| | | TGCTATGCACTGGGTCCGCCAGGCTCCAGGCAAGGGGCTAGAGTGGG | |
| | | TGGCAGTTATATCATATGATGGAGATAATAAATACTACGCAGACTCC | |
| | | GTGAAGGGCCGATTCACCATCTCCAGAGACAATTCCAAGAATACGCT | |
| | | GTATCTGCAAATGAACAGCCTGAGAACTGAGGACACGGCTGTGTATT | |
| | | ACTGTGCGAGGTCGTTGGGCGGAAACTACTTCTACGGTATGGACGTCT | |
| | | GGGGCCAAGGGACCACGGTCACCGTCTCCTCA | |
| | LC-DNA | GAAATTGTGTTGACGCAGTCTCCAGGCACCCTGTCTTTGTCTCCAGGG | 1808 |
| | | GAAAGAGCCACCCTCTCCTGCAGGGCCAGTCAGAGTGTTAGCAGCAG | |
| | | CTACTTAGCCTGGTACCAGCAGAAACCTGGCCAGGCTCCCAGGCTCCT | |
| | | CATCTATGGTGCATCCAGCAGGGCCGCTGGCATCCCAGACAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTTTGGTGGCTCT | |
| | | TGGACGTTCGGCCCAGGGACCAAGGTGGAAATCAAAC | |
| S380- | HC-DNA | GAGGTGCAACTGGTGGAGTCTGGGGGAGGCTTGGTACAGCCTGGGGG | 1809 |
| 1191 | | GTCCCTGAGACTCTCCTGTGCGGCCTCTGGATTCACCTTCAGTGGCTA | |
| | | TATCATGAACTGGGTCCGCCAGGCTCCAGGGAAGGGACTGGAGTGGG | |
| | | TTTCATCCATCAGTGGTGGTAGTATTTCCATATCCTACGCAGGCTCTG | |
| | | TGAAGGGCCGATTCACCATCTCCCGAGACAATGCCAAGAACTCACTG | |
| | | TATCTGCAAATGAACAGCCTGAGAGCCGGGGACTCGGCTGTTTATTA | |
| | | CTGTGCTCTTACGACTTTTGGAGTGGTTACCTCTTATCCCTCCTTTGAC | |
| | | TACTGGGGCCAGGGAACCCTGGTCACCGTCTCCTC | |
| | LC-DNA | CAGTCTGTGCTGACGCAGCCGCCCTCAGTGTCTGGGGCCCCAGGGCA | 1810 |
| | | GAGGGTCACCATCTCCTGCACTGGGAGCAGCTCCAACATCGGGGCAG | |
| | | GTTATGATGTACACTGGTACCAGCAGCTTCCAGGAACAGCCCCCAAA | |
| | | CTCCTCATCTATGGTAACAGTAATCGGCCCTCAGGGGTCCCTGACCGA | |
| | | TTCTCTGGCTCCAAGTCTGGCACCTCAGCCTCCCTGGCCATCACTGGG | |
| | | CTCCAGGCTGAGGATGAGGCTGATTATTACTGCCAGTCCTATGACAGC | |
| | | AGCTTGAGTGGTTATGTGTTCGGCGGAGGGACCGAGCTGACCGTCCT | |
| | | AG | |
| S451- | HC-DNA | CAGGTCCAGCTGGTGCAGTCTGGGGCTGAGGTGAAGAAGCCTGGATC | 1811 |
| 101 | | CTCGGTGAAGGTCTCCTGCAAGGCTTCTGGAGGCACCTTCAGCAACTA | |
| | | TGCTATCAGCTGGGTGCGACAGGCCCCTGGACCAGGGCTTGAGTGGA | |
| | | TGGGAGGGATCATCCCTTTCCTTGGTATAGCAAACTACGCACAGAAG | |
| | | TTCCAGGGCAGAGTCACGATTACCGCGGACAAATCCACGAGCACAGC | |
| | | CTACATGGAGCTGAGCAGCCTGAGATCTGAGGACACGGCCGTGTATT | |
| | | ACTGTGCGAGGGCCCCCGGGTATAGTAGTGTAGGGTCGACAAACTAC | |
| | | TTCGACCCCTGGGGCCAGGGAACCCTGGTCACCGTCTCCTCAG | |
| | LC-DNA | CAGTCTGTGCTGACGCAGCCGCCCTCAGTGTCTGGGGCCCCAGGGCA | 1812 |
| | | GAGGGTCACCATCTCCTGCACTGGGAGCAGCTCCAACATCGGGGCAG | |
| | | GTTATGATGTACACTGGTACCAGCAACTTCCAGGAGCAGCCCCCAAA | |
| | | CTCCTCATCTATGCAAACAGCAATCGGCCCTCAGGGGTCCCTGACCGA | |
| | | TTCTCTGGCTCCAAGTCTGGCACCTCAGCCTCCCTGGCCATCACTGGG | |
| | | CTCCAGGCTGAGGATGAGGCTGATTATTACTGCCAGTCCTATGACAGC | |
| | | AGCCTGAGTGGTTATGTCTTCGGAACTGGGACCAAGGTCACCGTCCTA | |
| | | G | |
| S451- | HC-DNA | CAGGTGCAGCTGCAGGAGTCGGGCCCAGGACTGGTGGAGCCTTCACA | 1813 |
| 11 | | GACCCTGTCCCTCACCTGCACTGTCTCTGGTGGCTCCATCAGCAGTGG | |
| | | TGGTTACTACTGGAGTTGGATCCGCCAGCACCCAGGGAAGGGCCTGG | |
| | | AGTGGATCGGGTACATCTCTTACAGTGGTGGAAGCACCTACTACAAC | |
| | | CCGTCCCTCAAGAGTGTAGTTACCATATCACTAGACACGTCTAAGAAT | |
| | | CAGTTCTCCCTGAAGCTGAGCTCTGTGACTGCCGCGGACACGGCCGTG | |
| | | TATTACTGTGCGAGAGTTTCCTATGGTTCGGGGAGTTTTCGTTTTGACT | |
| | | ACTGGGGCCAGGGAACCCTGGTCACCGTCTCCTCAG | |
| | LC-DNA | CAGTCTGCCCTGACTCAGCCTCGCTCAGTGTCCGGGTCTCCTGGACAG | 1814 |
| | | TCAGTCACCATCTCCTGCACTGGAACCAGCAGTGATGTTGGTGGTTAT | |
| | | AACTATTTCTCCTGGTACCAACACCACGCAGGCAAAGCCCCCAAACT | |
| | | CATGATTTATGATGTCAGTAAGCGGCCCTCAGGGGTCCCTGATCGCTT | |
| | | CTCTGGCTCCAAGTCTGGCAACACGGCCTCCCTGACCATCTCTGGCCT | |
| | | CCAGGCTGAGGATGAGGCTGATTATTACTGCTGCTCATATGCAGGCA | |
| | | CCTACACTTGGGTGTTCGGCGGAGGGACCAAGCTGACCGTCCTAG | |
| S451- | HC-DNA | CAGGTGCAGCTGCAGGAGTCGGGCCCAGGACTGGTGAAGCCTTCGGA | 1815 |
| 1101 | | GACCCTGTCCCTCACCTGCACTGTCTCTGGTGGCTCCATCAATAGTTA | |
| | | CTCCTGGAGCTGGATCCGGCAGCCCGCCGGGAAGGGACTGGAGTGGA | |
| | | TTGGGCGTATCTCTACCAGTGGGAGCACCAACAACAACCCCTCCCTCA | |
| | | AGAGTCGAGTCACCATGTCAGTAGACACGTCCAAGGACCAGTTCTCC | |
| | | CTGAAGCTGACCTCTGTGACCGCCGCGGACACGGCCGTGTATTACTGT | |
| | | GCGAGAATTAATGGGGCAGCAGCTGGGACGCCCTTTGACTACTGGGG | |
| | | CCAGGGAACCCTGGTCACCGTCTCCTCAG | |
| | LC-DNA | GACATCCAGATGACCCAGTCTCCATCCTCCCTGTCTGCATCTGTAGGA | 1816 |
| | | GACAGAGTCACCATCACTTGCCGGGCAAGTCAGAGCATTAGCAACTA | |
| | | TTTAAATTGGTATCAGCAGAAACCAGGGAAAGCCCCTAAGCTCCTGA | |
| | | TCTATGCTGCATCCAGTTTGCAAAGTGGGGTCCCATCAAGGTTCAGTG | |
| | | GCAGTGGATCTGGGACAGATTTCACTCTCACCATCAGCAGTCTGCAAC | |
| | | CTGAAGATTTTGCAACTTACTACTGTCAACAGAGTTACAGGACATTCG | |
| | | GCCAAGGGACCAAGGTGGAAATCAAAC | |
| S451- | HC-DNA | GAAGTGCAGCTGGTGGAGTCTGGGGGAGGCTTGGTACAGCCCGGCAG | 1817 |
| 1439 | | GTCCCTGAGACTCTCCTGTGCAGCCTCTGGATTCACCTTTGATGATTTT | |
| | | GCCATGCACTGGGTCCGGCAAGCTCCAGGGAAGGGCCTGGAGTGGGT | |
| | | CTCAGGTATTAGTTGGAATGGTGGTATCATAGGCTATGCGGACTCTGT | |
| | | GAAGGCCCGATTCACCATCTCCAGAGACAACGCCAAGAACTCCCTGT | |
| | | ATCTGCAAATGAACAGTCTGAGAGCTGAGGACACGGCCTTGTATTAC | |
| | | TGTGCAAAGACCAGGGGGGATTATGATTACGTTTGGGGGAGCCGTTC | |
| | | TTCGAATTACTACTTTGACTACTGGGGCCAGGGAACCCTGGTCACGGT | |
| | | CTCCTCAG | |
| | LC-DNA | GACATCCAGATGACCCAGTCTCCATCCTCCCTGTCTGCATCTGTAGGA | 1818 |
| | | GACAGAGTCACCATCACTTGCCAGGCGAGTCAGGACATTAGCAACTA | |
| | | TTTAAATTGGTATCAGAAGAAACCAGGGAAAGCCCCTAAGCTCCTGA | |
| | | TCTACGATGCAACCAATTTGGAAACAGGGGTCCCATCAAGGTTCAGT | |
| | | GGAAGTGGATCTGGGACAGAGTTTACTTTCACCATCAGCAGCCTGCA | |
| | | GCCTGAAGATATTGCAACATATTATTGTCAACAGTATGATAATGTCCC | |
| | | TCCAATCACTTTCGGCCCTGGGACCAAAGTGGATATGAAAC | |
| S451- | HC-DNA | CAGGTGCAGCTGGTGGAGTCTGGGGGAGGCGTGGTCCAGCCTGGGAG | 1807 |
| 1451 | | GTCCCTGAGACTCTCCTGTGCAGCCTCTGGATTCACCTTCAGTAGCAA | |
| | | TGCTATGCACTGGGTCCGCCAGGCTCCAGGCAAGGGGCTAGAGTGGG | |
| | | TGGCAGTTATATCATATGATGGAGATAATAAATACTACGCAGACTCC | |
| | | GTGAAGGGCCGATTCACCATCTCCAGAGACAATTCCAAGAATACGCT | |
| | | GTATCTGCAAATGAACAGCCTGAGAACTGAGGACACGGCTGTGTATT | |
| | | ACTGTGCGAGGTCGTTGGGCGGAAACTACTTCTACGGTATGGACGTCT | |
| | | GGGGCCAAGGGACCACGGTCACCGTCTCCTCA | |
| | LC-DNA | GAAATTGTGTTGACGCAGTCTCCAGGCACCCTGTCTTTGTCTCCAGGG | 1808 |
| | | GAAAGAGCCACCCTCTCCTGCAGGGCCAGTCAGAGTGTTAGCAGCAG | |
| | | CTACTTAGCCTGGTACCAGCAGAAACCTGGCCAGGCTCCCAGGCTCCT | |
| | | CATCTATGGTGCATCCAGCAGGGCCGCTGGCATCCCAGACAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTTTGGTGGCTCT | |
| | | TGGACGTTCGGCCCAGGGACCAAGGTGGAAATCAAAC | |
| S451- | HC-DNA | GAGCCGCAGCTGGTGGAATCTGGGGGAGGCTTGGTACAGCCGGGGGG | 1819 |
| 1477 | | GTCCCTGAGACTCTCCTGTGCAGGCTCTGGATTCGGCTTCATATCTTA | |
| | | CCCCATGAACTGGGTCCGCCAGGCTCCAGGAAAGGGGCCGGAGTGGA | |
| | | TTTCAAATATTAGGACAACCGCTGAAGGTGGAACCTTTTACGCAGACT | |
| | | CTGTGAAGGGCCGATTCACCATGTCCAGAGACGACGGCAAGACTTCA | |
| | | ATATATCTTCAAATGAACAGCCTGAGAGACGAGGACACGGCTACATA | |
| | | TTACTGTGCGAGAGACTCTTCCTACGGATTTGATCTCTGGGGCCAGGG | |
| | | GACAGTGGTCACCGTCTCCTCAG | |
| | LC-DNA | CAGTCTGCCCTGACTCAGCCTCGCTCAGTGTCCGGGTCTCCTGGACAG | 1820 |
| | | TCAGTCACCATCTCCTGCACTGGAACCAGTAGTGATGTTGGTGGTTAT | |
| | | AACTATGTCTCCTGGTATCAACAACGCCCAGGGAAAGCCCCCGAATT | |
| | | GATGATTTATCATGTCAGTGAGCGGCCCTCAGGGGTCCCTGATCGCTT | |
| | | CTCTGGCTCCAAATCTGGCAACACGGCCTCCCTGACCATCTCTAGGCT | |
| | | CCAGGCTGAGGATGAGGCTGATTATTACTGCTGCTCATATGCAGGCA | |
| | | GCCACTTTTGGGTGTTCGGCGGAGGGACCAAGTTGACCGTCCTAG | |
| S451- | HC-DNA | GAGGTGCAGCTGGTGGAGTCTGGGGGAGGCTTGGTCCAGCCTGGGGG | 1803 |
| 1503 | | GTCCCTGAGACTCTCCTGTGCAGCCTCTGGATTCACCTTTGGTAGCTA | |
| | | TTGGATGAACTGGGTCCGCCAGGCTCCAGGGAAGGGGCTGGAGTGGG | |
| | | TGGCCAACATAAACGAAGATGGAAGTGAGAAATACTATGTGGACTCT | |
| | | GTGAAGGGCCGATTCACCATCTCCAGAGACAACGCCAAGAACTCACT | |
| | | ATATCTGCAAATGAACAGCCTGAGAGCCGAGGACACGGCTGTGTATT | |
| | | ACTGTGCGAGAGGACATTCTCTGGGTGAGTGGGGCCAGGGATCCCCG | |
| | | GTCACCGTCTCCTCAG | |
| | LC-DNA | TCTTCTGAGCTGACTCAGGACCCTGCTGTGTCTGTGGCCTTGGGACAG | 1804 |
| | | ACAGTCAGGATCACATGCCAAGGAGACAGCCTCAGAAGCTATTCTGC | |
| | | AAGCTGGTACCAGCAGAAGCCAGGACAGGCCCCTGTACTTGTCATCT | |
| | | ATATTAAAAACAAACGACCCTCAGGGATCCCAGACCGATTCTCTGGC | |
| | | TCCAGCTCAGGAAACACAGCTTCCTTGACCATCACTGGGGCTCAGGC | |
| | | GGAAGATGAGGCTGACTATTACTGTAACTCCCGGGACAGCAGTGGTG | |
| | | ACCATCTGGTGTTCGGCGGAGGGACCAAGCTGACCGTCCTAG | |
| S451- | HC-DNA | GAGGTGCAACTGGTGGAGTCTGGGGGAGGCTTGGTACAGCCTGGGGG | 1809 |
| 1522 | | GTCCCTGAGACTCTCCTGTGCGGCCTCTGGATTCACCTTCAGTGGCTA | |
| | | TATCATGAACTGGGTCCGCCAGGCTCCAGGGAAGGGACTGGAGTGGG | |
| | | TTTCATCCATCAGTGGTGGTAGTATTTCCATATCCTACGCAGGCTCTG | |
| | | TGAAGGGCCGATTCACCATCTCCCGAGACAATGCCAAGAACTCACTG | |
| | | TATCTGCAAATGAACAGCCTGAGAGCCGGGGACTCGGCTGTTTATTA | |
| | | CTGTGCTCTTACGACTTTTGGAGTGGTTACCTCTTATCCCTCCTTTGAC | |
| | | TACTGGGGCCAGGGAACCCTGGTCACCGTCTCCTC | |
| | LC-DNA | CAGTCTGTGCTGACGCAGCCGCCCTCAGTGTCTGGGGCCCCAGGGCA | 1810 |
| | | GAGGGTCACCATCTCCTGCACTGGGAGCAGCTCCAACATCGGGGCAG | |
| | | GTTATGATGTACACTGGTACCAGCAGCTTCCAGGAACAGCCCCCAAA | |
| | | CTCCTCATCTATGGTAACAGTAATCGGCCCTCAGGGGTCCCTGACCGA | |
| | | TTCTCTGGCTCCAAGTCTGGCACCTCAGCCTCCCTGGCCATCACTGGG | |
| | | CTCCAGGCTGAGGATGAGGCTGATTATTACTGCCAGTCCTATGACAGC | |
| | | AGCTTGAGTGGTTATGTGTTCGGCGGAGGGACCGAGCTGACCGTCCT | |
| | | AG | |
| S451- | HC-DNA | CAGGTGCAACTGCAGGAGTCGGGCCCAGGACTGGTGAAGCCTTCACA | 1821 |
| 1921 | | GACCCTGTCCCTCACCTGCACTGTCTCTGGTGGCTCCATCAGCAGTGG | |
| | | TGATTACTACTGGAGCTGGATCCGCCAGCCCCCAGGGAAGGGCCTGG | |
| | | AGTGGCTTGGGTACATCTATTACAGTGGGAGCACCTTCTACAACCCGT | |
| | | CCCTCAAGAGTCGAGTTACCATATCAGTAGACACGTCCAAGAACCAG | |
| | | TTCTCCCTGAGGCTGACCTCTGTGACTGCCGCAGACACGGCCGTGTAT | |
| | | TTCTGTGCCAGAGAGGAGAATAAATTCAACTATGGCCATCATCCCCTC | |
| | | AATGGAGTCTTTGCCTACTGGGGCCAGGGAACCCTGGTCACCGTCTCC | |
| | | TCAG | |
| | LC-DNA | GACATCGTGATGACCCAGTCTCCAGACTCCCTGGCTGTGTCTCTGGGC | 1822 |
| | | GAGAGGGCCACCATCAACTGCAAGTCCAGCCAGAGTGTTTTGTACAG | |
| | | CCCCAACAATAAGAACTACTTAGCTTGGTACCAGCAGAAACCAGGAC | |
| | | AGCCTCCTAACCTGCTCATCTACTGGGCATCTACCCGGGAATCCGGGG | |
| | | TCCCTGACCGATTCAGTGGCAGCGGGTCTGGGACAGATTTCACTCTCA | |
| | | CCATCAACAGCCTGCAGGCTGAAGATGTGGCAGTTTATTACTGTCAGC | |
| | | AATCTTATAATACTCCTCGGACGTTCGGCCAAGGGACCAAGGTGGAA | |
| | | ATCAAAC | |
| S451- | HC-DNA | CAGGTCACCTTGAAGGAGTCTGGTCCTGTGCTGGTGAAACCCACAGA | 1823 |
| 337 | | GACCCTCACGCTGACCTGCACCGTCTCTGGGTTCTCACTCATCAATGC | |
| | | TAGGTTGGGTGTGAGCTGGATCCGTCAGCCCCCAGGGAAGGCCCTGG | |
| | | AGTGGCTTGCACACATTTTTTCGGATGACGAGAAATCCTACAGCACAT | |
| | | CTCTGAAGAGCAGGCTCACCATCTCCAAGGACACCTCCAAGAGCCAG | |
| | | GTGGTCCTTACCATGACCAACATGGACCCTGTGGACACAGCCACATA | |
| | | TTACTGTGCACGGATATCTTGGCCCCCTTATGGTTCGGGGACTTATTA | |
| | | TATTAAGGCTTTTGATATCTGGGGCCAAGGGACACTGGTCACCGTCTC | |
| | | TTCAG | |
| | LC-DNA | CAGTCTGCCCTGACTCAGCCTGTCTCCGTGTCTGGGTCTCCTGGACAG | 1824 |
| | | TCGATCACCATCTCCTGCACTGGAACCAGCAGTGACGTTGGTGGTTAT | |
| | | AACTATGTCTCCTGGTACCAACAGCACCCAGGCAAAGCCCCCAAACT | |
| | | CATGATTTCTGAGGTCAGTAATCGACCCTCAGGGGTTTCTAATCGCTT | |
| | | CTCTGGCTCCAAGTCTGGCAACACGGCCTCCCTGACCATCTCTGGGCT | |
| | | CCAGGCTGAGGACGAGGCTGATTATTACTGCAGCTCATATGCAAGCA | |
| | | GCAGCACCCTTTGGGTGTTCGGCGGAGGGACCAAGCTGACCGTCCT | |
| S451- | HC-DNA | CAGGTGCAGCTGCAGGAGTCGGGCCCAGGACTGGTGAAGCCTTCGGA | 1825 |
| 650 | | GACCCTGTCCCTCACCTGCACTGTCTCTGGTGCCTCCATCAGTAATTTC | |
| | | TACTGGAGCTGGATCCGGCAGCCCCCAGGGAAGGGACTGGAGTGGAT | |
| | | TGGGTATATCTATTATAGTGGGAGCACCAACTACAACCCCTCCCTCAA | |
| | | GAGTCGAGTCACCATGTCACTAGACACGTCCAAGAACCAGTTCTCCCT | |
| | | GAATCTGAGCTCTGTGACCGCTGCGGACACGGCCGTGTATTACTGTGC | |
| | | GAGAATCCCCAATTTCTGGTTCGGGGAGTTATTATTTGACTTCTGGGG | |
| | | CCACGGAACGCTGGTCACCGTCTCCTCAG | |
| | LC-DNA | GAAATTGTGTTGACGCAGTCTCCAGGCACCCTGTCTTTGTCTCCAGGG | 1826 |
| | | GAAAGAGCCACCCTCTCCTGCAGGGCCAGTCAGAGTGTTAGCAGCAA | |
| | | CTACTTAGCCTGGTACCAGCAGAAACCTGGCCAGGCTCCCAGGCTCCT | |
| | | CATCTATGGTGCATCCAGCAGGGCCACTGGCATCCCAGACAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTATGGTAGCTCA | |
| | | CCTCCGATCACCTTCGGCCAAGGGACACGACTGGAGATTAAAC | |
| S626- | HC-DNA | CAGGTCCAGCTGGTGCAGTCTGGGGCTGAGGTGAAGAAGCCTGGATC | 1811 |
| 362 | | CTCGGTGAAGGTCTCCTGCAAGGCTTCTGGAGGCACCTTCAGCAACTA | |
| | | TGCTATCAGCTGGGTGCGACAGGCCCCTGGACCAGGGCTTGAGTGGA | |
| | | TGGGAGGGATCATCCCTTTCCTTGGTATAGCAAACTACGCACAGAAG | |
| | | TTCCAGGGCAGAGTCACGATTACCGCGGACAAATCCACGAGCACAGC | |
| | | CTACATGGAGCTGAGCAGCCTGAGATCTGAGGACACGGCCGTGTATT | |
| | | ACTGTGCGAGGGCCCCCGGGTATAGTAGTGTAGGGTCGACAAACTAC | |
| | | TTCGACCCCTGGGGCCAGGGAACCCTGGTCACCGTCTCCTCAG | |
| | LC-DNA | CAGTCTGTGCTGACGCAGCCGCCCTCAGTGTCTGGGGCCCCAGGGCA | 1812 |
| | | GAGGGTCACCATCTCCTGCACTGGGAGCAGCTCCAACATCGGGGCAG | |
| | | GTTATGATGTACACTGGTACCAGCAACTTCCAGGAGCAGCCCCCAAA | |
| | | CTCCTCATCTATGCAAACAGCAATCGGCCCTCAGGGGTCCCTGACCGA | |
| | | TTCTCTGGCTCCAAGTCTGGCACCTCAGCCTCCCTGGCCATCACTGGG | |
| | | CTCCAGGCTGAGGATGAGGCTGATTATTACTGCCAGTCCTATGACAGC | |
| | | AGCCTGAGTGGTTATGTCTTCGGAACTGGGACCAAGGTCACCGTCCTA | |
| | | G | |
| S626- | HC-DNA | GAAGTGCAGCTGGTGGAGTCTGGGGGAGGCTTGGTACAGCCCGGCAG | 1817 |
| 651 | | GTCCCTGAGACTCTCCTGTGCAGCCTCTGGATTCACCTTTGATGATTTT | |
| | | GCCATGCACTGGGTCCGGCAAGCTCCAGGGAAGGGCCTGGAGTGGGT | |
| | | CTCAGGTATTAGTTGGAATGGTGGTATCATAGGCTATGCGGACTCTGT | |
| | | GAAGGCCCGATTCACCATCTCCAGAGACAACGCCAAGAACTCCCTGT | |
| | | ATCTGCAAATGAACAGTCTGAGAGCTGAGGACACGGCCTTGTATTAC | |
| | | TGTGCAAAGACCAGGGGGGATTATGATTACGTTTGGGGGAGCCGTTC | |
| | | TTCGAATTACTACTTTGACTACTGGGGCCAGGGAACCCTGGTCACGGT | |
| | | CTCCTCAG | |
| | LC-DNA | GACATCCAGATGACCCAGTCTCCATCCTCCCTGTCTGCATCTGTAGGA | 1818 |
| | | GACAGAGTCACCATCACTTGCCAGGCGAGTCAGGACATTAGCAACTA | |
| | | TTTAAATTGGTATCAGAAGAAACCAGGGAAAGCCCCTAAGCTCCTGA | |
| | | TCTACGATGCAACCAATTTGGAAACAGGGGTCCCATCAAGGTTCAGT | |
| | | GGAAGTGGATCTGGGACAGAGTTTACTTTCACCATCAGCAGCCTGCA | |
| | | GCCTGAAGATATTGCAACATATTATTGTCAACAGTATGATAATGTCCC | |
| | | TCCAATCACTTTCGGCCCTGGGACCAAAGTGGATATGAAAC | |
| S626- | HC-DNA | CAGGTGCAGCTGGTGCAGTCTGGGGCTGAGGTGAAGAAGCCTGGGGC | 1827 |
| 692 | | CTCAGTGAAGGTCTCCTGCAAGGCTTCTGGATACGCCTTCACCAGTTA | |
| | | TGATATCAACTGGGTGCGACAGGCCACTGGACAAGGGCTTGAGTGGA | |
| | | TGGGATGGATGAACCCTAACAGTGGTGACACATTCTATGCACAGAAG | |
| | | TTCCAGGGCAGAGTCACCATGACCAGGAGCACCTCCATAAGCACAGC | |
| | | CTACATGGAGCTGAGCAGCCTGAGATCTGAGGACACGGCCGTGTATT | |
| | | ACTGTGCGAGAGGGAGGGTAGGGGCGGATTATGTTTCGGGGAACCGT | |
| | | GGATACTACTACTACTACTACATGGACGTCTGGGGCAAAGGGACCAC | |
| | | GGTCACCGTCTCCTCA | |
| | LC-DNA | TCTTCTGAGCTGACTCAGGACCCTGCTGTGTCTGTGGCCTTGGGACAG | 1828 |
| | | ACAGTCAGGATCACATGCCAAGGAGAGAACCTCAGAAGCTACTATGC | |
| | | AACCTGGTACCAGCAGAAGCCAGGACAGGCCCCTATACTTGTCATCT | |
| | | ATGGTAAAAACAACCGGCCCTCAGGGATCCCAGACCGATTCTCTGGC | |
| | | TCCAGCTCAGGAAACACAGCTTCCTTGACCATCACTGGGGCTCAGGC | |
| | | GGAAGATGAGGCTGACTATTACTGTAACTCCCGGGACAGCAGTGGTA | |
| | | ACCATCTAAGGGTGTTCGGCGGAGGGACCAAGCTGACCGTCCTAG | |
| S626- | HC-DNA | CAGGTGCAGCTGGTGCAGTCTGGGGCTGAGGTGAAGAAGCCTGGGGC | 1827 |
| 7 | | CTCAGTGAAGGTCTCCTGCAAGGCTTCTGGATACGCCTTCACCAGTTA | |
| | | TGATATCAACTGGGTGCGACAGGCCACTGGACAAGGGCTTGAGTGGA | |
| | | TGGGATGGATGAACCCTAACAGTGGTGACACATTCTATGCACAGAAG | |
| | | TTCCAGGGCAGAGTCACCATGACCAGGAGCACCTCCATAAGCACAGC | |
| | | CTACATGGAGCTGAGCAGCCTGAGATCTGAGGACACGGCCGTGTATT | |
| | | ACTGTGCGAGAGGGAGGGTAGGGGCGGATTATGTTTCGGGGAACCGT | |
| | | GGATACTACTACTACTACTACATGGACGTCTGGGGCAAAGGGACCAC | |
| | | GGTCACCGTCTCCTCA | |
| | LC-DNA | TCTTCTGAGCTGACTCAGGACCCTGCTGTGTCTGTGGCCTTGGGACAG | 1828 |
| | | ACAGTCAGGATCACATGCCAAGGAGAGAACCTCAGAAGCTACTATGC | |
| | | AACCTGGTACCAGCAGAAGCCAGGACAGGCCCCTATACTTGTCATCT | |
| | | ATGGTAAAAACAACCGGCCCTCAGGGATCCCAGACCGATTCTCTGGC | |
| | | TCCAGCTCAGGAAACACAGCTTCCTTGACCATCACTGGGGCTCAGGC | |
| | | GGAAGATGAGGCTGACTATTACTGTAACTCCCGGGACAGCAGTGGTA | |
| | | ACCATCTAAGGGTGTTCGGCGGAGGGACCAAGCTGACCGTCCTAG | |
| S626- | HC-DNA | GAGCCGCAGCTGGTGGAATCTGGGGGAGGCTTGGTACAGCCGGGGGG | 1819 |
| 747 | | GTCCCTGAGACTCTCCTGTGCAGGCTCTGGATTCGGCTTCATATCTTA | |
| | | CCCCATGAACTGGGTCCGCCAGGCTCCAGGAAAGGGGCCGGAGTGGA | |
| | | TTTCAAATATTAGGACAACCGCTGAAGGTGGAACCTTTTACGCAGACT | |
| | | CTGTGAAGGGCCGATTCACCATGTCCAGAGACGACGGCAAGACTTCA | |
| | | ATATATCTTCAAATGAACAGCCTGAGAGACGAGGACACGGCTACATA | |
| | | TTACTGTGCGAGAGACTCTTCCTACGGATTTGATCTCTGGGGCCAGGG | |
| | | GACAGTGGTCACCGTCTCCTCAG | |
| | LC-DNA | CAGTCTGCCCTGACTCAGCCTCGCTCAGTGTCCGGGTCTCCTGGACAG | 1820 |
| | | TCAGTCACCATCTCCTGCACTGGAACCAGTAGTGATGTTGGTGGTTAT | |
| | | AACTATGTCTCCTGGTATCAACAACGCCCAGGGAAAGCCCCCGAATT | |
| | | GATGATTTATCATGTCAGTGAGCGGCCCTCAGGGGTCCCTGATCGCTT | |
| | | CTCTGGCTCCAAATCTGGCAACACGGCCTCCCTGACCATCTCTAGGCT | |
| | | CCAGGCTGAGGATGAGGCTGATTATTACTGCTGCTCATATGCAGGCA | |
| | | GCCACTTTTGGGTGTTCGGCGGAGGGACCAAGTTGACCGTCCTAG | |
| S626- | HC-DNA | CAGGTGCAACTGCAGGAGTCGGGCCCAGGACTGGTGAAGCCTTCACA | 1821 |
| 75 | | GACCCTGTCCCTCACCTGCACTGTCTCTGGTGGCTCCATCAGCAGTGG | |
| | | TGATTACTACTGGAGCTGGATCCGCCAGCCCCCAGGGAAGGGCCTGG | |
| | | AGTGGCTTGGGTACATCTATTACAGTGGGAGCACCTTCTACAACCCGT | |
| | | CCCTCAAGAGTCGAGTTACCATATCAGTAGACACGTCCAAGAACCAG | |
| | | TTCTCCCTGAGGCTGACCTCTGTGACTGCCGCAGACACGGCCGTGTAT | |
| | | TTCTGTGCCAGAGAGGAGAATAAATTCAACTATGGCCATCATCCCCTC | |
| | | AATGGAGTCTTTGCCTACTGGGGCCAGGGAACCCTGGTCACCGTCTCC | |
| | | TCAG | |
| | LC-DNA | GACATCGTGATGACCCAGTCTCCAGACTCCCTGGCTGTGTCTCTGGGC | 1822 |
| | | GAGAGGGCCACCATCAACTGCAAGTCCAGCCAGAGTGTTTTGTACAG | |
| | | CCCCAACAATAAGAACTACTTAGCTTGGTACCAGCAGAAACCAGGAC | |
| | | AGCCTCCTAACCTGCTCATCTACTGGGCATCTACCCGGGAATCCGGGG | |
| | | TCCCTGACCGATTCAGTGGCAGCGGGTCTGGGACAGATTTCACTCTCA | |
| | | CCATCAACAGCCTGCAGGCTGAAGATGTGGCAGTTTATTACTGTCAGC | |
| | | AATCTTATAATACTCCTCGGACGTTCGGCCAAGGGACCAAGGTGGAA | |
| | | ATCAAAC | |
| S626- | HC-DNA | CAGGTGCAGCTGGTGGAGTCTGGGGGAGGCGTGGTCCAGCCTGGGAG | 1829 |
| 8 | | GTCCCTGAGACTCTCCTGTGTATCCTCTGAAGTCACCTTCAATAGATA | |
| | | TACTATGCACTGGGTCCGCCAGGCTCCAGGCAAGGGGCTGGAGTGGG | |
| | | TGGCATCTATATCATTTGAAGGAAGTGTTAAAACCTATGTAGACTCCG | |
| | | TGAAGGGCCGATTCACCATCTCCAGAGACGATTCCAAGAAAACGCTG | |
| | | TTTCTGCAGTTGAACAGCCTGAGAGATGAGGACACGGCTATGTATTA | |
| | | CTGTGCGCGAGGTCAGTGGCCATCCGGGGGTGACTACTGGGGCCGGG | |
| | | GAACGCTGGTCACCGTCTCCTCAG | |
| | LC-DNA | GATGTTGTGCTGACTCAGTCTCCACTCTCCCTGTCCGTCACCCTTGGA | 1830 |
| | | CAGCCGGCCTCCATCTCCTGCAGGTCTAGTCAAAGCCTCGTTTACAGT | |
| | | GATGGAAGCACCTACTTGAATTGGTTTCATCAGAGGCCAGGCCAATC | |
| | | TCCAAGGCGCCTAATTTATAAGGTTTCTAACCGGGACTCTGGGGTCCC | |
| | | AGACAGATTCAGCGGCAGTGGGTCAGGCACTGATTTCACACTGAAAA | |
| | | TCACCAGGGTGGCGGCTGAGGATGTTGGGGTTTATTACTGCATGCAA | |
| | | GGTACATACTGGCCTCCGACGTTCGGCCAAGGGACCAAGGTGGAAAT | |
| | | CAAAC | |
| S68- | HC-DNA | GAGGTGCAGCTGGTGCAGTCTGGAGCAGAAATCAAAAAGCCGGGGG | 1831 |
| 253 | | AGTCTCTGAAGATCTCCTGCCAGGGTTCTGGATACATCTTTACCAACA | |
| | | ACTGGATCGGCTGGGTCCGCCAGCAGCCCGGCAAAGGACTGGAGTGG | |
| | | ATGGGCATCATCTATCCTGGTGACTCTGATGCCAGATATAGCCCGTCC | |
| | | TTCCAAGGCCACGTCAGCTTCTCTGCCGACAAGTCCATCAACACCGCC | |
| | | TTCCTGCAGTGGCACAGCCTGAAGGCCTCGGACACCGCCATGTATTAT | |
| | | TGTGCGAGAATCCGGAGAAGGGGACAGGGAGCTACTGCTGCTTTCGA | |
| | | TATCTGGGGCCCGGGGACAAAGGTCACCGTCTCTTCAG | |
| | LC-DNA | GACATCGTGATGACCCAGTCTCCAGACTCCCTGACTGTGTCTCTGGGC | 1832 |
| | | GAGAGGGCCACCATCAACTGCAAGTCCAGCCAGAATATTTTAACCAC | |
| | | CTCCAACAATAAGAATTACTTGGCTTGGTACCAACAAAAGCCAGGAC | |
| | | AGCCTCCTAAGCTGCTCATTTACTGGGCATCTACCCGGGAATCCGGGG | |
| | | TCCCTGACCGATTCAGTGGCAGCGGGTCTGGGACAGATTTCACTCTCA | |
| | | CCATCAGCAGCCTGCAGGCTGAAGATGTGGCAGTTTATTACTGTCAAC | |
| | | AATATTTTAATTCTCCTCCGTACACTTTTGGCCAGGGGACCAAGCTGG | |
| | | AGATCAAAC | |
| S728- | HC-DNA | CAGGTGCAGCTGGTGCAGTCTGGGGCTGAGGTGAAGAAGCCTGGGGC | 1833 |
| 1502 | | CTCAGTGAAGGTCTCCTGCAAGGCTTCTGGATACACCTTCACCGGCTA | |
| | | CTATATGCACTGGGTGCGACAGGCCCCTGGACAAGGGCTTGAGTGGA | |
| | | TGGGACGGATCAACCCTAACAGTGGTGGCACAAACTATGCACAGAAG | |
| | | TTTCAGGGCAGGGTCACCATGACCAGGGACACGTCCATCAGCACAGC | |
| | | CTACATGGAGCTGAGCAGGCTGAGATCTGACGACACGGCCGTGTATT | |
| | | ACTGTGCGAGAGCTCACCAGCCACTGCTGTACGGGTTGGGCTACTACT | |
| | | TTGACTACTGGGGCCAGGGAACCCTGGTCACCGTCTCCTCAG | |
| | LC-DNA | CAGTCTGCCCTGACTCAGCCTGCCTCCGTGTCTGGGTCTCCTGGACAG | 1834 |
| | | TCGATCACCATCTCCTGCACTGGAACCAGCAGTGACGTTGGTGGTTAT | |
| | | AACTATGTCTCCTGGTACCAACAGCACCCAGGCAAAGCCCCCAAACT | |
| | | CATGATTTATGAGGTCAGTAATCGGCCCTCAGGGGTTTCTAATCGCTT | |
| | | CTCTGGCTCCAAGTCTGGCAACACGGCCTCCCTGACCATCTCTGGGCT | |
| | | CCAGGCTGAGGACGAGGCTGATTATTACTGCAGCTCATATACAAGCA | |
| | | GCAGCACTCTGGTATTCGGCGGAGGGACCAAGCTGACCGTCCTAG | |
| S728- | HC-DNA | GAGGTGCAGCTGGTGGAGTCCGGGGGAGGCTTAGTTCAGCCTGGGGG | 1835 |
| 1789 | | GTCCCTGAGACTCTCCTGTGCAGCCTCTGGATTCACCTTCAGTAGCTA | |
| | | CTGGATGCACTGGGTCCGCCAAGCTCCAGGGAAGGGGCTGGTGTGGG | |
| | | TCTCACGTATTAATAGTGATGGGAGTAGCACAAGCTACGCGGACTCC | |
| | | GTGAAGGGCCGATTCACCATCTCCAGAGACAACGCCAAGAACACGCT | |
| | | GTATCTGCAAATGAACAGTCTGAGAGCCGAGGACACGGCTGTGTATT | |
| | | ACTGTGCAAGAGATCGGTATAGCAGCCTTGACTACTGGGGCCAGGGA | |
| | | ACCCTGGTCACCGTCTCCTCAG | |
| | LC-DNA | GAAATTGTGTTGACGCAGTCTCCAGGCACCCTGTCTTTGTCTCCAGGG | 1836 |
| | | GAAAGAGCCACCCTCTCCTGCAGGGCCAGTCAGAGTGTTAGCAGCAG | |
| | | CTACTTAGCCTGGTACCAGCAGAAACCTGGCCAGGCTCCCAGGCTCCT | |
| | | CATCTATGGTGCATCCAGCAGGGCCACTGGCATCCCAGACAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTATGGTAGCTCA | |
| | | CCCGCGCTCACTTTCGGCGGAGGGACCAAGGTGGAGATCAAAC | |
| S728- | HC-DNA | CAGGTGCAGCTGGTGCAGTCTGGGGCTGAGGTGAAGAAGCCTGGGGC | 1833 |
| 1806 | | CTCAGTGAAGGTCTCCTGCAAGGCTTCTGGATACACCTTCACCGGCTA | |
| | | CTATATGCACTGGGTGCGACAGGCCCCTGGACAAGGGCTTGAGTGGA | |
| | | TGGGACGGATCAACCCTAACAGTGGTGGCACAAACTATGCACAGAAG | |
| | | TTTCAGGGCAGGGTCACCATGACCAGGGACACGTCCATCAGCACAGC | |
| | | CTACATGGAGCTGAGCAGGCTGAGATCTGACGACACGGCCGTGTATT | |
| | | ACTGTGCGAGAGCTCACCAGCCACTGCTGTACGGGTTGGGCTACTACT | |
| | | TTGACTACTGGGGCCAGGGAACCCTGGTCACCGTCTCCTCAG | |
| | LC-DNA | CAGTCTGCCCTGACTCAGCCTGCCTCCGTGTCTGGGTCTCCTGGACAG | 1834 |
| | | TCGATCACCATCTCCTGCACTGGAACCAGCAGTGACGTTGGTGGTTAT | |
| | | AACTATGTCTCCTGGTACCAACAGCACCCAGGCAAAGCCCCCAAACT | |
| | | CATGATTTATGAGGTCAGTAATCGGCCCTCAGGGGTTTCTAATCGCTT | |
| | | CTCTGGCTCCAAGTCTGGCAACACGGCCTCCCTGACCATCTCTGGGCT | |
| | | CCAGGCTGAGGACGAGGCTGATTATTACTGCAGCTCATATACAAGCA | |
| | | GCAGCACTCTGGTATTCGGCGGAGGGACCAAGCTGACCGTCCTAG | |
| S728- | HC-DNA | CAGGTGCAGCTGGTGCAGTCTGGGGCTGAGGTGAAGAAGCCTGGGGC | 1837 |
| 1981 | | CTCAGTGAAGGTTTCCTGCAAGACATCTGGATACACGTTCACCAACTA | |
| | | CTTTATGCACTGGGTGCGACAGGCCCCCGGACAAGGCCTTGAGTGGA | |
| | | TGGGAATAATCAACCCTAGTGGTGGTAGCGCAAGCTACGCACAGAAG | |
| | | TTCCAGGGCAGAATCACCATGACCAGCGACACGTCCACGAGCACAGT | |
| | | GTACATGGAACTGAGCAGCCTGAGATCTGAGGACACGGCCGTGTATT | |
| | | ATTGTGCGAGAGAGGATATTATCGTGGTGGTTCCTGCTAGGCCTCTTG | |
| | | ACTACTGGGGCCACGGAACCCTGGTCACCGTCTCCTC | |
| | LC-DNA | GAAATTGTGTTGACACAGTCTCCAGCCACCCTGTCTTTGTCTCCAGGG | 1838 |
| | | GAAAGAGCCACCCTCTCCTGCAGGGCCAGTCAGAGTTTTAGCAACTA | |
| | | CTTAGCCTGGTACCAACAGAAACCTGGCCAGGCTCCCAGGCTCCTCAT | |
| | | CTATGATGCATCCAACAGGGCCACTGGCATCCCAGCCAGGTTCAGTG | |
| | | GCAGTGGGTCTGGGACAGACTTCACTCTCACCATCAGCAGCCTAGAG | |
| | | CCTGAAGATTTTGCAGTTTATTACTGTCAGCAGCGTAGCAACTGGCCT | |
| | | CCGCTGCTCACTTTCGGCGGAGGGACCAAGGTTGAGATCAAAC | |
| S728- | HC-DNA | CAGGTGCACCTGGTGCAGTCTGGGGCTGAGATCAGGAAGCCTGGGGC | 1839 |
| 2036 | | CTCAGTGATGGTCTCCTGCAAGGCTTCTGGATACACCTTCACCGACTA | |
| | | CTATATACACTGGGTGCGACAGGCCCCTGGACAAGGGCTTGAGTGGA | |
| | | TGGGGTGGATCAACCCTAACAGTGGTGGCACAAACTATGCACAGAAA | |
| | | TTTCAGGGCAGGGTCACCATGACCAGGGACACGTCCATCAGAACAGC | |
| | | CTACATGGAACTGAGCAGGCTGAGATCTGACGACGCGGCCGTTTATT | |
| | | ACTGTGCGAGAGAAGGAATTTCAATGCTTCGGGGAGTTAGATCCTGG | |
| | | TTCGACCCCTGGGGCCAGGGAACCCTGGTCACCGTCTCCTCAG | |
| | LC-DNA | CAATCTGCCCTGACTCAGCCTGCCTCCGTGTCTGGGTCTCCTGGACAG | 1840 |
| | | TCGATCACCATCTCCTGCACTGGAACCAGCAGTGATGTTGGGAGTTAT | |
| | | AATCTTGTCTCCTGGTACCAACAGCACCCAGGCAAGGTCCCCAAACTC | |
| | | ATAATTTATGAGGTCACTAAGCGGCCCTCAGGGGTTTCTAATCGCTTC | |
| | | TCTGGCTCCAAGTCTGGCAACACGGCCTCCCTGACAATCTCTGGGCTC | |
| | | CAAACTGAGGACGAGGCTGATTATTACTGCTGCTCATATGCAGGTTTT | |
| | | AGCGCTTGGGTGTTCGGCGGAGGGACCAAACTGACCGTCCTAG | |
| S728- | HC-DNA | GAGGTGCAGCTGGTGGAGTCCGGGGGAGGCTTAGTTCAGCCTGGGGG | 1835 |
| 2111 | | GTCCCTGAGACTCTCCTGTGCAGCCTCTGGATTCACCTTCAGTAGCTA | |
| | | CTGGATGCACTGGGTCCGCCAAGCTCCAGGGAAGGGGCTGGTGTGGG | |
| | | TCTCACGTATTAATAGTGATGGGAGTAGCACAAGCTACGCGGACTCC | |
| | | GTGAAGGGCCGATTCACCATCTCCAGAGACAACGCCAAGAACACGCT | |
| | | GTATCTGCAAATGAACAGTCTGAGAGCCGAGGACACGGCTGTGTATT | |
| | | ACTGTGCAAGAGATCGGTATAGCAGCCTTGACTACTGGGGCCAGGGA | |
| | | ACCCTGGTCACCGTCTCCTCAG | |
| | LC-DNA | GAAATTGTGTTGACGCAGTCTCCAGGCACCCTGTCTTTGTCTCCAGGG | 1836 |
| | | GAAAGAGCCACCCTCTCCTGCAGGGCCAGTCAGAGTGTTAGCAGCAG | |
| | | CTACTTAGCCTGGTACCAGCAGAAACCTGGCCAGGCTCCCAGGCTCCT | |
| | | CATCTATGGTGCATCCAGCAGGGCCACTGGCATCCCAGACAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTATGGTAGCTCA | |
| | | CCCGCGCTCACTTTCGGCGGAGGGACCAAGGTGGAGATCAAAC | |
| S728- | HC-DNA | CAGGTGCAGCTGGTGGAGTCTGGGGGAGGCGTGGTCCAGCCTGGGAG | 1797 |
| 2148 | | GTCCCTGAGACTCTCCTGTGCAGCGTCTGGATTCACCTTCAGTAGCTA | |
| | | TGGCATGCACTGGGTCCGCCAGGCTCCAGGCAAGGGGCTGGAGTGGG | |
| | | TGGCAGTTATATGGTATGATGGAAGTAATAAATACTATGCAGACTCC | |
| | | GTGAAGGGCCGATTCACCATCTCCAGAGACAATTCCAAGAACACGCT | |
| | | GTATCTGCAAATGAACAGCCTGAGAGCCGAGGACACGGCTGTGTATT | |
| | | ACTGTGCGAGAGCTCCTGACTACTGGGGCCAGGGAACCCTGGTCACC | |
| | | GTCTCCTCAG | |
| | LC-DNA | GCCATCCAGATGACCCAGTCTCCATCCTCCCTGTCTGCATCTGTAGGA | 1798 |
| | | GACAGAGTCACCATCACTTGCCGGGCAAGTCAGGGCATTAGAAATGA | |
| | | TTTAGGCTGGTATCAGCAGAAACCAGGGAAAGCCCCTAAGCTCCTGA | |
| | | TCTATGCTGCATCCAGTTTACAAAGTGGGGTCCCATCAAGGTTCAGCG | |
| | | GCAGTGGATCTGGCACAGATTTCACTCTCACCATCAGCAGCCTGCAGC | |
| | | CTGAAGATTTTGCAACTTATTACTGTCTACAAGATTACAATTACCCGT | |
| | | ACACTTTTGGCCAGGGGACCAAGCTGGAGATCAAAC | |
| S728- | HC-DNA | GAGGTGCAGCTGTTGGAGTCTGGGGGAGGCTTGGTACAGCCTGGGGG | 1841 |
| 656 | | GTCCCTGAGACTCTCATGTGCAGCCTCTGGATTCACCTTTAGTAGTTA | |
| | | TGTCTTGAGCTGGGTCCGCCAGGCTCCAGGGAAGGGGCTGGAATGGG | |
| | | TCTCAGCTATTAGTGGTAGTGGTGGTATCACATATTACGCAGACTCCG | |
| | | TGAAGGGCCGCTTCACCATCTCCAGAGACAATTCCAAGAACACACTG | |
| | | TATCTGCAAATGAACAGTCTGAGAGCCGAGGACACGGCCGTATATTA | |
| | | CTGTGCGATTCGAATTACGATTTCTGGAGTGTTTACTCCCGCTTGGGA | |
| | | CTCCTGGGGCCAGGGAACCCTGGTCACCGTCTCCTCAG | |
| | LC-DNA | GACATCCAGATGACCCAGTCTCCATCCTCCCTGTCTGCATCTGTAGGA | 1842 |
| | | GACAGAGTCACCATCACTTGCCGGGCAAGTCAGAGCATTAGCACCTA | |
| | | TTTAAATTGGTATCAGCAGAAACCAGGGAAAGCCCCTAAGCTCCTGA | |
| | | TCTATGCTGCATCCAGTTTGCAAAGTGGGGTCCCATCAAGGTTCAGTG | |
| | | GCAGTGGATCTGGGACAGATTTCACTCTCACCATCAGCAGTCTGCAAC | |
| | | CTGAAGATTTTGCAACTTACTTCTGTCAACAGAGTTACAGTTCCCCAT | |
| | | TCACTTTCGGCCCTGGGACCAAAGTGGATATCAAAC | |
| S728- | HC-DNA | CAGGTGCAGCTGGTGCAGTCTGGGGCTGAGGTGAAGAAGCCTGGGGC | 1837 |
| 723 | | CTCAGTGAAGGTTTCCTGCAAGACATCTGGATACACGTTCACCAACTA | |
| | | CTTTATGCACTGGGTGCGACAGGCCCCCGGACAAGGCCTTGAGTGGA | |
| | | TGGGAATAATCAACCCTAGTGGTGGTAGCGCAAGCTACGCACAGAAG | |
| | | TTCCAGGGCAGAATCACCATGACCAGCGACACGTCCACGAGCACAGT | |
| | | GTACATGGAACTGAGCAGCCTGAGATCTGAGGACACGGCCGTGTATT | |
| | | ATTGTGCGAGAGAGGATATTATCGTGGTGGTTCCTGCTAGGCCTCTTG | |
| | | ACTACTGGGGCCACGGAACCCTGGTCACCGTCTCCTC | |
| | LC-DNA | GAAATTGTGTTGACACAGTCTCCAGCCACCCTGTCTTTGTCTCCAGGG | 1838 |
| | | GAAAGAGCCACCCTCTCCTGCAGGGCCAGTCAGAGTTTTAGCAACTA | |
| | | CTTAGCCTGGTACCAACAGAAACCTGGCCAGGCTCCCAGGCTCCTCAT | |
| | | CTATGATGCATCCAACAGGGCCACTGGCATCCCAGCCAGGTTCAGTG | |
| | | GCAGTGGGTCTGGGACAGACTTCACTCTCACCATCAGCAGCCTAGAG | |
| | | CCTGAAGATTTTGCAGTTTATTACTGTCAGCAGCGTAGCAACTGGCCT | |
| | | CCGCTGCTCACTTTCGGCGGAGGGACCAAGGTTGAGATCAAAC | |
| S728- | HC-DNA | GAGGTGCAGCTGGTGGAGTCTGGGGGAGGCTTGGTCCAGCCTGGGGG | 1803 |
| 826 | | GTCCCTGAGACTCTCCTGTGCAGCCTCTGGATTCACCTTTGGTAGCTA | |
| | | TTGGATGAACTGGGTCCGCCAGGCTCCAGGGAAGGGGCTGGAGTGGG | |
| | | TGGCCAACATAAACGAAGATGGAAGTGAGAAATACTATGTGGACTCT | |
| | | GTGAAGGGCCGATTCACCATCTCCAGAGACAACGCCAAGAACTCACT | |
| | | ATATCTGCAAATGAACAGCCTGAGAGCCGAGGACACGGCTGTGTATT | |
| | | ACTGTGCGAGAGGACATTCTCTGGGTGAGTGGGGCCAGGGATCCCCG | |
| | | GTCACCGTCTCCTCAG | |
| | LC-DNA | TCTTCTGAGCTGACTCAGGACCCTGCTGTGTCTGTGGCCTTGGGACAG | 1804 |
| | | ACAGTCAGGATCACATGCCAAGGAGACAGCCTCAGAAGCTATTCTGC | |
| | | AAGCTGGTACCAGCAGAAGCCAGGACAGGCCCCTGTACTTGTCATCT | |
| | | ATATTAAAAACAAACGACCCTCAGGGATCCCAGACCGATTCTCTGGC | |
| | | TCCAGCTCAGGAAACACAGCTTCCTTGACCATCACTGGGGCTCAGGC | |
| | | GGAAGATGAGGCTGACTATTACTGTAACTCCCGGGACAGCAGTGGTG | |
| | | ACCATCTGGTGTTCGGCGGAGGGACCAAGCTGACCGTCCTAG | |
| S728- | HC-DNA | CAGGTGCACCTGGTGCAGTCTGGGGCTGAGATCAGGAAGCCTGGGGC | 1839 |
| 959 | | CTCAGTGATGGTCTCCTGCAAGGCTTCTGGATACACCTTCACCGACTA | |
| | | CTATATACACTGGGTGCGACAGGCCCCTGGACAAGGGCTTGAGTGGA | |
| | | TGGGGTGGATCAACCCTAACAGTGGTGGCACAAACTATGCACAGAAA | |
| | | TTTCAGGGCAGGGTCACCATGACCAGGGACACGTCCATCAGAACAGC | |
| | | CTACATGGAACTGAGCAGGCTGAGATCTGACGACGCGGCCGTTTATT | |
| | | ACTGTGCGAGAGAAGGAATTTCAATGCTTCGGGGAGTTAGATCCTGG | |
| | | TTCGACCCCTGGGGCCAGGGAACCCTGGTCACCGTCTCCTCAG | |
| | LC-DNA | CAATCTGCCCTGACTCAGCCTGCCTCCGTGTCTGGGTCTCCTGGACAG | 1840 |
| | | TCGATCACCATCTCCTGCACTGGAACCAGCAGTGATGTTGGGAGTTAT | |
| | | AATCTTGTCTCCTGGTACCAACAGCACCCAGGCAAGGTCCCCAAACTC | |
| | | ATAATTTATGAGGTCACTAAGCGGCCCTCAGGGGTTTCTAATCGCTTC | |
| | | TCTGGCTCCAAGTCTGGCAACACGGCCTCCCTGACAATCTCTGGGCTC | |
| | | CAAACTGAGGACGAGGCTGATTATTACTGCTGCTCATATGCAGGTTTT | |
| | | AGCGCTTGGGTGTTCGGCGGAGGGACCAAACTGACCGTCCTAG | |
| S210- | HC-DNA | CAGGTGCAGCTGGTGCAGTCTGGGGCTGAGGTGAAGAAGCCTGGGGC | 1843 |
| 530 | | CTCAGTGAAGGTCTCCTGCAAGGCTTCTGGATACACCTTCACCGGCTA | |
| | | CTTTATTCACTGGGTGCGACAGGCCCCTGGACAAGGGCTTGAATATAT | |
| | | GGGATGGATCAACCCTAATAGTGCTGGCACAAACTATGCACAGAAGT | |
| | | TTCAGGGCAGGGTCACCATGACCGGGGACACGTCCATCAGCACAGTC | |
| | | TACATGGAGCTGAGCAGGCTGAGATCTGACGACACGGCCATGTATTA | |
| | | CTGTGCGAGAGTATTTTTTGACTGGTTATTGCCGTTTGACTACTGGGG | |
| | | CCAGGGAACCCTGGTCACCGTCTCCTCAG | |
| | LC-DNA | CAGTCTGCCCTGACTCAGCCTGCCTCCGTGTCTGGGTCTCCTGGACAG | 1844 |
| | | TCGATCACCATCTCCTGCACTGGAACCAGCAGTGATGTTGGGAGTTAT | |
| | | AACCTTGTCTCCTGGTATCAACAGCACCCAGGCAAAGCCCCCAAACT | |
| | | CATGATTTATGAGGTCAGTAAGCGGCCCTCAGGGGTTTCTAATCGCTT | |
| | | CTCTGGCTCCAAGTCTGGCAACACGGCCTCCCTGACAATCTCTGGGCT | |
| | | CCAGGCTGAGGACGAGGCTGATTATTACTGCTGCTCATATGCAGGTA | |
| | | GTAATTATGTCTTCGGAACTGGGACCAAGGTCACCGTCCTAG | |
| S210- | HC-DNA | GAGGTGCAGCTGTTGGAGTCTGGGGGAGGCTTGGTACAGCCTGGGGG | 1845 |
| 1129 | | GTCCCTGAGACTCTCCTGTGTAGCCTCTAGATTCACCTTTAGCGACTA | |
| | | CGCCATGAGCTGGGTCCGCCAGCCTCCAGGGAAGGGGCTGGAGTGGG | |
| | | TCTCAAGTATTAGTGGTAGTGGTGGTATTACTTACTACGCAGACTCCG | |
| | | TGAAGGGCCGGTTCACCATCTCCAGAGACAATTCCAAGAACACACTG | |
| | | TATCTGCAAATAAAGAGCCTGAGAGCCGAGGACACGGCCATATATTA | |
| | | CTGTGCGAAGGAACGATCTAACTGGAACTACGTGGAAAACTTTGACT | |
| | | ACTGGGGCCAGGGAACCCTGGTCACCGTCTCCTCAG | |
| | LC-DNA | CAGACTGTGGTGACTCAGGAGCCCTCACTGACTGTGTCCCCAGGAGG | 1846 |
| | | GACAGTCACTCTCACCTGTGCTTCCAGTACTGGAACAGTCACCAGTGC | |
| | | TTTCTTTCCAAACTGGTTCCAGCAGAAACCTGGACAAGCACCCAGGG | |
| | | CACTGATTTATAGTACAACCAACAAATACTCCTGGACCCCTGCCCGGT | |
| | | TCTCAGGCTCCCTCCTTGGGGGCAAAGCTGCCCTGACACTGTCAGGTG | |
| | | TGCAGCCTGAGGACGAGGCTGACTATTACTGCCTGCTCTTCTATGGTG | |
| | | GTGCTCGGCCCCATGTGGTTTTCGGCGGAGGGACCAAGCTGACCGTC | |
| | | CTAG | |
| S451- | HC-DNA | CAGGTGCAGCTACAGCAGTGGGGCGCGGGACTGTTGAAGCCTTCGGA | 1847 |
| 5 | | GACCCTGTCCCTCACCTGCGCTGTCTATGGTGCGTCCGTCAGTGGTTA | |
| | | CTTCTGGAGCTGGATCCGCCAGCCCCCAGGGAAGGGGCTGGAGTGGA | |
| | | TTGGAGAAATCAATCGTTTTGGAAGCACCAACTACAACCCGTCCCTCA | |
| | | AGAGTCGAGTCACCTTATCAGTGGACACGTCCAGGAACCAGTTCTCC | |
| | | CTGAAGCTGGGCTCTGTGACCGCCGCGGACACGGCAATGTATTACTG | |
| | | TGCGAGAGGCAGTCAGGCCAACCCCCTCGTACGATTTTTTGACAGCCC | |
| | | CGTCACGGCTTTTGATATCTGGGGCCAAGGGACAATGGTCACCGTCTC | |
| | | TTCAG | |
| | LC-DNA | GAAATAGTGATGACGCAGTCTCCAGCCACCCTGTCTGTGTCTCCAGGG | 1848 |
| | | GAAAGGGCCACCCTCTCCTGCAGGGCCAGTCAGAGTATTAAGAGCAA | |
| | | CTTAGCCTGGTACCAACAAAAACCTGGCCAGGCTCCCAGGCTCCTCAT | |
| | | CTATGGTGCATCCACCAGGGCCACTGGTATCCCAGCCAGGTTCAGTG | |
| | | GCAGTGGGTCTGGGACAGAGTTCACTCTCACCATCAGCCGCCTGCAG | |
| | | TCTGAAGATTTTGCACTTTATTACTGTCAGCAGTATGATAACTGGCCT | |
| | | CCGTACACTTTTGGCCAGGGGACCAAGCTGGAGATCAAAC | |
| S451- | HC-DNA | CAGATCACCTTGAAGGAGTCTGGTCCTACGCTGGTGAAACCCACACA | 1849 |
| 506 | | GACCCTCACGCTGACCTGCACCTTCTCTGGGTTCTCATTCACCAGTAG | |
| | | TGGAGTGGGTGTGGGCTGGATCCGTCAGCCCCCAGGAAAGGCCATGG | |
| | | AGTGGCTTGCACTCATTTATTGGGATGATGATAAGCGTTACAGCCCAT | |
| | | CTCTGAAGAGCAGGCTCACCATCACCAAGGACACCTCCAAAAACCAG | |
| | | GTGGTCCTTAAAATGACCAATATGGACCCTGTGGACACAGCCACATA | |
| | | TTACTGTGCACGCCATACAGTGGCTACGATTGTTGACTACTGGGGCCA | |
| | | GGGAACCCTGGTCACCGTCTCCTCAG | |
| | LC-DNA | CAGTCTGCCCTGACTCAGCCTGCCTCCGTGTCTGGGTCTCCAGGACAG | 1850 |
| | | TCGATCACCATCTCCTGCACTGGAACCAGCAGTGACGTTGGTGGTTAT | |
| | | AACTATGTCTCCTGGTACCAACAACACCCAGGCAAAGCCCCCAAACT | |
| | | CATGATTTATGAGGTCAGTAATCGGCCCTCAGGGGTCCCTGATCGCTT | |
| | | CTCTGGCTCCAAGTCTGGCAACACGGCCTCCCTGACCATCTCTGGGCT | |
| | | CCAGGCTGAGGACGAGGCTGATTATTACTGCGGCTCATATACAACCA | |
| | | GCAGCACTCCTGTGGTTTTCGGCGGAGGGACCAAGCTGACCGTCCTA | |
| | | G | |
| S451- | HC-DNA | GAGGTGCAGCTGGTGGAGACTGGAGGAGGCTTGATCCAGCCTGGGGG | 1851 |
| 1140 | | GTCCCTGAGACTCTCCTGTGCAGCCTCTGGGATCACCGTCAGTAGCAA | |
| | | CTACATGAACTGGGTCCGCCTGGCTCCAGGGAAGGGGCTGGAGTGGG | |
| | | TCTCACTCATTTATAGCGGTGGTAGCACATTCTACGCAGACTCCGTGA | |
| | | AGGGCCGATTCACCATCTCCAGAGACAATTCCAAGAACACGCTGTAT | |
| | | CTTCAAATGAACAGCCTGAGAGCCGAGGACACGGCCGTGTATTATTG | |
| | | TGCGAGAGAAGGTTTAGTGGGAGCTACGACGGCTTTTGACTACTGGG | |
| | | GCCAGGGAACGCTGGTCACCGTCTCCTCAG | |
| | LC-DNA | GACATCCAGTTGACCCAGTCTCCATCCTTCCTGTCTGCATCTGTAGGA | 1852 |
| | | GACAGAGTCACCATCACTTGCCGGGCCAGTCAGGGCATTAGCAGTTA | |
| | | TTTAGCCTGGTATCAGCTACAACCAGGGAAAGCCCCTAAGCTCCTGAT | |
| | | CTATGCTGCATCCACTTTGCAAAGTGGGGTCCCATCAAGGTTCAGCGG | |
| | | CAGTGGATCTGGGACAGAATTCACTCTCACAATCAGCAGCCTGCAGC | |
| | | CTGAAGATTTTGCAACTTATTACTGTCAACAGCTTAATGGTCACCCCC | |
| | | AGGGGACGTTCGGCCAAGGGACCAAGGTGGAAATCAAAC | |
| S451- | HC-DNA | CAGGTCACCTTGAGGGAGTCTGGTCCTGCGCTGGTGAAACCCACACA | 1853 |
| 1190 | | GACCCTCACACTGACCTGCACCTTCTCTGGGTTCTCACTCACCACTAG | |
| | | TGGAATGTGTGTGAGCTGGATCCGTCAGCCCCCAGGGAAGGCCCTGG | |
| | | AGTGGCTTGCACGCATTGATTGGGATGATGATAAATACTACAGCACA | |
| | | TCTCTGAAGGCCAGGCTCACCATCTCCAAGGACACCTCCAAAAACCA | |
| | | GGTGGTCCTTACAATGACCAACATGGACCCTGTGGACACAGCCACGT | |
| | | ATTACTGTGCACGGACTTCAGTGGGAGGTACCAAGTACTACTTTGACT | |
| | | ACTGGGGCCAGGGAACGCTGGTCACCGTCTCCTCAG | |
| | LC-DNA | CAGTCTGTGCTGACTCAGCCACCCTCAGCGTCTGGGACCCCCGGGCA | 1854 |
| | | GAGGGTCACCATCTCTTGTTCTGGAAGCAGCTCCAACATCGGAAGAA | |
| | | ATACTGTAAACTGGTACCAGCAGCTCCCAGGAACGGCCCCCAAACTC | |
| | | CTCATCTATAGTAATAATCAGCGGCCCTCAGGGGTCCCTGACCGATTC | |
| | | TCTGGCTCCAAGTCTGGCACCTCAGCCTCCCTGGCCATCAGTGGGCTC | |
| | | CAGTCTGAGGATGAGGCTGATTATTACTGTGCAGCATGGGATGACAG | |
| | | CCTGAATGGGGGGGTATTCGGCGGAGGGACCAAGCTGACCGTCCTAG | |
| S626- | HC-DNA | CAGCTGCAGCTGCAGGAGTCGGGCCCAGGACTGGTGAAGCCTTCGGA | 1855 |
| 84 | | GGCCCTGTCCCTCACCTGCACTGTCTCTGGTGGCTCCATCAGCACTAG | |
| | | TAATTACTACTGGGGCTGGATCCGCCAGCCCCCAGGGAAGGGGCTGG | |
| | | AGTGGATTGGGAGTATCTATTATCGTGGGGGCACCCACTACAACCCG | |
| | | TCCCTCAAGACTCGAGTCACCATATCCGTAGACACGTCCAAGAACCA | |
| | | GTTCTCCCTGAAGCTGAGCTCTGTGACCGCCGCAGACACGGCTGTGTA | |
| | | TTACTGTGCGAGACATACCTATTTCTATGATATCGTGGGGGCAGCGGT | |
| | | TTGGGAACCTTTTGATATCTGGGGCCAAGGGACAATGGTCACCGTCTC | |
| | | TTCAG | |
| | LC-DNA | GAAATTGTGTTGACGCAGTCTCCAGGCACCCTGTCTTTGTCTCCAGGG | 1856 |
| | | GAAAGAGCCACCCTCTCCTGCAGGGCCAGTCAGAGTGTTAGCAGCAG | |
| | | CTACTTAGCCTGGTACCAGCAGAAACCTGGCCAGGCTCCCAGGCTCCT | |
| | | CATCTCTGATGCATCCAGCAGGGCCACTGGCATCCCAGACAGGTTCA | |
| | | GTGGCAGTGGGTCTGGGACAGACTTCACTCTCACCATCAGCAGACTG | |
| | | GAGCCTGAAGATTTTGCAGTGTATTACTGTCAGCAGTATGGTAGCTCA | |
| | | CCTCCGTGGACGTTCGGCCAAGGGACCAAGGTGGAAATCAAAC | |
| S626- | HC-DNA | CAGCTGCAGCTGCAGGAGTCGGGCCCAGGACTGGTGAAGCCTTCGGA | 1857 |
| 161 | | GACCCTGTCCCTCACCTGCACTGTCTCTGGTGGCTCCATCAGCAGTAA | |
| | | TAATTACTACTGGGGCTGGATCCGCCAGCCCCCAGGGAAGGGGCTGG | |
| | | AGTGGATTGGGAGTATCTATTATAGTGGCAGCACCTACTACAACCCGT | |
| | | CTCTCAAGAGTCGAGTCACCATGTCCGTAGACACGTCCAAGAACCAG | |
| | | TTCTCCCTGAAGCTGAGCTCTGTGACCGCCGCAGACACGGCTGTGTAT | |
| | | CACTGTGCGAGACAAGGACCGAATTACTATGATAGAAGTGGTTATTA | |
| | | TTACGTCGGCCCTTTTGATATCTGGGGCCAAGGGACAATGGTCACCGT | |
| | | CTCTTCAG | |
| | LC-DNA | CAGTCTGTGTTGACGCAGCCGCCCTCAGTGTCTGCGGCCCCAGGACA | 1858 |
| | | GAAGGTCACCATCTCCTGCTCTGGAAGCAGCTCCAACATTGGGAATA | |
| | | ATTCTGTATCCTGGTACCAGCACCTCCCAGGAACAGCCCCCAAACTCC | |
| | | TCATCTATGAAAATAATGAGCGACCCTCAGGGATTCCTGACCGATTCT | |
| | | CTGGCTCCAAGTCTGGCACGTCAGCCACCCTGGGCATCACCGGACTCC | |
| | | AGACTGGGGACGAGGCCGATTATTACTGCGAAACATGGGATAGGAGC | |
| | | CTGAGTGCTTCCTTCGGAACTGGGACCAAGGTCACCGTCCTAG | |
| S626- | HC-DNA | CAGGTGCAGCTGGTGCAGTCTGGGGCTGAGGTGAAGAAGCCTGGGGC | 1859 |
| 664 | | CTCAGTGAAGGTCTCCTGCAGGGTTTCTGGATACACCTTCACCGGCTA | |
| | | CTATATACACTGGGTGCGGCAGGCCCCTGGACAAGGGCTTGAGTGGA | |
| | | TGGGATGGATCAACCCTAACAGTGGTGGCACAAACTATGCACAGAAG | |
| | | TTTCAGGGCAGGGTCACCATGACCAGGGACACGTCCATCACCACAGC | |
| | | CTACATGGAGCTGAGCAGGCTGAGATCTGACGACACGGCCGTGTATT | |
| | | ACTGTGCGAGAGTCCCTATGATCCTAGTGGTTGATCATTGGGGTTCCT | |
| | | ACTTTGACTACTGGGGCCAGGGAACCCTGGTCACCGTCTCCTCAG | |
| | LC-DNA | CAGTCTGCCCTGACTCAGCCTGCCTCCGTGTCTGGGTCTCCTGGACAG | 1860 |
| | | TCGATCACCATCTCCTGCACTGGAACCAGCAGTGATGTTGGTGGTTAT | |
| | | AACTATGTCTCCTGGTACCAACAATACCCAGGCAAAGCCCCCAAACT | |
| | | CATGATTTATGATGTCAGTAAGCGGCCCTCAGGGGTTTCTAATCGCTT | |
| | | CTCTGGCTCCAAGTCTGGCAACACGGCCTCCCTGACAATCTCTGGGCT | |
| | | CCAGGCAGAGGACGAGGCTGATTATTACTGCTGCTCATATGCAGGTA | |
| | | GTAGCGCTTTAGTATTCGGCGGAGGGACCAAGCTGACCGTCCTAG | |
| S728- | HC-DNA | CAGATCACCTTGAAGGAGTCTGGTCCTACGCTGGTGAAACCCACACA | 1861 |
| 209 | | GACCCTCACACTGACCTGCACCCTCTCTGGATTCTCACTCAGCACTAG | |
| | | TGGAGTTAGTGTGGGCTGGATCCGTCAGCCCCCAGGAAAGGCCCTGG | |
| | | AATGGCTTGCAGTCATTTTTTGGGATGATGATAAGCGCTACAACCCAT | |
| | | CTCTGAAGAGCAGGCTCACCATCGCCAAGGACACCTCCAAAAGCCAG | |
| | | GTGGTCCTTACAATGACCAACCTGGACCCTGTGGACACTGGCACATAT | |
| | | TACTGTGTGTCGGGCAGCTCGTATTACTACTACTACTACATGGACGTC | |
| | | TGGGGCAAAGGGACCACGGTCACCGTCTCCTCA | |
| | LC-DNA | GACATCCAGATGACCCAGTCTCCATCTTCCGTGTCTGCATCTATTGGA | 1862 |
| | | GACAGAGTCACCATCACTTGTCGGGCGAGTCAGGATATTAGCACCTG | |
| | | GTTAGCCTGGTATCAGCAAAAACCAGGGAGAGCCCCTAACCTCCTGA | |
| | | TCTATGGTGCATCCAGCTTGCAAAGTGGGGTCCCATCAAGGTTCAGCG | |
| | | GCAGTGGATCTGGGACAGATTTCACTCTCACCATCAGCAGCCTGCAG | |
| | | CCTGAAGATTTTGCAACTTATTATTGTCAACAGGCTACCAGTTTCCCT | |
| | | CTCACTTTCGGCGGAGGGACCAAGGTCGAGATCAAAC | |
| S728- | HC-DNA | CAGGTGCAGCTGCAGGAGTCGGGCCCAGGACTGGTGAAGCCTTCACA | 1863 |
| 369 | | GACCCTGTCTCTCACCTGCTCTGTCTCTGGTGGCTCCATCAGCAGTGG | |
| | | TGGTTACTACTGGAGCTGGATCCGCCAGCACCCAGGGAAGGGCCTGG | |
| | | AGTGGATTGGGTACATGTATTACAGTGGGAGCACTTATTATAACCCGT | |
| | | CCCTCAAGAGTCGAGTTACCATATTCGTGGACACGTCTAAGAACCACT | |
| | | TCTCCCTGAAACTGACCTCTGTTACTGCCGCGGACACGGCCGTTTATT | |
| | | ACTGTGCGAGAGATTCGTACGAAAATTACTATGGTTCGGGGAGCCTG | |
| | | GAGCCCAACTACCACCACTACAATATGGACGTCTGGGGCCAAGGGAC | |
| | | CACGGTCACCGTCTCCTCA | |
| | LC-DNA | GACGTCCAGATGACCCAGTCTCCTTCCACCCTGTCTGCATCTATAGGA | 1864 |
| | | GACAGAGTCACCATCACTTGCCGGGCCAGTCAGAGTATTAGTGGCTG | |
| | | GTTGGCCTGGTATCAGCAGAGACCAGGGAAAGCCCCTAAACTCCTGA | |
| | | TCTATAGGGCGTCTAGTTTAGATTTTGGGGTCCCATCAAGGTTCAGCG | |
| | | GCAATGGATCTGGGACAGAATTCACTCTCACCATCAGCAGCCTGCAG | |
| | | CCTGATGATTTTGCAACTTATTACTGCCAACAGTATCATACTTATCGG | |
| | | ACGTTCGGCCAAGGGACCAAGGTGGAAGTCAAAC | |
| S728- | HC-DNA | GAGGTGCAGCTGGTGGAGTCTGGAGGAGGCTTGATCCAGCCTGGGGG | 1865 |
| 430 | | GTCCCTGAGACTCTCCTGTGCAGCCTCTGGGTTCACCGTCAGTAGCAA | |
| | | CTACATGAGCTGGGTCCGCCAGGCTCCAGGGAAGGGGCTGGAGTGGG | |
| | | TCTCAGTTATTTATAGCGGTGGTAGTACATACTACGCAGACTCCGTGA | |
| | | AGGGCCGATTCACCATCTCCAGAGACAATTCCAAGAACACGCTGTAT | |
| | | CTTCAAATGAACAGCCTGAGAGCCGAGGACACGGCCGTGTATTACTG | |
| | | TGCGAGAACCCCGAGGGGCAGCAGGCGGGGGGCTTTTGATATCTGGG | |
| | | GCCAAGGGACAATGGTCACCGTCTCTTCAG | |
| | LC-DNA | GACATCCAGATGACCCAGTCTCCATCCTCCCTGTCTGCATCTGTAGGA | 1866 |
| | | GACAGAGTCACCATCACTTGCCAGGCGAGTCAGGACATTAGCGACTA | |
| | | TTTAAATTGGTATCAGCAGAAACCAGGGAAAGCCCCTAAGCTCCTGA | |
| | | TCTACGATGCATCCAATTTGGAAACAGGGGTCCCATCAAGGTTCAGT | |
| | | GGAAGTGGATCTGGGACAGATTTTACTTTCACCATCAGCAGCCTGCA | |
| | | GCCTGAAGATATTGCAACATATTACTGTCAACAGTATGATAATCTCCC | |
| | | TCCGCTCACTTTCGGCGGAGGGACCAAGGTGGAGATCAAAC | |
| S728- | HC-DNA | CAGGTGCAGCTGGTGCAGTCTGGGGCTGAGGTGAAGAAGCCTGGGGC | 1867 |
| 537 | | CTCAGTGAAAGTCTCCTGTAAGACTTCTGGATACACCTTCACCGGCTT | |
| | | CTATTTGCACTGGCTGCGACAGGCCCCTGGACAAGGGCTTGAGTGGA | |
| | | TGGGACGAATCAACCCTAACACCGGTGACACAGACTATGCACAGAAG | |
| | | TTTCAGGGCAGGGTCACCATGACCAGGGACACCTCCATCAGCACAGC | |
| | | CTACATGGAACTGAGCAGGCTGAGAGCTGACGACACGGCCGTGTATT | |
| | | ATTGTGCGAGAACGCCCGGGCAAACACGACAACTGTTCGTGGGGACT | |
| | | AATGTTCTTGATGTCTGGGGCCAAGGGACAATGGTCACCGTCTCTTCA | |
| | | G | |
| | LC-DNA | GACATCCAGATGACCCAGTCTCCATCTTCCGTGTCTGCATCTGTAGGG | 1868 |
| | | GACAGAGTCACCATCACTTGTCGGGCGAGTCAGGGTATTAGCAGCTG | |
| | | GTTAGCCTGGTATCAACAGAAACCAGGGAAAGCCCCTAAGGTCCTCA | |
| | | TTTTTGCTGCATCCAGTTTGCAAAGTGGGGTCCCATCAAGGTTCAGCG | |
| | | GCAGTGGATCTGGGACAGATTTCACTCTCACCATCACCAGCCTGCAGC | |
| | | CTGAAGATTTTGCAACTTACTTTTGTCAACAGACTAACAGTTTCCCTC | |
| | | CCACTTTCGGCGGAGGGACCAAGGTGGAGATCAAAC | |
| S728- | HC-DNA | GAGGTGCAACTGGTGGAGTCTGGGGGAGGCTTGGTCCAGCCTGGGGG | 1869 |
| 1157 | | GTCCCTGAGACTCTCCTGTGCAGCCTCTGGATTACTCGTCAGTAGAAA | |
| | | TTACATGAACTGGGTCCGCCAGGCTCCAGGGAAGGGGCTGGAATGGG | |
| | | TCTCAATTATTTATAGTGGTGGCAGCACATTCTACGCAGACTCCGTGG | |
| | | AGGGCCGATTCACCATCTCCAGAGACGAGTCCAAGAACACACTGTAT | |
| | | CTTCAAATGAACAGTCTGAGAACTGACGACACGGCTGTGTATTACTGT | |
| | | GCGAGAGATCTCTCCGACTACGGTGGGATTGACTGCTGGGGCCAGGG | |
| | | AACCCTGGTCACCGTCTCCTCAG | |
| | LC-DNA | CCTATGAACTGACTCAGCCACTCTCAGTGTCAATGGCCCTGGGACAG | 1870 |
| | | ACGGCCAGGATTTCCTGTGGGGGAGACAACGTGGGAAGTCAAAATGT | |
| | | GCACTGGTACCAGCAGAGGCCAGGCCAGGCCCCTGTGCTGGTCATCT | |
| | | ATAGGGATAGCAACCGGCCCTCTGGGATCCCTGAGCGATTCTCTGGCT | |
| | | CCAAGTCGGGGAACACGGCCACCCTGACCATCAGCAGAGCCCAAGCC | |
| | | GGGGATGAGGCTGACTATTACTGTCAGGTGTGGGACAGCAGCACTGT | |
| | | GGCTTTCGGCGGAGGGACCAAGCTGACCGTCCTAG | |
| S728- | HC-DNA | CAGGTGCAGCTGCAGGAGTCGGGCCCAGGACTGGTGAAGCCTTCGGG | 1871 |
| 1261 | | GACCCTGTCCCTCACCTGCGCTGTCTCTGGTGGCTCCATCAGTAATAA | |
| | | TAACTGGTGGATTTGGGTCCGCCAGCCCCCAGGGAAGGGGCTGGAGT | |
| | | GGATTGGGGAAATCCATCATAGTGGGAGCACCGACTACAACCCGTCC | |
| | | CTCAAGAGTCGAGTCACCATATCAATAGACAAGTCCAAGAACCAGTT | |
| | | CTCCCTGAGGCTGAGCTCTGTGACCGCCGCGGACACGGCCGTGTATTA | |
| | | CTGTGCGAGAAAGCCAGAACCGTACTACTACTACTACTACATGGACG | |
| | | TCTGGGGCAAAGGGACCACGGTCACCGTCTCCTCA | |
| | LC-DNA | GAGACTGTGTTGACACAGTCTCCAGGCACCCTGTCTTTGTCTCCGGGG | 1872 |
| | | GAAAGAGCCACCCTCTCCTGCAGGGCCAGTCAGAGTGTTAGCAGCAG | |
| | | TTATATAACCTGGTACCAGCAGAAACCTGGCCAGGCTCCCAGGCTCCT | |
| | | CATCTATGGTGCATCCAGCAGGGCCACTGGCATCCCAGACAGGTTCA | |
| | | GTGGCAGTGGGTCTGGGACAGACTTCACTCTCACCATCAGTAGACTG | |
| | | GAGCCTGAAGATTTCGCAGTGTATTACTGTCAGCAATATCGTAGCCCC | |
| | | TGGGGGCTCACTTTCGGCCCTGGGACCAAAGTGGATATCAAAC | |
| S728- | HC-DNA | CAGGTCCAGCTGGTGCAGTCTGGGGCTGAGGTGGTGAAGCCTGGGTC | 1873 |
| 1690 | | CTCGGTGAAGGTCTCCTGCAAGGCCTCTGGAGGCACCTTCACCAGGT | |
| | | ACGCTATTAGCTGGGTGCGACAGGCCCCCGGACAAGGGCCTGAGTGG | |
| | | ATGGGAAGGATCATCCCTATGTTTGGAATAGCAAACTACGCACAGAG | |
| | | GTTCCAGGGCAGAGTCACGATGACCGCGGACAAATCCACGAGCACTG | |
| | | CCTACATGGAGCTGAGCAGCCTGAGATCTGAGGACACGGCCGTCTAT | |
| | | TACTGTGCGACATGCCAGTATTATTATGACAGTAGTGGTTATGGGTCC | |
| | | CTTGACTACTGGGGCCAGGGAACCCAGGTCACCGTCTCCTCAG | |
| | LC-DNA | GAAATTGTGTTGACGCAGTCTCCAGGCACCCTGTCTTTGTCTCCAGGG | 1874 |
| | | GAAAGAGTCACCCTCTCCTGCAGGGCCAGTCAGAGTATTAGCAGCAA | |
| | | CTTCTTAGCCTGGTACCAGCAGAAACCTGGCCAGGCTCCCAGGCTCCT | |
| | | CATCTCTGGTGCATCCAGCAGGGCCACTGGCATCCCAGACAGGTTCA | |
| | | GTGGCGGTGGGTCTGGGACAGACTTCACTCTCACCATCAGCAGACTG | |
| | | GAGCCTGAAGATTTTGCAGTGTATTACTGTCAGCAGTATCATAGCTCA | |
| | | CCGCGGACGTTCGGCCAAGGGACCAAGGTGGAAATCAAAC | |

TABLE 3
Summary of SEQ ID NOS.

| | | HC | | | | | | | | | LC | | | | | | | | | |
|---|---|---|---|---|---|---|---|---|---|---|---|---|---|---|---|---|---|---|---|---|
| | | vari- | HC | HCD | HC | HFR | HFR | HFR | HFR | | vari- | LCD | LCD | LCD | LFR | LFR | LFR | LFR | HC- | LC- |
| Clone | HC | able | DR1 | R2 | DR3 | 1 | 2 | 3 | 4 | LC | able | R1 | R2 | R3 | 1 | 2 | 3 | 4 | DNA | DNA |
| S20- | 1 | 2 | 3 | 4 | 5 | 6 | 7 | 8 | 9 | 10 | 11 | 12 | 13 | 14 | 15 | 16 | 17 | 18 | 1603 | 1604 |
| 15 | | | | | | | | | | | | | | | | | | | | |
| S20- | 19 | 20 | 21 | 22 | 23 | 24 | 25 | 26 | 9 | 27 | 28 | 29 | 30 | 31 | 32 | 33 | 34 | 35 | 1605 | 1606 |
| 22 | | | | | | | | | | | | | | | | | | | | |
| S20- | 36 | 37 | 38 | 39 | 40 | 41 | 42 | 43 | 44 | 45 | 46 | 47 | 48 | 49 | 50 | 51 | 52 | 53 | 1607 | 1608 |
| 31 | | | | | | | | | | | | | | | | | | | | |
| S20- | 54 | 55 | 56 | 57 | 58 | 24 | 25 | 59 | 60 | 61 | 62 | 63 | 64 | 65 | 66 | 67 | 68 | 69 | 1609 | 1610 |
| 40 | | | | | | | | | | | | | | | | | | | | |
| S20- | 70 | 71 | 72 | 73 | 74 | 75 | 7 | 76 | 60 | 77 | 78 | 79 | 80 | 81 | 82 | 83 | 84 | 85 | 1611 | 1612 |
| 58 | | | | | | | | | | | | | | | | | | | | |
| S20- | 86 | 87 | 3 | 88 | 89 | 24 | 90 | 91 | 44 | 92 | 93 | 63 | 94 | 95 | 96 | 67 | 97 | 69 | 1613 | 1614 |
| 74 | | | | | | | | | | | | | | | | | | | | |
| S20- | 98 | 99 | 100 | 101 | 102 | 103 | 104 | 105 | 106 | 107 | 108 | 63 | 64 | 65 | 66 | 67 | 68 | 18 | 1615 | 1616 |
| 86 | | | | | | | | | | | | | | | | | | | | |
| S24- | 109 | 110 | 56 | 111 | 112 | 113 | 114 | 115 | 60 | 116 | 117 | 118 | 119 | 120 | 121 | 122 | 123 | 69 | 1617 | 1618 |
| 68 | | | | | | | | | | | | | | | | | | | | |
| S24- | 124 | 125 | 126 | 127 | 128 | 129 | 130 | 131 | 60 | 132 | 133 | 134 | 135 | 136 | 50 | 137 | 138 | 53 | 1619 | 1620 |
| 105 | | | | | | | | | | | | | | | | | | | | |
| S24- | 139 | 140 | 141 | 142 | 143 | 144 | 145 | 146 | 147 | 148 | 149 | 150 | 151 | 152 | 66 | 153 | 68 | 18 | 1621 | 1622 |
| 178 | | | | | | | | | | | | | | | | | | | | |
| S24- | 154 | 155 | 156 | 157 | 158 | 159 | 160 | 161 | 147 | 162 | 163 | 63 | 164 | 165 | 66 | 67 | 166 | 167 | 1623 | 1624 |
| 188 | | | | | | | | | | | | | | | | | | | | |
| S24- | 168 | 169 | 170 | 171 | 172 | 173 | 174 | 175 | 147 | 176 | 177 | 178 | 179 | 180 | 181 | 182 | 183 | 184 | 1625 | 1626 |
| 202 | | | | | | | | | | | | | | | | | | | | |
| S24- | 185 | 186 | 187 | 188 | 189 | 190 | 160 | 191 | 147 | 192 | 193 | 194 | 135 | 195 | 50 | 182 | 196 | 85 | 1627 | 1628 |
| 278 | | | | | | | | | | | | | | | | | | | | |
| S24- | 197 | 198 | 199 | 200 | 201 | 202 | 203 | 204 | 60 | 205 | 206 | 207 | 208 | 209 | 210 | 182 | 211 | 53 | 1629 | 1630 |
| 339 | | | | | | | | | | | | | | | | | | | | |
| S24- | 212 | 213 | 214 | 215 | 216 | 217 | 218 | 219 | 220 | 221 | 222 | 223 | 224 | 225 | 226 | 227 | 228 | 69 | 1631 | 1632 |
| 472 | | | | | | | | | | | | | | | | | | | | |
| S24- | 229 | 230 | 231 | 232 | 233 | 190 | 160 | 234 | 60 | 235 | 236 | 237 | 135 | 238 | 50 | 239 | 196 | 85 | 1633 | 1634 |
| 490 | | | | | | | | | | | | | | | | | | | | |
| S24- | 240 | 241 | 242 | 243 | 244 | 245 | 7 | 115 | 9 | 246 | 247 | 248 | 249 | 250 | 251 | 252 | 253 | 85 | 1635 | 1636 |
| 494 | | | | | | | | | | | | | | | | | | | | |
| S24- | 254 | 255 | 199 | 256 | 257 | 258 | 203 | 259 | 60 | 260 | 261 | 262 | 263 | 264 | 265 | 266 | 267 | 53 | 1637 | 1638 |
| 566 | | | | | | | | | | | | | | | | | | | | |
| S24- | 268 | 269 | 270 | 271 | 272 | 129 | 145 | 273 | 60 | 274 | 275 | 276 | 277 | 278 | 279 | 280 | 281 | 69 | 1639 | 1640 |
| 636 | | | | | | | | | | | | | | | | | | | | |
| S24- | 282 | 283 | 284 | 285 | 286 | 190 | 287 | 288 | 60 | 289 | 290 | 291 | 30 | 292 | 32 | 33 | 293 | 85 | 1641 | 1642 |
| 740 | | | | | | | | | | | | | | | | | | | | |
| S24- | 294 | 295 | 296 | 297 | 298 | 24 | 7 | 299 | 44 | 300 | 301 | 302 | 135 | 303 | 304 | 182 | 305 | 53 | 1643 | 1644 |
| 791 | | | | | | | | | | | | | | | | | | | | |
| S24- | 306 | 307 | 308 | 157 | 309 | 310 | 160 | 161 | 147 | 311 | 312 | 313 | 314 | 315 | 316 | 317 | 318 | 69 | 1645 | 1646 |
| 902 | | | | | | | | | | | | | | | | | | | | |
| S24- | 319 | 320 | 321 | 322 | 323 | 324 | 7 | 325 | 44 | 326 | 327 | 328 | 249 | 329 | 330 | 252 | 253 | 53 | 1647 | 1648 |
| 921 | | | | | | | | | | | | | | | | | | | | |
| S24- | 331 | 332 | 56 | 333 | 334 | 24 | 7 | 335 | 60 | 336 | 337 | 338 | 135 | 303 | 50 | 182 | 339 | 53 | 1649 | 1650 |
| 1063 | | | | | | | | | | | | | | | | | | | | |
| S24- | 340 | 341 | 342 | 343 | 344 | 345 | 160 | 346 | 60 | 347 | 348 | 349 | 350 | 351 | 352 | 353 | 354 | 355 | 1651 | 1652 |
| 1224 | | | | | | | | | | | | | | | | | | | | |
| S24- | 356 | 357 | 358 | 359 | 360 | 361 | 130 | 362 | 60 | 363 | 364 | 365 | 366 | 367 | 368 | 369 | 370 | 69 | 1653 | 1654 |
| 1271 | | | | | | | | | | | | | | | | | | | | |
| S24- | 371 | 372 | 358 | 373 | 374 | 361 | 130 | 375 | 147 | 376 | 377 | 338 | 135 | 378 | 50 | 379 | 196 | 380 | 1655 | 1656 |
| 1339 | | | | | | | | | | | | | | | | | | | | |
| S24- | 381 | 382 | 242 | 243 | 383 | 245 | 7 | 115 | 60 | 384 | 385 | 386 | 387 | 388 | 389 | 252 | 253 | 85 | 1657 | 1658 |
| 1345 | | | | | | | | | | | | | | | | | | | | |
| S24- | 390 | 391 | 358 | 392 | 393 | 361 | 130 | 375 | 44 | 394 | 395 | 276 | 396 | 397 | 279 | 280 | 281 | 69 | 1659 | 1660 |
| 1378 | | | | | | | | | | | | | | | | | | | | |
| S24- | 398 | 399 | 56 | 4 | 400 | 24 | 7 | 115 | 147 | 401 | 402 | 403 | 404 | 405 | 121 | 122 | 406 | 69 | 1661 | 1662 |
| 1379 | | | | | | | | | | | | | | | | | | | | |
| S24- | 407 | 408 | 126 | 409 | 410 | 411 | 130 | 273 | 147 | 412 | 413 | 414 | 13 | 415 | 15 | 416 | 17 | 69 | 1663 | 1664 |
| 1384 | | | | | | | | | | | | | | | | | | | | |
| S24- | 417 | 418 | 199 | 419 | 420 | 202 | 203 | 259 | 421 | 422 | 423 | 207 | 208 | 424 | 210 | 182 | 211 | 53 | 1665 | 1666 |
| 1476 | | | | | | | | | | | | | | | | | | | | |
| S24- | 425 | 426 | 56 | 427 | 428 | 24 | 7 | 429 | 60 | 430 | 431 | 432 | 249 | 433 | 251 | 434 | 253 | 53 | 1667 | 1668 |
| 1564 | | | | | | | | | | | | | | | | | | | | |
| S24- | 435 | 436 | 437 | 142 | 438 | 144 | 145 | 146 | 60 | 439 | 440 | 178 | 441 | 442 | 181 | 182 | 183 | 443 | 1669 | 1670 |
| 1636 | | | | | | | | | | | | | | | | | | | | |
| S24- | 444 | 445 | 446 | 447 | 448 | 449 | 145 | 146 | 147 | 450 | 451 | 386 | 387 | 452 | 389 | 453 | 454 | 85 | 1671 | 1672 |
| 1002 | | | | | | | | | | | | | | | | | | | | |
| S24- | 455 | 456 | 457 | 458 | 459 | 460 | 42 | 461 | 60 | 462 | 463 | 464 | 465 | 466 | 467 | 468 | 469 | 69 | 1673 | 1674 |
| 1301 | | | | | | | | | | | | | | | | | | | | |
| S24- | 470 | 471 | 472 | 473 | 474 | 475 | 476 | 477 | 60 | 478 | 479 | 63 | 64 | 480 | 66 | 67 | 68 | 69 | 1675 | 1676 |
| 223 | | | | | | | | | | | | | | | | | | | | |
| S24- | 481 | 482 | 56 | 483 | 484 | 24 | 7 | 485 | 486 | 487 | 488 | 489 | 490 | 491 | 492 | 493 | 494 | 69 | 1677 | 1678 |
| 461 | | | | | | | | | | | | | | | | | | | | |
| S24- | 495 | 496 | 141 | 497 | 498 | 144 | 145 | 499 | 147 | 500 | 501 | 502 | 503 | 504 | 368 | 369 | 370 | 69 | 1679 | 1680 |
| 511 | | | | | | | | | | | | | | | | | | | | |
| S24- | 505 | 506 | 141 | 142 | 507 | 144 | 145 | 146 | 147 | 508 | 509 | 502 | 503 | 510 | 368 | 369 | 370 | 69 | 1681 | 1682 |
| 788 | | | | | | | | | | | | | | | | | | | | |
| S24- | 511 | 512 | 513 | 514 | 515 | 516 | 476 | 517 | 44 | 518 | 519 | 520 | 521 | 522 | 523 | 252 | 524 | 53 | 1683 | 1684 |
| 821 | | | | | | | | | | | | | | | | | | | | |
| S144- | 525 | 526 | 527 | 528 | 529 | 530 | 531 | 532 | 147 | 533 | 534 | 535 | 350 | 536 | 537 | 538 | 539 | 69 | 1685 | 1686 |
| 67 | | | | | | | | | | | | | | | | | | | | |
| S144- | 540 | 541 | 542 | 543 | 544 | 530 | 174 | 545 | 60 | 546 | 547 | 548 | 387 | 549 | 550 | 252 | 524 | 380 | 1687 | 1688 |
| 69 | | | | | | | | | | | | | | | | | | | | |
| S144- | 551 | 552 | 141 | 553 | 554 | 555 | 556 | 557 | 147 | 558 | 559 | 262 | 263 | 560 | 265 | 266 | 267 | 380 | 1689 | 1690 |
| 94 | | | | | | | | | | | | | | | | | | | | |
| S144- | 561 | 562 | 563 | 564 | 565 | 566 | 130 | 567 | 60 | 568 | 569 | 570 | 249 | 571 | 251 | 572 | 573 | 85 | 1691 | 1692 |
| 113 | | | | | | | | | | | | | | | | | | | | |
| S144- | 574 | 575 | 187 | 576 | 577 | 190 | 160 | 578 | 44 | 579 | 580 | 403 | 404 | 581 | 582 | 122 | 406 | 69 | 1693 | 1694 |
| 175 | | | | | | | | | | | | | | | | | | | | |
| S144- | 583 | 584 | 187 | 585 | 586 | 587 | 160 | 588 | 60 | 589 | 590 | 591 | 592 | 593 | 594 | 67 | 595 | 596 | 1695 | 1696 |
| 208 | | | | | | | | | | | | | | | | | | | | |
| S144- | 597 | 598 | 599 | 600 | 601 | 602 | 130 | 273 | 60 | 603 | 604 | 605 | 135 | 606 | 50 | 607 | 138 | 85 | 1697 | 1698 |
| 339 | | | | | | | | | | | | | | | | | | | | |
| S144- | 608 | 609 | 610 | 611 | 612 | 613 | 130 | 614 | 60 | 615 | 616 | 248 | 249 | 617 | 251 | 252 | 618 | 619 | 1699 | 1700 |
| 359 | | | | | | | | | | | | | | | | | | | | |
| S144- | 620 | 621 | 622 | 623 | 624 | 625 | 626 | 627 | 60 | 628 | 629 | 630 | 631 | 632 | 633 | 634 | 635 | 380 | 1701 | 1702 |
| 460 | | | | | | | | | | | | | | | | | | | | |
| S144- | 636 | 637 | 638 | 639 | 640 | 641 | 174 | 642 | 60 | 643 | 644 | 645 | 387 | 646 | 523 | 647 | 524 | 53 | 1703 | 1704 |
| 466 | | | | | | | | | | | | | | | | | | | | |
| S144- | 648 | 649 | 650 | 88 | 651 | 24 | 7 | 115 | 147 | 652 | 653 | 262 | 263 | 654 | 265 | 266 | 655 | 443 | 1705 | 1706 |
| 469 | | | | | | | | | | | | | | | | | | | | |
| S144- | 656 | 657 | 658 | 543 | 659 | 660 | 174 | 661 | 60 | 662 | 663 | 520 | 387 | 646 | 523 | 664 | 524 | 53 | 1707 | 1708 |
| 509 | | | | | | | | | | | | | | | | | | | | |
| S144- | 665 | 666 | 187 | 585 | 667 | 668 | 160 | 669 | 670 | 671 | 672 | 673 | 674 | 675 | 676 | 122 | 539 | 69 | 1709 | 1710 |
| 516 | | | | | | | | | | | | | | | | | | | | |
| S144- | 677 | 678 | 679 | 680 | 681 | 682 | 7 | 683 | 44 | 684 | 685 | 686 | 687 | 688 | 50 | 689 | 690 | 53 | 1711 | 1712 |
| 568 | | | | | | | | | | | | | | | | | | | | |
| S144- | 691 | 692 | 693 | 157 | 694 | 695 | 160 | 161 | 147 | 696 | 697 | 520 | 698 | 699 | 700 | 252 | 524 | 701 | 1713 | 1714 |
| 576 | | | | | | | | | | | | | | | | | | | | |
| S144- | 702 | 703 | 242 | 243 | 704 | 245 | 7 | 705 | 60 | 706 | 707 | 502 | 366 | 708 | 368 | 369 | 370 | 69 | 1715 | 1716 |
| 588 | | | | | | | | | | | | | | | | | | | | |
| S144- | 709 | 710 | 527 | 543 | 711 | 712 | 174 | 713 | 147 | 714 | 715 | 673 | 716 | 717 | 718 | 719 | 720 | 721 | 1717 | 1718 |
| 628 | | | | | | | | | | | | | | | | | | | | |
| S144- | 722 | 723 | 187 | 724 | 725 | 190 | 160 | 588 | 60 | 726 | 727 | 338 | 135 | 728 | 729 | 730 | 196 | 731 | 1719 | 1720 |
| 740 | | | | | | | | | | | | | | | | | | | | |
| S144- | 732 | 733 | 734 | 585 | 735 | 736 | 160 | 737 | 60 | 738 | 739 | 740 | 119 | 741 | 121 | 122 | 123 | 69 | 1721 | 1722 |
| 741 | | | | | | | | | | | | | | | | | | | | |
| S144- | 742 | 743 | 744 | 543 | 745 | 746 | 174 | 747 | 60 | 748 | 749 | 520 | 387 | 750 | 523 | 252 | 524 | 751 | 1723 | 1724 |
| 803 | | | | | | | | | | | | | | | | | | | | |
| S144- | 752 | 753 | 754 | 755 | 756 | 757 | 145 | 758 | 60 | 759 | 760 | 761 | 208 | 762 | 50 | 182 | 196 | 380 | 1725 | 1726 |
| 843 | | | | | | | | | | | | | | | | | | | | |
| S144- | 763 | 764 | 765 | 497 | 766 | 144 | 145 | 499 | 60 | 767 | 768 | 769 | 770 | 771 | 251 | 252 | 772 | 85 | 1727 | 1728 |
| 877 | | | | | | | | | | | | | | | | | | | | |
| S144- | 773 | 774 | 775 | 776 | 777 | 778 | 160 | 779 | 60 | 780 | 781 | 782 | 30 | 783 | 32 | 33 | 293 | 53 | 1729 | 1730 |
| 952 | | | | | | | | | | | | | | | | | | | | |
| S144- | 784 | 785 | 786 | 787 | 788 | 789 | 790 | 791 | 60 | 792 | 793 | 794 | 30 | 795 | 32 | 33 | 293 | 53 | 1731 | 1732 |
| 971 | | | | | | | | | | | | | | | | | | | | |
| S144- | 796 | 797 | 798 | 799 | 800 | 801 | 7 | 115 | 60 | 802 | 803 | 804 | 30 | 805 | 32 | 806 | 293 | 53 | 1733 | 1734 |
| 1036 | | | | | | | | | | | | | | | | | | | | |
| S144- | 807 | 808 | 693 | 809 | 810 | 811 | 160 | 812 | 813 | 814 | 815 | 816 | 135 | 817 | 50 | 182 | 818 | 380 | 1735 | 1736 |
| 1079 | | | | | | | | | | | | | | | | | | | | |
| S144- | 819 | 820 | 56 | 821 | 822 | 24 | 7 | 823 | 60 | 824 | 825 | 403 | 404 | 826 | 121 | 122 | 406 | 69 | 1737 | 1738 |
| 1299 | | | | | | | | | | | | | | | | | | | | |
| S144- | 827 | 828 | 829 | 830 | 831 | 832 | 160 | 833 | 834 | 835 | 836 | 837 | 64 | 838 | 66 | 839 | 68 | 69 | 1739 | 1740 |
| 1339 | | | | | | | | | | | | | | | | | | | | |
| S144- | 840 | 841 | 842 | 843 | 844 | 190 | 287 | 845 | 670 | 846 | 847 | 520 | 387 | 646 | 523 | 252 | 524 | 53 | 1741 | 1742 |
| 1406 | | | | | | | | | | | | | | | | | | | | |
| S144- | 848 | 849 | 850 | 851 | 852 | 310 | 160 | 853 | 44 | 854 | 855 | 520 | 387 | 856 | 523 | 252 | 857 | 380 | 1743 | 1744 |
| 1407 | | | | | | | | | | | | | | | | | | | | |
| S144- | 858 | 859 | 860 | 861 | 862 | 863 | 160 | 864 | 147 | 865 | 866 | 867 | 868 | 869 | 870 | 871 | 872 | 69 | 1745 | 1746 |
| 1569 | | | | | | | | | | | | | | | | | | | | |
| S144- | 873 | 874 | 542 | 875 | 876 | 877 | 174 | 878 | 60 | 879 | 880 | 881 | 387 | 882 | 883 | 252 | 884 | 885 | 1747 | 1748 |
| 1641 | | | | | | | | | | | | | | | | | | | | |
| S144- | 886 | 887 | 888 | 889 | 890 | 891 | 145 | 892 | 60 | 893 | 894 | 895 | 208 | 303 | 50 | 182 | 196 | 896 | 1749 | 1750 |
| 1827 | | | | | | | | | | | | | | | | | | | | |
| S144- | 897 | 898 | 126 | 899 | 900 | 602 | 130 | 901 | 44 | 902 | 903 | 904 | 905 | 906 | 121 | 122 | 406 | 907 | 1751 | 1752 |
| 1848 | | | | | | | | | | | | | | | | | | | | |
| S144- | 908 | 909 | 610 | 910 | 911 | 613 | 130 | 912 | 60 | 913 | 914 | 645 | 915 | 916 | 523 | 252 | 524 | 53 | 1753 | 1754 |
| 1850 | | | | | | | | | | | | | | | | | | | | |
| S144- | 917 | 918 | 919 | 920 | 921 | 310 | 160 | 161 | 60 | 922 | 923 | 291 | 30 | 924 | 925 | 33 | 926 | 53 | 1755 | 1756 |
| 2234 | | | | | | | | | | | | | | | | | | | | |
| S564- | 927 | 928 | 929 | 930 | 931 | 932 | 25 | 115 | 220 | 933 | 934 | 935 | 151 | 936 | 66 | 67 | 68 | 937 | 1757 | 1758 |
| 105 | | | | | | | | | | | | | | | | | | | | |
| S564- | 938 | 939 | 270 | 940 | 941 | 942 | 943 | 944 | 60 | 945 | 946 | 12 | 947 | 948 | 949 | 950 | 17 | 18 | 1759 | 1760 |
| 14 | | | | | | | | | | | | | | | | | | | | |
| S564- | 951 | 952 | 187 | 953 | 954 | 955 | 160 | 956 | 60 | 957 | 958 | 63 | 94 | 959 | 96 | 67 | 960 | 69 | 1761 | 1762 |
| 68 | | | | | | | | | | | | | | | | | | | | |
| S564- | 961 | 962 | 56 | 4 | 963 | 24 | 7 | 115 | 60 | 964 | 965 | 432 | 249 | 966 | 251 | 252 | 967 | 53 | 1763 | 1764 |
| 98 | | | | | | | | | | | | | | | | | | | | |
| S564- | 927 | 928 | 929 | 930 | 931 | 932 | 25 | 115 | 220 | 933 | 934 | 935 | 151 | 936 | 66 | 67 | 68 | 937 | 1757 | 1758 |
| 105 | | | | | | | | | | | | | | | | | | | | |
| S564- | 968 | 969 | 187 | 953 | 970 | 190 | 160 | 971 | 60 | 972 | 973 | 63 | 974 | 975 | 96 | 67 | 976 | 69 | 1765 | 1766 |
| 134 | | | | | | | | | | | | | | | | | | | | |
| S564- | 977 | 978 | 979 | 980 | 981 | 982 | 160 | 983 | 44 | 984 | 985 | 63 | 151 | 986 | 66 | 67 | 987 | 18 | 1767 | 1768 |
| 138 | | | | | | | | | | | | | | | | | | | | |
| S564- | 988 | 989 | 990 | 991 | 992 | 144 | 145 | 499 | 60 | 993 | 994 | 995 | 770 | 996 | 251 | 252 | 997 | 380 | 1769 | 1770 |
| 152 | | | | | | | | | | | | | | | | | | | | |
| S564- | 998 | 999 | 308 | 1000 | 1001 | 310 | 160 | 1002 | 44 | 1003 | 1004 | 63 | 94 | 1005 | 96 | 67 | 1006 | 69 | 1771 | 1772 |
| 218 | | | | | | | | | | | | | | | | | | | | |
| S564- | 1007 | 1008 | 1009 | 1010 | 1011 | 1012 | 1013 | 1014 | 60 | 1015 | 1016 | 1017 | 64 | 1018 | 66 | 1019 | 1020 | 69 | 1773 | 1774 |
| 249 | | | | | | | | | | | | | | | | | | | | |
| S564- | 1021 | 1022 | 187 | 1023 | 1024 | 190 | 160 | 1025 | 60 | 1026 | 1027 | 1028 | 94 | 1029 | 96 | 67 | 1006 | 721 | 1775 | 1776 |
| 265 | | | | | | | | | | | | | | | | | | | | |
| S564- | 1030 | 1031 | 56 | 333 | 1032 | 24 | 7 | 1033 | 60 | 1034 | 1035 | 1036 | 249 | 1037 | 1038 | 252 | 1039 | 85 | 1777 | 1778 |
| 275 | | | | | | | | | | | | | | | | | | | | |
| S564- | 1040 | 1041 | 187 | 953 | 1042 | 190 | 160 | 1043 | 60 | 1044 | 1045 | 63 | 64 | 1046 | 66 | 67 | 68 | 69 | 1779 | 1780 |
| 287 | | | | | | | | | | | | | | | | | | | | |
| S166- | 1047 | 1048 | 1049 | 1050 | 1051 | 1052 | 1053 | 1054 | 1055 | 1056 | 1057 | 1058 | 387 | 1059 | 523 | 252 | 524 | 53 | 1781 | 1782 |
| 32 | | | | | | | | | | | | | | | | | | | | |
| S305- | 1060 | 1061 | 1062 | 1063 | 1064 | 1065 | 145 | 1066 | 1067 | 1068 | 1069 | 1070 | 441 | 1071 | 181 | 1072 | 183 | 1073 | 1783 | 1784 |
| 223 | | | | | | | | | | | | | | | | | | | | |
| S305- | 1074 | 1075 | 457 | 458 | 1076 | 1077 | 42 | 1078 | 1079 | 1080 | 1081 | 1082 | 208 | 1083 | 210 | 182 | 1084 | 53 | 1785 | 1786 |
| 399 | | | | | | | | | | | | | | | | | | | | |
| S305- | 1085 | 1086 | 457 | 1087 | 1088 | 1077 | 42 | 1078 | 60 | 1089 | 1090 | 1091 | 208 | 1092 | 210 | 182 | 211 | 443 | 1787 | 1788 |
| 1456 | | | | | | | | | | | | | | | | | | | | |
| R125 | 1093 | 1094 | 1062 | 1095 | 1096 | 1097 | 145 | 1098 | 147 | 1099 | 1100 | 1101 | 1102 | 1103 | 1104 | 1105 | 1106 | 18 | 1789 | 1790 |
| -306 | | | | | | | | | | | | | | | | | | | | |
| R125 | 1107 | 1108 | 1109 | 4 | 1110 | 1111 | 7 | 1112 | 147 | 1113 | 1114 | 1115 | 1116 | 1117 | 1118 | 1119 | 1120 | 69 | 1791 | 1792 |
| -444 | | | | | | | | | | | | | | | | | | | | |
| R3- | 1121 | 1122 | 1123 | 1124 | 1125 | 1126 | 476 | 1127 | 60 | 1128 | 1129 | 1130 | 1131 | 1132 | 1133 | 1134 | 253 | 53 | 1793 | 1794 |
| 428 | | | | | | | | | | | | | | | | | | | | |
| R478 | 1135 | 1136 | 610 | 1137 | 1138 | 1139 | 130 | 499 | 60 | 1140 | 1141 | 1142 | 1143 | 1144 | 1145 | 1146 | 196 | 701 | 1795 | 1796 |
| 910- | | | | | | | | | | | | | | | | | | | | |
| 171 | | | | | | | | | | | | | | | | | | | | |
| R478 | 1147 | 1148 | 141 | 142 | 1149 | 144 | 145 | 146 | 60 | 1150 | 1151 | 1152 | 249 | 1153 | 1154 | 252 | 253 | 380 | 1797 | 1798 |
| 910- | | | | | | | | | | | | | | | | | | | | |
| 23 | | | | | | | | | | | | | | | | | | | | |
| R478 | 1155 | 1156 | 1157 | 1158 | 1159 | 1160 | 1161 | 1162 | 60 | 1163 | 1164 | 1165 | 1166 | 1167 | 251 | 453 | 1168 | 443 | 1799 | 1800 |
| 910- | | | | | | | | | | | | | | | | | | | | |
| 25 | | | | | | | | | | | | | | | | | | | | |
| R478 | 1169 | 1170 | 1171 | 1172 | 1173 | 1174 | 130 | 1175 | 1176 | 1177 | 1178 | 248 | 249 | 1179 | 1180 | 1181 | 253 | 85 | 1801 | 1802 |
| 910-3 | | | | | | | | | | | | | | | | | | | | |
| R478 | 1182 | 1183 | 1184 | 1185 | 1186 | 1187 | 145 | 273 | 1188 | 1189 | 1190 | 1191 | 1192 | 1193 | 1194 | 1195 | 1196 | 69 | 1803 | 1804 |
| 910- | | | | | | | | | | | | | | | | | | | | |
| 421 | | | | | | | | | | | | | | | | | | | | |
| R478 | 1197 | 1198 | 126 | 127 | 1199 | 613 | 130 | 273 | 9 | 1200 | 1201 | 338 | 135 | 1202 | 50 | 182 | 196 | 380 | 1805 | 1806 |
| 910-8 | | | | | | | | | | | | | | | | | | | | |
| S195- | 1203 | 1204 | 1205 | 1206 | 1207 | 144 | 145 | 1208 | 147 | 1209 | 1210 | 338 | 1211 | 1212 | 50 | 182 | 196 | 1213 | 1807 | 1808 |
| 637 | | | | | | | | | | | | | | | | | | | | |
| S380- | 1214 | 1215 | 1216 | 1217 | 1218 | 613 | 130 | 1219 | 60 | 1220 | 1221 | 673 | 350 | 1222 | 537 | 122 | 539 | 1223 | 1809 | 1810 |
| 1191 | | | | | | | | | | | | | | | | | | | | |
| S451- | 1224 | 1225 | 1226 | 1227 | 1228 | 310 | 1229 | 161 | 60 | 1230 | 1231 | 673 | 1232 | 1222 | 537 | 719 | 539 | 18 | 1811 | 1812 |
| 101 | | | | | | | | | | | | | | | | | | | | |
| S451- | 1233 | 1234 | 1235 | 1236 | 1237 | 1238 | 1239 | 1240 | 60 | 1241 | 1242 | 1243 | 592 | 1244 | 594 | 1245 | 1246 | 69 | 1813 | 1814 |
| 11 | | | | | | | | | | | | | | | | | | | | |
| S451- | 1247 | 1248 | 1249 | 1250 | 1251 | 324 | 25 | 1252 | 60 | 1253 | 1254 | 570 | 249 | 1255 | 251 | 252 | 253 | 53 | 1815 | 1816 |
| 1101 | | | | | | | | | | | | | | | | | | | | |
| S451- | 1256 | 1257 | 1258 | 1259 | 1260 | 1261 | 130 | 1262 | 60 | 1263 | 1264 | 769 | 1265 | 1266 | 251 | 1267 | 1268 | 1269 | 1817 | 1818 |
| 1439 | | | | | | | | | | | | | | | | | | | | |
| S451- | 1203 | 1204 | 1205 | 1206 | 1207 | 144 | 145 | 1208 | 147 | 1209 | 1210 | 338 | 1211 | 1212 | 50 | 182 | 196 | 1213 | 1807 | 1808 |
| 1451 | | | | | | | | | | | | | | | | | | | | |
| S451- | 1270 | 1271 | 1272 | 1273 | 1274 | 1275 | 1276 | 1277 | 421 | 1278 | 1279 | 63 | 1280 | 1281 | 594 | 1282 | 1283 | 69 | 1819 | 1820 |
| 1477 | | | | | | | | | | | | | | | | | | | | |
| S451- | 1182 | 1183 | 1184 | 1185 | 1186 | 1187 | 145 | 273 | 1188 | 1189 | 1190 | 1191 | 1192 | 1193 | 1194 | 1195 | 1196 | 69 | 1803 | 1804 |
| 1503 | | | | | | | | | | | | | | | | | | | | |
| S451- | 1214 | 1215 | 1216 | 1217 | 1218 | 613 | 130 | 1219 | 60 | 1220 | 1221 | 673 | 350 | 1222 | 537 | 122 | 539 | 1223 | 1809 | 1810 |
| 1522 | | | | | | | | | | | | | | | | | | | | |
| S451- | 1284 | 1285 | 72 | 1286 | 1287 | 1288 | 1289 | 1290 | 60 | 1291 | 1292 | 1293 | 30 | 1294 | 32 | 1295 | 1296 | 53 | 1821 | 1822 |
| 1921 | | | | | | | | | | | | | | | | | | | | |
| S451- | 1297 | 1298 | 1299 | 1300 | 1301 | 1302 | 476 | 1303 | 60 | 1304 | 1305 | 63 | 151 | 1306 | 1307 | 1308 | 68 | 69 | 1823 | 1824 |
| 337 | | | | | | | | | | | | | | | | | | | | |
| S451- | 1309 | 1310 | 1311 | 4 | 1312 | 1313 | 7 | 1314 | 1315 | 1316 | 1317 | 816 | 135 | 1318 | 50 | 182 | 196 | 701 | 1825 | 1826 |
| 650 | | | | | | | | | | | | | | | | | | | | |
| S626- | 1224 | 1225 | 1226 | 1227 | 1228 | 310 | 1229 | 161 | 60 | 1230 | 1231 | 673 | 1232 | 1222 | 537 | 719 | 539 | 18 | 1811 | 1812 |
| 362 | | | | | | | | | | | | | | | | | | | | |
| S626- | 1256 | 1257 | 1258 | 1259 | 1260 | 1261 | 130 | 1262 | 60 | 1263 | 1264 | 769 | 1265 | 1266 | 251 | 1267 | 1268 | 1269 | 1817 | 1818 |
| 651 | | | | | | | | | | | | | | | | | | | | |
| S626- | 1319 | 1320 | 1321 | 1322 | 1323 | 1324 | 1325 | 1326 | 670 | 1327 | 1328 | 1329 | 1330 | 1331 | 1194 | 1332 | 1196 | 69 | 1827 | 1828 |
| 692 | | | | | | | | | | | | | | | | | | | | |
| S626- | 1319 | 1320 | 1321 | 1322 | 1323 | 1324 | 1325 | 1326 | 670 | 1327 | 1328 | 1329 | 1330 | 1331 | 1194 | 1332 | 1196 | 69 | 1827 | 1828 |
| 7 | | | | | | | | | | | | | | | | | | | | |
| S626- | 1270 | 1271 | 1272 | 1273 | 1274 | 1275 | 1276 | 1277 | 421 | 1278 | 1279 | 63 | 1280 | 1281 | 594 | 1282 | 1283 | 69 | 1819 | 1820 |
| 747 | | | | | | | | | | | | | | | | | | | | |
| S626- | 1284 | 1285 | 72 | 1286 | 1287 | 1288 | 1289 | 1290 | 60 | 1291 | 1292 | 1293 | 30 | 1294 | 32 | 1295 | 1296 | 53 | 1821 | 1822 |
| 75 | | | | | | | | | | | | | | | | | | | | |
| S626- | 1333 | 1334 | 1335 | 1336 | 1337 | 1338 | 145 | 1339 | 9 | 1340 | 1341 | 1342 | 1343 | 1344 | 1345 | 1346 | 1347 | 53 | 1829 | 1830 |
| 8 | | | | | | | | | | | | | | | | | | | | |
| S68- | 1348 | 1349 | 1350 | 1351 | 1352 | 1353 | 1354 | 1355 | 1356 | 1357 | 1358 | 1359 | 30 | 1360 | 925 | 33 | 293 | 380 | 1831 | 1832 |
| 253 | | | | | | | | | | | | | | | | | | | | |
| S728- | 1361 | 1362 | 187 | 585 | 1363 | 190 | 160 | 588 | 60 | 1364 | 1365 | 63 | 151 | 1366 | 66 | 67 | 68 | 69 | 1833 | 1834 |
| 1502 | | | | | | | | | | | | | | | | | | | | |
| S728- | 1367 | 1368 | 1369 | 1370 | 1371 | 613 | 1372 | 1373 | 60 | 1374 | 1201 | 338 | 135 | 1375 | 50 | 182 | 196 | 85 | 1835 | 1836 |
| 1789 | | | | | | | | | | | | | | | | | | | | |
| S728- | 1361 | 1362 | 187 | 585 | 1363 | 190 | 160 | 588 | 60 | 1364 | 1365 | 63 | 151 | 1366 | 66 | 67 | 68 | 69 | 1833 | 1834 |
| 1806 | | | | | | | | | | | | | | | | | | | | |
| S728- | 1376 | 1377 | 1378 | 1379 | 1380 | 1381 | 160 | 1382 | 1315 | 1383 | 1384 | 1385 | 441 | 1386 | 181 | 182 | 183 | 85 | 1837 | 1838 |
| 1981 | | | | | | | | | | | | | | | | | | | | |
| S728- | 1387 | 1388 | 1389 | 953 | 1390 | 1391 | 160 | 1392 | 60 | 1393 | 1394 | 1395 | 1396 | 1397 | 66 | 1398 | 1399 | 69 | 1839 | 1840 |
| 2036 | | | | | | | | | | | | | | | | | | | | |
| S728- | 1367 | 1368 | 1369 | 1370 | 1371 | 613 | 1372 | 1373 | 60 | 1374 | 1201 | 338 | 135 | 1375 | 50 | 182 | 196 | 85 | 1835 | 1836 |
| 2111 | | | | | | | | | | | | | | | | | | | | |
| S728- | 1147 | 1148 | 141 | 142 | 1149 | 144 | 145 | 146 | 60 | 1150 | 1151 | 1152 | 249 | 1153 | 1154 | 252 | 253 | 380 | 1797 | 1798 |
| 2148 | | | | | | | | | | | | | | | | | | | | |
| S728- | 1400 | 1401 | 1402 | 1403 | 1404 | 566 | 130 | 1405 | 60 | 1406 | 1407 | 1036 | 249 | 1408 | 251 | 252 | 1409 | 443 | 1841 | 1842 |
| 656 | | | | | | | | | | | | | | | | | | | | |
| S728- | 1376 | 1377 | 1378 | 1379 | 1380 | 1381 | 160 | 1382 | 1315 | 1383 | 1384 | 1385 | 441 | 1386 | 181 | 182 | 183 | 85 | 1837 | 1838 |
| 723 | | | | | | | | | | | | | | | | | | | | |
| S728- | 1182 | 1183 | 1184 | 1185 | 1186 | 1187 | 145 | 273 | 1188 | 1189 | 1190 | 1191 | 1192 | 1193 | 1194 | 1195 | 1196 | 69 | 1803 | 1804 |
| 826 | | | | | | | | | | | | | | | | | | | | |
| S728- | 1387 | 1388 | 1389 | 953 | 1390 | 1391 | 160 | 1392 | 60 | 1393 | 1394 | 1395 | 1396 | 1397 | 66 | 1398 | 1399 | 69 | 1839 | 1840 |
| 959 | | | | | | | | | | | | | | | | | | | | |
| S210- | 1410 | 1411 | 1412 | 1413 | 1414 | 190 | 1415 | 1416 | 60 | 1417 | 1418 | 1395 | 94 | 1419 | 66 | 67 | 68 | 18 | 1843 | 1844 |
| 530 | | | | | | | | | | | | | | | | | | | | |
| S210- | 1420 | 1421 | 199 | 1422 | 1423 | 1424 | 104 | 1425 | 60 | 1426 | 1427 | 1428 | 1429 | 1430 | 1431 | 1432 | 1433 | 69 | 1845 | 1846 |
| 1129 | | | | | | | | | | | | | | | | | | | | |
| S451- | 1434 | 1435 | 798 | 1436 | 1437 | 1438 | 7 | 1439 | 44 | 1440 | 1441 | 1442 | 208 | 1443 | 210 | 182 | 1444 | 380 | 1847 | 1848 |
| 5 | | | | | | | | | | | | | | | | | | | | |
| S451- | 1445 | 1446 | 1447 | 473 | 1448 | 1449 | 1450 | 1451 | 60 | 1452 | 1453 | 63 | 151 | 1454 | 66 | 67 | 1246 | 69 | 1849 | 1850 |
| 506 | | | | | | | | | | | | | | | | | | | | |
| S451- | 1455 | 1456 | 1457 | 1458 | 1459 | 1460 | 1461 | 146 | 60 | 1462 | 1463 | 1464 | 1465 | 1466 | 1467 | 1468 | 1469 | 53 | 1851 | 1852 |
| 1140 | | | | | | | | | | | | | | | | | | | | |
| S451- | 1470 | 1471 | 1472 | 1473 | 1474 | 1475 | 476 | 517 | 60 | 1476 | 1477 | 1478 | 119 | 1479 | 121 | 122 | 123 | 69 | 1853 | 1854 |
| 1190 | | | | | | | | | | | | | | | | | | | | |
| S626- | 1480 | 1481 | 1482 | 1483 | 1484 | 1485 | 7 | 115 | 44 | 1486 | 1487 | 338 | 1143 | 1488 | 50 | 1489 | 196 | 53 | 1855 | 1856 |
| 84 | | | | | | | | | | | | | | | | | | | | |
| S626- | 1490 | 1491 | 1492 | 243 | 1493 | 245 | 7 | 1494 | 44 | 1495 | 1496 | 1497 | 1498 | 1499 | 1500 | 1501 | 1502 | 18 | 1857 | 1858 |
| 161 | | | | | | | | | | | | | | | | | | | | |
| S626- | 1503 | 1504 | 1505 | 953 | 1506 | 1507 | 160 | 1508 | 60 | 1509 | 1510 | 63 | 592 | 1511 | 66 | 1512 | 68 | 69 | 1859 | 1860 |
| 664 | | | | | | | | | | | | | | | | | | | | |
| S728- | 1513 | 1514 | 1515 | 1516 | 1517 | 1518 | 476 | 1519 | 670 | 1520 | 1521 | 1522 | 1523 | 1524 | 1525 | 1526 | 253 | 85 | 1861 | 1862 |
| 209 | | | | | | | | | | | | | | | | | | | | |
| S728- | 1527 | 1528 | 1235 | 1529 | 1530 | 1531 | 1239 | 1532 | 147 | 1533 | 1534 | 1535 | 1536 | 1537 | 1538 | 1181 | 1539 | 1540 | 1863 | 1864 |
| 369 | | | | | | | | | | | | | | | | | | | | |
| S728- | 1541 | 1542 | 358 | 392 | 1543 | 1544 | 130 | 146 | 44 | 1545 | 1546 | 1547 | 770 | 1548 | 251 | 252 | 997 | 85 | 1865 | 1866 |
| 430 | | | | | | | | | | | | | | | | | | | | |
| S728- | 1549 | 1550 | 1551 | 1552 | 1553 | 1381 | 1554 | 1555 | 44 | 1556 | 1557 | 1558 | 249 | 1559 | 1560 | 1561 | 1562 | 85 | 1867 | 1868 |
| 537 | | | | | | | | | | | | | | | | | | | | |
| S728- | 1563 | 1564 | 1565 | 1566 | 1567 | 1568 | 130 | 1569 | 60 | 1570 | 1571 | 1572 | 1573 | 1574 | 1575 | 950 | 1576 | 69 | 1869 | 1870 |
| 1157 | | | | | | | | | | | | | | | | | | | | |
| S728- | 1577 | 1578 | 1579 | 1580 | 1581 | 217 | 218 | 1582 | 670 | 1583 | 1584 | 1585 | 135 | 1586 | 1587 | 182 | 196 | 443 | 1871 | 1872 |
| 1261 | | | | | | | | | | | | | | | | | | | | |
| S728- | 1588 | 1589 | 1590 | 1591 | 1592 | 1593 | 1594 | 1595 | 1596 | 1597 | 1598 | 1599 | 135 | 1600 | 1601 | 1489 | 1602 | 53 | 1873 | 1874 |
| 1690 | | | | | | | | | | | | | | | | | | | | |

### 1. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### 2. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. Areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, the substitution of amino acids whose hydropathy indices are within ±2 is included. Those that are within ±1 may be included, or those within ±0.5 may be included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. The greatest local average hydrophilicity of a protein, as governed by the hydrophilicity of its adjacent amino acids, may correlate with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, the substitution of amino acids whose hydrophilicity values are within +2 may be included, or those which are within +1 may be included, or those within +0.5 may be included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

The amino acid substitutions may be made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (e.g. conservative amino acid substitutions) may be made in the naturally occurring sequence. Substitutions can be made in that portion of the antibody that lies outside the domain(s) forming intermolecular contacts. Conservative amino acid substitutions can be used that do not substantially change the structural characteristics of the protein or polypeptide (e.g., one or more replacement amino acids that do not disrupt the secondary structure that characterizes the native antibody).

### VII. Nucleic Acids

Nucleic acid sequences can exist in a variety of instances such as: isolated segments and recombinant vectors of incorporated sequences or recombinant polynucleotides encoding peptides and polypeptides of the disclosure, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

Also included are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). The isolated polynucleotide may comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N. Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antigenic peptide or polypeptide) that it encodes. Mutations can be introduced using any technique known in the art. One or more particular amino acid residues may be changed using, for example, a site-directed mutagenesis protocol. One or more randomly selected residues may be changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

Nucleic acid molecules may be suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

The nucleic acid molecules may be used as probes or PCR primers for specific nucleic acid sequences. For instance, a nucleic acid molecule probe may be used in diagnostic methods or a nucleic acid molecule PCR primer may be used to amplify regions of DNA that could be used, inter alia, to isolate nucleic acid sequences for use in producing the engineered cells of the disclosure. The nucleic acid molecules may be further defined as oligonucleotides.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### VIII. Polypeptide Expression

Also provided are nucleic acid molecule encoding polypeptides, antibodies, or antigen binding fragments of the disclosure. The nucleic acid molecules may be used to express large quantities of polypeptides. If the nucleic acid molecules are derived from a non-human, non-transgenic animal, the nucleic acid molecules may be used for humanization of the antibody or TCR genes.

### A. Vectors

Contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. Also provided are expression vectors comprising nucleic acid molecules may encode fusion proteins, modified antibodies, antibody heavy and/or light chain, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. A vector that encodes a functionally complete human CH or CL immunoglobulin sequence with appropriate restriction sites may be engineered so that any VH or VL sequence can be easily inserted and expressed. A vector that encodes a functionally complete human TCR alpha or TCR beta sequence with appropriate restriction sites may be engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### B. Expression Systems

Numerous expression systems exist that comprise at least a part or all of the expression vectors discussed above. Prokaryote- and/or eukaryote-based systems can be employed to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### C. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### IX. Pharmaceutical Compositions

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection. Compositions of the present disclosure (e.g., compositions comprising SARS-CoV-2 protein-binding polypeptides) may be administered to a subject intravenously.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In instances where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). One or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. A unit dose may comprise a single administrable dose.

The quantity to be administered, both according to number of treatments and unit dose, depends on the treatment effect desired. An effective dose is understood to refer to an amount necessary to achieve a particular effect. It is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

The therapeutically effective or sufficient amount of the immune checkpoint inhibitor, such as an antibody and/or microbial modulator, that is administered to a human may be in the range of about 0.01 to about 50 mg/kg of patient body weight whether by one or more administrations. The therapy used may be about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. A therapy described herein may be administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

The effective dose of the pharmaceutical composition may be one which can provide a blood level of about 1 μM to 150 μM. The effective dose may provide a blood level of about 4 μM to 100 μM; or about 1μ M to 100μ; or about 1μ M to 50μ; or about 1μ M to 40μ; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). The dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. The therapeutic agent may be administered to a subject and may be metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### X. Detectable Labels

It will be useful to detectably or therapeutically label an antibody or antigen binding fragment. Methods for conjugating polypeptides to these agents are known in the art. For the purpose of illustration only, polypeptides can be labeled with a detectable moiety such as a radioactive atom, a chromophore, a fluorophore, or the like. Such labeled polypeptides can be used for diagnostic techniques, either in vivo, or in an isolated test sample or in methods described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.).

The fluorescent label may be functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/polypeptides, e.g., rhodamine/anti-rhodamine, biotin/avidin and biotin/strepavidin.

The coupling of polypeptides to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten polypeptides. See, Harlow and Lane (1988) supra.

### XI. Sample Preparation

Methods can involve obtaining or evaluating a sample from a subject. The sample may include a sample obtained from any source including but not limited to blood, sweat, hair follicle, buccal tissue, tears, menses, feces, or saliva. Any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. The samples may be obtained by biopsy. The sample may be obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

The biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. A molecular profiling business may consult on which assays or tests are most appropriately indicated. The patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. Multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In some cases, the fine needle aspirate sampling procedure may be guided by the use of an ultrasound, X-ray, or other imaging device.

The molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

A medical professional may need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

The subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### XII. Host Cells

As used herein, the terms “cell,” “cell line,” and “cell culture” may be used interchangeably. All of these terms also include both freshly isolated cells and ex vivo cultured, activated or expanded cells. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Transfection can be carried out on any prokaryotic or eukaryotic cell. Electroporation can involve transfection of a human cell. Electroporation can involve transfection of an animal cell. Transfection can involve transfection of a cell line or a hybrid cell type. The cells or cell lines can be A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293 FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NS0, Panc-1, PC12, PC-3, Peripheral blood cells, Plasma cells, Primary Fibroblasts, RBL, Renca, RLE, SF21, SF9, SH-SY5Y, SK-MES-1, SK-N-SH, SL3, SW403, Stimulus-triggered Acquisition of Pluripotency (STAP) cell or derivate SW403, T-cells, THP-1, Tumor cells, U2OS, U937, peripheral blood lymphocytes, expanded T cells, hematopoietic stem cells, or Vero cells.

### XIII. Kits

Also described are kits containing compositions of the disclosure or compositions to implement methods of the disclosure. Kits can be used to detect the presence of a SARS-CoV-2 virus in a sample. A kit can contain, contain at least or contain at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. A kit can contain one or more polypeptides capable of binding to a SARS-CoV-2 spike protein, including polypeptides disclosed herein. For example, a kit may comprise at least 1, 2, 3, 4, 5, 6, 7, 8, 9, 10 or more Fabs disclosed herein for detecting a SARS-CoV-2 spike protein. A kit may comprise a detection pair. A kit may comprise an enzyme. A kit may comprise a substrate for an enzyme.

Kits may comprise components, which may be individually packaged or placed in a container, such as a tube, bottle, vial, syringe, or other suitable container means.

Individual components may also be provided in a kit in concentrated amounts; a component may be provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Negative and/or positive control nucleic acids, probes, and inhibitors may be included in some kits.

Kits may further comprise instructions for use. For example, a kit comprises instructions for detecting a SARS-CoV-2 virus in a sample.

It is contemplated that any method or composition described herein can be implemented with respect to any other method or composition described herein and that different aspects may be combined. The claims originally filed are contemplated to cover claims that are multiply dependent on any filed claim or combination of filed claims.

### XIV. EXAMPLES

The following examples are included to demonstrate certain aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments and aspects which are disclosed and still obtain a like or similar result without departing from the spirit and scope of the invention.

### Example 1-Cross Neutralization of Emerging SARS-CoV-2 Variants of Concern by Antibodies Targeting Distinct Epitopes on Spike

### A. Results

1. Convalescent Sera have Reduced Antibody Titers but Retain Neutralization Capabilities against Circulating SARS-CoV-2 VOCs

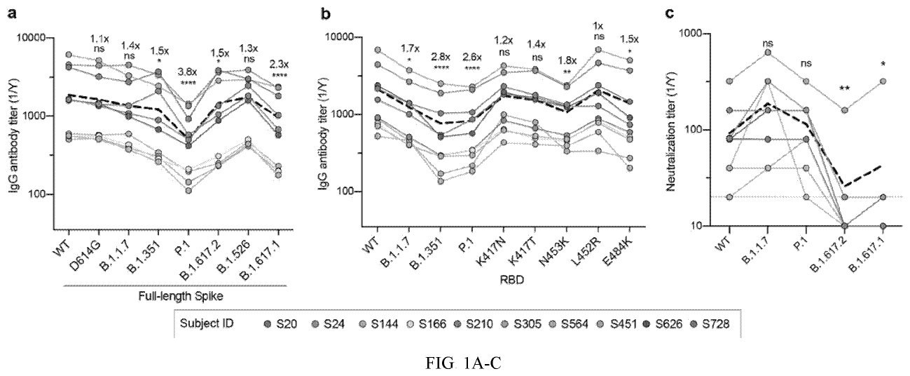

To investigate whether antibodies from subjects naturally infected with WT SARS-CoV-2 lost binding or neutralization activity against VOCs, the inventors collected blood samples from 10 convalescent donors at a median of 49 days post-symptom onset^{26, 27} (Supplementary Table 1) forin-depth analysis of specificity of individual memory B cells. As an initial estimate of antibody activity from these patients, serum antibody reactivity was measured comparing reactivity to WTtrimeric SARS-CoV-2 spike and spike proteins from the D614G, B.1.1.7, B. 1.351, P.1, B.1.617.2, B.1.526, and B.1.617.1 variants. While serum antibody IgG titers from these 10 patients against WT and D614G spike antigens were similar, titers were significantly reduced against the spike proteins of B.1.1.7 (1.4-fold), B.1.351 (1.5-fold), P.1 (3.8-fold), B.1.617.2 (1.5-fold), B.1.526 (1.3-fold), and B.1.617.1 (2.3-fold) relative to WT spike protein (FIG. 1a). Similarly, IgG titers against the RBD of B.1.1.7 (1.7-fold), B.1.351 (2.8-fold) and P.1 (2.6-fold) were reduced compared to WT RBD. However, the inventors noted that there was less than a 2-fold decrease in antibody binding against single mutants of the RBD (FIG. 1b). Despite reductions in serum binding activity, the sera retained similar neutralizing titers against the WT, B.1.1.7 and P.1 SARS-CoV-2 variants. However, the inventors found a significant reduction in neutralization against B.1.617.1 and B.1.617.2 compared to WT (FIG. 1c). Although antibody titers were lower against the VOCs and VUMs, these data indicate that serum antibodies elicited by natural WT infection were able to neutralize B.1.1.7, P.1 and WT virus equally, while most donors lost neutralizing potential against B.1.617-lineage viruses.

### 2. Generation of Mabs Against Distinct Domains of the Sars-Cov-2 Spike

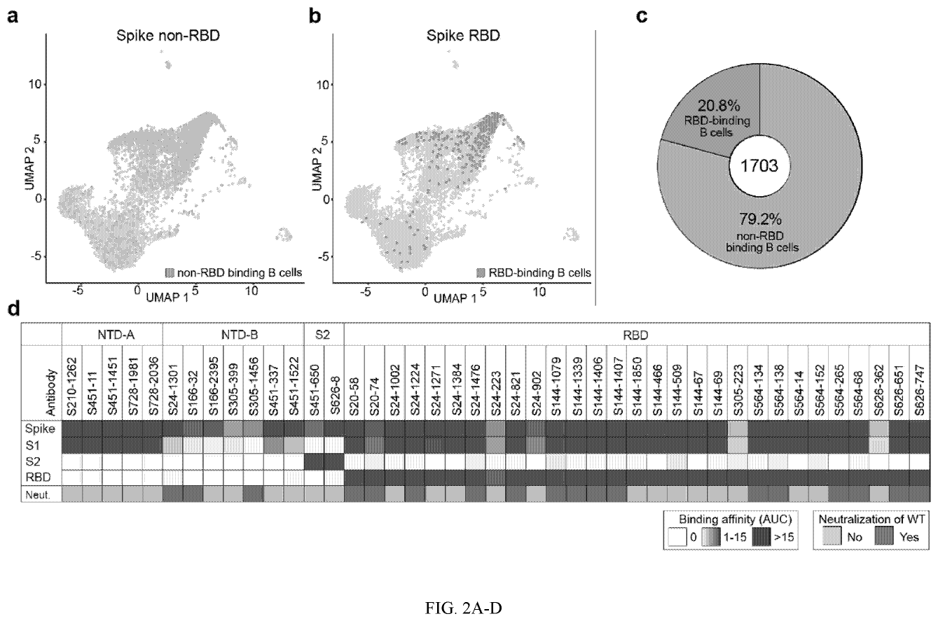

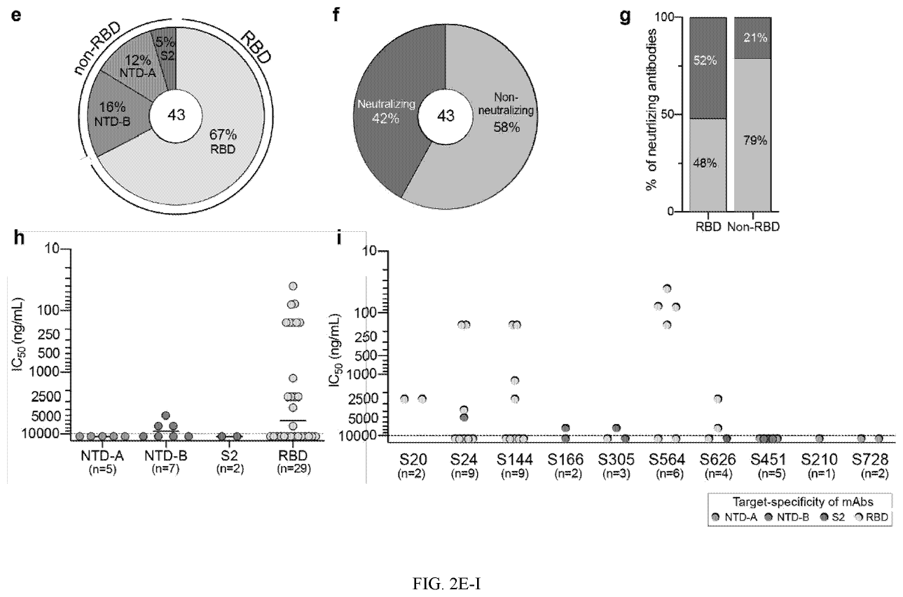

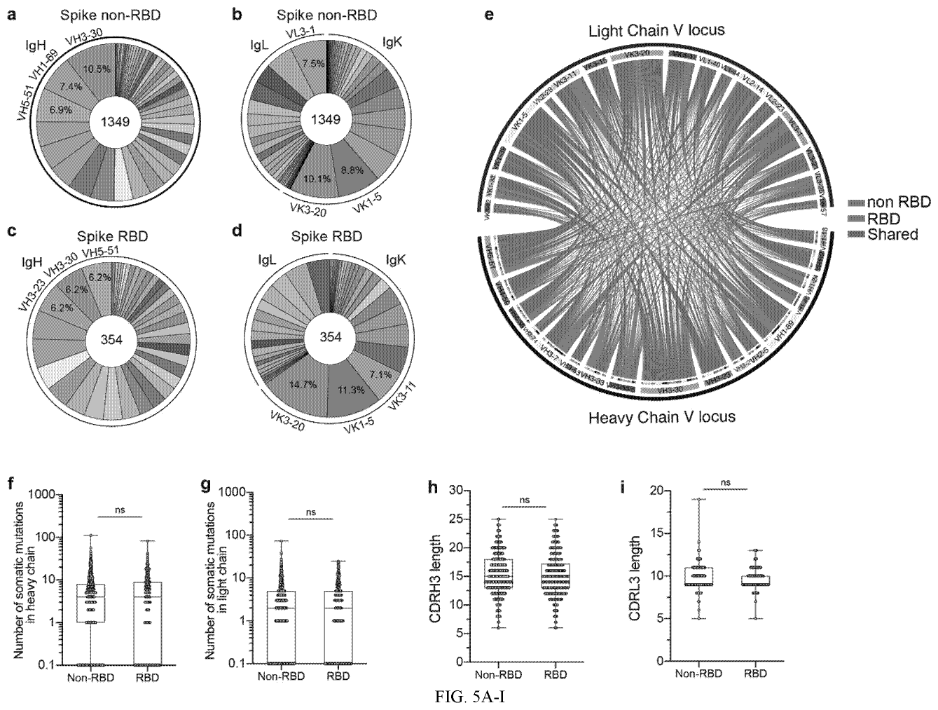

The inventors next sought to determine the specificities of antibodies that could cross-neutralize these viral variants by generating mAbs from spike-binding B cells isolated from 10 convalescent subjects collected between April and July of 2020^{26, 27}. The inventors sort-purified B cells binding to spike and/or RBD fluorophore- and oligo-conjugated probes, and performed single-cell RNA-sequencing and B cell receptor sequencing. As the antigen probes included a DNA oligonucleotide sequence, the inventors were able to track the antigen-specificity of isolated B cells. In total, the inventors obtained 1,703 paired immunoglobulin heavy and light chains from non-RBD- and RBD-binding B cells specific for the spike. Overall, the percentage of spike non-RBD-binding B cells was 4-fold higher than RBD-binding B cells (FIG. 2a-c), indicating that natural WT infection preferentially induced B cell response toward epitopes on the spike outside of the RBD28.29. Overall, B cells targeting the RBD or epitopes outside of the RBD utilized similar V (D) J genes, had overlapping heavy and light chain pairings, and possessed similar numbers of mutations and complementarity determining region 3 (CDR3) lengths (FIG. 5a-i).

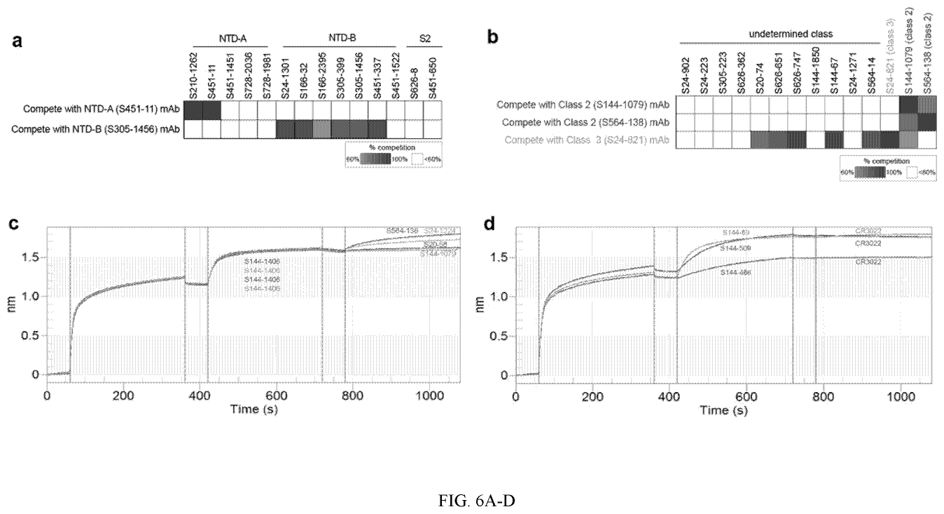

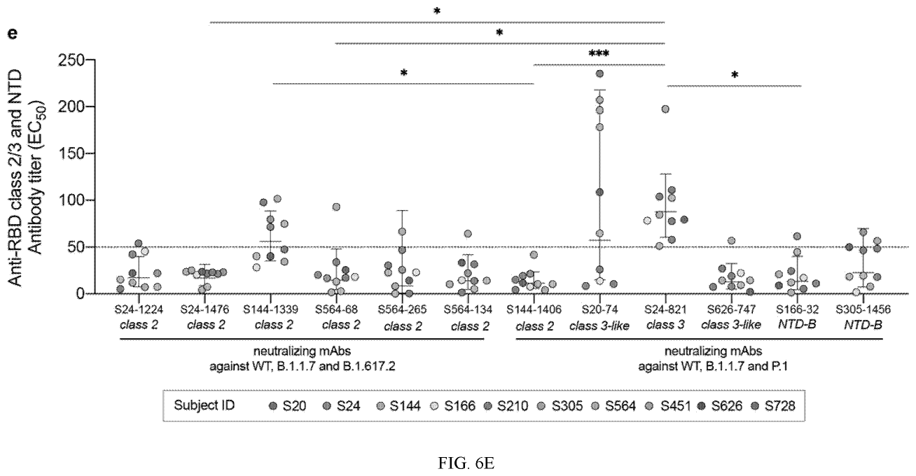

Based on the acquired antibody sequences and probe-binding intensities, the inventors generated 43 mAbs from all 10 donors specific to the WT spike protein (Supplementary Table 2). To investigate specific domain targeting, mAbs were tested for binding to the RBD and monomeric S1 and S2 recombinant spike antigens. Based on binding to these discrete antigens, spike-reactive mAbs were categorized into 4 groups: NTD-A-reactive mAbs (n=5) that bound strongly to S1 but not RBD, NTD-B-reactive mAbs (n=7) that weakly bind S1 but not RBD, S2-reactive mAbs (n=2), and RBD-reactive mAbs (n=29) (FIG. 2d-e). Additionally, NTD-A and NTD-B-classified antibodies targeted distinct epitopes as shown by competition ELISA (FIG. 6a). The inventors further determined whether antibodies with different binding specificities differ in their neutralization capacity against WT SARS-CoV-2. Of the 43 mAbs, 18 (42%) were neutralizing. Notably, only mAbs binding the RBD and NTD-B were neutralizing, whereas all mAbs binding NTD-A and S2 were non-neutralizing (FIG. 2d-f). Moreover, 52% of RBD-targeting mAbs were neutralizing, with eight mAbs being potently neutralizing antibodies (50% inhibitory concentration, IC50, of <500 ng/ml), and three out of seven NTD-B mAbs having moderate neutralization potency (5,000-7,500 ng/ml) (FIG. 2g-h). Of the 10 convalescent donors, seven had at least one neutralizing mAb among the antibodies cloned for this study, although the potencies of the mAbs varied by donor (FIG. 2i). Together, these data reveal that mAbs against the RBD are the predominate source of neutralizing antibodies induced by WT SARS-CoV-2 infection.

### 3. Binding and Neutralizing Breadth of Non-RBD Spike Antibodies

To understand the effects of viral variants on mAb binding to epitopes on the spike outside of the RBD, the inventors tested non-RBD-targeting mAbs for binding to a panel of SARS-CoV-2 variants, including D614G and the emerging variants B.1.1.7, B.1.351, P.1, B.1.617.2, B.1.526 and

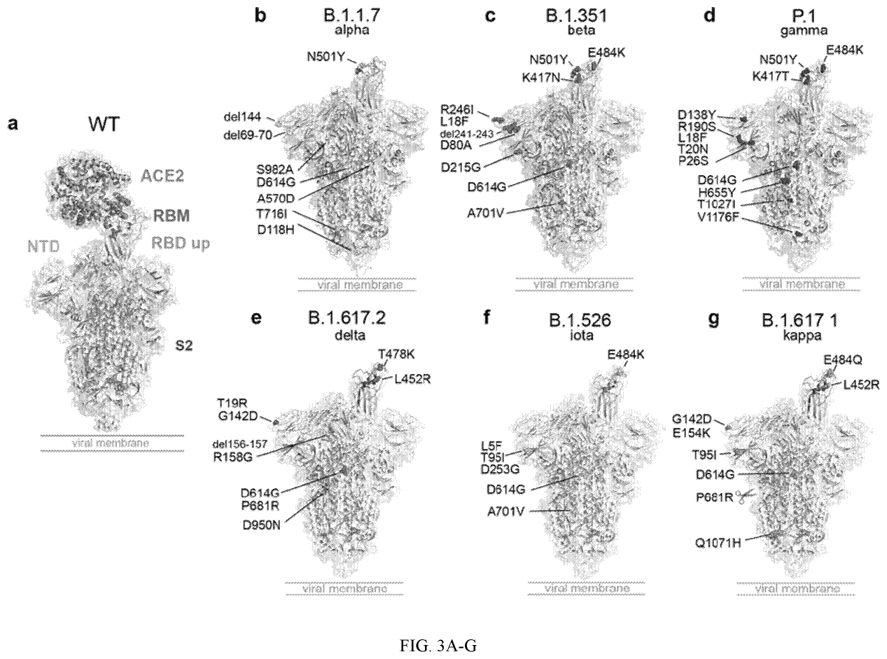

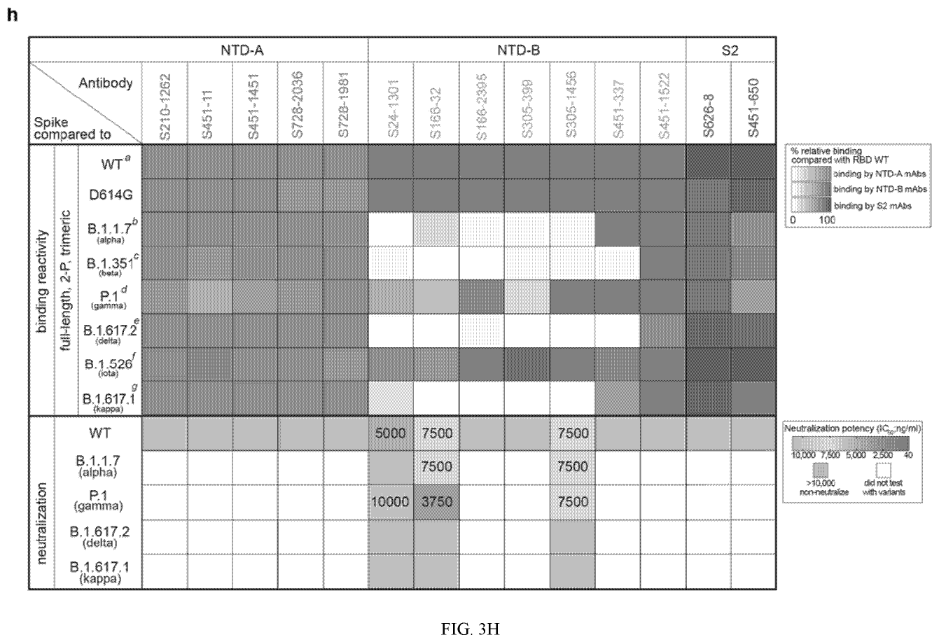

B.1.617.1 (FIG. 3a-g). All non-RBD spike-reactive antibodies showed similar binding to the D614G spike. Furthermore, all mAbs targeting NTD-A and S2 maintained similar binding to the spike of the B.1.1.7, B.1.351, P.1, B.1.617.2, B.1.526 and B.1.617.1 variants (FIG. 3h). Although mAbs against NTD-A and S2 retain binding to VOCs, they are non-neutralizing, implying that NTD-A- and S2-reactive antibodies may provide limited immune pressure to mutate these epitopes. Of interest, NTD-B mAbs showed significantly reduced binding to the spike of B.1.1.7, B.1.351, B.1.617.2 and B.1.617.1 while showing similar binding to B.1.526, and a minor reduction in binding to the spike of P.1 (FIG. 3h). Two of the three neutralizing NTD-B binding mAbs (S166-32 and S305-1456), which were isolated from two different subjects, retained neutralization potential against B.1.1.7 and P.1 at moderate neutralizing potency (FIG. 3h). The third neutralizing NTD-B-binding mAb (S24-1301) also had moderate neutralizing potency against the WT strain with weak cross-neutralization activity against the P.1 variant and no neutralization activity against B.1.1.7, consistent with its binding profile (FIG. 3h). However, all three neutralizing NTD-B mAbs failed to neutralize B.1.617.1 and B.1.617.2. Together, our data indicate that antibodies against NTD-B show cross-neutralization capacity and thus may provide protection against some emerging VOCs, such as B.1.1.7 and P.1. However, antibodies targeting the NTD-B epitope may be driving spike evolution, particularly the mutations and deletions found within B.1.1.7, B.1.351, B.1.617.1 and B.1.617.2, leaving the future of this epitope as a reliable target for cross-reactive antibodies uncertain.

4. A Subset of RBD-Binding mAbs Retain Neutralization Activity Against VOCs

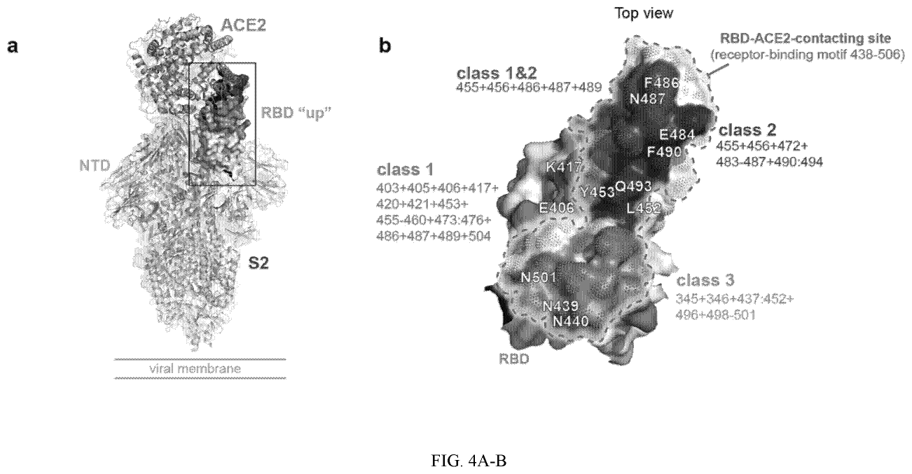

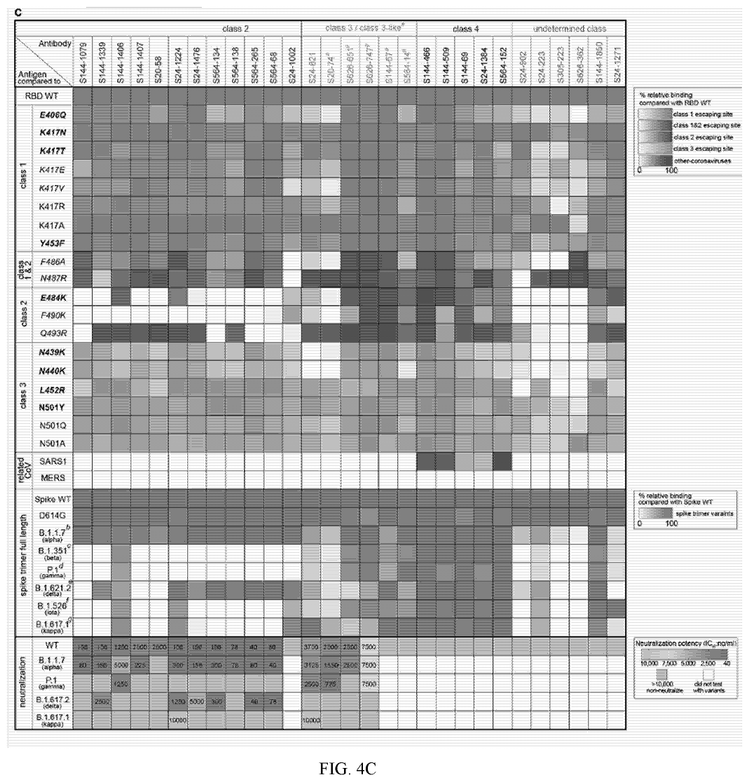

Viral escape mutations occurring within the RBD may result in reduction in neutralization capacity of RBD-targeting antibodies³⁰⁻³². To understand the impacts of RBD mutations on mAb binding, the inventors tested RBD-targeting mAbs for binding to RBD mutants that possessed a single mutation found in circulating SARS-CoV-2 VOCs, VOIs, VUMs or artificial mutants at key contact residues of the RBD-ACE2 interaction³⁰⁻³⁵, as well as full-length spike constructs containing multiple mutations in the RBD (Supplementary Table 3). In addition, the inventors tested mAb binding to the RBDs of SARS-CoV-1 and Middle Eastern Respiratory Syndrome (MERS)-CoV to investigate cross-reactivity to other coronaviruses. Notably, RBD-binding mAbs have been classified into four classes, classes 1-4 or receptor binding site (RBS) A-D, based on structural analysis and antibody binding features^{36, 37}. More recently, classification of four key antigenic regions of the RBD can also be defined by determining the loss of binding to RBD mutants (class 1-3 epitopes) or binding to cryptic epitopes on the RBD that are conserved across SARS-CoV-1 and MERS-CoV RBDs (class 4 epitope, FIG. 4a-b) 30.38. Based on the binding profiles of class 1˜4 binding mAbs, the inventors were able to segregate 23 out of 29 mAbs into one of the four classes (FIG. 4c and Supplementary FIG. 2b). Notably, no class 1 mAbs were found and six mAbs could not be classified as they either lost binding to multiple mutant classes or bound equally to all RBD mutants but did not bind to SARS-CoV-1 or MERS-CoV.

Class 2 RBD-binding mAbs showed reduced binding to at least one of the RBD class 2 single escape mutants, notably E484K and F490K, and the majority of these mAbs lost binding to the RBD mutants found in the B.1.351, P.1, B.1.526 and B.1.617.1 (FIG. 4c). Of the 12 class 2 mAbs, 11 were potently neutralizing against WT SARS-CoV-2. Of the neutralizing class 2 mAbs, all but one neutralized B.1.1.7 at concentrations comparable to neutralization of the WT strain. By contrast, six neutralized B.1.617.2 at lower potency compared to WT and B.1.1.7. Seven of the class 2 mAbs retained their neutralization activity against at least two VOCs (FIG. 4c). Of note, 10 out of 11 neutralizing class 2 mAbs were unable to neutralize the variants that harbored a mutation at E484, P.1 and B.1.617.1. This is in line with previous studies, which have shown that the E484K and E484Q mutations are the key escaping residue responsible for neutralization resistance by P.1, P.2, B.1.351 and B.1.617.1 VOCs^{2, 4, 39}. Of greatest interest, S144-1406, which retained binding to E484K and to all spike variants, neutralized B.1.1.7 and P.1 variants with high neutralization potency. Similar to another E484K-binder, S24-1224 neutralized three out of four VOCs tested, including B.1.617.1 (FIG. 4c). These data indicate that some class 2 antibodies can cross-neutralize VOCs. Additionally, the epitope targeted by S144-1406 partially overlapped with S24-1224 and other class 2 mAbs that failed to neutralize P.1 and B.1.617.1 (FIG. 6c), suggesting class 2 mAbs target similar but slightly different RBD epitopes.

Only one mAb (S24-821) specifically lost binding to the class 3 mutants, particularly to N439K and N440K, which are associated with circulating SARS-CoV-2 variants^{35, 40} and have been reported as in vitro escape sites for class 3 epitope-binding mAbs^{30, 31, 35} (FIG. 4b and Supplementary Table 3). Moreover, the inventors classified five more mAbs as class 3-like as they strongly competed for RBD binding with S24-821 but did not compete with class 2 mAbs (FIG. 6b). Importantly, all class 3 and class 3-like mAbs maintained binding to L452R, another mutation associated with class 3 antibodies that is present in B.1.427/B.1.429^{19, 41} and B.1.617 variants²⁰ (FIG. 4c). However, there was 2-3-fold reduction of class 3 and class 3-like mAbs in binding against B.1.617.2 which carry T487K and L452R substitutions in RBD region. Of the four neutralizing class 3 and class 3-like mAbs, all four retained neutralization activity against B.1.1.7 and three were neutralizing against P.1 (FIG. 4b). In contrast to class 2 mAbs, B.1.617.2 was resistant to all class 3-neutralizing mAbs. Only one mAb (S24-821) retained modest neutralization potency to B.1.617.1, indicating antibodies binding class 3 epitopes could neutralize some VOCs even though they bound L452R single mutation and all spike variants.

All of the mAbs that were categorized into class 4 (n=5) maintained binding to all RBD mutants and spike variants and displayed cross-reactivity to the SARS-CoV-1 RBD. However, all class 4 mAbs were non-neutralizing against WT virus, suggesting antibodies against this epitope are likely not strong drivers of antigenic drift. Notably, three antibodies in the class 4 group utilized the same heavy chain gene, VH5-51, as CR3022 and competed with CR3022 for binding to the RBD, indicating the class 4 antibodies in our study likely target the same or a similar epitope as CR3022 (Supplementary Table 2 and FIG. 6d). This is consistent with a previous study showing CR3022 cross-reacts with SARS-CoV-1, suggesting class 4 antibodies are common across subjects and studies 17.30.

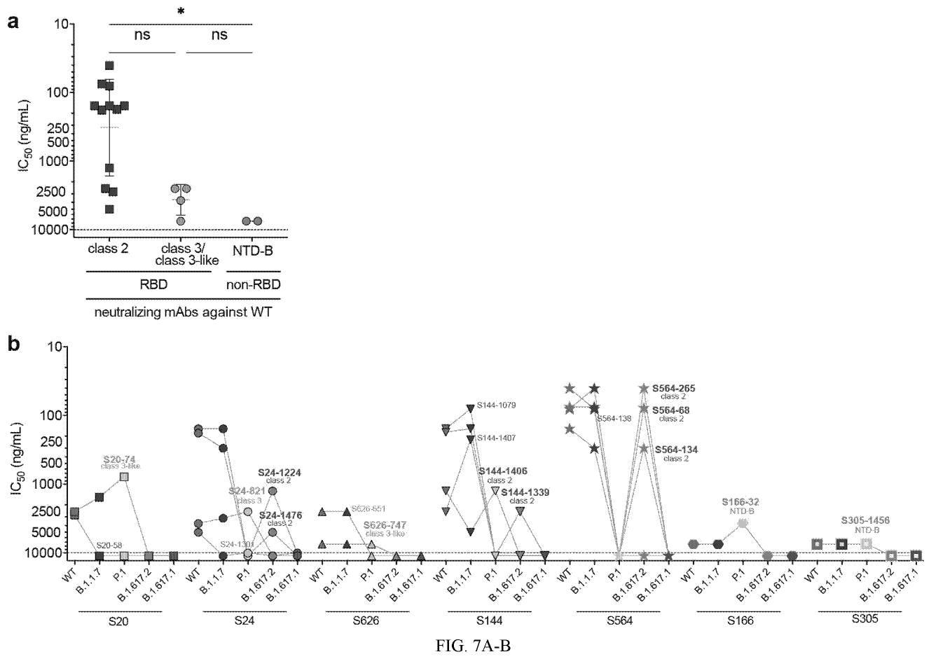

With the classification of mAbs against distinct epitopes, the inventors next tested the relative abundance of serum antibodies against these distinct epitopes of the RBD and NTD by performing competition assays. Notably, donors had significantly higher titers of serum antibodies targeting class 3 (S24-821) and class 3-like (S20-74) epitopes, whereas subjects largely had undetectable titers against class 2 and NTD-B epitopes, suggesting WT SARS-CoV-2 infection predominately induces polyclonal antibodies targeting RBD class 3 epitopes that can neutralize emerging VOCs B.1.1.7 and P.1. (FIG. 6e). These data are consistent with the observed anti-B.1.1.7 and anti-P.1 serum neutralizing titers shown in FIG. 1c, suggesting the retention of serum neutralization activity could be due to abundant class 3 antibody responses. Loss of neutralization capabilities to B.1.617-lineage viruses may be due to insufficient levels of class 2 serum antibodies. A comparison of the neutralization capabilities of mAbs targeting different epitopes revealed class 2 RBD-reactive mAbs were the most potently neutralizing followed by mAbs targeting class 3 RBD epitopes and NTD-B (FIG. 7a). It is important to note that none of neutralizing mAbs induced by natural WT infection were able to neutralize all emerging SARS-CoV-2 variants. Nonetheless, the inventors identified at least one mAb that could neutralize each VOC, suggesting the convalescent donors generated a diverse cross-neutralizing antibody response (FIG. 7b). Therefore, antibodies targeting multiple epitopes on the spike are a valuable source of neutralizing antibodies against emerging VOCs. Additionally, the inventors found majority of antibodies isolated from donors who had high antibody titers exhibited lower neutralizing potency than antibodies derived from donors who had lower serological titers and less severity (FIG. 7b and Supplementary Table 1). However, there was no difference between high and low responders in generating of cross-neutralizing antibodies against VOCs and VUMs. Moreover, the cross-neutralizing RBD-targeting mAbs used V (D) J gene features similar to other published RBD-binding mAbs (Supplementary Table 3)+2-++. However, the mAbs in our studies utilized distinct heavy and light chain pairings, indicating these clones are not public with other known neutralizing SARS-CoV-2 antibodies. Despite this, the data indicate that cross-neutralizing antibodies use a diverse antibody repertoire against multiple distinct epitopes. Therefore, driving a polyclonal antibody response against these three epitopes may provide cross-neutralizing protection against existing and future variants.

### B. Discussion

This study shows WT SARS-CoV-2-convalescent individuals possess antibodies that can effectively cross-neutralize against emerging VOCs, with cross-neutralizing antibodies targeting multiple epitopes of the spike protein. In total, the inventors identified 12 mAbs that potently neutralize current circulating VOCs, including B.1.1.7, the alpha variant that has been reported to be more infectious^{8, 19}, P.1, the gamma variant that partially escapes both natural and vaccine-induced humoral immunity^{2, 12, 45}, and B.1.617.2, the delta variant that is more transmissible than the alpha variant and has led to a surge of more hospitalizations in India and can evade partial immunity induced by one vaccine dose^{4, 15, 23}. Convalescent subjects in our cohort had sufficient serum titers to neutralize both B.1.1.7 and P.1 but not B.1.617, suggesting that the cross-neutralizing mAbs identified in this study may play an important role in polyclonal neutralization for some of VOCs.

Using high-throughput antigen probing at the single B cell level, the inventors found that B cells isolated from convalescent subjects largely targeted non-RBD epitopes rather than potently neutralizing epitopes on the RBD. Similarly, mRNA vaccines also largely induce antibodies against non-neutralizing epitopes, suggesting epitopes outside of the RBD are immunodominant⁴⁶. Despite this, vaccination has been shown to induce cross-neutralizing antibodies¹, suggesting both natural WT infection and currently approved vaccines can elicit protective humoral immunity against emerging variants. As the inventors identified 12 antibodies cross-neutralizing to VOCs derived from seven different convalescent COVID-19 donors, these study suggests most people generate a cross-neutralizing antibody response. Notably, these antibodies largely target three distinct epitopes, including two sites on the RBD and the one on the NTD. Several recent studies have demonstrated that antibodies against the NTD and S2 are neutralizing⁴⁷⁻⁴⁹. Although the anti-S2 mAbs identified in our study were non-neutralizing, S2-binding antibodies exhibit broad reactivity with spike proteins from SARS-CoV-2 variants, related beta coronaviruses such as SARS-CoV-1 and MERS-CoV, and distantly related endemic coronaviruses. Moreover, anti-spike serum antibodies can mediate protection via Fc-mediated functions, suggesting a combination of neutralizing antibodies and polyfunctional antibodies will provide optimal protection against infection with variants of SARS-CoV-250.

This study also showed that anti-RBD mAbs are primarily class 2 mAbs, consistent with other reports^{30, 37, 43, 51}. The majority of class 2 mAbs retained their neutralization activity against B.1.1.7 and B.1.617.2, but were largely non-neutralizing against P.1, suggesting class 2 mAbs may have driven the evolution of P.1 mutants. In contrast, neutralizing class 3 mAbs retained their neutralization activity against both B.1.1.7 and P.1, but did not neutralize the B.1.617 variants. Notably, none of the neutralizing mAbs could cross-neutralize B.1.1.7, P.1 and B.1.617.2, the most prevalent VOCs at this time. Therefore, vaccination approaches to increase affinity and frequencies of antibodies to the S1 domain may enhance the breadth of protection against emerging SARS-CoV-2 VOCs, including epitopes on the RBD and NTD. It is likely that targeting multiple epitopes will provide optimal protection so as to avoid generating escape mutants that can evade antibodies against any one epitope. Moreover, vaccinating previously infected subjects has been shown to substantially improve neutralization titers³ and may allow for refinement of memory B cells against neutralizing epitopes.

In conclusion, this study shows SARS-CoV-2 infection induces cross-neutralizing immunity against circulating VOCs, which is likely attributed to polyclonal antibodies targeting multiple epitopes of the spike protein. This work emphasizes the need for the induction of cross-neutralizing antibodies that bind distinct sites on the spike with various mechanisms that can synergize to provide protection against SARS-CoV-2 variants as well as limit the virus from escaping any single antibody target.

### C. Materials & Methods

### 1. Study Cohort and Spike-Specific B Cells Sorting

All studies were performed with the approval of the University of Chicago institutional review board IRB20-0523 and University of Chicago, University of Wisconsin-Madison. Informed consent was obtained after the research applications and possible consequences of the studies were disclosed to study subjects. The details of PBMC collection from leukoreduction filters were described elsewhere²⁷. For spike-specific B cells sorting, PBMC were thawed in 37° C. water bath and B cells were enriched using human pan B cell EasySep™ enrichment kit (STEMCELL). B cells were stained with anti-CD19-PE-Cy7 (Biolegend) and anti-CD3-BV510 (BD Biosciences) and antigen probes (PE) for 30 minutes on ice in 1×PBS supplemented with 0.2% BSA and 2 mM Pierce Biotin. Probe generation was performed as previously described²⁷. Cells were subsequently washed with 1×PBS with 0.2% BSA and stained with Live/Dead BV510 (Thermo Fisher) in 1×PBS for 15 minutes. Cells were washed again and re-suspended at a maximum of 4 million cells/mL in 1×PBS supplemented with 0.2% BSA and 2 mM Pierce Biotin for downstream cell sorting using the MACSQuantTyto cartridge sorting platform (Miltenyi). Viable/CD19+/antigen-PE+ cells were sorted as probe positive. Cells were then collected from the cartridge sorting chamber and used for downstream processing with the chromium controller (10× Genomics).

### 2. Single-Cell RNA-Seq and B Cell Receptor Sequencing

The human B cell V (D) J, 5′ gene expression, feature barcode libraries were prepared according to manufacturer's instructions. Libraries were pooled and sequenced using an Illumina NextSeq 550 or an Illumina NextSeq 500 at the University of Chicago. Cell Ranger (version 3.0.2) was used to perform raw sequencing processing, sample de-multiplexing, barcode processing, single-cell 5′ transcripts counting and B cell receptor repertoire sequences assembly. The reference genome assembly for transcriptome is GRCh38-1.2.0, and reference genome assembly for V (D) J is cellranger-vdj-GRCh38-alts-ensembl-2.0.0. The data obtained from Cell Ranger were subsequently performed downstream analysis using Seurat toolkit (version 3.2.0, an R package, for transcriptome, cell surface protein and antigen probe analysis) 52 and IgBlast (version 1.15) for immunoglobulin gene analysis⁵³. Cell quality control (QC), normalization, data scaling, and linear dimensional reduction, clustering, differential expression analysis, batch effects correction, and data visualization were processed using Seurat (version 3.2.0). The QC of cells were performed further to exclude cells with less than 200 and more then 2500 detected genes and cells expressing high percentage of mitochondrial genes. Transcriptome RNA data was analyzed using conventional log normalization. The inventors performed principal component analysis (PCA) and used the top 15 principal components (PCs) in linear dimensional reduction and clustering. Only filtered, high-quality cells were clustered in this analysis using Louvain algorithm implemented in Seurat under the resolution of 0.6 for clustering. Batch effects across different datasets were normalized using an Anchor method implemented in Seurat.

### 3. Monoclonal Antibody Production

B cells were selected for mAb generation based on antigen probe intensity visualized by JMPPro 15, as previous described²⁷. Antibody heavy and light chain genes obtained by 10× Genomics V(D)J sequencing analysis were synthesized by Integrated DNA Technologies. The synthesized fragments for heavy and light chain with 5′ and 3′ Gibson overhangs were then cloned into human IgG1 and human kappa or lambda light chain expression vectors by Gibson assembly as previously described⁵⁴. The heavy and light chains of a corresponding mAb were co-transfected into HEK293T cells. After 4 days, secreted mAbs in the medium supernatant were harvested and purified using protein A agarose beads (Thermo Fisher).

### 4. Recombinant Proteins

The recombinant WT SARS-CoV-2 full-length (FL) spike, D614G FL spike, WT RBD, K417T/R/A RBD, N501Q/A RBD, and SARS-CoV-1 RBD and MERS-CoV were generated in-house either by using gBlock fragment synthesized by Integrated DNA Technologies or by performing single-site mutagenesis, and expressed by Expi293F cells (Thermo Fisher). The recombinant FL spikes derived from variants of B.1.1.7, B.1.351, P.1, B.1.617.2, B.1.526, and were kindly provided by Dr. Noah Sather laboratory at Seattle Children's Research Institute. The recombinant RBD found in VOCs, B.1.351 or P.1 variants, and RBD with single mutation or multiple mutations (N439: Y453F, E406Q, K417E, K417V, Y453F, F486A, N487R, F490K, Q493R, N439K, N440K, N501Y) were generously provided from the Krammer laboratory at Icahn School of Medicine at Mount Sinai. The recombinant S1 and S2 subunit, and RBD with single mutation of K417N, E484K and L452R were obtained from Sino Biological. The protein sequences and resources for each antigen are listed in Extended Data Table 3.

### 5. Virus Neutralization Assay

Virus neutralization assays were performed with different variants of SARS-CoV-2 on Vero E6/TMPRSS2 (Extended Data Table 4). Virus (˜100 plaque-forming units) was incubated with an equal volume of two-fold diluted of serum or mAbs for 1 hour. Plasma samples were diluted in calcium free media, while antibodies were diluted in growth media. In addition, plasma was heat treated for 30 minutes at 37° C. prior to use. The antibody/virus mixture was added to confluent Vero E6/TMPRSS2 cells that were plated at 30,000 cells per well the day prior in 96-well plates. The cells were incubated for 3 days at 37° C. and then fixed and stained with 20% methanol and crystal violet solution. Virus neutralization titers were determined as the reciprocal of the highest serum dilution that completely prevented cytopathic effects. The 50% inhibitory concentrations for mAbs (IC50) was determined using log (inhibitor) versus normalized response (variable slope) performed by Prism (Graphpad Version 9.0). All plasma and mAbs were tested in duplicate and each experiment was performed twice.

### 6. Enzyme-Linked Immunosorbent Assay (ELISA)

High-protein binding microtiter plates (Costar) were coated with 50 μl of recombinant proteins (either full-length spike or RBD) at 2 μg/ml in 1×PBS solution overnight at 4° C. The plates were washed 3 times the next day with 1×PBS supplemented with 0.05% Tween 20 and blocked with 175 μl of 1×PBS containing 20% FBS for 1 hour at 37° C. MAbs were serially diluted 1:3 starting at 10 μg/ml and incubated for 1 hour at 37° C. The plates were then washed 3 times and incubated with horseradish peroxidase (HRP)-conjugated goat anti-human IgG antibody (Jackson ImmunoResearch) diluted 1:1000 for 1 hour at 37° C., and plates were subsequently developed with Super AquaBlue ELISA substrate (eBioscience). Absorbance was measured at 405 nm on a microplate spectrophotometer (Bio-Rad). To standardize the assays, control antibodies with known binding characteristics were included on each plate and the plates were developed when the absorbance of the control reached 3.0 OD405 units. All mAbs were tested in duplicate and each experiment was performed twice.

### 7. Competition ELISAs

To determine the classification of certain mAbs, competition ELISAs were carried out using the mAbs with known epitope binding property as competitor mAbs. The competitor mAbs were biotinylated overnight at 4° C. with EZ-Link™ Sulfo-NHS-Biotin (Thermo Scientific). The excess free biotin of biotinylated mAbs were removed by 7k MWCO Zeba™ spin desalted columns (Thermo Scientific). Plates were coated with 50 μl of 2 μg/ml RBD antigen overnight at 4° C. After 1 hour of blocking the plates with PBS 20% FBS, the 2-fold dilution of undetermined classmAbs or serum were added (starting at 20 μg/ml of mAbs and 1:50 of serum) into coated well. After incubated for 2 hours at room temperature, biotinylated competitor mAb was added at a concentration of 2× K_{d} and incubated another 2 hours at room temperature together with mAbs or serum that were previously added. The plates were washed 3 times and incubated with 100 μl HRP-conjugated streptavidin (Southern Biotech) dilution of 1:1000 for 1 hour at 37° C. The plates were developed with Super AquaBlue ELISA substrate (eBioscience). To standardize the assays, competitor biotinylated mAb was added in well that without any competing mAbs or serum as control well. The data were recorded when the absorbance of the control well reached 1 to 1.5 OD405 units. All mAbs were tested in duplicate and each experiment was performed twice. The percent competition was then calculated by dividing a sample's observe OD by the OD reached by the positive control, subtracting this value from 1, and multiplying by 100. For the serum data, ODs were log transformed and analyzed by non-linear regression to determine EC50 values using Prism software (Graphpad Version 9.0).

### 8. Biolayer Interferometry (BLI)

To determine the classification of certain mAbs, competition assays were performed using the mAbs with known epitope binding property as competitor mAbs with mAb binding unknown epitopes using BLI with a Octet K2 instrument (Forte Bio). The RBD of SARS-CoV-2 was biotinylated, desalted and loaded at a concentration of 10 μg/ml onto streptavidin probes for 300 seconds followed by PBS for 60 seconds. The probe was moved to associate with mAbs of interest (10 μg/ml) for 300 seconds followed by PBS for 60 seconds and then associations with control mAbs (10 μg/mL) for 300 seconds. The final volume for all the solutions was 200 ml/well. All of the assays were performed with PBS buffer at 30° C.

### 9. SARS-CoV-2 Spike and RBD Protein Models

FL mutations were visualized on the WT spike protein (PDB: 7KJ2) using PyMOL (Schrödinger). The model of RBD mutations and RBD classes were visualized on the WT RBD protein (PDB: 7KDL) using PyMOL (Schrödinger). The models were further processed by Adobe Illustrator 2021 and Adobe Photoshop.

### 10. Statistical Analysis

All statistical analyses were performed using Prism software (Graphpad Version 9.0). Sample sizes (n) for the number of mAbs tested are indicated in corresponding figures or in the center of pie graphs. Number of biological repeats for experiments and specific tests for statistical significance used are indicated in the corresponding figure legends. P values less than or equal to 0.05 were considered significant. * P≤0.05, ** P≤0.01, *** P≤0.001, **** P<0.0001.

### D. Tables

SUPPLEMENTARY TABLE 1
COVID-19 convalescent subjects. Responder group and
Severity were categorized by previous study²⁶.

| | | | | | Symptom | | |
|---|---|---|---|---|---|---|---|
| | | | SARS- | Duration of | start to | | |
| Subject | | | CoV-2 | symptoms | donation | Responder | Severity |
| ID | Age | Sex | PCR Test | (days) | (days) | Category26 | Category26 |
| 24 | 34 | M | Mar. 23, 2020 | 12 | 41 | High | Severe |
| 20 | 31 | M | Mar. 31, 2020 | 19 | 48 | High | Critical |
| 564 | 24 | F | Mar. 19, 2020 | 32 | 60 | Low | Severe |
| 144 | 56 | M | Mar. 16, 2020 | 23 | 54 | Low | Moderate |
| 305 | 43 | F | Apr. 17, 2020 | 4 | 47 | Low | Moderate |
| 166 | 42 | F | Mar. 25, 2020 | 17 | 55 | Low | Moderate |
| 210 | 47 | M | Apr. 4, 2020 | 7 | 41 | Low | Moderate |
| 451 | 46 | M | Apr. 4, 2020 | 11 | 49 | High | Severe |
| | | | | | | | (hospitalized) |
| 626 | 44 | M | Mar. 31, 2020 | 19 | 56 | High | Moderate |
| 728 | 62 | F | Mar. 15, 2020 | 53 | 130 | High | Severe |

SUPPLEMENTARY TABLE 2
Characteristics of SARS-CoV-2 spike binding mAbs. Cross-neutralizing
mAbs against WT, B.1.1.7 and P.1 or B.1.617.2 are bolded.

| mAb | Epitope | | | # VH | #VL | CDRH3 | CDRL3 |
|---|---|---|---|---|---|---|---|
| ID | specificity | VH gene | VL gene | SHM | SHM | length | length |
| S20-58 | Spike RBD | IGHV4- | IGKV2- | 5 | 2 | 15 | 9 |
| | Class 2 | 30-4*08 | 24*01 | | | | |
| S20-74 | Spike RBD | IGHV4- | IGLV2- | 7 | 3 | 15 | 11 |
| | Class 3-like | 59*11 | 8*01 | | | | |
| S24- | Spike RBD | IGHV2- | IGLV2- | 1 | 3 | 11 | 11 |
| 223 | undetermined | 5*02 | 14*01 | | | | |
| S24- | Spike RBD | IGHV2- | IGKV1- | 3 | 0 | 16 | 9 |
| 821 | Class 3 | 70*15 | 5*03 | | | | |
| S24- | Spike RBD | IGHV1- | IGLV7- | 0 | 0 | 15 | 8 |
| 902 | undetermined | 69*04 | 46*01 | | | | |
| S24- | Spike RBD | IGHV3- | IGKV1- | 4 | 5 | 25 | 9 |
| 1002 | Class 2 | 30-3*01 | 13*02 | | | | |
| S24- | Spike RBD | IGHV1- | IGLV1- | 8 | 7 | 20 | 11 |
| 1224 | Class 2 | 46*01 | 40*01 | | | | |
| S24- | Spike RBD | IGHV3- | IGLV3- | 7 | 6 | 22 | 9 |
| 1271 | undetermined | 66*01 | 1*01 | | | | |
| S24- | Spike | IGHV1- | IGLV10- | 6 | 4 | 18 | 11 |
| 1301 | NTD-B | 24*01 | 54*01 | | | | |
| S24- | Spike RBD | IGHV3- | IGLV3- | 3 | 4 | 17 | 12 |
| 1384 | Class 4 | 48*04 | 21*02 | | | | |
| S24- | Spike RBD | IGHV3- | IGKV3- | 3 | 0 | 18 | 8 |
| 1476 | Class 2 | 49*03 | 15*01 | | | | |
| S144- | Spike RBD | IGHV5- | IGLV1- | 8 | 5 | 17 | 12 |
| 67 | Class 3-like | 51*01 | 40*01 | | | | |
| S144- | Spike RBD | IGHV5- | IGKV1- | 3 | 3 | 11 | 8 |
| 69 | Class 4 | 51*01 | 5*01 | | | | |
| S144- | Spike RBD | IGHV5- | IGKV1- | 7 | 6 | 11 | 9 |
| 466 | Class 4 | 51*01 | 5*01 | | | | |
| S144- | Spike RBD | IGHV5- | IGKV1- | 3 | 1 | 12 | 9 |
| 509 | Class 4 | 51*01 | 5*01 | | | | |
| S144- | Spike RBD | IGHV1- | IGKV3- | 9 | 3 | 19 | 9 |
| 1079 | Class 2 | 69*02 | 20*01 | | | | |
| S144- | Spike RBD | IGHV1- | IGLV2- | 15 | 5 | 18 | 11 |
| 1339 | Class 2 | 2*06 | 14*01 | | | | |
| S144- | Spike RBD | IGHV1- | IGKV1- | 4 | 0 | 11 | 17 |
| 1406 | Class 2 | 3*01 | 5*01 | | | | |
| S144- | Spike RBD | IGHV1- | IGKV1- | 9 | 2 | 12 | 10 |
| 1407 | Class 2 | 69*02 | 5*01 | | | | |
| S144- | Spike RBD | IGHV3- | IGKV1- | 2 | 3 | 13 | 9 |
| 1850 | undetermined | 23*04 | 5*01 | | | | |
| S166- | Spike | IGHV3- | IGKV1- | 10 | 2 | 19 | 8 |
| 32 | NTD-B | 11*01 | 5*01 | | | | |
| S166- | Spike | IGHV4- | IGLV3- | 3 | 5 | 16 | 12 |
| 2395 | NTD-B | 4*07 | 21*02 | | | | |
| S210- | Spike | IGHV4- | IGLV4- | 11 | 4 | 10 | 9 |
| 1262 | NTD-A | 39*01 | 69*01 | | | | |
| S305- | Spike RBD | IGHV3- | IGKV3- | 18 | 8 | 6 | 9 |
| 223 | Class 2 | 33*06 | 11*01 | | | | |
| S305- | Spike RBD | IGHV1- | IGKV3- | 4 | 4 | 18 | 9 |
| 399 | undetermined | 24*01 | 15*01 | | | | |
| S305- | Spike | IGHV1- | IGKV3- | 3 | 3 | 20 | 9 |
| 1456 | NTD-B | 24*01 | 15*01 | | | | |
| S451- | Spike | IGHV3- | IGKV3D- | 8 | 3 | 15 | 8 |
| 11 | NTD-A | 23*01 | 20*01 | | | | |
| S451- | Spike | IGHV4- | IGKV3- | 10 | 1 | 15 | 10 |
| 337 | NTD-B | 59*01 | 20*01 | | | | |
| S451- | Spike S2 | IGHV3- | IGKV3- | 6 | 4 | 14 | 8 |
| 650 | | 30*01 | 20*01 | | | | |
| S451- | Spike | IGHV4- | IGLV2- | 9 | 5 | 14 | 10 |
| 1451 | NTD-A | 31*01 | 11*01 | | | | |
| S451- | Spike | IGHV2- | IGLV2- | 8 | 5 | 20 | 11 |
| 1522 | NTD-B | 26*01 | 14*01 | | | | |
| S564- | Spike RBD | IGHV3- | IGLV3- | 6 | 3 | 18 | 12 |
| 14 | Class 3-like | 7*01 | 21*04 | | | | |
| S564- | Spike RBD | IGHV1- | IGLV2- | 6 | 2 | 15 | 10 |
| 68 | Class 2 | 2*02 | 8*01 | | | | |
| S564- | Spike RBD | IGHV1- | IGLV2- | 2 | 6 | 15 | 10 |
| 134 | Class 2 | 2*02 | 8*01 | | | | |
| S564- | Spike RBD | IGHV1- | IGLV2- | 10 | 1 | 18 | 10 |
| 138 | Class 2 | 2*02 | 14*01 | | | | |
| S564- | Spike RBD | IGHV3- | IGKV1- | 4 | 4 | 20 | 10 |
| 152 | Class 4 | 33*06 | 33*01 | | | | |
| S564- | Spike RBD | IGHV1- | IGLV2- | 4 | 3 | 15 | 10 |
| 265 | Class 2 | 2*02 | 8*01 | | | | |
| S626-8 | Spike S2 | IGHV1- | IGLV3- | 7 | 5 | 24 | 12 |
| | | 8*01 | 19*01 | | | | |
| S626- | Spike RBD | IGHV3- | IGLV1- | 18 | 3 | 16 | 11 |
| 362 | undetermined | 48*01 | 40*01 | | | | |
| S626- | Spike RBD | IGHV1- | IGLV1- | 6 | 4 | 17 | 11 |
| 651 | Class 3-like | 69*04 | 40*01 | | | | |
| S626- | Spike RBD | IGHV3- | IGKV1- | 6 | 6 | 22 | 10 |
| 747 | Class 3-like | 9*01 | 33*01 | | | | |
| S728- | Spike | IGHV1- | IGKV3- | 17 | 3 | 16 | 11 |
| 1981 | NTD-A | 46*01 | 11*01 | | | | |
| S728- | Spike | IGHV1- | IGLV2- | 14 | 12 | 17 | 10 |
| 2036 | NTD-A | 2*02 | 23*02 | | | | |

SUPPLEMENTARY TABLE 3
Antigen information and source. VOC refers to variant
of concern and VUM refers to variant under monitoring.

| | | | | | Mutation | |
|---|---|---|---|---|---|---|
| Antigen | S1 NTD | RBD | S1 CTD | S2 | detected in | Source |
| Spike FL, 2-P, trimer | | | | | | |
| WT | — | — | — | — | — | In-house |
| D614G | — | — | D614G | — | VOC | In-house |
| B.1.1.7 (alpha) | H69del, | N501Y | A570D, | T716I, | VOC | Sather lab |
| | V70del, | | D614G, | S982A, | | |
| | Y144del | | P681H | D1118H | | |
| B.1.351 (beta) | L18F, | K417N, | D614G | A701V | VOC | Sather lab |
| | D80A, | E484K. | | | | |
| | D215G, | N501Y | | | | |
| | del241-243, | | | | | |
| | R246I | | | | | |
| P.1 (gamma) | L18F, | K417T, | D614G, | T1027I, | VOC | Sather lab |
| | T20N, | E484K, | H655Y | V1176F | | |
| | P26S, | N501Y | | | | |
| | D138Y, | | | | | |
| | R190S | | | | | |
| B.1.617.2 (delta) | T19R, | L452R, | D614G, | D950N | VOC | Sather lab |
| | G142D, | T478K, | P681R | | | |
| | del156-157, | | | | | |
| | R158G | | | | | |
| B.1.526 (iota) | L5F, T95I, | E484K | D614G | A701V | VUM | Sather lab |
| | D253G | | | | | |
| B.1.617.1 | T95I, | L452R, | D614G, | Q1071H | VUM | Sather lab |
| (kappa) | G142D, | E484Q | P681R | | | |
| | E154K | | | | | |
| S1 monomeric | | | | | | |
| WT | — | — | — | — | — | SinoBiological |
| S2 monomeric | | | | | | |
| WT | — | — | — | — | — | SinoBiological |
| RBD | | | | | | |
| WT | — | — | — | — | — | In-house |
| E406Q | — | E406Q | — | — | Circulating | Krammer lab |
| | | | | | variant, In vitro | |
| | | | | | escape | |
| K417N | — | K417N | — | — | VOC, In vitro | SinoBiological |
| (B.1.351) | | | | | escape | |
| K417T (P.1) | — | K417T | — | — | VOC, In vitro | In-house |
| | | | | | escape | |
| K417E | — | K417E | — | — | In vitro escape | Krammer lab |
| K417V | — | K417V | — | — | In vitro escape | Krammer lab |
| K417A | — | K417A | — | — | RBD-ACE2 | In-house |
| | | | | | contacting | |
| Y453F (B.1.427, | — | Y453F | — | — | VOC, In vitro | Krammer lab |
| B.1.429) | | | | | escape | |
| F486A | — | F486A | — | — | In vitro escape | Krammer lab |
| N487R | — | N487R | — | — | In vitro escape | Krammer lab |
| E484K (P.1, | — | E484K | — | — | VOC, In vitro | Krammer lab |
| B.1.526, | | | | | escape | |
| B.1.351, | | | | | | |
| B.1.1.318, | | | | | | |
| B.1.525, R.1, | | | | | | |
| B.1.526.2, B.1.1, | | | | | | |
| B.1.621, B.1, | | | | | | |
| B.1.1.7) | | | | | | |
| F490K | — | F490K | — | — | In vitro escape | Krammer lab |
| Q493R | — | Q493R | — | — | In vitro escape | Krammer lab |
| N439K | — | N439K | — | — | VOC, In vitro | Krammer lab |
| | | | | | escape | |
| N440K (B.1.36) | — | N440K | — | — | Circulating | Krammer lab |
| | | | | | variant, In vitro | |
| | | | | | escape | |
| L452R | — | L452R | — | — | VOC, In vitro | SinoBiological |
| (B.1.526.1, | | | | | | |
| B.1.429, | | | | | | |
| B.1.427, | | | | | escape | |
| B.1.617.2, B.1, | | | | | | |
| B.1.617.1, C.36, | | | | | | |
| A.2.5) | | | | | | |
| N501Y (B.1.1.7) | — | N501Y | — | — | VOC | Krammer lab |
| N501Q | — | N501Q | — | — | RBD-ACE2 | Krammer lab |
| | | | | | contacting | |
| N501A | — | N501A | — | — | RBD-ACE2 | Krammer lab |
| | | | | | contacting | |
| B.1.351 | | K417N, | | | VOC | Krammer lab |
| | | E484K, | | | | |
| | | N501Y | | | | |
| P.1 | K417T, | | | | VOC | Krammer lab |
| | E484K, | | | | | |
| | N501Y | | | | | |

| Other coronaviruses | | |
|---|---|---|
| SARS-CoV-1 RBD WT | — | In-house |
| MERS-CoV RBD WT | — | In-house |

SUPPLEMENTARY TABLE 4
SARS-CoV-2 virus information and source.

| Antigen | S1 NTD | RBD | S1 CTD | S2 | Source |
|---|---|---|---|---|---|
| WT | — | — | — | — | SARS-CoV-2/UT- |
| | | | | | NCGM02/Human/2020/Tokyo |
| | | | | | from BEI |
| B.1.1.7 | L5F, H69del, | N501Y | A570D, | T716I, | hCoV- |
| | V70del, | | D614G, | S982A, | 19/Japan/QHN001/2020 from |
| | Y144del | | P681H | D1118H | BEI |
| P.1 | L18F, T20N, | K417T, | D614G, | T1027I, | hCoV-19/Japan/TY7- |
| | P26S, D138Y, | E484K, | H655Y | V1176F | 501/2021 from BEI |
| | G181V, R190S | N501Y | | | |
| B.1.617.1 | G142D, E154K | L452R, | D614G, | Q1071H, | hCoV-19/USA/CA-Stanford- |
| | | E484Q | P681R | H1101D | 15_S02/2021 from BEI |
| B.1.617.2 | T19R, T95I, | L452R, | D614G, | D950N | hCoV-19/USA/WI-UW- |
| | G142D, E156G, | T478K | P681R | | 5250/2021 |
| | F157del, | | | | |
| | R158del | | | | |

### E. References

The following references and the references cited throughout the specification, to the extent that they provide exemplary procedural or other details supplementary to those set forth herein, are specifically incorporated herein by reference.
1. Baden, L. R. et al. Efficacy and Safety of the mRNA-1273 SARS-CoV-2 Vaccine. New Engl J Med 384, 403-416, doi: 10.1056/NEJMoa2035389 (2021).
2. Garcia-Beltran, W. F. et al. Multiple SARS-CoV-2 variants escape neutralization by vaccine-induced humoral immunity. Cell, doi: 10.1016/j.cell.2021.03.013 (2021).
3. Wang, Z. J. et al. mRNA vaccine-elicited antibodies to SARS-CoV-2 and circulating variants. Nature, doi: 10.1038/s41586-021-03324-6 (2021).
4. Wall, E. C. et al. Neutralising antibody activity against SARS-CoV-2 VOCs B. 1.617. 2 and B. 1.351 by BNT162b2 vaccination. The Lancet 397, 2331-2333 (2021).
5. Jiang, S. B., Hillyer, C. & Du, L. Y. Neutralizing Antibodies against SARS-CoV-2 and Other Human Coronaviruses. Trends Immunol 41, 355-359, doi: 10.1016/j.it.2020.03.007 (2020).
6. Korber, B. et al. Tracking Changes in SARS-CoV-2 Spike: Evidence that D614G Increases Infectivity of the COVID-19 Virus. Cell 182, 812-+, doi: 10.1016/j.cell.2020.06.043 (2020).
7. Leung, K., Shum, M. H., Leung, G. M., Lam, T. T. & Wu, J. T. Early transmissibility assessment of the N501Y mutant strains of SARS-CoV-2 in the United Kingdom, October to November 2020. Eurosurveillance 26, 2002106 (2021).
8. Liu, H. et al. The basis of a more contagious 501Y.V1 variant of SARS-CoV-2. Cell Res, doi: 10.1038/s41422-021-00496-8 (2021).
9. Volz, E. et al. Assessing transmissibility of SARS-CoV-2 lineage B. 1.1. 7 in England. Nature 593, 266-269 (2021).
10. Davies, N. G. et al. Estimated transmissibility and impact of SARS-CoV-2 lineage B. 1.1. 7 in England. Science 372 (2021).
11. Annavajhala, M. K. et al. A Novel SARS-CoV-2 Variant of Concern, B.1.526, Identified in New York. medRxiv, doi: 10.1101/2021.02.23.21252259 (2021).
12. Nonaka, C. K. V. et al. Genomic Evidence of SARS-CoV-2 Reinfection Involving E484K Spike Mutation, Brazil. Emerg Infect Dis 27, doi: 10.3201/eid2705.210191 (2021).
13. Tegally, H. et al. Sixteen novel lineages of SARS-CoV-2 in South Africa. Nat Med 27, 440-+, doi: 10.1038/s41591-021-01255-3 (2021).
14. Hirotsu, Y. & Omata, M. Detection of R.1 lineage severe acute respiratory syndrome coronavirus 2 (SARS-CoV-2) with spike protein W152L/E484K/G769V mutations in Japan. PLOS Pathog 17, e1009619, doi: 10.1371/journal.ppat. 1009619 (2021).
15. Edara, V.-V. et al. Infection and vaccine-induced neutralizing antibody responses to the SARS-CoV-2 B. 1.617. 1 variant. bioRxiv (2021).
16. Shang, J. et al. Structural basis of receptor recognition by SARS-CoV-2. Nature 581, 221-+, doi: 10.1038/s41586-020-2179-y (2020).
17. Yuan, M., Liu, H. J., Wu, N. I. C. & Wilson, I. A. Recognition of the SARS-CoV-2 receptor binding domain by neutralizing antibodies. Biochem Bioph Res Co 538, 192-203, doi: 10.1016/j.bbrc.2020.10.012 (2021).
18. Zhou, D. et al. Evidence of escape of SARS-CoV-2 variant B.1.351 from natural and vaccine-induced sera. Cell, doi: 10.1016/j.cell.2021.02.037 (2021).
19 Plante, J. A. et al. The variant gambit: COVID-19's next move. Cell Host Microbe 29, 508-515, doi: 10.1016/j.chom.2021.02.020 (2021).
20 Yadav, P. D. et al. Neutralization of variant under investigation B.1.617 with sera of BBV152 vaccinees. Clin Infect Dis, doi: 10.1093/cid/ciab411 (2021).
21. Singh, J., Rahman, S. A., Ehtesham, N. Z., Hira, S. & Hasnain, S. E. SARS-CoV-2 variants of concern are emerging in India. Nat Med, 1-3 (2021).
22. Organization, W. H. COVID-19 weekly epidemiological update, edition 43, 8 Jun. 2021. (2021).
23. Bernal, J. L. et al. Effectiveness of COVID-19 vaccines against the B. 1.617. 2 variant. medRxiv (2021).
24 Saito, A. et al. SARS-CoV-2 spike P681R mutation enhances and accelerates viral fusion. bioRxiv (2021).
25. Bugembe, D. L. et al. A SARS-CoV-2 lineage A variant (A. 23.1) with altered spike has emerged and is dominating the current Uganda epidemic. MedRxiv (2021).
26. Guthmiller, J. J. et al. SARS-CoV-2 Infection Severity Is Linked to Superior Humoral Immunity against the Spike. Mbio 12, doi:ARTN e02940-20 10.1128/mBio.02940-20 (2021).
27 Dugan, H. L. et al. Profiling B cell immunodominance after SARS-CoV-2 infection reveals antibody evolution to non-neutralizing viral targets. Immunity (2021).
28. Ju, B. et al. Human neutralizing antibodies elicited by SARS-CoV-2 infection. Nature 584, 115-+, doi: 10.1038/s41586-020-2380-z (2020).
29 Zost, S. J. et al. Potently neutralizing and protective human antibodies against SARS-CoV-2. Nature 584, 443-449, doi: 10.1038/s41586-020-2548-6 (2020).
30. Greaney, A. J. et al. Mutational escape from the polyclonal antibody response to SARS-CoV-2 infection is largely shaped by a single class of antibodies. bioRxiv, doi: 10.1101/2021.03.17.435863 (2021).
31. Starr, T. N. et al. Prospective mapping of viral mutations that escape antibodies used to treat COVID-19. Science 371, 850-+, doi: 10.1126/science.abf9302 (2021).
32. Greaney, A. J. et al. Complete Mapping of Mutations to the SARS-CoV-2 Spike Receptor-Binding Domain that Escape Antibody Recognition. Cell Host Microbe 29, 44-57 e49, doi: 10.1016/j.chom.2020.11.007 (2021).
33 Lan, J. et al. Structure of the SARS-CoV-2 spike receptor-binding domain bound to the ACE2 receptor. Nature 581, 215-220, doi: 10.1038/s41586-020-2180-5 (2020).
34. Starr, T. N. et al. Deep Mutational Scanning of SARS-CoV-2 Receptor Binding Domain Reveals Constraints on Folding and ACE2 Binding. Cell 182, 1295-+, doi: 10.1016/j.cell.2020.08.012 (2020).
35. Weisblum, Y. et al. Escape from neutralizing antibodies by SARS-CoV-2 spike protein variants. Elife 9, doi:ARTN e61312 10.7554/eLife.61312 (2020).
36. Barnes, C. O. et al. SARS-CoV-2 neutralizing antibody structures inform therapeutic strategies. Nature 588, 682-+, doi: 10.1038/s41586-020-2852-1 (2020).
37 Yuan, M. et al. Structural basis of a shared antibody response to SARS-CoV-2. Science 369, 1119-+, doi: 10.1126/science.abd2321 (2020).
38. Yuan, M., Liu, H., Wu, N. C. & Wilson, I. A. Recognition of the SARS-CoV-2 receptor binding domain by neutralizing antibodies. Biochem Biophys Res Commun 538, 192-203, doi: 10.1016/j.bbrc.2020.10.012 (2021).
39 Cherian, S. et al. Convergent evolution of SARS-CoV-2 spike mutations, L452R, E484Q and P681R, in the second wave of COVID-19 in Maharashtra, India. BioRxiv (2021).
40 Thomson, E. C. et al. Circulating SARS-CoV-2 spike N439K variants maintain fitness while evading antibody-mediated immunity. Cell 184, 1171-+, doi: 10.1016/j.cell.2021.01.037 (2021).
41. Deng, X. et al. Transmission, infectivity, and antibody neutralization of an emerging SARS-CoV-2 variant in California carrying a L452R spike protein mutation. medRxiv, doi: 10.1101/2021.03.07.21252647 (2021).
42. Galson, J. D. et al. Deep Sequencing of B Cell Receptor Repertoires From COVID-19 Patients Reveals Strong Convergent Immune Signatures. Front Immunol 11, 605170, doi: 10.3389/fimmu.2020.605170 (2020).
43 Robbiani, D. F. et al. Convergent antibody responses to SARS-CoV-2 in convalescent individuals. Nature 584, 437-+, doi: 10.1038/s41586-020-2456-9 (2020).
44. Schmitz, A. J. et al. A public vaccine-induced human antibody protects against SARS-CoV-2 and emerging variants. bioRxiv, doi: 10.1101/2021.03.24.436864 (2021).
45 Faria, N. R. et al. Genomics and epidemiology of the P.1 SARS-CoV-2 lineage in Manaus, Brazil. Science, doi: 10.1126/science.abh2644 (2021).
46 Amanat, F. et al. SARS-CoV-2 mRNA vaccination induces functionally diverse antibodies to NTD, RBD and S2. Cell (2021).
47 Chi, X. et al. A neutralizing human antibody binds to the N-terminal domain of the Spike protein of SARS-CoV-2. Science 369, 650-655, doi: 10.1126/science.abc6952 (2020).
48. Ng, K. W. et al. Preexisting and de novo humoral immunity to SARS-CoV-2 in humans. Science 370, 1339-1343, doi: 10.1126/science.abe1107 (2020).
49. Shrock, E. et al. Viral epitope profiling of COVID-19 patients reveals cross-reactivity and correlates of severity. Science 370, doi: 10.1126/science.abd4250 (2020).
50. Chan, C. E. Z. et al. The Fc-mediated effector functions of a potent SARS-CoV-2 neutralizing antibody, SC31, isolated from an early convalescent COVID-19 patient, are essential for the optimal therapeutic efficacy of the antibody. PLOS One 16, e0253487, doi: 10.1371/journal.pone.0253487 (2021).
51. Cao, Y. L. et al. Potent Neutralizing Antibodies against SARS-CoV-2 Identified by High-Throughput Single-Cell Sequencing of Convalescent Patients' B Cells. Cell 182, 73-+, doi: 10.1016/j.cell.2020.05.025 (2020).
52. Stuart, T. et al. Comprehensive Integration of Single-Cell Data. Cell 177, 1888-1902 e1821, doi: 10.1016/j.cell.2019.05.031 (2019).
53. Ye, J., Ma, N., Madden, T. L. & Ostell, J. M. IgBLAST: an immunoglobulin variable domain sequence analysis tool. Nucleic Acids Res 41, W34-40, doi: 10.1093/nar/gkt382 (2013).
54. Guthmiller, J. J., Dugan, H. L., Neu, K. E., Lan, L. Y. & Wilson, P. C. An Efficient Method to Generate Monoclonal Antibodies from Human B Cells. Methods Mol Biol 1904, 109-145, doi: 10.1007/978-1-4939-8958-4_5 (2019).

### Example 2: A Broadly Protective Antibody to Emerging SARS-CoV-2 Variants Binds an Epitope More Readily Accessible on Hexaproline Spike Antigen Constructs

The rapid evolution of SARS-CoV-2 Omicron variants has emphasized the need to identify antibodies with broad neutralizing capabilities to inform future monoclonal therapies and vaccination strategies. Herein, the inventors identify S728-1157, a broadly neutralizing antibody (bnAb) targeting the receptor-binding site (RBS) and derived from an individual previously infected with SARS-CoV-2 prior to the spread of variants of concern (VOCs). S728-1157 demonstrates broad cross-neutralization of all dominant variants including D614G, Beta, Delta, Kappa, Mu, and Omicron (BA.1/BA.2/BA.2.75/BA.4/BA.5). Furthermore, it protected hamsters against in vivo challenges with wildtype, Delta, and BA.1 viruses. Structural analysis reveals that this antibody targets a class 1 epitope via multiple hydrophobic and polar interactions with its CDR-H3, in addition to common class 1 motifs in CDR-H1/CDR-H2. Importantly, this epitope is more readily accessible in the open and prefusion state, or in the hexaproline (6P)-stabilized spike constructs, as compared to diproline (2P) constructs. Overall, S728-1157 demonstrates broad therapeutic potential, and may inform target-driven vaccine design against future SARS-CoV-2 variants.

### A. Introduction

Since the start of the pandemic in December 2019, the severe acute respiratory syndrome coronavirus 2 (SARS-CoV-2) virus has led to over 576 million cases of coronavirus disease 2019 (COVID-19) and over six million deaths globally. Although the rapid development and distribution of vaccines and therapeutics has curbed the impact of COVID-19 to an extent, the emergence of circulating variants of concern (VOCs) continues to represent a major threat due to the potential for further immune evasion and enhanced pathogenicity. The D614G variant was the earliest variant to emerge and became universally prevalent thereafter. In comparison to wildtype (WT), the D614G variant exhibited increased transmissibility rather than increased pathogenicity and was therefore unlikely to reduce efficacy of vaccines in clinical trials (1). Between the emergence D614G and October 2021, four additional significant VOC evolved worldwide, including Alpha, Beta, Gamma, and Delta. Among these variants, Delta became a serious global threat as a result of its transmissibility, increased disease severity, and partial immune evasion as shown by the reduced ability of polyclonal serum and monoclonal antibodies (mAbs) to neutralize this strain (2-6). Shortly afterwards, in November 2021, the Omicron variant was identified and announced as a novel VOC. This variant possessed the largest number of mutations to date and appeared to spread more rapidly than previous strains (7,8). Currently, there are five major subvariant lineages of Omicron (BA.1, BA.2, BA.3, BA.4 and BA.5) leading to new COVID-19 cases, with BA.5 becoming dominant over BA.2 and accounting for most new cases in the United States at the time of writing. The Omicron variants can escape recognition by COVID-19 vaccine-associated immunity to varying extents, thereby significantly reducing the neutralizing potency of serum antibodies from convalescent and fully mRNA-vaccinated individuals (9). Similarly, Omicron variants were able to escape binding of several Emergency Use-Authorization (EUA) therapeutic mAbs even though these had been previously shown to be effective against earlier VOCs (10,11). Due to the lowered neutralization against Omicron and the continued threat of future VOCs, there is an urgent need to identify broad and potent neutralizing antibodies that can protect against diverse evolving SARS-CoV-2 lineages.

In this study, the inventors identify a potent RBD-reactive monoclonal antibody from the peripheral blood of SARS-CoV-2 convalescent individual that effectively neutralize Alpha, Beta, Kappa, Delta, Mu, and Omicron variants (BA.1, BA.2, BA.2.75, BA.4 and BA.5). This mAb, S728-1157, entirely abrogated BA.1 Omicron replication in vivo and significantly reduced viral loads during wildtype and Delta infection. In terms of specificity, S728-1157 bound the receptor binding site (RBS) that is fully exposed when the RBD on the spike is in the up conformation. S728-1157 binds using motifs found in the CDR-H1 and CDR-H2 domains that are common to IGHV3-53/3-66 class 1 antibodies but also via extensive unique contacts with CDR-H3 to circumvent mutations in the variant virus spikes. This suggests that the rational design of future vaccine boosts covering Omicron variants should be modified to present stabilized spike in the up configuration to optimally induce class 1 mAbs that have similar CDR-H3 features.

### B. Results

1. Isolation of RBD-Reactive mAbs that Exhibit Diverse Patterns of Neutralization and Potency

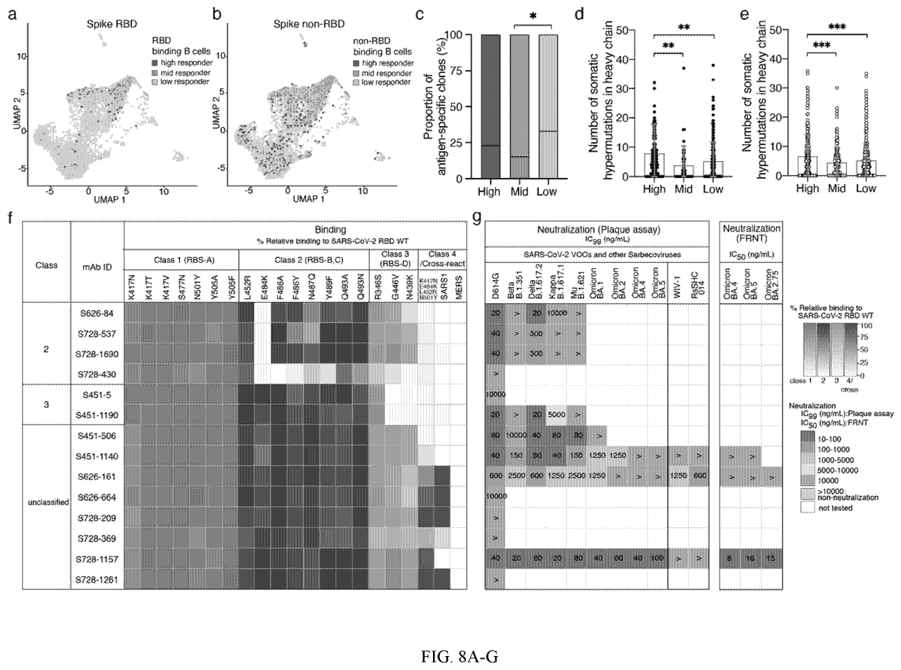

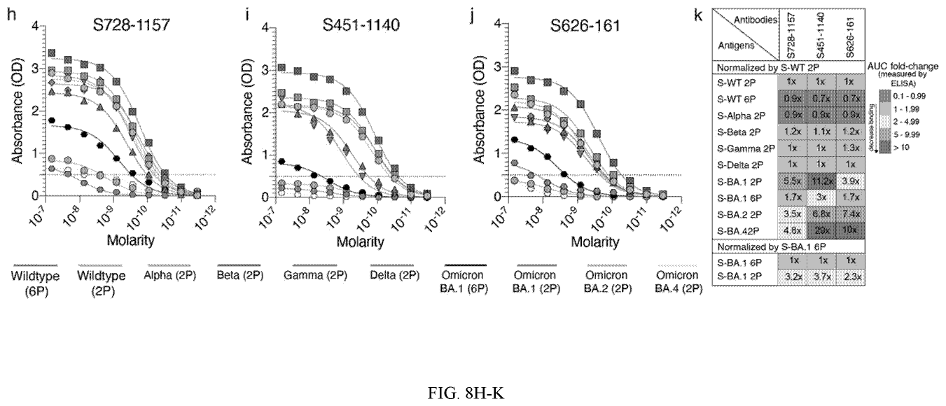

Before the spread of the Omicron variant, the inventors previously characterized 43 mAbs targeting distinct epitopes on the spike protein, including the N-terminal domain (NTD), RBD, and subunit 2 (S2), although none were able to neutralize all existing SARS-CoV-2 variants at that time (12). In the current study, an additional panel of RBD-reactive mAbs were expressed from three high-responder subjects who mounted robust anti-spike IgG responses, as defined previously (Table S1 and Table S2) (13). Although the proportion of spike RBD-binding B cells was similar in high-responders as compared to mid- and low-responders (FIG. 8a-c), heavy chain somatic hypermutation rates were significantly greater in the high-responder group (FIG. 8d), suggesting that these subjects may have the highest potential to generate potent cross-reactive mAbs (13). These antibodies were assayed for binding to key RBD mutants to identify their epitope classifications (Table S3) (14). Among 14 RBD-reactive mAbs, the inventors identified four class 2 mAbs, two class 3 mAbs, and eight unclassified mAbs that showed little to no reduction of binding against any key RBD mutants tested (FIG. 8f). Class 2 and 3 RBD mAbs did not recognize a multivariant RBD mutant containing K417N/E484K/L452R/N501Y substitutions, an artificially designed RBD to include the key mutations for virus escape (14,15), nor cross-reactivity to the RBD of SARS-CoV-1 and Middle Eastern respiratory syndrome (MERS)-CoV (FIG. 8f). Functionally, class 2 and 3 RBD mAbs potently neutralized D614G and Delta but neutralizing activity was limited against Beta, Kappa and Mu (FIG. 8g). No class 2 or 3 antibodies assayed could neutralize any tested Omicron variant.

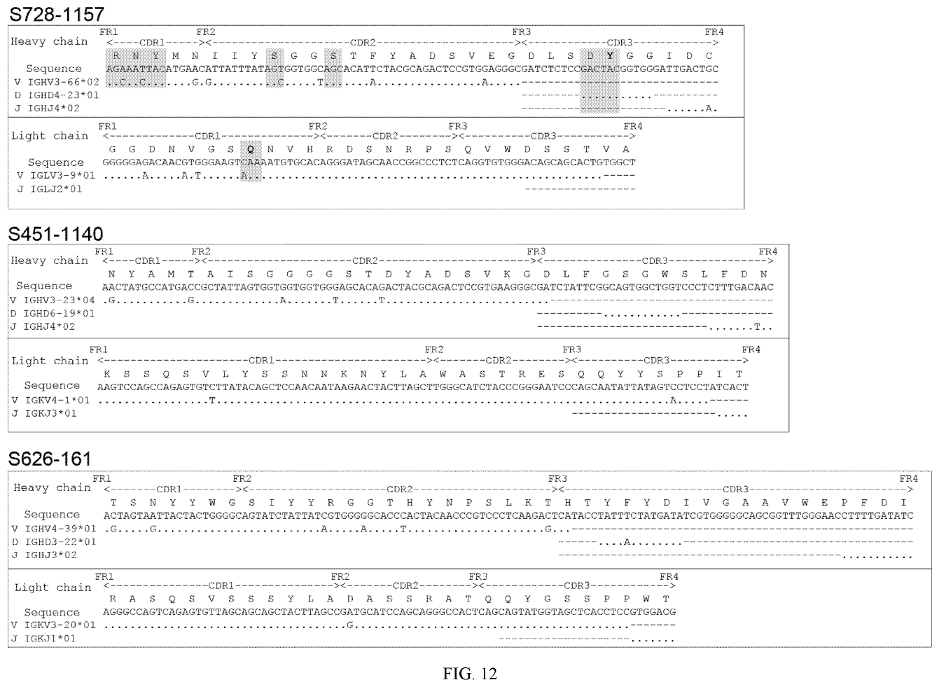

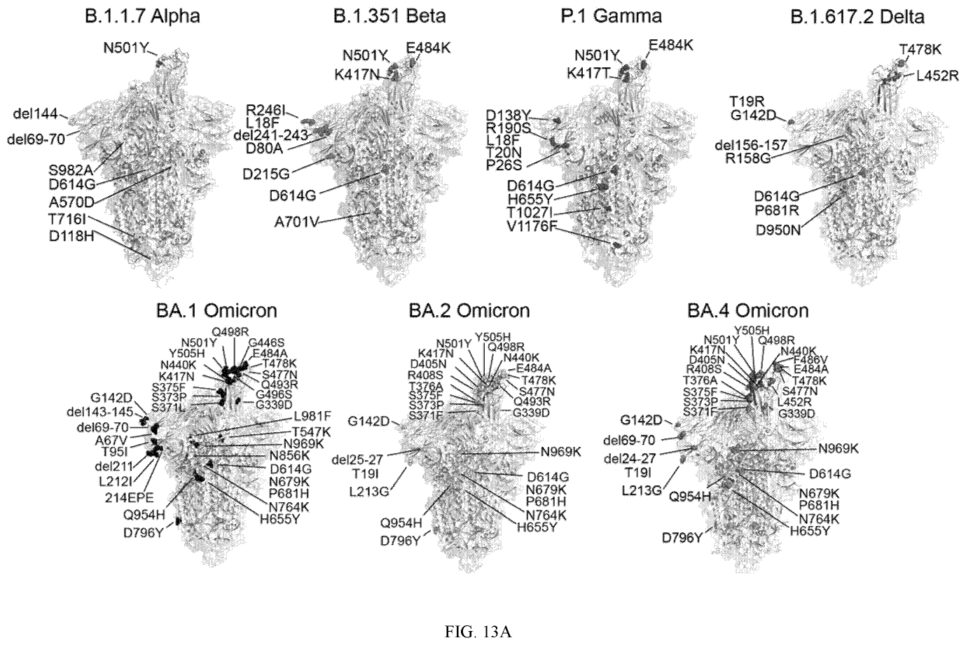

In contrast, the majority of unclassified mAbs bound to the RBD multivariant and cross-reacted to the SARS-CoV-1 RBD (FIG. 8f). Among these, the inventors went on to identify three bnAbs, S451-1140, S626-161 and S728-1157, that showed high neutralization potency against D614G and could cross-neutralize Beta, Delta, Kappa, Mu and BA.1 with 99% inhibitory concentration (IC99) in the range of 20-2500 ng/ml (FIG. 8g). Given the broad neutralization potency of these three mAbs, in addition of plaque assay platform, the inventors also performed the neutralization activity against authentic BA.4, BA.5 and BA.2.75 viruses using focus reduction neutralization test (FRNT) (FIG. 8g). Of these, S728-1157 displayed high neutralizing activities against the panel of Omicron variants including BA.1, BA.2, BA.4 and BA.5, with IC99 below 100 ng/ml as measured by plaque assay. A similar scenario was observed using FRNT, S728-1157 maintains its extremely high neutralization activity against BA.2.75, BA.4 and BA.5 with 50% inhibitory concentration (IC50) in the range of 8-16 ng/ml (FIG. 8g). S451-1140 neutralized BA.1 and BA.2 potently, but not BA.4 and BA.5 as observed in both neutralization assay platforms. On the other hand, S626-161 did not demonstrate neutralizing activity against Omicron variants beyond the BA.1 variant (FIG. 8g). Although S626-161 had a lower neutralization potency against VOC than the other two, it was the only mAb which showed cross-reactivity to SARS-CoV-1 RBD and was able to neutralize bat coronaviruses WIV-1 and RsSHC014 (FIG. 8f-g). These data suggest that S626-161 recognizes a conserved epitope that is shared between these sarbecovirus lineages, but is absent in BA.2. Additionally, compared to S728-1157 and S451-1140, S626-161 has a longer CDR-H3 which could provide an enhanced capability to recognize a highly conserved patch of residues shared across sarbecoviruses as described in a previous study (16) (FIG. 12). When comparing immunoglobulin heavy (IGHV) and light chain (IGLV or IGKV) variable genes of these three bnAbs with the available SARS-CoV-2 neutralizing mAbs database (12, 17-25), the inventors found that heavy chain variable genes utilized by S728-1157 (IGHV3-66), S451-1140 (IGHV3-23) and S626-161 (IGHV4-39) have been previously reported to encode several potently neutralizing SARS-CoV-2 antibodies targeting the RBD (18, 19, 26, 27). However, our mAbs had unique heavy and light chain variable gene pairings that have not been reported in the database (Table S2), indicating that they are not public clonotypes.

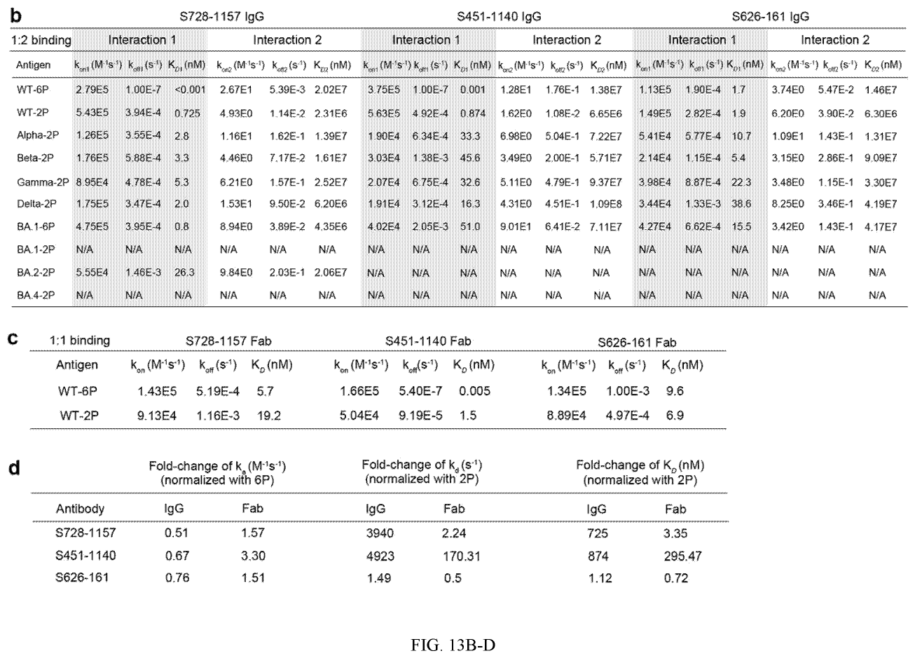

These three bnAbs (S451-1140, S626-161 and S728-1157) were characterized further to determine the binding breadth against SARS-CoV-2 VOCs (FIG. 8h-k). The prefusion-stabilized spike containing two-proline substitutions in the S2 subunit (2P; diproline) has been shown to be a superior immunogen compared to the wildtype spike and is the basis of several current SARS-CoV-2 vaccines, including current mRNA-based vaccines (28,29). More recently, spike protein stabilized with six prolines (6P; hexaproline) was shown to boost expression and be even more stable than the original diproline construct; as a result, it has been proposed for use in improving the next-generation of COVID-19 vaccines (30,31). To determine if there are antigenicity differences between the diproline and hexaproline spike constructs, both immunogens were included in our test panel. As measured by ELISA assay, the inventors found that three bnAbs bound 6P-WT spike antigen to a greater extent compared to WT-2P spike (FIG. 8h-j). All three bnAbs showed comparable binding to the spikes of Alpha, Beta, Gamma and Delta viruses, relative to that of WT-2P (FIG. 8h-j). However, the binding reactivity of these three bnAbs were substantially reduced against a panel of Omicron-family antigens (FIG. 8h-k). S451-1140 binding was sensitive to mutations found in BA.1 and BA.2, resulting in a decrease in binding of more than 3-fold (range of 3- to 11.2-fold) and a 31-fold decrease in neutralization against these variants compared with WT-2P antigen and D614G virus, respectively (FIG. 8g, i, k). The sarbecovirus-cross neutralizing mAb, S626-161 also showed 1.7 to 3.9-fold reduced binding to spike BA.1 antigens and thereby resulted in a 2-fold reduction in neutralization activity against BA.1 (FIG. 8g, j, k). For the most potent bnAb, S728-1157, binding to Omicron antigens was substantially reduced by greater than 1.7-fold (range of 1.7- to 5.5-fold) compared with WT-2P spike but was unaffected in neutralizing activity (FIG. 8g, h, k). Notably, all three bnAbs showed over 3-fold increased binding to spike BA.1-6P compared with the BA.1-2P version, suggesting a better accessibility of bnAbs to the hexaproline spike BA.1 construct. In addition to ELISA, biolayer interferometry (BLI) was used to quantify the binding rate and equilibrium constants (kon, koff, and K_{D}) of these three bnAbs to a panel of spike antigens (FIG. 13b-d). The recognition kon rates of Fabs were 1.5 to 3.3-fold faster to hexaproline spikes, showing that the antibodies bound to the 6P construct more rapidly than to 2P. This is expected if the epitopes are more exposed on the RBD in the open state on the hexaproline spike (FIG. 13c). Except for S626-161, off-rate of the Fabs were also longer such that the overall K_{D} showed that S728-1157 and S451-1140 bound to the hexaproline spike with substantially greater affinity (FIG. 13c-d). The increased off rates further suggest partial occlusion of the binding site on diproline spike. The improved binding to hexaproline spike was even more notable for whole dimeric IgG by the 1:2 interaction model and for all three bnAbs, consistent with exposure of multiple epitopes with 6P stabilization allowing improved avidity (FIG. 13b-d). Taken together, these results suggest that the epitopes targeted may be comparatively more accessible on the 6P-stabilized spike with the RBD in the open state. Structural analyses were next performed to verify this conjecture.

### 2. Structural Analysis of Broadly Neutralizing Monoclonal Antibodies

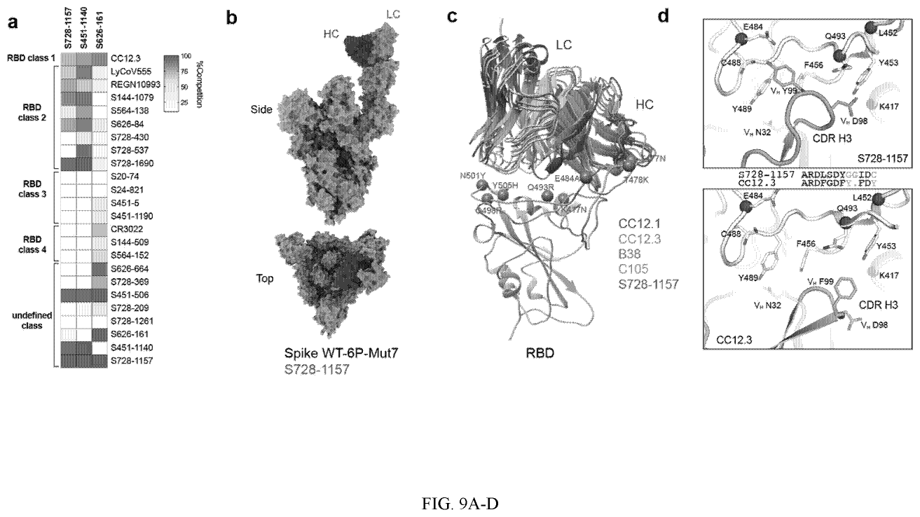

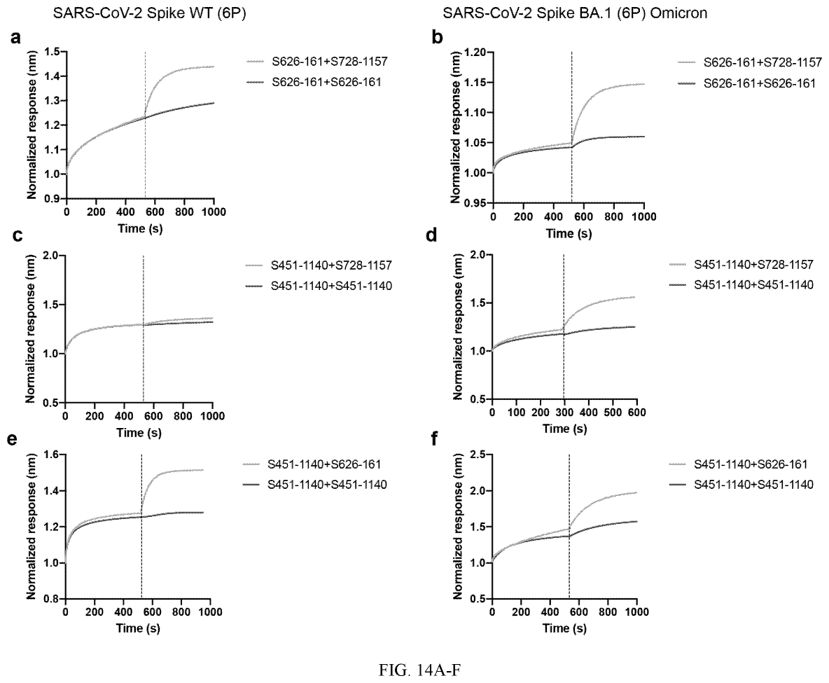

As a first approximation of epitopes bound, an ELISA competition assay was used to determine whether the three broadly-neutralizing mAbs shared any overlap with our current panel of mAbs and a collection of mAbs with known epitope specificities from previous studies (12, 32, 33), plus two other mAbs currently in clinical use (LY-CoV555 (Eli Lilly) (34) and REGN10933 (Regeneron) (35)). The binding sites of S451-1140 and S728-1157 partially overlapped with CC12.3 (33,36), a class 1 neutralizing antibody, and most class 2 antibodies, including LY-CoV555 and REGN10933, but not with class 3 and class 4 antibodies (FIG. 9a). S626-161 shared a notable overlap in binding region with class 1 CC12.3, several class 4 antibodies including CR3022, and other unclassified antibodies, while having some partial overlap with several class 2 and one class 3 antibodies (FIG. 9a). Analogously, competition BLI assay revealed that S451-1140 and S728-1157 strongly competed with one another for binding to spike WT-6P, whereas S626-161 did not (FIG. 14). Overall, these data suggest S451-1140 and S728-1157 recognize similar epitopes that are distinct from S626-161.

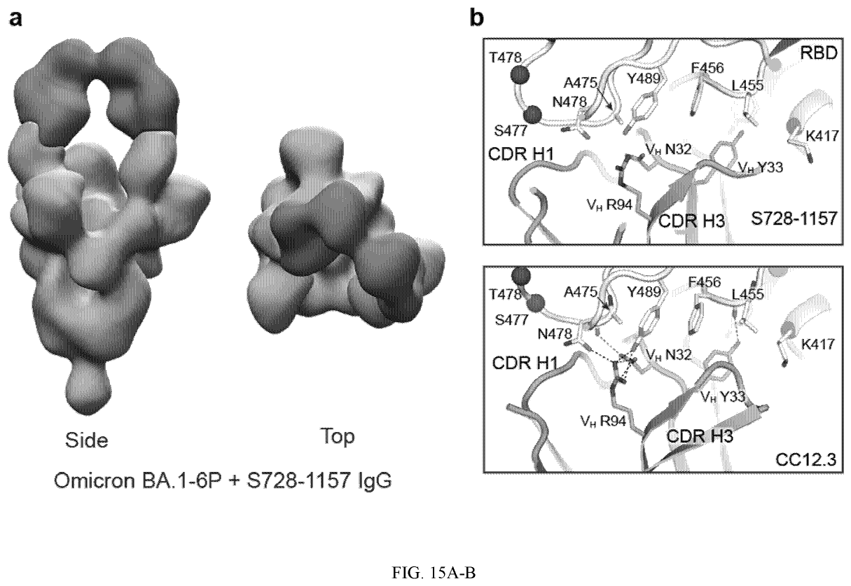

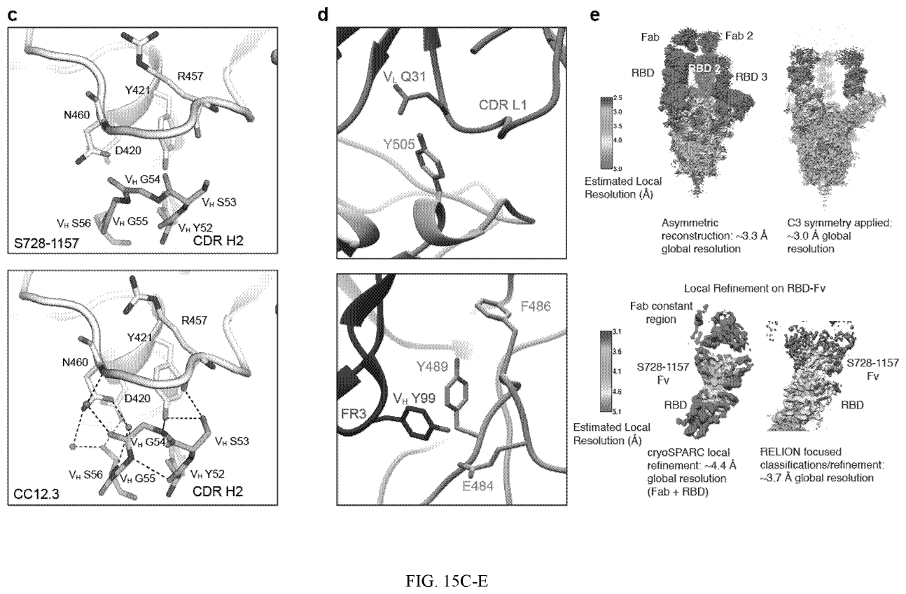

S728-1157 was encoded by IGHV3-66 and possessed a short complementarity determining region 3 (CDR-H3). Notably, mAbs that bind the receptor binding site (RBS) in binding mode 1 (i.e. RBS-A or class 1 site), typified by CC12.1, CC12.3, B38, and C105 (15, 25, 27, 36-38), tend to use IGHV3-53/3-66 and are sensitive to VOC mutations (39). However, the CDR-H3 region of S728-1157 is highly distinct from other antibodies of this class, potentially accounting for its broader activity. To understand the structural basis of broad neutralization by S728-1157 at this epitope, the inventors resolved a cryo-electron microscopy (cryo-EM) structure (FIG. 9b) of IgG S728-1157 in complex with spike WT-6P-Mut7, a version of spike WT-6P possessing interprotomer disulfide bond at C705 and C883, at ˜3.3 Å global resolution (FIG. 15e).

Using symmetry expansion, focused classification, and refinement methods, the inventors achieved local resolution at the RBD-Fv interface to ˜4 Å (FIG. 15e and Table S6). A crystal structure of S728-1157 Fab was determined at 3.1 Å resolution and used to build the atomic model at the RBD-Fv interface. Our structures confirm that S728-1157 binds the RBS-A (or class 1) epitope in the RBD-up conformation (FIG. 9b and FIG. 15e), similar to other IGHV3-53/3-66 antibodies (FIG. 9c). Steric blockage of the angiotensin converting enzyme 2 (ACE2) binding site by S728-1157 explains its high neutralization potency against SARS-CoV-2. The ₃₂NY₃₃ motif and ₅₃SGGS₅₆ motif (36) in S728-1157 CDR-H1 and-H2 interact with the RBD in almost the same way as CC12.3 (FIG. 15b-c). However, V_{H 98}DY₉₉ in S728-1157 CDR-H3 forms more extensive interactions including both hydrophobic and polar interactions with the RBD, compared to V_{H 98}DF ₉₉ in CC12.3 (FIG. 9d and Table S5). The diglycine V_{H 100}GG₁₀₁ in S728-1157 CDR-H3 may also facilitate more extensive binding compared to VH Y₁₀₀ in CC12.3 likely due to the flexibility in the glycine residues that lead to a different conformation of the tip of the CDR-H3 loop and a relative shift of residues at 98DY 99.

Although the Omicron VOCs have extensive mutations in the RBD (FIG. 9c and FIG. 13a), most of these residues do not make interactions with or are dispensable for binding to S728-1157, as binding is still observed (FIG. 15a). From our spike WT-6P-Mut7+Fab S728-1157 model, Y505 to V_{L} Q31, and E484 to V_{H} Y99 are predicted to make hydrogen bonds (FIG. 15d and Table S5), which have the potential to be disrupted by Omicron mutations Y505H and E484A. However, a Y505H mutation would still allow for a hydrogen bond with VL Q31 and an E484A mutation would add another hydrophobic side chain near hydrophobic residues VL Y99, F456, and Y489. These contacts may explain the mechanism which enabled S728-1157 to retain neutralizing activity (FIG. 8g), albeit reduced binding reactivity against spike BA.1 antigen, which is in turn possibly due to the function of Omicron mutations in altering the conformational landscape of the spike protein (40). Notably, while the variable genes were well-mutated, all but one of the contact residues between the CDR-H3 of S728-1157 and the VOC were predicted to be germline encoded and not introduced by somatic mutations, likely limiting the number of existing memory B cells of this class that could be further adapted by somatic mutation to protect against VOC strains (FIG. 12, Table S5). Overall, our structural studies revealed the basis of broad neutralization of S728-1157 that can accommodate most mutations in the SARS-CoV-2 VOCs.

### 3. S728-1157 Reduces Replication of SARS-CoV-2 Delta and Omicron Variants in Syrian Hamsters

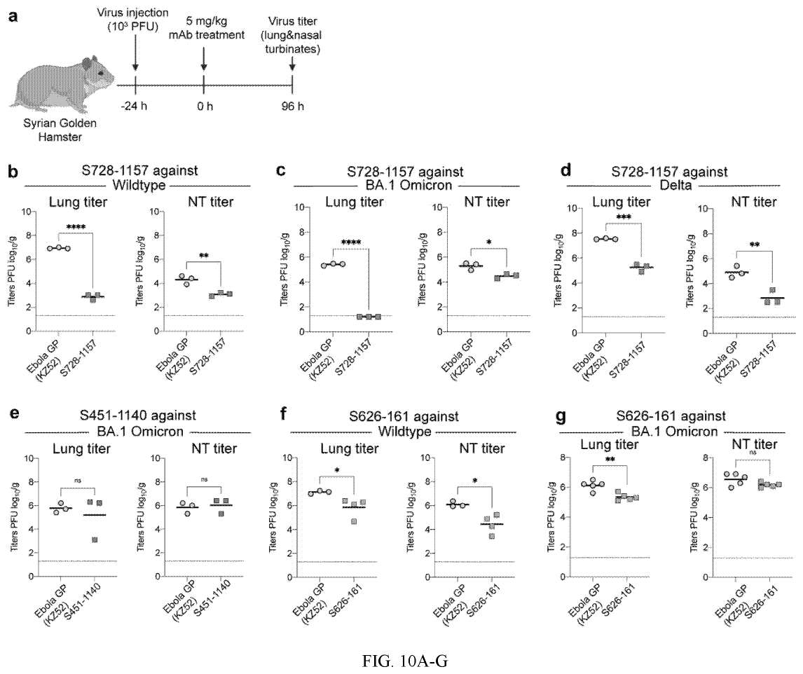

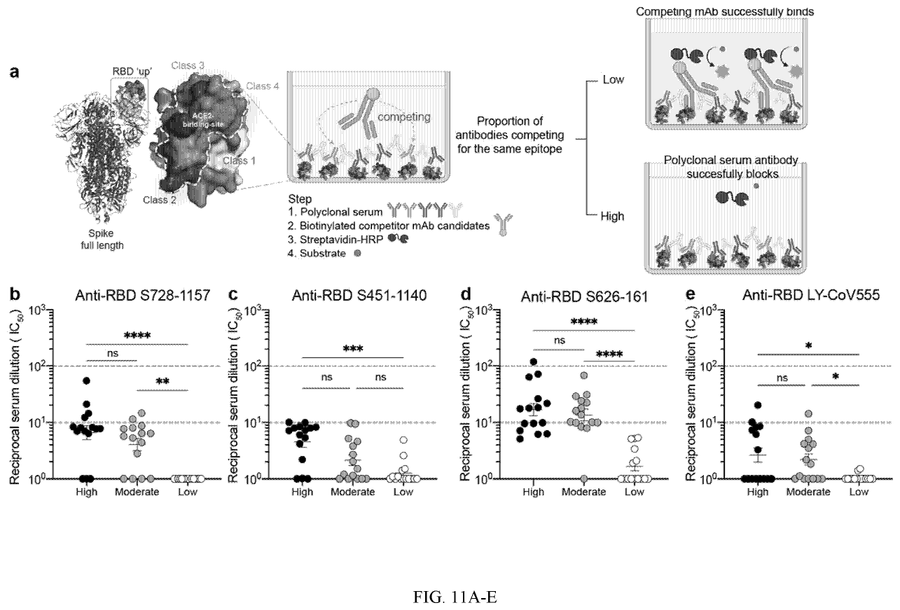

To evaluate the protective efficacy of our broadly neutralizing mAbs, the inventors utilized a golden Syrian hamster infection model that has been widely used for SARS-CoV-2 infection. Hamsters received 5 mg/kg of individual mAbs or an irrelevant antigen (ebolavirus glycoprotein)-specific isotype control via intraperitoneal injection one day post-infection with SARS-CoV-2 viruses. Lung and nasal tissues were collected at 4 days post-infection (dpi) (FIG. 10a). Therapeutic administration of S728-1157 resulted in reduced titers of wildtype, BA.1 Omicron and Delta variants in both the nasal turbinates and lungs of infected hamsters (FIG. 10b-d). Interestingly, the effect of S728-1157 in the lungs was dramatic, reducing wildtype viral loads by ˜10⁴ PFU, and BA.1 Omicron by ˜10⁵ PFU, with replication of the latter being completely abolished (FIG. 10c). In contrast to in vitro neutralization, S451-1140 did not reduce BA.1 Omicron viral replication in lung and nasal turbinates, indicating the disconnect between in vitro neutralization and in vivo protection for S451-1140 (FIG. 10e). In comparison, S626-161 administration resulted in significant but marginal reductions in lung viral titers following wildtype and BA.1 challenge (FIG. 10f-g). These data underscore that to precisely define broadly protective mAbs, evaluating protection efficacy in parallel with neutralization activity is required. Overall, S728-1157 represents a promising mAb with broad neutralization efficacy against SARS-CoV-2 variants that is capable of dramatically reducing wildtype, Delta and BA.1 replication in vivo.

4. SARS-CoV-2 Infection Rarely Elicits Potent S728-1157-Like Cross-Neutralizing mAbs

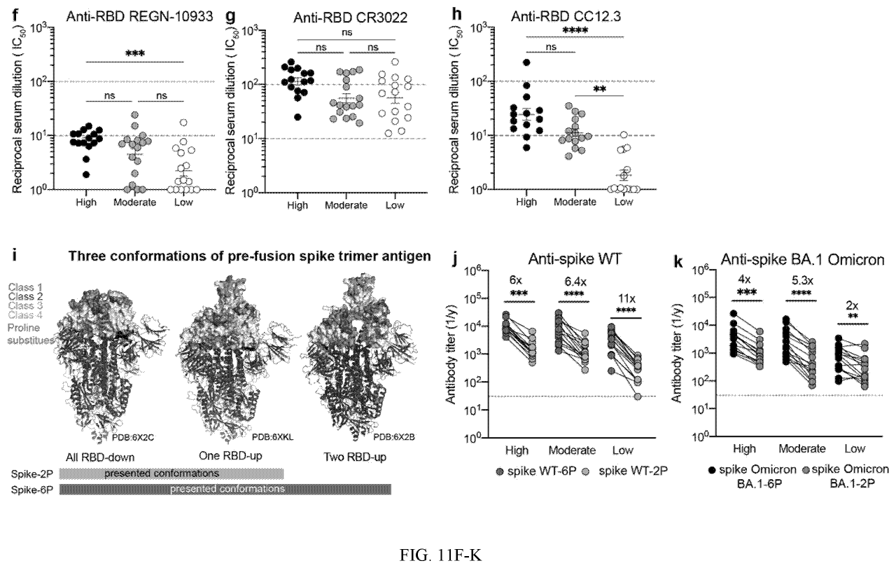

Given the cross-neutralization and prophylactic potential of S728-1157, the inventors sought to evaluate whether S728-1157-like antibodies are commonly induced among polyclonal responses in SARS-CoV-2 patients. To assess this, the inventors performed competition ELISAs using convalescent serum to detect anti-RBD antibody titers that could compete for binding with S728-1157 (FIG. 11a). Subjects were divided into three groups based on their magnitude of antibody responses, as defined previously (12,13). Although high- and moderate-responders had higher titers of S728-1157-competitive serum antibodies compared to low-responders (FIG. 11b), the titers were quite low across all groups suggesting that it is uncommon to acquire high levels of S728-1157-like antibodies in polyclonal serum following wildtype SARS-CoV-2 infection. In addition to S728-1157, the inventors tested the competition of convalescent serum with other mAbs, including S451-1140 and S626-161, LY-CoV555, REGN10933, CR3022, and CC12.3. Similar to S728-1157, the inventors observed relatively low titers of antibodies competing with S451-1140, S626-161, LY-CoV555, REGN10933 and CC12.3 in polyclonal serum from most of the convalescent individuals (FIG. 11c-f, h). Nonetheless, high-responders tended to have significantly higher titers against those neutralizing mAbs than low-responders (FIG. 11b-f, h). In contrast, antibodies targeting the CR3022 epitope site were more pronounced in convalescent individuals, suggesting the enrichment of class 4 RBD antibodies in polyclonal serum (FIG. 11g). Notably, there was no difference in titers of CR3022 across the three responder groups, suggesting that CR3022-site antibodies were largely induced during wildtype SARS-CoV-2 infection. Interestingly, as compared to CC12.3, S728-1157 was detected at 4-fold lower levels in the serum of high-responders. Thus, despite class 1 antibodies being frequently induced by natural infection and vaccination (17, 26, 27, 41-44), our data suggest that S728-1157-like antibodies represent a subset of this class that are comparatively rare.

Lastly, the inventors examined the difference in reactivity to 2P-versus 6P-stabilized spike in our convalescent cohort sera (FIG. 11i-k). The inventors found that all three responder groups mounted anti-spike reactive antibodies against 6P-stabilized spike wildtype to a greater extent than 2P-stabilized spike wildtype, by a factor of 6 to 11-fold (FIG. 11j), indicating that the major antigenic epitopes were better exhibited or stabilized on 6P-stabilized antigen. Using the same samples, high and moderate responders also had lower anti-spike antibodies against BA.1-2P than BA.1-6P, by 4 to 5-fold (FIG. 11k). Of note, low responders had a smaller fold change in binding reactivity against spike BA.1 Omicron-2P and 6P (2-fold reduction) compared to wildtype-2P and 6P spike (11-fold reduction) (FIG. 11j-k), suggesting that serum antibody against BA.1 Omicron-reactive epitopes may be limited in low responder subjects. Overall, these data suggest that there is improved polyclonal binding induced by natural infection to 6P-stabilized spike, both for wildtype and Omicron viruses.

### C. Materials and Methods

### 1. Monoclonal Antibody Isolation

The inventors isolated a panel of RBD-reactive mAbs from peripheral blood mononuclear cells (PBMCs) of convalescent donors who previously had experienced symptomatic infection with SARS-CoV-2 (Table S1). The samples were collected during the first wave of the pandemic in May 2020, before other SARS-CoV-2 variants emerged. All studies were performed with the approval of the University of Chicago institutional review board (IRB20-0523). All participants provided prior written informed consent for the use of blood in research applications. This clinical trial was registered at ClinicalTrials.gov under identifier NCT04340050.

PBMCs were isolated from leukoreduction filters and frozen as described previously (21). B cells were enriched from PBMCs via fluorescence-activated cell sorting (FACS). Cells were stained with CD19, CD3, and antigen probes conjugated oligo-fluorophore; cells of interest were identified as CD3⁻CD19⁺Antigen⁺. All mAbs were generated from oligo-tagged antigen bait-sorted cells identified through single-cell RNA sequencing (RNA-seq), as described previously (12,21).

Antigen-specific B cells were selected to generate mAbs based on antigen-probe intensity analyzed by JMP Pro 15. Antibody heavy and light chain genes were synthesized and cloned into human IgG1 and human kappa or lambda light chain expression vectors by Gibson assembly as previously described (56). The heavy and light chains of the corresponding mAb were transiently co-transfected into HEK293T cells. After transfection for 18 h, the transfected cells were supplemented with Protein-Free Hybridoma Medium Supernatant (PFHM-II, Gibco). The supernatant containing secreted mAb was harvested at day 4 and purified using protein A-agarose beads (Thermo Fisher) as detailed previously (56).

### 2. Recombinant Spike Protein Expression

The recombinant D614G SARS-CoV-2 full-length (FL) spike, WT RBD, single RBD mutants (R346S, K417N, K417T, G446V, L452R, S477N, F486A, F486Y, N487Q, Y489F, Q493A, Q493N, N501Y, Y505A, Y505F), combination RBD mutant (K417N/E484K/L452R/NN501Y), SARS-CoV-1 RBD and MERS-CoV RBD were generated in-house. Briefly, the recombinant antigens were expressed using Expi293F cells. The gene of interest was cloned into mammalian expression vector (in-house modified AbVec) and transfected using ExpiFectamine 293 kit according to the manufacturer's protocol. The supernatant was harvested at day 4 after transfection and incubated with Ni-nitrilotriacetic acid (Ni-NTA) agarose (Qiagen). The purification was carried out using gravity flow column and eluted with imidazole-containing buffer as previously described (57,58). The eluate was buffering-exchanged with PBS using Amicon centrifugal unit (Millipore). The recombinant FL spikes derived from variants B.1.1.7 Alpha, B.1.351 Beta, P.1 Gamma, B.1.617.2 Delta, BA.1, BA.2 and BA.4 Omicron were produced in the Sather Laboratory at Seattle Children's Research Institute. The K417V, N439K, E484K RBDs and recombinant FL spike WT-2P and 6P were produced in Krammer laboratory at the Icahn School of Medicine at Mount Sinai. The SARS-CoV-2-6P-Mut7 and spike BA.1 Omicron-6P were designed and produced as described in a previous study (59). The protein sequences and resources for each antigen are listed in Table S3.

### 3. Enzyme-Linked Immunosorbent Assay (ELISA)

Recombinant SARS-CoV-2 spike/RBD proteins were coated onto high protein-binding microtiter plates (Costar) at 2 μg/ml in phosphate buffered saline (PBS) at 50 μl/well, and kept overnight at 4° C. Plates were washed with PBS containing 0.05% Tween 20 (PBS-T) and blocked with 150 μl of PBS containing 20% fetal bovine serum (FBS) for 1 h at 37° C. Monoclonal antibodies were serially diluted 3-fold starting from 10 μg/ml in PBS and incubated in the wells for 1 h at 37° C. Plates were then washed and incubated with horseradish peroxidase (HRP)-conjugated goat anti-human IgG antibody (Jackson ImmunoResearch, 1:1000) for 1 h at 37° C. After washing, 100 μl of Super AquaBlue ELISA substrate (eBioscience) was added per well. Absorbance was measured at 405 nm on a microplate spectrophotometer (Bio-Rad). The assays were standardized using control antibodies with known binding characteristics in every plate, and the plates were developed until the absorbance of the control reached an optical density (OD) of 3.0. All mAbs were tested in duplicate, and each experiment was performed twice.

### 4. Serum ELISA

High protein-binding microtiter plates were coated with recombinant SARS-CoV-2 spike antigens at 2 μg/ml in PBS overnight at 4° C. Plates were washed with PBS 0.05% Tween and blocked with 200 μl PBS 0.1% Tween+3% skim milk powder for 1 hour at room temperature (RT). Plasma samples were heat-inactivated for 1 hour at 56° C. before perform serology experiment. Plasma were serially diluted 2-fold in PBS 0.1% Tween+1% skim milk powder. Plates were incubated with serum dilutions for 2 hours at RT. The HRP-conjugated goat anti-human Ig secondary antibody diluted at 1:3000 with PBS 0.1% Tween+1% skim milk powder was used to detect binding of antibodies. After 1-hour of incubation, plates were developed with 100 μl SigmaFast OPD solution (Sigma-Aldrich) for 10 minutes. Then, 50 μl 3M HCl was used to stop the development reaction. Absorbance was measured at 490 nm on a microplate spectrophotometer (BioRad). End point titers were extrapolated from sigmoidal 4PL (where X is log concentration) standard curve for each sample. Limit of detection (LOD) is defined as the mean plus 3 S.D. of the O.D. signal recorded using plasma from pre-SARS-CoV-2 subjects. All calculations were performed in GraphPad Prism software (version 9.0).

### 5. Competition ELISA

To determine the target epitope classification of RBD-reactive mAbs, competition ELISAs were performed using other mAbs with known epitope binding characteristics as competitor mAbs. Competitor mAbs were biotinylated using EZ-Link sulfo-NHS-biotin (Thermo Scientific) for 2 h at room temperature (RT). The excess biotin of biotinylated mAbs was removed with 7k molecular weight-cutoff (MWCO) Zeba spin desalting columns (Thermo Scientific). Plates were coated with 2 μg/ml RBD antigen overnight at 4° C. Plates were blocked with PBS-20% FBS for 2 h at RT, and the 2-fold dilution of the mAbs of an undetermined class, or serum, was added, starting at 20 μg/ml of mAbs and a 1:10 dilution of serum. After antibody incubation for 2 h at RT, the biotinylated competitor mAb was added at a concentration twice that of its dissociation constant (K_{D}) and incubated for another 2 h at RT together with the mAb or serum that was previously added. Plates were washed and incubated with 100 μl HRP-conjugated streptavidin (Southern Biotech) at a dilution of 1:1000 for 1 h at 37° C. The plates were developed with the Super AquaBlue ELISA substrate (eBioscience). To normalize the assays, the competitor biotinylated mAb was added in a well without any competing mAbs or serum as a control. Data were recorded when the absorbance of the control well reached and OD of 1.0-1.5. The percent competition between mAbs was then calculated by dividing a sample's observed OD by the OD reached by the positive control, subtracting this value from 1, and multiplying by 100. For serum, ODs were log₁₀-transformed and analyzed by nonlinear regression to determine the 50% inhibition concentration (IC50) values using GraphPad Prism software (version 9.0). The data were transformed to Log 1P and plotted into graph representative of reciprocal serum dilution of the IC50 of serum dilution that can achieve 50% competition with the competitor mAb of interest. All mAbs were tested in duplicate, each experiment was performed two times independently, and values from two independent experiments were averaged.

### 6. Plaque Assays

Plaque assays were performed with SARS-CoV-2 variant viruses on Vero E6/TMPRSS2 cells (Table S4). Cells were cultured to achieve 90% confluency prior to being trypsinized and seeded at a density of 3×10⁴ cells/well in 96-well plates. On the following day, 10² plaque-forming unit (PFU) of SARS-CoV-2 variant was incubated with 2-fold-diluted mAbs for 1 h. The antibody-virus mixture was incubated with Vero E6/TMPRSS2 cells for 3 days at 37° C. Plates were fixed with 20% methanol and then stained with crystal violet solution. The complete inhibitory concentrations (IC99) were calculated using the log (inhibitor) versus normalized response (variable slope), performed in GraphPad Prism (version 9.0). All mAbs were tested in duplicate, and each experiment was performed twice.

### 7. Focus Reduction Neutralization Test (FRNT)

Focus reduction neutralization test (FRNT) were used to determine neutralization activities as an additional platform beside plaque assay. Serial dilutions of serum starting at a final concentration of 1:20 will be mixed with 103 focus-forming units of virus per well and incubated for 1 h at 37° C. A pooled pre-pandemic serum sample is served as a control. The antibody-virus mixture will be inoculated onto Vero E6/TMPRSS2 cells in 96-well plates and incubated for 1 h at 37° C. An equal volume of methylcellulose solution was added to each well. The cells were incubated for 16 h at 37° C. and then fixed with formalin. After the formalin was removed, the cells were immunostained with a mouse monoclonal antibody against SARS-CoV-1/2 nucleoprotein [clone 1C7C7 (Sigma-Aldrich)], followed by a HRP-labeled goat anti-mouse immunoglobulin (SeraCare Life Sciences). The infected cells were stained with TrueBlue Substrate (SeraCare Life Sciences) and then washed with distilled water. After cell drying, the focus numbers were quantified by using an ImmunoSpot S6 Analyzer, ImmunoCapture software, and BioSpot software (Cellular Technology). The IC50 was calculated from the interpolated value from the log (inhibitor) versus normalized response, using variable slope (four parameters) nonlinear regression performed in GraphPad Prism (version 9.0).

### 8. Negative Stain Electron Microscopy

Spike BA.1 Omicron-6P was complexed with a 0.5-fold molar excess of IgG S728-1157 and incubated for 30 mins at room temperature. The complex was diluted to 0.03 mg/ml and deposited on a glow-discharged carbon-coated copper mesh grid. 2% uranyl formate (w/v) was used to stain the sample for 90 seconds. The negative stain dataset was collected on a Thermo Fisher Tecnai T12 Spirit (120 keV, 56,000× magnification, 2.06 apix) paired with a FEI Eagle 4k×4k CCD camera. Leginon (60) was used to automate the data collection and raw micrographs were store in the Appion database (61). Dogpicker (62) picked particles and the dataset was processed in RELION 3.0 (62). UCSF Chimera (63) was used for map segmentation and figure making.

### 9. Cryo-Electron Microscopy and Model Building

SARS-CoV-2-6P-Mut7 was complexed with a 0.5-fold molar excess of IgG S728-1157 and incubated for 30 mins at room temperature. Grids were prepared using a Thermo Fisher Vitrobot Mark IV set to 4° C. and 100% humidity. The complex, at 0.7 mg/ml, was briefly incubated with lauryl maltose neopentyl glycol (final concentration of 0.005 mM; Anatrace), deposited on a glow-discharged Quantifoil 1.2/1.3-400 mesh grid, and blotted for 3 seconds. The grid was loaded into a Thermo Fisher Titan Krios (130,000× magnification, 300 kEV, 1.045-Å pixel size) paired with a Gatan 4k×4k K2 Summit direct electron detector. The Leginon software was used for data collection automation and resulting images were stored in the Appion database. Initial data processing was performed with cryoSPARC v3.2 (64), which included CTF correction using GCTF (65), template picking, and 2D and 3D classification and refinement methods leading to a ˜3.3 Å Cl global reconstruction. The particles from this reconstruction were imported into Relion 3.1 (66), subjected to C3 symmetry expansion, followed by focused 3D classifications without alignments using a mask around the antibody Fab and S-protein RBD regions of a single protomer. Classes with well-resolved density in this region were selected and subjected to additional rounds of focused classification. Refinements were performed with limited angular searches and a mask around the trimeric S-protein and a single Fab. The final set of particles reconstructed to ˜3.7 Å global resolution.

Model building was initiated by rigid body docking of the x-ray structure of the Fab and a published cryo-EM model of the SARS-CoV-2 spike open state (PDB ID: 6VYB) into the cryo-EM map using UCSF Chimera (63). Manual building, mutagenesis and refinement were performed in Coot 0.9.6 (67), followed by relaxed refinement using Rosetta Relax (68). Model manipulation and validation was also done using Phenix 1.20 (69). More complete data collection, processing and model building statistics are summarized in Table S6. Figures were generated using UCSF ChimeraX (70).

### 10. Crystallization and X-Ray Structure Determination

384 conditions of the JCSG Core Suite (Qiagen) were used for crystal screening of S728-1157 Fab crystals on the robotic CrystalMation system (Rigaku) at Scripps Research. Crystallization trials were set-up by the vapor diffusion method in sitting drops containing 0.1 μl of protein complex and 0.1 μl of reservoir solution. Crystals appeared on day 14, were harvested on day 21, pre-equilibrated in cryoprotectant containing 15% ethylene glycol, and then flash cooled and stored in liquid nitrogen until data collection. Diffraction quality crystals were obtained in solution containing 0.2 M di-Ammonium tartrate, 20% (w/v) polyethylene glycol (PEG) 3350. Diffraction data were collected at cryogenic temperature (100 K) on Scripps/Stanford beamline 12-1 at the Stanford Synchrotron Radiation Lightsource (SSRL). The X-ray data were processed with HKL2000 (71). The X-ray structures were solved by molecular replacement (MR) using PHASER (72) with MR models for the Fabs from PDB ID: 7KN4 (73). Iterative model building and refinement were carried out in COOT (74) and PHENIX (75), respectively. (76)

### 11. Animals and Challenge Viruses

To determine whether mAbs in the panel could reduce viral load in vivo, Syrian hamsters (females, 6-8 weeks old) were intraperitoneally administered 5 mg/kg of candidate mAb 1 day after intranasal infection with 103 PFU of SARS-CoV-2 viruses (an early SARS-CoV-2 isolate, Delta or BA.1 Omicron). Control animals were treated with an Ebola-specific mAb (KZ52) of matched isotype. At day 4 post-infection, lung tissues and nasal turbinate were collected to evaluate viral titers by standard plaque assay on Vero E6/TMPRRSS2 cells. The animal study was conducted in accordance with the recommendations for care and use of animals by the Institutional Animal Care and Use Committee at the University of Wisconsin under BSL-3 containment using approved protocols.

### 12. Biolayer Interferometry (BLI)

To determine precise binding affinity, the dissociation constant (K_{D}) of each mAb was performed by biolayer interferometry (BLI) with an Octet K2 instrument (Forte Bio/Sartorious). The trimeric spike SARS-CoV-2 and its variants were biotinylated (EZ-Link Sulfo-NHS-Biotin, ThermoFisher), desalted (Zeba Spike Desalting, ThermoFisher), and loaded at a concentration of 500 nM onto streptavidin (SA) biosensor (Forte Bio/Sartorious) for 300 s, followed by kinetic buffer (1× PBS containing 0.02% Tween-20 and 0.1% bovine serum albumin) for 60 s. The biosensor was then moved to associate with mAbs of interest (142 nM) for 300 s, followed by disassociation with the kinetic buffer for 300 s. On rate, off-rate, and K_{D} were evaluated with a global fit, the average of those values with high R-squared from two independent experiments were presented. Analysis was performed by Octet Data Analysis HT software (Forte Bio/Sartorious) with 1:1 fitting model for Fabs and 1:2 interacting model for IgG.

For competitive assay by BLI, streptavidin (SA) biosensor was pre-equilibrated in 1×PBS for at least 600 s to bind with the biotinylated trimeric spike WT-6P and spike BA.1 Omicron-6P for 300 s. The first mAb was associated on the loaded sensor for 300 s, followed by the second mAb for another 300 s. The final volume for all the solutions was 200 μl/well. All of the assays were performed with kinetic buffer at 30° C. Data were analyzed by Octet Data Analysis HT software (Forte Bio/Sartorious) and plotted using GraphPad Prism.

### 13. Statistics

All statistical analyses were performed using GraphPad Prism software (version 9.0). The numbers of biological repeats for experiments and specific tests for statistical significance used are described in the corresponding figure legends. P values of ≤0.05 were considered significant [*, P≤0.05; **, P≤0.01; ***, P≤0.001; ****, P<0.0001), while P values of >0.05 were considered as non-significant (ns)].

### D. Tables

Table S1: COVID-19 convalescent subjects. Related to FIG. 8 and FIG. 11. The mAbs from high responder subjects, S451, S626, S728 were characterized in this study. Responder group and severity were categorized in a previous study¹³. Serum antibody from each responder group were tested for competition ELISA with broad neutralizing mAbs, other therapeutic mAbs and non-neutralizing mAb.

| | | | | | Symptom | | |
|---|---|---|---|---|---|---|---|
| | | | | Duration of | start to | | |
| Subject | | | SARS-CoV- | symptoms | donation | Responder | Severity |
| ID | Age | Sex | 2 PCR Test | (days) | (days) | Category26 | Category26 |
| 3 | 20 | M | Mar. 16, 2020 | 4 | 33 | Low | Moderate |
| 11 | 66 | M | Mar. 30, 2020 | 16 | 49 | High | Severe |
| | | | | | | | (hospitalized) |
| 17 | 42 | M | Mar. 21, 2020 | 17 | 55 | High | Severe |
| 19 | 55 | F | Mar. 15, 2020 | 14 | 44 | Low | Moderate |
| 20 | 31 | M | Mar. 31, 2020 | 19 | 48 | High | Critical |
| | | | | | | | (hospitalized) |
| 22 | 31 | F | Mar. 23, 2020 | 3 | 31 | Mid | Moderate |
| 24 | 34 | M | Mar. 23, 2020 | 12 | 41 | High | Severe |
| 42 | 30 | M | Mar. 18, 2020 | 11 | 39 | Mid | Moderate |
| 63 | 44 | M | Mar. 30, 2020 | 2 | 33 | Low | Moderate |
| 80 | 33 | M | Mar. 26, 2020 | 12 | 40 | Mid | Moderate |
| 89 | 64 | M | Mar. 19, 2020 | 13 | 43 | High | Mild |
| 108 | 58 | M | Mar. 15, 2020 | 11 | 39 | High | Moderate |
| 109 | 34 | M | Mar. 15, 2020 | 9 | 41 | Low | Moderate |
| 112 | 43 | M | Mar. 20, 2020 | 9 | 40 | Low | Moderate |
| 116 | 65 | F | Mar. 25, 2020 | 18 | 49 | Low | Moderate |
| 130 | 52 | M | Mar. 26, 2020 | 7 | 35 | Mid | Mild |
| 135 | 28 | F | Mar. 24, 2020 | 7 | 36 | Low | Moderate |
| 141 | 66 | M | Mar. 20, 2020 | 19 | 48 | High | Moderate |
| 144 | 56 | M | Mar. 16, 2020 | 23 | 54 | Low | Moderate |
| 156 | 50 | F | Mar. 23, 2020 | 11 | 41 | High | Moderate |
| 166 | 42 | F | Mar. 25, 2020 | 17 | 55 | Low | Moderate |
| 176 | 26 | M | Mar. 22, 2020 | 6 | 35 | Low | Moderate |
| 210 | 47 | M | Apr. 4, 2020 | 7 | 41 | Low | Moderate |
| 218 | 51 | F | Mar. 16, 2020 | 19 | 48 | Mid | Severe |
| 229 | 55 | M | Mar. 11, 2020 | 2 | 42 | Low | Mild |
| 251 | 53 | M | Mar. 18, 2020 | 22 | 51 | Low | Severe |
| 266 | 20 | F | Mar. 25, 2020 | 4 | 32 | Low | Mild |
| 270 | 50 | M | Mar. 18, 2020 | 9 | 39 | Mid | Moderate |
| 272 | 42 | M | Mar. 18, 2020 | 14 | 43 | Mid | Moderate |
| 277 | 65 | M | Mar. 18, 2020 | 13 | 45 | High | Moderate |
| 278 | 52 | F | Mar. 12, 2020 | 12 | 47 | Mid | Moderate |
| 293 | 72 | M | Mar. 8, 2020 | 17 | 63 | High | Severe |
| | | | | | | | (hospitalized) |
| 305 | 43 | F | Apr. 17, 2020 | 4 | 47 | Low | Moderate |
| 319 | 76 | M | Mar. 27, 2020 | 4 | 36 | High | Mild |
| 332 | 32 | M | Mar. 21, 2020 | 6 | 35 | Mid | Moderate |
| 346 | 30 | M | Mar. 16, 2020 | 11 | 39 | Mid | Moderate |
| 355 | 45 | F | Mar. 14, 2020 | 14 | 44 | Low | Moderate |
| 373 | 48 | M | Mar. 16, 2020 | 7 | 39 | High | Moderate |
| 377 | 44 | M | Mar. 14, 2020 | 9 | 41 | High | Moderate |
| 385 | 33 | M | Mar. 11, 2020 | 7 | 47 | Mid | Moderate |
| 407 | 34 | M | Apr. 1, 2020 | 11 | 43 | Mid | Moderate |
| 433 | 33 | M | Mar. 20, 2020 | 6 | 35 | Low | Moderate |
| 447 | 42 | M | Apr. 1, 2020 | 21 | 61 | High | Severe |
| 451 | 46 | M | Apr. 4, 2020 | 11 | 49 | High | Severe |
| | | | | | | | (hospitalized) |
| 537 | 36 | M | Mar. 23, 2020 | 14 | 59 | Mid | Moderate |
| 564 | 24 | F | Mar. 19, 2020 | 32 | 60 | Low | Severe |
| 573 | 25 | M | Mar. 20, 2020 | 17 | 56 | High | Severe |
| | | | | | | | (hospitalized) |
| 626 | 44 | M | Mar. 31, 2020 | 19 | 56 | High | Moderate |
| 728 | 62 | F | Mar. 15, 2020 | 53 | 130 | High | Severe |

Table S2: Characteristics of SARS-CoV-2 RBD-reactive mAbs. Related to FIG. 8. Cross-neutralizing mAbs against D614G and B.1.351 Beta, B.1., 617.2 Delta, B.1.617.1 Kappa, B. 1.621 Mu, BA.1 Omicron are bolded.

| | Epitope | | | # VH | #VL | CDR-H3 | CDR-L3 |
|---|---|---|---|---|---|---|---|
| mAb ID | specificity | VH gene | VL gene | SHM | SHM | length | length |
| S451-5 | RBD | IGHV2-70*01 | IGLV1-44*01 | 4 | 1 | 12 | 11 |
| | Class 2 | | | | | | |
| S451-506 | RBD | IGHV3-53*02 | IGKV1-9*01 | 9 | 4 | 12 | 10 |
| | Class 3 | | | | | | |
| S451-1140 | RBD | IGHV3-23*04 | IGKV4-1*01 | 8 | 7 | 12 | 9 |
| | Unclassified | | | | | | |
| S451-1190 | RBD | IGHV2-5*02 | IGLV2-14*01 | 8 | 8 | 9 | 11 |
| | Class 3 | | | | | | |
| S626-84 | RBD | IGHV1-2*02 | IGLV2-23*02 | 7 | 9 | 16 | 10 |
| | Class 2 | | | | | | |
| S626-161 | RBD | IGHV4-39*01 | IGKV3-20*01 | 8 | 2 | 18 | 10 |
| | Unclassified | | | | | | |
| S626-664 | RBD | IGHV4-39*01 | IGLV1-51*02 | 8 | 5 | 19 | 10 |
| | Unclassified | | | | | | |
| S728-209 | RBD | IGHV2-5*04 | IGKV1-12*01 | 14 | 12 | 12 | 9 |
| | Unclassified | | | | | | |
| S728-369 | RBD | IGHV4-31*03 | IGKV1-5*03 | 18 | 13 | 23 | 8 |
| | Unclassified | | | | | | |
| S728-430 | RBD | IGHV3-53*01 | IGKV1-33*01 | 1 | 1 | 12 | 10 |
| | Class 2 | | | | | | |
| S728-537 | RBD | IGHV1-2*02 | IGKV1-12*01 | 15 | 9 | 17 | 9 |
| | Class 2 | | | | | | |
| S728-1157 | RBD | IGHV3-66*02 | IGLV3-9*01 | 20 | 9 | 10 | 9 |
| | Unclassified | | | | | | |
| S728-1261 | RBD | IGHV4-4*02 | IGKV3-20*01 | 8 | 12 | 13 | 10 |
| | Unclassified | | | | | | |
| S728-1690 | RBD | IGHV1-69*04 | IGKV3-20*01 | 19 | 8 | 15 | 9 |
| | Class 2 | | | | | | |

Table S3: Antigen information and resource. Proline substitutions are indicated as italic. Related to FIG. 8 and FIG. 13.

| | | | | | |
|---|---|---|---|---|---|
| Antigen | S1 NTD | RBD | S1 CTD | S2 | Source |

Spike FL, trimer

| | | | | | |
|---|---|---|---|---|---|
| Wildtype(WT)-2P | — | — | — | K986P, | Krammer lab |
| | | | | V987P | |
| Wildtype(WT)-6P | — | — | — | F817P, | Krammer lab |
| | | | | A829P, | |
| | | | | A899P, | |
| | | | | A942P, | |
| | | | | K986P, | |
| | | | | V987P | |
| B.1.1.7 Alpha-2P | del69-70, | N501Y | A570D, | T716I, | Sather lab |
| | del144 | | D614G, | S982A, | |
| | | | P681H | K986P, | |
| | | | | V987P, | |
| | | | | D1118H | |
| B.1.351 Beta-2P | L18F, | K417N, | D614G | A701V, | Sather lab |
| | D80A, D215G, | E484K, | | K986P, | |
| | del241-243, | N501Y | | V987P | |
| | R246I | | | | |
| P.1 Gamma-2P | L18F, | K417T, | D614G, | K986P, | Sather lab |
| | T20N, | E484K, | H655Y | V987P, | |
| | P26S, D138Y, | N501Y | | T1027I, | |
| | R190S | | | V1176F | |
| B.1.617.2 Delta-2P | T19R, G142D, | L452R, | D614G, | D950N, | Sather lab |
| | del156-157, | T478K, | P681R | K986P, | |
| | R158G | | | V987P | |
| BA.1 Omicron-2P | A67V, | G339D, | T547K, | N764K, | Sather lab |
| | H69del, | S371L, | D614G, | D796Y, | |
| | V70del, | S373P, | H655Y, | N856K, | |
| | T95I, | S375F, | N679K, | Q954H, | |
| | G142D, | K417N, | P681H | N969K, | |
| | V143del, | N440K, | | L981F, | |
| | Y144del, | G446S, | | K986P, | |
| | Y145del, | S477N, | | V987P | |
| | N211del, L212I, | T478K, | | | |
| | insert214EPE | E484A, | | | |
| | | Q493R, | | | |
| | | G496S, | | | |
| | | Q498R, | | | |
| | | N501Y, | | | |
| | | Y505H | | | |
| BA.1 Omicron-6P | A67V, | G339D, | T547K, | V705C, | Ward lab |
| | H69del, | S371L, | D614G, | N764K, | |
| | V70del, | S373P, | H655Y, | D796Y, | |
| | T95I, | S375F, | N679K, | F817P, | |
| | G142D, | K417N, | P681H | A829P, | |
| | V143del, | N440K, | | N856K, | |
| | Y144del, | G446S, | | T883C, | |
| | Y145del, | S477N, | | A899P, | |
| | N211del, | T478K, | | A942P, | |
| | insert214EPE | E484A, | | Q954H, | |
| | | Q493R, | | N969K, | |
| | | G496S, | | L981F, | |
| | | Q498R, | | K986P, | |
| | | N501Y, | | V987P | |
| | | Y505H | | | |
| BA.2 | T19I, | G339D, | D614G, | N764K, | Sather lab |
| Omicron-2P | L24del, | S371F, | H655Y, | D796Y, | |
| | P25del, | S373P, | N679K, | Q954H, | |
| | P26del, | S375F, | P681H, | N969K, | |
| | A27S, | T376A, | | K986P, | |
| | G142D, | D405N, | | V987P | |
| | V213G, | R408S, | | | |
| | | K417N, | | | |
| | | N440K, | | | |
| | | S477N, | | | |
| | | T478K, | | | |
| | | E484A, | | | |
| | | Q493R, | | | |
| | | Q498R, | | | |
| | | N501Y, | | | |
| | | Y505H | | | |
| BA.4 | T19I, | G339D, | D614G, | N764K, | Sather lab |
| Omicron-2P | L24del, | S371F, | H655Y, | D796Y, | |
| | P25del, | S373P, | N679K, | Q954H, | |
| | P26del, | S375F, | P681H, | N969K, | |
| | A27S, | T376A, | | K986P, | |
| | H69del, | D405N, | | V987P | |
| | V70del, | R408S, | | | |
| | G142D, | K417N, | | | |
| | V213G, | N440K, | | | |
| | | L452R, | | | |
| | | S477N, | | | |
| | | T478K, | | | |
| | | E484A, | | | |
| | | F486V, | | | |
| | | Q498R, | | | |
| | | N501Y, | | | |
| | | Y505H, | | | |

RBD

| | | | | | |
|---|---|---|---|---|---|
| WT | — | — | — | — | In-house |
| R346S | — | R346S | — | — | In-house |
| K417N | — | K417N | — | — | In-house |
| K417V | — | K417V | — | — | Krammer lab |
| K417T | — | K417T | — | — | In-house |
| G446V | — | G446V | — | — | In-house |
| N439K | — | N439K | — | — | Krammer lab |
| L452R | — | L452R | — | — | In house |
| S477N | — | S477N | — | — | In-house |
| E484K | — | E484K | — | — | Krammer lab |
| F486A | — | F486A | — | — | In-house |
| F486Y | — | F486Y | — | — | In-house |
| N487Q | — | N487Q | — | — | In-house |
| Y489F | — | Y489F | — | — | In-house |
| Q493A | — | Q493A | — | — | In-house |
| Q493N | — | Q493N | — | — | In-house |
| N501Y | — | N501Y | — | — | In-house |
| Y505A | — | Y505A | — | — | In-house |
| Y505F | — | Y505F | — | — | In-house |
| K417N/E484K/ | — | K417N/ | — | — | In-house |
| L452R/N501Y | | E484K/ | | | |
| | | L452R/ | | | |
| | | N501Y | | | |

| | |
|---|---|
| SARS-CoV-1 RBD WT | In-house |
| MERS-CoV RBD WT | In-house |

Table S4: SARS-CoV-2 virus information and resource. Related to FIGS. 8 and 10.

| Virus | S1 NTD | RBD | S1 CTD | S2 | Source |
|---|---|---|---|---|---|
| D614G | — | — | D614G | — | 2019-nCoV/USA- |
| | | | | | WA1/2020 D614G |
| B.1.351 | L18F, | K417N, | D614G | A701V | hCoV-19/USA/MD- |
| Beta | D80A, | E484K, | | | HP01542/2021 |
| | D215G, | N501Y | | | |
| | L241del, | | | | |
| | L242del, | | | | |
| | A243del | | | | |
| P.1 | L18F, | K417T, | D614G, | T1027I, | hCoV-19/Japan/TY7- |
| Gamma | T20N, | E484K, | H655Y | V1176F | 501/2021 from BEI |
| | P26S, | N501Y | | | |
| | D138Y, | | | | |
| | G181V, | | | | |
| | R190S | | | | |
| B.1.621 | in3T, | R346K, | D614G, | D950N | hCoV-19/USA/WI-UW- |
| Mu | T95I, | E484K, | P681H | | 4340/2021 |
| | Y144S, | N501Y | | | |
| | Y145N, | | | | |
| B.1.617.1 | G142D, | L452R, | D614G, | Q1071H, | hCoV-19/USA/CA- |
| Kappa | E154K | E484Q | P681R | H1101D | Stanford-15_S02/2021 from |
| | | | | | BEI |
| B.1.617.2 | T19R, | L452R, | D614G, | D950N | hCoV-19/USA/WI-UW- |
| Delta | T95I, | T478K | P681R | | 5250/2021 |
| | G142D, | | | | |
| | E156G, | | | | |
| | F157del, | | | | |
| | R158del | | | | |
| BA.1 | A67V, | G339D, | T547K, | N764K, | hCoV-19/USA/WI-WSLH- |
| Omicron | H69del, | S371L, | D614G, | D796Y, | 221686/2021 |
| | V70del, | S373P, | H655Y, | N856K, | |
| | T95I, | S375F, | N679K, | Q954H, | |
| | G142D, | K417N, | P681H | N969K, | |
| | V143del, | N440K. | | L981F | |
| | Y144del, | G446S, | | | |
| | Y145del, | S477N, | | | |
| | N211del, | T478K, | | | |
| | L212I, | E484A, | | | |
| | ins214EPE | Q493R, | | | |
| | | G496S, | | | |
| | | Q498R, | | | |
| | | N501Y, | | | |
| | | Y505H | | | |
| BA.2 | T19I, | G339D, | D614G, | N764K, | hCoV-19/Japan/UT- |
| Omicron | delL24, | S371F, | H655Y, | D796Y, | NCD1288-2N/2022 |
| | delP25, | S373P, | N679K, | Q954H, | |
| | delP26, | S375F, | P681H | N969K | |
| | A27S, | T376A, | | | |
| | G142D, | D405N, | | | |
| | V213G | R408S, | | | |
| | | K417N, | | | |
| | | N440K, | | | |
| | | S477N, | | | |
| | | T478K, | | | |
| | | E484A, | | | |
| | | Q493R, | | | |
| | | Q498R, | | | |
| | | N501Y, | | | |
| | | Y505H | | | |
| BA.2.75 | T19I, | G339H, | D614G, | N764K, | hCoV-19/Japan/TY41- |
| Omicron | delL24, | S371F, | H655Y, | D796Y, | 716/2022 |
| | delP25, | S373P, | N679K, | Q954H, | |
| | delP26, | S375F, | P681H | N969K | |
| | A27S, | T376A, | | | |
| | G142D, | D405N, | | | |
| | K147E, | R408S, | | | |
| | W152R, | K417N, | | | |
| | F157L, | N440K, | | | |
| | I210V, | G446S, | | | |
| | V213G, | N460K, | | | |
| | G257S | S477N, | | | |
| | | T478K, | | | |
| | | E484A, | | | |
| | | Q498R, | | | |
| | | N501Y, | | | |
| | | Y505H | | | |
| BA.4 | T19I, | G339D, | D614G, | N764K, | hCoV-19/USA/MD- |
| Omicron | delL24, | S371F, | H655Y, | D796Y, | HP30386- |
| | delP25, | S373P, | N679K, | Q954H, | PIDNBNVCCQ/2022 |
| | delP26, | S375F, | P681H, | N969K | |
| | A27S, | T376A, | | | |
| | delH69, | D405N, | | | |
| | delV70, | R408S, | | | |
| | G142D, | K417N, | | | |
| | V213G | N440K, | | | |
| | | L452R, | | | |
| | | S477N, | | | |
| | | T478K, | | | |
| | | F486V, | | | |
| | | E484A, | | | |
| | | Q498R, | | | |
| | | N501Y, | | | |
| | | Y505H, | | | |
| BA.5 | T19I, | G339D, | D614G, | N764K, | SARS-CoV-2/ |
| Omicron | delL24, | S371F, | H655Y, | D796Y, | human/USA/COR-22- |
| | delP25, | S373P, | N679K, | Q954H, | 063113/2022 |
| | delP26, | S375F, | P681H, | N969K | |
| | A27S, | T376A, | | | |
| | delH69, | D405N, | | | |
| | delV70, | R408S, | | | |
| | G142D, | K417N, | | | |
| | V213G | N440K, | | | |
| | | L452R, | | | |
| | | S477N, | | | |
| | | T478K, | | | |
| | | F486V, | | | |
| | | E484A, | | | |
| | | Q498R, | | | |
| | | N501Y, | | | |
| | | Y505H, | | | |

Table S5: Pairs of S728-1157 and spike-WT-6P-Mut7 residues within predicted hydrogen bonding distances. Calculated using EpitopeAnalyzer⁶³ using a cutoff distance of 3.4 Å. Related to FIG. 9 and FIG. 15.

| | | | | | | RBD residue |
|---|---|---|---|---|---|---|
| | RBD | | | | RBD residue | conserved |
| | Residue | Ab Residue | Antibody | Distance | mutated in | across all |
| # | [Atom] | [atom] | Region | (Å) | Omicron VOC | VOC's |
| 1 | T415 [OG] | S56 [OG1] | CDRH2 | 2.78 | No | Yes |
| 2 | Y421 [OH] | S53 [O] | CDRH2 | 2.73 | No | Yes |
| 3 | Y453 [OH] | D98 [OD1] | CDRH3 | 3.5 | No | Yes |
| 4 | L455 [O] | Y33 [OH] | CDRH1 | 3.29 | No | Yes |
| 5 | R457 [O] | S53 [OG] | CDRH2 | 3.25 | No | Yes |
| 6 | Y473 [OH] | R31 [O] | CDRH1 | 2.76 | No | Yes |
| 7 | Y473 [OH] | S53 [OG] | CDRH2 | 3.26 | No | Yes |
| 8 | Q474 [O] | R31 [NH1] | CDRH1 | 3.08 | No | Yes |
| 9 | A475 [O] | L28 [N] | CDRH1 | 3.05 | No | Yes |
| 10 | A475 [O] | N32 [ND2] | CDRH1 | 2.98 | No | Yes |
| 11 | E484 [OE2] | Y99 [OH] | CDRH3 | 2.61 | Yes | No |
| 12 | N487 [ND2] | G26 [O] | FR1 | 3.01 | No | Yes |
| 13 | C488 [O] | Y99 [OH] | CDRH3 | 3.25 | No | Yes |
| 14 | Y489 [OH] | R94 [NH1] | FR3 | 2.64 | No | Yes |
| 15 | Y505 [OH] | Q31 [NE2] | CDRL1 | 2.62 | Yes | No |

Table S6. Cryo-EM data collection, refinement and model building statistics. Related to FIG. 9 and FIG. 15.

| | S728-1157 + SARS-CoV-2- | S728-1157 + SARS-CoV-2- |
|---|---|---|
| | 6P-Mut7 (global | 6P-Mut7 (focused |
| Map | refinement) | refinement) |
| EMDB | EMD-27112 | EMD-27113 |

Data collection

| | |
|---|---|
| Microscope | Thermo Fisher Titan Krios |
| Voltage (kV) | 300 |
| Detector | Gatan K2 Summit |
| Recording mode | Counting |
| Nominal magnification | 130kx |
| Movie micrograph pixelsize (Å) | 1.045 |
| Dose rate (e⁻/[(camera pixel)*s]) | 6.017 |
| Number of frames per movie micrograph | 36 |
| Frame exposure time (ms) | 250 |
| Movie micrograph exposure time (s) | 9 |
| Total dose (e⁻/Å²) | 50.0 |
| Defocus range (μm) | −0.8 to −1.5 |

EM data processing

| | | |
|---|---|---|
| Number of movie micrographs | 1,718 | 1,718 |
| Number of molecular projection images | 151,948 | 29,595 |
| in map | | |
| Symmetry | C1 | C1 |
| Map resolution (FSC 0.143; Å) | 3.3 | 3.7 |
| Map sharpening B-factor (Å²) | −85.3 | −71.1 |

Structure Building and Validation
Number of atoms in deposited model

| | | |
|---|---|---|
| SARS-CoV-2-6P-Mut7 | n/a | 20,759 |
| Fab Fv | n/a | 1,653 |
| Glycans | n/a | 182 |
| MolProbity score | n/a | 1.07 |
| Clashscore | n/a | 1.66 |
| Map correlation coefficient | n/a | 0.75 |
| EMRinger score | n/a | 2.57 |
| d FSC model (0.5; Å) | n/a | 3.8 |

RMSD from ideal

| | | |
|---|---|---|
| Bond length (Å) | n/a | 0.021 |
| Bond angles (°) | n/a | 1.81 |

Ramachandran plot

| | | |
|---|---|---|
| Favored (%) | n/a | 97.13 |
| Allowed (%) | n/a | 2.87 |
| Outliers (%) | n/a | 0.00 |
| Side chain rotamer outliers (%) | n/a | 0.08 |
| Cβ outliers (%) | n/a | 0.00 |
| PDB | n/a | 8d0z |

### E. References

The following references and the references cited throughout the specification, to the extent that they provide exemplary procedural or other details supplementary to those set forth herein, are specifically incorporated herein by reference.
1. Hou, Y. J. et al. SARS-CoV-2 D614G variant exhibits efficient replication ex vivo and transmission in vivo. Science 370, 1464-1468, doi: 10.1126/science.abe8499 (2020).
2. Garcia-Beltran, W. F. et al. Multiple SARS-CoV-2 variants escape neutralization by vaccine-induced humoral immunity. Cell 184, 2523, doi: 10.1016/j.cell.2021.04.006 (2021).
3. Wall, E. C. et al. Neutralising antibody activity against SARS-CoV-2 VOCs B.1.617.2 and B.1.351 by BNT162b2 vaccination. Lancet 397, 2331-2333, doi: 10.1016/S0140-6736 (21) 01290-3 (2021).
4. Edara, V. V. et al. Infection and vaccine-induced neutralizing-antibody responses to the SARS-CoV-2 B.1.617 variants. N Engl J Med 385, 664-666, doi: 10.1056/NEJMc2107799 (2021).
5. Zhou, D. et al. Evidence of escape of SARS-CoV-2 variant B.1.351 from natural and vaccine-induced sera. Cell 184, 2348-2361 e2346, doi: 10.1016/j.cell.2021.02.037 (2021).
6. Weisblum, Y. et al. Escape from neutralizing antibodies by SARS-CoV-2 spike protein variants. eLife 9, e61312, doi: 10.7554/eLife.61312 (2020).
7. Graham, F. Daily briefing: Omicron coronavirus variant puts scientists on alert. Nature, doi: 10.1038/d41586-021-03564-6 (2021).
8. Karim, S. S. A. & Karim, Q. A. Omicron SARS-CoV-2 variant: a new chapter in the COVID-19 pandemic. Lancet 398, 2126-2128, doi: 10.1016/S0140-6736 (21) 02758-6 (2021).
9. Carreño, J. M. et al. Activity of convalescent and vaccine serum against SARS-CoV-2 Omicron. Nature 602, 682-688, doi: 10.1038/s41586-022-04399-5 (2021).
10. VanBlargan, L. A. et al. An infectious SARS-CoV-2 B.1.1.529 Omicron virus escapes neutralization by therapeutic monoclonal antibodies. Nat Med 28, 490-495, doi: 10.1038/s41591-021-01678-y (2022).
11. Takashita, E. et al. Efficacy of antibodies and antiviral drugs against COVID-19 Omicron variant. N Engl J Med 386, 995-998, doi: 10.1056/NEJMc2119407 (2022).
12. Changrob, S. et al. Cross-neutralization of emerging SARS-CoV-2 variants of concern by antibodies targeting distinct epitopes on spike. mBio 12, e0297521, doi: 10.1128/mBio.02975-21 (2021).
13. Guthmiller, J. J. et al. SARS-CoV-2 infection severity is linked to superior humoral immunity against the spike. mBio 12, e02940-02920, doi: 10.1128/mBio.02940-20 (2021).
14. Greaney, A. J. et al. Mapping mutations to the SARS-CoV-2 RBD that escape binding by different classes of antibodies. Nat Commun 12, 4196, doi: 10.1038/s41467-021-24435-8 (2021).
15. Liu, H. & Wilson, I. A. Protective neutralizing epitopes in SARS-CoV-2. Immunol Rev, doi: 10.1111/imr.13084 (2022).
16. Jette, C. A. et al. Broad cross-reactivity across sarbecoviruses exhibited by a subset of COVID-19 donor-derived neutralizing antibodies. Cell Rep 36, 109760, doi: 10.1016/j.celrep.2021.109760 (2021).
17. Brouwer, P. J. M. et al. Potent neutralizing antibodies from COVID-19 patients define multiple targets of vulnerability. Science 369, 643-650, doi: 10.1126/science.abc5902 (2020).
18. Pinto, D. et al. Cross-neutralization of SARS-CoV-2 by a human monoclonal SARS-CoV antibody. Nature 583, 290-295, doi: 10.1038/s41586-020-2349-y (2020).
19. Robbiani, D. F. et al. Convergent antibody responses to SARS-CoV-2 in convalescent individuals. Nature 584, 437-442, doi: 10.1038/s41586-020-2456-9 (2020).
20. Yuan, M. et al. Structural basis of a shared antibody response to SARS-CoV-2. Science 369, 1119-1123, doi: 10.1126/science.abd2321 (2020).
21. Dugan, H. L. et al. Profiling B cell immunodominance after SARS-CoV-2 infection reveals antibody evolution to non-neutralizing viral targets. Immunity 54, 1290-1303, doi: 10.1016/j.immuni.2021.05.001. (2021).
22. Rogers, T. F. et al. Isolation of potent SARS-CoV-2 neutralizing antibodies and protection from disease in a small animal model. Science 369, 956-963, doi: 10.1126/science.abc7520 (2020).
23. Schmitz, A. J. et al. A vaccine-induced public antibody protects against SARS-CoV-2 and emerging variants. Immunity 54, 2159-2166.e6, doi: 10.1016/j.immuni.2021.08.013 (2021).
24. Shi, R. et al. A human neutralizing antibody targets the receptor-binding site of SARS-CoV-2. Nature 584, 120-124, doi: 10.1038/s41586-020-2381-y (2020).
25. Yuan, M., Liu, H., Wu, N. C. & Wilson, I. A. Recognition of the SARS-CoV-2 receptor binding domain by neutralizing antibodies. Biochem Biophys Res Commun 538, 192-203, doi: 10.1016/j.bbrc.2020.10.012 (2021).
26. Cao, Y. et al. Potent neutralizing antibodies against SARS-CoV-2 identified by high-throughput single-cell sequencing of convalescent patients' B cells. Cell 182, 73-84.e16, doi: 10.1016/j.cell.2020.05.025 (2020).
27. Barnes, C. O. et al. Structures of human antibodies bound to SARS-CoV-2 spike reveal epitopes and recurrent features of antibodies. Cell 182, 828-842.e6, common doi: 10.1016/j.cell.2020.06.025 (2020).
28. Corbett, K. S. et al. SARS-CoV-2 mRNA vaccine design enabled by prototype pathogen preparedness. Nature 586, 567-571, doi: 10.1038/s41586-020-2622-0 (2020).
29. Amanat, F. et al. Introduction of two prolines and removal of the polybasic cleavage site lead to higher efficacy of a recombinant spike-based SARS-CoV-2 vaccine in the mouse model. mBio 12, doi: 10.1128/mBio.02648-20 (2021).
30 Sun, W. et al. A Newcastle disease virus expressing a stabilized spike protein of SARS-CoV-2 induces protective immune responses. Nat Commun 12, 6197, doi: 10.1038/s41467-021-26499-y (2021).
31. Hsieh, C. L. et al. Structure-based design of prefusion-stabilized SARS-CoV-2 spikes. Science 369, 1501-1505, doi: 10.1126/science.abd0826 (2020).
32. Yuan, M. et al. A highly conserved cryptic epitope in the receptor binding domains of SARS-CoV-2 and SARS-CoV. Science 368, 630-633, doi: 10.1126/science.abb7269 (2020).
33. Rogers, T. F. et al. Isolation of potent SARS-CoV-2 neutralizing antibodies and protection from disease in a small animal model. Science 369, 956-963, doi: 10.1126/science.abc7520 (2020).
34. Starr, T. N., Greaney, A. J., Dingens, A. S. & Bloom, J. D. Complete map of SARS-CoV-2 RBD mutations that escape the monoclonal antibody LY-CoV555 and its cocktail with LY-CoV016. Cell Rep Med 2, 100255, doi: 10.1016/j.xcrm.2021.100255 (2021).
35 Baum, A. et al. REGN-CoV2 antibodies prevent and treat SARS-CoV-2 infection in rhesus macaques and hamsters. Science 370, 1110-1115, doi: 10.1126/science.abe2402 (2020).
36. Yuan, M. et al. Structural basis of a shared antibody response to SARS-CoV-2. Science 369, 1119-1123, doi: 10.1126/science.abd2321 (2020).
37 Wu, N. C. et al. An alternative binding mode of IGHV3-53 antibodies to the SARS-CoV-2 receptor binding domain. Cell Rep 33, 108274, doi: 10.1016/j.celrep.2020.108274 (2020).
38. Wu, Y. et al. A noncompeting pair of human neutralizing antibodies block COVID-19 virus binding to its receptor ACE2. Science 368, 1274-1278, doi: 10.1126/science.abc2241 (2020).
39 Yuan, M. et al. Structural and functional ramifications of antigenic drift in recent SARS-CoV-2 variants. Science 373, 818-823, doi: 10.1126/science.abh1139 (2021).
40. Gobeil, S. M. et al. Structural diversity of the SARS-CoV-2 Omicron spike. Mol Cell 82, 2050-2068.e6, doi: 10.1016/j.molcel.2022.03.028 (2022).
41. Yan, Q. et al. Germline IGHV3-53-encoded RBD-targeting neutralizing antibodies are commonly present in the antibody repertoires of COVID-19 patients. Emerg Microbes Infect 10, 1097-1111, doi: 10.1080/22221751.2021.1925594 (2021).
42. Barnes, C. O. et al. SARS-CoV-2 neutralizing antibody structures inform therapeutic strategies. Nature 588, 682-687, doi: 10.1038/s41586-020-2852-1 (2020).
43. Zhang, Q. et al. Potent and protective IGHV3-53/3-66 public antibodies and their shared escape mutant on the spike of SARS-CoV-2. Nat Commun 12, 4210, doi: 10.1038/s41467-021-24514-w (2021).
44. Wang, Z. et al. mRNA vaccine-elicited antibodies to SARS-CoV-2 and circulating variants. Nature 592, 616-622, doi: 10.1038/s41586-021-03324-6 (2021).
45. Starr, T. N. et al. SARS-CoV-2 RBD antibodies that maximize breadth and resistance to escape. Nature 597, 97-102, doi: 10.1038/s41586-021-03807-6 (2021).
46. Walls, A. C. et al. Structure, function, and antigenicity of the SARS-CoV-2 spike glycoprotein. Cell 181, 281-292.e6, doi: 10.1016/j.cell.2020.02.058 (2020).
47. Henderson, R. et al. Controlling the SARS-CoV-2 spike glycoprotein conformation. Nat Struct Mol Biol 27, 925-933, doi: 10.1038/s41594-020-0479-4 (2020).
48. Shrestha, L. B., Tedla, N. & Bull, R. A. Broadly-neutralizing antibodies against emerging SARS-CoV-2 variants. Front Immunol 12, 752003, doi: 10.3389/fimmu.2021.752003 (2021).
49. Greaney, A. J. et al. Antibodies elicited by mRNA-1273 vaccination bind more broadly to the receptor binding domain than do those from SARS-CoV-2 infection. Sci Transl Med 13, eabi9915, doi: 10.1126/scitranslmed.abi9915 (2021).
50 Reincke, S. M. et al. SARS-CoV-2 Beta variant infection elicits potent lineage-specific and cross-reactive antibodies. Science 375, 782-787, doi: 10.1126/science.abm5835 (2022).
51. Wrammert, J. et al. Broadly cross-reactive antibodies dominate the human B cell response against 2009 pandemic H1N1 influenza virus infection. J Exp Med 208, 181-193, doi: 10.1084/jem.20101352 (2011).
52. Guthmiller, J. J. et al. First exposure to the pandemic H1N1 virus induced broadly neutralizing antibodies targeting hemagglutinin head epitopes. Sci Transl Med 13, eabg4535, doi: 10.1126/scitranslmed.abg4535 (2021).
53 Bajic, G. et al. Influenza antigen engineering focuses immune responses to a subdominant but broadly protective viral epitope. Cell Host Microbe 25, 827-835.e6, doi: 10.1016/j.chom.2019.04.003 (2019).
54. Nachbagauer, R. et al. A chimeric hemagglutinin-based universal influenza virus vaccine approach induces broad and long-lasting immunity in a randomized, placebo-controlled phase I trial. Nat Med 27, 106-114, doi: 10.1038/s41591-020-1118-7 (2021).
55. Angeletti, D. et al. Outflanking immunodominance to target subdominant broadly neutralizing epitopes. Proc Natl Acad Sci USA 116, 13474-13479, doi: 10.1073/pnas. 1816300116 (2019).
56. Guthmiller, J. J., Dugan, H. L., Neu, K. E., Lan, L. Y. & Wilson, P. C. An efficient method to generate monoclonal antibodies from human B cells. Methods Mol Biol 1904, 109-145, doi: 10.1007/978-1-4939-8958-4_5 (2019).
57. Amanat, F. et al. A serological assay to detect SARS-CoV-2 seroconversion in humans. Nat Med 26, 1033-1036, doi: 10.1038/s41591-020-0913-5 (2020).
58. Stadlbauer, D. et al. SARS-CoV-2 seroconversion in humans: A detailed protocol for a serological assay, antigen production, and test setup. Curr Protoc Microbiol 57, e100, doi: 10.1002/cpmc.100 (2020).
59. Torres, J. L. et al. Structural insights of a highly potent pan-neutralizing SARS-CoV-2 human monoclonal antibody. Proc Natl Acad Sci USA 119, e2120976119, doi: 10.1073/pnas.2120976119 (2022).
60. Suloway, C. et al. Automated molecular microscopy: the new Leginon system. J Struct Biol 151, 41-60, doi: 10.1016/j.jsb.2005.03.010 (2005).
61. Lander, G. C. et al. Appion: an integrated, database-driven pipeline to facilitate EM image processing. J Struct Biol 166, 95-102, doi: 10.1016/j.jsb.2009.01.002 (2009).
62. Voss, N. R., Yoshioka, C. K., Radermacher, M., Potter, C. S. & Carragher, B. DoG Picker and TiltPicker: software tools to facilitate particle selection in single particle electron microscopy. J Struct Biol 166, 205-213, doi: 10.1016/j.jsb.2009.01.004 (2009).
63 Pettersen, E. F. et al. UCSF Chimera-A visualization system for exploratory research and analysis. J Comput Chem 25, 1605-1612, doi: 10.1002/jcc.20084 (2004).
64. Punjani, A., Zhang, H. & Fleet, D. J. Non-uniform refinement: adaptive regularization improves single-particle cryo-EM reconstruction. Nat Methods 17, 1214-1221, doi: 10.1038/s41592-020-00990-8 (2020).
65. Zhang, K. Gctf: Real-time CTF determination and correction. J Struct Biol 193, 1-12, doi: 10.1016/j.jsb.2015.11.003 (2016).
66. Zivanov, J. et al. New tools for automated high-resolution cryo-EM structure determination in RELION-3. eLife 7, e42166, doi: 10.7554/eLife.42166 (2018).
67. Casanal, A., Lohkamp, B. & Emsley, P. Current developments in Coot for macromolecular model building of electron cryo-microscopy and crystallographic data. Protein Sci 29, 1069-1078, doi: 10.1002/pro.3791 (2020).
68. Frenz, B. et al. Automatically fixing errors in glycoprotein structures with Rosetta. Structure 27, 134-139.e3, doi: 10.1016/j.str.2018.09.006 (2019).
69. Klaholz, B. P. Deriving and refining atomic models in crystallography and cryo-EM: the latest Phenix tools to facilitate structure analysis. Acta Crystallogr D Struct Biol 75, 878-881, doi: 10.1107/S2059798319013391 (2019).
70. Pettersen, E. F. et al. UCSF ChimeraX: Structure visualization for researchers, educators, and developers. Protein Sci 30, 70-82, doi: 10.1002/pro.3943 (2021).
71. Otwinowski, Z. & Minor, W. Processing of X-ray diffraction data collected in oscillation mode. Methods Enzymol 276, 307-326 (1997).
72. McCoy, A. J. et al. Phaser crystallographic software. J Appl Crystallogr 40, 658-674, doi: 10.1107/S0021889807021206 (2007).
73 Qiang, M. et al. Neutralizing antibodies to SARS-CoV-2 selected from a human antibody library constructed decades ago. Adv Sci (Weinh) 9, e2102181, doi: 10.1002/advs.202102181 (2022).
74. Emsley, P. & Cowtan, K. Coot: model-building tools for molecular graphics. Acta Crystallogr D Biol Crystallogr 60, 2126-2132, doi: 10.1107/S0907444904019158 (2004).
75. Adams, P. D. et al. PHENIX: a comprehensive Python-based system for macromolecular structure solution. Acta Crystallogr D Biol Crystallogr 66, 213-221, doi: 10.1107/S0907444909052925 (2010).
76. Montiel-Garcia, D., Rojas-Labra, O., Santoyo-Rivera, N. & Reddy, V. S. Epitope-Analyzer: A structure-based webtool to analyze broadly neutralizing epitopes. J Struct Biol 214, 107839, doi: 10.1016/j.jsb.2022.107839 (2022).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. An antibody or antigen binding fragment comprising a heavy chain variable region and a light chain variable region:
(i) wherein the heavy chain variable region comprises a HCDR1, HCDR2, and HCDR3 having at least 80% sequence identity to the HCDR1, HCR2, HCR3 of SEQ ID NOs: 1565, 1566, and 1567 and wherein the light chain variable region comprises a LCDR1, LCDR2, and LCDR3 having at least 80% sequence identity to the LCDR1, LCDR2, and LCDR3 of SEQ ID NOs: 1572, 1573, and 1574;
(ii) wherein the heavy chain variable region comprises a HCDR1, HCDR2, and HCDR3 having at least 80% sequence identity to the HCDR1, HCR2, HCR3 of SEQ ID NOs: 1457, 1458, and 1459 and wherein the light chain variable region comprises a LCDR1, LCDR2, and LCDR3 having at least 80% sequence identity to the LCDR1, LCDR2, and LCDR3 of SEQ ID NOs: 1464, 1465, and 1466; or
(iii) wherein the heavy chain variable region comprises a HCDR1, HCDR2, and HCDR3 having at least 80% sequence identity to the HCDR1, HCR2, HCR3 of SEQ ID NOs: 1492, 243, and 1493 and wherein the light chain variable region comprises a LCDR1, LCDR2, and LCDR3 having at least 80% sequence identity to the LCDR1, LCDR2, and LCDR3 of SEQ ID NOs: 1497, 1498, and 1499.

2. The antibody or antigen binding fragment of claim 1:
(i) wherein the heavy chain variable region comprises a HCDR1, HCDR2, and HCDR3 having the amino acid sequence of SEQ ID NOs: 1565, 1566, and 1567 and wherein the light chain variable region comprises a LCDR1, LCDR2, and LCDR3 having the amino acid sequence of SEQ ID NOs: 1572, 1573, and 1574;
(ii) wherein the heavy chain variable region comprises a HCDR1, HCDR2, and HCDR3 having the amino acid sequence of SEQ ID NOs: 1457, 1458, and 1459 and wherein the light chain variable region comprises a LCDR1, LCDR2, and LCDR3 having the amino acid sequence of SEQ ID NOs: 1464, 1465, and 1466; or
(iii) wherein the heavy chain variable region comprises a HCDR1, HCDR2, and HCDR3 having the amino acid sequence of SEQ ID NOs: 1492, 243, and 1493 and wherein the light chain variable region comprises a LCDR1, LCDR2, and LCDR3 having the amino acid sequence of SEQ ID NOs: 1497, 1498, and 1499.

3. The antibody or antigen binding fragment of claim 1 or 2:
(i) wherein the heavy chain comprises an amino acid sequence with at least 80% sequence identity to SEQ ID NO: 1563 or 1564 and/or the light chain comprises an amino acid sequence with at least 80% sequence identity to SEQ ID NO:1570 or 1571;
(ii) wherein the heavy chain comprises an amino acid sequence with at least 80% sequence identity to SEQ ID NO: 1455 or 1456 and/or the light chain comprises an amino acid sequence with at least 80% sequence identity to SEQ ID NO:1462 or 1463; or
(iii) wherein the heavy chain comprises an amino acid sequence with at least 80% sequence identity to SEQ ID NO: 1490 or 1491 and/or the light chain comprises an amino acid sequence with at least 80% sequence identity to SEQ ID NO: 1495 or 1496.

4. The antibody or antigen binding fragment of claim 3:
(i) wherein the heavy chain comprises the amino acid sequence of SEQ ID NO: 1563 or 1564 and/or the light chain comprises the amino acid sequence of SEQ ID NO: 1570 or 1571;
(ii) wherein the heavy chain comprises the amino acid sequence of SEQ ID NO: 1455 or 1456 and/or the light chain comprises the amino acid sequence of SEQ ID NO: 1462 or 1463; or
(iii) wherein the heavy chain comprises the amino acid sequence of SEQ ID NO: 1490 or 1491 and/or the light chain comprises the amino acid sequence of SEQ ID NO: 1495 or 1496.

5. The antibody or antigen binding fragment of any one of claims 1-4, wherein the antibody or antigen binding fragment comprises:
(i) a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence with at least 80% sequence identity to SEQ ID NOs: 1568, 130, 1569, and 60, respectively, and the LFR1, LFR2, LFR3, and LFR4 comprises an amino acid sequence with at least 80% sequence identity to SEQ ID NOs: 1575, 950, 1576, and 69;
(ii) a heavy chain framework region HFR1, HFR2, HFR3, and HFR4 and light chain framework region LFR1, LFR2, LFR3, and LFR4, and wherein the HFR1, HFR2, HFR3, and HFR4 comprises an amino acid sequence with at least 80% sequence identity to SEQ ID NOs: 1460, 1461, 146, and 60, respectively, and the LFR1, LFR2, LFR3, and LFR4 comprises an amino acid sequence with at least 80% sequence identity to SEQ ID NOs: 1467, 1468, 1469, and 53; or
(iii) a heavy chain framework region HFR1, HFR2, HFR3, and HFR4 and light chain framework region LFR1, LFR2, LFR3, and LFR4, and wherein the HFR1, HFR2, HFR3, and HFR4 comprises an amino acid sequence with at least 80% sequence identity to SEQ ID NOs: 245, 7, 1494, and 44, respectively, and the LFR1, LFR2, LFR3, and LFR4 comprises an amino acid sequence with at least 80% sequence identity to SEQ ID NOs: 1500, 1501, 1502, and 18.

6. The antibody or antigen binding fragment of any one of claims 1-5:
(i) wherein the HFR1, HFR2, HFR3, and HFR4 comprises the amino acid sequence of SEQ ID NOs: 1568, 130, 1569, and 60, and the LFR1, LFR2, LFR3, and LFR4 comprises the amino acid sequence of SEQ ID NOs: 1575, 950, 1576, and 69;
(ii) wherein the HFR1, HFR2, HFR3, and HFR4 comprises the amino acid sequence of SEQ ID NOs: 1460, 1461, 146, and 60, and the LFR1, LFR2, LFR3, and LFR4 comprises the amino acid sequence of SEQ ID NOs: 1467, 1468, 1469, and 53; or
(iii) wherein the HFR1, HFR2, HFR3, and HFR4 comprises the amino acid sequence of SEQ ID NOs: 245, 7, 1494, and 44, and the LFR1, LFR2, LFR3, and LFR4 comprises the amino acid sequence of SEQ ID NOs: 1500, 1501, 1502, and 18.

7. An antibody or antigen binding fragment comprising a heavy chain variable region and a light chain variable region, wherein the heavy chain variable region comprises a HCDR1, HCDR2, and HCDR3 having at least 80% sequence identity to the HCDR1, HCR2, HCR3 from a heavy chain variable region of a antibody clone of Table 1 and wherein the light chain variable region comprises a LCDR1, LCDR2, and LCDR3 having at least 80% sequence identity to the LCDR1, LCDR2, and LCDR3 from the light chain variable region of the same antibody clone of Table 1.

8. The antibody or antigen binding fragment of claim 7, wherein the heavy chain variable region comprises a HCDR1, HCDR2, and HCDR3 having the amino acid sequence of an of a HCDR1, HCDR2, and HCDR3 of a clone of Table 1 and wherein the light chain variable region comprises a LCDR1, LCDR2, and LCDR3 comprising the amino acid sequence of the LCDR1, LCDR2, and LCDR3 from the light chain variable region of the same clone of Table 1.

9. The antibody or antigen binding fragment of claim 7 or 8, wherein the HCDR1, HCDR2, HCDR2, LCDR1, LCDR2, and LCDR3 each comprise an amino acid sequence that has at least 80% sequence identity to an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone.

10. The antibody or antigen binding fragment of claim 7 or 8, wherein the HCDR1, HCDR2, HCDR2, LCDR1, LCDR2, and LCDR3 each comprise the amino acid sequence of an HCDR1, HCDR2, HCDR2, LCDR1, LCDR2, and LCDR3 of Table 1, wherein the HCDR1, HCDR2, HCDR2, LCDR1, LCDR2, and LCDR3 are from the same antibody clone.

11. The antibody or antigen binding fragment of any one of claims 7-10, wherein the heavy chain variable region comprises an amino acid sequence with at least 80% sequence identity to a heavy chain variable region of an antibody clone of Table 1 and/or the light chain variable region comprises an amino acid sequence with at least 80% sequence identity to the light chain variable region of the same antibody clone of Table 1.

12. The antibody or antigen binding fragment of claim 11, wherein the heavy chain variable region comprises the amino acid sequence of a heavy chain variable region of an antibody clone of Table 1 and/or the light chain variable region comprises the amino acid sequence of the same antibody clone of Table 1.

13. The antibody or antigen binding fragment of any one of claims 7-12, wherein the antibody or antigen binding fragment comprises a heavy chain framework region (HFR) 1, HFR2, HFR3, and HFR4 and light chain framework region (LFR) 1, LFR2, LFR3, and LFR4, and wherein the HFR1, HFR2, HFR3, and HFR4 comprises an amino acid sequence with at least 80% sequence identity to an HFR1, HFR2, HFR3, and HFR4, respectively, of an antibody clone of Table 1, and the LFR1, LFR2, LFR3, and LFR4 comprises an amino acid sequence with at least 80% sequence identity to the LFR1, LFR2, LFR3, and LFR4, respectively, of the same antibody clone of Table 1.

14. The antibody or antigen binding fragment of any one of claims 7-12, wherein the HFR1, HFR2, HFR3, and HFR4 comprises the amino acid sequence of an HFR1, HFR2, HFR3, and HFR4, respectively, of an antibody clone of Table 1, and the LFR1, LFR2, LFR3, and LFR4 comprises the amino acid sequence of the LFR1, LFR2, LFR3, and LFR4, respectively, of the same antibody clone of Table 1.

15. The antibody or antigen binding fragment of any one of claims 7-14, wherein the antibody comprises a heavy chain and a light chain and wherein the heavy chain comprises an amino acid sequence with at least 70% sequence identity to a heavy chain of an antibody clone of Table 1 and the light chain comprises an amino acid sequence with at least 70% sequence identity to the light chain of the same antibody clone of Table 1.

16. The antibody or antigen binding fragment of claim 15, wherein the antibody comprises a heavy chain and a light chain and wherein the heavy chain comprises the amino acid sequence of an antibody clone of Table 1 and the light chain comprises the amino acid sequence of the same antibody clone of Table 1.

17. The antibody of any one of claims 1-16, wherein the antibody is human, chimeric, or humanized.

18. The antibody or antigen-binding fragment of any one of claims 1-17, wherein the antibody, or antigen binding fragment binds a SARS-CoV-2 protein with a K_{D} of about 10⁻⁶ nM to about 10⁻¹² pM.

19. The antibody or antigen binding fragment of any one of claims 1-18, wherein the antibody is a neutralizing antibody.

20. The antibody or antigen binding fragment of any one of claims 1-19, wherein the antibody is a human antibody, humanized antibody, recombinant antibody, chimeric antibody, an antibody derivative, a veneered antibody, a diabody, a monoclonal antibody, a single domain antibody, or a single chain antibody.

21. The antigen binding fragment of any one of claims 1-19, wherein the antigen binding fragment is a single chain variable fragment (scFv), F(ab′)2, Fab′, Fab, Fv, or rIgG.

22. A polypeptide comprising the antigen binding fragment of any one of claims 1-21.

23. The polypeptide of claim 22, wherein the polypeptide comprises at least two antigen binding fragments, wherein each antigen binding fragment is independently selected from an antigen binding fragment of any one of claims 1-21.

24. The polypeptide of claim 22 or 23, wherein the polypeptide is multivalent.

25. The polypeptide of any one of claims 22-24, wherein the polypeptide is bispecific.

26. A composition comprising the antibody or antigen binding fragment of any one of claims 1-25.

27. The composition of claim 26, wherein the composition comprises a pharmaceutical excipient.

28. The composition of claim 26 or 27, wherein the composition further comprises an adjuvant.

29. The composition of any one of claims 26-28, wherein the composition is formulated for parenteral, intravenous, subcutaneous, intramuscular, or intranasal administration.

30. The composition of any one of claims 26-29, wherein the composition comprises at least two antibodies or antigen binding fragments.

31. One or more nucleic acids encoding the antibody or antigen binding fragment of any one of claims 1-21 or the polypeptide of claim 25.

32. A nucleic acid encoding an antibody heavy chain, wherein the nucleic acid has at least 70% sequence identity to one of the nucleic acid sequences of a heavy chain of Table 2.

33. A nucleic acid encoding an antibody light chain, wherein the nucleic acid has at least 70% sequence identity to one of the nucleic acid sequences of a light chain of Table 2.

34. A vector comprising the nucleic acid(s) of any one of claims 31-33.

35. A host cell comprising the nucleic acid of any one of claims 31-33 or the vector of claim 34.

36. The host cell of claim 35, wherein the host cell is a human cell, B cell, T cell, Chinese hamster ovary, NS0 murine myeloma cell, or PER.C6 cell.

37. A method of a making a cell comprising transferring the nucleic acid(s) of any one of claims 31-33 or the vector of claim 34 into a cell.

38. The method of claim 37, wherein the method further comprises culturing the cell under conditions that allow for expression of a polypeptide from the nucleic acid.

39. The method of claim 38, wherein the method further comprising isolating the expressed polypeptide.

40. The method of any one of claims 37-39, wherein the cell is a human cell, B cell, T cell, Chinese hamster ovary, NS0 murine myeloma cell, or PER.C6 cell.

41. A method for producing a polypeptide comprising culturing cells comprising the nucleic acid(s) of any one of claims 31-33 or the vector of claim 34 and isolating polypeptides expressed from the nucleic acid.

42. The method of claim 41, wherein the cell is a human cell, B cell, T cell, Chinese hamster ovary, NS0 murine myeloma cell, or PER.C6 cell.

43. A method for treating or preventing a coronavirus infection in a subject, the method comprising administering to the subject, the antibody or antigen binding fragment of any one of claims 1-21, the polypeptide of claim 25, or the host cell of claim 35.

44. The method of claim 43, wherein the subject is a human subject.

45. The method of claim 43 or 44, wherein the coronavirus infection is SARS-CoV-2.

46. The method of claim 43 or 44, wherein the subject has one or more symptoms of a coronavirus infection.

47. The method of claim 43 or 44, wherein the subject does not have any symptoms of a coronavirus infection.

48. The method of any one of claims 43-47, wherein the subject has been diagnosed with a coronavirus infection.

49. The method of any one of claims 43-47, wherein the subject has not been diagnosed with a coronavirus infection.

50. The method of any one of claims 43-49, wherein the subject has been previously vaccinated for coronavirus.

51. The method of any one of claims 43-49, wherein the subject has not been previously vaccinated for coronavirus.

52. The method of any one of claims 43-51, wherein the antibody, antigen binding fragment, polypeptide, or cell is administered by parenteral, intravenous, subcutaneous, intramuscular, or intranasal administration.

53. The method of any one of claims 43-49, wherein the subject has been previously treated for a coronavirus infection.

54. The method of any one of claims 43-53, wherein the subject is administered an additional therapeutic.

55. The method of claim 54, wherein the additional therapeutic comprises a steroid or an anti-viral therapeutic.

56. The method of claim 55, wherein the additional therapeutic comprises dexamethasone or remdesivir.

57. A method for evaluating a sample from a subject, the method comprising contacting a biological sample from the subject, or extract thereof, with at least one antibody, antigen binding fragment, or polypeptide of any one of claims 1-25.

58. The method of claim 57, wherein the at least one antibody, antigen binding fragment, or polypeptide is operatively linked to a detectable label.

59. The method of claim 57 or 58, wherein the method further comprises incubating the antibody, antigen binding fragment, or polypeptide under conditions that allow for the binding of the antibody, antigen binding fragment, or polypeptide to antigens in the biological sample or extract thereof.

60. The method of any one of claims 57-59, wherein the method further comprises detecting the binding of an antigen to the antibody, antigen binding fragment, or polypeptide.

61. The method of any one of claims 57-60, wherein the method further comprises contacting the biological sample with at least one capture antibody, antigen, or polypeptide.

62. The method of claim 61, wherein the at least one capture antibody, antigen binding fragment, or polypeptide comprises at least one antibody of claims 7-25.

63. The method of claim 61 or 62, wherein the capture antibody is linked to a solid support.

64. The method of any one of claims 57-63, wherein the biological sample comprises a blood sample, urine sample, fecal sample, or nasopharyngeal sample.

65. A method for diagnosing a SARS-CoV-2 infection in a subject, the method comprising contacting a biological sample from the subject, or extract thereof, with at least one antibody, antigen binding fragment, or polypeptide of any one of claims 7-25.

66. The method of claim 65, wherein the at least one antibody, antigen binding fragment, or polypeptide is operatively linked to a detectable label.

67. The method of claim 65 or 66, wherein the method further comprises incubating the antibody, antigen binding fragment, or polypeptide under conditions that allow for the binding of the antibody, antigen binding fragment, or polypeptide to antigens in the biological sample or extract thereof.

68. The method of any one of claims 65-67, wherein the method further comprises detecting the binding of an antigen to the antibody, antigen binding fragment, or polypeptide.

69. The method of any one of claims 65-68, wherein the method further comprises contacting the biological sample with at least one capture antibody, antigen, or polypeptide.

70. The method of claim 69, wherein the at least one capture antibody, antigen, or polypeptide comprises at least one antibody, antigen, or polypeptide of claims 7-25.

71. The method of claim 69 or 70, wherein the capture antibody is linked to a solid support.

72. The method of any one of claims 65-71, wherein the biological sample comprises a blood sample, urine sample, fecal sample, or nasopharyngeal sample.
